(19) United States
(12) Patent Application Publication
(10) Pub. No.: US 2024/0398828 A1
(43) Pub. Date: Dec. 5, 2024

(54) HETERO-ATOM CONTAINING COMPOUNDS AND USES THEREOF

(71) Applicant: Kumquat Biosciences Inc., San Diego, CA (US)

(72) Inventors: Liansheng Li, San Diego, CA (US); Xiuwen Zhu, San Diego, CA (US); Pingda Ren, San Diego, CA (US); Jonathan Deane, San Diego, CA (US); Lomon So, Poway, CA (US); Yi Liu, San Diego, CA (US)

(21) Appl. No.: 18/669,349

(22) Filed: May 20, 2024

Related U.S. Application Data
(63) Continuation of application No. PCT/US2022/050777, filed on Nov. 22, 2022.
(60) Provisional application No. 63/282,614, filed on Nov. 23, 2021.
(60) Provisional application No. 63/406,215, filed on Sep. 13, 2022.

(51) Int. Cl. A61K 31/553; A61K 31/433; A61K 31/437; A61K 31/439; A61K 31/4439; A61K 31/4725; A61K 31/496; A61K 31/506; A61K 45/06; A61P 37/04; C07D 285/06; C07D 417/10; C07D 417/14; C07D 471/08; C07D 487/10; C07D 491/107

(52) U.S. Cl. CPC A61K 31/553; A61K 31/433; A61K 31/437; A61K 31/439; A61K 31/4439; A61K 31/4725; A61K 31/496; A61K 31/506; A61K 45/06; A61P 37/04; C07D 285/06; C07D 417/10; C07D 417/14; C07D 471/08; C07D 487/10; C07D 491/107

(57) ABSTRACT

The present disclosure provides compounds and pharmaceutical compositions comprising the same. The compounds, pharmaceutical compositions thereof, and methods of using the same have a range of utilities as therapeutics, diagnostics, and research tools. The subject compositions and methods are particularly useful for potentiating immune response and/or applicable for treating cancer and other diseases.

### CROSS-REFERENCE

This application is a continuation of International Application No. PCT/US2022/050777, filed Nov. 22, 2022, which claims the benefit of U.S. Provisional Patent Application Nos. 63/282,614 filed on Nov. 23, 2021 and 63/406,215 filed on Sep. 13, 2022, each of which is incorporated by reference in its entirety.

### BACKGROUND

PTPN2 encodes a protein tyrosine phosphatase that has been implicated in a number of intracellular signaling pathways of immune cells. PTPN2 can negatively regulate αβ TCR T cell receptor (TCR) signaling by dephosphorylating and inactivating, e.g., the Src family kinase including LCK. In addition, PTPN2 can antagonize growth factor or cytokine-mediated signaling required for T cell function, homeostasis, and/or differentiation by dephosphorylating and inactivating JAK family kinases, e.g., JAK-1 and JAK-3, and/or target substrates of the JAK family kinases, e.g., STAT-1, STAT-3, and STAT-5.

Based on genome-wide association studies, PTPN2 single nucleotide polymorphisms (SNPs) have been linked with the development of several human autoimmune diseases including, but not limited to, type 1 diabetes, rheumatoid arthritis, Crohn's disease, and celiac disease. For example, a PTPN2 variant, rs1893217(C), has been associated with about a 40% decrease in PTPN2 mRNA expression in CD4+ T cells, as well as the development of type 1 diabetes. In addition, PTPN2 mRNA expression levels in lung cancer tissues have been shown to be higher than those in normal lung tissues or adjacent normal tissues, such overexpression of PTPN2 promoting proliferation of lung cancer cells. Furthermore, two PTPN2 SNPs, rs2847297 and rs2847282, have been associated with a decrease in both PTPN2 mRNA expression and lung cancer risk, especially squamous cell lung carcinoma risk.

Cancer is the second leading cause of human death. There were close to 10 million deaths from cancer worldwide in 2018 and 17 million new cases were diagnosed. In the United States alone, cancer causes the death of over a half-million people annually, with some 1.7 million new cases diagnosed per year (excluding basal cell and squamous cell skin cancers). Lung, liver, stomach, and bowel cancers account for more than four in ten of all cancer deaths worldwide.

Adoptive transfer of gene modified lymphoid cells, particularly T cells (i.e., ACT), is an emerging treatment for cancer. While efficacy has been demonstrated in a range of hematological cancers including ALL, CLL, DLBCL, FL, and multiple myeloma, its efficacy in treating solid tumors is still yet to be established. Current immune cell therapy (e.g., CAR-T therapy) suffers from a number of profound deficiencies. T cell manufacturing and clonal expansion are highly inefficient and costly. When introduced in to a patient, T cell's anti-tumor activity and numbers can be reduced in the immunosuppressive microenvironment often found in a tumor. In addition, CAR-T therapy has been limited by life threatening toxicities in over 30% of patients. Toxicities primarily manifest as cytokine release syndrome (CRS) characterized by an early phase with fever, hypotension and elevations of various cytokines, and a later phase associated with life-ending neurologic events.

### SUMMARY

In view of the foregoing, there exists a considerable need for alternative compositions and methods to treat cancer, and/or carry out immunotherapy. The compositions and methods of the present disclosure address this need and provide additional advantages as well. The ability of PTPN2 to act as a negative regulator of immunoreceptor-related pathways (e.g., TCR signaling) and promote cancer cell proliferation can be exploited for cancer and tumor treatment. The various aspects of the disclosure provide compositions and methods for inducing activity of lymphoid cells.

In an aspect is provided a compound of Formula (I), or a pharmaceutically acceptable salt or solvate thereof:

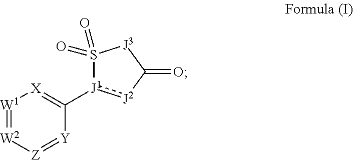

either: W¹ is C(R¹), and W² is C(R^{2a}) or N; or W¹ is C(R^{1a}) or N, and W² is C(R²);
R¹ is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are substituted with one or more substituents independently selected from R⁶ and R^{6′}, wherein ring heteroatoms of the 5 to 12 membered heterocycloalkyl and 5 to 12 membered heteroaryl are independently selected from N, O, and S;
R^{1a} is selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20a};
R² is selected from C₅₋₁₂cycloalkyl, 5 to 12 membered heterocycloalkyl, C₆₋₁₂aryl, and 5 to 12 membered heteroaryl, wherein C₁₋₁₂cycloalkyl, 5 to 12 membered heterocycloalkyl, C₆₋₁₂aryl, and 5 to 12 membered heteroaryl are substituted with one or more substituents independently selected from R⁷ and R^{7′}, wherein ring heteroatoms of the 5 to 12 membered heterocycloalkyl and 5 to 12 membered heteroaryl are independently selected from N, O, and S;
R^{2a} are independently selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20b};
X is N or C(R³);
Y is N or C(R⁴).
Z is N or C(R⁵);
R³ is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R⁴ is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_g S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20e};
each R⁶ and R⁷ are independently selected from halogen, oxo, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁶;
each R^{6′} and R^{7′} are independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀, aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶;
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉ heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉ heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20l};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted with one, two, or three R^{20m};
each R¹⁶ is independently selected from —C₁₋₆alkylene-OP(O)(OR^{16a})(OR¹⁶ⁱ*) and —P(O)(OR^{16a})(OR^{16b}); wherein the C₁₋₆alkylene is optionally substituted with one, two, or three R²⁰ⁿ; or —C₁₋₆alkylene-OC(O)—R^{16c} wherein C₁₋₆alkylene is optionally substituted with one, two, or three R²⁰ⁿ;
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20o};
R^{16c} is halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —CH₂—C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three of R^{20o};
each R^{20a}, R^{20b}, R^{20c}, R^{20d}, R^{20e}, R^{20f}, R^{20h}, R²⁰ⁱ, R^{20j}, R^{20k}, R^{20l}, R^{20m}, R²⁰ⁿ, and R^{20o} are each independently selected from oxo, —CN, halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl optionally substituted with one or more R²⁶, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀ aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each Ru is independently selected from H and C₁₋₆alkyl;
each R²⁵ is selected from C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl;
each R²⁶ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁷;
each R²⁷ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20f};
[symbol] indicates a single or double bond such that all valences are satisfied.

In embodiments of the subject compound (i.e., compound described herein), or a pharmaceutically acceptable salt or solvate thereof, X is C(R³). In embodiments of the subject compound (i.e., compound described herein), or a pharmaceutically acceptable salt or solvate thereof, Y is C(R⁴). In embodiments of the subject compound (i.e., compound described herein), or a pharmaceutically acceptable salt or solvate thereof, Z is C(R⁵).

In embodiments of the subject compound (i.e., compound described herein), or a pharmaceutically acceptable salt or solvate thereof, X is N. In embodiments of the subject compound (i.e., compound described herein), or a pharmaceutically acceptable salt or solvate thereof, Y is N. In embodiments of the subject compound (i.e., compound described herein), or a pharmaceutically acceptable salt or solvate thereof, wherein Z is N.

In embodiments of the subject compound (i.e., compound described herein), or a pharmaceutically acceptable salt or solvate thereof, W¹ is C(R¹). In embodiments of the subject compound (i.e., compound described herein), or a pharmaceutically acceptable salt or solvate thereof, W¹ is C(R^{1a}). In embodiments of the subject compound (i.e., compound described herein), or a pharmaceutically acceptable salt or solvate thereof, W² is C(R^{2a}). In embodiments of the subject compound (i.e., compound described herein), or a pharmaceutically acceptable salt or solvate thereof, W² is C(R²). In embodiments of the subject compound (i.e., compound described herein), or a pharmaceutically acceptable salt or solvate thereof, W¹ is N. In embodiments of the subject compound (i.e., compound described herein), or a pharmaceutically acceptable salt or solvate thereof, W² is N.

In embodiments of the subject compound (i.e., compound described herein), or a pharmaceutically acceptable salt or solvate thereof, J² is CH₂. In embodiments of the subject compound (i.e., compound described herein), or a pharmaceutically acceptable salt or solvate thereof, J² is C(H). In embodiments of the subject compound (i.e., compound described herein), or a pharmaceutically acceptable salt or solvate thereof, J³ is N(H). In embodiments of the subject compound (i.e., compound described herein), or a pharmaceutically acceptable salt or solvate thereof, J³ is C(R¹⁰)(R^{10a}). In embodiments of the subject compound (i.e., compound described herein), or a pharmaceutically acceptable salt or solvate thereof, J³ is CH(R¹⁰). In embodiments of the subject compound (i.e., compound described herein), or a pharmaceutically acceptable salt or solvate thereof, R¹⁰ is hydrogen.

In embodiments of the subject compound (i.e., compound described herein), or a pharmaceutically acceptable salt or solvate thereof, the compound has the structure of Formula (Ia):

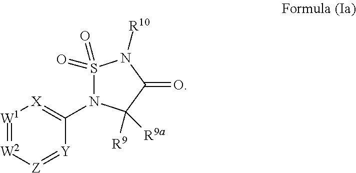

In embodiments of the subject compound (i.e., compound described herein), or a pharmaceutically acceptable salt or solvate thereof, the compound has the structure of Formula (Ib):

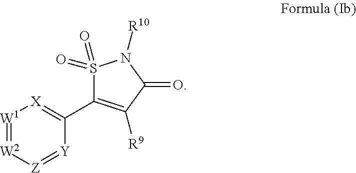

In embodiments of the subject compound (i.e., compound described herein), or a pharmaceutically acceptable salt or solvate thereof, the compound has the structure of Formula (Ic):

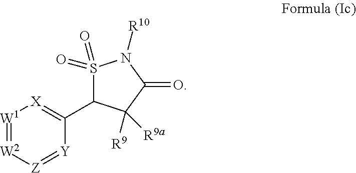

In embodiments of the subject compound (i.e., compound described herein), or a pharmaceutically acceptable salt or solvate thereof, the compound has the formula:

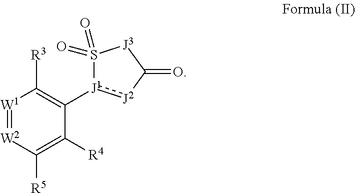

In embodiments of the subject compound (i.e., compound described herein), or a pharmaceutically acceptable salt or solvate thereof, the compound has the structure of Formula (IIa):

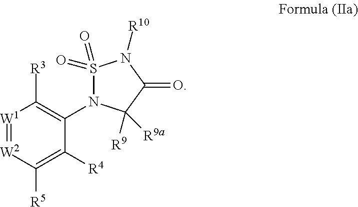

In embodiments of the subject compound (i.e., compound described herein), or a pharmaceutically acceptable salt or solvate thereof, the compound has the structure of Formula (IIb):

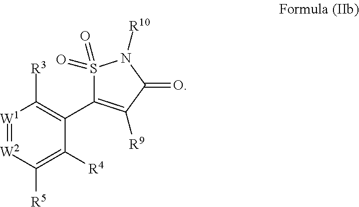

In embodiments of the subject compound (i.e., compound described herein), or a pharmaceutically acceptable salt or solvate thereof, the compound has the structure of Formula (IIc):

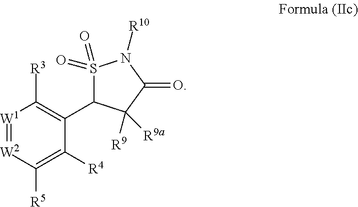

In embodiments of the subject compound (i.e., compound described herein), or a pharmaceutically acceptable salt or solvate thereof, R⁴ is selected from halogen, —OR¹², and C₁₋₆alkyl optionally substituted with one, two, or three R^{20d}. In embodiments of the subject compound (i.e., compound described herein), or a pharmaceutically acceptable salt or solvate thereof, R⁴ is —OH.

In embodiments of the subject compound (i.e., compound described herein), or a pharmaceutically acceptable salt or solvate thereof, R⁵ is selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20e}. In embodiments of the subject compound (i.e., compound described herein), or a pharmaceutically acceptable salt or solvate thereof, R⁵ is selected from hydrogen, halogen, and C₁₋₆alkyl optionally substituted with one, two, or three R^{20e}. In embodiments of the subject compound (i.e., compound described herein), or a pharmaceutically acceptable salt or solvate thereof, R⁵ is hydrogen.

In embodiments of the subject compound (i.e., compound described herein), or a pharmaceutically acceptable salt or solvate thereof, R³ is selected from halogen and C₁₋₆alkyl optionally substituted with one, two, or three R^{20c}. In embodiments of the subject compound (i.e., compound described herein), or a pharmaceutically acceptable salt or solvate thereof, R³ is halogen. In embodiments of the subject compound (i.e., compound described herein), or a pharmaceutically acceptable salt or solvate thereof, R³ is F. In embodiments of the subject compound (i.e., compound described herein), or a pharmaceutically acceptable salt or solvate thereof, R³ is Cl.

In embodiments of the subject compound (i.e., compound described herein), or a pharmaceutically acceptable salt or solvate thereof, R¹ is selected from C₅₋₁₂cycloalkyl, 5 to 12 membered heterocycloalkyl, C₇₋₁₂aryl, and 5 to 12 membered heteroaryl, wherein C₅₋₁₂cycloalkyl, 5 to 12 membered heterocycloalkyl, C₇₋₁₂aryl, and 5 to 12 membered heteroaryl are substituted with one or more substituents independently selected from R⁶ and R^{6′}, wherein ring heteroatoms of the 5 to 12 membered heterocycloalkyl and 5 to 12 membered heteroaryl are independently selected from C, N, O, and S; and R² is selected from C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl, wherein C₅₋₁₂cycloalkyl, 5 to 12 membered partially unsaturated heterocycloalkyl, 6 to 12 membered saturated heterocycloalkyl, C₇₋₁₂ aryl, and 5 to 12 membered heteroaryl are substituted with one or more substituents independently selected from R⁷ and R^{7′}, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S.

In embodiments of the subject compound (i.e., compound described herein), or a pharmaceutically acceptable salt or solvate thereof, R¹ is selected from triazolyl, pyrazolyl, pyrrolyl, imidazolyl, phenyl, pyridyl, pyrimidinyl, pyridazinyl, pyrazinyl, and triazinyl, wherein triazolyl, pyrazolyl, pyrrolyl, imidazolyl, phenyl, pyridyl, pyrimidinyl, pyridazinyl, pyrazinyl, and triazinyl are substituted with one or more substituents independently selected from R⁶ and R^{6′}; and R² is selected from triazolyl, pyrazolyl, pyrrolyl, imidazolyl, phenyl, pyridyl, pyrimidinyl, pyridazinyl, pyrazinyl, and triazinyl, wherein triazolyl, pyrazolyl, pyrrolyl, imidazolyl, phenyl, pyridyl, pyrimidinyl, pyridazinyl, pyrazinyl, and triazinyl are substituted with one or more substituents independently selected from R⁷ and R^{7′}.

In embodiments of the subject compound (i.e., compound described herein), or a pharmaceutically acceptable salt or solvate thereof, R¹ is

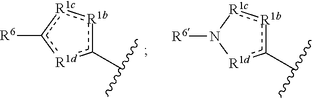

wherein R^{1c} is not N or N(R^{6c″}) when R^{1b} is C(R^{6b}) and R^{1d} is C(R^{6d});

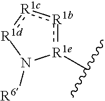

R wherein R¹ is not a substituted pyrrolyl;

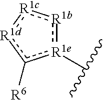

wherein R¹ is not a substituted imidazolyl wherein R^{1d} and R^{1c} are N; or
fluoro-substituted pyrazolyl optionally further substituted with one, two, or three substituents independently selected from R⁶ and R^{6′};
R^{1b} is O, S, N, N(R^{6b″}), C(R^{6b}), or C(R^{6b})(R^{6b′);}
R^{1c} is O, S, N, N(R^{6c″}), C(R^{6c}), or C(R^{6c})(R^{6c′});
R^{1d} is O, S, N, N(R^{6d″}), C(R^{6d}), or C(R^{6d})(R^{6d});
R^{1e} is N or C(R^{6e});
R^{6b}, R^{6b′}, R^{6c}, R^{6c′}, R^{6d}, R^{6d′}, and R^{6e} are independently selected from hydrogen, halogen, —CN, C₁₋₆alkyl, C₂₋₆alkenyl, C₂₋₆alkynyl, C_{3- 10}cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20a}; R^{6b″}, R^{6c″}, and R^{6d″} are independently selected from hydrogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R² is

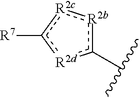

wherein R² is not a substituted imidazolyl wherein R^{2c} and R^{2d} are N;

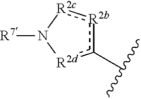

wherein R² is not a substituted pyrazolyl or pyrrolyl;

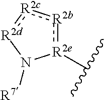

wherein R² is not a substituted pyrrolyl or imidazolyl;

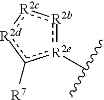

wherein R² is not a substituted imidazolyl wherein R^{2d} and R^{2e} are N, or R^{2b} and R^{2d} are N or NH; or Fluoro-substituted pyrazolyl optionally further substituted with one, two, or three substituents independently selected from R⁷ and R^{7′};
R^{2b} is O, S, N, N(R^{7b″}), C(R^{7b}), or C(R^{7b})(R^{7b′});
R^{2c} is O, S, N, N(R^{7c″}), C(R^{7c}), or C(R^{7c})(R^{7c′});
R^{2d} is O, S, N, N(R^{7d″}), C(R^{7d}), or C(R^{7d})(R^{7d′});
R^{2e} is N or C(R^{7e});
R^{7b}, R^{7b′}, R^{7c}, R^{7c′}, R^{7d}, R^{7d′}, and R^{7e} are independently selected from hydrogen, halogen, —CN, C₁₋₆alkyl, C₂₋₆alkenyl, C₂₋₆alkynyl, C_{3- 10}cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20b};
R^{7b″}, R^{7c″}, and R^{7d″} are independently selected from hydrogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b}; and
each R⁶ and R⁷ are independently selected from halogen, oxo, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁶;
each R^{6′} and R^{7′} are independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀, aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In embodiments of the subject compound (i.e., compound described herein), or a pharmaceutically acceptable salt or solvate thereof, R¹ is

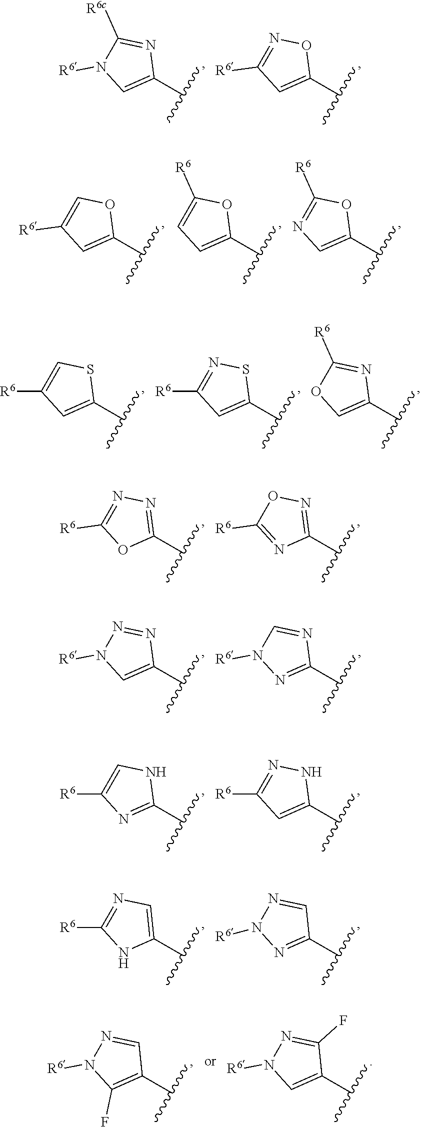

In embodiments of the subject compound (i.e., compound described herein), or a pharmaceutically acceptable salt or solvate thereof, R² is

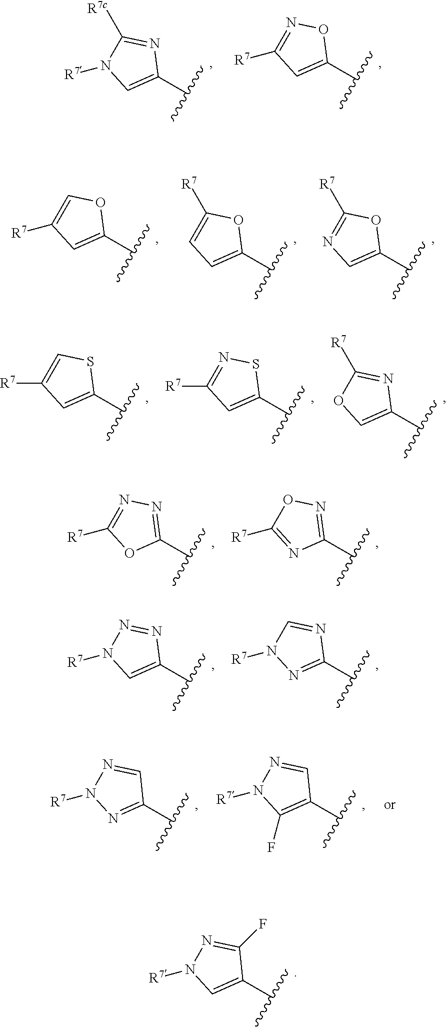

In embodiments of the subject compound (i.e., compound described herein), or a pharmaceutically acceptable salt or solvate thereof, R¹ and R² are selected from

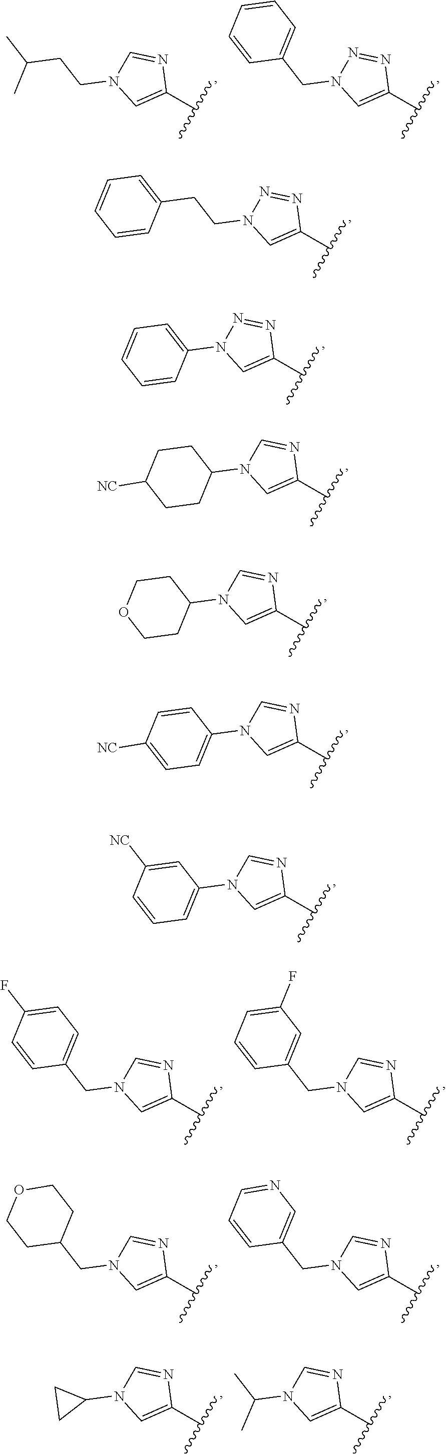

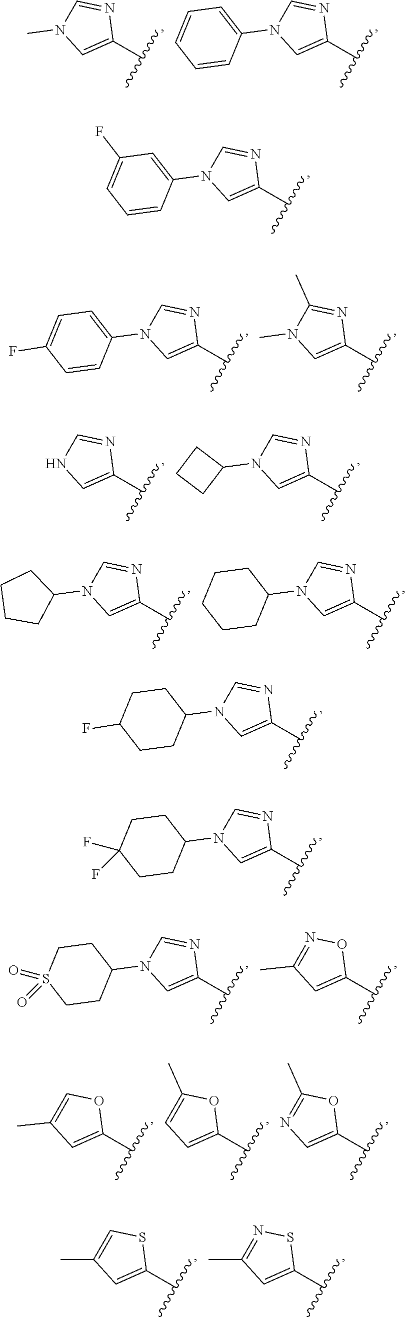

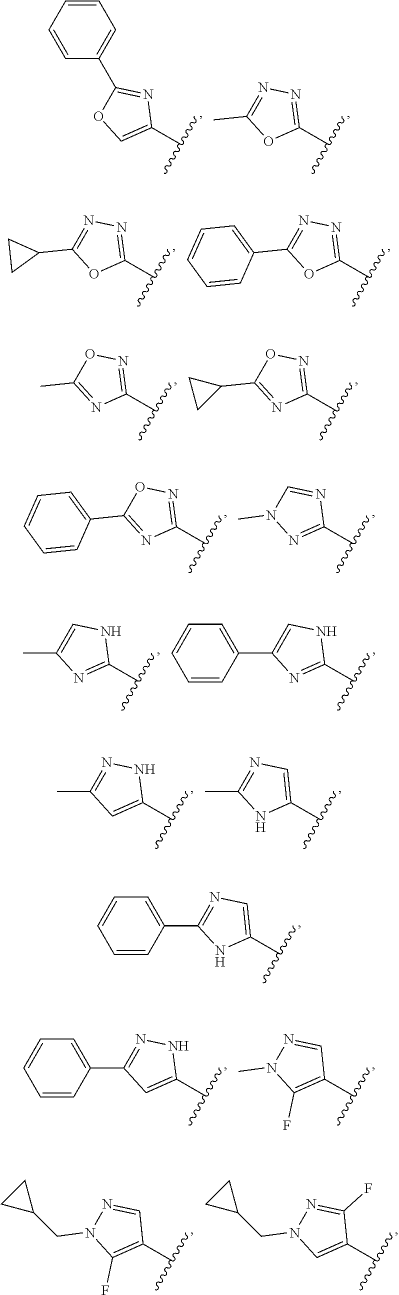

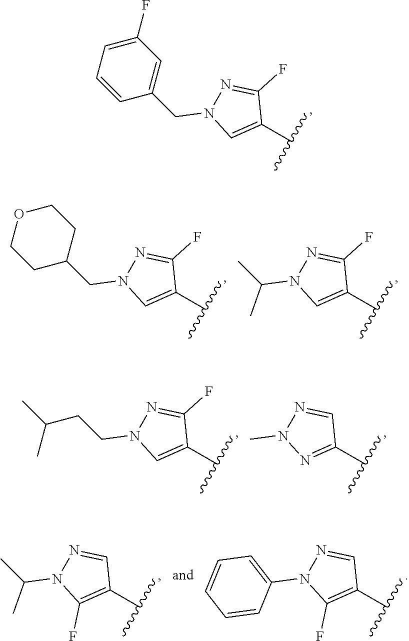

In embodiments of the subject compound (i.e., compound described herein), or a pharmaceutically acceptable salt or solvate thereof, R¹ is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are substituted with one or more substituents independently selected from R⁶ and R^{6′}; and R² is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are substituted with one or more substituents independently selected from R⁷ and R^{7′}.

In embodiments of the subject compound (i.e., compound described herein), or a pharmaceutically acceptable salt or solvate thereof, R¹ is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are substituted with one or more substituents independently selected from R⁶ and R^{6′}; and R² is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are substituted with one or more substituents independently selected from R⁷ and R^{7′}.

In embodiments of the subject compound (i.e., compound described herein), or a pharmaceutically acceptable salt or solvate thereof, R¹ is

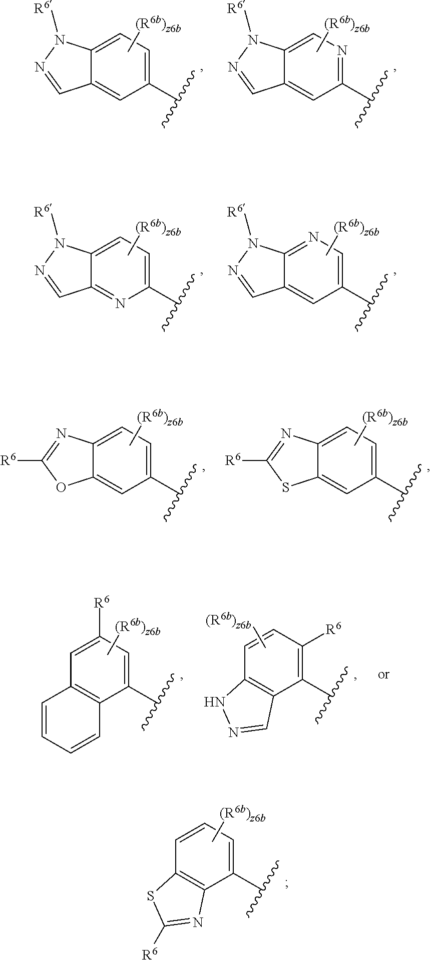

R^{6b} is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
z6b is an integer from 0 to 4;
R² is

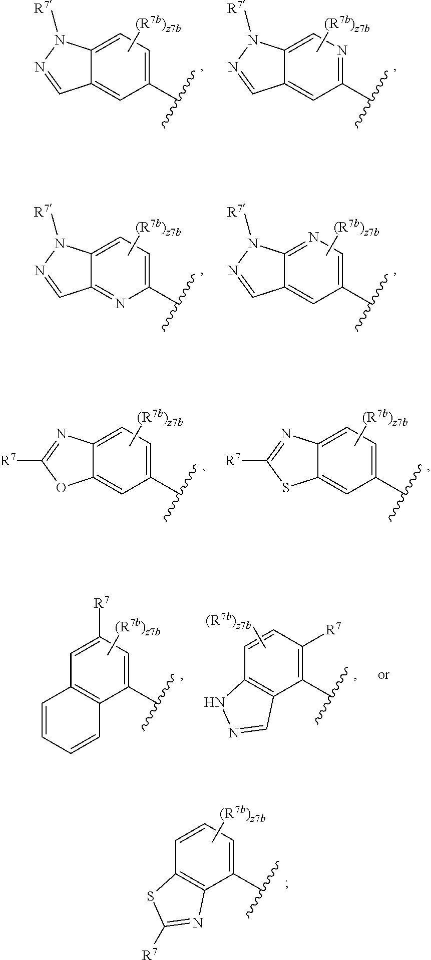

R^{7b} is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b}; and
z7b is an integer from 0 to 4;

In embodiments of the subject compound (i.e., compound described herein), or a pharmaceutically acceptable salt or solvate thereof, R¹ and R² are selected from:

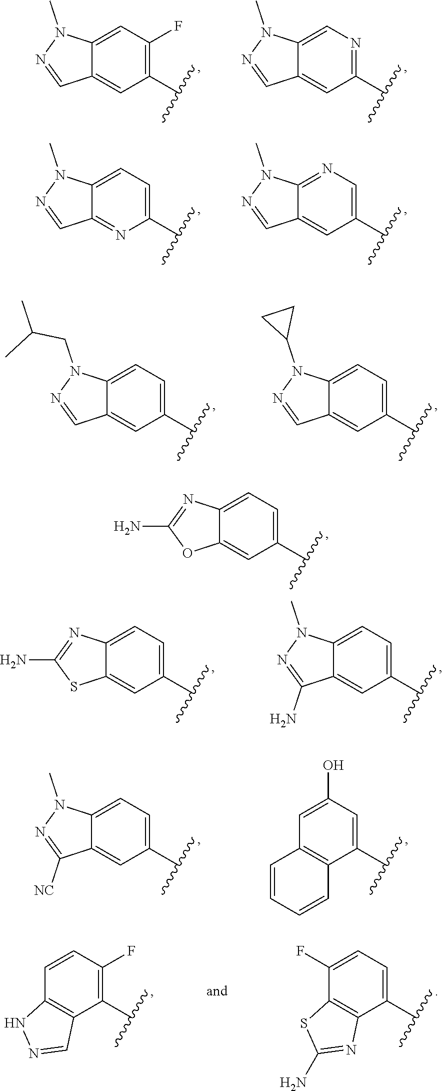

In embodiments of the subject compound (i.e., compound described herein), or a pharmaceutically acceptable salt or solvate thereof, R¹ is pyrazolyl substituted with one or more substituents independently selected from R⁶ and R^{6′}; and R² is pyrazolyl substituted with one or more substituents independently selected from R⁷ and R^{7′}. In embodiments of the subject compound (i.e., compound described herein), or a pharmaceutically acceptable salt or solvate thereof, R¹ is pyrazolyl substituted with one or more substituents independently selected from R^{6′}; and R² is pyrazolyl substituted with one or more substituents independently selected from R^{7′}. In embodiments of the subject compound (i.e., compound described herein), or a pharmaceutically acceptable salt or solvate thereof, R¹ is pyrazolyl substituted with one R^{6′}; and R² is pyrazolyl substituted with one R^{7′}.

In embodiments of the subject compound (i.e., compound described herein), or a pharmaceutically acceptable salt or solvate thereof, R¹ is

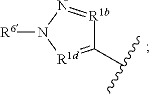

R^{1b} is O, S, N, N(R^{6b″}), C(R^{6b}), or C(R^{6b})(R^{6′});
R^{1d} is O, S, N, N(R^{6d″}), C(R^{6d}), or C(R^{6d})(R^{6d′});
R^{6b}, R^{6b′}, R^{6d}, and R^{6d′} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20a};
R^{6b″} and R^{6d″} are independently selected from hydrogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R² is

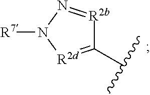

R^{2b} is O, S, N, N(R^{7b″}), C(R^{7b}), or C(R_{7b})(R^{7b′});
R^{2d} is O, S, N, N(R^{7d″}), C(R^{7d}), or C(R^{7d})(R^{7d′});
R^{7b}, R^{7b′}, R^{7d}, and R^{7d∝} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20b};
R^{7b″} and R^{7d″} are independently selected from hydrogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b}; and
R^{6′} and R^{7′} are independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In embodiments of the subject compound (i.e., compound described herein), or a pharmaceutically acceptable salt or solvate thereof, R¹ is

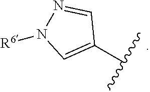

In embodiments of the subject compound (i.e., compound described herein), or a pharmaceutically acceptable salt or solvate thereof, R² is

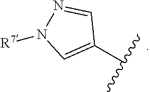

In embodiments of the subject compound (i.e., compound described herein), or a pharmaceutically acceptable salt or solvate thereof, R⁶ is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀ aryl, C₁₋₉heteroaryl, —OR¹², —N(R¹²)(R¹³), —C(O)R¹⁵, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶. In embodiments of the subject compound (i.e., compound described herein), or a pharmaceutically acceptable salt or solvate thereof, R⁶ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In embodiments of the subject compound (i.e., compound described herein), or a pharmaceutically acceptable salt or solvate thereof, R^{6′} is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In embodiments of the subject compound (i.e., compound described herein), or a pharmaceutically acceptable salt or solvate thereof, R⁷ is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀ aryl, C₁₋₉heteroaryl, —OR¹², —N(R¹²)(R¹³), —C(O)R¹⁵, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶. In embodiments of the subject compound (i.e., compound described herein), or a pharmaceutically acceptable salt or solvate thereof, R⁷ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In embodiments of the subject compound (i.e., compound described herein), or a pharmaceutically acceptable salt or solvate thereof, R^{7′} is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In embodiments of the subject compound (i.e., compound described herein), or a pharmaceutically acceptable salt or solvate thereof, R⁶, R^{6′}, R⁷, and R^{7′} are independently selected from C₁₋₆alkyl,

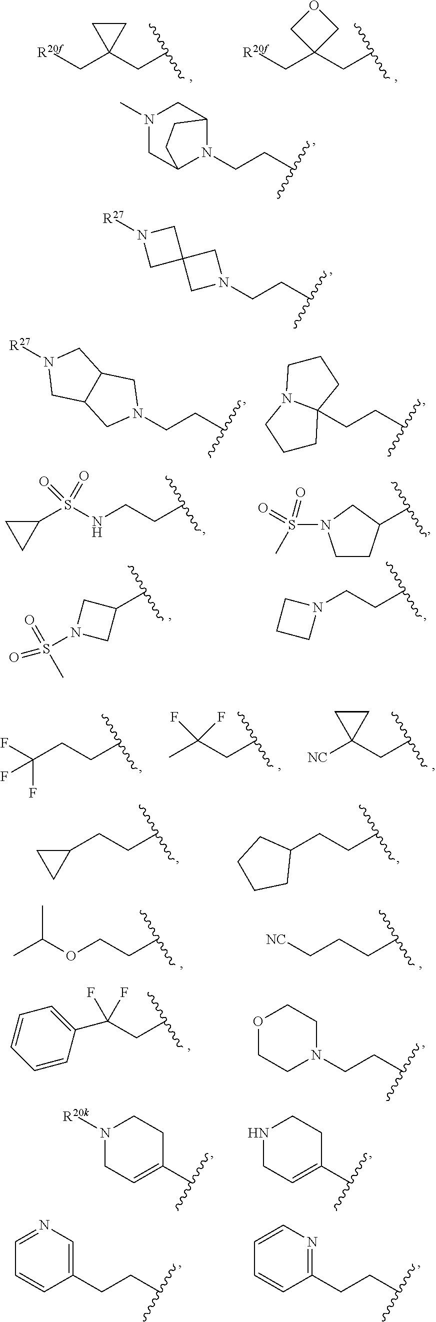

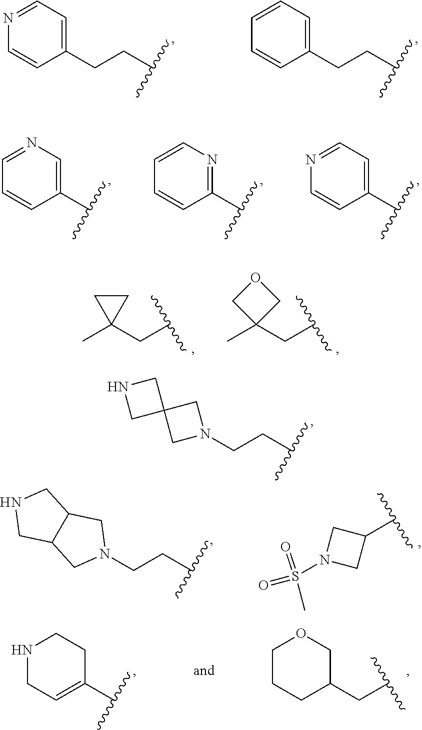

In embodiments of the subject compound (i.e., compound described herein), or a pharmaceutically acceptable salt or solvate thereof, R⁶, R^{6′}, R⁷, and R^{7′} are independently selected from

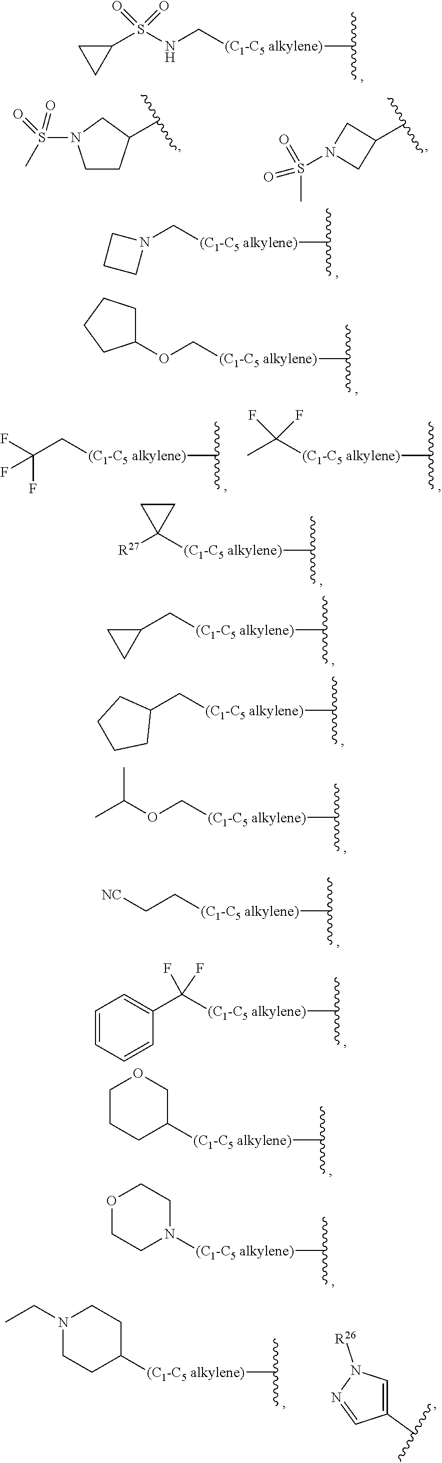

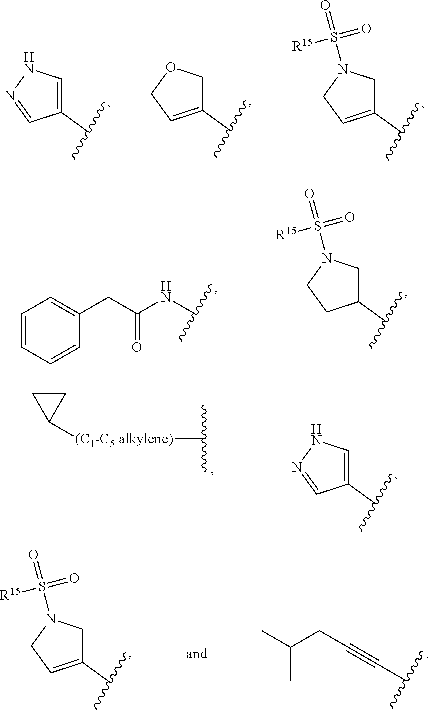

In embodiments of the subject compound (i.e., compound described herein), or a pharmaceutically acceptable salt or solvate thereof. R⁶, R^{6′}, R⁷, and R^{7′} are independently selected from

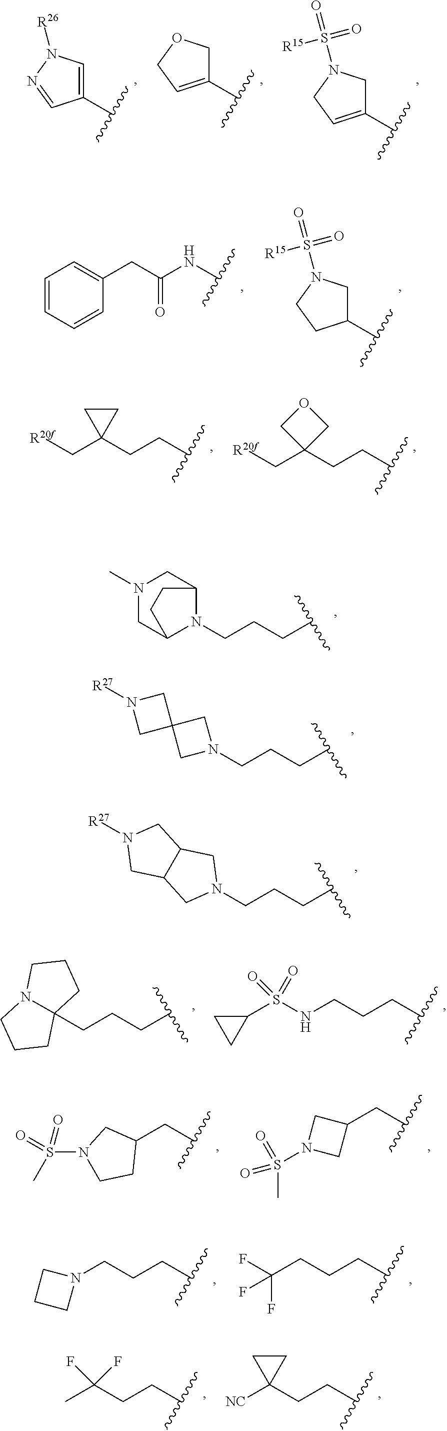

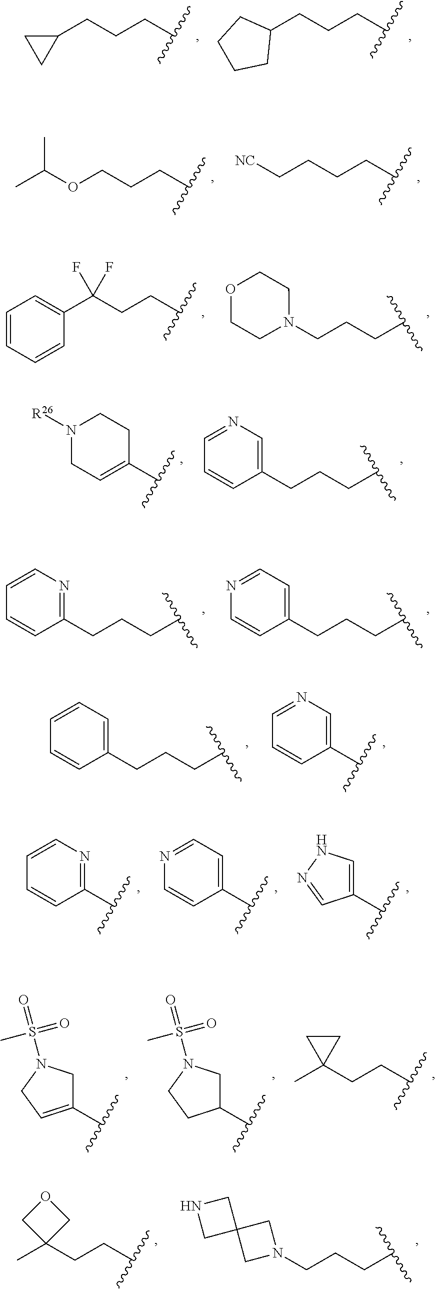

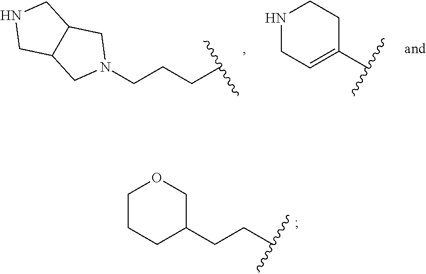 [SMILES: *CCCN1CC2CNCC2C1 ; *C1=CCNCC1 ; *CCC1CCCOC1]

and
R²⁶ is independently selected from hydrogen, halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In embodiments of the subject compound (i.e., compound described herein), or a pharmaceutically acceptable salt or solvate thereof, R⁶, R^{6′}, R⁷, and R^{7′} are independently selected from

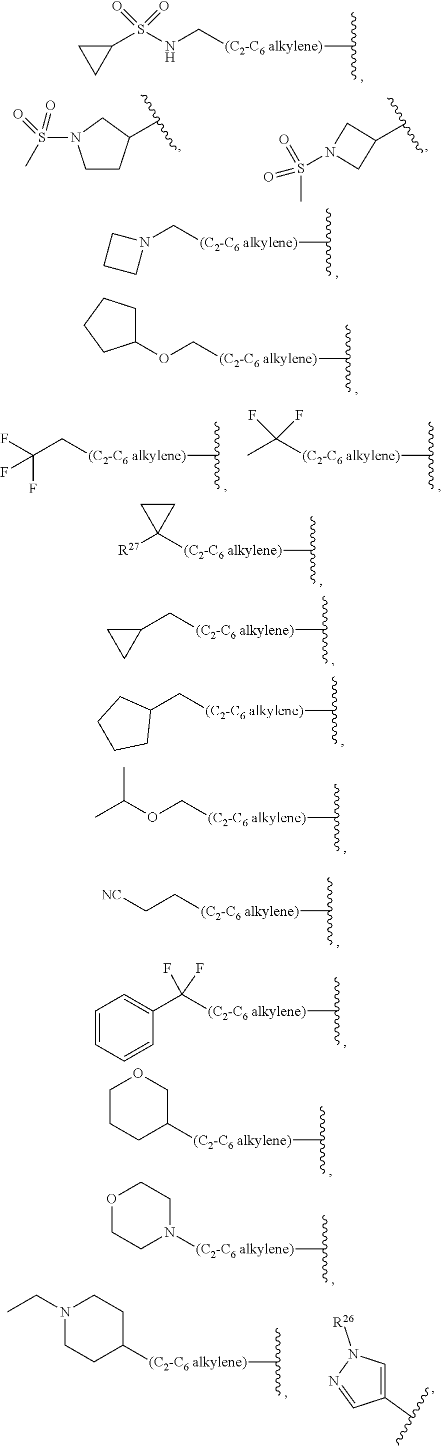

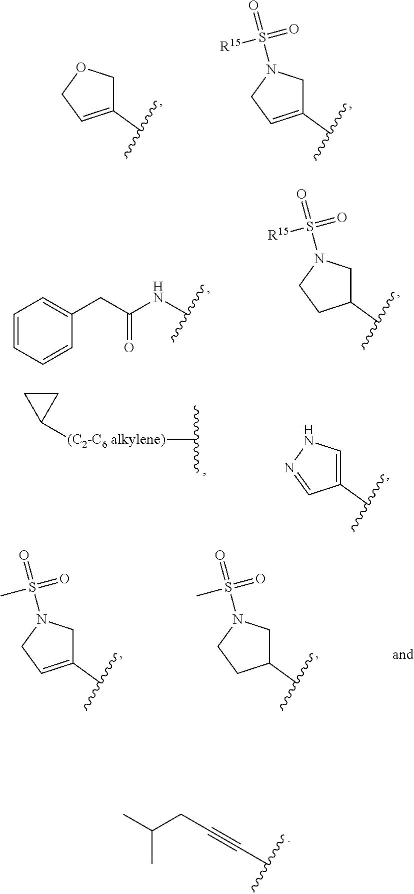

In embodiments of the subject compound (i.e., compound described herein), or a pharmaceutically acceptable salt or solvate thereof, R⁶, R^{6′}, R⁷, and R^{7′} are independently selected from

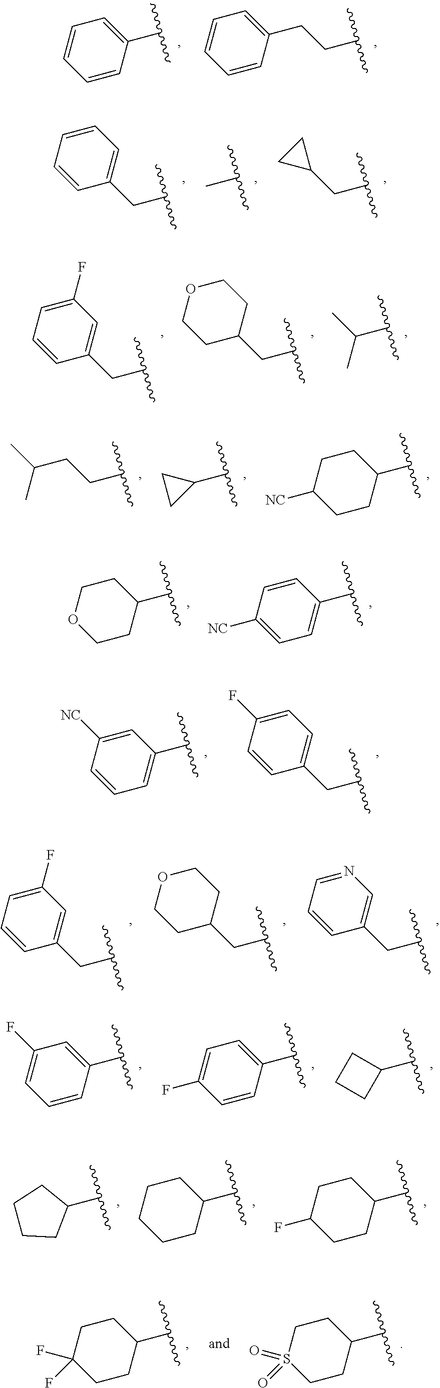

In embodiments of the subject compound (i.e., compound described herein), or a pharmaceutically acceptable salt or solvate thereof, at least one of X, Y, Z, W¹, and W² is N.

In an aspect is provided a compound of Formula (III), or a pharmaceutically acceptable salt or solvate thereof:

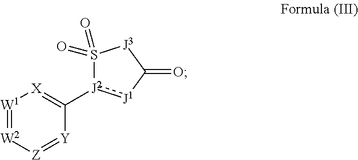

wherein:
either:
W¹ is C(R¹) and W² is C(R^{2a}) or N; or
W¹ is C(R^{1a}) or N, and W² is C(R²);
R¹ is selected from C₁₋₆alkyl, C₂₋₆alkenyl, C₂₋₆alkynyl, C₅₋₁₂cycloalkyl, 5 to 12 membered heterocycloalkyl, C₆₋₁₂aryl, and 5 to 12 membered heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₅₋₁₂cycloalkyl, 5 to 12 membered heterocycloalkyl, C₆₋₁₂aryl, and 5 to 12 membered heteroaryl are optionally substituted with one or more substituents independently selected from R⁶ and R^{6′};
R^{1a} is selected from hydrogen, halogen, —CN, C₁₋₆alkyl, C₂₋₆alkenyl, C₂₋₆alkenyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkenyl are optionally substituted with one, two, or three R^{20a};
R² is selected from C₁₋₆alkyl, C₂₋₆alkenyl, C₂₋₆alkenyl, C₅₋₁₂cycloalkyl, 5 to 12 membered heterocycloalkyl, C₆₋₁₂aryl, and 5 to 12 membered heteroaryl, —OR¹², —SR¹², —N(R¹²)(R¹³), —C(O)OR¹²⁹ —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkenyl, C₅₋₁₂cycloalkyl, 5 to 12 membered heterocycloalkyl, C₆₋₁₂aryl, and 5 to 12 membered heteroaryl are optionally substituted with one or more substituents independently selected from R⁷ and R^{7′};
R^{2a} are independently selected from hydrogen, halogen, —CN, C₁₋₆alkyl, C₂₋₆alkenyl, C₂₋₆alkenyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³)₉—CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20b};
X is N or C(R³);
Y is N or C(R⁴).
Z is N or C(R⁵);
at least one of W¹, W², X, Y, and Z is N;
R³ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20c};
R⁴ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20d};
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20e};
each R⁶ and R⁷ are independently selected from halogen, oxo, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═Ni)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁶;
each R^{6′} and R^{7′} are independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀ aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶;
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉ heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉ heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20l};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted with one, two, or three R^{20m};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R²ⁿ;
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20o};
each R^{20a}, R^{20b}, R^{20c}, R^{20d}, R^{20e}, R^{20f}, R^{20h}, R²⁰ⁱ, R^{20j}, R^{20k}, R^{20l}, R^{20m}, R²⁰ⁿ, and R^{20o} are each independently selected from oxo, —CN, halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl optionally substituted with one or more R²⁶, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl; each R²⁶ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁷;
each R²⁷ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₆ heteroaryl are optionally substituted with one, two, or three R^{20f};
[symbol] indicates a single or double bond such that all valences are satisfied.

In an aspect is provided a compound of Formula (IId-1), or a pharmaceutically acceptable salt or solvate thereof:

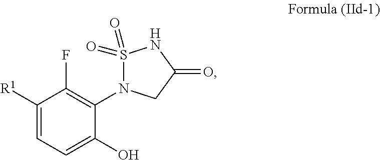

wherein:
R¹ is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are substituted with one or more substituents independently selected from R⁶ and R^{6′}, wherein ring heteroatoms of the 5 to 12 membered heterocycloalkyl and 5 to 12 membered heteroaryl are independently selected from N, O, and S;
each R⁶ is independently selected from halogen, oxo, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —C(NR¹⁴)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R²⁶;
each R^{6′} is independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶;
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉ heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, —CN, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20l};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted with one, two, or three R^{20m};
each R^{20f}, R^{20k}, R^{20l}, and R^{20m} is independently selected from oxo, —CN, halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl optionally substituted with one or more R²⁶, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl;
each R²⁶ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁷; and
each R²⁷ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f}.

In embodiments of the subject compound (i.e., compound described herein), or a pharmaceutically acceptable salt or solvate thereof, R¹ is selected from pyrrolyl, pyrazolyl, imidazolyl, triazolyl, pyrrolinyl, pyridinyl, tetrahydropyridinyl, piperidinyl, and piperazinyl, each of which is substituted with one or more R⁶. In embodiments of the subject compound (i.e., compound described herein), or a pharmaceutically acceptable salt or solvate thereof, R¹ is selected from

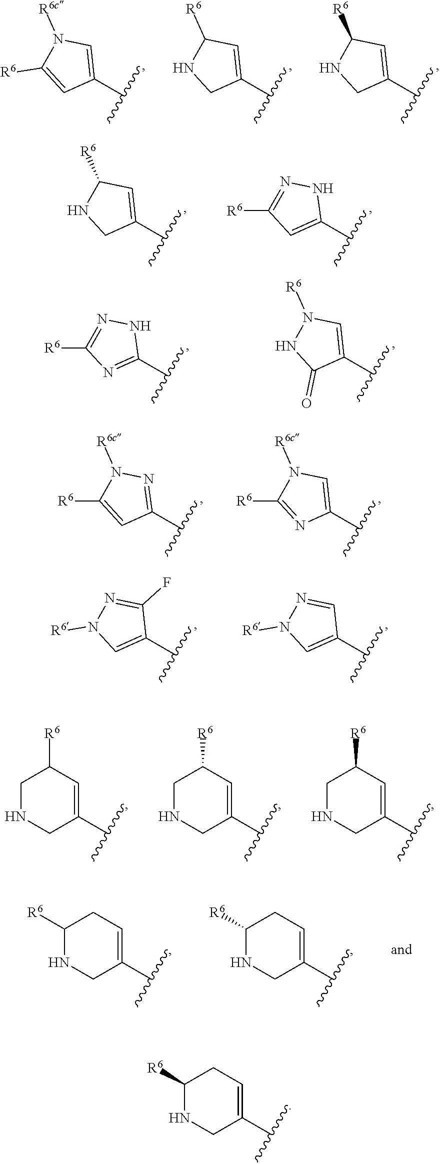

In certain aspects, the present disclosure provides a compound described herein, such as a compound selected from Table 2, or a pharmaceutically acceptable salt or solvate thereof. In an aspect is provided a pharmaceutical composition comprising a compound described herein, or a pharmaceutically acceptable salt or solvate thereof, and a pharmaceutically acceptable excipient.

In an aspect is provided a method of treating cancer in a subject in need thereof, comprising administering to the subject a therapeutically effective amount of a subject compound (i.e., compound described herein) described herein, or a pharmaceutically acceptable salt or solvate thereof. The subject may suffer from a solid tumor or a liquid cancer (e.g., cancer of the blood or bone marrow or lymphoid nodes, including leukemia, lymphoma and myeloma).

In an aspect is provided a method of potentiating immunity of a cell, comprising: contacting the cell with a compound described herein, thereby potentiating immunity of the cell, wherein the cell comprises (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen.

In an aspect is provided a method of potentiating immunity of a cell, comprising: (a) contacting the cell with a compound described herein; and (b) introducing to the cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen, thereby potentiating immunity of the cell. In some embodiments, the step (a) is performed prior to, concurrent with, or subsequent to (b). In some embodiments, the cell retains expression or activity of PTPN2 prior to (a). In some embodiments, the cell is a lymphoid cell. In some embodiments, the subject method further comprises administering the cell to a subject in need thereof. In some embodiments, the subject method further comprises administering a compound described herein to the subject prior to, concurrent with, or subsequent to the administering the cell. In some embodiments, prior to the administering a compound described herein, a cell of the subject exhibits expression or activity of PTPN2.

In an aspect is provided a method of potentiating immunity of a subject in need thereof, comprising: administering a lymphoid cell to the subject, thereby potentiating immunity of the subject, wherein expression or activity of PTPN2 in the lymphoid cell is downregulated by a subject compound (i.e., compound described herein) disclosed herein. In some embodiments, the subject method comprises transiently downregulating the expression or activity of PTPN2 in the lymphoid cell. In some embodiments, prior to the transiently downregulating, the lymphoid cell exhibits expression or activity of PTPN2. In some embodiments, the transiently downregulating is performed once. In some embodiments, the transiently downregulating is performed intermittently for two or more times. In some embodiments, the transiently downregulating comprises introducing a compound described herein to the cell. In some embodiments, the lymphoid cell comprises (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In some embodiments, the subject method further comprises administering a compound described herein to the subject prior to, concurrent with, or subsequent to the administering the lymphoid cell. In some embodiments, prior to the administering the compound described herein, a cell of the subject exhibits expression or activity of PTPN2.

In an aspect is provided a method of potentiating immunity of a subject in need thereof, comprising: (a) selecting the subject that exhibits expression or activity of PTPN2; and (b) downregulating expression or activity of PTPN2 in a cell of the subject by a subject compound (i.e., compound described herein) disclosed herein, thereby potentiating immunity of the subject. In some embodiments, the step (b) is performed in vivo. In some embodiments, the step (b) is performed ex vivo. In some embodiments, the subject method further comprises administering the cell to the subject prior to, concurrent with, or subsequent to the downregulating. In some embodiments, the downregulating comprises introducing a compound described herein to the cell. In some embodiments, the downregulating comprises transiently downregulating the expression or activity of PTPN2. In some embodiments, the transiently downregulating is performed once. In some embodiments, the cell of the subject is a lymphoid cell comprising (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In some embodiments of the subject method, the cell of the subject does not exhibit a mutation of (i) a first gene encoding PTPN2 or (ii) a second gene operatively linked to PTPN2, wherein the mutation inhibits the expression and/or activity of PTPN2. In some embodiments of the subject method, the selecting comprises performing a nucleic acid assay using at least a portion of a genome or transcriptome of the cell of the subject to detect the mutation. In some embodiments of the subject method, the selecting comprises performing a protein assay to detect a functionally active PTPN2 or a functionally inactive PTPN2.

In an aspect is provided a method of potentiating immunity of a subject in need thereof, comprising: administering a lymphoid cell to the subject; and administering a compound described herein to the subject, thereby potentiating immunity of the subject. In some embodiments of the subject method, the administering the compound described herein is performed prior to, concurrent with, or subsequent to the administering the lymphoid cell. In some embodiments of the subject method, the administering the compound described herein is performed separately from the administering the lymphoid cell. In some embodiments of the subject method, prior to the administering the compound described herein, a cell of the subject exhibits expression or activity of PTPN2. In an aspect is provided a method of potentiating anti-tumor or anti-cancer immunity of a subject in need thereof, comprising: (a) contacting a lymphoid cell of the subject with a compound described herein, thereby potentiating the anti-tumor or anti-cancer immunity of the subject.

In an aspect is provided a method of treating tumor or cancer of a subject in need thereof, comprising: (a) contacting a lymphoid cell of the subject with a compound described herein, thereby treating the tumor or cancer of the subject. In some embodiments of the subject method, the contacting is performed in vivo. In some embodiments of the subject method, the contacting is performed ex vivo, and subsequently followed by introducing the lymphoid cell to the subject. In some embodiments, the subject method further comprises administering the lymphoid cell to the subject prior to, concurrent with, or subsequent to the contacting. In some embodiments, the subject method further comprises (b) introducing to the lymphoid cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In some embodiments of the subject method, (a) is performed prior to, concurrent with, or subsequent to (b).

In an aspect is provided a method of potentiating anti-tumor or anti-cancer immunity of a subject in need thereof, comprising: (a) downregulating expression or activity of PTPN2 in a lymphoid cell of the subject, thereby potentiating the anti-tumor or anti-cancer immunity of the subject.

In an aspect is provided a method of treating tumor or cancer of a subject in need thereof, comprising: (a) downregulating expression or activity of PTPN2 in a lymphoid cell of the subject, thereby treating the tumor or cancer of the subject. In some embodiments of the subject method, the downregulating is performed in vivo. In some embodiments of the subject method, the downregulating is performed ex vivo, and subsequently followed by introducing the lymphoid cell to the subject. In some embodiments, the subject method further comprises administering the lymphoid cell to the subject prior to, concurrent with, or subsequent to the downregulating. In some embodiments, the downregulating comprises introducing a compound described herein to the lymphoid cell. In some embodiments, the downregulating comprises transiently downregulating the expression or activity of PTPN2. In some embodiments, the transiently downregulating is performed once. In some embodiments of the subject method, the transiently downregulating is performed intermittently for two or more times. In some embodiments, the subject method further comprises (b) introducing to the lymphoid cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In some embodiments of the subject method, (a) is performed prior to, concurrent with, or subsequent to (b).

In an aspect is provided a method of increasing efficacy or reducing side effect of a cell therapy for a subject in need thereof, comprising: (a) administering to the subject a cell comprising a chimeric antigen receptor (CAR) sequence encoding a CAR, wherein the CAR comprises an antigen-binding domain and an intracellular signaling domain, wherein the intracellular signaling domain is minimally required for activation of the CAR upon binding to an antigen; and (b) administering a compound described herein to the subject prior to, concurrent with, or subsequent to (a). In some embodiments, the cell retains expression or activity of PTPN2 prior to (b). In some embodiments, the cell is a lymphoid cell. In some embodiments, prior to the administering the compound described herein, a cell of the subject exhibits expression or activity of PTPN2.

In an aspect is provided a method of increasing efficacy or reducing side effect of a cell therapy for a subject in need thereof, comprising: (a) administering to the subject a sub-therapeutic amount of a cell comprising a chimeric antigen receptor (CAR) sequence encoding a CAR, (b) administering a compound described herein to the subject prior to, concurrent with, or subsequent to (a). In some embodiments, the cell retains expression or activity of PTPN2 prior to (b). In some embodiments, the cell is a lymphoid cell. In some embodiments, prior to the administering the compound described herein, a cell of the subject exhibits expression or activity of PTPN2. In some embodiments, the compound described herein reduces PTPN2 signaling in a cell of the subject. In some embodiments, the compound described herein does not regulate site-specific recombination of a gene encoding PTPN2. In some embodiments, the compound described herein does not affect editing of (i) the gene encoding PTPN2 or (ii) an additional gene operatively linked to PTPN2. In some embodiments, the compound described herein is configured to bind PTPN2. In some embodiments, the compound described herein exhibits binding specificity to PTPN2 in comparison to other tyrosine phosphatases. In some embodiments, the compound described herein exhibits IC₅₀ of less than or equal to 5 μM for PTPN2.

In some embodiments, any of the subject method further comprises monitoring, concurrent with or subsequent to the administration of the compound described herein and/or the lymphoid cell, one or more health parameters of the subject selected from the group consisting of: temperature, wheezing, sweating, fatigue, weight, insomnia, diarrhea, infections, and mental disorders.

In some embodiments, any of the subject method further comprises detecting, concurrent with or subsequent to the administration of the compound described herein of the lymphoid cell, one or more inflammatory biomarkers selected from the group consisting of: antibodies, cytokines, radicals, and coagulation factors. In some embodiments of the subject method, the cytokines comprise IL-1, IL-6, TNF-α, IL-10, or IL-1RA. In some embodiments of the subject method, the cell of the subject comprises a diseased cell. In some embodiments of the subject method, the diseased cell is a tumor cell or a cancer cell. In some embodiments of the subject method, the cell of the subject comprises a lymphoid cell. In some embodiments of the subject method, the lymphoid cell is selected from the group consisting of: T cell, B cell, NK cell, KHYG cell, T helper cell, regulatory T cell, memory T cell, tumor infiltration T cell (TIL), antigen presenting cell, and dendritic cell. In some embodiments of the subject method, the lymphoid cell is selected from the group consisting of: a CD4+ T cell, a CD8+ T cell, and a CD4+ and CD8+ T cell.

In some embodiments of any of the subject method, the subject suffers from a cancer selected from cancer of bladder, bone, brain, breast, cervical, colon, lung, esophagus, head and neck, ovary, prostate, uterus, stomach, skin, and renal tissue.

In some embodiments of any of the applicable subject method, the step of (1) the contacting the cell with a compound described herein, (2) the administering the lymphoid cell to the subject, (3) the downregulating the expression or activity of PTPN2 in the cell of the subject, (4) the administering the compound described herein to the subject, (5) the contacting the lymphoid cell of the subject with the compound described herein, and/or (6) the downregulating the expression or activity of PTPN2 in the lymphoid cell of the subject is performed prior to, concurrent with, or subsequent to an administration of another agent (second agent) or therapy to the subject.

In some embodiments of the aforementioned subject method, the second agent can be selected from the group including without limitation a chemotherapeutic agent, a radioactive agent, a small molecule agent targeting a tumor marker, an antigen-binding agent specifically binding to a tumor marker, and an immune modulator. In some embodiments, the second agent is a checkpoint inhibitor. In some embodiments, the second agent is an inhibitor of PD1, PD-L1, LAG3, CTLA4, CD160, BTLA, LAIR1, TIM3,2B4, CD93, OX40, Siglec-15, and TIGIT. In some embodiments, the second agent is an inhibitor of IDO or mTOR.

In some embodiments, a second therapy comprising stem cells or lymphoid cells can be used conjunctively with a subject compound (i.e., compound described herein) disclosed herein.

In some embodiments of a subject method, the TFP utilized in a subject cell comprises a TCR subunit that comprises (1) a TCR extracellular domain capable of specific binding to an antigen, and (2) an intracellular signaling domain, wherein the TFP forms a TCR complex. In some embodiments, the TCR extracellular domain comprises element (1) an antigen binding domain capable of specific binding to the antigen, and element (2) an extracellular domain or portion thereof of a protein selected from the group consisting of a TCR alpha chain, a TCR beta chain, a CD3 epsilon TCR subunit, a CD3 gamma TCR subunit, a CD3 delta TCR, wherein elements (1) and (2) are operatively linked together. In some embodiments, the TCR intracellular domain comprising a stimulatory domain from an intracellular signaling domain of epsilon chain, delta chain, and/or a gamma chain of cluster of differentiation 3 (CD3). In some embodiments, the TCR intracellular domain comprising a stimulatory domain from an intracellular signaling domain of TCR alpha, or from an intracellular signaling domain of TCR beta. In some embodiments, the TFP comprises a transmembrane domain including a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, and CD154.

In some embodiments, the TFP comprises a costimulatory domain. In some embodiments of the subject method, the costimulatory domain of the TFP is selected from the group consisting of: a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-113, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In some embodiments, a CAR utilized in a subject method comprises an antigen-binding domain and an intracellular signaling domain. In some embodiments, the intracellular signaling domain of the CAR comprises a primary signaling domain and/or a costimulatory signaling domain, wherein the primary signaling domain comprises a functional signaling domain of a protein chosen from CD3 zeta, CD3 gamma, CD3 delta, CD3 epsilon, common FcR gamma (FCERIG), FcR beta (Fc Epsilon Rib), CD79a, CD79b, Fcgamma R11a, DAP10, or DAP12.

In some embodiments, the intracellular signaling domain of the CAR comprises a costimulatory signaling domain that comprises a functional signaling domain of a protein selected from the group consisting of CD27, CD28, 4-1BB (CD137), OX40, CD30, CD40, PD-1, ICOS, lymphocyte function-associated antigen- 1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In some embodiments, the intracellular signaling domain of the CAR comprises a primary signaling domain and/or a costimulatory signaling domain, wherein the primary signaling domain and/or the costimulatory signaling domain is minimally required for activation of the CAR upon binding to an antigen. In some embodiments of the subject method, the CAR is a first generation CAR in which the primary signaling domain is a member selected from the group consisting of CD3zeta, CD28, 4-1BB, OX40, DAP10, ICOS, and a variant thereof. In some embodiments of the subject method, the CAR is a second generation CAR in which (i) the primary signaling domain is a member selected from the group consisting of CD3zeta, CD28, 4-1BB, OX40, DAP10, ICOS, and a variant thereof, and (ii) the co-stimulatory signaling domain is a different member selected from the group consisting of CD3zeta, CD28, 4-1BB, OX40, DAP10, ICOS, and a variant thereof.

In some embodiments of a subject method, the antigen to which a subject cell binds is a tumor antigen or cancer antigen a tumor antigen selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY—BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1. In some embodiments of the subject method, the antigen comprises a neoantigen encoded by a tumor-specific mutated gene.

In some embodiments, a subject method utilizing a subject compound (i.e., compound described herein) disclosed herein is effective in reducing the side effect comprising cytokine release syndrome (CRS), inflammatory disorder, or autoimmune disorder.

In an aspect is provided a modified cell that has been exposed to a subject compound (i.e., compound described herein) disclosed herein. In another aspect, the modified cell comprises a compound described herein.

In a separate aspect is provided a modified cell comprising (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen, wherein expression or activity of PTPN2 in the cell is downregulated, to potentiate immunity of the modified cell.

In some embodiments of the subject modified cell, the modified cell exhibits a mutation of (i) a first gene encoding PTPN2 or (ii) a second gene operatively linked to PTPN2, wherein the mutation inhibits the expression and/or activity of PTPN2. In some embodiments, the expression or activity of PTPN2 is transiently downregulated. In some embodiments, the expression or activity of PTPN2 is downregulated by a compound described herein. In some embodiments, the compound described herein does not regulate site-specific recombination of a gene encoding PTPN2. In some embodiments, the compound described herein does not affect editing of (i) the gene encoding PTPN2 or (ii) an additional gene operatively linked to PTPN2. In some embodiments of the subject modified cell, the compound described herein is configured to bind PTPN2, and in some embodiment, the compound exhibits specific binding to PTPN2 in comparison to other tyrosine phosphatases. In some embodiments, the compound described herein exhibits IC₅₀ of less than or equal to 5 μM for PTPN2. In some embodiments, a subject compound (i.e., compound described herein) exhibits selective inhibition of PTPN2 relative to another phosphatase such as PTPN1B. In some embodiments, a subject compound (i.e., compound described herein) binds to and inhibits activities of PTPN2 and PTPN1B.

In some embodiments of a subject modified cell, the TFP utilized comprises a TCR subunit that comprises (1) a TCR extracellular domain capable of specific binding to the antigen, and (2) an intracellular signaling domain, wherein the TFP forms a TCR complex. In some embodiments, TCR extracellular domain comprises element (1) an antigen binding domain capable of specific binding to the antigen, and element (2) an extracellular domain or portion thereof of a protein selected from the group consisting of a TCR alpha chain, a TCR beta chain, a CD3 epsilon TCR subunit, a CD3 gamma TCR subunit, a CD3 delta TCR, wherein elements (1) and (2) are operatively linked together. In some embodiments, the TCR intracellular domain comprising a stimulatory domain from an intracellular signaling domain of epsilon chain, delta chain, and/or a gamma chain of cluster of differentiation 3 (CD3). In some embodiments, the TCR intracellular domain comprising a stimulatory domain from an intracellular signaling domain of TCR alpha, or from an intracellular signaling domain of TCR beta. In some embodiments, the TFP comprises a transmembrane domain including a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, and CD154.

In some embodiments, the TFP comprises a costimulatory domain. In some embodiments, the costimulatory domain of the TFP is selected from the group consisting of: a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAIMF1, CD150, IPO-3), BLAME (SLAMF8), SELPLG (CD162), LTBR, LAT, GADS, SLP-76, PAG/Cbp, NKp44, NKp30, NKp46, and NKG2D.

A subject modified cell can be a modified lymphoid cell. In some embodiments, the modified lymphoid cell is a variant of a member selected from the group consisting of: a T cell, B cell, NK cell, KHYG cell, T helper cell, regulatory T cell, memory T cell, tumor infiltration T cell (TIL), antigen presenting cell, and dendritic cell. In some embodiments, the modified lymphoid cell is a variant of a member selected from the group consisting of: a CD4+ T cell, a CD8+ T cell, and a CD4+ and CD8+ T cell.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Unless defined otherwise, all technical and scientific terms used herein have the same meaning as is commonly understood by one of skill in the art to which this invention belongs.

The terms “a” and “an” refer to one or to more than one (i.e., to at least one) of the grammatical object of the article. By way of example, “an element” means one element or more than one element.

“About” as used herein when referring to a measurable value such as an amount, a duration, and the like, is meant to encompass variations of ±10% of a stated number or value.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, primers, cell-free DNA (cfDNA), and circulating tumor DNA (ctDNA). A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

A “nucleotide probe” or “probe” refers to a polynucleotide used for detecting or identifying its corresponding target polynucleotide in a hybridization reaction.

As used herein, “expression” refers to the process by which a polynucleotide is transcribed into mRNA and/or the process by which the transcribed mRNA (also referred to as a “transcript”) is subsequently translated into peptides, polypeptides, or proteins. The transcripts and the encoded polypeptides are collectedly referred to as “gene product.” If the polynucleotide is derived from genomic DNA, expression may include splicing of the mRNA in a eukaryotic cell. The level of expression (or alternatively, the “expression level”) of a PTPN2 gene can be determined, for example, by determining the level of PTPN2polynucleotides, polypeptides or gene products.

“Aberrantly expressed” or “aberrant expression” as applied to a nucleotide sequence (e.g., a gene) or polypeptide sequence in a subject, refers to the aberrant production of the mRNA transcribed and/or translated from the nucleotide sequence or the protein product encoded by the nucleotide sequence. A differentially expressed sequence may be overexpressed (or aberrantly high expression) or underexpressed (or aberrantly low expression) as compared to the expression level of a reference sample (i.e., a reference level). As used herein, overexpression is an increase in expression can be at least 1.25 fold, or alternatively, at least 1 fold, or alternatively, at least 2 fold, or alternatively, at least 3 fold, or alternatively, at least 4 fold, or alternatively, at least 10 fold expression over that detected in a reference sample. As used herein, underexpression is a reduction in expression can be at least 1.25 fold, or alternatively, at least 1 fold, or alternatively, at least 2 fold, or alternatively, at least 3 fold, or alternatively, at least 4 fold, or alternatively, at least 10 fold expression under that detected in a reference sample. Underexpression also encompasses absence of expression of a particular sequence as evidenced by the absence of detectable expression in a test subject when compared to a reference sample.

“Signal transduction” is a process during which stimulatory or inhibitory signals are transmitted into and within a cell to elicit an intracellular response. A molecule can mediate its signaling effect via direct or indirect interaction with downstream molecules of the same pathway or related pathway(s). For instance, PTPN2signaling can involve a host of downstream molecules including but not limited to one or more of the following proteins: PI3-kinase and AKT.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

A “control” or “control sample” is an alternative sample or subject used in an experiment for comparison purpose.

The term “reference level” refers to a control level used to evaluate a test level. In some examples, a reference level may be a control. For example, a biomarker may be considered to be underexpressed when the expression level of that biomarker is lower than a reference level. The reference level can be determined by a plurality of methods, provided that the resulting reference level accurately provides a level of a biomarker above which exists a first group of subjects having a different probability of exhibiting a clinically beneficial response to treatment with a PTPN2 inhibitor than that of a second group of patients having levels of the biomarker below the reference level. The reference level may be determined, for example, by measuring the level of expression of a biomarker in tumorous or non-tumorous cancer cells from the same tissue as the tissue of the cancer cells to be tested. In some examples, the reference level may be a level of a biomarker determined in vitro. A reference level may be determined by comparison of the level of a biomarker in populations of subjects having the same cancer. Two or more separate groups of subjects may be determined by identification of subsets of populations of the cohort that have the same or similar levels of a biomarker. Determination of a reference level can then be made based on a level that distinguishes these separate groups. A reference level may be a single number, equally applicable to every subject, or a reference level can vary according to specific subpopulations of subjects. For example, older men may have a different reference level than younger men for the same cancer, and women may have a different reference level than men for the same cancer. Furthermore, the reference level may be some level determined for each subject individually. For example, the reference level may be a ratio of a biomarker level in a cancer cell of a subject relative to the biomarker level in a normal cell within the same subject. In some embodiments, a reference level is a numerical range of gene expression that is obtained from a statistical sampling from a population of individuals having cancer. The sensitivity of the individuals having cancer to treatment with a PTPN2 inhibitor may be known. In certain embodiments, the reference level is derived by comparing gene expression to a control gene that is expressed in the same cellular environment at relatively stable levels (e.g. a housekeeping gene such as an actin). Comparison to a reference level may be a qualitative assessment or a quantitative determination.

The terms “determining,” “measuring,” “evaluating,” “assessing,” “assaying,” “testing,” and “analyzing” are used interchangeably herein to refer to any form of measurement, and include determining if an analyte is present or not (e.g., detection). These terms can include both quantitative and/or qualitative determinations. Assessing may be relative or absolute. A relative amount could be, for example, high, medium or low. An absolute amount could reflect the measured strength of a signal or the translation of this signal strength into another quantitative format, such as micrograms/mL. “Detecting the presence of” can include determining the amount of something present, as well as determining whether it is present or absent.

The terms “antagonist” and “inhibitor” are used interchangeably, and they refer to a compound, or a biological molecule having the ability to effect inhibition of a biological function of a target protein (e.g., PTPN2), whether by inhibiting the activity or expression of the target protein. Accordingly, the terms “antagonist” and “inhibitors” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. Alternatively or in addition to, an activity of a target protein may involve interaction (e.g., binding) between the target protein and a substrate of the target protein, and the terms “antagonist” and “inhibitors” can refer to a compound having the ability to interact with (e.g., bind to) the subject of the target protein, to indirectly inhibit the biological activity of the target protein. In some cases, such compound may bind both the target protein and one or more kinds of the substrate. A preferred biological activity inhibited by an antagonist is associated with the development, growth, maintenance, or spread of a cancer or a tumor.

The term “cell proliferation” refers to a phenomenon by which the cell number has changed as a result of division. This term also encompasses cell growth by which the cell morphology has changed (e.g., increased in size) consistent with a proliferative signal.

The terms “administer,” “administering,” “administration,” and derivatives thereof refer to the methods that may be used to enable delivery of agents or compositions to the desired site of biological action. These methods include, but are not limited to parenteral administration (e.g., intravenous, subcutaneous, intraperitoneal, intramuscular, intravascular, intrathecal, intranasal, intravitreal, infusion and local injection), transmucosal injection, oral administration, administration as a suppository, and topical administration. Administration is by any route, including parenteral. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transplantation, etc. One skilled in the art will know of additional methods for administering a therapeutically effective amount of a composition of the present disclosure for preventing or relieving one or more symptoms associated with a disease.

The term “systemic administration” refers to administration of agents or compositions such that the agents or compositions become distributed in a subject's body. The distribution of the agents or compositions throughout the subject's body may be an even distribution. Alternatively, the distribution may be preferential, resulting in a higher localization of the agents or compositions in one or more desired sites. A desired site may be the blood or another site that is reachable by the vascular system. Non-limiting examples of systemic routes of administration include administration by (1) introducing the agent directly into the vascular system or (2) oral, pulmonary, or intramuscular administration wherein the agent is adsorbed, enters the vascular system, and is carried to one or more desired site(s) of action via the blood. By contrast, “non-systemic administration” refers to administration of agents or compositions such that the agents or compositions are administered locally to the target site of interest of a subject's body to effect primarily a local effect.

The terms “co-administration,” “administered in combination with,” and their grammatical equivalents, encompass administration of two or more agents to a subject so that both agents and/or their metabolites can assert their respective functions. Co-administration includes simultaneous administration in separate compositions, administration at different times in separate compositions, or administration in a composition in which both agents are present.

The term “effective amount” refers to that amount of a compound described herein that is sufficient to effect the intended application including but not limited to stimulating or prolonging anti-tumor immunity, or disease treatment, as defined below. The effective amount may vary depending upon the intended application (in vitro, ex vivo, or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., cell death or cell activation. The specific dose will vary depending on the particular compounds chosen, the dosing regimen to be followed, whether it is administered in combination with other compounds, the tissue to which it is administered, and the physical delivery system in which it is carried.

As used herein, the terms “treatment”, “treating”, “palliating” and “ameliorating” are used interchangeably. These terms refer to an approach for obtaining beneficial or desired results including, but are not limited to, therapeutic benefit and/or a prophylactic benefit. By therapeutic benefit is meant eradication or amelioration of the underlying disorder being treated (e.g., squamous cell carcinoma). Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compositions may be administered to a patient at risk of developing a particular disease, or to a patient reporting one or more of the physiological symptoms of a disease, even though a diagnosis of this disease may not have been made.

A “therapeutic effect,” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “subject” includes, but is not limited to, humans of any age group, e.g., a pediatric subject (e.g., infant, child or adolescent) or adult subject (e.g., young adult, middle-aged adult or senior adult)) and/or other primates (e.g., cynomolgus monkeys or rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. The methods described herein can be useful in both human therapeutics and veterinary applications. In some embodiments, the subject is a mammal, and in some embodiments, the subject is human.

The term “in vivo” refers to an event that takes place in a subject's body.

The term “ex vivo” refers to an event that first takes place outside of the subject's body for a subsequent in vivo application into a subject's body. For example, an ex vivo preparation may involve preparation of cells outside of a subject's body for the purpose of introduction of the prepared cells into the same or a different subject's body.

The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

The term “downregulating PTPN2 activity”, as used herein, refers to slowing, reducing, altering, inhibiting, as well as completely eliminating and/or preventing PTPN2 activity.

The term “effector function” refers to a specialized function of a cell. Effector function of a T-cell, for example, may be cytolytic activity or helper activity including the secretion of cytokines. Thus the term “intracellular signaling domain” refers to the portion of a protein which transduces the effector function signal and directs the cell to perform a specialized function.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term a “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, B7-H3, and a ligand that specifically binds with CD83, and the like. The intracellular signaling domain can comprise the entire intracellular portion, or the entire native intracellular signaling domain, of the molecule from which it is derived, or a functional fragment or derivative thereof.

The terms “immune effector cell” and “effector cell” are used interchangeably here. They refer to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

The term “immunity” and “immune response” are used herein interchangeably. As applied to a subject, it refers to the ability of the subject to elicit an immune response via his/her immune cells against an antigen, including without limitation tumor antigen, viral antigen, bacterial antigen, or neoantigen. As applied to a cell, it refers to the ability of the cell to generate a cellular response directly or indirectly against an antigen, including without limitation tumor antigen, viral antigen, bacterial antigen, or neoantigen.

The term “lymphoid cell” or “lymphoid cells” refers to any of the cells responsible for the production of immunity (or immune response) mediated by cells or antibodies and including lymphocytes, lymphoblasts, and plasma cells. Lymphoid cells include granulocytes such as asophils, eosinophils, and neutrophils; mast cells; monocytes which can develop into macrophages; antigen-presenting cells such as dendritic cells; and lymphocytes such as natural killer cells (NK cells), B cells, and T cells (including activated T cells). In some examples, T cells include both naive and memory cells (e.g. central memory or T_{CM}, effector memory or T_{EM} and effector memory RA or T_{EMRA}), effector cells (e.g. cytotoxic T cells or CTLs or Tc cells), helper cells (e.g. Th1, Th2, Th3, Th9, Th7, TFH), regulatory cells (e.g. Treg, and Tr1 cells), natural killer T cells (NKT cells), tumor infiltrating lymphocytes (TILs), lymphocyte-activated killer cells (LAKs), as T cells, γδ T cells, and similar unique classes of the T cell lineage.

The terms “tumor marker, “tumor antigen”, and “tumor-associated antigen” are used herein interchangeably, each referring to a molecule or fragment thereof expressed on the surface or inside of a cancer cell, or secreted or otherwise a molecule or fragment thereof derived from a cancer cell (e.g., circulating tumor DNA or circulating tumor RNA), and which is useful for the detecting a cancer cell or preferential targeting an agent to the cancer cell. A tumor antigen can be a marker expressed by both normal cells and cancer cells, e.g., a lineage marker, e.g., CD19 on B cells. A tumor antigen can be a cell surface molecule that is overexpressed or underexpressed in a cancer cell in comparison to a normal cell. A tumor antigen can also be a cell surface molecule that is inappropriately synthesized in the cancer cell, for instance, a molecule that contains deletions, additions or mutations in comparison to the molecule expressed on a normal cell. A tumor antigen can be expressed exclusively on the cell surface of a cancer cell, entirely or as a fragment (e.g., MHC/peptide), and not synthesized or expressed on the surface of a normal cell. A tumor antigen includes neoantigens encoded by tumor-specific mutated genes.

The term “transiently downregulated” as used herein generally means that a downregulation of expression or activity of a target molecule (e.g., PTPN2) is not permanent. A transient downregulation may not be a permanent downregulation. In some cases, a transient downregulation may involve downregulating (e.g., reducing) expression or activity of a target molecule for a period of time, followed by regaining at least a portion of expression or activity level of the target molecule that was previously downregulated. A transient downregulation can involve an intermittent downregulation of a target molecule (e.g., PTPN2).

The term “intermittent” is used herein to describe a process that is not continuous. An intermittent process may be followed by a break or stop. A plurality of intermittent processes may involve alternatively starting and stopping a same process or different processes. In some embodiments, the term “intermittent dosing regimen” as used here refers to a dosing regimen that comprises administering a pharmaceutical composition, followed by a rest period.

The term “side effect” as used herein refers to any complication, unwanted, or pathological outcome of a therapy (e.g., a cell therapy, an immunotherapy, etc.) that occurs in addition to or in place of a desired treatment outcome of the therapy. Examples of a side effect may include, but are not limited to, (i) off-target cell toxicity, (ii) on-target off-tumor toxicity, and/or (iii) autoimmunity (e.g., chronic autoimmunity). In an example, a side effect of a cell therapy involving a T-cell receptor fusion protein (TFP) and/or a chimeric antigen receptor (CAR) may include a graft-versus-host disease. In another example, a side effect of a cell therapy involving a TFP and/or a CAR may include death of a cell configured to express the TFP and/or the CAR.

Other examples of a side effect of a cell therapy may include, but are not limited to, disorders mediated by phagocytic cells, which includes macrophages and neutrophil granulocytes (Polymorphonuclear leukocytes, PMNs) and/or T cells. Examples include inflammatory skin diseases including psoriasis; responses associated with inflammatory bowel disease (such as Crohn's disease and ulcerative colitis); adult respiratory distress syndrome; dermatitis; CNS inflammatory disorders such as multiple sclerosis; uveitic disorders; allergic conditions such as eczema and asthma and other conditions involving infiltration of T cells and chronic inflammatory responses; skin hypersensitivity reactions (including poison ivy and poison oak); autoimmune diseases such as rheumatoid arthritis, systemic lupus erythematosus (SLE), diabetes mellitus, multiple sclerosis, Raynaud's syndrome, autoimmune thyroiditis, Sjogren's syndrome, juvenile onset diabetes, and immune responses associated with delayed hypersensitivity mediated by cytokines and T-lymphocytes typically found in tuberculosis, sarcoidosis, polymyositis, granulomatosis and vasculitis; pernicious anemia; multiple organ injury syndrome secondary to septicaemia or trauma; autoimmune haemolytic anemia; myethemia gravis; antigen-antibody complex mediated diseases; and/or all types of transplantation rejection, including graft vs. host or host vs. graft disease.

The term “efficacy” of a treatment or method, as used herein, can be measured based on changes in the course of disease or condition in response to such treatment or method. For example, the efficacy of a treatment or method of the present disclosure may be measured by its impact on signs or symptoms of a disease or condition of a subject, e.g., a tumor or cancer of the subject. A response may be achieved when a subject having the disease or condition experiences partial or total alleviation of the disease or condition, or reduction of one or more symptoms of the disease or condition. In an example, a response is achieved when a subject suffering from a tumor exhibits a reduction in the tumor size after the treatment or method, as provided in the present disclosure. In some examples, the efficacy may be measured by assessing cancer cell death, reduction of tumor (e.g., as evidenced by tumor size reduction), and/or inhibition of tumor growth, progression, and dissemination.

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

Unless defined otherwise, all technical and scientific terms used herein have the same meaning as is commonly understood to which the claimed subject matter belongs. In the event that there are a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

Definition of standard chemistry terms may be found in reference works, including but not limited to, Carey and Sundberg “Advanced Organic Chemistry 4′ Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

An “alkyl” group refers to a straight or branched hydrocarbon chain radical consisting solely of carbon and hydrogen atoms, containing no unsaturation. In some embodiments, the “alkyl” group may have 1 to 6 carbon atoms (whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated). The alkyl group of the compounds described herein may be designated as “C₁-C₆alkyl” or similar designations. By way of example only, “C₁-C₆alkyl” indicates that there are one to six carbon atoms in the alkyl chain, i.e., the alkyl chain is selected from the group consisting of methyl, ethyl, n-propyl, iso-propyl, n-butyl, iso-butyl, sec-butyl, t-butyl, n-pentyl, iso-pentyl, neo-pentyl, and hexyl. Alkyl groups can be substituted or unsubstituted. Depending on the structure, an alkyl group can be a monoradical or a diradical (i.e., an alkylene group).

An “alkoxy” refers to a “—O-alkyl” group, where alkyl is as defined herein.

The term “alkenyl” refers to a straight or branched hydrocarbon chain radical group consisting solely of carbon and hydrogen atoms, containing at least one carbon-carbon double bond. Non-limiting examples of an alkenyl group include —CH═CH₂, —C(CH₃)═CH₂, —CH═CHCH₃, —CH═C(CH₃)₂ and —C(CH₃)═CHCH₃. In some embodiments, an alkenyl groups may have 2 to 6 carbons. Alkenyl groups can be substituted or unsubstituted. Depending on the structure, an alkenyl group can be a monoradical or a diradical (i.e., an alkenylene group).

The term “alkynyl” refers to a straight or branched hydrocarbon chain radical group consisting solely of carbon and hydrogen atoms, containing at least one carbon-carbon triple bond. Non-limiting examples of an alkynyl group include —C═CH, —C═CCH₃, —C═CCH₂CH₃ and —C═CCH₂CH₂CH₃. In some embodiments, an alkynyl group can have 2 to 6 carbons. Alkynyl groups can be substituted or unsubstituted. Depending on the structure, an alkynyl group can be a monoradical or a diradical (i.e., an alkynylene group).

“Amino” refers to an —NH₂ group.

The term “alkylamine” or “alkylamino” refers to the —N(alkyl)ₓH_{y} group, where alkyl is as defined herein and x and y are selected from the group x=1, y=1 and x=2, y=0. When x=2, the alkyl groups, taken together with the nitrogen to which they are attached, can optionally form a cyclic ring system. “Dialkylamino” refers to an —N(alkyl)₂ group, where alkyl is as defined herein.

The term “aromatic” refers to a planar ring having a delocalized π-electron system containing 4n+2 π electrons, where n is an integer. Aromatic rings can be formed from five, six, seven, eight, nine, or more than nine atoms. Aromatics can be optionally substituted. The term “aromatic” includes both aryl groups (e.g., phenyl, naphthalenyl) and heteroaryl groups (e.g., pyridinyl, quinolinyl).

As used herein, the term “aryl” refers to a monocyclic aromatic ring wherein each of the atoms forming the ring is a carbon atom (e.g., phenyl) or a polycyclic ring system (e.g., bicyclic or tricyclic) wherein 1) at least one ring is carbocyclic and aromatic, 2) a bond to the remainder of the compound is directly bonded to a carbocyclic aromatic ring of the aryl ring system, and 3) the carbocyclic aromatic ring of the aryl ring system of 2) is not bonded (e.g., fused), either directly or through one or more aromatic rings, to a heteroaryl ring in the polycyclic ring system. Aryl rings can be formed by five, six, seven, eight, nine, or more than nine carbon atoms. Aryl groups can be optionally substituted. Examples of aryl groups include, but are not limited to phenyl, and naphthalenyl. Depending on the structure, an aryl group can be a monoradical or a diradical (i.e., an arylene group). As used herein, the aryl radical is a monocyclic, bicyclic, or tricyclic ring system. In embodiments, an aryl is a monocyclic ring. In embodiments, an aryl is a fused ring polycyclic system. In embodiments, an aryl is a bridged ring polycyclic system. In some embodiments the aryl is a “fused ring aryl” wherein the aryl ring is fused with a cycloalkyl or a heterocycloalkyl ring.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to,

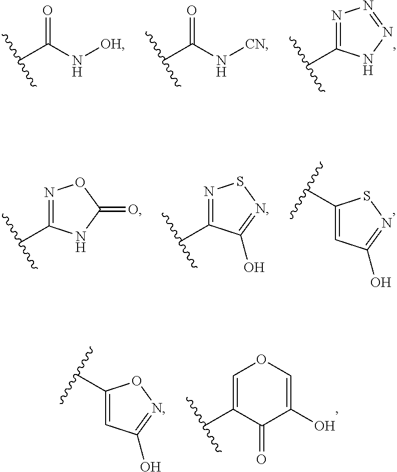

and the like.

The term “cycloalkyl” refers to a monocyclic carbocyclic saturated or partially unsaturated non-aromatic ring or a polycyclic carbocyclic (i.e., does not include heteroatom(s)) ring system (e.g., bicyclic or tricyclic) wherein 1) at least one ring is carbocyclic saturated or partially unsaturated and non-aromatic, 2) a bond to the remainder of the compound is directly bonded to a carbocyclic saturated or partially unsaturated non-aromatic ring of the ring system, and 3) the carbocyclic saturated or partially unsaturated non-aromatic ring of the ring system of 2) is not bonded (e.g., fused or spirocyclic), either directly or through one or more saturated or partially unsaturated and non-aromatic rings, to a heterocycloalkyl ring in the polycyclic ring system. Cycloalkyls may be saturated or partially unsaturated. In some embodiments, a cycloalkyl ring is a spirocyclic cycloalkyl ring. In embodiments, a cycloalkyl is a monocyclic ring. In embodiments, a cycloalkyl is a fused ring polycyclic system. In embodiments, a cycloalkyl is a bridged ring polycyclic system. In embodiments, a cycloalkyl is a spirocyclic polycyclic ring system. In some embodiments, cycloalkyl groups include groups having from 3 to 10 ring atoms. Depending on the structure, a cycloalkyl group can be a monoradical or a diradical (i.e., a cycloalkylene group).

The terms “heteroaryl” or, alternatively, “heteroaromatic” refers to an monocyclic aryl group that includes one or more ring heteroatoms selected from nitrogen, oxygen and sulfur; or a polycyclic ring system (e.g., bicyclic or tricyclic) wherein 1) at least one ring is aromatic and includes one or more heteroatoms selected from nitrogen, oxygen and sulfur and 2) a bond to the remainder of the compound is directly bonded to an aromatic ring including one or more heteroatoms selected from nitrogen, oxygen and sulfur or an aromatic ring bonded (e.g., fused), either directly or through one or more aromatic rings, to an aromatic ring including one or more heteroatoms selected from nitrogen, oxygen and sulfur, of the aryl ring system. As used herein, the heteroaryl radical may be a monocyclic, bicyclic, or tricyclic ring system, wherein at least one of the rings in the ring system is fully unsaturated (i.e., aromatic) and includes a heteroatom. In embodiments, a heteroaryl is a monocyclic ring. In embodiments, a heteroaryl is a fused ring polycyclic system. In embodiments, a heteroaryl is a bridged ring polycyclic system. In some embodiments is a “fused ring heteroaryl” wherein the heteroaryl ring is fused with a cycloalkyl, aryl, or heterocycloalkyl ring. An N-containing “heteroaromatic” or “heteroaryl” moiety refers to an aromatic group in which at least one of the skeletal atoms of the ring is a nitrogen atom. Depending on the structure, a heteroaryl group can be a monoradical or a diradical (i.e., a heteroarylene group).

A “heterocycloalkyl” group or “heteroalicyclic” group or “heterocyclyl” group refers to a cycloalkyl group, wherein at least one skeletal ring atom of a saturated or partially unsaturated non-aromatic ring is a heteroatom selected from nitrogen, oxygen, phosphorus, and sulfur. In embodiments, the nitrogen, phosphorus, or sulfur atoms may optionally be oxidized and the nitrogen heteroatom may optionally be quaternized. A heterocycloalkyl refers to a monocyclic saturated or partially unsaturated non-aromatic ring including one or more heteroatoms or a polycyclic ring system (e.g., bicyclic or tricyclic) wherein 1) at least one ring is saturated or partially unsaturated, non-aromatic, and includes one or more heteroatoms and 2) a bond to the remainder of the compound is directly bonded to a ring of the ring system that is a saturated or partially unsaturated and non-aromatic ring that includes one or more heteroatoms or a non-aromatic ring bonded (e.g., fused), either directly or through one or more saturated or partially unsaturated and non-aromatic rings, to a saturated or partially unsaturated and non-aromatic ring that includes one or more heteroatoms of the ring system. Heterocycloalkyls may be saturated or partially unsaturated. The term heterocycloalkyl also includes all ring forms of the carbohydrates, including but not limited to the monosaccharides, the disaccharides and the oligosaccharides. In some embodiments, a heterocycloalkyl ring is a spirocyclic heterocycloalkyl ring. In embodiments, a heterocycloalkyl is a monocyclic ring. In embodiments, a heterocycloalkyl is a fused ring polycyclic system. In embodiments, a heterocycloalkyl is a bridged ring polycyclic system. In embodiments, a heterocycloalkyl is a spirocyclic polycyclic ring system. Unless otherwise noted, heterocycloalkyls have from 2 to 13 carbons in the ring or ring system. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Depending on the structure, a heterocycloalkyl group can be a monoradical or a diradical (i.e., a heterocycloalkylene group).

The term “halo” or, alternatively, “halogen” means fluoro, chloro, bromo and iodo.

The term “haloalkyl” refers to an alkyl group that is substituted with one or more halogens. The halogens may the same or they may be different. Non-limiting examples of haloalkyls include —CH₂C₁, —CF₃, —CHF₂, —CH₂CF₃, —CF₂CF₃, and the like.

The terms “fluoroalkyl” and “fluoroalkoxy” include alkyl and alkoxy groups, respectively, that are substituted with one or more fluorine atoms. Non-limiting examples of fluoroalkyls include —CF₃, —CHF₂, —CH₂F, —CH₂CF₃, —CF₂CF₃, —CF₂CF₂CF₃, —CF(CH₃)₃, and the like. Non-limiting examples of fluoroalkoxy groups, include —OCF₃, —OCHF₂, —OCH₂F, —OCH₂CF₃, —OCF₂CF₃, —OCF₂CF₂CF₃, —OCF(CH₃)₂, and the like.

The term “heteroalkyl” refers to an alkyl radical where one or more skeletal chain atoms is selected from an atom other than carbon, e.g., oxygen, nitrogen, sulfur, phosphorus, silicon, or combinations thereof. The heteroatom(s) may be placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH₂—NH—OCH₃, —CH₂—O—Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃. Excluding the number of heteroatoms, a “heteroalkyl” may have from 1 to 6 carbon atoms.

The term “oxo” refers to the ═O radical.

The term “bond” or “single bond” refers to a chemical bond between two atoms, or two moieties when the atoms joined by the bond are considered to be part of larger substructure.

The term “moiety” refers to a specific segment or functional group of a molecule. Chemical moieties are often recognized chemical entities embedded in or appended to a molecule.

As used herein, the substituent “R” appearing by itself and without a number designation refers to a substituent selected from among from alkyl, haloalkyl, heteroalkyl, alkenyl, cycloalkyl, aryl, heteroaryl (bonded through a ring carbon), and heterocycloalkyl.

“Optional” or “optionally” means that a subsequently described event or circumstance may or may not occur and that the description includes instances when the event or circumstance occurs and instances in which it does not.

The term “optionally substituted” or “substituted” means, unless otherwise specified, that the referenced group may be substituted with one or more additional group(s) individually and independently selected from alkyl, alkenyl, alkynyl, cycloalkyl, aryl, heteroaryl, heterocycloalkyl, —OH, oxo, alkoxy, aryloxy, alkylthio, arylthio, alkylsulfoxide, arylsulfoxide, alkylsulfone, arylsulfone, —CN, C₁-C₆alkylalkyne, halo, acyl, acyloxy, —CO₂H, —CO₂-alkyl, nitro, haloalkyl, fluoroalkyl, and amino, including mono- and di-substituted amino groups (e.g. —NH₂, —NHR, —N(R)₂), and the protected derivatives thereof. By way of example, an optional substituents may be L^{s}R^{s}, wherein each L³ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, —(C₁-C₆alkyl)-, or —(C₂-C₆alkenyl)-; and each R³ is independently selected from among H, (C₁-C₆alkyl), (C₃-C₈cycloalkyl), aryl, heteroaryl, heterocycloalkyl, and C₁-C₆heteroalkyl. The protecting groups that may form the protective derivatives of the above substituents are found in sources such as Greene and Wuts, above.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula -R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O-Rc-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

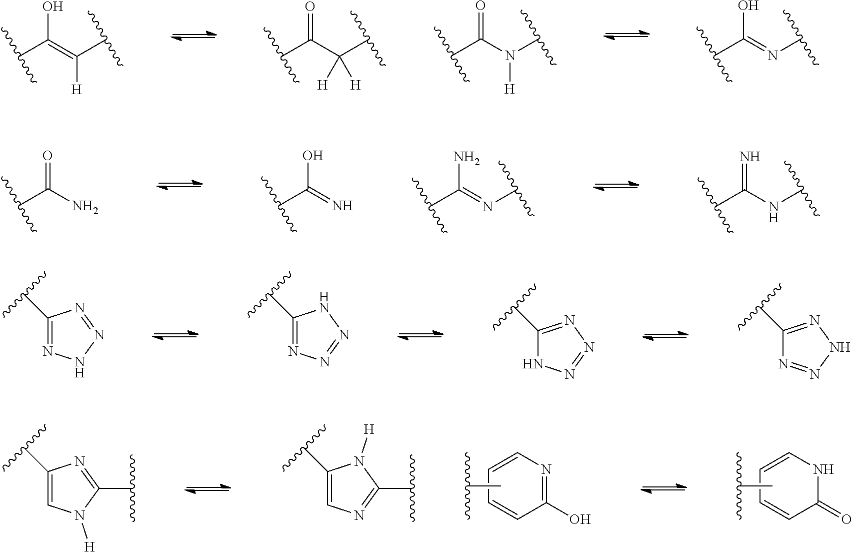

In some instances, the compounds disclosed herein exist in tautomeric forms. The structures of said compounds are illustrated in the one tautomeric form for clarity. The alternative tautomeric forms are expressly included in this disclosure, such as, for example, the structures illustrated below.

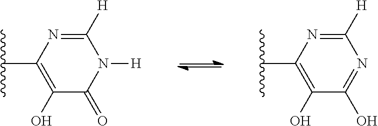 [SMILES: *c1nc[nH]c(=O)c1O ; *c1ncnc(O)c1O]

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

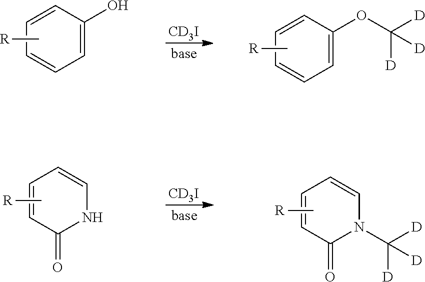

Deuterium-transfer reagents, such as lithium aluminum deuteride (LiAID₄), are employed to transfer deuterium under reducing conditions to the reaction substrate. The use of LiAID₄ is illustrated, by way of example only, in the reaction schemes below.

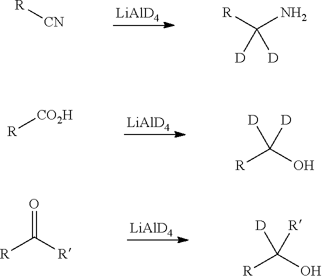

Deuterium gas and palladium catalyst are employed to reduce unsaturated carbon-carbon linkages and to perform a reductive substitution of aryl carbon-halogen bonds as illustrated, by way of example only, in the reaction schemes below.

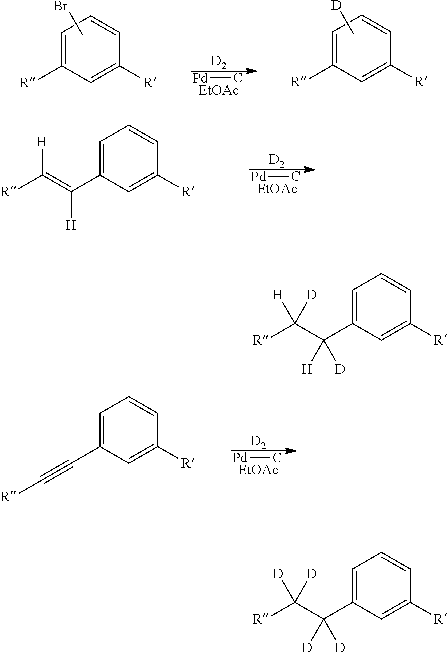

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Prodrug” as used herein is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. Typically, prophylactic benefit includes reducing the incidence and/or worsening of one or more diseases, conditions, or symptoms under treatment (e.g. as between treated and untreated populations, or between treated and untreated states of a subject).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

An “antigen” is a moiety or molecule that contains an epitope, and, as such, also specifically binds to an antibody.

An “antigen binding unit” may be whole or a fragment (or fragments) of a full-length antibody, a structural variant thereof, a functional variant thereof, or a combination thereof. A full-length antibody may be, for example, a monoclonal, recombinant, chimeric, deimmunized, humanized and human antibody. Examples of a fragment of a full-length antibody may include, but are not limited to, variable heavy (VH), variable light (VL), a heavy chain found in camelids, such as camels, llamas, and alpacas (VHH or V_{H}H), a heavy chain found in sharks (V-NAR domain), a single domain antibody (sdAb, i.e., “nanobody”) that comprises a single antigen-binding domain, Fv, Fd, Fab, Fab′, F(ab′)₂, and “r IgG” (or half antibody). Examples of modified fragments of antibodies may include, but are not limited to scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, minibodies (e.g., (VH-VL-CH₃)₂, (scFv-CH₃)₂, ((scFv)₂-CH₃+CH₃), ((scFv)₂-CH₃) or (scFv-CH₃-scFv)₂), and multibodies (e.g., triabodies or tetrabodies).

The term “antibody” and “antibodies” encompass any antigen binding units, including without limitation: monoclonal antibodies, human antibodies, humanized antibodies, camelised antibodies, chimeric antibodies, and any other epitope-binding fragments.

The term “in vivo” refers to an event that takes place in a subject's body.

The term “ex vivo” refers to an event that first takes place outside of the subject's body for a subsequent in vivo application into a subject's body. For example, an ex vivo preparation may involve preparation of cells outside of a subject's body for the purpose of introduction of the prepared cells into the same or a different subject's body.

The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

Described herein below are compounds, including compounds having PTPN2 inhibitor activity (i.e., PTPN2 inhibitors). In an aspect is provided a compound of Formula (I), or a pharmaceutically acceptable salt or solvate thereof:

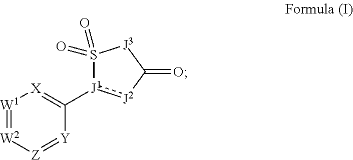

wherein:
either: W¹ is C(R¹), and W² is C(R^{2a}) or N; or W¹ is C(R^{1a}) or N, and W² is C(R²);
R¹ is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are substituted with one or more substituents independently selected from R⁶ and R^{6′}, wherein ring heteroatoms of the 5 to 12 membered heterocycloalkyl and 5 to 12 membered heteroaryl are independently selected from N, O, and S;
R^{1a} is selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20a};
R² is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are substituted with one or more substituents independently selected from R⁷ and R^{7′}, wherein ring heteroatoms of the 5 to 12 membered heterocycloalkyl and 5 to 12 membered heteroaryl are independently selected from N, O, and S;
R^{2a} are independently selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20b};
X is N or C(R³);
Y is N or C(R⁴);
Z is N or C(R⁵);
R³ is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R⁴ is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20e};
each R⁶ and R⁷ are independently selected from halogen, oxo, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁶;
each R^{6′} and R^{7′} are independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀ aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶;
J¹ is N, C, or C(R¹);
J² is N, N(R⁹), C(R⁹), C(R⁹)(R^{9a}), or C(O);
J³ is N(R¹⁰) or C(R¹⁰)(R^{10a});
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉ heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉ heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20l};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted with one, two, or three R^{20m};
each R¹⁶ is independently selected from —C₁₋₆alkylene-OP(O)(OR^{16a})(OR¹⁶ⁱ*) and —P(O)(OR^{16a})(OR⁶Ib); wherein the C₁₋₆alkylene is optionally substituted with one, two, or three R²⁰ⁿ; or —C₁₋₆alkylene-OC(O)—R^{16c} wherein C₁₋₆alkylene is optionally substituted with one, two, or three R²⁰ⁿ;
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20o};
R^{16c} is halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —CH₂—C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three of R^{20o};
each R^{20a}, R^{20b}, R^{20c}, R^{20d}, R^{20e}, R^{20f}, R^{20h}, R²⁰ⁱ, R^{20j}, R^{20k}, R^{20l}, R^{20m}, R²⁰ⁿ, and R^{20o} are each independently selected from oxo, —CN, halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl optionally substituted with one or more R²⁶, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉ heteroaryl;
each R²⁶ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁷;
each R²⁷ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f};
[symbol] indicates a single or double bond such that all valences are satisfied.

In embodiments, a compound described herein, or a pharmaceutically acceptable salt or solvate thereof, has the structure of Formula (Ia):

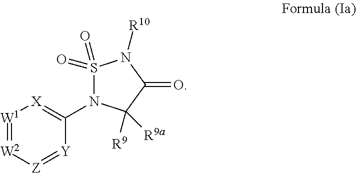

In embodiments, a compound described herein, or a pharmaceutically acceptable salt or solvate thereof, has the structure of Formula (Ib):

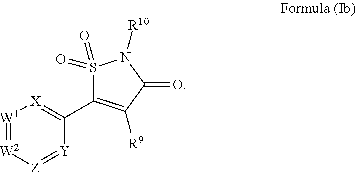

In embodiments, a compound described herein, or a pharmaceutically acceptable salt or solvate thereof, has the structure of Formula (Ic):

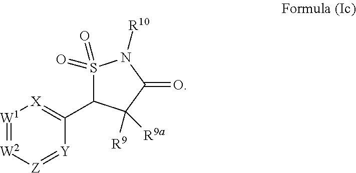

In embodiments, a compound described herein, or a pharmaceutically acceptable salt or solvate thereof, has the formula:

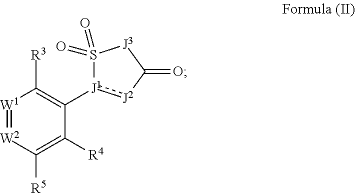

In embodiments, a compound described herein, or a pharmaceutically acceptable salt or solvate thereof, has the structure of Formula (IIa):

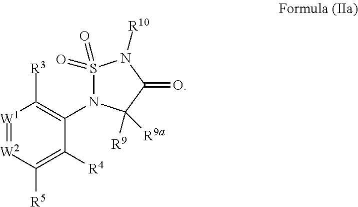

In embodiments, a compound described herein, or a pharmaceutically acceptable salt or solvate thereof, has the structure of Formula (IIb):

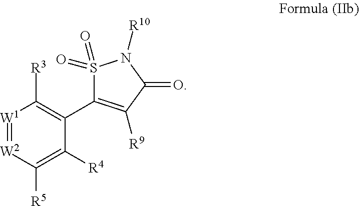

In embodiments, a compound described herein, or a pharmaceutically acceptable salt or solvate thereof, has the structure of Formula (IIc):

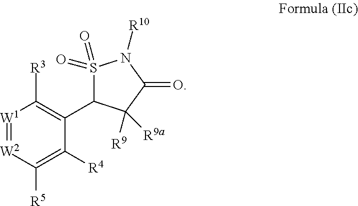

In embodiments, the compound of Formula (IIa) has the structure of Formula (IId):

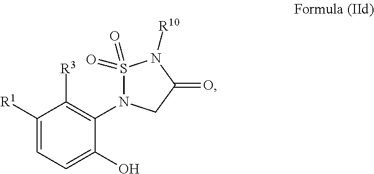

such as Formula (IId-1)

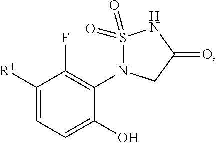

or a pharmaceutically acceptable salt or solvate thereof,
In an aspect is provided a compound of Formula (IId-1), or a pharmaceutically acceptable salt or solvate thereof:

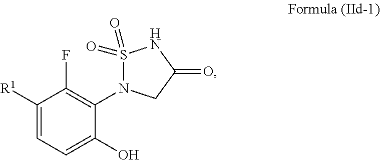

wherein:
R¹ is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are substituted with one or more substituents independently selected from R⁶ and R^{6′}, wherein ring heteroatoms of the 5 to 12 membered heterocycloalkyl and 5 to 12 membered heteroaryl are independently selected from N, O, and S;
each R⁶ is independently selected from halogen, oxo, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —C(NR¹⁴)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R²⁶;
each R^{6′} is independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —C(O)OR¹², —OC(O)N(R¹²)(R¹³), —C(NR¹⁴)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶;
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉ heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, —CN, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20l};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted with one, two, or three R^{20m};
each R^{20f}, R^{20k}, R^{20l}, and R^{20m} is independently selected from oxo, —CN, halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl optionally substituted with one or more R²⁶, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is independently selected from C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl;
each R²⁶ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁷; and
each R²⁷ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20f}.

In embodiments, for a compound of Formula (II-d1):
R¹ is selected from C₅₋₁₀cycloalkyl, 5 to 10 membered heterocycloalkyl, C₆aryl, and 5 to 10 membered heteroaryl, wherein C₅₋₁₀cycloalkyl, 5 to 10 membered heterocycloalkyl, C₆aryl, and 5 to 10 membered heteroaryl are substituted with one or more R⁶, wherein ring heteroatoms of the 5 to 10 membered heterocycloalkyl and 5 to 10 membered heteroaryl are independently selected from N, O, and S; and
each R⁶ is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀ aryl, —OR¹², —N(R¹²)(R¹³), —C(O)OR¹², —C(NR¹⁴)N(R¹²)(R¹³), —N(R¹⁴)S(O)₂R¹⁵, —C(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₁₀cycloalkyl, C₂₋₉heterocycloalkyl, and C₆₋₁₀aryl are optionally substituted with one, two, or three R²⁶.

In embodiments, for a compound of Formula (II-d1), R¹ is selected from pyrrolyl, pyrazolyl, imidazolyl, triazolyl, pyrrolinyl, pyridinyl, tetrahydropyridinyl, piperidinyl, and piperazinyl, each of which is optionally substituted with one or more R⁶. In embodiments, R¹ is selected from pyrrolyl, pyrazolyl, imidazolyl, triazolyl, pyrrolinyl, pyridinyl, tetrahydropyridinyl, piperidinyl, and piperazinyl, each of which is optionally substituted with one or more substituents independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, —OR¹², —N(R¹²)(R¹³), —C(O)OR¹², —C(NR¹⁴)N(R¹²)(R¹³), —N(R¹⁴)S(O)₂R¹⁵, —C(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₁₀cycloalkyl, C₂₋₉heterocycloalkyl, and C₆₋₁₀aryl are optionally substituted with one, two, or three R²⁶. In some embodiments, each R¹³ is independently selected from hydrogen, —CN, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20l}. In some embodiments, R¹ is —C(NR¹⁴)N(R¹²)(R¹³), such as —C(NH)NH₂ or —C(NH)NHCN.

In embodiments, for a compound of Formula (II-d1), R¹ is selected from:

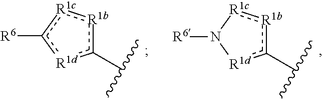

wherein R^{1c} is not N or N(R^{6c″}) when R^{1b} is C(R^{6b}) and R^{1d} is C(R^{6d});

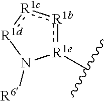

wherein R¹ is not a substituted pyrrolyl; and

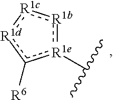

wherein R¹ is not a substituted imidazolyl wherein R^{1d} and R^{1e} are N;
R^{1b} is N, N(R^{6b″}), C(R^{6b}), or C(R^{6b})(R^{6b′});
R^{1c} is N, N(R^{6c″}), C(R^{6c}), or C(R^{6c})(R^{6c′});
R^{1d} is N, N(R^{6d″}), C(R^{6d}), or C(R^{6d})(R^{6d′});
R^{1e} is N, C, or C(R^{6e});
R^{6b}, R^{6b′}, R^{6c}, R^{6c′}, R^{6d}, R^{6d′}, and R^{6e} are independently selected from hydrogen, halogen, —CN, C₁₋₆alkyl, C₂₋₆alkenyl, C₂₋₆alkynyl, C_{3- 10}cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20a};
R^{6b″}, R^{6c″}, and R^{6d″} are independently selected from hydrogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
each R⁶ is independently selected from halogen, oxo, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁶; and
each R^{6′} is independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In some embodiments, for a compound of Formula (II-d1), R¹ is selected from

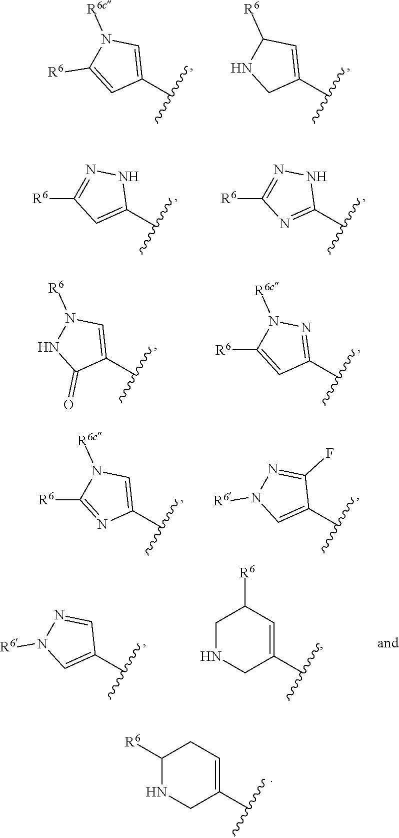

In some embodiments, R¹ is selected from

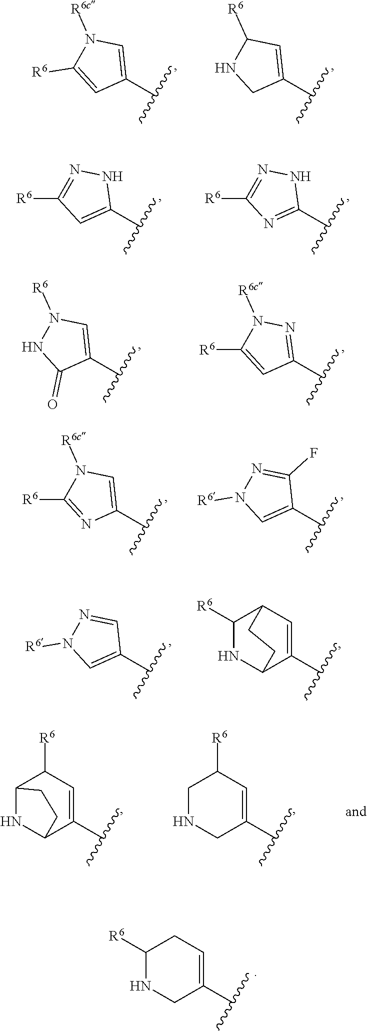

In some embodiments, R¹ is selected from

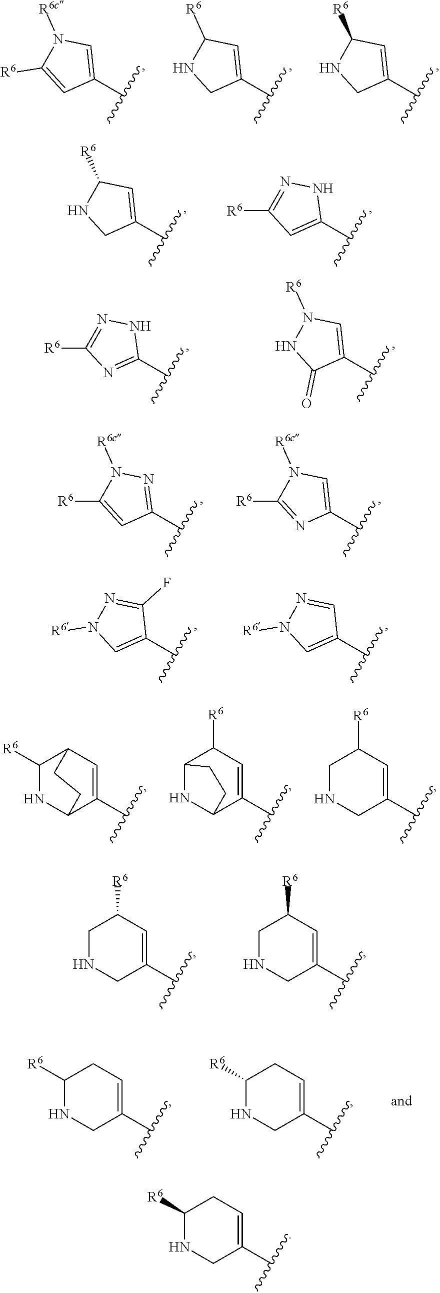

In some embodiments, R¹ is selected from

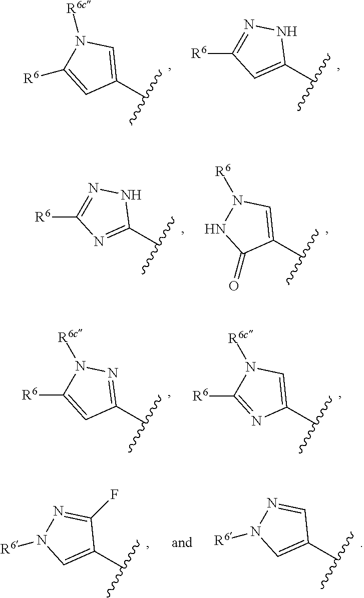

In some embodiments, R¹ is selected from

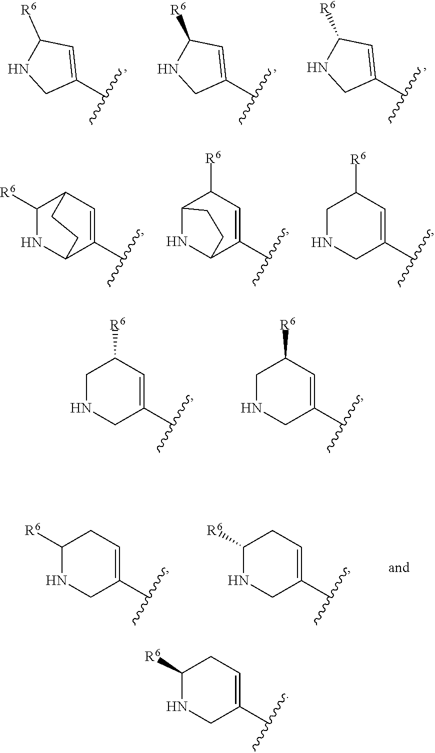

In some embodiments, for a compound of Formula (II-d1), each R^{20f}, R^{20k}, R^{20l}, and R^{20m} is independently selected from oxo, —CN, halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In an aspect is provided a compound of Formula (III), or a pharmaceutically acceptable salt or solvate thereof:

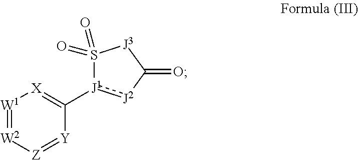

wherein:
either:
W¹ is C(R¹), and W² is C(R^{2a}) or N; or
W¹ is C(R^{1a}) or N, and W² is C(R²);
R¹ is selected from C₁₋₆alkyl, C₂₋₆alkenyl, C₂₋₆alkynyl, C₅₋₁₂cycloalkyl, 5 to 12 membered heterocycloalkyl, C₆₋₁₂aryl, and 5 to 12 membered heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₅₋₁₂cycloalkyl, 5 to 12 membered heterocycloalkyl, C₆₋₁₂aryl, and 5 to 12 membered heteroaryl are optionally substituted with one or more substituents independently selected from R⁶ and R^{6′};
R^{1a} is selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20a};
R² is selected from C₁₋₆alkyl, C₂₋₆alkenyl, C₂₋₆alkynyl, C₅₋₁₂cycloalkyl, 5 to 12 membered heterocycloalkyl, C₆₋₁₂aryl, and 5 to 12 membered heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₅₋₁₂cycloalkyl, 5 to 12 membered heterocycloalkyl, C₆₋₁₂aryl, and 5 to 12 membered heteroaryl are optionally substituted with one or more substituents independently selected from R⁷ and R^{7′};
R^{2a} are independently selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20b};
X is N or C(R³);
Y is N or C(R⁴).
Z is N or C(R⁵);
at least one of W¹, W², X, Y, and Z is N;
R³ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20c};
R⁴ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20d};
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20e};
each R⁶ and R⁷ are independently selected from halogen, oxo, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁶;
each R^{6′} and R^{7′} are independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀ aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶;
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉ heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉ heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20l};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted with one, two, or three R^{20m};
each R¹⁶ is independently selected from —C₁₋₆alkylene-OP(O)(OR^{16a})(OR^{6b}) and —P(O)(OR^{16a})(OR^{6b}); wherein the C₁₋₆alkylene is optionally substituted with one, two, or three R²ⁿ;
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20o};
each R^{20a}, R^{20b}, R^{20c}, R^{20d}, R^{20e}, R^{20f}, R^{20h}, R²⁰ⁱ, R^{20j}, R^{20k}, R^{20l}, R^{20m}, R²⁰ⁿ, and R^{20o} are each independently selected from oxo, —CN, halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl optionally substituted with one or more R²⁶, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl;
each R²⁶ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁷;
each R²⁷ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20f};
[symbol] indicates a single or double bond such that all valences are satisfied.

In some embodiments, X is C(R³). In embodiments, Y is C(R⁴). In additional embodiments, Z is C(R⁵). In some embodiments, X is N. In additional embodiments, Y is N. In embodiments, Z is N. In some embodiments, W¹ is C(R¹). In additional embodiments, W¹ is C(R^{1a}). In some embodiments, W² is C(R^{2a}). In embodiments, W² is C(R²). In additional embodiments, W¹ is N. In some embodiments, W² is N. In embodiments, J² is CH₂. In some embodiments, J² is C(H). In additional embodiments, J³ is N(H). In embodiments, J³ is C(R¹⁰)(R^{10a}). In some embodiments, J³ is CH(R¹⁰). In some embodiments, R¹⁰ is hydrogen.

In embodiments, R⁴ is selected from halogen, —OR¹², and C₁₋₆alkyl optionally substituted with one, two, or three R^{20d}. In some embodiments, R⁴ is —OH.

In embodiments, R⁵ is selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20e}. In embodiments, R⁵ is selected from hydrogen, halogen, and C₁₋₆alkyl optionally substituted with one, two, or three R^{20e}. In some embodiments, R⁵ is hydrogen. In some embodiments, R⁵ is —OH. In some embodiments, R⁵ is halogen. In some embodiments, R⁵ is —F.

In additional embodiments, R³ is selected from halogen and C₁₋₆alkyl optionally substituted with one, two, or three R^{20c}. In embodiments, R³ is halogen. In embodiments, R³ is F. In some embodiments, R³ is C₁.

In embodiments,
R¹ is selected from C₅₋₁₂cycloalkyl, 5 to 12 membered heterocycloalkyl, C₇₋₁₂aryl, and 5 to 12 membered heteroaryl, wherein C₅₋₁₂ cycloalkyl, 5 to 12 membered heterocycloalkyl, C₇₋₁₂aryl, and 5 to 12 membered heteroaryl are optionally substituted with one or more substituents independently selected from R⁶ and R^{6′}, wherein ring heteroatoms of the 5 to 12 membered heterocycloalkyl and 5 to 12 membered heteroaryl are independently selected from C, N, O, and S; and
R² is selected from C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl, wherein C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl are optionally substituted with one or more substituents independently selected from R⁷ and R^{7′}, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S.

In embodiments,
R¹ is selected from C₅₋₁₂cycloalkyl, 5 to 12 membered heterocycloalkyl, C₇₋₁₂aryl, and 5 to 12 membered heteroaryl, wherein C₅₋₁₂ cycloalkyl, 5 to 12 membered heterocycloalkyl, C₇₋₁₂aryl, and 5 to 12 membered heteroaryl are substituted with one or more substituents independently selected from R⁶ and R^{6′}, wherein ring heteroatoms of the 5 to 12 membered heterocycloalkyl and 5 to 12 membered heteroaryl are independently selected from C, N, O, and S; and
R² is selected from C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl, wherein C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl are substituted with one or more substituents independently selected from R⁷ and R^{7′}, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S.

In embodiments,
R¹ is selected from C₅₋₁₂cycloalkyl, 5 to 12 membered heterocycloalkyl, C₇₋₁₂aryl, and 5 to 12 membered heteroaryl, wherein C₅₋₁₂ cycloalkyl, 5 to 12 membered heterocycloalkyl, C₇₋₁₂aryl, and 5 to 12 membered heteroaryl are substituted with one or more substituents independently selected from R⁶ and R^{6′}, wherein ring heteroatoms of the 5 to 12 membered heterocycloalkyl and 5 to 12 membered heteroaryl are independently selected from C, N, O, and S; and
R² is selected from C₅₋₁₂cycloalkyl, 5 to 12 membered partially unsaturated heterocycloalkyl, 6 to 12 membered saturated heterocycloalkyl, and 5 to 12 membered heteroaryl, wherein C₅₋₁₂cycloalkyl, 5 to 12 membered partially unsaturated heterocycloalkyl, 6 to 12 membered saturated heterocycloalkyl, and 5 to 12 membered heteroaryl are substituted with one or more substituents independently selected from R⁷ and R^{7′}, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S.

In embodiments, R¹ is selected from C₅₋₁₂cycloalkyl, 5 to 12 membered heterocycloalkyl, C₇₋₁₂aryl, and 5 to 12 membered heteroaryl, wherein C₅₋₁₂cycloalkyl, 5 to 12 membered heterocycloalkyl, C₇₋₁₂aryl, and 5 to 12 membered heteroaryl are substituted with one or more substituents independently selected from R⁶ and R^{6′}, wherein ring heteroatoms of the 5 to 12 membered heterocycloalkyl and 5 to 12 membered heteroaryl are independently selected from C, N, O, and S.

In embodiments, R² is selected from C₅₋₁₂cycloalkyl, 5 to 12 membered partially unsaturated heterocycloalkyl, 6 to 12 membered saturated heterocycloalkyl, and 5 to 12 membered heteroaryl, wherein C₅₋₁₂cycloalkyl, 5 to 12 membered partially unsaturated heterocycloalkyl, 6 to 12 membered saturated heterocycloalkyl, and 5 to 12 membered heteroaryl are substituted with one or more substituents independently selected from R⁷ and R^{7′}, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S. In embodiments, R² is not phenyl. In embodiments, R² is not phenyl substituted with one or more substituents independently selected from R⁷ and R^{7′}. In embodiments, R² is not phenyl substituted with methoxy. In embodiments, R² is not phenyl substituted with 3-methoxy.

In embodiments,
R¹ is selected from triazolyl, pyrazolyl, pyrrolyl, imidazolyl, phenyl, pyridyl, pyrimidinyl, pyridazinyl, pyrazinyl, and triazinyl, wherein triazolyl, pyrazolyl, pyrrolyl, imidazolyl, phenyl, pyridyl, pyrimidinyl, pyridazinyl, pyrazinyl, and triazinyl are substituted with one or more substituents independently selected from R⁶ and R^{6′}; and
R² is selected from triazolyl, pyrazolyl, pyrrolyl, imidazolyl, phenyl, pyridyl, pyrimidinyl, pyridazinyl, pyrazinyl, and triazinyl, wherein triazolyl, pyrazolyl, pyrrolyl, imidazolyl, phenyl, pyridyl, pyrimidinyl, pyridazinyl, pyrazinyl, and triazinyl are substituted with one or more substituents independently selected from R⁷ and R^{7′}.

In embodiments, R¹ is triazolyl substituted with one or more substituents independently selected from R⁶. In embodiments, R¹ is pyrazolyl substituted with one or more substituents independently selected from R⁶. In embodiments, R¹ is pyrrolyl substituted with one or more substituents independently selected from R⁶. In embodiments, R¹ is imidazolyl substituted with one or more substituents independently selected from R⁶. In embodiments, R¹ is phenyl substituted with one or more substituents independently selected from R⁶. In embodiments, R¹ is pyridyl substituted with one or more substituents independently selected from R⁶. In embodiments, R¹ is pyrimidinyl substituted with one or more substituents independently selected from R⁶. In embodiments, R¹ is pyridazinyl substituted with one or more substituents independently selected from R⁶. In embodiments, R¹ is pyrazinyl substituted with one or more substituents independently selected from R⁶. In embodiments, R¹ is triazinyl substituted with one or more substituents independently selected from R⁶.

In embodiments, R² is triazolyl substituted with one or more substituents independently selected from R⁷. In embodiments, R² is pyrazolyl substituted with one or more substituents independently selected from R⁷. In embodiments, R² is pyrrolyl substituted with one or more substituents independently selected from R⁷. In embodiments, R² is imidazolyl substituted with one or more substituents independently selected from R⁷. In embodiments, R² is phenyl substituted with one or more substituents independently selected from R⁷. In embodiments, R² is pyridyl substituted with one or more substituents independently selected from R⁷. In embodiments, R² is pyrimidinyl substituted with one or more substituents independently selected from R⁷. In embodiments, R² is pyridazinyl substituted with one or more substituents independently selected from R⁷. In embodiments, R² is pyrazinyl substituted with one or more substituents independently selected from R⁷. In embodiments, R² is triazinyl substituted with one or more substituents independently selected from R⁷.

In embodiments, R¹ is triazolyl substituted with one or more substituents independently selected from R^{6′}. In embodiments, R¹ is pyrazolyl substituted with one or more substituents independently selected from R^{6′}. In embodiments, R¹ is pyrrolyl substituted with one or more substituents independently selected from R^{6′}. In embodiments, R¹ is imidazolyl substituted with one or more substituents independently selected from R^{6′}. In embodiments, R¹ is phenyl substituted with one or more substituents independently selected from R^{6′}. In embodiments, R¹ is pyridyl substituted with one or more substituents independently selected from R^{6′}. In embodiments, R¹ is pyrimidinyl substituted with one or more substituents independently selected from R^{6′}. In embodiments, R¹ is pyridazinyl substituted with one or more substituents independently selected from R^{6′}. In embodiments, R¹ is pyrazinyl substituted with one or more substituents independently selected from R^{6′}. In embodiments, R¹ is triazinyl substituted with one or more substituents independently selected from R^{6′}.

In embodiments, R² is triazolyl substituted with one or more substituents independently selected from R^{7′}. In embodiments, R² is pyrazolyl substituted with one or more substituents independently selected from R^{7′}. In embodiments, R² is pyrrolyl substituted with one or more substituents independently selected from R^{7′}. In embodiments, R² is imidazolyl substituted with one or more substituents independently selected from R^{7′}. In embodiments, R² is phenyl substituted with one or more substituents independently selected from R^{7′}. In embodiments, R² is pyridyl substituted with one or more substituents independently selected from R^{7′}. In embodiments, R² is pyrimidinyl substituted with one or more substituents independently selected from R^{7′}. In embodiments, R² is pyridazinyl substituted with one or more substituents independently selected from R^{7′}. In embodiments, R² is pyrazinyl substituted with one or more substituents independently selected from R^{7′}. In embodiments, R² is triazinyl substituted with one or more substituents independently selected from R^{7′}.

In embodiments, R¹ is

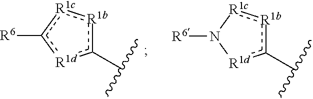

wherein R^{1c} is not N or N(R^{6c″}) when R^{1b} is C(R^{6b}) and R^{1d} is C(R^{6d});

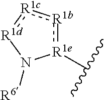

wherein R¹ is not a substituted pyrrolyl;

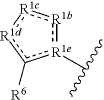

wherein R¹ is not a substituted imidazolyl wherein R^{1d} and R^{1e} are N; or fluoro-substituted pyrazolyl optionally further substituted with one, two, or three substituents independently selected from R⁶ and R^{6′}; R^{1b} is O, S, N, N(R^{6b″}), C(R^{6b″}), or C(R^{6b})(R^{6b′});
R^{1c} is O, S, N, N(R^{6c″}), C(R^{6c}), or C(R^{6c})(R^{6c′});
R^{1d} is O, S, N, N(R^{6d″}), C(R^{6d}), or C(R^{6d})(R^{6d′});
R^{1e} is N or C(R^{6e});
R^{6b}, R^{6b′}, R^{6c}, R^{6c′}, R^{6d}, R^{6d′}, and R^{6e} are independently selected from hydrogen, halogen, —CN, C₁₋₆alkyl, C₂₋₆alkenyl, C₂₋₆alkynyl, C_{3- 10}cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20a};
R^{6b″}, R^{6c″}, and R^{6d″} are independently selected from hydrogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R² is

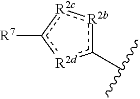

wherein R² is not a substituted imidazolyl wherein R^{2c} and R^{2d} are N;

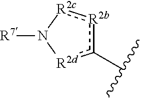

wherein R² is not a substituted pyrazolyl or pyrrolyl;

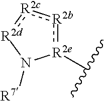

wherein R² is not a substituted pyrrolyl or imidazolyl;

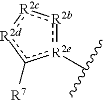

wherein R² is not a substituted imidazolyl wherein R^{2d} and R^{2e} are N, or R^{2b} and R^{2d} are N or NH; or Fluoro-substituted pyrazolyl optionally further substituted with one, two, or three substituents independently selected from R⁷ and R^{7′};
R^{2b} is O, S, N, N(R^{7b″}), C(R^{7b}), or C(R^{7b})(R^{7b′});
R^{2c} is O, S, N, N(R^{7c″}), C(R^{7c}), or C(R^{7c})(R^{c′});
R^{2d} is O, S, N, N(R^{7d″}), C(R^{7d}), or C(R^{7d})(R^{7d′});
R^{2e} is N or C(R^{7e});
R^{7b}, R^{7b′}, R^{7c}, R^{7c′}, R^{7d}, R^{7d′}, and R^{7e} are independently selected from hydrogen, halogen, —CN, C₁₋₆alkyl, C₂₋₆alkenyl, C₂₋₆alkynyl, C_{3- 10}cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20b};
R^{7b″}, R^{7c″}, and R^{7d″} are independently selected from hydrogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b}; and
each R⁶ and R⁷ are independently selected from halogen, oxo, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁶;
each R^{6′} and R^{7′} are independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀ aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In some embodiments, R¹ is

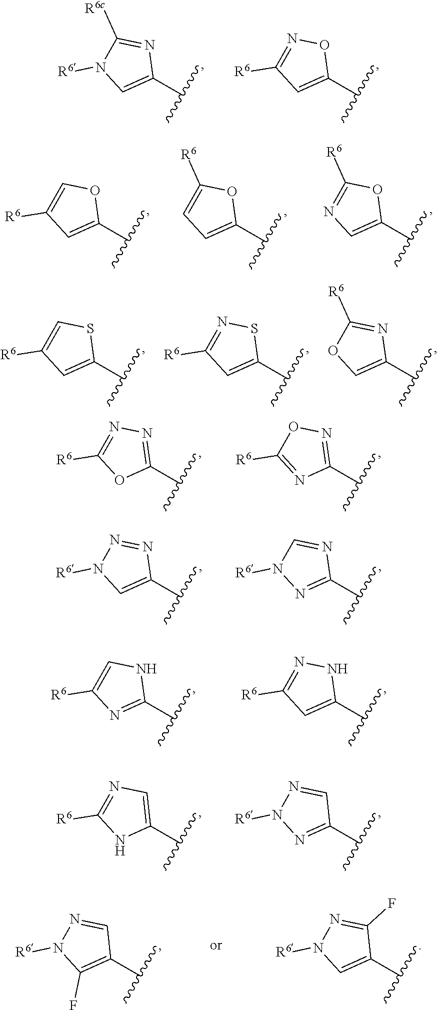

In some embodiments, R¹ is

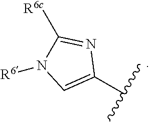

In some embodiments, R¹ is

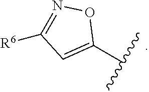

In some embodiments, R¹ is

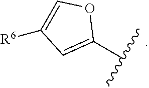

In some embodiments, R¹ is

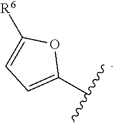

In some embodiments, R¹ is

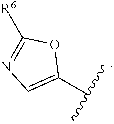

In some embodiments, R¹ is

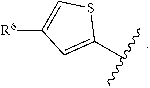

In some embodiments, R¹ is

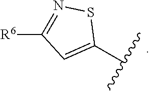

In some embodiments, R¹ is

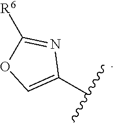

In some embodiments, R¹ is

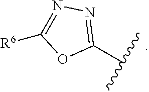

In some embodiments, R¹ is

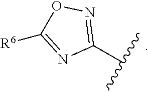

In some embodiments, R¹ is

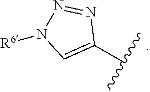

In some embodiments, R¹ is

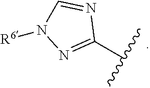

In some embodiments, R¹ is

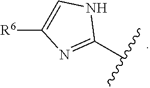

In some embodiments, R¹ is

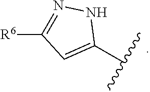

In some embodiments, R¹ is

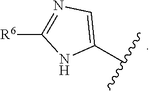

In some embodiments, R¹ is

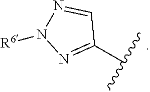

In some embodiments, R¹ is

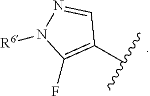

In some embodiments, R¹ is

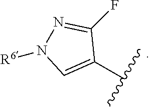

In some embodiments, R² is

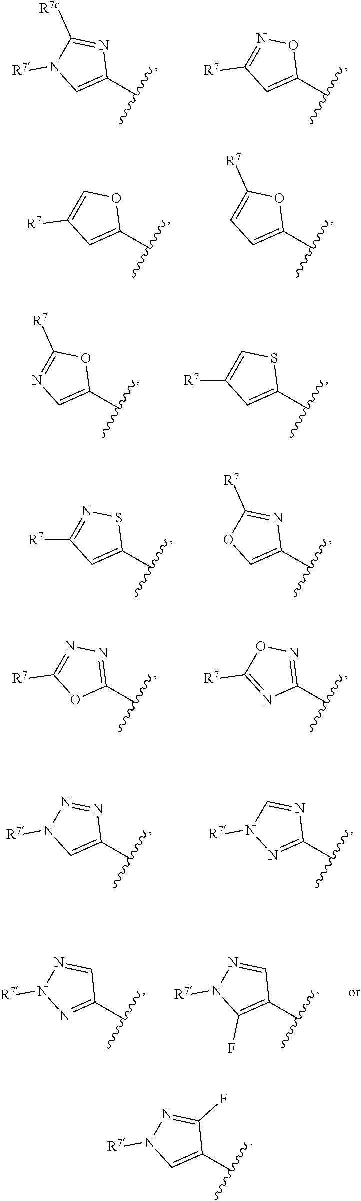

In some embodiments, R² is

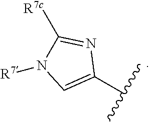

In some embodiments, R² is

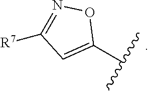

In some embodiments, R² is

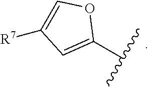

In some embodiments, R² is

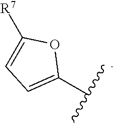

In some embodiments, R² is

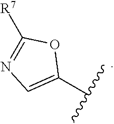

In some embodiments, R² is

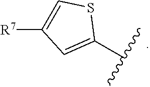

In some embodiments, R² is

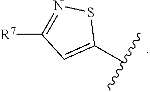

In some embodiments, R² is

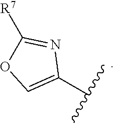

In some embodiments, R² is

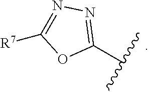

In some embodiments, R² is

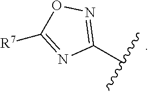

In some embodiments, R² is

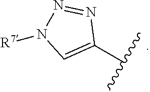

In some embodiments, R² is

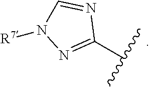

In some embodiments, R² is

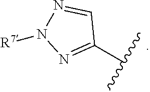

In some embodiments, R² is

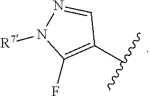

In some embodiments, R² is

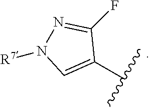

In some embodiments, R¹ and R² are selected from,

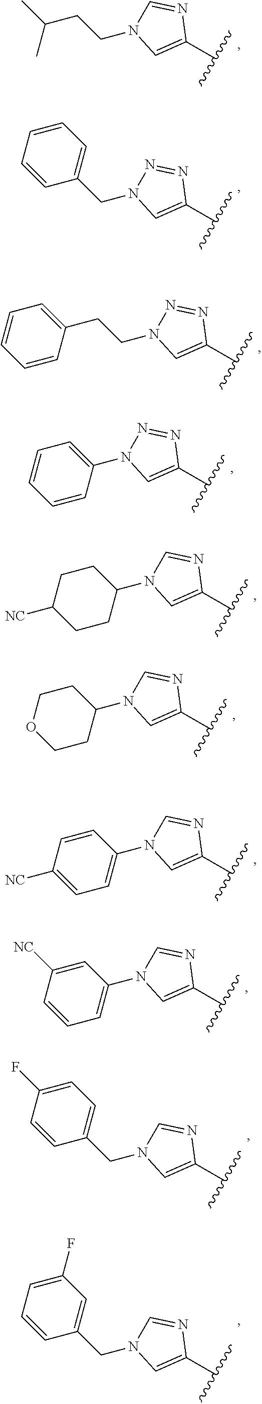

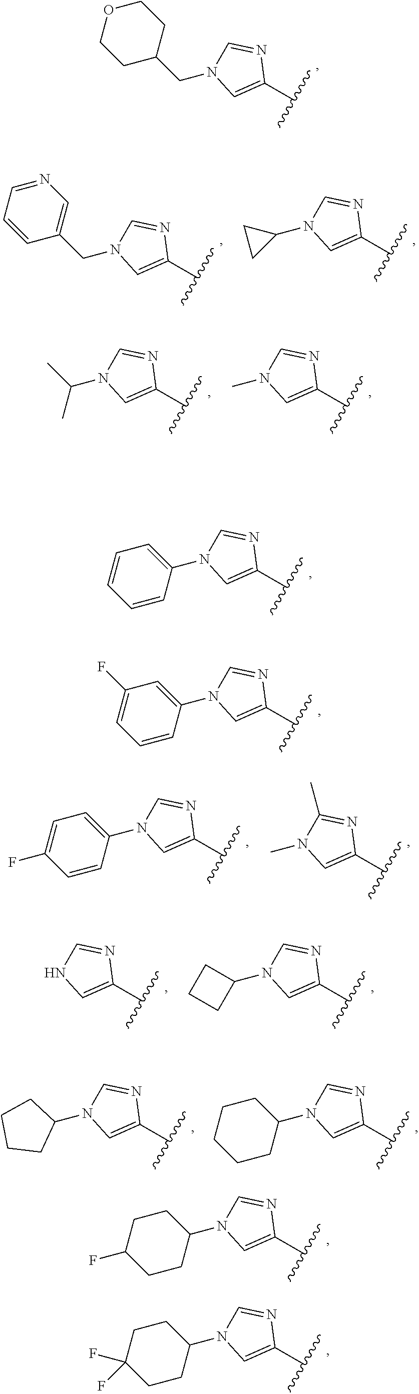

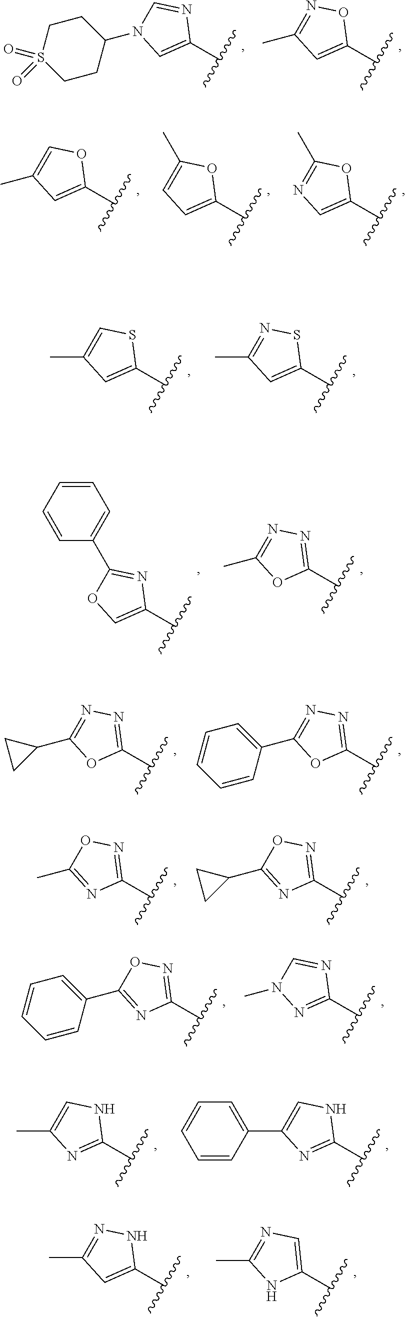

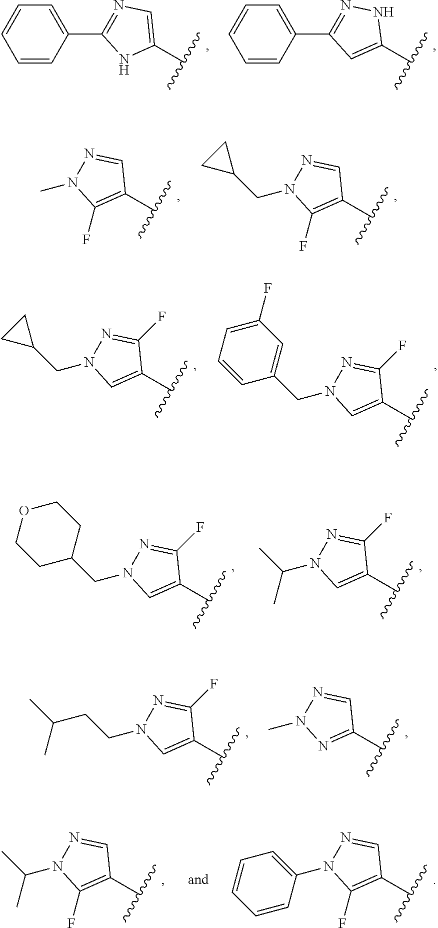

In some embodiments,
R¹ is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are substituted with one or more substituents independently selected from R⁶ and R^{6′}.

R² is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are substituted with one or more substituents independently selected from R⁷ and R^{7′}.

In some embodiments,
R¹ is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are substituted with one or more substituents independently selected from R⁶ and R^{6′}; and
R² is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are substituted with one or more substituents independently selected from R⁷ and R^{7′}.

In some embodiments, R¹ is

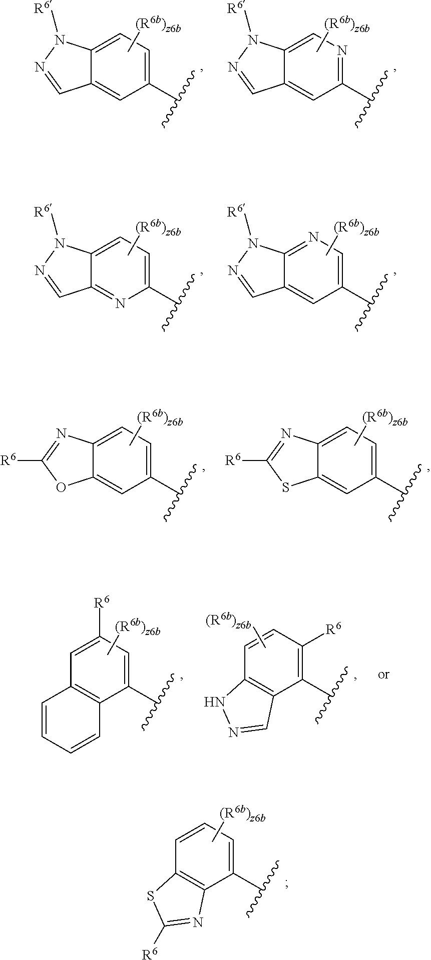

R¹ is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R⁵, —CH₂S(O)₂R⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
z6b is an integer from 0 to 4;
R² is

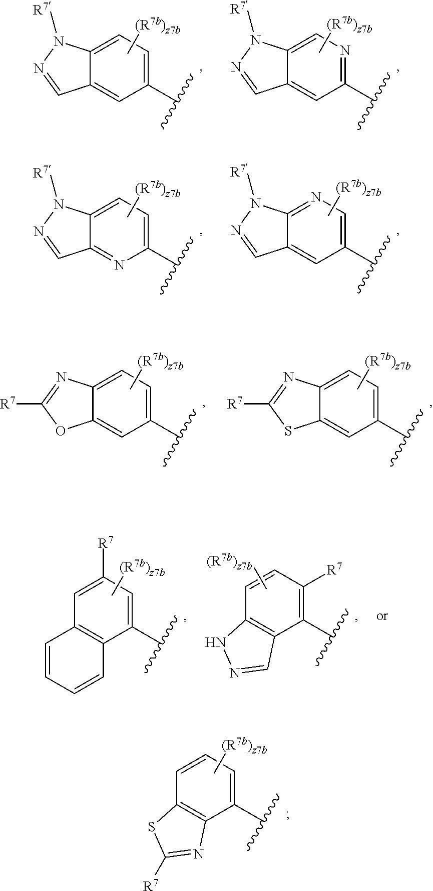

R^{7b} is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20b}; and
z7b is an integer from 0 to 4.

In some embodiments, R¹ and R² are selected from:

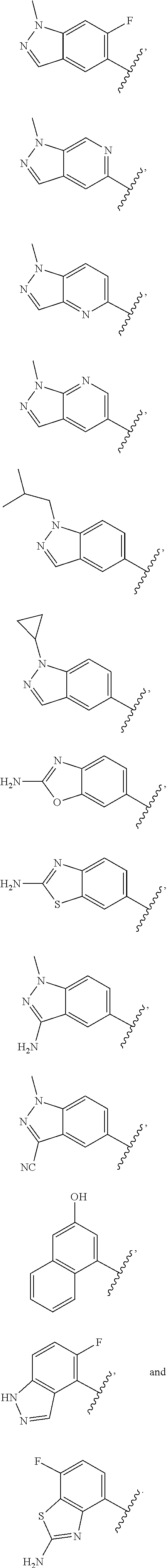

In some embodiments,
R¹ is pyrazolyl substituted with one or more substituents independently selected from R⁶ and R^{6′}; and
R² is pyrazolyl substituted with one or more substituents independently selected from R⁷ and R^{7′}.

In some embodiments,
R¹ is pyrazolyl substituted with one or more substituents independently selected from R^{6′}; and
R² is pyrazolyl substituted with one or more substituents independently selected from R^{7′}.

In some embodiments,
R¹ is selected from C₅₋₁₀cycloalkyl, 5 to 10 membered heterocycloalkyl, C₆aryl, and 5 to 10 membered heteroaryl, wherein C₅₋₁₀cycloalkyl, 5 to 10 membered heterocycloalkyl, C₆aryl, and 5 to 10 membered heteroaryl are substituted with one or more R⁶, wherein ring heteroatoms of the 5 to 10 membered heterocycloalkyl and 5 to 10 membered heteroaryl are independently selected from N, O, and S; and
each R⁶ is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀ aryl, —OR¹², —N(R¹²)(R¹³), —C(O)OR¹², —C(NR¹⁴)N(R¹²)(R¹³), —N(R¹⁴)S(O)₂R¹⁵, —C(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₁₀cycloalkyl, C₂₋₉heterocycloalkyl, and C₆₋₁₀aryl are optionally substituted with one, two, or three R²⁶.

In some embodiments, R¹ is selected from pyrrolyl, pyrazolyl, imidazolyl, triazolyl, pyrrolinyl, pyridinyl, tetrahydropyridinyl, piperidinyl, and piperazinyl, each of which is optionally substituted with one or more R⁶. In embodiments, R¹ is selected from pyrrolyl, pyrazolyl, imidazolyl, triazolyl, pyrrolinyl, pyridinyl, tetrahydropyridinyl, piperidinyl, and piperazinyl, each of which is optionally substituted with one or more substituents independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, —OR¹², —N(R¹²)(R¹³), —C(O)OR¹², —C(NR¹⁴)N(R¹²)(R¹³), —N(R¹⁴)S(O)₂R¹⁵, —C(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₁₀cycloalkyl, C₂₋₉heterocycloalkyl, and C₆₋₁₀aryl are optionally substituted with one, two, or three R²⁶.

In some embodiments, R¹ is selected from:

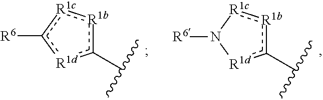

wherein R¹ is not N or N(R^{6c″}) when R^{1b} is C(R^{6b}) and R^{1d} is

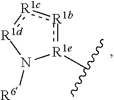

wherein R¹ is not a substituted pyrrolyl; and

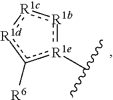

wherein R¹ is not a substituted imidazolyl wherein R^{1d} and R^{1e} are N;
R^{1b} is N, N(R^{6b″}), C(R^{6b}), or C(R^{6b})(R^{6b′});
R^{1c} is N, N(R^{6c″}), C(R^{6c}), or C(R^{6c})(R^{6c′})
R^{1d} is N, N(R^{6d″}), C(R^{6d}), or C(R^{6d})(R^{6d′});
R^{1e} is N, C, or C(R^{6e});
R^{6b}, R^{6b′}, R^{6c}, R^{6c′}, R^{6d}, R^{6d′}, and R^{6e} are independently selected from hydrogen, halogen, —CN, C₁₋₆alkyl, C₂₋₆alkenyl, C₂₋₆alkynyl, C_{3- 10}cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20a};
R^{6b″}, R^{6c″}, and R^{6d″} are independently selected from hydrogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
each R⁶ is independently selected from halogen, oxo, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁶; and
each R^{6′} is independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In some embodiments, R¹ is selected from

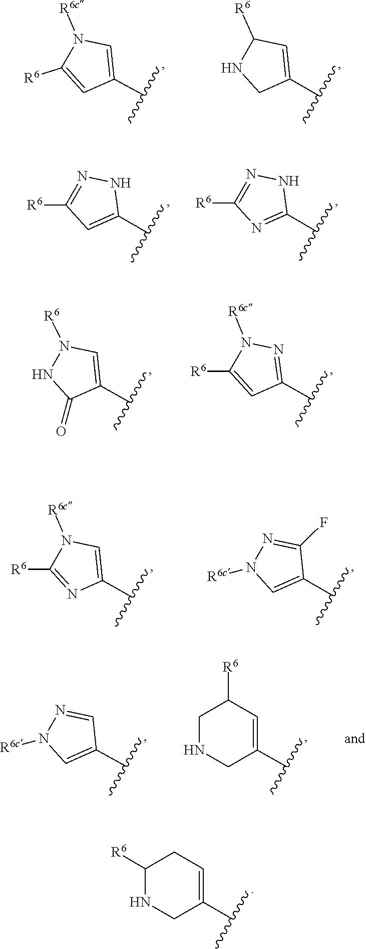

In some embodiments, R¹ is selected from

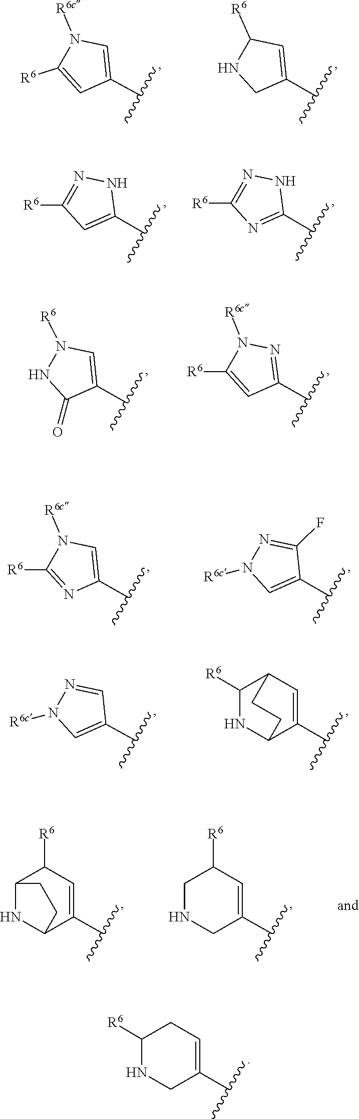

In some embodiments, R¹ is selected from

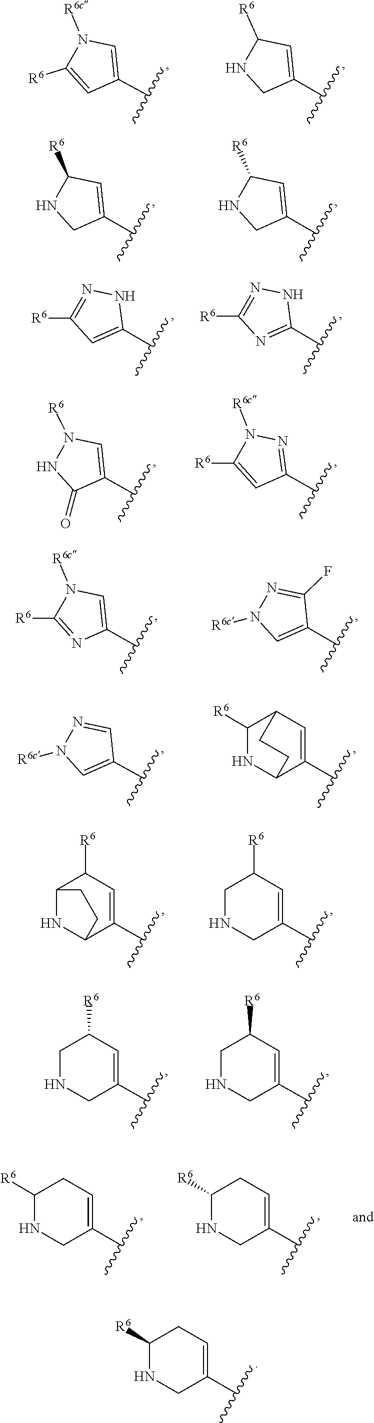

In some embodiments, R¹ is selected from

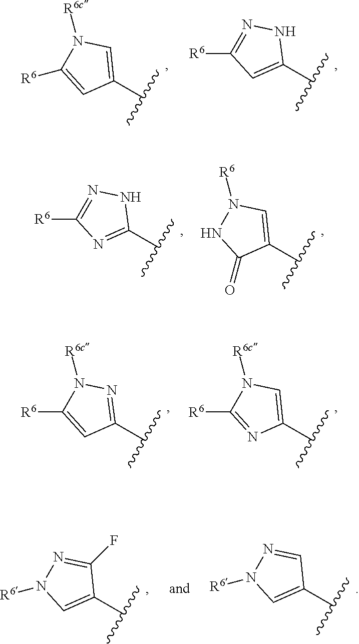

In some embodiments, R¹ is selected from

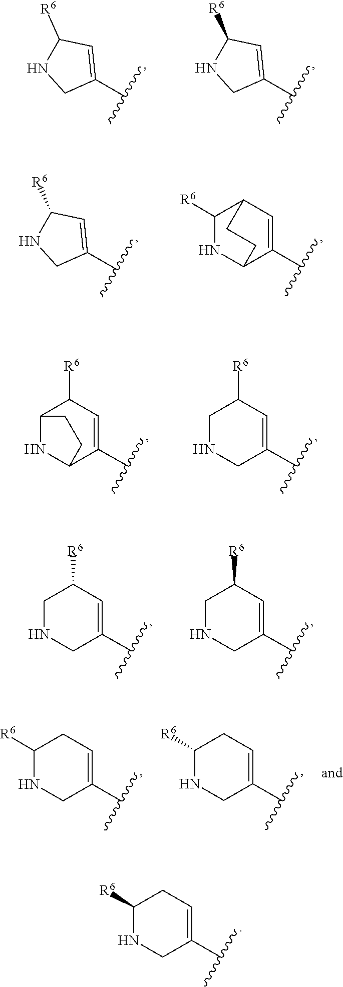

In some embodiments, R¹ is pyrazolyl substituted with one R^{6′}; and R² is pyrazolyl substituted with one R^{7′}. In some embodiments, R¹ is

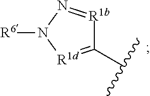

R^{1b} is O, S, N, N(R⁶″), C(R⁶), or C(R⁶)(R^{6′});
R^{1d} is O, S, N, N(R^{6d″}), C(R^{6d}), or C(R^{6d})(R^{6d′});
R^{6b}, R^{6b′}, R^{6d}, and R^{6d′} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20a};
R^{6b}″ and R^{6d″} are independently selected from hydrogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R² is

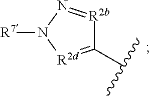

R^{2b} is O, S, N, N(R^{7b″}), C(R^{7b}), or C(R^{7b})(R^{7b′});
R^{2d} is O, S, N, N(R^{7d″}), C(R^{7d}), or C(R^{7d})(R^{7d′});
R^{7b}, R^{7b′}, R^{7d}, and R^{7d′} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20b};
R^{7b″} and R^{7d″} are independently selected from hydrogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b}; and
R^{6′} and R^{7′} are independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In some embodiments, R¹ is

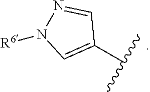

In some embodiments, R² is

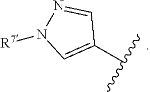

In some embodiments, R¹ is selected from C₅₋₁₂cycloalkyl, 5 to 12 membered heterocycloalkyl, C₆₋₁₂aryl, and 5 to 12 membered heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₅₋₁₂cycloalkyl, 5 to 12 membered heterocycloalkyl, C₆₋₁₂aryl, and 5 to 12 membered heteroaryl are optionally substituted with one or more substituents independently selected from R⁶ and R^{6′}.

In some embodiments, R¹ is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are optionally substituted with one or more substituents independently selected from R⁶ and R^{6′}. In some embodiments, R¹ is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are optionally substituted with one or more substituents independently selected from R^{6′}. In some embodiments, R¹ is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are optionally substituted with one or more substituents independently selected from R⁶. In some embodiments, R¹ is selected from C₅₋₁₂cycloalkyl, 5 to 12 membered heterocycloalkyl, C₆₋₁₂aryl, and 5 to 12 membered heteroaryl.

In some embodiments, R¹ is selected from —OR¹², —SR¹², —N(R¹²)(R¹³), and —C(O)OR¹².

In some embodiments, R^{1a} is selected from hydrogen, halogen, —CN, C₁₋₆alkyl, C₂₋₆alkenyl, and C₂₋₆alkynyl, wherein C₁₋₆ alkyl, C₂₋₆alkenyl, and C₂₋₆alkynyl are optionally substituted with one, two, or three R^{20a}. In some embodiments, R^{1a} is selected from halogen, —CN, C₁₋₆alkyl, C₂₋₆alkenyl, and C₂₋₆alkynyl, wherein C₁₋₆alkyl, C₂₋₆alkenyl, and C₂₋₆alkynyl are optionally substituted with one, two, or three R^{20a}. In some embodiments, R^{1a} is selected from C₁₋₆alkyl, C₂₋₆alkenyl, and C₂₋₆alkynyl, wherein C₁₋₆alkyl, C₂₋₆alkenyl, and C₂₋₆alkynyl are optionally substituted with one, two, or three R^{20a}. In some embodiments, R^{1a} is selected from C₁₋₆alkyl, C₂₋₆alkenyl, and C₂₋₆alkynyl.

In some embodiments, R⁶ is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉-heteroaryl, —OR¹², —N(R¹²)(R¹³), —C(O)R¹⁵, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶. In some embodiments, R⁶ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶. In some embodiments, R^{6′} is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In some embodiments, each R⁶ is independently selected from halogen, oxo, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶. In some embodiments, each R⁶ is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶. In some embodiments, each R⁶ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶. In some embodiments, each R⁶ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl. In some embodiments, each R⁶ is independently selected from halogen, —CN, —OR¹², —SR¹², —N(R¹²)(R¹³), —C(O)OR¹².

In some embodiments, each R^{6′} is independently selected from —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶. In some embodiments, each R^{6′} is independently selected from C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉-heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀ aryl, and C₁₋₉heteroaryl are optionally substituted with one, two, or three R²⁶. In some embodiments, each R^{6′} is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl. In some embodiments, each R^{6′} is independently C₁₋₆alkyl optionally substituted with one, two, or three R²⁶.

In some embodiments, R¹ is selected from C₅₋₁₂cycloalkyl, 5 to 12 membered heterocycloalkyl, C₇₋₁₂aryl, and 5 to 12 membered heteroaryl, wherein C₅₋₁₂cycloalkyl, 5 to 12 membered heterocycloalkyl, C₇₋₁₂aryl, and 5 to 12 membered heteroaryl are substituted with one or more substituents independently selected from R⁶ and R^{6′}, wherein ring heteroatoms of the 5 to 12 membered heterocycloalkyl and 5 to 12 membered heteroaryl are independently selected from C, N, O, and S. In some embodiments, R¹ is selected from C₅₋₁₂cycloalkyl, 5 to 12 membered heterocycloalkyl, C₇₋₁₂aryl, and 5 to 12 membered heteroaryl, wherein C₅₋₁₂ cycloalkyl, 5 to 12 membered heterocycloalkyl, C₇₋₁₂aryl, and 5 to 12 membered heteroaryl are substituted with one or more substituents independently selected from R⁶, wherein ring heteroatoms of the 5 to 12 membered heterocycloalkyl and 5 to 12 membered heteroaryl are independently selected from C, N, O, and S. In some embodiments, R¹ is selected from C₅₋₁₂cycloalkyl, 5 to 12 membered heterocycloalkyl, C₇₋₁₂aryl, and 5 to 12 membered heteroaryl, wherein C₅₋₁₂cycloalkyl, 5 to 12 membered heterocycloalkyl, C₇₋₁₂aryl, and 5 to 12 membered heteroaryl are substituted with one or more substituents independently selected from R^{6′}, wherein ring heteroatoms of the 5 to 12 membered heterocycloalkyl and 5 to 12 membered heteroaryl are independently selected from C, N, O, and S. In some embodiments, R¹ is selected from C₅₋₁₂cycloalkyl, 5 to 12 membered heterocycloalkyl, C₇. 1₂aryl, and 5 to 12 membered heteroaryl, wherein ring heteroatoms of the 5 to 12 membered heterocycloalkyl and 5 to 12 membered heteroaryl are independently selected from C, N, O, and S.

In some embodiments, R¹ is selected from C₅₋₁₂cycloalkyl, 5 to 12 membered partially unsaturated heterocycloalkyl, 6 to 12 membered saturated heterocycloalkyl, and 5 to 12 membered heteroaryl, wherein C₅₋₁₂cycloalkyl, 5 to 12 membered partially unsaturated heterocycloalkyl, 6 to 12 membered saturated heterocycloalkyl, and 5 to 12 membered heteroaryl are substituted with one or more substituents independently selected from R⁶ and R^{6′}, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S. In some embodiments, R¹ is selected from C₅₋₁₂cycloalkyl, 5 to 12 membered partially unsaturated heterocycloalkyl, 6 to 12 membered saturated heterocycloalkyl, and 5 to 12 membered heteroaryl, wherein C₅₋₁₂cycloalkyl, 5 to 12 membered partially unsaturated heterocycloalkyl, 6 to 12 membered saturated heterocycloalkyl, and 5 to 12 membered heteroaryl are substituted with one or more substituents independently selected from R⁶, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S. In some embodiments, R¹ is selected from C₅₋₁₂cycloalkyl, 5 to 12 membered partially unsaturated heterocycloalkyl, 6 to 12 membered saturated heterocycloalkyl, and 5 to 12 membered heteroaryl, wherein C₅₋₁₂cycloalkyl, 5 to 12 membered partially unsaturated heterocycloalkyl, 6 to 12 membered saturated heterocycloalkyl, and 5 to 12 membered heteroaryl are substituted with one or more substituents independently selected from R^{6′}, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S. In some embodiments, R¹ is selected from C₅₋₁₂cycloalkyl, 5 to 12 membered partially unsaturated heterocycloalkyl, 6 to 12 membered saturated heterocycloalkyl, and 5 to 12 membered heteroaryl, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S.

In embodiments, R¹ is

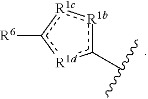

In embodiments, R¹ is

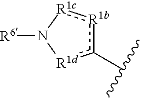

wherein R^{1c} is not N or N(R^{6e″}) when R^{1b} is C(R^{6b}) and R^{1d} is C(R^{6d}). In embodiments, R¹ is

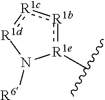

wherein R¹ is not a substituted pyrrolyl. In embodiments, R¹ is

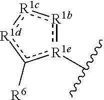

wherein R¹ is not a substituted imidazolyl wherein R^{1d} and R^{1e} are N. In embodiments, R¹ is fluoro-substituted pyrazolyl optionally further substituted with one, two, or three substituents independently selected from R⁶ and R^{6′}. In embodiments, R¹ is fluoro-substituted pyrazolyl optionally further substituted with one, two, or three substituents independently selected from R⁶. In embodiments, R¹ is fluoro-substituted pyrazolyl optionally further substituted with one, two, or three substituents independently selected from R^{6′}. In embodiments, R¹ is

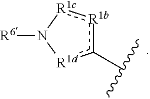

In embodiments, R¹ is

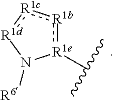

In embodiments, R¹ is

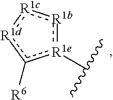

In some embodiments, R¹ is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are substituted with one or more substituents independently selected from R⁶ and R^{6′}. In some embodiments, R¹ is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are substituted with one or more substituents independently selected from R^{6′}. In some embodiments, R¹ is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are substituted with one or more substituents independently selected from R⁶ and R^{6′}. R¹ is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are substituted with one or more substituents independently selected from R⁶. In some embodiments, R¹ is selected from bicyclic C₄₋₁₂cycloalkyl, bicyclic C₂₋₁₁heterocycloalkyl, bicyclic C₇₋₁₂aryl, and bicyclic C₁₋₁₂heteroaryl.

In some embodiments, R¹ is pyrazolyl substituted with one or more substituents independently selected from R⁶ and R^{6′}. In some embodiments, R¹ is pyrazolyl substituted with one or more substituents independently selected from R⁶. In some embodiments, R¹ is pyrazolyl substituted with one or more substituents independently selected from R^{6′}. In some embodiments, R¹ is pyrazolyl substituted with one R^{6′}.

In some embodiments, R² is selected from C₅₋₁₂cycloalkyl, 5 to 12 membered heterocycloalkyl, C₆₋₁₂aryl, and 5 to 12 membered heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₅₋₁₂cycloalkyl, 5 to 12 membered heterocycloalkyl, C₆₋₁₂aryl, and 5 to 12 membered heteroaryl are optionally substituted with one or more substituents independently selected from R⁷ and R^{7′}. In some embodiments, R² is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are optionally substituted with one or more substituents independently selected from R⁷ and R^{7′}. In some embodiments, R² is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are optionally substituted with one or more substituents independently selected from R^{7′}. In some embodiments, R² is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are optionally substituted with one or more substituents independently selected from R⁷. In some embodiments, R² is selected from C₅₋₁₂cycloalkyl, 5 to 12 membered heterocycloalkyl, C₆₋₁₂aryl, and 5 to 12 membered heteroaryl. In some embodiments, R² is selected from —OR¹², —SR¹², —N(R¹²)(R¹³), and —C(O)OR¹².

In some embodiments, R² are independently selected from hydrogen, halogen, —CN, C₁₋₆alkyl, C₂₋₆alkenyl, and C₂₋₆alkynyl, wherein C₁₋₆alkyl, C₂₋₆alkenyl, and C₂₋₆alkynyl are optionally substituted with one, two, or three R^{20b}. In some embodiments, R² are independently selected from halogen, —CN, C₁₋₆alkyl, C₂₋₆alkenyl, and C₂₋₆alkynyl, wherein C₁₋₆alkyl, C₂₋₆alkenyl, and C₂₋₆alkynyl are optionally substituted with one, two, or three R^{20b}. In some embodiments, R^{2a} are independently selected from C₁₋₆alkyl, C₂₋₆alkenyl, and C₂₋₆alkynyl, wherein C₁₋₆alkyl, C₂₋₆alkenyl, and C₂₋₆alkynyl are optionally substituted with one, two, or three R^{20b}. In some embodiments, R^{2a} are independently selected from C₁₋₆alkyl, C₂₋₆alkenyl, and C₂₋₆alkynyl.

In some embodiments, R⁷ is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —N(R¹²)(R¹³), —C(O)R¹⁵, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶. In some embodiments, R⁷ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶. In some embodiments, R^{7′} is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In some embodiments, each R⁷ is independently selected from halogen, oxo, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R²⁶. In some embodiments, each R⁷ is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶. In some embodiments, each R⁷ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶. In some embodiments, each R⁷ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl. In some embodiments, each R⁷ is independently selected from halogen, —CN, —OR¹², —SR¹², —N(R¹²)(R¹³), —C(O)OR¹².

In some embodiments, each R^{7′} is independently selected from —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶. In some embodiments, each R^{7′} is independently selected from C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀ aryl, and C₁₋₉heteroaryl are optionally substituted with one, two, or three R²⁶. In some embodiments, each R^{7′} is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl. In some embodiments, each R^{7′} is independently C₁₋₆alkyl optionally substituted with one, two, or three R²⁶.

In some embodiments, R² is selected from C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl, wherein C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl are substituted with one or more substituents independently selected from R⁷ and R^{7′}, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S. In some embodiments, R² is selected from C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl, wherein C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl are substituted with one or more substituents independently selected from R^{7′}, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S. In some embodiments, R² is selected from C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl, wherein C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl are substituted with one or more substituents independently selected from R⁷, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S. In some embodiments, R² is selected from C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S.

In some embodiments, R² is selected from C₅₋₁₂cycloalkyl, 5 to 12 membered partially unsaturated heterocycloalkyl, 6 to 12 membered saturated heterocycloalkyl, and 5 to 12 membered heteroaryl, wherein C₅₋₁₂cycloalkyl, 5 to 12 membered partially unsaturated heterocycloalkyl, 6 to 12 membered saturated heterocycloalkyl, and 5 to 12 membered heteroaryl are substituted with one or more substituents independently selected from R⁷ and R^{7′}, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S. In some embodiments, R² is selected from C₅₋₁₂cycloalkyl, 5 to 12 membered partially unsaturated heterocycloalkyl, 6 to 12 membered saturated heterocycloalkyl, and 5 to 12 membered heteroaryl, wherein C₅₋₁₂cycloalkyl, 5 to 12 membered partially unsaturated heterocycloalkyl, 6 to 12 membered saturated heterocycloalkyl, and 5 to 12 membered heteroaryl are substituted with one or more substituents independently selected from R⁷, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S. In some embodiments, R² is selected from C₅₋₁₂cycloalkyl, 5 to 12 membered partially unsaturated heterocycloalkyl, 6 to 12 membered saturated heterocycloalkyl, and 5 to 12 membered heteroaryl, wherein C₅₋₁₂cycloalkyl, 5 to 12 membered partially unsaturated heterocycloalkyl, 6 to 12 membered saturated heterocycloalkyl, and 5 to 12 membered heteroaryl are substituted with one or more substituents independently selected from R^{7′}, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S. In some embodiments, R² is selected from C₅₋₁₂cycloalkyl, 5 to 12 membered partially unsaturated heterocycloalkyl, 6 to 12 membered saturated heterocycloalkyl, and 5 to 12 membered heteroaryl, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S.

In embodiments, R² is

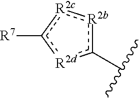

wherein R² is not a substituted imidazolyl wherein R^{2c} and R^{2d} are N. In embodiments, R² is

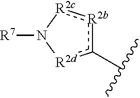

wherein R² is not a substituted pyrazolyl or pyrrolyl. In embodiments, R² is

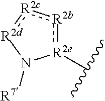

wherein R² is not a substituted pyrrolyl or imidazolyl. In embodiments, R² is

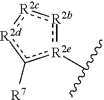

wherein R² is not a substituted imidazolyl wherein R^{2d} and R^{2e} are N, or R^{2b} and R^{2d} are N or NH. In embodiments, R² is fluoro-substituted pyrazolyl optionally further substituted with one, two, or three substituents independently selected from R⁷ and R^{7′}. In embodiments, R² is fluoro-substituted pyrazolyl optionally further substituted with one, two, or three substituents independently selected from R^{7′}. In embodiments, R² is fluoro-substituted pyrazolyl optionally further substituted with one, two, or three substituents independently selected from R^{7′}. In embodiments, R² is

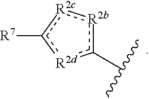

In embodiments, R² is

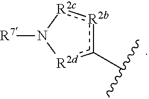

In embodiments, R² is

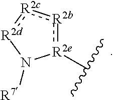

In embodiments, R² is

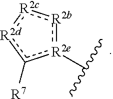

In some embodiments, R² is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are substituted with one or more substituents independently selected from R⁷ and R^{7′}. In some embodiments, R² is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are substituted with one or more substituents independently selected from R^{7′}. In some embodiments, R² is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are substituted with one or more substituents independently selected from R^{7′}. In some embodiments, R² is selected from bicyclic C₄₋₁₂cycloalkyl, bicyclic C₂₋₁₁heterocycloalkyl, bicyclic C₇₋₁₂aryl, and bicyclic C₁₋₁₂heteroaryl.

In some embodiments, R² is pyrazolyl substituted with one or more substituents independently selected from R⁷ and R^{7′}. In some embodiments, R² is pyrazolyl substituted with one or more substituents independently selected from R⁷. In some embodiments, R² is pyrazolyl substituted with one or more substituents independently selected from R^{7′}. In some embodiments, R² is pyrazolyl substituted with one R^{7′}.

In some embodiments, R⁶, R^{6′}, R⁷, and R^{7′} are independently selected from C₁₋₆alkyl,

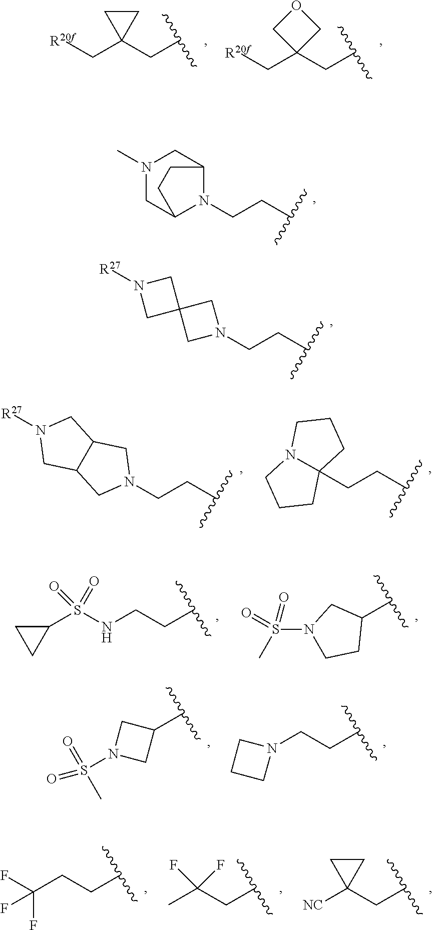

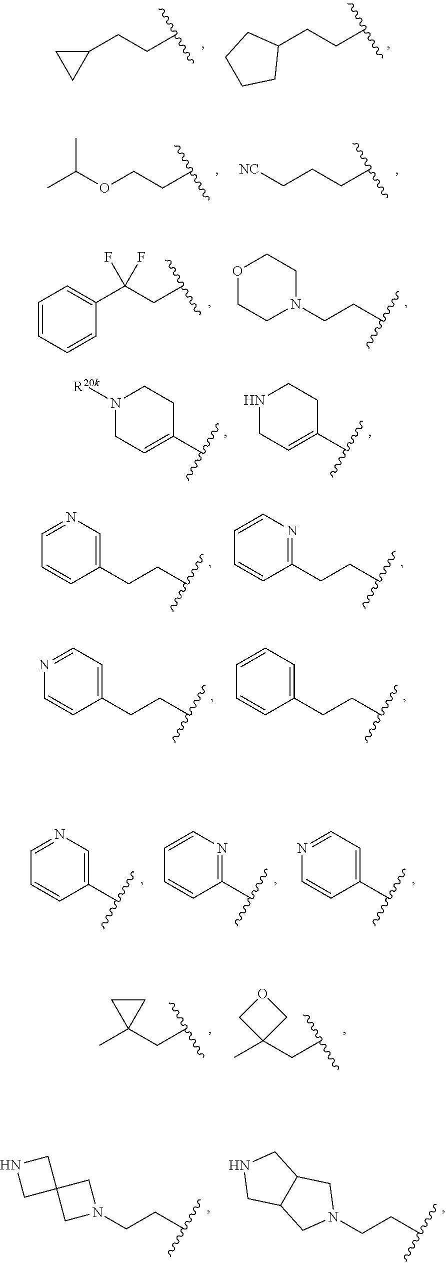

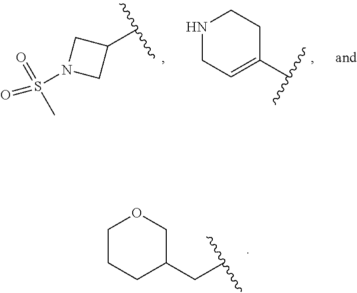

In some embodiments, R⁶, R^{6′}, R⁷, and R^{7′} are independently selected from

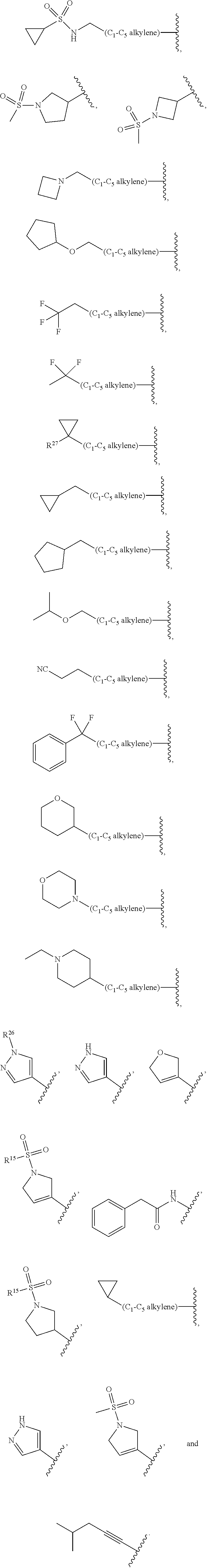

In some embodiments, R⁶, R^{6′}, R⁷, and R^{7′} are independently selected from

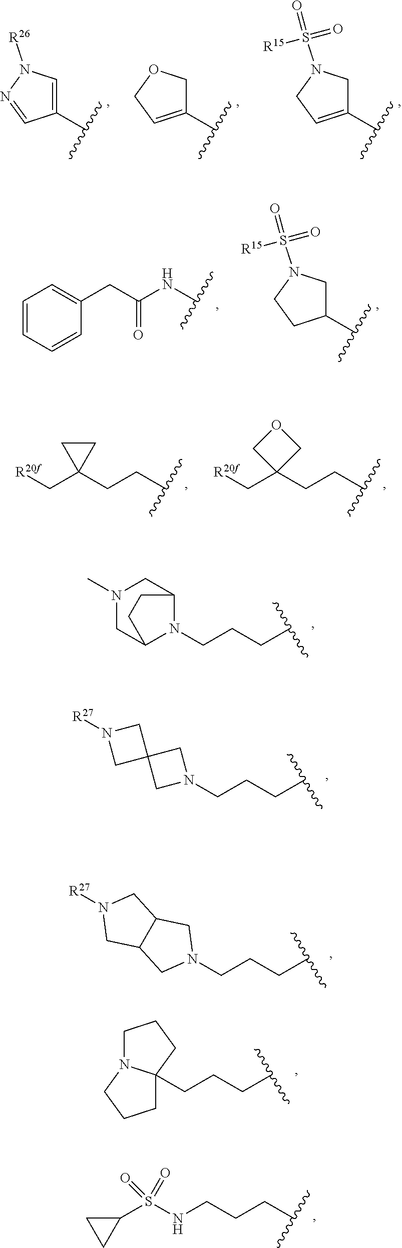

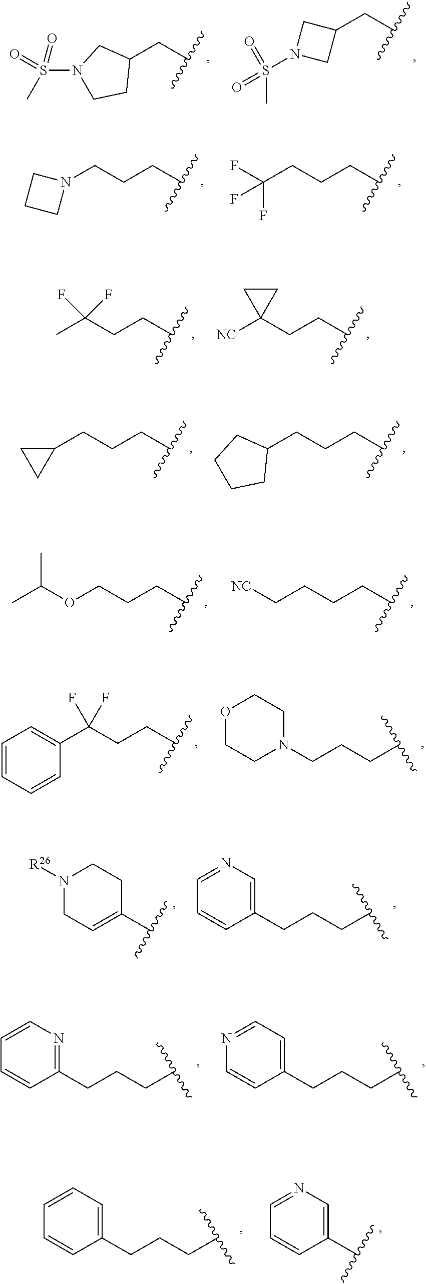

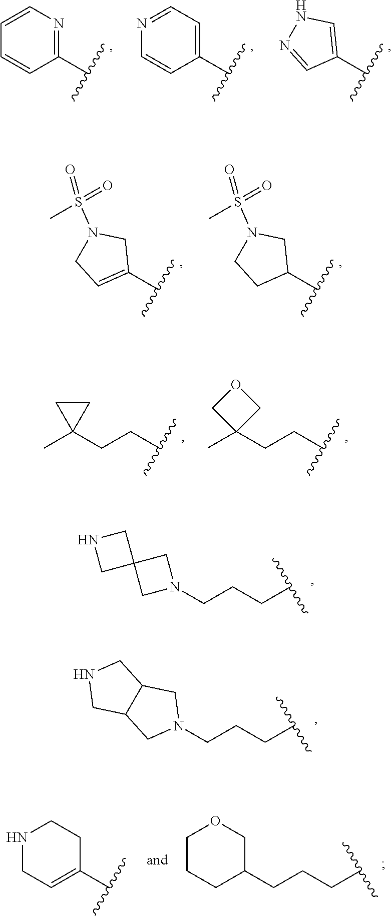

and
R²⁶ is independently selected from hydrogen, halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In some embodiments, R⁶, R^{6′}, R⁷, and R^{7′} are independently selected from

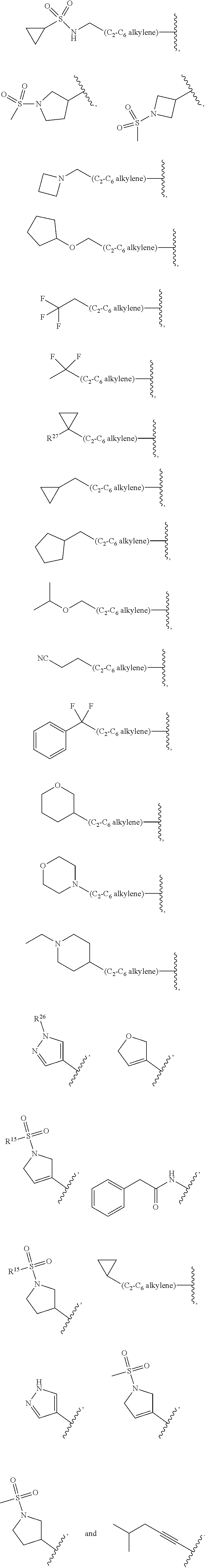

In some embodiments, R⁶, R^{6′}, R⁷, and R^{7′} are independently selected from

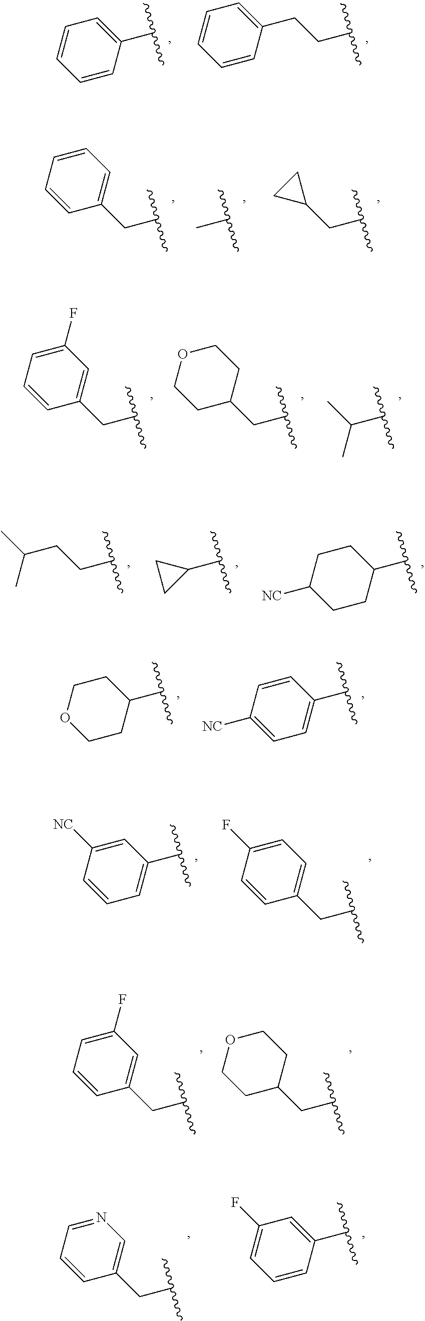

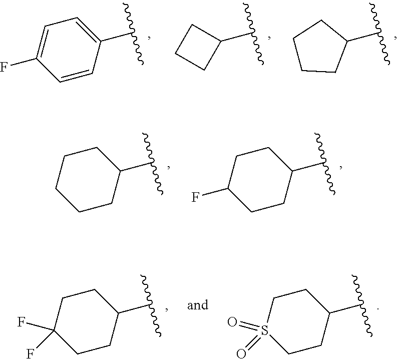

In some embodiments, R⁶ is independently selected from

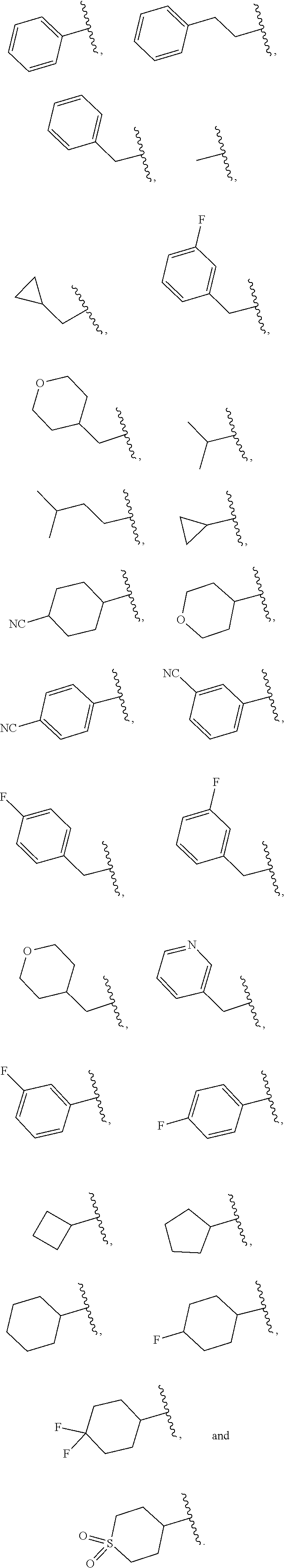

In some embodiments, R^{6′} is independently selected from

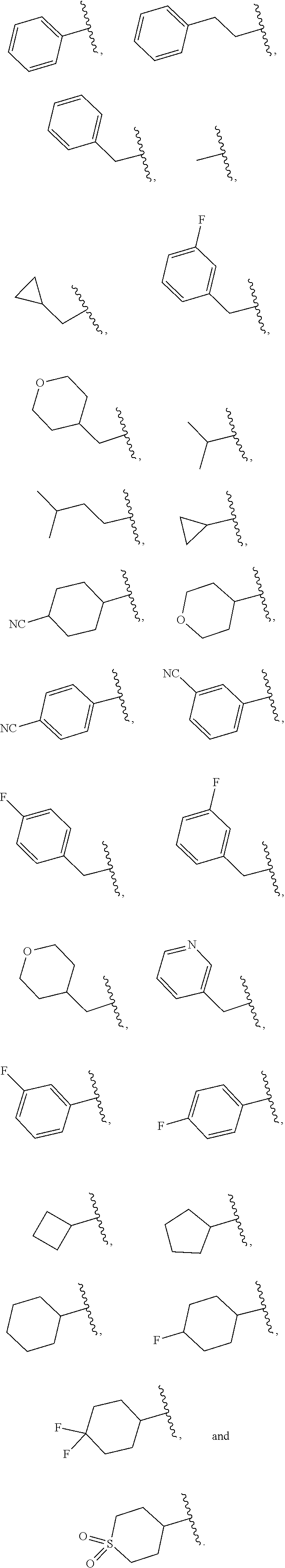

In some embodiment, R⁷ is independently selected from

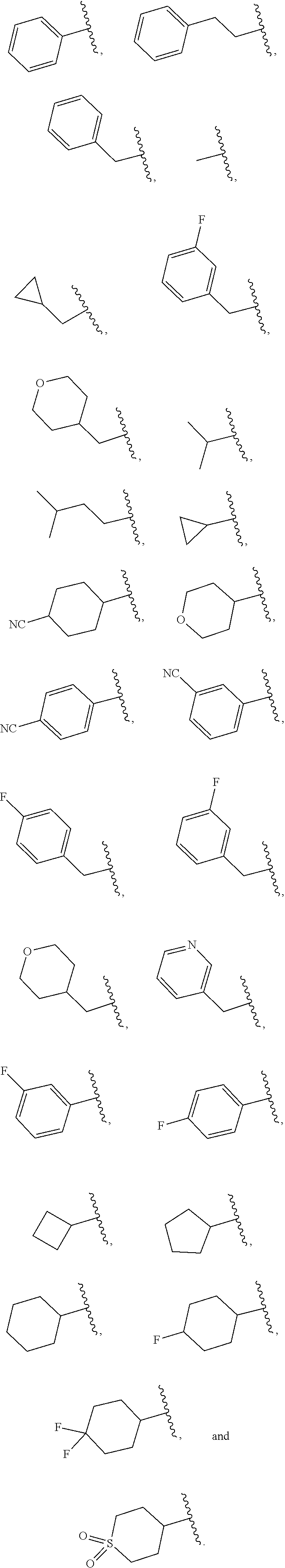

In some embodiments, R^{7′} is independently selected from

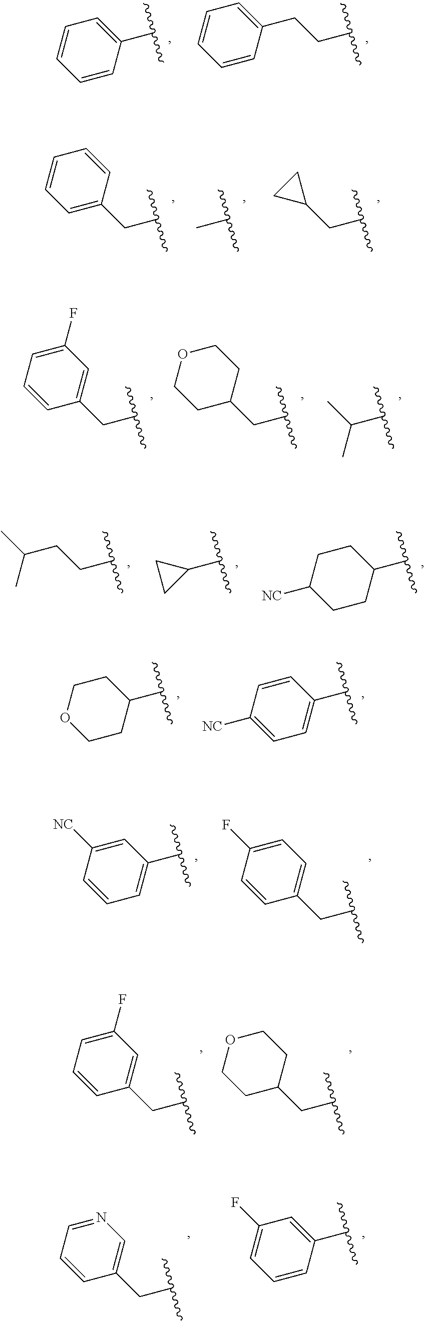

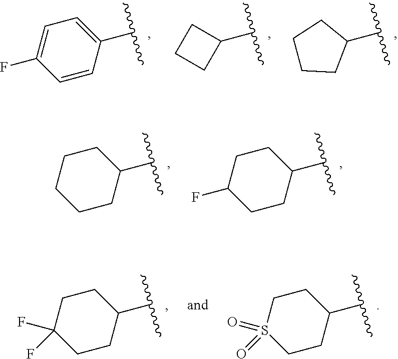

In some embodiments, R⁶ is —CH₂—R²⁶, wherein R²⁶ is independently selected from C₃₋₁₀cycloalkyl, C₂₋₉heterocycloalkyl, C₆₋₁₀aryl, C₁₋₉heteroaryl, wherein C₃₋₁₀cycloalkyl, C₂₋₉heterocycloalkyl, C₆₋₁₀aryl, and C₁₋₉heteroaryl are optionally substituted with one, two, or three R²⁷. In some embodiments, R⁶ is —CH₂—R²⁶, wherein R²⁶ is selected from C₆₋₁₀aryl and C₁₋₉heteroaryl, wherein C₆₋₁₀aryl and C₁₋₉heteroaryl are optionally substituted with one, two, or three R²⁷. In some embodiments, R⁶ is —CH₂—R²⁶, wherein R²⁶ is independently selected from C₃₋₁₀cycloalkyl, C₂₋₉heterocycloalkyl, C₆₋₁₀aryl, C₁₋₉heteroaryl, wherein C₃₋₁₀cycloalkyl, C₂₋₉ heterocycloalkyl, C₆₋₁₀aryl, and C₁₋₉heteroaryl are optionally substituted with one, two, or three R²⁷; and each R²⁷ is independently selected from halogen. In some embodiments, R⁶ is —CH₂—R²⁶, wherein R²⁶ is independently selected from C₃₋₁₀cycloalkyl, C₂₋₉ heterocycloalkyl, C₆₋₁₀aryl, C₁₋₉heteroaryl, wherein C₃₋₁₀cycloalkyl, C₂₋₉heterocycloalkyl, C₆₋₁₀aryl, and C₁₋₉heteroaryl are optionally substituted with one R²⁷; and R²⁷ is independently selected from halogen.

In some embodiments, R^{6′} is —CH₂—R²⁶, wherein R²⁶ is independently selected from C₃₋₁₀cycloalkyl, C₂₋₉heterocycloalkyl, C₆₋₁₀aryl, C₁₋₉heteroaryl, wherein C₃₋₁₀cycloalkyl, C₂₋₉heterocycloalkyl, C₆₋₁₀aryl, and C₁₋₉heteroaryl are optionally substituted with one, two, or three R²⁷. In some embodiments, R^{6′} is —CH₂—R²⁶, wherein R²⁶ is selected from C₆₋₁₀aryl and C₁₋₉heteroaryl, wherein C₆₋₁₀aryl and C₁₋₉heteroaryl are optionally substituted with one, two, or three R²⁷. In some embodiments, R^{6′} is —CH₂—R²⁶, wherein R²⁶ is independently selected from C₃₋₁₀cycloalkyl, C₂₋₉heterocycloalkyl, C₆₋₁₀aryl, C₁₋₉heteroaryl, wherein C₃₋₁₀cycloalkyl, C₂₋₉ heterocycloalkyl, C₆₋₁₀aryl, and C₁₋₉heteroaryl are optionally substituted with one, two, or three R²⁷; and each R²⁷ is independently selected from halogen. In some embodiments, R^{6′} is —CH₂—R²⁶, wherein R²⁶ is independently selected from C₃₋₁₀cycloalkyl, C₂₋₉ heterocycloalkyl, C₆₋₁₀aryl, C₁₋₉heteroaryl, wherein C₃₋₁₀cycloalkyl, C₂₋₉heterocycloalkyl, C₆₋₁₀aryl, and C₁₋₉heteroaryl are optionally substituted with one R²⁷; and R²⁷ is independently selected from halogen.

In some embodiments, R⁷ is —CH₂—R²⁶, wherein R²⁶ is independently selected from C₃₋₁₀cycloalkyl, C₂₋₉heterocycloalkyl, C₆₋₁₀aryl, C₁₋₉heteroaryl, wherein C₃₋₁₀cycloalkyl, C₂₋₉heterocycloalkyl, C₆₋₁₀aryl, and C₁₋₉heteroaryl are optionally substituted with one, two, or three R²⁷. In some embodiments, R⁷ is —CH₂—R²⁶, wherein R²⁶ is selected from C₆₋₁₀aryl and C₁₋₉heteroaryl, wherein C₆₋₁₀aryl and C₁₋₉heteroaryl are optionally substituted with one, two, or three R²⁷. In some embodiments, R⁷ is —CH₂—R²⁶, wherein R²⁶ is independently selected from C₃₋₁₀cycloalkyl, C₂₋₉heterocycloalkyl, C₆₋₁₀aryl, C₁₋₉heteroaryl, wherein C₃₋₁₀cycloalkyl, C₂₋₉ heterocycloalkyl, C₆₋₁₀aryl, and C₁₋₉heteroaryl are optionally substituted with one, two, or three R²⁷; and each R²⁷ is independently selected from halogen. In some embodiments, R⁷ is —CH₂—R²⁶, wherein R²⁶ is independently selected from C₃₋₁₀cycloalkyl, C₂₋₉ heterocycloalkyl, C₆₋₁₀aryl, C₁₋₉heteroaryl, wherein C₃₋₁₀cycloalkyl, C₂₋₉heterocycloalkyl, C₆₋₁₀aryl, and C₁₋₉heteroaryl are optionally substituted with one R²⁷; and R²⁷ is independently selected from halogen.

In some embodiments, R^{7′} is —CH₂—R²⁶, wherein R²⁶ is independently selected from C₃₋₁₀cycloalkyl, C₂₋₉heterocycloalkyl, C₆₋₁₀aryl, C₁₋₉heteroaryl, wherein C₃₋₁₀cycloalkyl, C₂₋₉heterocycloalkyl, C₆₋₁₀aryl, and C₁₋₉heteroaryl are optionally substituted with one, two, or three R²⁷. In some embodiments, R^{7′} is —CH₂—R²⁶, wherein R²⁶ is selected from C₆₋₁₀aryl and C₁₋₉heteroaryl, wherein C₆₋₁₀aryl and C₁₋₉heteroaryl are optionally substituted with one, two, or three R²⁷. In some embodiments, R^{7′} is —CH₂—R²⁶, wherein R²⁶ is independently selected from C₃₋₁₀cycloalkyl, C₂₋₉heterocycloalkyl, C₆₋₁₀aryl, C₁₋₉heteroaryl, wherein C₃₋₁₀cycloalkyl, C₂₋₉ heterocycloalkyl, C₆₋₁₀aryl, and C₁₋₉heteroaryl are optionally substituted with one, two, or three R²⁷; and each R²⁷ is independently selected from halogen. In some embodiments, R^{7′} is —CH₂—R²⁶, wherein R²⁶ is independently selected from C₃₋₁₀cycloalkyl, C₂₋₉heterocycloalkyl, C₆₋₁₀aryl, C₁₋₉heteroaryl, wherein C₃₋₁₀cycloalkyl, C₂₋₉heterocycloalkyl, C₆₋₁₀aryl, and C₁₋₉heteroaryl are optionally substituted with one R²⁷; and R²⁷ is independently selected from halogen.

In some embodiments, at least one of X, Y, Z, W¹, and W² is N.

In some embodiments, each R^{20a}, R^{20b}, R^{20c}, R^{20d}, R^{20e}, R^{20f}, R^{20h}, R²⁰ⁱ, R^{20j}, R^{20k}, R^{20l}, R^{20m}, R²⁰ⁿ, and R^{20o} is independently selected from oxo, —CN, halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

In an aspect is provided a compound of Formula (IV), or a pharmaceutically acceptable salt or solvate thereof:

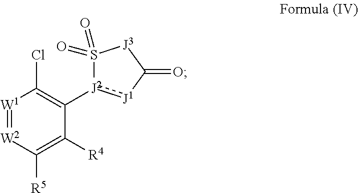

wherein:
W¹ is C(R¹) and W² is C(R^{2a}), or W¹ is C(Ria) and W² is C(R²);
R¹ is selected from C₅₋₁₂cycloalkyl, 5 to 12 membered heterocycloalkyl, C₆₋₁₂aryl, 6 to 12 membered heteroaryl, and a five membered heteroaryl having the formula

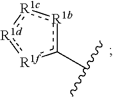

wherein C₅₋₁₂cycloalkyl, 5 to 12 membered heterocycloalkyl, C₆₋₁₂aryl, and 6 to 12 membered heteroaryl, are optionally substituted with one or more substituents independently selected from R⁶ and R^{6′} wherein ring heteroatoms of the 5 to 12 membered heterocycloalkyl and 6 to 12 membered heteroaryl are independently selected from N, O, and S;
R^{1b} is O, S, N, N(R^{6b″}), C(R^{6b}), or C(R^{6b})(R^{6b′});
R^{1c} is O, S, N, N(R^{6c″}), C(R^{6c}), or C(R^{6c})(R^{6c′})
R^{1d} is O, S, N, N(R^{6d″}), C(R^{6d}), or C(R^{6d})(R^{6d′});
R^{1f} is O, S, N, N(R^{6f″}), C(R^{6f}), or C(R^{6f})(R^{6f′}
R^{6b}, R^{6b′}, R^{6c}, R^{6c′}, R^{6d}, R^{6d′}, R^{6f}, and R^{6f′} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R^{6b″}, R^{6c″}, R^{6d″}, and R^{6f″} are independently selected from hydrogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R^{1a} is selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20a};
R² is selected from C₅₋₁₂cycloalkyl, 5 to 12 membered heterocycloalkyl, C₆₋₁₂aryl, 6 to 12 membered heteroaryl, and a five membered heteroaryl having the formula

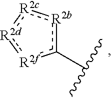

wherein C₅₋₁₂cycloalkyl, 5 to 12 membered heterocycloalkyl, C₆₋₁₂aryl, and 6 to 12 membered heteroaryl are optionally substituted with one or more substituents independently selected from R⁷ and R^{7′}, wherein ring heteroatoms of the 5 to 12 membered heterocycloalkyl and 6 to 12 membered heteroaryl are independently selected from N, O, and S;
R^{2b} is O, S, N, N(R^{7b″}), C(R^{7b}), or C(R^{7b})(R^{7′});
R^{2c} is O, S, N, N(R^{7c″}), C(R^{7c}), or C(R^{7c})(R^{7c′})
R^{2d} is O, S, N, N(R^{7d″}), C(R^{7d}), or C(R^{7d})(R^{7d′});
R^{2f} is O, S, N, N(R^{7f″}), C(R^{7f}), or C(R^{7f})(R^{7f′})
R^{7b}, R^{7b′}, R^{7c}, R^{7c′}, R^{7d}, R^{7d′}, R^{7f}, and R^{7f} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20b};
R^{b7″}, R^{7c″}, R^{7d″}, and R^{7f′} are independently selected from hydrogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20b}; and
R^{2a} are independently selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20b};
R⁴ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
each R⁶ and R⁷ are independently selected from halogen, oxo, —CN, C₁₋₆alkyl, C₂₋₆alkenyl, C₂₋₆alkynyl, C₃₋₆cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁶;
each R^{6′} and R^{7′} are independently selected from —CN, C₁₋₆alkyl, C₂₋₆alkenyl, C₂₋₆alkynyl, C₃₋₆cycloalkyl, C₂₋₉heterocycloalkyl, C₆₋₁₀ aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁶;
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉ heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉ heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉ heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉ heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20l};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₉heteroalkyl, C₂₋₉heterocycloalkyl, C₆₋₁₀aryl, and C₁₋₉heteroaryl are optionally substituted with one, two, or three R^{20m};
each R¹⁶ is independently selected from —C₁₋₆alkylene-OP(O)(OR^{16a})(OR⁶Ib) and —P(O)(OR^{16a})(OR⁶Ib); wherein the C₁₋₆alkylene is optionally substituted with one, two, or three R²ⁿ;
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20o};
each R^{20a}, R^{20b}, R^{20d}, R^{20e}, R^{20f}, R^{20h}, R²⁰ⁱ, R^{20j}, R^{20k}, R^{20l}, R^{20m}, R²⁰ⁿ, and R^{20o} are each independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl optionally substituted with one or more R²⁶, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉ heteroaryl;
each R²⁶ is independently selected from oxo, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁷;
each R²⁷ is independently selected from oxo, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20f};
[symbol] indicates a single or double bond such that all valences are satisfied.

In some embodiments, a compound described herein, or a pharmaceutically acceptable salt or solvate thereof, has the structure of Formula (IVa):

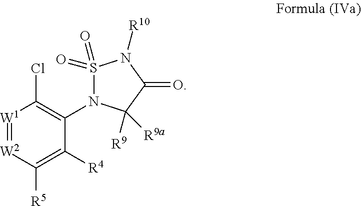

In some embodiments, a compound described herein, or a pharmaceutically acceptable salt or solvate thereof, has the structure of Formula (IVb):

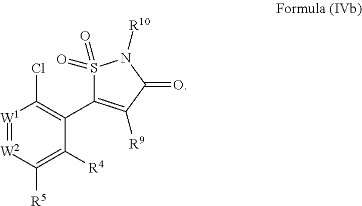

In some embodiments, a compound described herein, or a pharmaceutically acceptable salt or solvate thereof, has the structure of Formula (IVc):

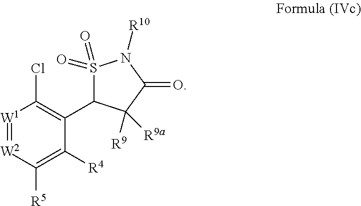

In some embodiments, of a compound of formula IV, IVa, IVb, or IVc described herein, or a pharmaceutically acceptable salt or solvate thereof, R¹ is

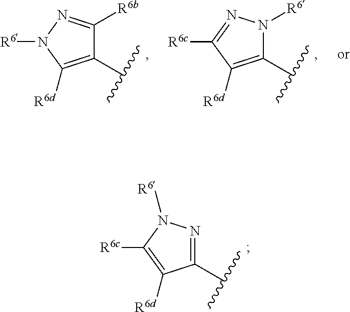

R^{6b}, R^{6c}, and R^{6d} are independently selected from hydrogen, halogen, —CN, C₁₋₆alkyl, C₂₋₆alkenyl, C₂₋₆alkynyl, C₃₋₆cycloalkyl, C₂₋₉ heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20a}; R² is

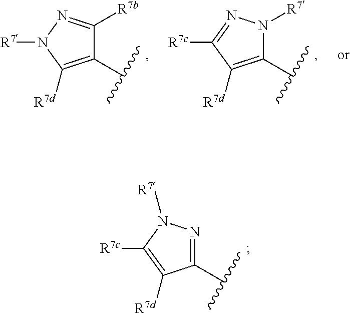

R^{7b}, R^{7c}, and R^{7d} are independently selected from hydrogen, halogen, —CN, C₁₋₆alkyl, C₂₋₆alkenyl, C₂₋₆alkynyl, C₃₋₆cycloalkyl, C₂₋₉ heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b};
R^{6′} and R^{7′} are independently selected from —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁶.

In some embodiments, of a compound of formula IV, IVa, IVb, or IVc described herein, or a pharmaceutically acceptable salt or solvate thereof, R⁴ is selected from hydrogen, halogen, —OR¹², and C₁₋₆alkyl optionally substituted with one, two, or three R^{20d}. In some embodiments, of a compound of formula IV, IVa, IVb, or IVc described herein, or a pharmaceutically acceptable salt or solvate thereof, R⁴ is —OH.

In some embodiments, of a compound of formula IV, IVa, IVb, or IVc described herein, or a pharmaceutically acceptable salt or solvate thereof, R⁵ is selected from hydrogen, halogen, and C₁₋₆alkyl optionally substituted with one, two, or three R^{20e}. In some embodiments, of a compound of formula IV, IVa, IVb, or IVc described herein, or a pharmaceutically acceptable salt or solvate thereof, R⁵ is hydrogen.

In some embodiments, of a compound of formula IV, IVa, IVb, or IVc described herein, or a pharmaceutically acceptable salt or solvate thereof, R¹ is

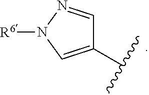

In some embodiments, of a compound of formula IV, IVa, IVb, or IVc described herein, or a pharmaceutically acceptable salt or solvate thereof, R² is

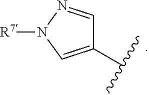

In some embodiments, of a compound of formula IV, IVa, IVb, or IVc described herein, or a pharmaceutically acceptable salt or solvate thereof, R⁶, R^{6′}, R⁷, and R^{7′} are independently selected from

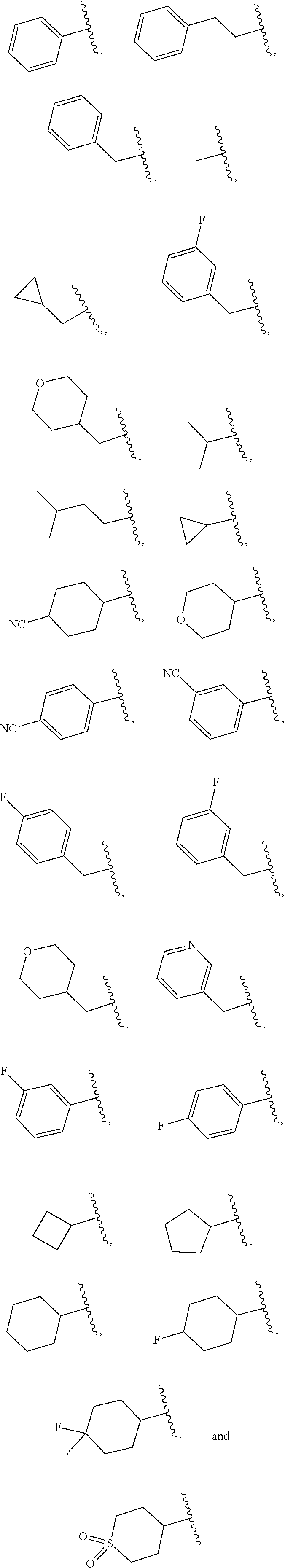

each R²¹ is independently selected from H, C₁₋₆alkyl, C₁₋₆haloalkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl.

In embodiments of a compound of Formula (IV), W¹ is C(R¹) and W² is C(R^{2a}). In embodiments of a compound of Formula (IV), W¹ is C(R^{1a}) and W² is C(R²).

In embodiments of a compound of Formula (IV), R¹ is C₅₋₁₂cycloalkyl optionally substituted with one or more substituents independently selected from R⁶ and R^{6′}. In embodiments of a compound of Formula (IV), R¹ is 5 to 12 membered heterocycloalkyl optionally substituted with one or more substituents independently selected from R⁶ and R^{6′}, wherein ring heteroatoms of the 5 to 12 membered heterocycloalkyl are independently selected from N, O, and S. In embodiments of a compound of Formula (IV), R¹ is C₆₋₁₂aryl optionally substituted with one or more substituents independently selected from R⁶ and R^{6′}. In embodiments of a compound of Formula (IV), R¹ is 6 to 12 membered heteroaryl optionally substituted with one or more substituents independently selected from R⁶ and R^{6′}, wherein ring heteroatoms of the 6 to 12 membered heteroaryl are independently selected from N, O, and S. In embodiments of a compound of Formula (IV), R¹ is five membered heteroaryl having the formula

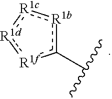

In embodiments of a compound of Formula (IV), R^{1b} is O. In embodiments of a compound of Formula (IV), R^{1b} is S. In embodiments of a compound of Formula (IV), R^{1b} is N. In embodiments of a compound of Formula (IV), R^{1b} is N(R^{6b″}). In embodiments of a compound of Formula (IV), R^{1b} is C(R^{6b}). In embodiments of a compound of Formula (IV), R^{1b} is C(R^{6b})(R^{6b′}). In embodiments of a compound of Formula (IV), R^{1c} is O. In embodiments of a compound of Formula (IV), R^{1c} is S. In embodiments of a compound of Formula (IV), R^{1c} is N. In embodiments of a compound of Formula (IV), R^{1c} is N(R^{6c″}). In embodiments of a compound of Formula (IV), R^{1c} is C(R^{6e}). In embodiments of a compound of Formula (IV), R^{1c} is C(R^{6e})(R^{6e′}). In embodiments of a compound of Formula (IV), R^{1d} is O. In embodiments of a compound of Formula (IV), R^{1d} is S. In embodiments of a compound of Formula (IV), R^{1d} is N. In embodiments of a compound of Formula (IV), R^{1d} is N(R^{6d″}). In embodiments of a compound of Formula (IV), R^{1d} is C(R^{6d}). In embodiments of a compound of Formula (IV), R^{1d} is C(R^{6d})(R^{6d′}). In embodiments of a compound of Formula (IV), R^{1f} is O. In embodiments of a compound of Formula (IV), R^{1f} is S. In embodiments of a compound of Formula (IV), R^{1f} is N. In embodiments of a compound of Formula (IV), R^{1f} is N(R^{6f′}). In embodiments of a compound of Formula (IV), R^{1f} is C(R^{6f}). In embodiments of a compound of Formula (IV), R^{1f} is C(R^{6f})(R^{6′}). In embodiments of a compound of Formula (IV), R^{6b}, R^{6b′}, R^{6c}, R^{6c′}, R^{6d}, R^{6d′}, R^{6f}, and R^{6f′} are independently selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl —OR¹², —SR¹², —N(R1²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20a}. In embodiments of a compound of Formula (IV), R^{6b″}, R^{6c″}, R^{6d″}, and R^{6f′} are independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl —OR¹², wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20a}.

In embodiments of a compound of Formula (IV), R¹ is C₅₋₁₂cycloalkyl substituted with one or more substituents independently selected from R⁶ and R^{6′}. In embodiments of a compound of Formula (IV), R¹ is 5 to 12 membered heterocycloalkyl substituted with one or more substituents independently selected from R⁶ and R^{6′}, wherein ring heteroatoms of the 5 to 12 membered heterocycloalkyl are independently selected from N, O, and S. In embodiments of a compound of Formula (IV), R¹ is C₆₋₁₂aryl substituted with one or more substituents independently selected from R⁶ and R^{6′}. In embodiments of a compound of Formula (IV), R¹ is 6 to 12 membered heteroaryl substituted with one or more substituents independently selected from R⁶ and R^{6′}, wherein ring heteroatoms of the 6 to 12 membered heteroaryl are independently selected from N, O, and S.

In embodiments of a compound of Formula (IV), R¹ is C₅₋₁₂cycloalkyl substituted with one or more substituents independently selected from R^{6′}. In embodiments of a compound of Formula (IV), R¹ is 5 to 12 membered heterocycloalkyl substituted with one or more substituents independently selected from R^{6′}, wherein ring heteroatoms of the 5 to 12 membered heterocycloalkyl are independently selected from N, O, and S. In embodiments of a compound of Formula (IV), R¹ is C₆₋₁₂aryl substituted with one or more substituents independently selected from R^{6′}. In embodiments of a compound of Formula (IV), R¹ is 6 to 12 membered heteroaryl substituted with one or more substituents independently selected from R^{6′}, wherein ring heteroatoms of the 6 to 12 membered heteroaryl are independently selected from N, O, and S.

In embodiments of a compound of Formula (IV), R¹ is C₅₋₁₂cycloalkyl substituted with one or more substituents independently selected from R⁶. In embodiments of a compound of Formula (IV), R¹ is 5 to 12 membered heterocycloalkyl substituted with one or more substituents independently selected from R⁶, wherein ring heteroatoms of the 5 to 12 membered heterocycloalkyl are independently selected from N, O, and S. In embodiments of a compound of Formula (IV), R¹ is C₆₋₁₂aryl substituted with one or more substituents independently selected from R⁶. In embodiments of a compound of Formula (IV), R¹ is 6 to 12 membered heteroaryl substituted with one or more substituents independently selected from R⁶, wherein ring heteroatoms of the 6 to 12 membered heteroaryl are independently selected from N, O, and S.

In embodiments of a compound of Formula (IV), R¹ is C₅₋₁₂cycloalkyl. In embodiments of a compound of Formula (IV), R¹ is 5 to 12 membered heterocycloalkyl, wherein ring heteroatoms of the 5 to 12 membered heterocycloalkyl are independently selected from N, O, and S. In embodiments of a compound of Formula (IV), R¹ is C₆₋₁₂aryl. In embodiments of a compound of Formula (IV), R¹ is 6 to 12 membered heteroaryl, wherein ring heteroatoms of the 6 to 12 membered heteroaryl are independently selected from N, O, and S.

In embodiments of a compound of Formula (IV), R^{1a} is hydrogen.

In embodiments of a compound of Formula (IV), R² is C₅₋₁₂cycloalkyl optionally substituted with one or more substituents independently selected from R⁷ and R^{7′}. In embodiments of a compound of Formula (IV), R² is 5 to 12 membered heterocycloalkyl optionally substituted with one or more substituents independently selected from R⁷ and R^{7′} wherein ring heteroatoms of the 5 to 12 membered heterocycloalkyl are independently selected from N, O, and S. In embodiments of a compound of Formula (IV), R² is C₆₋₁₂aryl optionally substituted with one or more substituents independently selected from R⁷ and R^{∂′}. In embodiments of a compound of Formula (IV), R² is 6 to 12 membered heteroaryl optionally substituted with one or more substituents independently selected from R⁷ and R^{7′} wherein ring heteroatoms of the 6 to 12 membered heteroaryl are independently selected from N, O, and S. In embodiments of a compound of Formula (IV), R² is a five membered heteroaryl having the formula

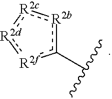

In embodiments of a compound of Formula (IV), R^{2b} is O. In embodiments of a compound of Formula (IV), R^{2b} is S. In embodiments of a compound of Formula (IV), R^{2b} is N. In embodiments of a compound of Formula (IV), R^{2b} is N(R^{7b″}). In embodiments of a compound of Formula (IV), R^{2b} is C(R^{7b}). In embodiments of a compound of Formula (IV), R^{2b} is C(R^{7b})(R^{7b′}). In embodiments of a compound of Formula (IV), R^{2c} is O. In embodiments of a compound of Formula (IV), R^{2c} is S. In embodiments of a compound of Formula (IV), R^{2c} is N. In embodiments of a compound of Formula (IV), R^{2c} is N(R^{7c″}). In embodiments of a compound of Formula (IV), R^{2c} is C(R^{7c}). In embodiments of a compound of Formula (IV), R^{2c} is C(R^{7c})(R^{7c′}). In embodiments of a compound of Formula (IV), R^{2d} is O. In embodiments of a compound of Formula (IV), R^{2d} is S. In embodiments of a compound of Formula (IV), R^{2d} is N. In embodiments of a compound of Formula (IV), R^{2d} is N(R^{7d″}). In embodiments of a compound of Formula (IV), R^{2d} is C(R^{7d}). In embodiments of a compound of Formula (IV), R^{2d} is C(R^{7d})(R^{7d′}). In embodiments of a compound of Formula (IV), R² is O. In embodiments of a compound of Formula (IV), R^{2f} is S. In embodiments of a compound of Formula (IV), R² is N. In embodiments of a compound of Formula (IV), R^{2f} is N(R^{7f′}). In embodiments of a compound of Formula (IV), R^{2f} is C(R^{f}). In embodiments of a compound of Formula (IV), R^{2f} is C(R^{7f})(R^{7′}).

In embodiments of a compound of Formula (IV), R² is C₅₋₁₂cycloalkyl substituted with one or more substituents independently selected from R⁷ and R^{7′}. In embodiments of a compound of Formula (IV), R² is 5 to 12 membered heterocycloalkyl substituted with one or more substituents independently selected from R⁷ and R^{7′}, wherein ring heteroatoms of the 5 to 12 membered heterocycloalkyl are independently selected from N, O, and S. In embodiments of a compound of Formula (IV), R² is C₆₋₁₂aryl substituted with one or more substituents independently selected from R⁷ and R^{7′}. In embodiments of a compound of Formula (IV), R² is 6 to 12 membered heteroaryl substituted with one or more substituents independently selected from R⁷ and R^{7′}, wherein ring heteroatoms of the 6 to 12 membered heteroaryl are independently selected from N, O, and S.

In embodiments of a compound of Formula (IV), R² is C₅₋₁₂cycloalkyl substituted with one or more substituents independently selected from R^{7′}. In embodiments of a compound of Formula (IV), R² is 5 to 12 membered heterocycloalkyl substituted with one or more substituents independently selected from R^{7′}, wherein ring heteroatoms of the 5 to 12 membered heterocycloalkyl are independently selected from N, O, and S. In embodiments of a compound of Formula (IV), R² is C₆₋₁₂aryl substituted with one or more substituents independently selected from R^{7′}. In embodiments of a compound of Formula (IV), R² is 6 to 12 membered heteroaryl substituted with one or more substituents independently selected from R^{7′}, wherein ring heteroatoms of the 6 to 12 membered heteroaryl are independently selected from N, O, and S.

In embodiments of a compound of Formula (IV), R² is C₅₋₁₂cycloalkyl substituted with one or more substituents independently selected from R⁷. In embodiments of a compound of Formula (IV), R² is 5 to 12 membered heterocycloalkyl substituted with one or more substituents independently selected from R⁷, wherein ring heteroatoms of the 5 to 12 membered heterocycloalkyl are independently selected from N, O, and S. In embodiments of a compound of Formula (IV), R² is C₆₋₁₂aryl substituted with one or more substituents independently selected from R⁷. In embodiments of a compound of Formula (IV), R² is 6 to 12 membered heteroaryl substituted with one or more substituents independently selected from R⁷, wherein ring heteroatoms of the 6 to 12 membered heteroaryl are independently selected from N, O, and S.

In embodiments of a compound of Formula (IV), R² is C₅₋₁₂cycloalkyl. In embodiments of a compound of Formula (IV), R² is 5 to 12 membered heterocycloalkyl, wherein ring heteroatoms of the 5 to 12 membered heterocycloalkyl are independently selected from N, O, and S. In embodiments of a compound of Formula (IV), R² is C₆₋₁₂aryl. In embodiments of a compound of Formula (IV), R² is 6 to 12 membered heteroaryl, wherein ring heteroatoms of the 6 to 12 membered heteroaryl are independently selected from N, O, and S.

In embodiments of a compound of Formula (IV), R⁴ is selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉ heterocycloalkyl, C₆₋₁₀aryl, and C₁₋₉heteroaryl are optionally substituted with one, two, or three R^{20d}.

In some embodiments, of a compound of formula IV, IVa, IVb, or IVc described herein, R¹ is

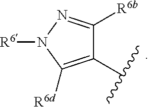

In some embodiments, of a compound of formula IV, IVa, IVb, or IVc described herein, R¹ is

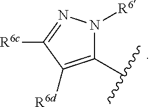

In some embodiments, of a compound of formula IV, IVa, IVb, or IVc described herein, R¹ is

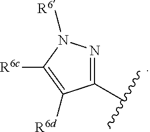

In some embodiments, of a compound of formula IV, IVa, IVb, or IVc described herein, R² is

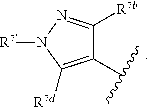

In some embodiments, of a compound of formula IV, IVa, IVb, or IVc described herein, R² is

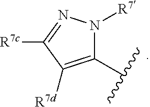

In some embodiments, of a compound of formula IV, IVa, IVb, or IVc described herein, R² is

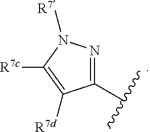

In an aspect is provided a compound of Formula (V), or a pharmaceutically acceptable salt or solvate thereof:

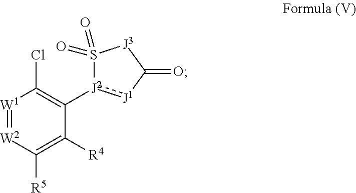

wherein:
W¹ is C(R¹) and W² is C(R^{2a}), or W¹ is C(R^{1a}) and W² is C(R²); R¹ is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl, are optionally substituted with one or more substituents independently selected from R⁶ and R^{6′}, wherein ring heteroatoms of the 5 to 12 membered heterocycloalkyl and 5 to 12 membered heteroaryl are independently selected from N, O, and S;
R^{1a} is selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20a};
R² is selected from C₅₋₁₂cycloalkyl, 5 to 12 membered heterocycloalkyl, C₆₋₁₂aryl, and 5 to 12 membered heteroaryl, wherein C₅₋₁₂ cycloalkyl, 5 to 12 membered heterocycloalkyl, C₆₋₁₂aryl, and 5 to 12 membered heteroaryl are optionally substituted with one or more substituents independently selected from R⁷ and R^{7′}, wherein ring heteroatoms of the 5 to 12 membered heterocycloalkyl and 5 to 12 membered heteroaryl are independently selected from N, O, and S;
R² are independently selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20b};
R⁴ is selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
each R⁶ and R⁷ are independently selected from halogen, oxo, —CN, C₁₋₆alkyl, C₂₋₆alkenyl, C₂₋₆alkynyl, C₃₋₆cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁶;
each R^{6′} and R^{7′} are independently selected from —CN, C₁₋₆alkyl, C₂₋₆alkenyl, C₂₋₆alkynyl, C₃₋₆cycloalkyl, C₂₋₉heterocycloalkyl, C₆₋₁₀ aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁶;
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R¹ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉ heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉ heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉ heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20l};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl are optionally substituted with one, two, or three R^{20m};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R²ⁿ;
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R²⁰;
each R^{20a}, R^{20b}, R^{20d}, R^{20e}, R^{20f}, R^{20h}, R²⁰ⁱ, R^{20j}, R^{20k}, R^{20l}, R^{20m}, R²⁰ⁿ, and R^{20o} are each independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl optionally substituted with one or more R²⁶, C₁₋₆haloalkyl, C₁₋₆alkoxy, C₁₋₉haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉ heteroaryl;
each R²⁶ is independently selected from oxo, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁷;
each R²⁷ is independently selected from oxo, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl optionally substituted with one, two, or three R^{20f}; [symbol] indicates a single or double bond such that all valences are satisfied.

In embodiments, a compound described herein, or a pharmaceutically acceptable salt or solvate thereof, has the structure of Formula (Va):

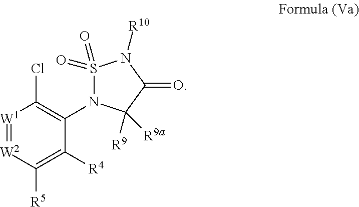

In embodiments, a compound described herein, or a pharmaceutically acceptable salt or solvate thereof, has the structure of Formula (Vb):

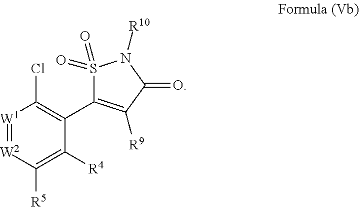

In embodiments, a compound described herein, or a pharmaceutically acceptable salt or solvate thereof, has the structure of Formula (Vc):

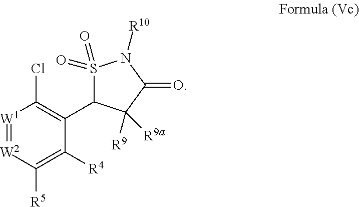

In embodiments, R¹ is

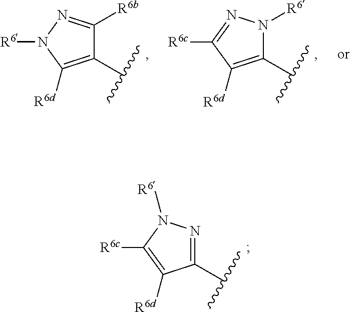

wherein
R^{6b}, R^{6c}, and R^{6d} are independently selected from hydrogen, halogen, —CN, C₁₋₆alkyl, C₂₋₆alkenyl, C₂₋₆alkynyl, C₃₋₆cycloalkyl, C₂₋₉ heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20a};
R² is

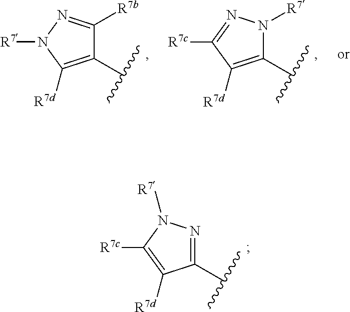

R^{7b}, R^{7c}, and R^{7d} are independently selected from hydrogen, halogen, —CN, C₁₋₆alkyl, C₂₋₆alkenyl, C₂₋₆alkynyl, C₃₋₆cycloalkyl, C₂₋₉ heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20b};
R^{6′} and R^{7′} are independently selected from —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁶.

In embodiments of a compound of Formula (V) described herein, or a pharmaceutically acceptable salt or solvate thereof, R⁴ is selected from hydrogen, halogen, —OR¹², and C₁₋₆alkyl optionally substituted with one, two, or three R^{20d}. In embodiments of a compound of Formula (V) described herein, or a pharmaceutically acceptable salt or solvate thereof, R⁴ is —OH.

In embodiments of a compound of Formula (V) described herein, or a pharmaceutically acceptable salt or solvate thereof, R⁵ is selected from hydrogen, halogen, and C₁₋₆alkyl optionally substituted with one, two, or three R^{20e}. In embodiments of a compound of Formula (V) described herein, or a pharmaceutically acceptable salt or solvate thereof, R⁵ is hydrogen. In embodiments of a compound of Formula (V) described herein, or a pharmaceutically acceptable salt or solvate thereof, R⁵ is —OH.

In embodiments of a compound of Formula (V) described herein, or a pharmaceutically acceptable salt or solvate thereof, R¹ is

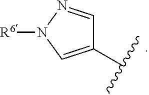

In embodiments of a compound of Formula (V) described herein, or a pharmaceutically acceptable salt or solvate thereof, R² is

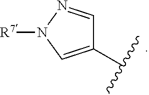

In embodiments of a compound of Formula (V) described herein, or a pharmaceutically acceptable salt or solvate thereof, R⁶, R^{6′}, R⁷, and R^{7′} are independently selected from

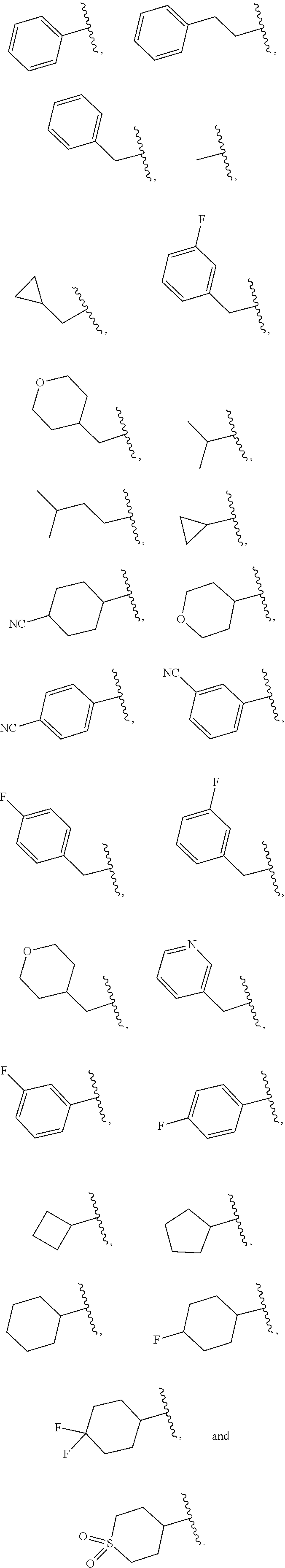

In embodiments of a compound of Formula (V), W¹ is C(R¹) and W² is C(R^{2a}). In embodiments of a compound of Formula (V), W¹ is C(R^{1a}) and W² is C(R²).

In embodiments of a compound of Formula (V), R¹ is C₅₋₁₂cycloalkyl optionally substituted with one or more substituents independently selected from R⁶ and R^{6′}. In embodiments of a compound of Formula (V), R¹ is 5 to 12 membered heterocycloalkyl optionally substituted with one or more substituents independently selected from R⁶ and R^{6′}, wherein ring heteroatoms of the 5 to 12 membered heterocycloalkyl are independently selected from N, O, and S. In embodiments of a compound of Formula (V), R¹ is C₆₋₁₂aryl optionally substituted with one or more substituents independently selected from R⁶ and R^{6′}. In embodiments of a compound of Formula (V), R¹ is 5 to 12 membered heteroaryl optionally substituted with one or more substituents independently selected from R⁶ and R^{6′}, wherein ring heteroatoms of the 5 to 12 membered heteroaryl are independently selected from N, O, and S.

In embodiments of a compound of Formula (V), R¹ is C₅₋₁₂cycloalkyl substituted with one or more substituents independently selected from R⁶ and R^{6′}. In embodiments of a compound of Formula (V), R¹ is 5 to 12 membered heterocycloalkyl substituted with one or more substituents independently selected from R⁶ and R^{6′}, wherein ring heteroatoms of the 5 to 12 membered heterocycloalkyl are independently selected from N, O, and S. In embodiments of a compound of Formula (V), R¹ is C₆₋₁₂aryl substituted with one or more substituents independently selected from R⁶ and R^{6′}. In embodiments of a compound of Formula (V), R¹ is 5 to 12 membered heteroaryl substituted with one or more substituents independently selected from R⁶ and R^{6′}, wherein ring heteroatoms of the 5 to 12 membered heteroaryl are independently selected from N, O, and S.

In embodiments of a compound of Formula (V), R¹ is C₅₋₁₂cycloalkyl substituted with one or more substituents independently selected from R^{6′}. In embodiments of a compound of Formula (V), R¹ is 5 to 12 membered heterocycloalkyl substituted with one or more substituents independently selected from R^{6′}, wherein ring heteroatoms of the 5 to 12 membered heterocycloalkyl are independently selected from N, O, and S. In embodiments of a compound of Formula (V), R¹ is C₆₋₁₂aryl substituted with one or more substituents independently selected from R^{6′}. In embodiments of a compound of Formula (V), R¹ is 5 to 12 membered heteroaryl substituted with one or more substituents independently selected from R^{6′}, wherein ring heteroatoms of the 5 to 12 membered heteroaryl are independently selected from N, O, and S.

In embodiments of a compound of Formula (V), R¹ is C₅₋₁₂cycloalkyl substituted with one or more substituents independently selected from R⁶. In embodiments of a compound of Formula (V), R¹ is 5 to 12 membered heterocycloalkyl substituted with one or more substituents independently selected from R⁶, wherein ring heteroatoms of the 5 to 12 membered heterocycloalkyl are independently selected from N, O, and S. In embodiments of a compound of Formula (V), R¹ is C₆₋₁₂aryl substituted with one or more substituents independently selected from R⁶. In embodiments of a compound of Formula (V), R¹ is 5 to 12 membered heteroaryl substituted with one or more substituents independently selected from R⁶, wherein ring heteroatoms of the 5 to 12 membered heteroaryl are independently selected from N, O, and S.

In embodiments of a compound of Formula (V), R¹ is C₅₋₁₂cycloalkyl. In embodiments of a compound of Formula (V), R¹ is 5 to 12 membered heterocycloalkyl, wherein ring heteroatoms of the 5 to 12 membered heterocycloalkyl are independently selected from N, O, and S. In embodiments of a compound of Formula (V), R¹ is C₆₋₁₂aryl. In embodiments of a compound of Formula (V), R¹ is 5 to 12 membered heteroaryl, wherein ring heteroatoms of the 5 to 12 membered heteroaryl are independently selected from N, O, and S.

In embodiments of a compound of Formula (V), R^{1a} is hydrogen.

In embodiments of a compound of Formula (V), R² is C₅₋₁₂cycloalkyl optionally substituted with one or more substituents independently selected from R⁷ and R^{7′}. In embodiments of a compound of Formula (V), R² is 5 to 12 membered heterocycloalkyl optionally substituted with one or more substituents independently selected from R⁷ and R^{7′} wherein ring heteroatoms of the 5 to 12 membered heterocycloalkyl are independently selected from N, O, and S. In embodiments of a compound of Formula (V), R² is C₆₋₁₂aryl optionally substituted with one or more substituents independently selected from R⁷ and R^{7′}. In embodiments of a compound of Formula (V), R² is 5 to 12 membered heteroaryl optionally substituted with one or more substituents independently selected from R⁷ and R^{7′} wherein ring heteroatoms of the 5 to 12 membered heteroaryl are independently selected from N, O, and S.

In embodiments of a compound of Formula (V), R² is C₅₋₁₂cycloalkyl substituted with one or more substituents independently selected from R⁷ and R^{7′}. In embodiments of a compound of Formula (V), R² is 5 to 12 membered heterocycloalkyl substituted with one or more substituents independently selected from R⁷ and R^{7′}, wherein ring heteroatoms of the 5 to 12 membered heterocycloalkyl are independently selected from N, O, and S. In embodiments of a compound of Formula (V), R² is C₆₋₁₂aryl substituted with one or more substituents independently selected from R⁷ and R^{7′}. In embodiments of a compound of Formula (V), R² is 5 to 12 membered heteroaryl substituted with one or more substituents independently selected from R⁷ and R^{7′}, wherein ring heteroatoms of the 5 to 12 membered heteroaryl are independently selected from N, O, and S.

In embodiments of a compound of Formula (V), R² is C₅₋₁₂cycloalkyl substituted with one or more substituents independently selected from R^{7′}. In embodiments of a compound of Formula (V), R² is 5 to 12 membered heterocycloalkyl substituted with one or more substituents independently selected from R^{7′}, wherein ring heteroatoms of the 5 to 12 membered heterocycloalkyl are independently selected from N, O, and S. In embodiments of a compound of Formula (V), R² is C₆₋₁₂aryl substituted with one or more substituents independently selected from R^{7′}. In embodiments of a compound of Formula (V), R² is 5 to 12 membered heteroaryl substituted with one or more substituents independently selected from R^{7′}, wherein ring heteroatoms of the 5 to 12 membered heteroaryl are independently selected from N, O, and S.

In embodiments of a compound of Formula (V), R² is C₅₋₁₂cycloalkyl substituted with one or more substituents independently selected from R⁷. In embodiments of a compound of Formula (V), R² is 5 to 12 membered heterocycloalkyl substituted with one or more substituents independently selected from R⁷, wherein ring heteroatoms of the 5 to 12 membered heterocycloalkyl are independently selected from N, O, and S. In embodiments of a compound of Formula (V), R² is C₆₋₁₂aryl substituted with one or more substituents independently selected from R⁷. In embodiments of a compound of Formula (V), R² is 5 to 12 membered heteroaryl substituted with one or more substituents independently selected from R⁷, wherein ring heteroatoms of the 5 to 12 membered heteroaryl are independently selected from N, O, and S.

In embodiments of a compound of Formula (V), R² is C₅₋₁₂cycloalkyl. In embodiments of a compound of Formula (V), R² is 5 to 12 membered heterocycloalkyl, wherein ring heteroatoms of the 5 to 12 membered heterocycloalkyl are independently selected from N, O, and S. In embodiments of a compound of Formula (V), R² is C₆₋₁₂aryl. In embodiments of a compound of Formula (V), R² is 5 to 12 membered heteroaryl, wherein ring heteroatoms of the 5 to 12 membered heteroaryl are independently selected from N, O, and S.

In embodiments of a compound of Formula (V), R^{2a} is hydrogen. In embodiments of a compound of Formula (V), R^{2a} is —OH.

In embodiments of a compound of Formula (V), R¹ is

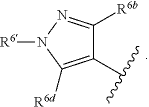

In embodiments of a compound of Formula (V), R¹ is

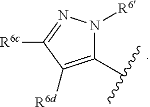

In embodiments of a compound of Formula (V), R¹ is

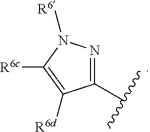

In embodiments of a compound of Formula (V), R² is

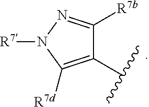

In embodiments of a compound of Formula (V), R² is

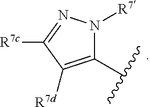

In embodiments of a compound of Formula (V), R² is

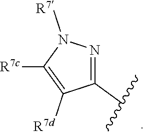

A compound of Formula (VI), or a pharmaceutically acceptable salt or solvate thereof:

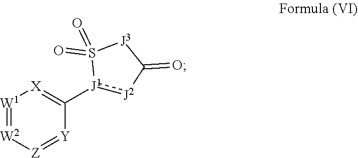

wherein:
either: W¹ is C(R¹), and W² is C(R^{2a}) or N; or W¹ is C(R^{1a}) or N, and W² is C(R²); R¹ is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are optionally substituted with one or more substituents independently selected R⁶ and R^{6′};
R^{1a} is selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20a};
R² is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are optionally substituted with one or more substituents independently selected R⁷ and R^{7′};
R^{2a} are independently selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20b};
X is N or C(R³);
Y is N or C(R⁴);
Z is N or C(R⁵);
R³ is selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)_g S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20c};
R⁴ is selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20d};
R⁵ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20e};
each R⁶ and R⁷ are independently selected from halogen, oxo, —CN, C₁₋₆alkyl, C₂₋₆alkenyl, C₂₋₆alkynyl, C₃₋₆cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁶;
each R^{6′} and R^{7′} are independently selected from —CN, C₁₋₆alkyl, C₂₋₆alkenyl, C₂₋₆alkynyl, C₃₋₆cycloalkyl, C₂₋₉heterocycloalkyl, C₆₋₁₀ aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R²⁶.
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R⁸ is selected from hydrogen, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₆cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉ heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₆cycloalkyl, C₂₋₉ heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉ heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20l};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₁₋₉heteroalkyl, C₂₋₉heterocycloalkyl, C₆₋₁₀aryl, and C₁₋₉heteroaryl are optionally substituted with one, two, or three R^{20m};
each R¹⁶ is independently selected from —C₁₋₆alkylene-OP(O)(OR^{16a})(OR⁶Ib) and —P(O)(OR^{16a})(OR^{16b}); wherein the C₁₋₆alkylene is optionally substituted with one, two, or three R²ⁿ;
each R^{16a} and R^{16b} is independently selected from hydrogen, C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₆cycloalkyl, —CH₂—C₃₋₆cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20o};
each R^{20a}, R^{20b}, R^{20c}, R^{20d}, R^{20e}, R^{20f}, R^{20h}, R²⁰ⁱ, R^{20j}, R^{20k}, R^{20l}, R^{20m}, R²⁰ⁿ, and R^{20o} are each independently selected from oxo, —CN, halogen, —CN, C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₁₋₆heteroalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl optionally substituted with one or more R²⁶, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₆cycloalkyl, C₁₋₆heteroalkyl, C₂₋₉heterocycloalkyl, C₆₋₁₀aryl, and C₁₋₉ heteroaryl;
each R²⁶ is independently selected from oxo, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three R²⁷;
each R²⁷ is independently selected from oxo, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f};
[symbol] indicates a single or double bond such that all valences are satisfied.

In embodiments of a compound of Formula (VI) described herein, or a pharmaceutically acceptable salt or solvate thereof,
R¹ is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are optionally substituted with one or more substituents independently selected R⁶ and R^{6′}; and
R² is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are optionally substituted with one or more substituents independently selected R⁷ and R^{7′}.

In some embodiments of a compound of Formula (VI), W¹ is C(R¹) and W² is C(R^{2a}). In some embodiments of a compound of Formula (VI), W¹ is C(R¹) and W² is N. In some embodiments of a compound of Formula (VI), W¹ is C(R^{1a}) and W² is C(R²). In some embodiments of a compound of Formula (VI), W¹ is N and W² is C(R²).

In some embodiments of a compound of Formula (VI), R¹ is bicyclic C₄₋₁₂cycloalkyl optionally substituted with one or more substituents independently selected from R⁶ and R^{6′}. In some embodiments of a compound of Formula (VI), R¹ is bicyclic C₂₋₁₁heterocycloalkyl optionally substituted with one or more substituents independently selected from R⁶ and R^{6′}. In some embodiments of a compound of Formula (VI), R¹ is bicyclic C₇₋₁₂aryl optionally substituted with one or more substituents independently selected from R⁶ and R^{6′}. In some embodiments of a compound of Formula (VI), R¹ is bicyclic C₁₋₁₂heteroaryl optionally substituted with one or more substituents independently selected from R⁶ and R^{6′}.

In some embodiments of a compound of Formula (VI), R¹ is bicyclic C₄₋₁₂cycloalkyl substituted with one or more substituents independently selected from R⁶ and R^{6′}. In some embodiments of a compound of Formula (VI), R¹ is bicyclic C₂₋₁₁heterocycloalkyl substituted with one or more substituents independently selected from R⁶ and R^{6′}. In some embodiments of a compound of Formula (VI), R¹ is bicyclic C₇₋₁₂aryl substituted with one or more substituents independently selected from R⁶ and R^{6′}. In some embodiments of a compound of Formula (VI), R¹ is bicyclic C₁₋₁₂heteroaryl substituted with one or more substituents independently selected from R⁶ and R^{6′}.

In some embodiments of a compound of Formula (VI), R¹ is bicyclic C₄₋₁₂cycloalkyl substituted with one or more R^{6′}. In some embodiments of a compound of Formula (VI), R¹ is bicyclic C₂₋₁₁heterocycloalkyl substituted with one or more R^{6′}. In some embodiments of a compound of Formula (VI), R¹ is bicyclic C₇₋₁₂aryl substituted with one or more R^{6′}. In some embodiments of a compound of Formula (VI), R¹ is bicyclic C₁₋₁₂heteroaryl substituted with one or more R^{6′}.

In some embodiments of a compound of Formula (VI), R¹ is bicyclic C₄₋₁₂cycloalkyl substituted with one or more R⁶. In some embodiments of a compound of Formula (VI), R¹ is bicyclic C₂₋₁₁heterocycloalkyl substituted with one or more R⁶. In some embodiments of a compound of Formula (VI), R¹ is bicyclic C₇₋₁₂aryl substituted with one or more R⁶. In some embodiments of a compound of Formula (VI), R¹ is bicyclic C₁₋₁₂heteroaryl substituted with one or more R⁶.

In some embodiments of a compound of Formula (VI), R¹ is bicyclic C₄₋₁₂cycloalkyl. In some embodiments of a compound of Formula (VI), R¹ is bicyclic C₂₋₁₁heterocycloalkyl. In some embodiments of a compound of Formula (VI), R¹ is bicyclic C₇₋₁₂aryl. In some embodiments of a compound of Formula (VI), R¹ is bicyclic C₁₋₁₂heteroaryl.

In some embodiments of a compound of Formula (VI), R² is bicyclic C₄₋₁₂cycloalkyl optionally substituted with one or more substituents independently selected from R⁷ and R^{7′}. In some embodiments of a compound of Formula (VI), R² is bicyclic C₂₋₁₁heterocycloalkyl optionally substituted with one or more substituents independently selected from R⁷ and R^{7′}. In some embodiments of a compound of Formula (VI), R² is bicyclic C₇₋₁₂aryl optionally substituted with one or more substituents independently selected from R⁷ and R^{7′}. In some embodiments of a compound of Formula (VI), R² is bicyclic C₁₋₁₂heteroaryl, optionally substituted with one or more substituents independently selected from R⁷ and R^{7′}.

In some embodiments of a compound of Formula (VI), R² is bicyclic C₄₋₁₂cycloalkyl substituted with one or more substituents independently selected from R⁷ and R^{7′}. In some embodiments of a compound of Formula (VI), R² is bicyclic C₂₋₁₁heterocycloalkyl substituted with one or more substituents independently selected from R⁷ and R^{7′}. In some embodiments of a compound of Formula (VI), R² is bicyclic C₇₋₁₂aryl substituted with one or more substituents independently selected from R⁷ and R^{7′}. In some embodiments of a compound of Formula (VI), R² is bicyclic C₁₋₁₂heteroaryl, substituted with one or more substituents independently selected from R⁷ and R^{7′}.

In some embodiments of a compound of Formula (VI), R² is bicyclic C₄₋₁₂cycloalkyl substituted with one or more R^{7′}. In some embodiments of a compound of Formula (VI), R² is bicyclic C₂₋₁₁heterocycloalkyl substituted with one or more R^{7′}. In some embodiments of a compound of Formula (VI), R² is bicyclic C₇₋₁₂aryl substituted with one or more R^{7′}. In some embodiments of a compound of Formula (VI), R² is bicyclic C₁₋₁₂heteroaryl, substituted with one or more R^{7′}.

In some embodiments of a compound of Formula (VI), R² is bicyclic C₄₋₁₂cycloalkyl substituted with one or more R⁷. In some embodiments of a compound of Formula (VI), R² is bicyclic C₂₋₁₁heterocycloalkyl substituted with one or more R⁷. In some embodiments of a compound of Formula (VI), R² is bicyclic C₇₋₁₂aryl substituted with one or more R⁷. In some embodiments of a compound of Formula (VI), R² is bicyclic C₁₋₁₂heteroaryl, substituted with one or more R⁷.

In some embodiments of a compound of Formula (VI), R² is bicyclic C₄₋₁₂cycloalkyl. In some embodiments of a compound of Formula (VI), R² is bicyclic C₂₋₁₁heterocycloalkyl. In some embodiments of a compound of Formula (VI), R² is bicyclic C₇₋₁₂aryl. In some embodiments of a compound of Formula (VI), R² is bicyclic C₁₋₁₂heteroaryl.

In some embodiments of a compound of Formula (VI), R^{1a} is hydrogen.

In some embodiments of a compound of Formula (VI), R^{2a} is hydrogen.

In an aspect is provided a compound of Formula (VII), or a pharmaceutically acceptable salt or solvate thereof:

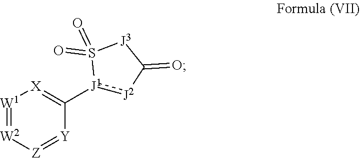

wherein:
either:
W¹ is C(R¹), and W² is C(R^{2a}) or N; or
W¹ is C(R^{1a}) or N, and W² is C(R²);
R¹ is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are optionally substituted with one or more substituents independently selected from R⁶ and R^{6′}, wherein ring heteroatoms of the 5 to 12 membered heterocycloalkyl and 5 to 12 membered heteroaryl are independently selected from N, O, and S;
R^{1a} is selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20a};
R² is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are optionally substituted with one or more substituents independently selected from R⁷ and R^{7′}, wherein ring heteroatoms of the 5 to 12 membered heterocycloalkyl and 5 to 12 membered heteroaryl are independently selected from N, O, and S;
R^{2a} are independently selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20b};
X is N or C(R³);
Y is N or C(R⁴);
Z is N or C(R⁵);
R³ is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R⁴ is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20e};
each R⁶ and R⁷ are independently selected from halogen, oxo, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁶;
each R^{6′} and R^{7′} are independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀ aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶;
J¹ is N, C, or C(R⁸);
J² is N, N(R⁹), C(R⁹), C(R⁹)(R^{9a}), or C(O);
J³ is N(R¹⁰) or C(R¹⁰)(R^{10a});
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉ heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉ heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20l};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted with one, two, or three R^{20m};
each R¹⁶ is independently selected from —C₁₋₆alkylene-OP(O)(OR^{16a})(OR⁶Ib) and —P(O)(OR^{16a})(OR⁶Ib); wherein the C₁₋₆alkylene is optionally substituted with one, two, or three R²⁰ⁿ; or —C₁₋₆alkylene-OC(O)—R^{16c} wherein C₁₋₆alkylene is optionally substituted with one, two, or three R²⁰ⁿ;
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20o};
R^{16c} is halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —CH₂—C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three of R²⁰;
each R^{20a}, R^{20b}, R^{20c}, R^{20d}, R^{20e}, R^{20f}, R^{20h}, R²⁰ⁱ, R^{20j}, R^{20k}, R^{20l}, R^{20m}, R²⁰ⁿ, and R^{20o} are each independently selected from oxo, —CN, halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl optionally substituted with one or more R²⁶, C₁₋₆haloalkyl, C₁₋₆alkoxy, C₁₋₉haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉ heteroaryl;
each R²⁶ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁷;
each R²⁷ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20f};
[symbol] indicates a single or double bond such that all valences are satisfied.

In embodiments of a compound of Formula (VII) described herein, or a pharmaceutically acceptable salt or solvate thereof,
R¹ is selected from C₅₋₁₂cycloalkyl, 5 to 12 membered heterocycloalkyl, C₇₋₁₂aryl, and 5 to 12 membered heteroaryl, wherein C₅₋₁₂cycloalkyl, 5 to 12 membered heterocycloalkyl, C₇₋₁₂aryl, and 5 to 12 membered heteroaryl are optionally substituted with one or more substituents independently selected from R⁶ and R^{6′}, wherein ring heteroatoms of the 5 to 12 membered heterocycloalkyl and 5 to 12 membered heteroaryl are independently selected from C, N, O, and S; and
R² is selected from C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl, wherein C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl are optionally substituted with one or more substituents independently selected from R⁷ and R^{7′}, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S.

In embodiments of a compound of Formula (VII) described herein, or a pharmaceutically acceptable salt or solvate thereof,
R¹ is selected from triazolyl, pyrazolyl, pyrrolyl, imidazolyl, pyridyl, pyrimidinyl, pyridazinyl, pyrazinyl, and triazinyl, wherein triazolyl, pyrazolyl, pyrrolyl, imidazolyl, phenyl, pyridyl, pyrimidinyl, pyridazinyl, pyrazinyl, and triazinyl are optionally substituted with one or more substituents independently selected from R⁶ and R^{6′}; and
R² is selected from triazolyl, pyrazolyl, pyrrolyl, imidazolyl, pyridyl, pyrimidinyl, pyridazinyl, pyrazinyl, and triazinyl, wherein triazolyl, pyrazolyl, pyrrolyl, imidazolyl, phenyl, pyridyl, pyrimidinyl, pyridazinyl, pyrazinyl, and triazinyl are optionally substituted with one or more substituents independently selected from R⁷ and R^{7′}.

In embodiments of a compound of Formula (VII) described herein, or a pharmaceutically acceptable salt or solvate thereof,
R¹ is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are optionally substituted with one or more substituents independently selected from R⁶ and R^{6′};
R² is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are optionally substituted with one or more substituents independently selected from R⁷ and R^{7′}.

In embodiments of a compound of Formula (VII) described herein, or a pharmaceutically acceptable salt or solvate thereof,
R¹ is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are optionally substituted with one or more substituents independently selected from R⁶ and R^{6′}; and
R² is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are optionally substituted with one or more substituents independently selected from R⁷ and R^{7′}.

In embodiments of a compound of Formula (VII) described herein, or a pharmaceutically acceptable salt or solvate thereof, R³ is Cl; R¹ is pyrazolyl optionally substituted with one or more substituents independently selected from R⁶ and R^{6′}; and R² is pyrazolyl optionally substituted with one or more substituents independently selected from R⁷ and R^{7′}.

In embodiments of a compound of Formula (VII) described herein, or a pharmaceutically acceptable salt or solvate thereof, R³ is Cl; R¹ is pyrazolyl substituted with one or more substituents independently selected from R⁶ and R^{6′}; and R² is pyrazolyl substituted with one or more substituents independently selected from R⁷ and R^{7′}.

In embodiments of a compound of Formula (VII) described herein, or a pharmaceutically acceptable salt or solvate thereof, R³ is Cl; R¹ is pyrazolyl optionally substituted with one or more substituents independently selected from R^{6′}; and R² is pyrazolyl optionally substituted with one or more substituents independently selected from R^{7′}.

In embodiments of a compound of Formula (VII) described herein, or a pharmaceutically acceptable salt or solvate thereof, R³ is Cl; R¹ is pyrazolyl substituted with one or more substituents independently selected from R^{6′}; and R² is pyrazolyl substituted with one or more substituents independently selected from R^{7′}.

In embodiments of a compound of Formula (VII) described herein, or a pharmaceutically acceptable salt or solvate thereof, R³ is Cl; R¹ is pyrazolyl optionally substituted with one R^{6′}; and R² is pyrazolyl optionally substituted with one R^{7′}.

In embodiments of a compound of Formula (VII) described herein, or a pharmaceutically acceptable salt or solvate thereof, R³ is Cl; R¹ is pyrazolyl substituted with one R^{6′}; and R² is pyrazolyl substituted with one R^{7′}.

In embodiments, W¹ is C(R¹), and W² is C(R^{2a}). In embodiments, W¹ is C(R¹), and W² is N. In embodiments, W¹ is C(R^{1a}) and W² is C(R²). In embodiments, W¹ is N and W² is C(R²).

In embodiments of a compound of Formula (VII), W¹ is C(R¹) and W² is C(R^{2a}). In embodiments of a compound of Formula (VII), W¹ is C(R^{1a}) and W² is C(R²). In embodiments of a compound of Formula (VII), W¹ is C(R¹) and W² is N. In embodiments of a compound of Formula (VII), W¹ is N and W² is C(R²).

In embodiments of a compound of Formula (VII), R¹ is C₅₋₁₂cycloalkyl optionally substituted with one or more substituents independently selected from R⁶ and R^{6′}. In embodiments of a compound of Formula (VII), R¹ is 5 to 12 membered heterocycloalkyl optionally substituted with one or more substituents independently selected from R⁶ and R^{6′}, wherein ring heteroatoms of the 5 to 12 membered heterocycloalkyl are independently selected from N, O, and S. In embodiments of a compound of Formula (VII), R¹ is C₆₋₁₂aryl optionally substituted with one or more substituents independently selected from R⁶ and R^{6′}. In embodiments of a compound of Formula (VII), R¹ is 5 to 12 membered heteroaryl optionally substituted with one or more substituents independently selected from R⁶ and R^{6′}, wherein ring heteroatoms of the 5 to 12 membered heteroaryl are independently selected from N, O, and S.

In embodiments of a compound of Formula (VII), R¹ is C₅₋₁₂cycloalkyl substituted with one or more substituents independently selected from R⁶ and R^{6′}. In embodiments of a compound of Formula (VII), R¹ is 5 to 12 membered heterocycloalkyl substituted with one or more substituents independently selected from R⁶ and R^{6′}, wherein ring heteroatoms of the 5 to 12 membered heterocycloalkyl are independently selected from N, O, and S. In embodiments of a compound of Formula (VII), R¹ is C₆₋₁₂aryl substituted with one or more substituents independently selected from R⁶ and R^{6′}. In embodiments of a compound of Formula (VII),
R¹ is 5 to 12 membered heteroaryl substituted with one or more substituents independently selected from R⁶ and R^{6′}, wherein ring heteroatoms of the 5 to 12 membered heteroaryl are independently selected from N, O, and S.

In embodiments of a compound of Formula (VII), R¹ is C₅₋₁₂cycloalkyl substituted with one or more substituents independently selected from R^{6′}. In embodiments of a compound of Formula (VII), R¹ is 5 to 12 membered heterocycloalkyl substituted with one or more substituents independently selected from R^{6′}, wherein ring heteroatoms of the 5 to 12 membered heterocycloalkyl are independently selected from N, O, and S. In embodiments of a compound of Formula (VII), R¹ is C₆₋₁₂aryl substituted with one or more substituents independently selected from R^{6′}. In embodiments of a compound of Formula (VII), R¹ is 5 to 12 membered heteroaryl substituted with one or more substituents independently selected from R^{6′}, wherein ring heteroatoms of the 5 to 12 membered heteroaryl are independently selected from N, O, and S.

In embodiments of a compound of Formula (VII), R¹ is C₅₋₁₂cycloalkyl substituted with one or more substituents independently selected from R⁶. In embodiments of a compound of Formula (VII), R¹ is 5 to 12 membered heterocycloalkyl substituted with one or more substituents independently selected from R⁶, wherein ring heteroatoms of the 5 to 12 membered heterocycloalkyl are independently selected from N, O, and S. In embodiments of a compound of Formula (VII), R¹ is C₆₋₁₂aryl substituted with one or more substituents independently selected from R⁶. In embodiments of a compound of Formula (VII), R¹ is 5 to 12 membered heteroaryl substituted with one or more substituents independently selected from R⁶, wherein ring heteroatoms of the 5 to 12 membered heteroaryl are independently selected from N, O, and S.

In embodiments of a compound of Formula (VII), R¹ is C₅₋₁₂cycloalkyl. In embodiments of a compound of Formula (VII), R¹ is 5 to 12 membered heterocycloalkyl, wherein ring heteroatoms of the 5 to 12 membered heterocycloalkyl are independently selected from N, O, and S. In embodiments of a compound of Formula (VII), R¹ is C₆₋₁₂aryl. In embodiments of a compound of Formula (VII), R¹ is 5 to 12 membered heteroaryl, wherein ring heteroatoms of the 5 to 12 membered heteroaryl are independently selected from N, O, and S.

In embodiments of a compound of Formula (VII), R^{1a} is hydrogen.

In embodiments of a compound of Formula (VII), R² is C₅₋₁₂cycloalkyl optionally substituted with one or more substituents independently selected from R⁷ and R^{7′}. In embodiments of a compound of Formula (VII), R² is 5 to 12 membered heterocycloalkyl optionally substituted with one or more substituents independently selected from R⁷ and R^{7′} wherein ring heteroatoms of the 5 to 12 membered heterocycloalkyl are independently selected from N, O, and S. In embodiments of a compound of Formula (VII), R² is C₆₋₁₂aryl optionally substituted with one or more substituents independently selected from R⁷ and R^{7′}. In embodiments of a compound of Formula (VII), R² is 5 to 12 membered heteroaryl optionally substituted with one or more substituents independently selected from R⁷ and R^{7′} wherein ring heteroatoms of the 5 to 12 membered heteroaryl are independently selected from N, O, and S.

In embodiments of a compound of Formula (VII), R² is C₅₋₁₂cycloalkyl substituted with one or more substituents independently selected from R⁷ and R^{7′}. In embodiments of a compound of Formula (VII), R² is 5 to 12 membered heterocycloalkyl substituted with one or more substituents independently selected from R⁷ and R^{7′}, wherein ring heteroatoms of the 5 to 12 membered heterocycloalkyl are independently selected from N, O, and S. In embodiments of a compound of Formula (VII), R² is C₆₋₁₂aryl substituted with one or more substituents independently selected from R⁷ and R^{7′}. In embodiments of a compound of Formula (VII), R² is 5 to 12 membered heteroaryl substituted with one or more substituents independently selected from R⁷ and R^{7′}, wherein ring heteroatoms of the 5 to 12 membered heteroaryl are independently selected from N, O, and S.

In embodiments of a compound of Formula (VII), R² is C₅₋₁₂cycloalkyl substituted with one or more substituents independently selected from R^{7′}. In embodiments of a compound of Formula (VII), R² is 5 to 12 membered heterocycloalkyl substituted with one or more substituents independently selected from R^{7′}, wherein ring heteroatoms of the 5 to 12 membered heterocycloalkyl are independently selected from N, O, and S. In embodiments of a compound of Formula (VII), R² is C₆₋₁₂aryl substituted with one or more substituents independently selected from R^{7′}. In embodiments of a compound of Formula (VII), R² is 5 to 12 membered heteroaryl substituted with one or more substituents independently selected from R^{7′}, wherein ring heteroatoms of the 5 to 12 membered heteroaryl are independently selected from N, O, and S.

In embodiments of a compound of Formula (VII), R² is C₅₋₁₂cycloalkyl substituted with one or more substituents independently selected from R⁷. In embodiments of a compound of Formula (VII), R² is 5 to 12 membered heterocycloalkyl substituted with one or more substituents independently selected from R⁷, wherein ring heteroatoms of the 5 to 12 membered heterocycloalkyl are independently selected from N, O, and S. In embodiments of a compound of Formula (VII), R² is C₆₋₁₂aryl substituted with one or more substituents independently selected from R⁷. In embodiments of a compound of Formula (VII), R² is 5 to 12 membered heteroaryl substituted with one or more substituents independently selected from R⁷, wherein ring heteroatoms of the 5 to 12 membered heteroaryl are independently selected from N, O, and S.

In embodiments of a compound of Formula (VII), R² is C₅₋₁₂cycloalkyl. In embodiments of a compound of Formula (VII), R² is 5 to 12 membered heterocycloalkyl, wherein ring heteroatoms of the 5 to 12 membered heterocycloalkyl are independently selected from N, O, and S. In embodiments of a compound of Formula (VII), R² is C₆₋₁₂aryl. In embodiments of a compound of Formula (VII), R² is 5 to 12 membered heteroaryl, wherein ring heteroatoms of the 5 to 12 membered heteroaryl are independently selected from N, O, and S.

In embodiments of a compound of Formula (VII), R³ is selected from halogen and C₁₋₆alkyl, wherein C₁₋₆alkyl, is optionally substituted with one, two, or three R^{20c}. In embodiments of a compound of Formula (VII), R³ is halogen.

In embodiments of a compound of Formula (VII), R⁴ is selected from —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³). In embodiments of a compound of Formula (VII), R⁴ is —OR¹². In embodiments of a compound of Formula (VII), R⁴ is —OH.

In embodiments of a compound of Formula (VII), R⁵ is hydrogen. In embodiments of a compound of Formula (VII), R⁵ is —OH.

In embodiments of a compound of Formula (VII), R¹ is selected from C₅₋₁₂cycloalkyl, 5 to 12 membered heterocycloalkyl, C₇₋₁₂aryl, and 5 to 12 membered heteroaryl, wherein C₅₋₁₂cycloalkyl, 5 to 12 membered heterocycloalkyl, C₇₋₁₂aryl, and 5 to 12 membered heteroaryl are optionally substituted with one or more substituents independently selected from R⁶ and R^{6′}, wherein ring heteroatoms of the 5 to 12 membered heterocycloalkyl and 5 to 12 membered heteroaryl are independently selected from C, N, O, and S. In embodiments of a compound of Formula (VII), R¹ is C₇₋₁₂aryl optionally substituted with one or more substituents independently selected from R⁶ and R^{6′}. In embodiments of a compound of Formula (VII), R¹ is C₇₋₁₂aryl substituted with one or more substituents independently selected from R⁶ and R^{6′}. In embodiments of a compound of Formula (VII), R¹ is C₇₋₁₂aryl substituted with one or more substituents independently selected from R^{6′}. In embodiments of a compound of Formula (VII), R¹ is C₇₋₁₂aryl substituted with one or more substituents independently selected from R⁶. In embodiments of a compound of Formula (VII), R¹ is C₇₋₁₂aryl.

In embodiments of a compound of Formula (VII), R² is selected from C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl, wherein C₅₋₁₂cycloalkyl, 5 to 12 membered partially unsaturated heterocycloalkyl, 6 to 12 membered saturated heterocycloalkyl, C₇₋₁₂aryl, and 5 to 12 membered heteroaryl are optionally substituted with one or more substituents independently selected from R⁷ and R^{7′}, wherein ring heteroatoms of the 5 to 12 membered partially unsaturated heterocycloalkyl, 6 to 12 membered saturated heterocycloalkyl, and 5 to 12 membered heteroaryl are independently selected from C, N, O, and S.

In embodiments of a compound of Formula (VII), R² is 5 to 12 membered partially unsaturated heterocycloalkyl optionally substituted with one or more substituents independently selected from R⁷ and R^{7′}, wherein ring heteroatoms of the 5 to 12 membered partially unsaturated heterocycloalkyl are independently selected from C, N, O, and S. In embodiments of a compound of Formula (VII), R² is 5 to 12 membered partially unsaturated heterocycloalkyl substituted with one or more substituents independently selected from R⁷ and R^{7′}, wherein ring heteroatoms of the 5 to 12 membered partially unsaturated heterocycloalkyl are independently selected from C, N, O, and S. In embodiments of a compound of Formula (VII), R² is 5 to 12 membered partially unsaturated heterocycloalkyl substituted with one or more substituents independently selected from R^{7′}, wherein ring heteroatoms of the 5 to 12 membered partially unsaturated heterocycloalkyl are independently selected from C, N, O, and S. In embodiments of a compound of Formula (VII), R² is 5 to 12 membered partially unsaturated heterocycloalkyl substituted with one or more substituents independently selected from R⁷, wherein ring heteroatoms of the 5 to 12 membered partially unsaturated heterocycloalkyl are independently selected from C, N, O, and S. In embodiments of a compound of Formula (VII), R² is 5 to 12 membered partially unsaturated heterocycloalkyl, wherein ring heteroatoms of the 5 to 12 membered partially unsaturated heterocycloalkyl are independently selected from C, N, O, and S.

In embodiments of a compound of Formula (VII), R² is 6 to 12 membered saturated heterocycloalkyl optionally substituted with one or more substituents independently selected from R⁷ and R^{7′}, wherein ring heteroatoms of the 6 to 12 membered saturated heterocycloalkyl are independently selected from C, N, O, and S. In embodiments of a compound of Formula (VII), R² is 6 to 12 membered saturated heterocycloalkyl substituted with one or more substituents independently selected from R⁷ and R^{7′}, wherein ring heteroatoms of the 6 to 12 membered saturated heterocycloalkyl are independently selected from C, N, O, and S. In embodiments of a compound of Formula (VII), R² is 6 to 12 membered saturated heterocycloalkyl substituted with one or more substituents independently selected from R^{7′}, wherein ring heteroatoms of the 6 to 12 membered saturated heterocycloalkyl are independently selected from C, N, O, and S. In embodiments of a compound of Formula (VII), R² is 6 to 12 membered saturated heterocycloalkyl substituted with one or more substituents independently selected from R⁷, wherein ring heteroatoms of the 6 to 12 membered saturated heterocycloalkyl are independently selected from C, N, O, and S. In embodiments of a compound of Formula (VII), R² is 6 to 12 membered saturated heterocycloalkyl, wherein ring heteroatoms of the 6 to 12 membered saturated heterocycloalkyl are independently selected from C, N, O, and S.

In embodiments of a compound of Formula (VII), R² is C₇₋₁₂aryl optionally substituted with one or more substituents independently selected from R⁷ and R^{7′}. In embodiments of a compound of Formula (VII), R² is C₇₋₁₂aryl substituted with one or more substituents independently selected from R⁷ and R^{7′}. In embodiments of a compound of Formula (VII), R² is C₇₋₁₂aryl substituted with one or more substituents independently selected from R^{7′}. In embodiments of a compound of Formula (VII), R² is C₇₋₁₂aryl substituted with one or more substituents independently selected from R⁷. In embodiments of a compound of Formula (VII), R² is C₇₋₁₂aryl.

In embodiments of a compound of Formula (VII), R¹ is selected from triazolyl, pyrazolyl, pyrrolyl, imidazolyl, pyridyl, pyrimidinyl, pyridazinyl, pyrazinyl, and triazinyl, wherein triazolyl, pyrazolyl, pyrrolyl, imidazolyl, phenyl, pyridyl, pyrimidinyl, pyridazinyl, pyrazinyl, and triazinyl are optionally substituted with one or more substituents independently selected from R⁶ and R^{6′}. In embodiments of a compound of Formula (VII), R¹ is selected from triazolyl, pyrazolyl, pyrrolyl, imidazolyl, pyridyl, pyrimidinyl, pyridazinyl, pyrazinyl, and triazinyl, wherein triazolyl, pyrazolyl, pyrrolyl, imidazolyl, phenyl, pyridyl, pyrimidinyl, pyridazinyl, pyrazinyl, and triazinyl are optionally substituted with one or more substituents independently selected from R^{6′}. In embodiments of a compound of Formula (VII), R¹ is selected from triazolyl, pyrazolyl, pyrrolyl, imidazolyl, pyridyl, pyrimidinyl, pyridazinyl, pyrazinyl, and triazinyl, wherein triazolyl, pyrazolyl, pyrrolyl, imidazolyl, phenyl, pyridyl, pyrimidinyl, pyridazinyl, pyrazinyl, and triazinyl are optionally substituted with one or more substituents independently selected from R⁶.

In embodiments of a compound of Formula (VII), R² is selected from triazolyl, pyrazolyl, pyrrolyl, imidazolyl, pyridyl, pyrimidinyl, pyridazinyl, pyrazinyl, and triazinyl, wherein triazolyl, pyrazolyl, pyrrolyl, imidazolyl, phenyl, pyridyl, pyrimidinyl, pyridazinyl, pyrazinyl, and triazinyl are optionally substituted with one or more substituents independently selected from R⁷ and R^{7′}. In embodiments of a compound of Formula (VII), R² is selected from triazolyl, pyrazolyl, pyrrolyl, imidazolyl, pyridyl, pyrimidinyl, pyridazinyl, pyrazinyl, and triazinyl, wherein triazolyl, pyrazolyl, pyrrolyl, imidazolyl, phenyl, pyridyl, pyrimidinyl, pyridazinyl, pyrazinyl, and triazinyl are optionally substituted with one or more substituents independently selected from R^{7′}. In embodiments of a compound of Formula (VII), R² is selected from triazolyl, pyrazolyl, pyrrolyl, imidazolyl, pyridyl, pyrimidinyl, pyridazinyl, pyrazinyl, and triazinyl, wherein triazolyl, pyrazolyl, pyrrolyl, imidazolyl, phenyl, pyridyl, pyrimidinyl, pyridazinyl, pyrazinyl, and triazinyl are optionally substituted with one or more substituents independently selected from R⁷.

In embodiments of a compound of Formula (VII), R¹ is triazolyl substituted with one or more substituents independently selected from R⁶ and R^{6′}. In embodiments of a compound of Formula (VII), R¹ is pyrazolyl substituted with one or more substituents independently selected from R⁶ and R^{6′}. In embodiments of a compound of Formula (VII), R¹ is pyrrolyl substituted with one or more substituents independently selected from R⁶ and R^{6′}. In embodiments of a compound of Formula (VII), R¹ is imidazolyl substituted with one or more substituents independently selected from R⁶ and R^{6′}. In embodiments of a compound of Formula (VII), R¹ is phenyl substituted with one or more substituents independently selected from R⁶ and R^{6′}. In embodiments of a compound of Formula (VII), R¹ is pyridyl substituted with one or more substituents independently selected from R⁶ and R^{6′}. In embodiments of a compound of Formula (VII), R¹ is pyrimidinyl substituted with one or more substituents independently selected from R⁶ and R^{6′}. In embodiments of a compound of Formula (VII), R¹ is pyridazinyl substituted with one or more substituents independently selected from R⁶ and R^{6′}. In embodiments of a compound of Formula (VII), R¹ is pyrazinyl substituted with one or more substituents independently selected from R⁶ and R^{6′}. In embodiments of a compound of Formula (VII), R¹ is triazinyl substituted with one or more substituents independently selected from R⁶ and R^{6′}. In embodiments of a compound of Formula (VII), R¹ is furanyl substituted with one or more substituents independently selected from R⁶ and R^{6′}. In embodiments of a compound of Formula (VII), R¹ is thienyl substituted with one or more substituents independently selected from R⁶ and R^{6′}. In embodiments of a compound of Formula (VII), R¹ is oxazolyl substituted with one or more substituents independently selected from R⁶ and R^{6′}. In embodiments of a compound of Formula (VII), R¹ is isoxazolyl substituted with one or more substituents independently selected from R⁶ and R^{6′}. In embodiments of a compound of Formula (VII), R¹ is thiazolyl substituted with one or more substituents independently selected from R⁶ and R^{6′}. In embodiments of a compound of Formula (VII), R¹ is isothiazolyl substituted with one or more substituents independently selected from R⁶ and R^{6′}. In embodiments of a compound of Formula (VII), R¹ is oxadiazolyl substituted with one or more substituents independently selected from R⁶ and R^{6′}. In embodiments of a compound of Formula (VII), R¹ is thiadiazolyl substituted with one or more substituents independently selected from R⁶ and R^{6′}.

In embodiments of a compound of Formula (VII), R¹ is triazolyl substituted with one or more substituents independently selected from R^{6′}. In embodiments of a compound of Formula (VII), R¹ is pyrazolyl substituted with one or more substituents independently selected from R^{6′}. In embodiments of a compound of Formula (VII), R¹ is pyrrolyl substituted with one or more substituents independently selected from R^{6′}. In embodiments of a compound of Formula (VII), R¹ is imidazolyl substituted with one or more substituents independently selected from R^{6′}. In embodiments of a compound of Formula (VII), R¹ is phenyl substituted with one or more substituents independently selected from R^{6′}. In embodiments of a compound of Formula (VII), R¹ is pyridyl substituted with one or more substituents independently selected from R^{6′}. In embodiments of a compound of Formula (VII), R¹ is pyrimidinyl substituted with one or more substituents independently selected from R^{6′}. In embodiments of a compound of Formula (VII), R¹ is pyridazinyl substituted with one or more substituents independently selected from R^{6′}. In embodiments of a compound of Formula (VII), R¹ is pyrazinyl substituted with one or more substituents independently selected from R^{6′}. In embodiments of a compound of Formula (VII), R¹ is triazinyl substituted with one or more substituents independently selected from R^{6′}. In embodiments of a compound of Formula (VII), R¹ is furanyl substituted with one or more substituents independently selected from R^{6′}. In embodiments of a compound of Formula (VII), R¹ is thienyl substituted with one or more substituents independently selected from R^{6′}. In embodiments of a compound of Formula (VII), R¹ is oxazolyl substituted with one or more substituents independently selected from R^{6′}. In embodiments of a compound of Formula (VII), R¹ is isoxazolyl substituted with one or more substituents independently selected from R^{6′}. In embodiments of a compound of Formula (VII), R¹ is thiazolyl substituted with one or more substituents independently selected from R^{6′}. In embodiments of a compound of Formula (VII), R¹ is isothiazolyl substituted with one or more substituents independently selected from R^{6′}. In embodiments of a compound of Formula (VII), R¹ is oxadiazolyl substituted with one or more substituents independently selected from R^{6′}. In embodiments of a compound of Formula (VII), R¹ is thiadiazolyl substituted with one or more substituents independently selected from R^{6′}.

In embodiments of a compound of Formula (VII), R¹ is triazolyl substituted with one or more substituents independently selected from R⁶. In embodiments of a compound of Formula (VII), R¹ is pyrazolyl substituted with one or more substituents independently selected from R⁶. In embodiments of a compound of Formula (VII), R¹ is pyrrolyl substituted with one or more substituents independently selected from R⁶. In embodiments of a compound of Formula (VII), R¹ is imidazolyl substituted with one or more substituents independently selected from R⁶. In embodiments of a compound of Formula (VII), R¹ is phenyl substituted with one or more substituents independently selected from R⁶. In embodiments of a compound of Formula (VII), R¹ is pyridyl substituted with one or more substituents independently selected from R⁶. In embodiments of a compound of Formula (VII), R¹ is pyrimidinyl substituted with one or more substituents independently selected from R⁶. In embodiments of a compound of Formula (VII), R¹ is pyridazinyl substituted with one or more substituents independently selected from R⁶. In embodiments of a compound of Formula (VII), R¹ is pyrazinyl substituted with one or more substituents independently selected from R⁶. In embodiments of a compound of Formula (VII), R¹ is triazinyl substituted with one or more substituents independently selected from R⁶. In embodiments of a compound of Formula (VII), R¹ is furanyl substituted with one or more substituents independently selected from R⁶. In embodiments of a compound of Formula (VII), R¹ is thienyl substituted with one or more substituents independently selected from R⁶. In embodiments of a compound of Formula (VII), R¹ is oxazolyl substituted with one or more substituents independently selected from R⁶. In embodiments of a compound of Formula (VII), R¹ is isoxazolyl substituted with one or more substituents independently selected from R⁶. In embodiments of a compound of Formula (VII), R¹ is thiazolyl substituted with one or more substituents independently selected from R⁶. In embodiments of a compound of Formula (VII), R¹ is isothiazolyl substituted with one or more substituents independently selected from R⁶. In embodiments of a compound of Formula (VII), R¹ is oxadiazolyl substituted with one or more substituents independently selected from R⁶. In embodiments of a compound of Formula (VII), R¹ is thiadiazolyl substituted with one or more substituents independently selected from R⁶.

In embodiments of a compound of Formula (VII), R² is triazolyl substituted with one or more substituents independently selected from R⁷ and R^{7′}. In embodiments of a compound of Formula (VII), R² is pyrazolyl substituted with one or more substituents independently selected from R⁷ and R^{7′}. In embodiments of a compound of Formula (VII), R² is pyrrolyl substituted with one or more substituents independently selected from R⁷ and R^{7′}. In embodiments of a compound of Formula (VII), R² is imidazolyl substituted with one or more substituents independently selected from R⁷ and R^{7′}. In embodiments of a compound of Formula (VII), R² is phenyl substituted with one or more substituents independently selected from R⁷ and R^{7′}. In embodiments of a compound of Formula (VII), R² is pyridyl substituted with one or more substituents independently selected from R⁷ and R^{7′}. In embodiments of a compound of Formula (VII), R² is pyrimidinyl substituted with one or more substituents independently selected from R⁷ and R^{7′}. In embodiments of a compound of Formula (VII), R² is pyridazinyl substituted with one or more substituents independently selected from R⁷ and R^{7′}. In embodiments of a compound of Formula (VII), R² is pyrazinyl substituted with one or more substituents independently selected from R⁷ and R^{7′}. In embodiments of a compound of Formula (VII), R² is triazinyl substituted with one or more substituents independently selected from R⁷ and R^{7′}. In embodiments of a compound of Formula (VII), R² is furanyl substituted with one or more substituents independently selected from R⁷ and R^{7′}. In embodiments of a compound of Formula (VII), R² is thienyl substituted with one or more substituents independently selected from R⁷ and R^{7′}. In embodiments of a compound of Formula (VII), R² is oxazolyl substituted with one or more substituents independently selected from R⁷ and R^{7′}. In embodiments of a compound of Formula (VII), R² is isoxazolyl substituted with one or more substituents independently selected from R⁷ and R^{7′}. In embodiments of a compound of Formula (VII), R² is thiazolyl substituted with one or more substituents independently selected from R⁷ and R^{7′}. In embodiments of a compound of Formula (VII), R² is isothiazolyl substituted with one or more substituents independently selected from R⁷ and R^{7′}. In embodiments of a compound of Formula (VII), R² is oxadiazolyl substituted with one or more substituents independently selected from R⁷ and R^{7′}. In embodiments of a compound of Formula (VII), R² is thiadiazolyl substituted with one or more substituents independently selected from R⁷ and R^{7′}.

In embodiments of a compound of Formula (VII), R² is triazolyl substituted with one or more substituents independently selected from R^{7′}. In embodiments of a compound of Formula (VII), R² is pyrazolyl substituted with one or more substituents independently selected from R^{7′}. In embodiments of a compound of Formula (VII), R² is pyrrolyl substituted with one or more substituents independently selected from R^{7′}. In embodiments of a compound of Formula (VII), R² is imidazolyl substituted with one or more substituents independently selected from R^{7′}. In embodiments of a compound of Formula (VII), R² is phenyl substituted with one or more substituents independently selected from R^{7′}. In embodiments of a compound of Formula (VII), R² is pyridyl substituted with one or more substituents independently selected from R^{7′}. In embodiments of a compound of Formula (VII), R² is pyrimidinyl substituted with one or more substituents independently selected from R^{7′}. In embodiments of a compound of Formula (VII), R² is pyridazinyl substituted with one or more substituents independently selected from R^{7′}. In embodiments of a compound of Formula (VII), R² is pyrazinyl substituted with one or more substituents independently selected from R^{7′}. In embodiments of a compound of Formula (VII), R² is triazinyl substituted with one or more substituents independently selected from R^{7′}. In embodiments of a compound of Formula (VII), R² is furanyl substituted with one or more substituents independently selected from R^{7′}. In embodiments of a compound of Formula (VII), R² is thienyl substituted with one or more substituents independently selected from R^{7′}. In embodiments of a compound of Formula (VII), R² is oxazolyl substituted with one or more substituents independently selected from R^{7′}. In embodiments of a compound of Formula (VII), R² is isoxazolyl substituted with one or more substituents independently selected from R^{7′}. In embodiments of a compound of Formula (VII), R² is thiazolyl substituted with one or more substituents independently selected from R^{7′}. In embodiments of a compound of Formula (VII), R² is isothiazolyl substituted with one or more substituents independently selected from R^{7′}. In embodiments of a compound of Formula (VII), R² is oxadiazolyl substituted with one or more substituents independently selected from R^{7′}. In embodiments of a compound of Formula (VII), R² is thiadiazolyl substituted with one or more substituents independently selected from R^{7′}.

In embodiments of a compound of Formula (VII), R² is triazolyl substituted with one or more substituents independently selected from R⁷. In embodiments of a compound of Formula (VII), R² is pyrazolyl substituted with one or more substituents independently selected from R⁷. In embodiments of a compound of Formula (VII), R² is pyrrolyl substituted with one or more substituents independently selected from R⁷. In embodiments of a compound of Formula (VII), R² is imidazolyl substituted with one or more substituents independently selected from R⁷. In embodiments of a compound of Formula (VII), R² is phenyl substituted with one or more substituents independently selected from R⁷. In embodiments of a compound of Formula (VII), R² is pyridyl substituted with one or more substituents independently selected from R⁷. In embodiments of a compound of Formula (VII), R² is pyrimidinyl substituted with one or more substituents independently selected from R⁷. In embodiments of a compound of Formula (VII), R² is pyridazinyl substituted with one or more substituents independently selected from R⁷. In embodiments of a compound of Formula (VII), R² is pyrazinyl substituted with one or more substituents independently selected from R⁷. In embodiments of a compound of Formula (VII), R² is triazinyl substituted with one or more substituents independently selected from R⁷. In embodiments of a compound of Formula (VII), R² is furanyl substituted with one or more substituents independently selected from R⁷. In embodiments of a compound of Formula (VII), R² is thienyl substituted with one or more substituents independently selected from R⁷. In embodiments of a compound of Formula (VII), R² is oxazolyl substituted with one or more substituents independently selected from R⁷. In embodiments of a compound of Formula (VII), R² is isoxazolyl substituted with one or more substituents independently selected from R⁷. In embodiments of a compound of Formula (VII), R² is thiazolyl substituted with one or more substituents independently selected from R⁷. In embodiments of a compound of Formula (VII), R² is isothiazolyl substituted with one or more substituents independently selected from R⁷. In embodiments of a compound of Formula (VII), R² is oxadiazolyl substituted with one or more substituents independently selected from R⁷. In embodiments of a compound of Formula (VII), R² is thiadiazolyl substituted with one or more substituents independently selected from R⁷.

In embodiments of a compound of Formula (VII), R¹ is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are optionally substituted with one or more substituents independently selected from R⁶ and R^{6′}. In embodiments of a compound of Formula (VII), R¹ is selected from bicyclic C₄₋₁₂cycloalkyl, bicyclic C₂₋₁₁heterocycloalkyl, bicyclic C₇₋₁₂ aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are optionally substituted with one or more substituents independently selected from R⁶. In embodiments of a compound of Formula (VII), R¹ is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are optionally substituted with one or more substituents independently selected from R^{6′}.

In embodiments of a compound of Formula (VII), R² is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are optionally substituted with one or more substituents independently selected from R⁷ and R^{7′}. In embodiments of a compound of Formula (VII), R² is selected from bicyclic C₄₋₁₂cycloalkyl, bicyclic C₂₋₁₁heterocycloalkyl, bicyclic C₇₋₁₂ aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are optionally substituted with one or more substituents independently selected from R^{7′}. In embodiments of a compound of Formula (VII), R² is selected from bicyclic C₄₋₁₂cycloalkyl, bicyclic C₂₋₁₁heterocycloalkyl, bicyclic C₇₋₁₂aryl, and bicyclic C₁₋₁₂heteroaryl, wherein bicyclic C₄₋₁₂cycloalkyl, bicyclic C₂₋₁₁heterocycloalkyl, bicyclic C₇₋₁₂aryl, and bicyclic C₁₋₁₂heteroaryl are optionally substituted with one or more substituents independently selected from R⁷.

In embodiments of a compound of Formula (VII), R¹ is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are optionally substituted with one or more substituents independently selected from R⁶ and R^{6′}. In embodiments of a compound of Formula (VII), R¹ is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are optionally substituted with one or more substituents independently selected from R⁶. In embodiments of a compound of Formula (VII), R¹ is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are optionally substituted with one or more substituents independently selected from R^{6′}.

In embodiments of a compound of Formula (VII), R² is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are optionally substituted with one or more substituents independently selected from R⁷ and R^{7′}. In embodiments of a compound of Formula (VII), R² is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are optionally substituted with one or more substituents independently selected from R⁷. In embodiments of a compound of Formula (VII), R² is selected from indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl, wherein indazolyl, 7-azaindazolyl, 6-azaindazolyl, 5-azaindazolyl, 4-azaindazolyl, benzoxazolyl, benzthiazolyl, naphthyl, indenyl, indolinyl, indolyl, isoindolyl, indolizinyl, benzimidazolyl, 4-azaindolyl, 5-azaindolyl, 6-azaindolyl, 7-azaindolyl, purinyl, benzofuranyl, isobenzofuranyl, benzo[c]thiophenyl, benzo[b]thiophenyl, benzoisoxazolyl, benzoisothiazolyl, quinolinyl, isoquinolinyl, quinoxalinyl, phthalazinyl, quinazolinyl, cinnolinyl, naphthyridinyl, pyrido[3,2-d]pyrimidinyl, pyrido[4,3-d]pyrimidinyl, pyrido[3,4-d]pyrazinyl, pyrido[2,3-d]pyrazinyl, and pteridinyl are optionally substituted with one or more substituents independently selected from R^{7′}.

In some embodiments, R¹ is C₅₋₁₂cycloalkyl substituted with one or more substituents independently selected from R⁶ and R^{6′}. In some embodiments, R¹ is 5 to 12 membered heterocycloalkyl substituted with one or more substituents independently selected from R⁶ and R^{6′}, wherein ring heteroatoms of the 5 to 12 membered heterocycloalkyl are independently selected from N, O, and S. In some embodiments, R¹ is C₆₋₁₂aryl substituted with one or more substituents independently selected from R⁶ and R^{6′}. In some embodiments, R¹ is 5 to 12 membered heteroaryl substituted with one or more substituents independently selected from R⁶ and R^{6′}, wherein ring heteroatoms of the 5 to 12 membered heteroaryl are independently selected from N, O, and S.

In some embodiments, R^{1a} is hydrogen.

In some embodiments, R² is C₅₋₁₂cycloalkyl substituted with one or more substituents independently selected from R⁷ and R^{7′}. In some embodiments, R² is 5 to 12 membered heterocycloalkyl substituted with one or more substituents independently selected from R⁷ and R^{7′}, wherein ring heteroatoms of the 5 to 12 membered heterocycloalkyl are independently selected from N, O, and S. In some embodiments, R² is C₆₋₁₂aryl substituted with one or more substituents independently selected from R⁷ and R^{7′}. In some embodiments, R² is 5 to 12 membered heteroaryl substituted with one or more substituents independently selected from R⁷ and R^{7′}, wherein ring heteroatoms of the 5 to 12 membered heteroaryl are independently selected from N, O, and S.

In some embodiments, R² is hydrogen.

In some embodiments, R³ is halogen and R⁴ is —OR¹². In some embodiments, R³ is halogen and R⁴ is —OH. In some embodiments, R³ is F and R⁴ is —OH. In some embodiments, R³ is Cl and R⁴ is —OH.

In some embodiments, R⁵ is hydrogen. In some embodiments, R⁵ is halogen. In some embodiments, R⁵ is C₁₋₆alkyl. In some embodiments, R⁵ is —OH.

In some embodiments, R⁶ is independently selected from halogen, oxo, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R²⁶.

In some embodiments, R⁶ is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In some embodiments, R⁷ is independently selected from halogen, oxo, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R²⁶.

In some embodiments, R⁷ is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In some embodiments, R^{6′} is independently selected from —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In some embodiments, R^{6′} is —CN. In some embodiments, R^{6′} is C₁₋₆alkyl. In some embodiments, R^{6′} is C₂₋₆alkenyl. In some embodiments, R^{6′} is C₂₋₆alkynyl. In some embodiments, R^{6′} is C₃₋₁₀cycloalkyl. In some embodiments, R^{6′} is C₂₋₉heterocycloalkyl. In some embodiments, R^{6′} is C₆₋₁₀aryl. In some embodiments, R^{6′} is C₁₋₉heteroaryl. In some embodiments, R^{6′} is —OR¹². In some embodiments, R^{6′} is —C(O)OR¹². In some embodiments, R^{6′} is —OC(O)N(R¹²)(R¹³). In some embodiments, R^{6′} is —C(O)R¹⁵. In some embodiments, R^{6′} is —OC(O)R¹⁵. In some embodiments, R^{6′} is —C(O)N(R¹²)(R¹³). In some embodiments, R^{6′} is —C(O)C(O)N(R¹²)(R¹³). In some embodiments, R^{6′} is —CH₂C(O)N(R¹²)(R¹³). In some embodiments, R^{6′} is —CH₂N(R¹⁴)C(O)R¹⁵. In some embodiments, R^{6′} is —CH₂S(O)₂R¹⁵. In some embodiments, R^{6′} is C₁₋₆alkyl optionally substituted with one, two, or three R²⁶. In some embodiments, R^{6′} is C₂₋₆alkenyl optionally substituted with one, two, or three R²⁶. In some embodiments, R^{6′} is C₂₋₆alkynyl optionally substituted with one, two, or three R²⁶. In some embodiments, R^{6′} is C₃₋₁₀cycloalkyl optionally substituted with one, two, or three R²⁶. In some embodiments, R^{6′} is C₂₋₉heterocycloalkyl optionally substituted with one, two, or three R²⁶. In some embodiments, R^{6′} is C₆₋₁₀aryl optionally substituted with one, two, or three R²⁶. In some embodiments, R^{6′} is C₁₋₉heteroaryl optionally substituted with one, two, or three R²⁶. In some embodiments, R^{6′} is C₁₋₆alkyl substituted with one, two, or three R²⁶. In some embodiments, R^{6′} is C₂₋₆alkenyl substituted with one, two, or three R²⁶. In some embodiments, R^{6′} is C₂₋₆alkynyl substituted with one, two, or three R²⁶. In some embodiments, R^{6′} is C₃₋₁₀cycloalkyl substituted with one, two, or three R²⁶. In some embodiments, R^{6′} is C₂₋₉heterocycloalkyl substituted with one, two, or three R²⁶. In some embodiments, R^{6′} is C₆₋₁₀aryl substituted with one, two, or three R²⁶. In some embodiments, R^{6′} is C₁₋₉heteroaryl substituted with one, two, or three R²⁶.

In some embodiments, R^{6′} is C₁₋₆alkyl-R²⁶. In some embodiments, R^{6′} is C₆alkyl-R²⁶. In some embodiments, R^{6′} is C₅alkyl-R²⁶. In some embodiments, R^{6′} is C₄alkyl-R²⁶. In some embodiments, R^{6′} is C₃alkyl-R²⁶. In some embodiments, R^{6′} is C₂alkyl-R²⁶. In some embodiments, R^{6′} is —CH₂—R²⁶.

In some embodiments, R^{6′} is C₁₋₆alkyl-C₃₋₆cycloalkyl, wherein C₃₋₆cycloalkyl is optionally substituted with one, two or three independently selected R²⁷. In some embodiments, R^{6′} is C₆alkyl-C₃₋₆cycloalkyl, wherein C₃₋₆cycloalkyl is optionally substituted with one, two or three independently selected R²⁷. In some embodiments, R^{6′} is C₅alkyl-C₃₋₆cycloalkyl, wherein C₃₋₆cycloalkyl is optionally substituted with one, two or three independently selected R²⁷. In some embodiments, R^{6′} is C₄alkyl-C₃₋₆cycloalkyl, wherein C₃₋₆cycloalkyl is optionally substituted with one, two or three independently selected R²⁷. In some embodiments, R^{6′} is C₃alkyl-C₃₋₆cycloalkyl, wherein C₃₋₆cycloalkyl is optionally substituted with one, two or three independently selected R²⁷. In some embodiments, R^{6′} is C₂alkyl-C₃₋₆cycloalkyl, wherein C₃₋₆cycloalkyl is optionally substituted with one, two or three independently selected R²⁷. In some embodiments, R^{6′} is —CH₂—C₃₋₆cycloalkyl, wherein C₃₋₆cycloalkyl is optionally substituted with one, two or three independently selected R²⁷.

In some embodiments, R^{6′} is C₁₋₆alkyl-C₂₋₉heterocycloalkyl, wherein C₂₋₉heterocycloalkyl is optionally substituted with one, two or three independently selected R²⁷. In some embodiments, R^{6′} is C₆alkyl-C₂₋₉heterocycloalkyl, wherein C₂₋₉heterocycloalkyl is optionally substituted with one, two or three independently selected R²⁷. In some embodiments, R^{6′} is C₅alkyl-C₂₋₉heterocycloalkyl, wherein C₂₋₉heterocycloalkyl is optionally substituted with one, two or three independently selected R²⁷. In some embodiments, R^{6′} is C₄alkyl-C₂₋₉heterocycloalkyl, wherein C₂₋₉heterocycloalkyl is optionally substituted with one, two or three independently selected R²⁷. In some embodiments, R^{6′} is C₃alkyl-C₂₋₉heterocycloalkyl, wherein C₂₋₉heterocycloalkyl is optionally substituted with one, two or three independently selected R²⁷. In some embodiments, R^{6′} is C₂alkyl-C₂₋₉heterocycloalkyl, wherein C₂₋₉heterocycloalkyl is optionally substituted with one, two or three independently selected R²⁷. In some embodiments, R^{6′} is —CH₂—C₂₋₉heterocycloalkyl, wherein C₂₋₉heterocycloalkyl is optionally substituted with one, two or three independently selected R²⁷.

In some embodiments, R^{6′} is C₁₋₆alkyl-C₆₋₁₀aryl, wherein C₆₋₁₀aryl is optionally substituted with one, two or three independently selected R²⁷. In some embodiments, R^{6′} is C₆alkyl-C₆₋₁₀aryl, wherein C₆₋₁₀aryl is optionally substituted with one, two or three independently selected R²⁷. In some embodiments, R^{6′} is C₅alkyl-C₆₋₁₀aryl, wherein C₆₋₁₀aryl is optionally substituted with one, two or three independently selected R²⁷. In some embodiments, R^{6′} is C₄alkyl-C₆₋₁₀aryl, wherein C₆₋₁₀aryl is optionally substituted with one, two or three independently selected R²⁷. In some embodiments, R^{6′} is C₃alkyl-C₆₋₁₀aryl, wherein C₆₋₁₀aryl is optionally substituted with one, two or three independently selected R²⁷. In some embodiments, R^{6′} is C₂alkyl-C₆₋₁₀aryl, wherein C₆₋₁₀aryl is optionally substituted with one, two or three independently selected R²⁷. In some embodiments, R^{6′} is —CH₂—C₆₋₁₀aryl, wherein C₆₋₁₀aryl is optionally substituted with one, two or three independently selected R²⁷.

In some embodiments, R^{6′} is C₁₋₆alkyl-C₁₋₉heteroaryl, wherein C₁₋₉heteroaryl is optionally substituted with one, two or three independently selected R²⁷. In some embodiments, R^{6′} is C₆alkyl-C₁₋₉heteroaryl, wherein C₁₋₉heteroaryl is optionally substituted with one, two or three independently selected R²⁷. In some embodiments, R^{6′} is C₅alkyl-C₁₋₉heteroaryl, wherein C₁₋₉heteroaryl is optionally substituted with one, two or three independently selected R²⁷. In some embodiments, R^{6′} is C₄alkyl-C₁₋₉heteroaryl, wherein C₁₋₉ heteroaryl is optionally substituted with one, two or three independently selected R²⁷. In some embodiments, R^{6′} is C₃alkyl-C₁₋₉ heteroaryl, wherein C₁₋₉heteroaryl is optionally substituted with one, two or three independently selected R²⁷. In some embodiments, R^{6′} is C₂alkyl-C₁₋₉heteroaryl, wherein C₁₋₉heteroaryl is optionally substituted with one, two or three independently selected R²⁷. In some embodiments, R^{6′} is —CH₂—C₁₋₉heteroaryl, wherein C₁₋₉heteroaryl is optionally substituted with one, two or three independently selected R²⁷.

In some embodiments, R^{7′} is independently selected from —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

In some embodiments, R^{7′} is —CN. In some embodiments, R^{7′} is C₁₋₆alkyl. In some embodiments, R^{7′} is C₂₋₆alkenyl. In some embodiments, R^{7′} is C₂₋₆alkynyl. In some embodiments, R^{7′} is C₃₋₁₀cycloalkyl. In some embodiments, R^{7′} is C₂₋₉heterocycloalkyl. In some embodiments, R^{7′} is C₆₋₁₀aryl. In some embodiments, R^{7′} is C₁₋₉heteroaryl. In some embodiments, R^{7′} is —OR¹². In some embodiments, R^{7′} is —C(O)OR¹². In some embodiments, R^{7′} is —OC(O)N(R¹²)(R¹³). In some embodiments, R^{7′} is —C(O)R¹⁵. In some embodiments, R^{7′} is —OC(O)R¹⁵. In some embodiments, R^{7′} is —C(O)N(R¹²)(R¹³). In some embodiments, R^{7′} is —C(O)C(O)N(R¹²)(R¹³). In some embodiments, R^{7′} is —CH₂C(O)N(R¹²)(R¹³). In some embodiments, R^{7′} is —CH₂N(R¹⁴)C(O)R¹⁵. In some embodiments, R^{7′} is —CH₂S(O)₂R¹⁵. In some embodiments, R^{7′} is C₁₋₆alkyl optionally substituted with one, two, or three R²⁶. In some embodiments, R^{7′} is C₂₋₆alkenyl optionally substituted with one, two, or three R²⁶. In some embodiments, R^{7′} is C₂₋₆alkynyl optionally substituted with one, two, or three R²⁶. In some embodiments, R^{7′} is C₃₋₁₀cycloalkyl optionally substituted with one, two, or three R²⁶. In some embodiments, R^{7′} is C₂₋₉heterocycloalkyl optionally substituted with one, two, or three R²⁶. In some embodiments, R^{7′} is C₆₋₁₀aryl optionally substituted with one, two, or three R²⁶. In some embodiments, R^{7′} is C₁₋₉heteroaryl optionally substituted with one, two, or three R²⁶. In some embodiments, R^{7′} is C₁₋₆alkyl substituted with one, two, or three R²⁶. In some embodiments, R^{7′} is C₂₋₆alkenyl substituted with one, two, or three R²⁶. In some embodiments, R^{7′} is C₂₋₆alkynyl substituted with one, two, or three R²⁶. In some embodiments, R^{7′} is C₃₋₁₀cycloalkyl substituted with one, two, or three R²⁶. In some embodiments, R^{7′} is C₂₋₉heterocycloalkyl substituted with one, two, or three R²⁶. In some embodiments, R^{7′} is C₆₋₁₀aryl substituted with one, two, or three R²⁶. In some embodiments, R^{7′} is C₁₋₉heteroaryl substituted with one, two, or three R²⁶.

In some embodiments, R^{7′} is C₁₋₆alkyl-R²⁶. In some embodiments, R^{7′} is C₆alkyl-R²⁶. In some embodiments, R^{7′} is C₅alkyl-R²⁶. In some embodiments, R^{7′} is C₄alkyl-R²⁶. In some embodiments, R^{7′} is C₃alkyl-R²⁶. In some embodiments, R^{7′} is C₂alkyl-R²⁶. In some embodiments, R^{7′} is —CH₂—R²⁶.

In some embodiments, R^{7′} is C₁₋₆alkyl-C₃₋₆cycloalkyl, wherein C₃₋₆cycloalkyl is optionally substituted with one, two or three independently selected R²⁷. In some embodiments, R^{7′} is C₆alkyl-C₃₋₆cycloalkyl, wherein C₃₋₆cycloalkyl is optionally substituted with one, two or three independently selected R²⁷. In some embodiments, R^{7′} is C₅alkyl-C₃₋₆cycloalkyl, wherein C₃₋₆cycloalkyl is optionally substituted with one, two or three independently selected R²⁷. In some embodiments, R^{7′} is C₄alkyl-C₃₋₆cycloalkyl, wherein C₃₋₆cycloalkyl is optionally substituted with one, two or three independently selected R²⁷. In some embodiments, R^{7′} is C₃alkyl-C₃₋₆cycloalkyl, wherein C₃₋₆cycloalkyl is optionally substituted with one, two or three independently selected R²⁷. In some embodiments, R^{7′} is C₂alkyl-C₃₋₆cycloalkyl, wherein C₃₋₆cycloalkyl is optionally substituted with one, two or three independently selected R²⁷. In some embodiments, R^{7′} is —CH₂—C₃₋₆cycloalkyl, wherein C₃₋₆cycloalkyl is optionally substituted with one, two or three independently selected R²⁷.

In some embodiments, R^{7′} is C₁₋₆alkyl-C₂₋₉heterocycloalkyl, wherein C₂₋₉heterocycloalkyl is optionally substituted with one, two or three independently selected R²⁷. In some embodiments, R^{7′} is C₆alkyl-C₂₋₉heterocycloalkyl, wherein C₂₋₉heterocycloalkyl is optionally substituted with one, two or three independently selected R²⁷. In some embodiments, R^{7′} is C₅alkyl-C₂₋₉heterocycloalkyl, wherein C₂₋₉heterocycloalkyl is optionally substituted with one, two or three independently selected R²⁷. In some embodiments, R^{7′} is C₄alkyl-C₂₋₉heterocycloalkyl, wherein C₂₋₉heterocycloalkyl is optionally substituted with one, two or three independently selected R²⁷. In some embodiments, R^{7′} is C₃alkyl-C₂₋₉heterocycloalkyl, wherein C₂₋₉heterocycloalkyl is optionally substituted with one, two or three independently selected R²⁷. In some embodiments, R^{7′} is C₂alkyl-C₂₋₉heterocycloalkyl, wherein C₂₋₉heterocycloalkyl is optionally substituted with one, two or three independently selected R²⁷. In some embodiments, R^{7′} is —CH₂—C₂₋₉heterocycloalkyl, wherein C₂₋₉heterocycloalkyl is optionally substituted with one, two or three independently selected R²⁷.

In some embodiments, R^{7′} is C₁₋₆alkyl-C₆₋₁₀aryl, wherein C₆₋₁₀aryl is optionally substituted with one, two or three independently selected R²⁷. In some embodiments, R^{7′} is C₆alkyl-C₆₋₁₀aryl, wherein C₆₋₁₀aryl is optionally substituted with one, two or three independently selected R²⁷. In some embodiments, R^{7′} is C₅alkyl-C₆₋₁₀aryl, wherein C₆₋₁₀aryl is optionally substituted with one, two or three independently selected R²⁷. In some embodiments, R^{7′} is C₄alkyl-C₆₋₁₀aryl, wherein C₆₋₁₀aryl is optionally substituted with one, two or three independently selected R²⁷. In some embodiments, R^{7′} is C₃alkyl-C₆₋₁₀aryl, wherein C₆₋₁₀aryl is optionally substituted with one, two or three independently selected R²⁷. In some embodiments, R^{7′} is C₂alkyl-C₆₋₁₀aryl, wherein C₆₋₁₀aryl is optionally substituted with one, two or three independently selected R²⁷. In some embodiments, R^{7′} is —CH₂—C₆₋₁₀aryl, wherein C₆₋₁₀aryl is optionally substituted with one, two or three independently selected R²⁷.

In some embodiments, R^{7′} is C₁₋₆alkyl-C₁₋₉heteroaryl, wherein C₁₋₉heteroaryl is optionally substituted with one, two or three independently selected R²⁷. In some embodiments, R^{7′} is C₆alkyl-C₁₋₉heteroaryl, wherein C₁₋₉heteroaryl is optionally substituted with one, two or three independently selected R²⁷. In some embodiments, R^{7′} is C₅alkyl-C₁₋₉heteroaryl, wherein C₁₋₉heteroaryl is optionally substituted with one, two or three independently selected R²⁷. In some embodiments, R^{7′} is C₄alkyl-C₁₋₉heteroaryl, wherein C₁₋₉ heteroaryl is optionally substituted with one, two or three independently selected R²⁷. In some embodiments, R^{7′} is C₃alkyl-C₁₋₉ heteroaryl, wherein C₁₋₉heteroaryl is optionally substituted with one, two or three independently selected R²⁷. In some embodiments, R^{7′} is C₂alkyl-C₁₋₉heteroaryl, wherein C₁₋₉heteroaryl is optionally substituted with one, two or three independently selected R²⁷. In some embodiments, R^{7′} is —CH₂—C₁₋₉heteroaryl, wherein C₁₋₉heteroaryl is optionally substituted with one, two or three independently selected R²⁷.

In some embodiments, J¹ is N. In some embodiments, J¹ is C. In some embodiments, J¹ is C(R⁸).

In some embodiments, J² is N. In some embodiments, J² is N(R⁹). In some embodiments, J² is C(R⁹). In some embodiments, J² is C(R⁹)(R^{9a}). In some embodiments, J² is C(O).

In some embodiments, J³ is N(R¹⁰). In some embodiments, J³ is C(R¹⁰)(R^{10a}).

In some embodiments, R⁸ is hydrogen. In some embodiments, R⁸ is halogen. In some embodiments, R⁸ is —CN. In some embodiments, R⁸ is C₁₋₆alkyl optionally substituted with one, two, or three R^{20h}. In some embodiments, R⁸ is C₃₋₁₀cycloalkyl optionally substituted with one, two, or three R^{20h}. In some embodiments, R⁸ is C₂₋₉heterocycloalkyl optionally substituted with one, two, or three R^{20h}. In some embodiments, R⁸ is C₆₋₁₀aryl optionally substituted with one, two, or three R^{20h}. In some embodiments, R⁸ is C₁₋₉ heteroaryl optionally substituted with one, two, or three R^{20h}.

In some embodiments, R⁹ is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20l}. In some embodiments, R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰. In some embodiments, R⁹ is hydrogen. In some embodiments, R⁹ is halogen. In some embodiments, R⁹ is —CN. In some embodiments, R⁹ is C₁₋₆alkyl optionally substituted with one, two, or three R²⁰. In some embodiments, R⁹ is C₃₋₁₀cycloalkyl optionally substituted with one, two, or three R²¹. In some embodiments, R⁹ is C₂₋₉ heterocycloalkyl optionally substituted with one, two, or three R²⁰ⁱ. In some embodiments, R⁹ is C₆₋₁₀aryl optionally substituted with one, two, or three R²⁰ⁱ. In some embodiments, R⁹ is C₁₋₉heteroaryl optionally substituted with one, two, or three R²⁰ⁱ.

In some embodiments, R^{9a} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R². In some embodiments, R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ. In some embodiments, R^{9a} is hydrogen. In some embodiments, R^{9a} is halogen. In some embodiments, R^{9a} is —CN. In some embodiments, R^{9a} is C₁₋₆alkyl optionally substituted with one, two, or three R²⁰. In some embodiments, R^{9a} is C₃₋₁₀cycloalkyl optionally substituted with one, two, or three R²⁰ⁱ. In some embodiments, R^{9a} is C₂₋₉ heterocycloalkyl optionally substituted with one, two, or three R²⁰ⁱ. In some embodiments, R^{9a} is C₆₋₁₀aryl optionally substituted with one, two, or three R²⁰ⁱ. In some embodiments, R^{9a} is C₁₋₉heteroaryl optionally substituted with one, two, or three R²⁰ⁱ.

In some embodiments, R¹⁰ is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20j}. In some embodiments, R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j}. In some embodiments, R¹⁰ is hydrogen. In some embodiments, R¹⁰ is halogen. In some embodiments, R¹⁰ is —CN. In some embodiments, R¹⁰ is C₁₋₆alkyl optionally substituted with one, two, or three R^{20j}. In some embodiments, R¹⁰ is C₃₋₁₀cycloalkyl optionally substituted with one, two, or three R^{20j}. In some embodiments, R¹⁰ is C₂₋₉ heterocycloalkyl optionally substituted with one, two, or three R^{20j}. In some embodiments, R¹⁰ is C₆₋₁₀aryl optionally substituted with one, two, or three R^{20j}. In some embodiments, R¹⁰ is C₁₋₉heteroaryl optionally substituted with one, two, or three R^{20j}.

In some embodiments, R^{10a} is independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20j}. In some embodiments, R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j}. In some embodiments, R^{10a} is hydrogen. In some embodiments, R^{10a} is halogen. In some embodiments, R^{10a} is —CN. In some embodiments, R^{10a} is C₁₋₆alkyl optionally substituted with one, two, or three R^{20j}. In some embodiments, R^{10a} is C₃₋₁₀cycloalkyl optionally substituted with one, two, or three R^{20j}. In some embodiments, R^{10a} is C₂₋₉ heterocycloalkyl optionally substituted with one, two, or three R^{20j}. In some embodiments, R^{10a} is C₆₋₁₀aryl optionally substituted with one, two, or three R^{20j}. In some embodiments, R^{10a} is C₁₋₉heteroaryl optionally substituted with one, two, or three R^{20j}.

In embodiments, R¹ is triazolyl substituted with one or more substituents independently selected from R⁶ and R^{6′}. In embodiments, R¹ is pyrazolyl substituted with one or more substituents independently selected from R⁶ and R^{6′}. In embodiments, R¹ is pyrrolyl substituted with one or more substituents independently selected from R⁶ and R^{6′}. In embodiments, R¹ is imidazolyl substituted with one or more substituents independently selected from R⁶ and R^{6′}. In embodiments, R¹ is phenyl substituted with one or more substituents independently selected from R⁶ and R^{6′}. In embodiments, R¹ is pyridyl substituted with one or more substituents independently selected from R⁶ and R^{6′}. In embodiments, R¹ is pyrimidinyl substituted with one or more substituents independently selected from R⁶ and R^{6′}. In embodiments, R¹ is pyridazinyl substituted with one or more substituents independently selected from R⁶ and R^{6′}. In embodiments, R¹ is pyrazinyl substituted with one or more substituents independently selected from R⁶ and R^{6′}. In embodiments, R¹ is triazinyl substituted with one or more substituents independently selected from R⁶ and R^{6′}. In embodiments, R¹ is furanyl substituted with one or more substituents independently selected from R⁶ and R^{6′}. In embodiments, R¹ is thienyl substituted with one or more substituents independently selected from R⁶ and R^{6′}. In embodiments, R¹ is oxazolyl substituted with one or more substituents independently selected from R⁶ and R^{6′}. In embodiments, R¹ is isoxazolyl substituted with one or more substituents independently selected from R⁶ and R^{6′}. In embodiments, R¹ is thiazolyl substituted with one or more substituents independently selected from R⁶ and R^{6′}. In embodiments, R¹ is isothiazolyl substituted with one or more substituents independently selected from R⁶ and R^{6′}. In embodiments, R¹ is oxadiazolyl substituted with one or more substituents independently selected from R⁶ and R^{6′}. In embodiments, R¹ is thiadiazolyl substituted with one or more substituents independently selected from R⁶ and R^{6′}.

In embodiments, R¹ is triazolyl substituted with one or more substituents independently selected from R^{6′}. In embodiments, R¹ is pyrazolyl substituted with one or more substituents independently selected from R^{6′}. In embodiments, R¹ is pyrrolyl substituted with one or more substituents independently selected from R^{6′}. In embodiments, R¹ is imidazolyl substituted with one or more substituents independently selected from R^{6′}. In embodiments, R¹ is phenyl substituted with one or more substituents independently selected from R^{6′}. In embodiments, R¹ is pyridyl substituted with one or more substituents independently selected from R^{6′}. In embodiments, R¹ is pyrimidinyl substituted with one or more substituents independently selected from R^{6′}. In embodiments, R¹ is pyridazinyl substituted with one or more substituents independently selected from R^{6′}. In embodiments, R¹ is pyrazinyl substituted with one or more substituents independently selected from R^{6′}. In embodiments, R¹ is triazinyl substituted with one or more substituents independently selected from R^{6′}. In embodiments, R¹ is furanyl substituted with one or more substituents independently selected from R^{6′}. In embodiments, R¹ is thienyl substituted with one or more substituents independently selected from R^{6′}. In embodiments, R¹ is oxazolyl substituted with one or more substituents independently selected from R^{6′}. In embodiments, R¹ is isoxazolyl substituted with one or more substituents independently selected from R^{6′}. In embodiments, R¹ is thiazolyl substituted with one or more substituents independently selected from R^{6′}. In embodiments, R¹ is isothiazolyl substituted with one or more substituents independently selected from R^{6′}. In embodiments, R¹ is oxadiazolyl substituted with one or more substituents independently selected from R^{6′}. In embodiments, R¹ is thiadiazolyl substituted with one or more substituents independently selected from R^{6′}.

In embodiments, R¹ is triazolyl substituted with one or more substituents independently selected from R⁶. In embodiments, R¹ is pyrazolyl substituted with one or more substituents independently selected from R⁶. In embodiments, R¹ is pyrrolyl substituted with one or more substituents independently selected from R⁶. In embodiments, R¹ is imidazolyl substituted with one or more substituents independently selected from R⁶. In embodiments, R¹ is phenyl substituted with one or more substituents independently selected from R⁶. In embodiments, R¹ is pyridyl substituted with one or more substituents independently selected from R⁶. In embodiments, R¹ is pyrimidinyl substituted with one or more substituents independently selected from R⁶. In embodiments, R¹ is pyridazinyl substituted with one or more substituents independently selected from R⁶. In embodiments, R¹ is pyrazinyl substituted with one or more substituents independently selected from R⁶. In embodiments, R¹ is triazinyl substituted with one or more substituents independently selected from R⁶. In embodiments, R¹ is furanyl substituted with one or more substituents independently selected from R⁶. In embodiments, R¹ is thienyl substituted with one or more substituents independently selected from R⁶. In embodiments, R¹ is oxazolyl substituted with one or more substituents independently selected from R⁶. In embodiments, R¹ is isoxazolyl substituted with one or more substituents independently selected from R⁶. In embodiments, R¹ is thiazolyl substituted with one or more substituents independently selected from R⁶. In embodiments, R¹ is isothiazolyl substituted with one or more substituents independently selected from R⁶. In embodiments, R¹ is oxadiazolyl substituted with one or more substituents independently selected from R⁶. In embodiments, R¹ is thiadiazolyl substituted with one or more substituents independently selected from R⁶.

In embodiments, R² is triazolyl substituted with one or more substituents independently selected from R⁷ and R^{7′}. In embodiments, R² is pyrazolyl substituted with one or more substituents independently selected from R⁷ and R^{7′}. In embodiments, R² is pyrrolyl substituted with one or more substituents independently selected from R⁷ and R^{7′}. In embodiments, R² is imidazolyl substituted with one or more substituents independently selected from R⁷ and R^{7′}. In embodiments, R² is phenyl substituted with one or more substituents independently selected from R⁷ and R^{7′}. In embodiments, R² is pyridyl substituted with one or more substituents independently selected from R⁷ and R^{7′}. In embodiments, R² is pyrimidinyl substituted with one or more substituents independently selected from R⁷ and R^{7′}. In embodiments, R² is pyridazinyl substituted with one or more substituents independently selected from R⁷ and R^{7′}. In embodiments, R² is pyrazinyl substituted with one or more substituents independently selected from R⁷ and R^{7′}. In embodiments, R² is triazinyl substituted with one or more substituents independently selected from R⁷ and R^{7′}. In embodiments, R² is furanyl substituted with one or more substituents independently selected from R⁷ and R^{7′}. In embodiments, R² is thienyl substituted with one or more substituents independently selected from R⁷ and R^{7′}. In embodiments, R² is oxazolyl substituted with one or more substituents independently selected from R⁷ and R^{7′}. In embodiments, R² is isoxazolyl substituted with one or more substituents independently selected from R⁷ and R^{7′}. In embodiments, R² is thiazolyl substituted with one or more substituents independently selected from R⁷ and R^{7′}. In embodiments, R² is isothiazolyl substituted with one or more substituents independently selected from R⁷ and R^{7′}. In embodiments, R² is oxadiazolyl substituted with one or more substituents independently selected from R⁷ and R^{7′}. In embodiments, R² is thiadiazolyl substituted with one or more substituents independently selected from R⁷ and R^{7′}.

In embodiments, R² is triazolyl substituted with one or more substituents independently selected from R^{7′}. In embodiments, R² is pyrazolyl substituted with one or more substituents independently selected from R^{7′}. In embodiments, R² is pyrrolyl substituted with one or more substituents independently selected from R^{7′}. In embodiments, R² is imidazolyl substituted with one or more substituents independently selected from R^{7′}. In embodiments, R² is phenyl substituted with one or more substituents independently selected from R^{7′}. In embodiments, R² is pyridyl substituted with one or more substituents independently selected from R^{7′}. In embodiments, R² is pyrimidinyl substituted with one or more substituents independently selected from R^{7′}. In embodiments, R² is pyridazinyl substituted with one or more substituents independently selected from R^{7′}. In embodiments, R² is pyrazinyl substituted with one or more substituents independently selected from R^{7′}. In embodiments, R² is triazinyl substituted with one or more substituents independently selected from R^{7′}. In embodiments, R² is furanyl substituted with one or more substituents independently selected from R^{7′}. In embodiments, R² is thienyl substituted with one or more substituents independently selected from R^{7′}. In embodiments, R² is oxazolyl substituted with one or more substituents independently selected from R^{7′}. In embodiments, R² is isoxazolyl substituted with one or more substituents independently selected from R^{7′}. In embodiments, R² is thiazolyl substituted with one or more substituents independently selected from R^{7′}. In embodiments, R² is isothiazolyl substituted with one or more substituents independently selected from R^{7′}. In embodiments, R² is oxadiazolyl substituted with one or more substituents independently selected from R^{7′}. In embodiments, R² is thiadiazolyl substituted with one or more substituents independently selected from R^{7′}.

In embodiments, R² is triazolyl substituted with one or more substituents independently selected from R⁷. In embodiments, R² is pyrazolyl substituted with one or more substituents independently selected from R⁷. In embodiments, R² is pyrrolyl substituted with one or more substituents independently selected from R⁷. In embodiments, R² is imidazolyl substituted with one or more substituents independently selected from R⁷. In embodiments, R² is phenyl substituted with one or more substituents independently selected from R⁷. In embodiments, R² is pyridyl substituted with one or more substituents independently selected from R⁷. In embodiments, R² is pyrimidinyl substituted with one or more substituents independently selected from R⁷. In embodiments, R² is pyridazinyl substituted with one or more substituents independently selected from R⁷. In embodiments, R² is pyrazinyl substituted with one or more substituents independently selected from R⁷. In embodiments, R² is triazinyl substituted with one or more substituents independently selected from R⁷. In embodiments, R² is furanyl substituted with one or more substituents independently selected from R⁷. In embodiments, R² is thienyl substituted with one or more substituents independently selected from R⁷. In embodiments, R² is oxazolyl substituted with one or more substituents independently selected from R⁷. In embodiments, R² is isoxazolyl substituted with one or more substituents independently selected from R⁷. In embodiments, R² is thiazolyl substituted with one or more substituents independently selected from R⁷. In embodiments, R² is isothiazolyl substituted with one or more substituents independently selected from R⁷. In embodiments, R² is oxadiazolyl substituted with one or more substituents independently selected from R⁷. In embodiments, R² is thiadiazolyl substituted with one or more substituents independently selected from R⁷.

In embodiments, R¹ is

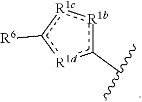

In embodiments, R¹ is

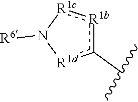

wherein R^{1c} is not N or N(R^{6c″}) when R^{1b} is C(R^{6b}) and R^{1d} is C(R^{6d}).

In embodiments, R¹ is

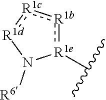

wherein R¹ is not a substituted pyrrolyl.

In embodiments, R¹ is

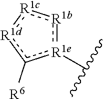

wherein R¹ is not a substituted imidazolyl wherein R^{1d} and R^{1e} are N.

In embodiments, R¹ is fluoro-substituted pyrazolyl optionally further substituted with one, two, or three substituents independently selected from R⁶ and R^{6′}. In embodiments, R^{1b} is O. In embodiments, R^{1b} is S. In embodiments, R^{1b} is N. In embodiments, R^{1b} is N(R^{6b″}). In embodiments, R^{1b} is C(R^{6b}). In embodiments, R^{1b} is C(R^{6b″})(R^{6b′}). In embodiments, R^{1c} is O. In embodiments, R^{1c} is S. In embodiments, R^{1c} is N. In embodiments, R^{1c} is N(R^{6c″}). In embodiments, R^{1c} is C(R^{6c}). In embodiments, R^{1c} is C(R^{6c})(R^{6c′}). In embodiments, R^{1d} is O. In embodiments, R^{1d} is S. In embodiments, R^{1d} is N. In embodiments, R^{1d} is N(R^{6d″}). In embodiments, R^{1d} is C(R^{6d}). In embodiments, R^{1d} is C(R^{6d})(R^{6d′}). In embodiments, R^{1e} is N. In embodiments, R^{1e} is C(R^{6e}).

In embodiments, R² is

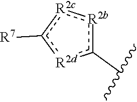

wherein R² is not a substituted imidazolyl wherein R^{2c} and R^{2d} are N.

In embodiments, R² is

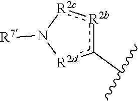

wherein R² is not a substituted pyrazolyl or pyrrolyl.

In embodiments, R² is

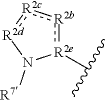

wherein R² is not a substituted pyrrolyl or imidazolyl.

In embodiments, R² is

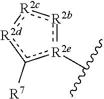

wherein R² is not a substituted imidazolyl wherein R^{2d} and R^{2e} are N, or R^{2b} and R^{2d}are N or NH.

In embodiments, R² is fluoro-substituted pyrazolyl optionally further substituted with one, two, or three substituents independently selected from R⁷ and R^{7′}.

In embodiments, R² is R^{2b} is O. In embodiments, R² is S. In embodiments, R² is N. In embodiments, R² is N(R^{7b″}). In embodiments, R² is C(R^{7h}). In embodiments, R² is C(R^{7b})(R^{7b′}). In embodiments, R^{2c} is O. In embodiments, R² is S. In embodiments, R² is N. In embodiments, R² is N(R^{7c″}). In embodiments, R² is, C(R^{7c}). In embodiments, R^{2c} is C(R^{7c})(R^{7c′}). In embodiments, R^{2d} is O. In embodiments, R^{2d} is S. In embodiments, R^{2d} is N. In embodiments, R^{2d} is N(R^{7d″}). In embodiments, R^{2d} is C(R^{7d}). In embodiments, R^{2d} is C(R^{7d})(R^{7d′}). In embodiments, R^{2e} is N. In embodiments, R^{2e} is C(R^{7e}).

In some embodiments, R¹ is bicyclic C₄₋₁₂cycloalkyl substituted with one or more substituents independently selected from R⁶ and R^{6′}. In some embodiments, R¹ is bicyclic C₂₋₁₁heterocycloalkyl substituted with one or more substituents independently selected from R⁶ and R^{6′}. In some embodiments, R¹ is bicyclic C₇₋₁₂aryl substituted with one or more substituents independently selected from R⁶ and R^{6′}. In some embodiments, R¹ is bicyclic C₁₋₁₂heteroaryl substituted with one or more substituents independently selected from R⁶ and R^{6′}.

In some embodiments, R¹ is bicyclic C₄₋₁₂cycloalkyl substituted with one or more R^{6′}. In some embodiments, R¹ is bicyclic C₂₋₁₁heterocycloalkyl substituted with one or more R^{6′}. In some embodiments, R¹ is bicyclic C₇₋₁₂aryl substituted with one or more R^{6′}. In some embodiments, R¹ is bicyclic C₁₋₁₂heteroaryl substituted with one or more R^{6′}.

In some embodiments, R¹ is bicyclic C₄₋₁₂cycloalkyl substituted with one or more R⁶. In some embodiments, R¹ is bicyclic C₂₋₁₁heterocycloalkyl substituted with one or more R⁶. In some embodiments, R¹ is bicyclic C₇₋₁₂aryl substituted with one or more R⁶. In some embodiments, R¹ is bicyclic C₁₋₁₂heteroaryl substituted with one or more R⁶.

In some embodiments, R¹ is bicyclic C₄₋₁₂cycloalkyl. In some embodiments, R¹ is bicyclic C₂₋₁₁heterocycloalkyl. In some embodiments, R¹ is bicyclic C₇₋₁₂aryl. In some embodiments, R¹ is bicyclic C₁₋₁₂heteroaryl.

In some embodiments, R² is bicyclic C₄₋₁₂cycloalkyl substituted with one or more substituents independently selected from R⁷ and R^{7′}. In some embodiments, R² is bicyclic C₂₋₁₁heterocycloalkyl substituted with one or more substituents independently selected from R⁷ and R^{7′}. In some embodiments, R² is bicyclic C₇₋₁₂aryl substituted with one or more substituents independently selected from R⁷ and R^{7′}. In some embodiments, R² is bicyclic C₁₋₁₂heteroaryl, substituted with one or more substituents independently selected from R⁷ and R^{7′}.

In some embodiments, R² is bicyclic C₄₋₁₂cycloalkyl substituted with one or more R^{7′}. In some embodiments, R² is bicyclic C₂₋₁₁heterocycloalkyl substituted with one or more R^{7′}. In some embodiments, R² is bicyclic C₇₋₁₂aryl substituted with one or more R^{7′}. In some embodiments, R² is bicyclic C₁₋₁₂heteroaryl, substituted with one or more R^{7′}.

In some embodiments, R² is bicyclic C₄₋₁₂cycloalkyl substituted with one or more R⁷. In some embodiments, R² is bicyclic C₂₋₁₁heterocycloalkyl substituted with one or more R⁷. In some embodiments, R² is bicyclic C₇₋₁₂aryl substituted with one or more R⁷. In some embodiments, R² is bicyclic C₁₋₁₂heteroaryl, substituted with one or more R⁷.

In some embodiments, R² is bicyclic C₄₋₁₂cycloalkyl. In some embodiments, R² is bicyclic C₂₋₁₁heterocycloalkyl. In some embodiments, R² is bicyclic C₇₋₁₂aryl. In some embodiments, R² is bicyclic C₁₋₁₂heteroaryl.

In some embodiments, R¹ is pyrazolyl substituted with one or more substituents independently selected from R⁶ and R^{6′}. In some embodiments, R² is pyrazolyl substituted with one or more substituents independently selected from R⁷ and R^{7′}. In some embodiments, R¹ is pyrazolyl substituted with one or more substituents independently selected from R^{6′}. In some embodiments, R² is pyrazolyl substituted with one or more substituents independently selected from R^{7′}.

In some embodiments, R¹ is pyrazolyl substituted with one R^{6′}. In some embodiments, R² is pyrazolyl substituted with one R^{7′}. In some embodiments, R¹ is

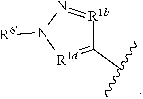

In some embodiments, R^{1b} is O. In some embodiments, R^{1b} is S. In some embodiments, R^{1b} is N. In some embodiments, R^{1b} is N(R^{6b″}). In some embodiments, R^{1b} is C(R⁶″). In some embodiments, R^{1b} is C(R^{6b})(R^{6b′}). In some embodiments, R^{1d} is O. In some embodiments, R^{1d} is S. In some embodiments, R^{1d} is N. In some embodiments, R^{1d} is N(R^{6d}″). In some embodiments, R^{1d} is C(R^{6d}). In some embodiments, R^{1d} is C(R^{6d})(R^{6d′}). In some embodiments, R^{6b}, R^{6b′}, R^{6d}, and R^{6d′} are independently hydrogen. In some embodiments, R^{6b}″ and R^{6d″} are independently hydrogen.

In some embodiments, R² is

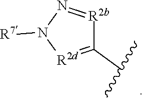

In some embodiments, R^{2b} is O. In some embodiments, R^{2b} is S. In some embodiments, R^{2b} is N. In some embodiments, R^{2b} is N(R^{7b}″). In some embodiments, R^{2b} is C(R^{7b}). In some embodiments, R^{2b} is C(R^{7b})(R^{7b′}). In some embodiments, R^{2d} is O. In some embodiments, R^{2d} is S. In some embodiments, R^{2d} is N. In some embodiments, R^{2d} is N(R^{7d″}). In some embodiments, R^{2d} is C(R^{7d}). In some embodiments, R^{2d} is C(R^{7d})(R^{7d′}).

In some embodiments, R¹⁶ is independently —C₁₋₆alkylene-OP(O)(OR^{16a})(OR^{6b}) wherein the C₁₋₆alkylene is optionally substituted with one, two, or three R²⁰″. In some embodiments, R¹⁶ is independently —P(O)(OR¹⁶)(OR^{6b}). In some embodiments, R¹⁶ is independently —C₁₋₆alkylene-OC(O)—R^{16c} wherein C₁₋₆alkylene is optionally substituted with one, two, or three R²⁰ⁿ. In some embodiments, R¹⁶ is independently —CH₂—OP(O)(OR¹⁶)(OR¹⁶). In some embodiments, R¹⁶ is independently —CH₂—OP(O)(OH)(OH). In some embodiments, R¹⁶ is independently —CH₂—OC(O)—R^{16c}.

In some embodiments, R¹ is selected from

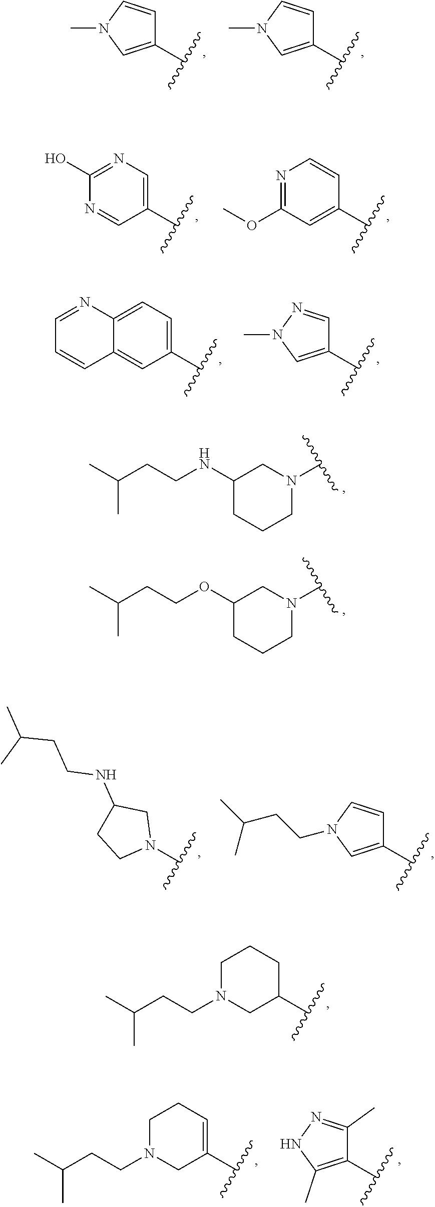

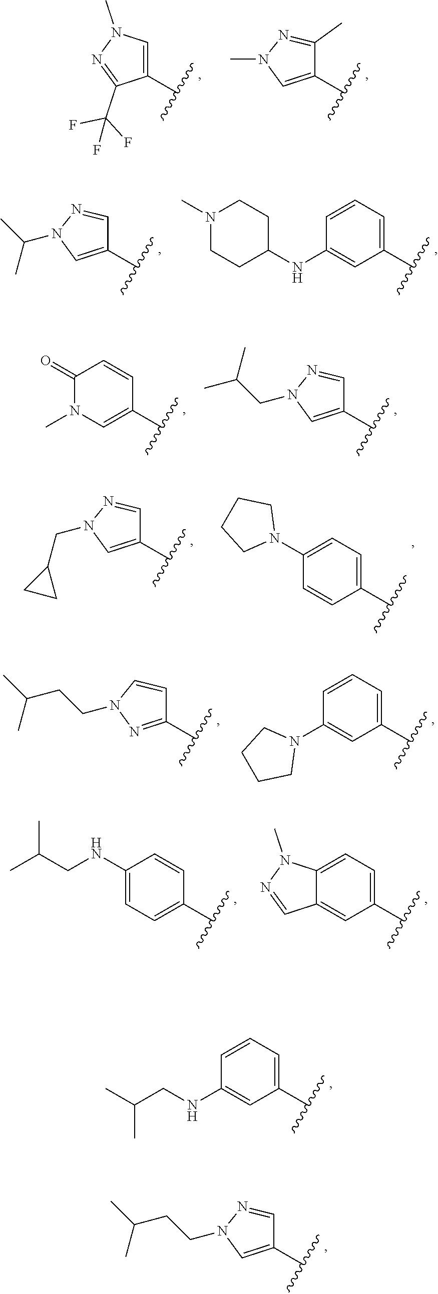

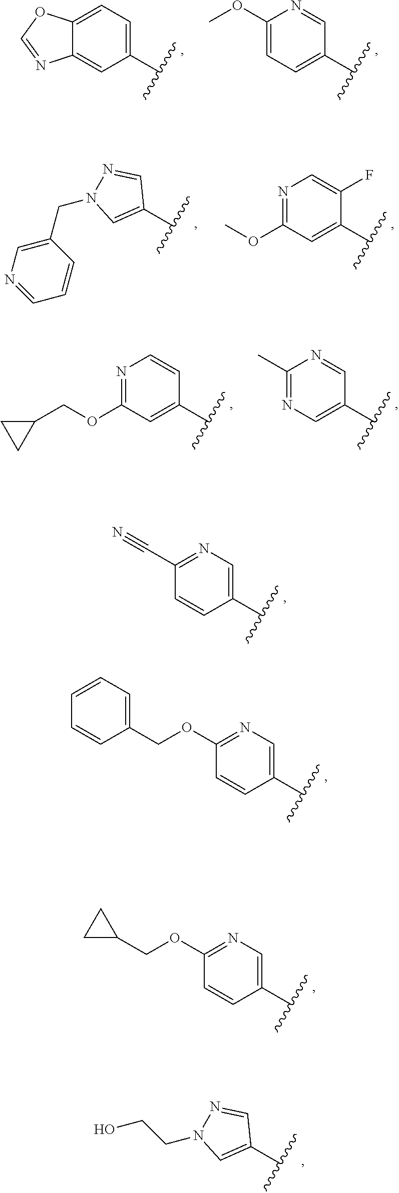

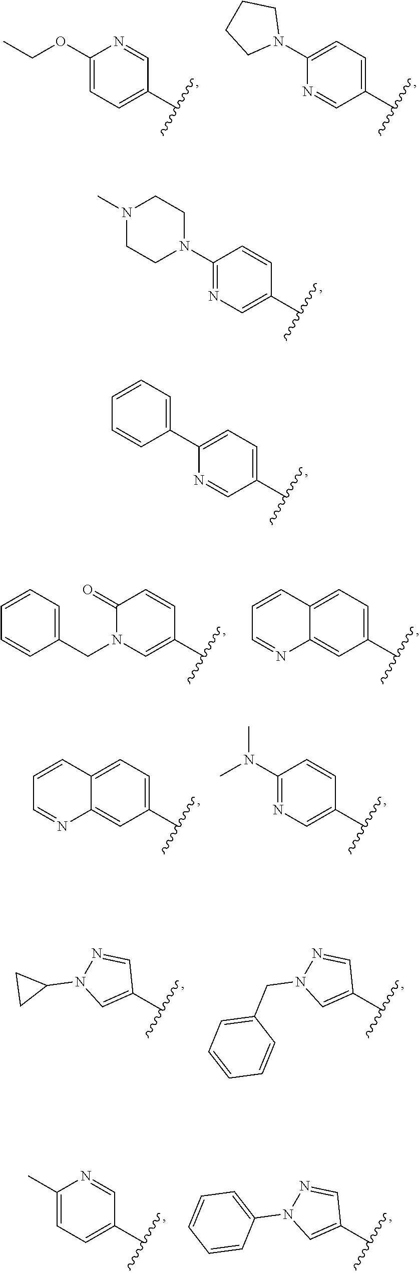

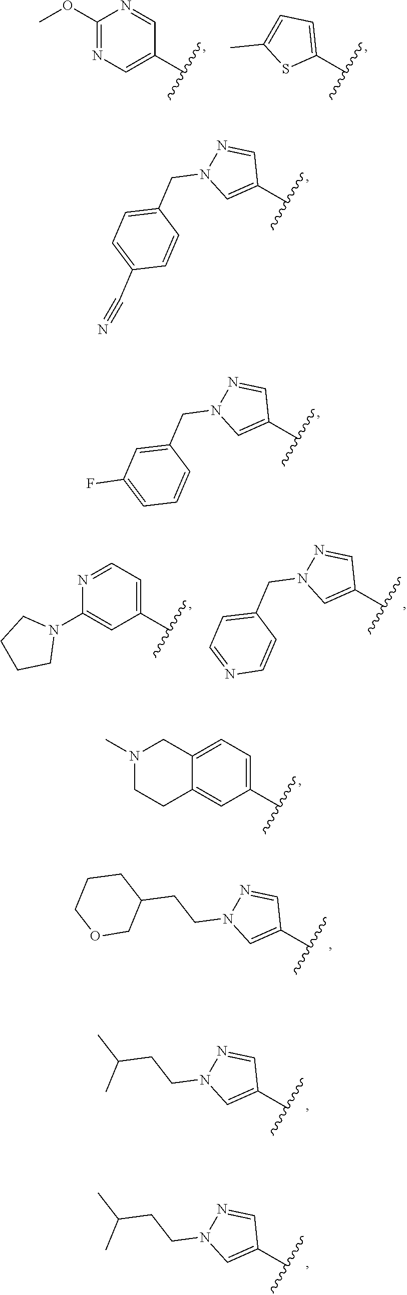

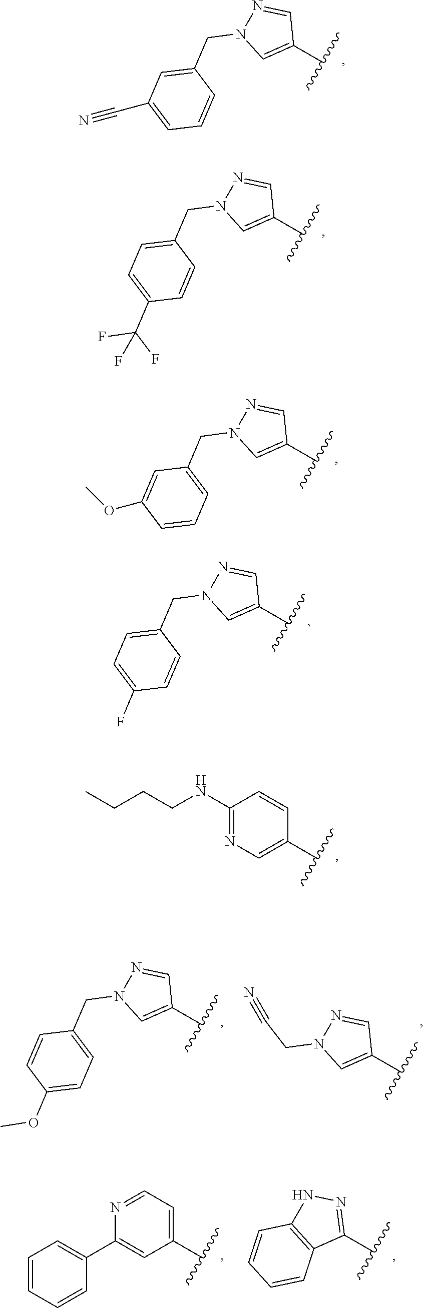

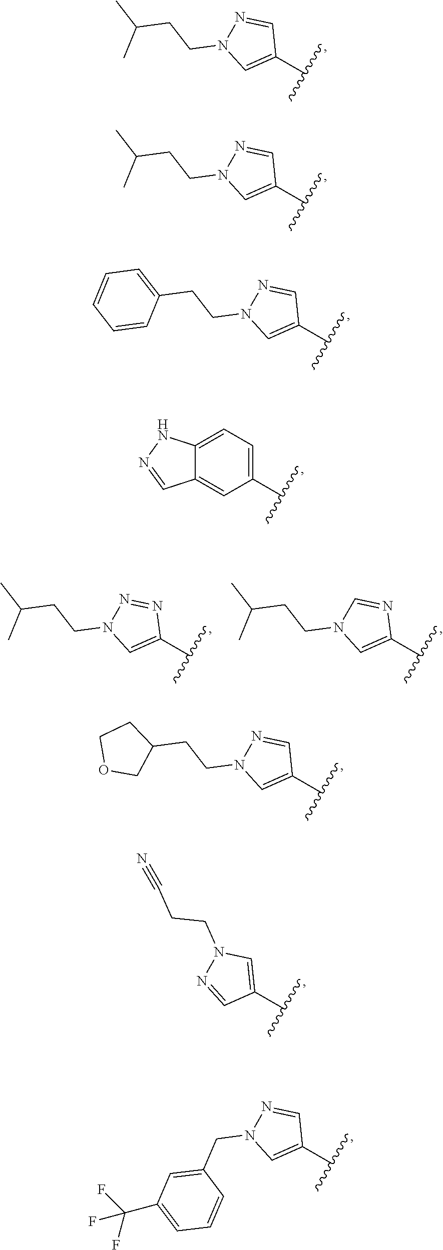

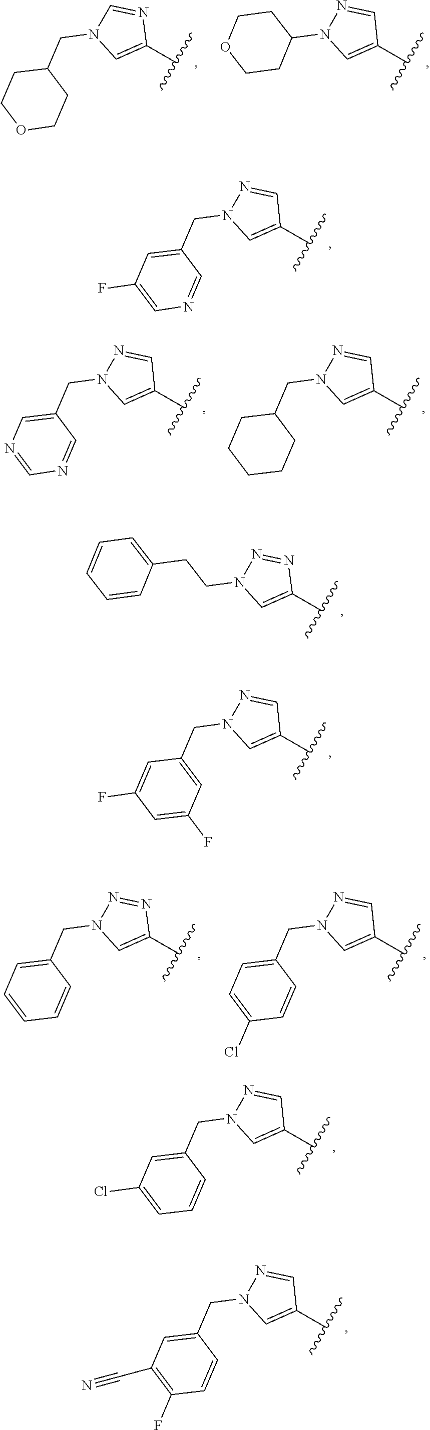

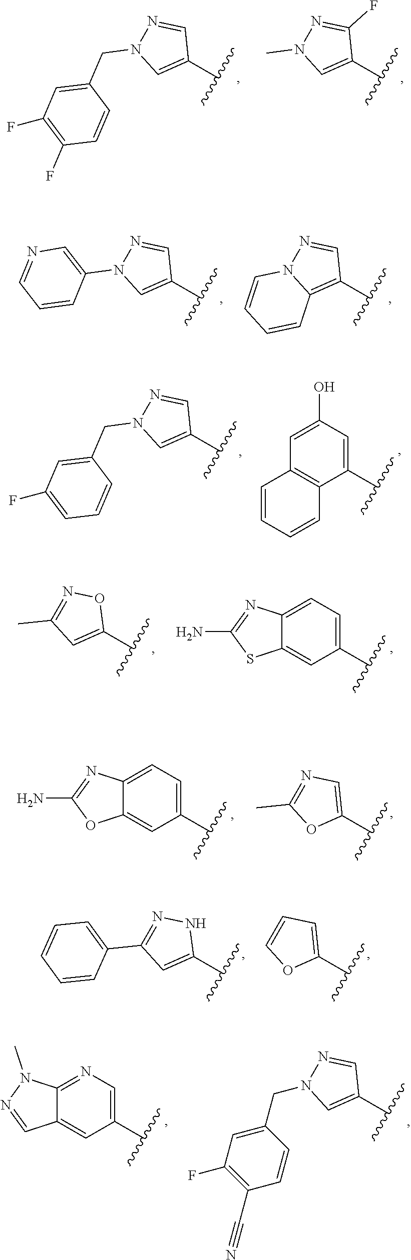

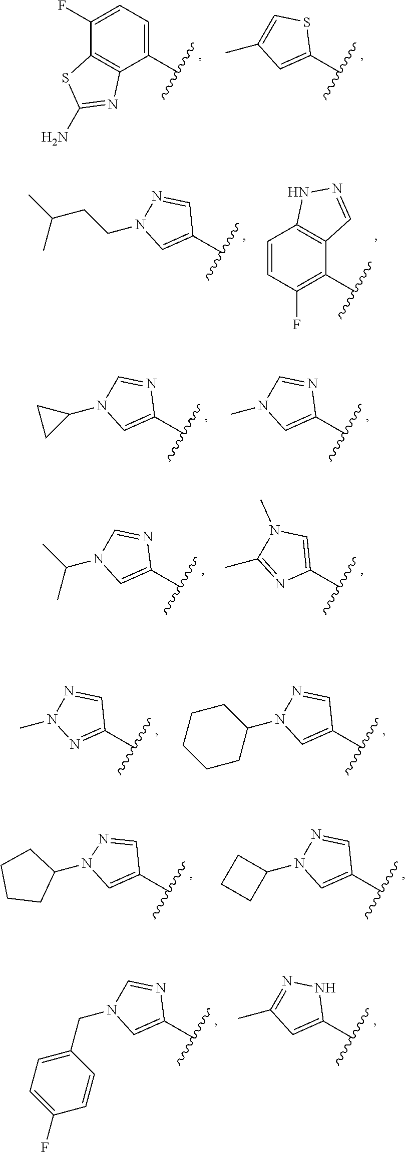

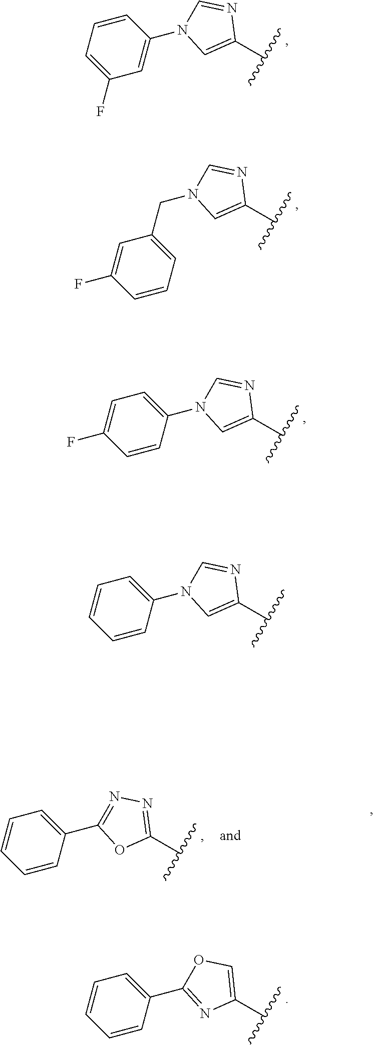 [SMILES: *c1cn(-c2cccc(F)c2)cn1 ; *c1cn(Cc2cccc(F)c2)cn1 ; *c1cn(-c2ccc(F)cc2)cn1 ; *c1cn(-c2ccccc2)cn1 ; *c1nnc(-c2ccccc2)o1 ; *c1coc(-c2ccccc2)n1]

In some embodiments, R² is selected from

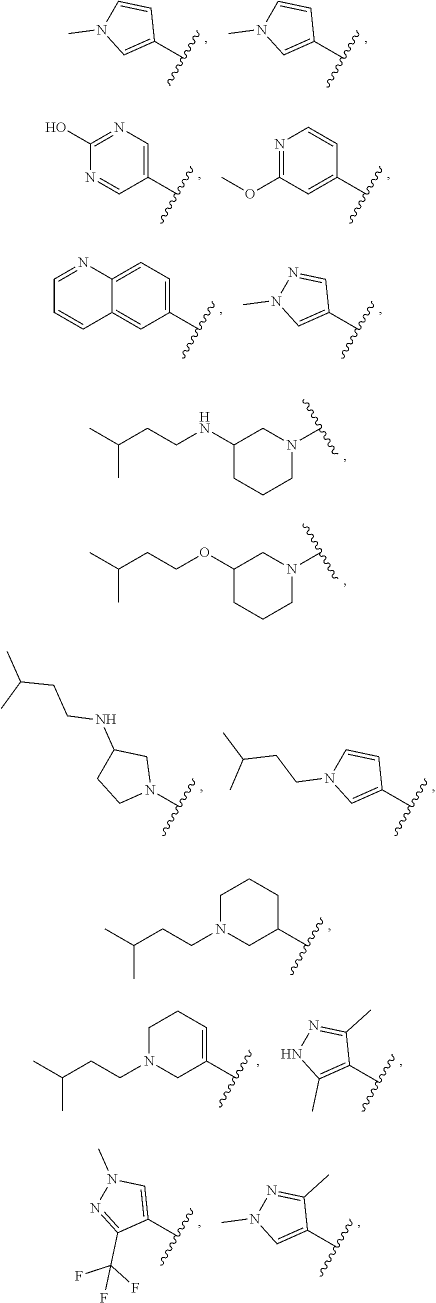

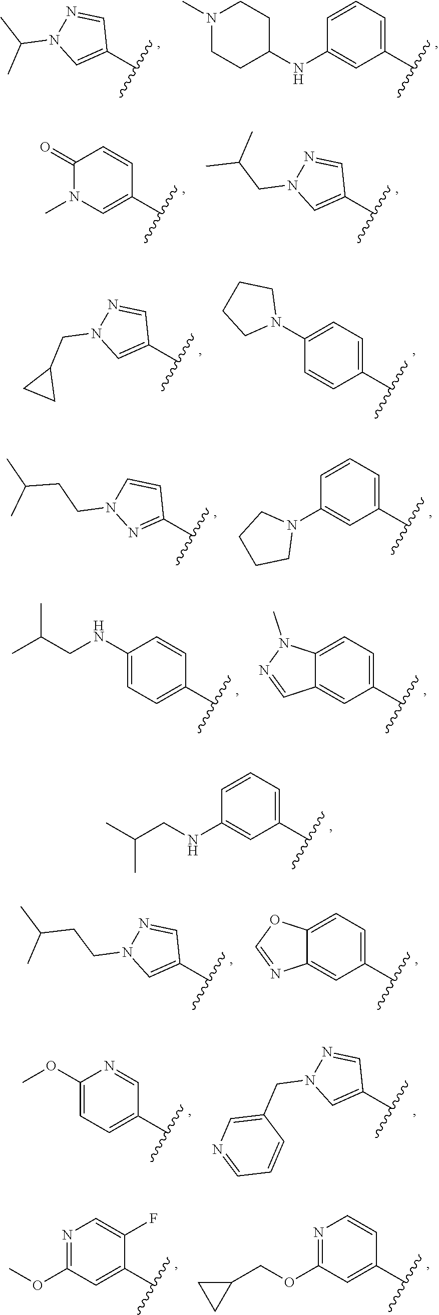

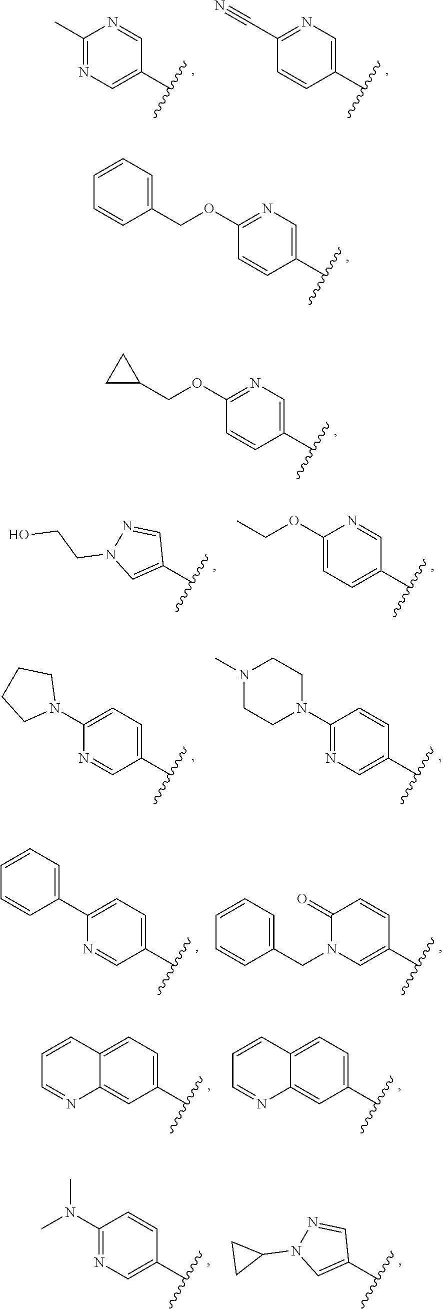

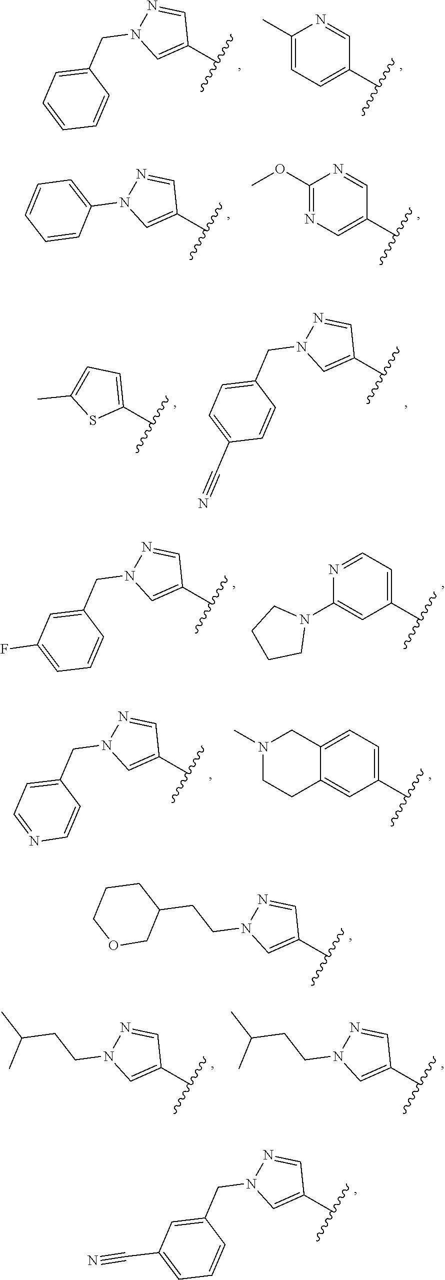

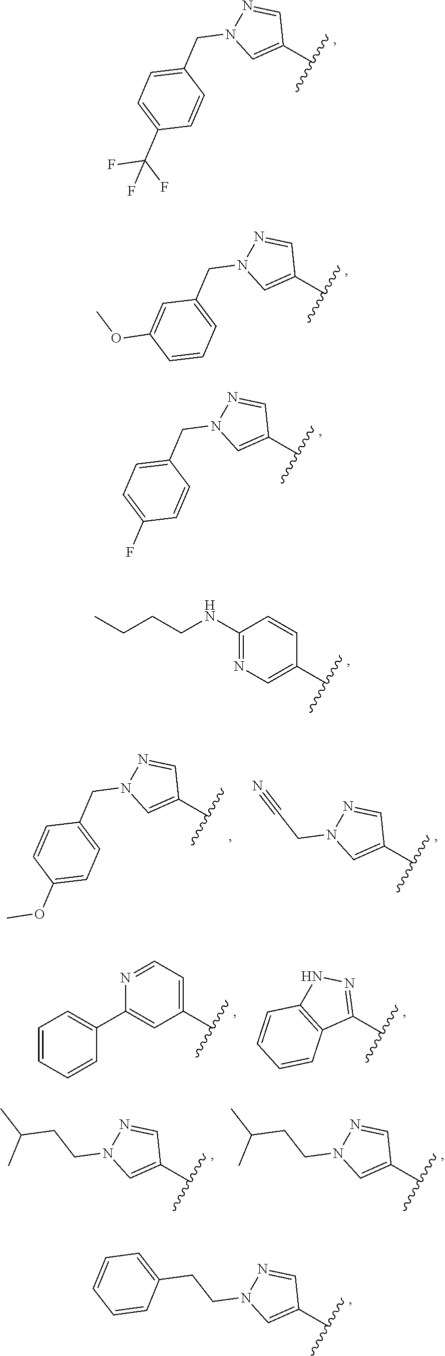

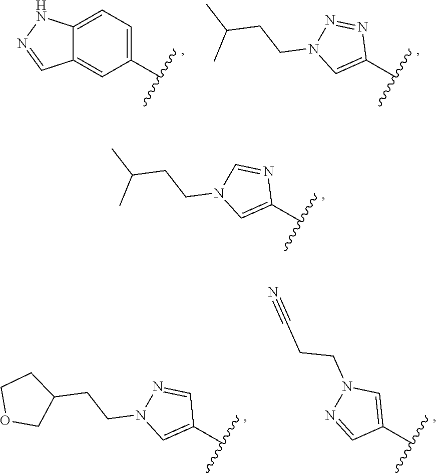 [SMILES: *c1ccc2[nH]ncc2c1 ; *c1cn(CCC(C)C)nn1 ; *c1cn(CCC(C)C)cn1 ; *c1cnn(CCC2CCOC2)c1 ; *c1cnn(CCC#N)c1]

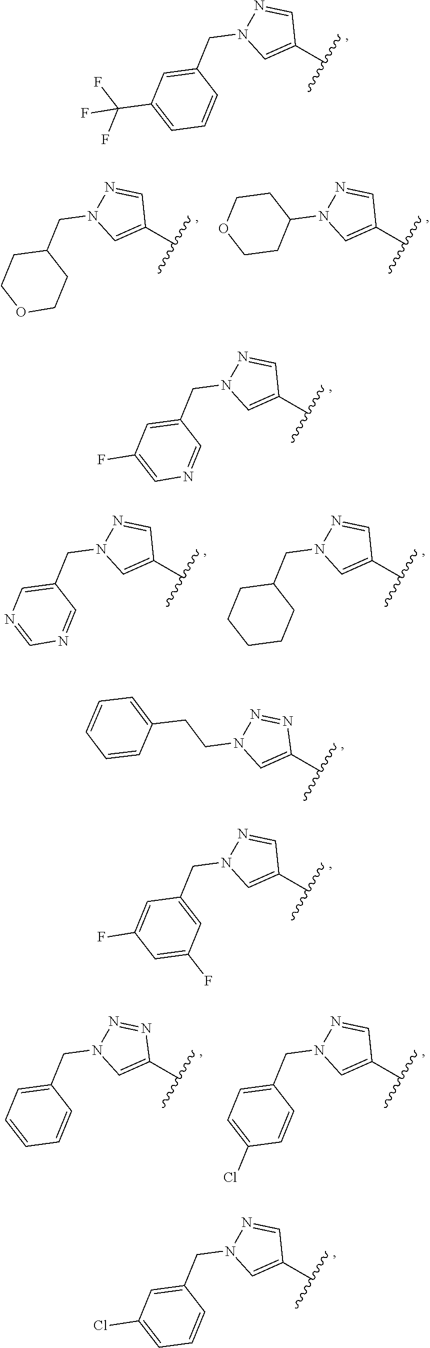

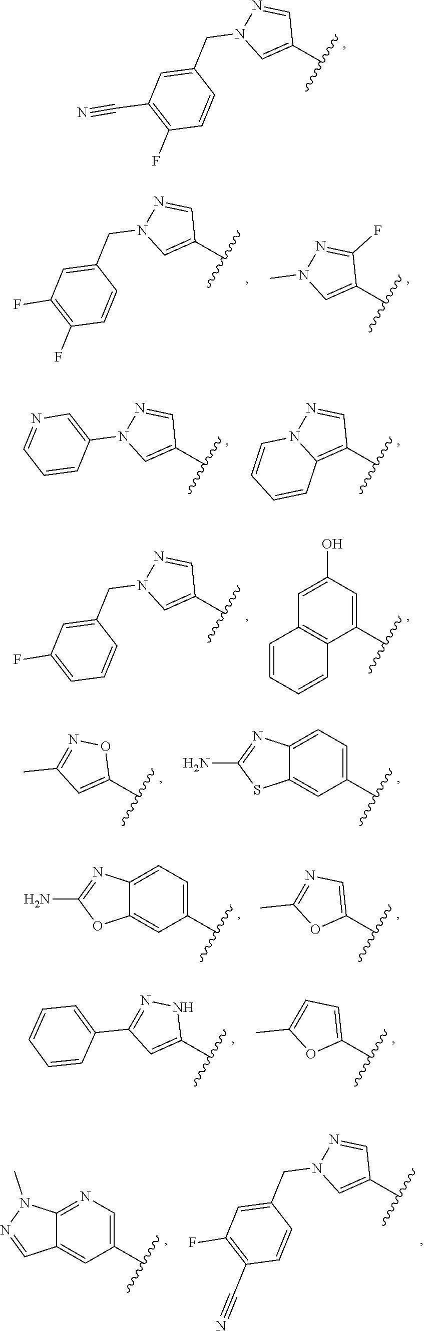

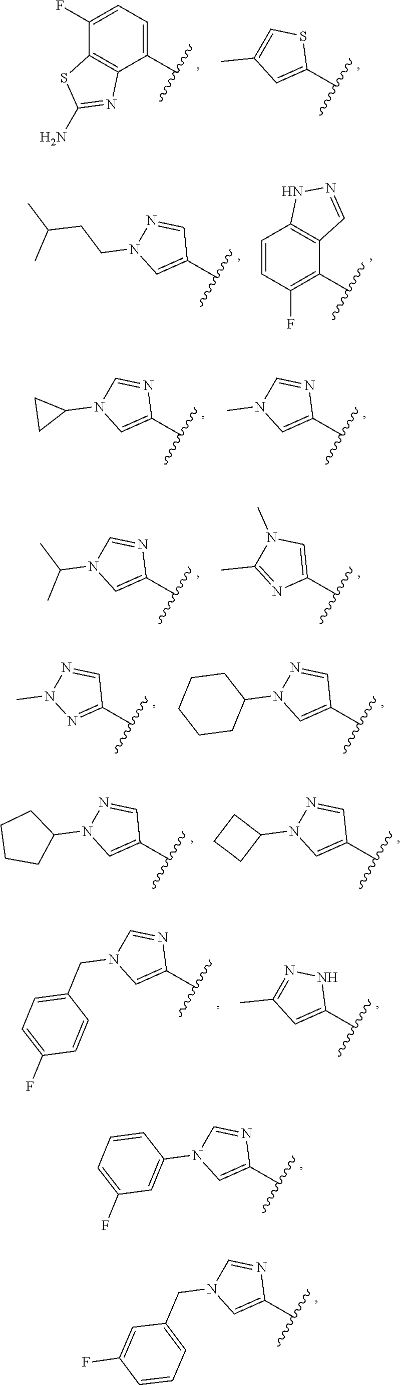

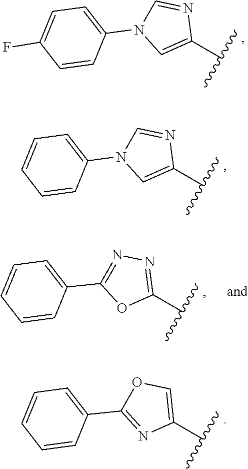 [SMILES: *c1cn(-c2ccc(F)cc2)cn1 ; *c1cn(-c2ccccc2)cn1 ; *c1nnc(-c2ccccc2)o1 ; *c1coc(-c2ccccc2)n1]

In embodiments, R⁶ is selected from methyl, —OH, methoxy, —NH(R¹²), —OR¹², pentyl, —CF₃, isopropyl, oxo, butyl, —CH₂— cyclopropyl, pyrrolidinyl, —CN, piperazinyl, methyl-piperazinyl, phenyl, benzyl, —N(CH₃)₂, fluoro-substituted phenyl, CN-substituted phenyl, CF₃-substituted phenyl, methoxy-substituted phenyl, —CH₂—CN, -ethyl-phenyl, ethyl-CN, pyrimidinyl, fluoro-substituted pyrimidinyl, cyclohexyl, halo-substituted phenyl, fluoro, chloro, pyridyl, —NH₂, cyclopentyl, and cyclobutyl.

In embodiments, R^{6′} is selected from methyl, —OH, methoxy, —NH(R¹²), —OR¹², pentyl, —CF₃, isopropyl, oxo, butyl, —CH₂— cyclopropyl, pyrrolidinyl, —CN, piperazinyl, methyl-piperazinyl, phenyl, benzyl, —N(CH₃)₂, fluoro-substituted phenyl, CN-substituted phenyl, CF₃-substituted phenyl, methoxy-substituted phenyl, —CH₂—CN, -ethyl-phenyl, ethyl-CN, pyrimidinyl, fluoro-substituted pyrimidinyl, cyclohexyl, halo-substituted phenyl, fluoro, chloro, pyridyl, —NH₂, cyclopentyl, and cyclobutyl.

In embodiments, R⁷ is selected from methyl, —OH, methoxy, —NH(R¹²), —OR¹², pentyl, —CF₃, isopropyl, oxo, butyl, —CH₂— cyclopropyl, pyrrolidinyl, —CN, piperazinyl, methyl-piperazinyl, phenyl, benzyl, —N(CH₃)₂, fluoro-substituted phenyl, CN-substituted phenyl, CF₃-substituted phenyl, methoxy-substituted phenyl, —CH₂—CN, -ethyl-phenyl, ethyl-CN, pyrimidinyl, fluoro-substituted pyrimidinyl, cyclohexyl, halo-substituted phenyl, fluoro, chloro, pyridyl, —NH₂, cyclopentyl, and cyclobutyl.

In embodiments, R^{7′} is selected from methyl, —OH, methoxy, —NH(R¹²), —OR¹², pentyl, —CF₃, isopropyl, oxo, butyl, —CH₂— cyclopropyl, pyrrolidinyl, —CN, piperazinyl, methyl-piperazinyl, phenyl, benzyl, —N(CH₃)₂, fluoro-substituted phenyl, CN-substituted phenyl, CF₃-substituted phenyl, methoxy-substituted phenyl, —CH₂—CN, -ethyl-phenyl, ethyl-CN, pyrimidinyl, fluoro-substituted pyrimidinyl, cyclohexyl, halo-substituted phenyl, fluoro, chloro, pyridyl, —NH₂, cyclopentyl, and cyclobutyl.

In embodiments, R^{6b}, R^{6b′}, R^{6c}, R^{6c′}, R^{6d}, R^{6d′}, R^{6e}, R^{6b″}, R^{6e″}, R^{6d″}, R^{7b}, R^{7b′}, R^{7c}, R^{7c′}, R^{7d}, R^{7d′}, R^{7e}, R^{7b″}, R^{7c″}, and R^{7d″} are independently selected from methyl, —OH, methoxy, —NH(R¹²), —OR¹², pentyl, —CF₃, isopropyl, oxo, butyl, —CH₂-cyclopropyl, pyrrolidinyl, —CN, piperazinyl, methyl-piperazinyl, phenyl, benzyl, —N(CH₃)₂, fluoro-substituted phenyl, CN-substituted phenyl, CF₃-substituted phenyl, methoxy-substituted phenyl, —CH₂—CN, -ethyl-phenyl, ethyl-CN, pyrimidinyl, fluoro-substituted pyrimidinyl, cyclohexyl, halo-substituted phenyl, fluoro, chloro, pyridyl, —NH₂, cyclopentyl, and cyclobutyl.

In embodiments, R^{6b}, R^{6b′}, R^{6c}, R^{6c′}, R^{6d}, R^{6d′}, R^{6e}, R^{6b″}, R^{6c″}, R^{6d″}, R^{7b}, R^{7b′}, R^{7c}, R^{7c′}, R^{7d}, R^{7d′}, R^{7e}, R^{7b″}, R^{7c″}, and R^{7d″} are independently selected from

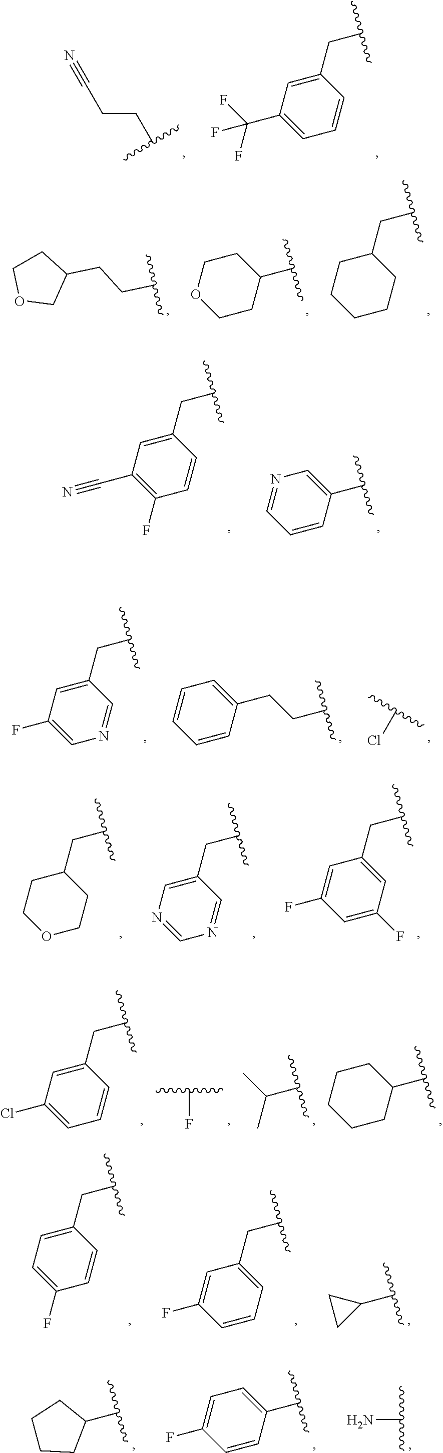

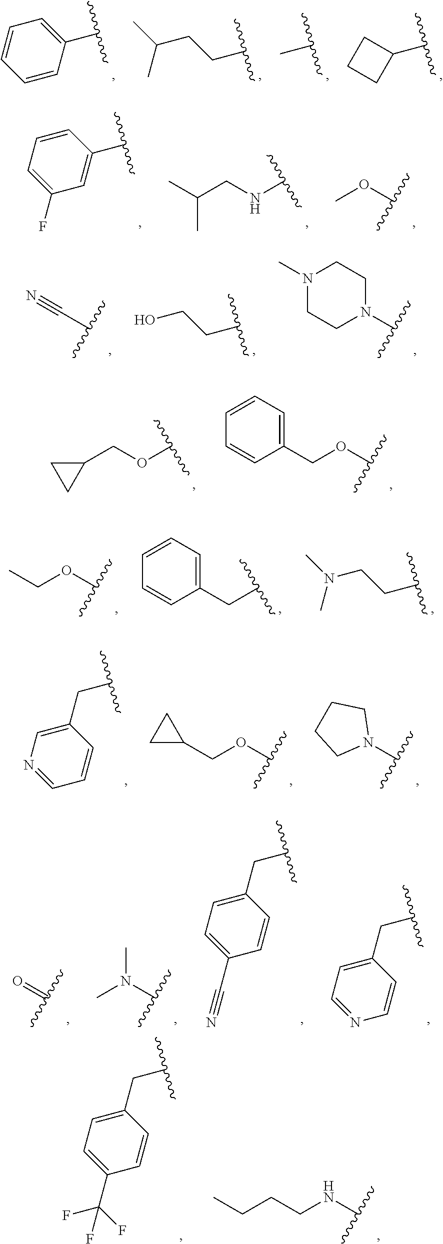

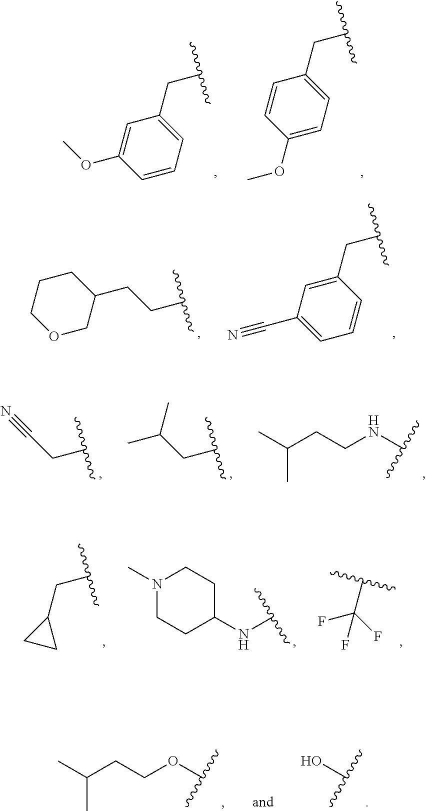

In embodiments, R⁶ is independently selected from

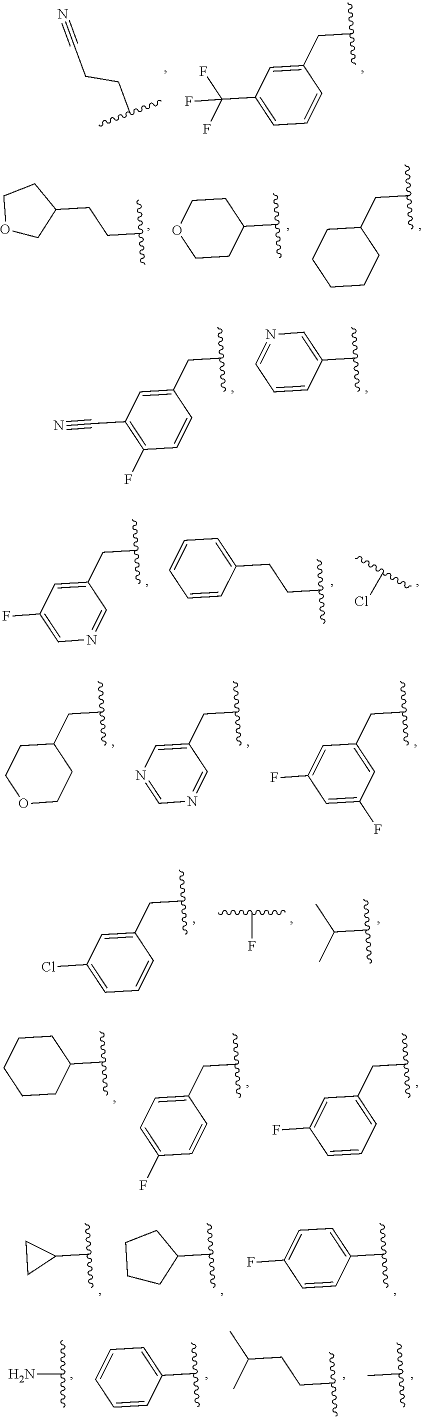

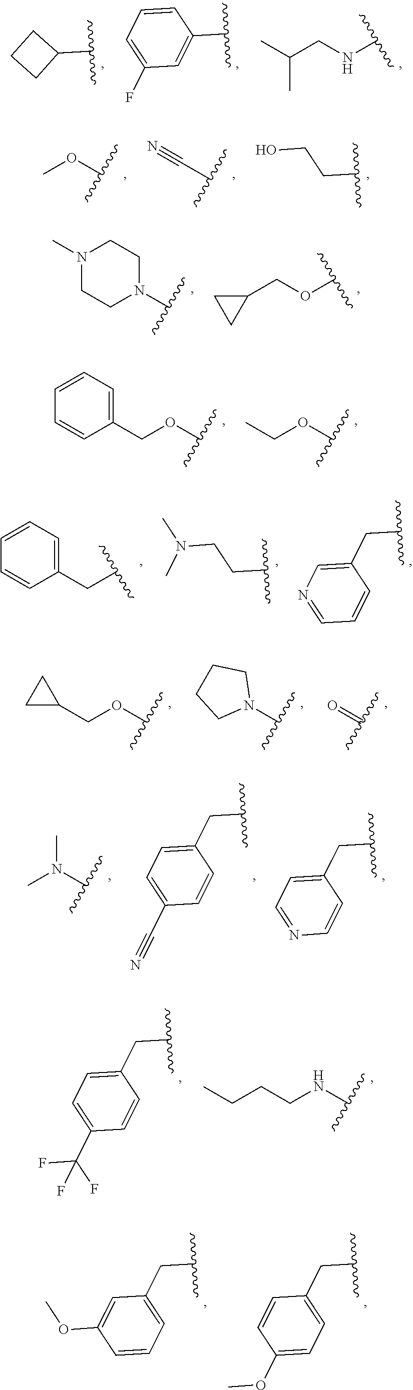

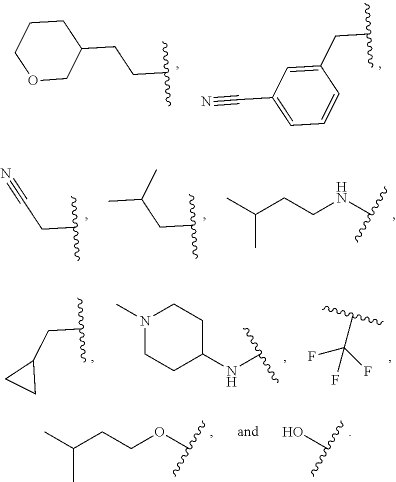

In embodiments, R^{6′} is independently selected from

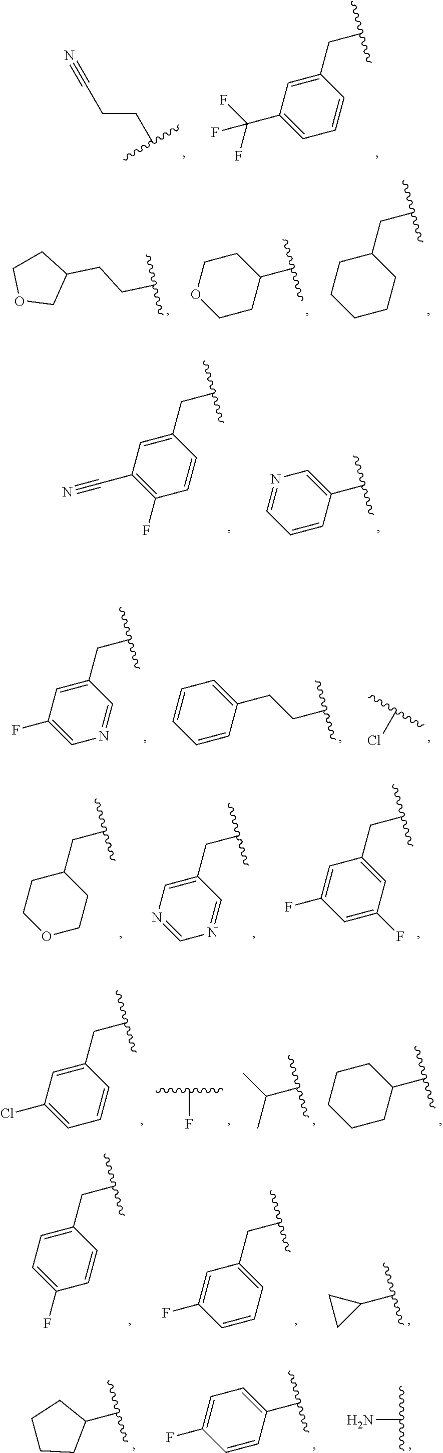

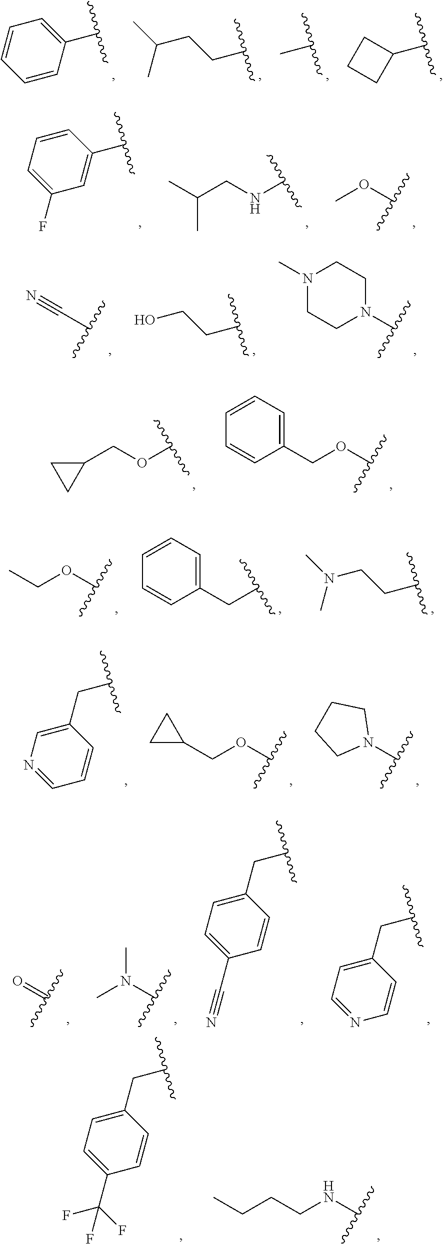

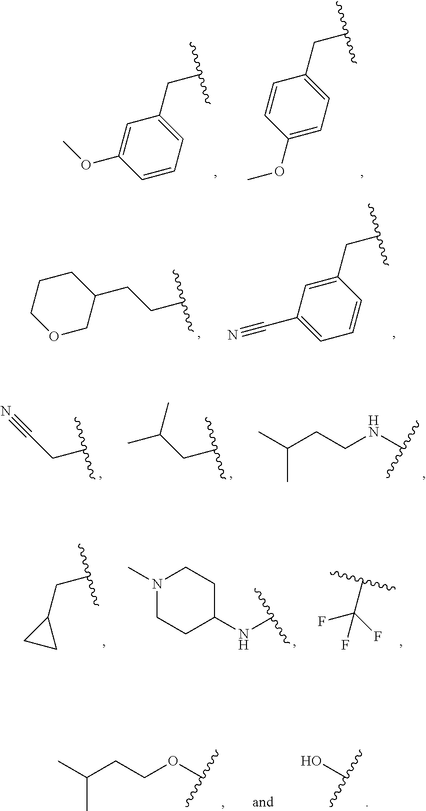

In embodiments, R⁷ is independently selected from

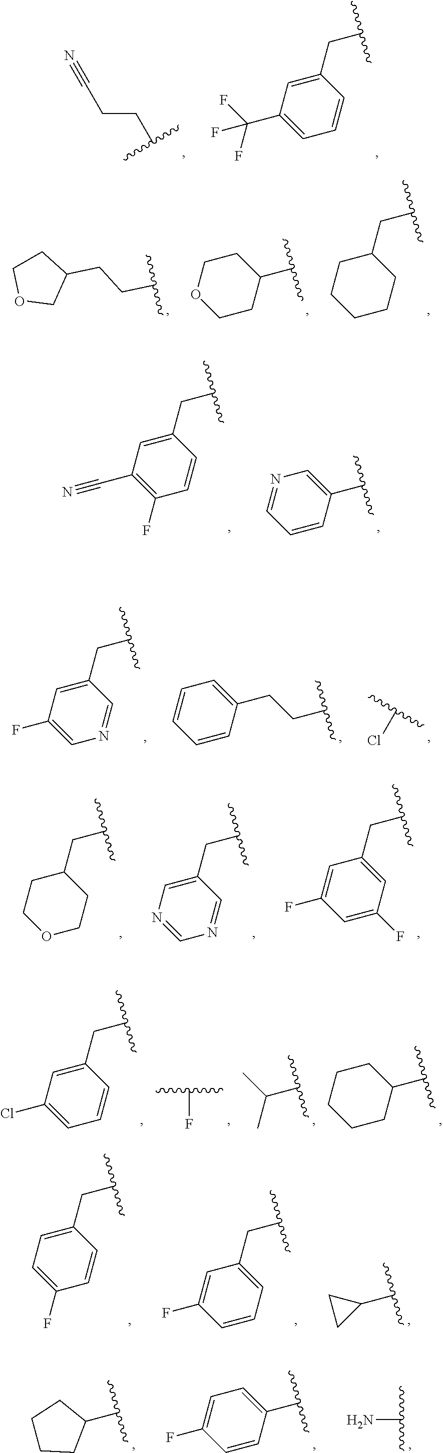

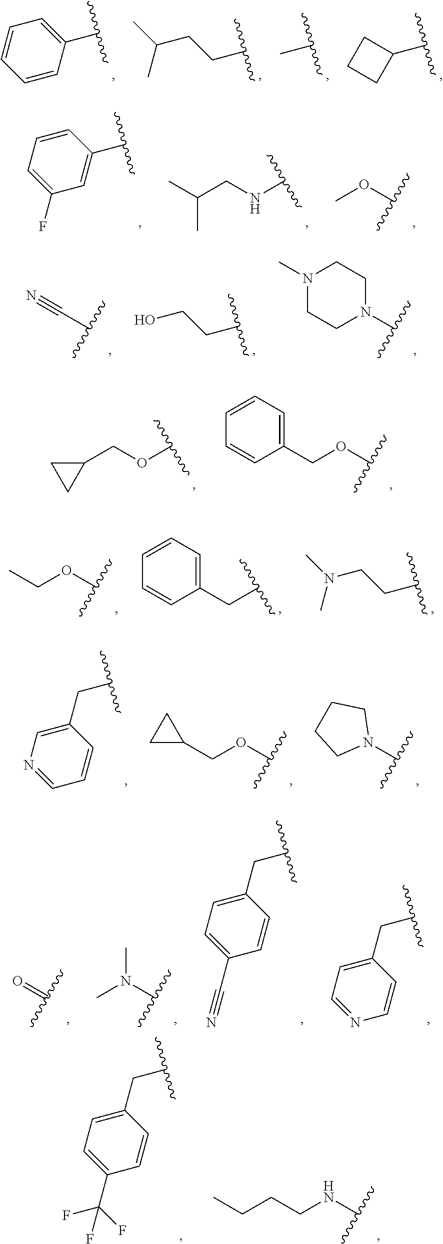

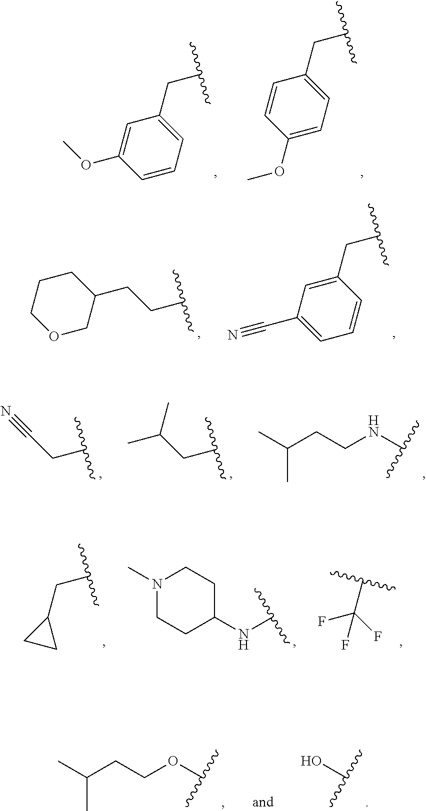

In embodiments, R^{7′} is independently selected from

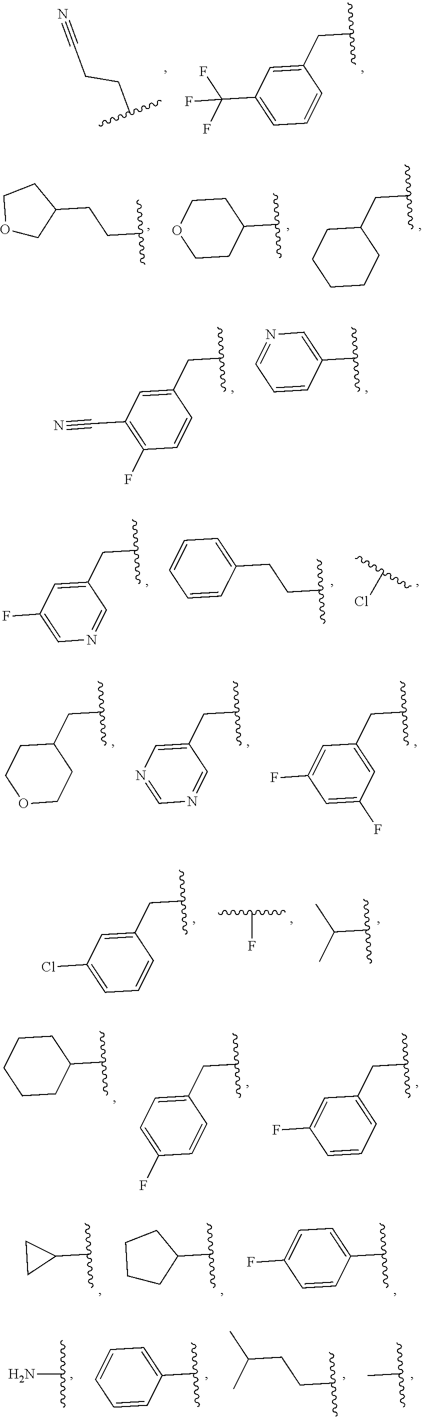

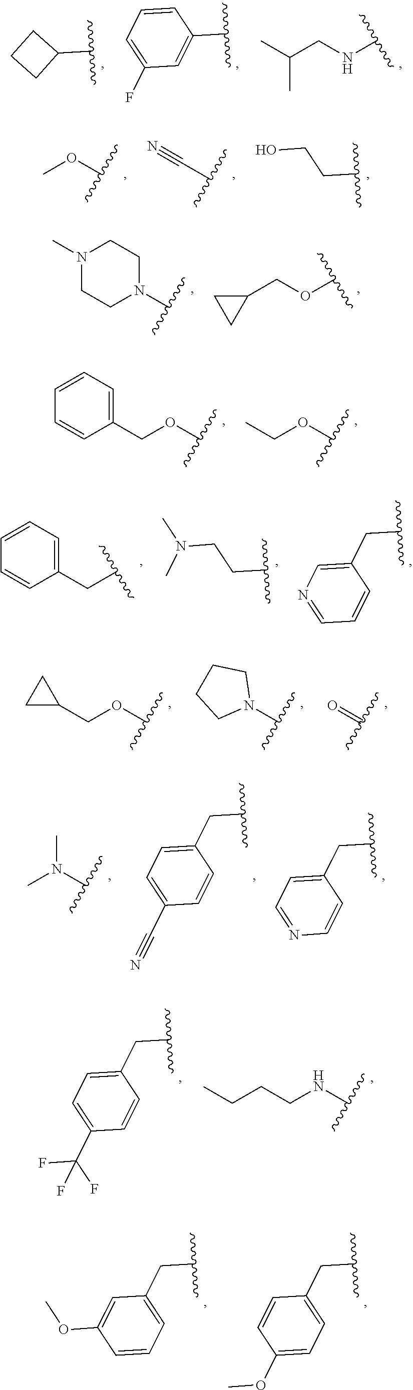

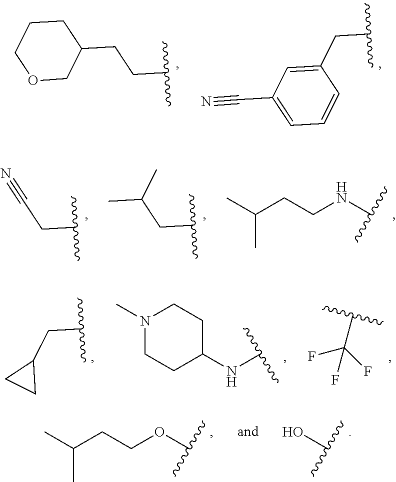

In embodiments the compound has the structure of Formula (IIa′):

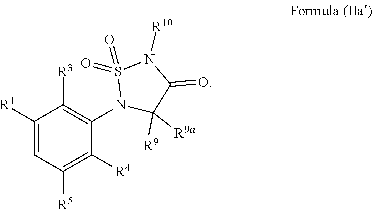

In embodiments the compound has the structure of Formula (IIb′):

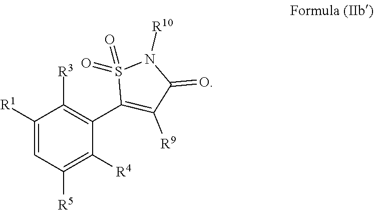

In embodiments the compound has the structure of Formula (IIc′):

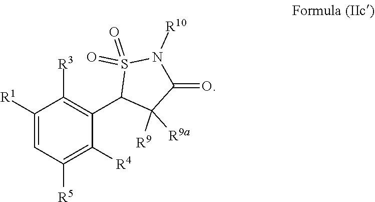

In embodiments the compound has the structure of Formula (IIa″):

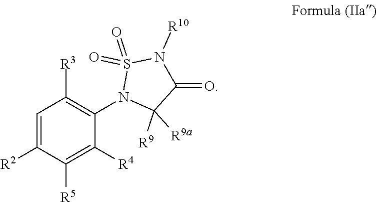

In embodiments the compound has the structure of Formula (IIIb″):

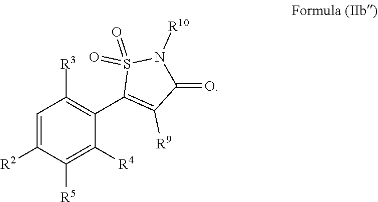

In embodiments the compound has the structure of Formula (IIc″):

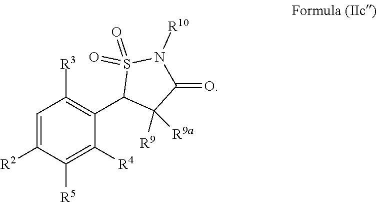

In embodiments the compound has the structure of Formula (IId):

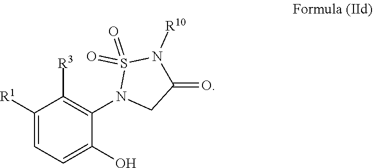

In embodiments the compound has the structure of Formula (IIe):

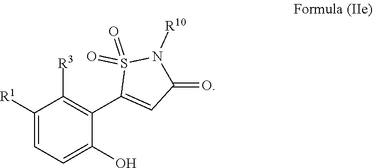

In embodiments the compound has the structure of Formula (IIf):

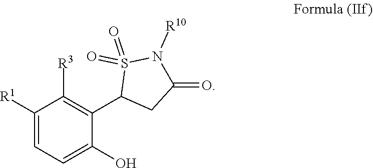

In embodiments the compound has the structure of Formula (IIg):

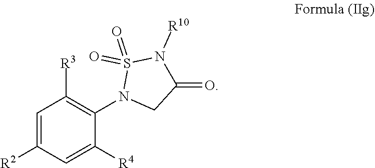

In embodiments the compound has the structure of Formula (IIh):

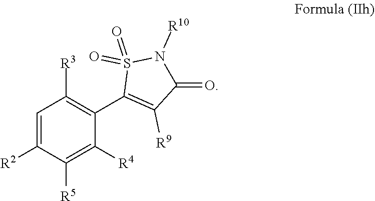

In embodiments the compound has the structure of Formula (IIi):

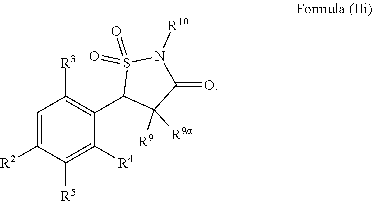

In embodiments the compound of Formula (IIa) has the structure of

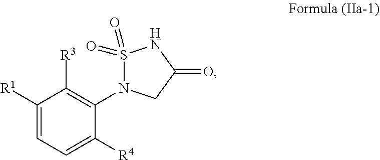

such as

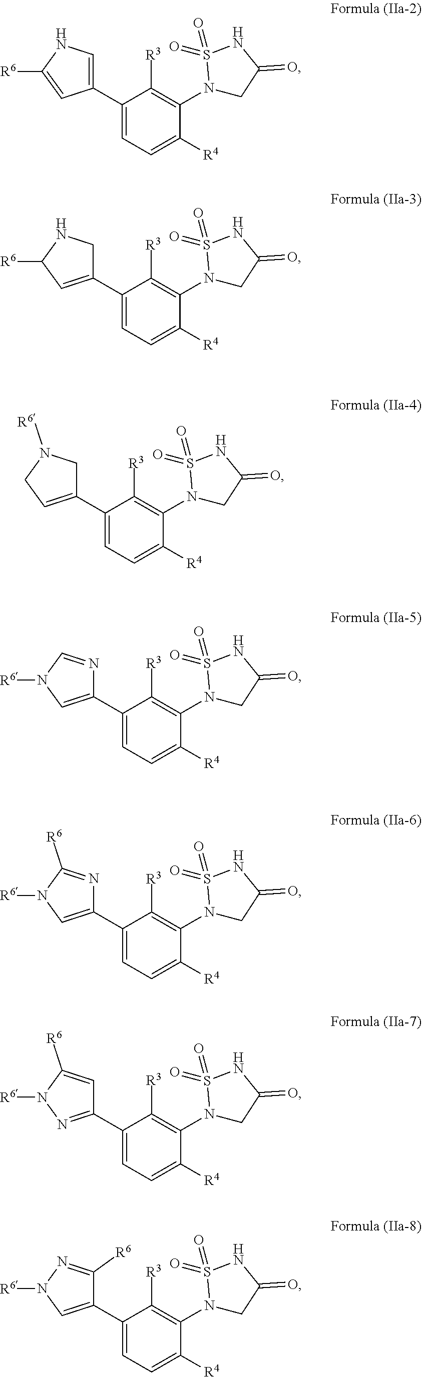

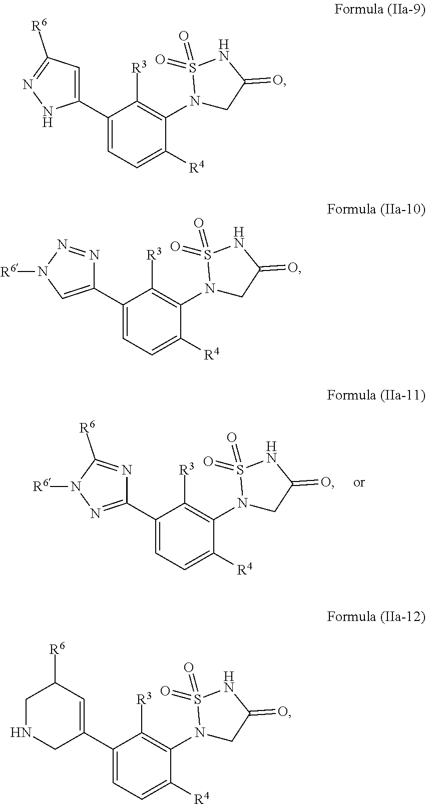

or a pharmaceutically acceptable salt or solvate thereof. In some embodiments, the compound of Formula (IIa) is a compound of Formula (IIa-1). In some embodiments, the compound of Formula (IIa) is a compound of Formula (IIa-2). In some embodiments, the compound of Formula (IIa) is a compound of Formula (IIa-3). In some embodiments, the compound of Formula (IIa) is a compound of Formula (IIa-4). In some embodiments, the compound of Formula (IIa) is a compound of Formula (IIa-5). In some embodiments, the compound of Formula (IIa) is a compound of Formula (IIa-6). In some embodiments, the compound of Formula (IIa) is a compound of Formula (IIa-7). In some embodiments, the compound of Formula (IIa) is a compound of Formula (IIa-8). In some embodiments, the compound of Formula (IIa) is a compound of Formula (IIa-9). In some embodiments, the compound of Formula (IIa) is a compound of Formula (IIa-10). In some embodiments, the compound of Formula (IIa) is a compound of Formula (IIa-11). In some embodiments, the compound of Formula (IIa) is a compound of Formula (IIa-12).

In some embodiments, for a compound of Formula (IIa-1), R¹ is selected from C₅₋₈cycloalkyl, 5 to 8 membered heterocycloalkyl, C₆aryl, and 5 to 6 membered heteroaryl, wherein C₅₋₈cycloalkyl, 5 to 8 membered heterocycloalkyl, C₆aryl, and 5 to 6 membered heteroaryl are substituted with one or more substituents independently selected from R⁶ and R^{6′}, wherein ring heteroatoms of the 5 to 8 membered heterocycloalkyl and 5 to 6 membered heteroaryl are independently selected from N, O, and S. In some embodiments, the C₅₋₈cycloalkyl is saturated. In some embodiments, the C₅₋₈cycloalkyl is partially unsaturated. In some embodiments, the C₅₋₈cycloalkyl comprises one double bond. In embodiments, the 5 to 8 membered heterocycloalkyl is saturated. In some embodiments, the 5 to 8 membered heterocycloalkyl is partially unsaturated. In some embodiments, the 5 to 8 membered heterocycloalkyl comprises one double bond. In some embodiments, R¹ is selected from pyrrolyl, pyrazolyl, imidazolyl, triazolyl, pyrrolinyl, pyridinyl, tetrahydropyridinyl, piperidinyl, and piperazinyl, each of which is optionally substituted with one or more substituents independently selected from R⁶ and R^{6′}. In some embodiments, R¹ is selected from pyrrolyl, pyrazolyl, imidazolyl, triazolyl, pyrrolinyl, pyridinyl, tetrahydropyridinyl, piperidinyl, and piperazinyl, each of which is optionally substituted with one or more substituents independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, —OR¹², —N(R¹²)(R¹³), —C(O)OR¹², —C(NR¹⁴)N(R¹²)(R¹³), —N(R¹⁴)S(O)₂R¹⁵, —C(O)R¹⁵, —C(O)N(R¹²)(R¹³), —N(R¹⁴)C(O)R¹⁵, —S(O)₂R¹⁵, and —S(O)₂N(R¹²)(R¹³)—, wherein C₁₋₆alkyl, C₂₋₆alkenyl, C₂₋₆alkynyl, C₃₋₁₀cycloalkyl, C₂₋₉heterocycloalkyl, and C₆₋₁₀aryl are optionally substituted with one, two, or three R²⁶. In some embodiments, each R¹³ is independently selected from hydrogen, —CN, C₁₋₆ alkyl, and C₁₋₆haloalkyl; or R¹² and R¹³, together with the nitrogen to which they are attached, form a C₂₋₉heterocycloalkyl ring optionally substituted with one, two, or three R^{20l}. In some embodiments, R¹ is substituted with —C(NR¹⁴)N(R¹²)(R¹³), such as —C(NH)NH₂ or —C(NH)NHCN.

In some embodiments, for a compound of Formula (IIa-1), (IIa-2), (IIa-3), (IIa-4), (IIa-5), (IIa-6), (IIa-7), (IIa-8), (IIa-9), (IIa-10), (IIa-11), or (IIa-12), R³ is halogen, such as chloro or fluoro. In some embodiments, R³ is fluoro. In some embodiments, R⁴ is selected from halogen, —OH, and C₁₋₃ haloalkyl. In some embodiments, R⁴ is selected from halogen, —OH, and —CHF₂. In embodiments, R⁴ is —OH.

In some embodiments, for a compound of Formula (IIa-1), (IIa-2), (IIa-3), (IIa-4), (IIa-5), (IIa-6), (IIa-7), (IIa-8), (IIa-9), (IIa-10), (IIa-11), or (IIa-12), R⁶ is selected from halogen, oxo, —CN, C₁₋₆alkyl, C₂₋₆alkenyl, C₂₋₆alkynyl, C₃₋₁₀cycloalkyl, C₂₋₉heterocycloalkyl, —OR¹², —N(R¹²)(R¹³), —C(O)OR¹², —N(R¹⁴)S(O)₂R¹⁵, —C(O)R¹⁵, —OC(O)R¹⁵, —C(O)N(R¹²)(R¹³), —N(R¹⁴)C(O)R¹⁵, —S(O)₂R¹⁵, —S(O)₂N(R¹²)(R¹³)—, —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₁₀cycloalkyl, and C₂₋₉heterocycloalkyl are optionally substituted with one, two, or three R²⁶; and R^{6′} is selected from —CN, C₁₋₆alkyl, C₂₋₆alkenyl, C₂₋₆alkynyl, C₃₋₁₀cycloalkyl, C₂₋₉heterocycloalkyl, —OR¹², —C(O)OR¹², —C(O)R¹⁵, —OC(O)R¹⁵, —C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₁₀cycloalkyl, and C₂₋₉heterocycloalkyl are optionally substituted with one, two, or three R²⁶. In some embodiments, R⁶ is selected from halogen, C₁₋₆alkyl, C₂₋₆alkenyl, C₃₋₁₀cycloalkyl, —OR¹², and —N(R¹²)(R¹³), wherein C₁₋₆alkyl, C₂₋₆alkenyl, and C₃₋₁₀cycloalkyl are optionally substituted with one, two, or three R²⁶; and R^{6′} is selected from C₁₋₆alkyl, C₂₋₆alkenyl, C₃₋₁₀cycloalkyl, —C(O)OR¹², —C(O)R¹⁵, and —C(O)N(R¹²)(R¹³), wherein C₁₋₆alkyl, C₂₋₆alkenyl, and C₃₋₁₀cycloalkyl are optionally substituted with one, two, or three R²⁶. In some embodiments, R²⁶ is independently selected from halogen, —CN, C₁₋₆alkyl, C₁₋₆ haloalkyl, C₂₋₆alkenyl, C₃₋₁₀cycloalkyl, C₆aryl, —OR¹², —N(R¹²)(R¹³), —C(O)OR¹², —C(O)R¹⁵, —OC(O)R¹⁵, —C(O)N(R¹²)(R¹³), and —N(R¹⁴)C(O)R¹⁵, wherein C₁₋₆alkyl, C₁₋₆haloalkyl, C₂₋₆alkenyl, C₃₋₁₀cycloalkyl, and C₆aryl are optionally substituted with one, two, or three R²⁷; and each R²⁷ is independently selected from oxo, halogen, —CN, C₁₋₆alkyl, C₂₋₆alkenyl, —OR¹², —N(R¹²)(R¹³), —C(O)OR¹², —C(O)R¹⁵, —OC(O)R¹⁵, —C(O)N(R¹²)(R¹³), and —N(R¹⁴)C(O)R¹⁵.

In some embodiments, for a compound of Formula (IIa-1), (IIa-2), (IIa-3), (IIa-4), (IIa-5), (IIa-6), (IIa-7), (IIa-8), (IIa-9), (IIa-10), (IIa-11), or (IIa-12), R⁶ is selected from halogen, C₁₋₆alkyl, C₁₋₆haloalkyl, C₃₋₆cycloalkyl, —OH, —OCH₃, and —NH₂, wherein C₁₋₆ alkyl and C₃₋₆cycloalkyl are optionally substituted with one, two, or three substituents selected from halogen, —CN, C₁₋₆haloalkyl, —(C₁₋₆alkyl)OH, C₃₋₆cycloalkyl, C₆aryl, —O(C₁₋₆alkyl), —O(C₁₋₆haloalkyl), and —C(O)NH₂. In some embodiments, R⁶ is selected from fluoro, chloro, C₁₋₆alkyl, C₁₋₆fluoroalkyl, C₃₋₆cycloalkyl, —OH, —OCH₃, and —NH₂, wherein C₁₋₆alkyl and C₃₋₆cycloalkyl are optionally substituted with one, two, or three substituents selected from fluoro, chloro, —CN, C₁₋₆fluoroalkyl, —CH₂OH, C₃₋₆cycloalkyl, —OCH₃, —OCH₂F, —OCHF₂, —OCF₃, and —C(O)NH₂. In some embodiments, R⁶ is fluoro. In some embodiments, R⁶ is C₁₋₆ alkyl. In some embodiments, R⁶ is C₁₋₆fluoroalkyl. In some embodiments, R⁶ is C₃₋₆cycloalkyl. In some embodiments, R⁶ is —OH. In some embodiments, R⁶ is —OCH₃. In some embodiments, R⁶ is —NH₂.

In some embodiments, for a compound of Formula (IIa-1), (IIa-2), (IIa-3), (IIa-4), (IIa-5), (IIa-6), (IIa-7), (IIa-8), (IIa-9), (IIa-10), (IIa-11), or (IIa-12), R^{6′} is selected from C₁₋₆alkyl, C₁₋₆haloalkyl, C₃₋₆cycloalkyl, —C(O)O(C₁₋₆alkyl), —C(O)(C₃₋₆cycloalkyl), and —SO₂(C₁₋₆alkyl), wherein C₁₋₆alkyl and C₃₋₆cycloalkyl are optionally substituted with one, two, or three substituents selected from halogen, —CN, C₁₋₆haloalkyl, —(C₁₋₆alkyl)OH, C₃₋₆cycloalkyl, C₆aryl, —O(C₁₋₆alkyl), —O(C₁₋₆haloalkyl), and —C(O)NH₂. In some embodiments, R^{6′} is selected from C₁₋₆alkyl, C₁₋₆fluoroalkyl, and C₃₋₆cycloalkyl, wherein C₁₋₆alkyl and C₃₋₆cycloalkyl are optionally substituted with one, two, or three substituents selected from fluoro, chloro, —CN, and C₁₋₆haloalkyl. In some embodiments, R^{6′} is C₁₋₆ alkyl. In some embodiments, R^{6′} is C₁₋₆haloalkyl. In some embodiments, R^{6′} is C₃₋₆cycloalkyl. In some embodiments, R^{6′} is —C(O)O(C₁₋₆alkyl). In some embodiments, R^{6′} is —C(O)(C₃₋₆cycloalkyl). In some embodiments, R^{6′} is —SO₂(C₁₋₆alkyl). In some embodiments, R^{6′} is —C(NR¹⁴)N(R¹²)(R¹³), such as —C(NH)NH₂ or —C(NH)NHCN.

In some embodiments, for a compound of Formula (IIa-1), (IIa-2), (IIa-3), (IIa-4), (IIa-5), (IIa-6), (IIa-7), (IIa-8), (IIa-9), (IIa-10), (IIa-11), or (IIa-12), R³ is halogen; R⁴ is selected from halogen, —OH, and C₁₋₃ haloalkyl; R⁶ is selected from halogen, C₁₋₆alkyl, C₁₋₆haloalkyl, C₃₋₆cycloalkyl, —OH, —OCH₃, and —NH₂, wherein C₁₋₆alkyl and C₃₋₆cycloalkyl are optionally substituted with one, two, or three substituents selected from halogen, —CN, C₁₋₆haloalkyl, —(C₁₋₆alkyl)OH, C₃₋₆cycloalkyl, C₆aryl, —O(C₁₋₆alkyl), —O(C₁₋₆haloalkyl), and —C(O)NH₂; and R^{6′} is selected from C₁₋₆alkyl, C₁₋₆haloalkyl, C₃₋₆cycloalkyl, —C(O)O(C₁₋₆alkyl), —C(O)(C₃₋₆cycloalkyl), and —SO₂(C₁₋₆alkyl), wherein C₁₋₆alkyl and C₃₋₆cycloalkyl are optionally substituted with one, two, or three substituents selected from halogen, —CN, C₁₋₆haloalkyl, —(C₁₋₆alkyl)OH, C₃₋₆cycloalkyl, C₆aryl, —O(C₁₋₆alkyl), —O(C₁₋₆haloalkyl), and —C(O)NH₂. In some embodiments, R³ is fluoro; R⁴ is —OH; R⁶ is selected from halogen, C₁₋₆alkyl, C₁₋₆haloalkyl, C₃₋₆cycloalkyl, —OH, —OCH₃, and —NH₂, wherein C₁₋₆alkyl and C₃₋₆cycloalkyl are optionally substituted with one, two, or three substituents selected from halogen, —CN, C₁₋₆ haloalkyl, —(C₁₋₆alkyl)OH, C₃₋₆cycloalkyl, C₆aryl, —O(C₁₋₆alkyl), —O(C₁₋₆haloalkyl), and —C(O)NH₂; and R^{6′} is selected from C₁₋₆alkyl, C₁₋₆haloalkyl, C₃₋₆cycloalkyl, —C(O)O(C₁₋₆alkyl), —C(O)(C₃₋₆cycloalkyl), and —SO₂(C₁₋₆alkyl), wherein C₁₋₆alkyl and C₃₋₆cycloalkyl are optionally substituted with one, two, or three substituents selected from halogen, —CN, C₁₋₆haloalkyl, —(C₁₋₆alkyl)OH, C₃₋₆cycloalkyl, C₆aryl, —O(C₁₋₆alkyl), —O(C₁₋₆haloalkyl), and —C(O)NH₂.

In embodiments the compound has the structure of Formula (IId′); OH Formula (IId′).

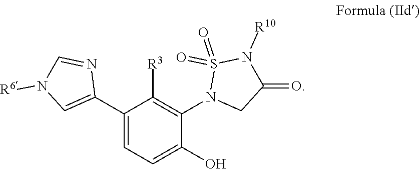

In embodiments the compound has the structure of Formula (IId″):

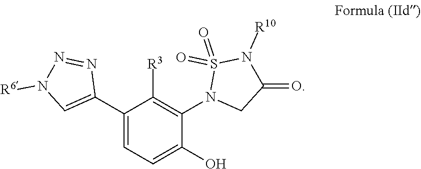

In embodiments of formula IId′, R³ is F. In embodiments of formula IId′, R³ is C₁. In embodiments of formula IId′, R¹⁰ is H. In embodiments of formula IId″, R³ is F. In embodiments of formula IId″, R³ is C₁. In embodiments of formula IId″, R¹⁰ is H. In embodiments of formula IId′, R^{6′} is —CH₂-phenyl-(R²⁷)₀₋₃. In embodiments of formula IId″, R^{6′} is —CH₂-phenyl-(R²⁷)₀₋₃. In embodiments, R²⁷ is independently halogen. In embodiments, R²⁷ is independently —CN. In embodiments, R²⁷ is independently methyl. In embodiments, R²⁷ is independently ethyl. In embodiments, R²⁷ is independently CF₃. In embodiments, R²⁷ is independently —NH₂.

In an aspect is provided a compound of Formula (VIII), or a pharmaceutically acceptable salt or solvate thereof:

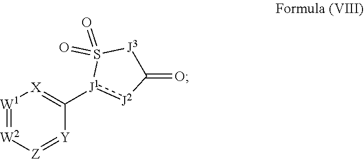

wherein:
either: W¹ is C(R¹), and W² is C(R^{2a}) or N; or W¹ is C(R^{1a}) or N, and W² is C(R²);
R¹ is selected from C₅₋₁₂cycloalkyl, 5 to 12 membered heterocycloalkyl, and 5 to 12 membered heteroaryl, wherein C₅₋₁₂cycloalkyl, 5 to 12 membered heterocycloalkyl, C₆₋₁₂aryl, and 5 to 12 membered heteroaryl are substituted with one or more substituents independently selected from R⁶ and R^{6′}, wherein ring heteroatoms of the 5 to 12 membered heterocycloalkyl and 5 to 12 membered heteroaryl are independently selected from N, O, and S;
R^{1a} is selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20a};
R² is selected from C₅₋₁₂cycloalkyl, 5 to 12 membered heterocycloalkyl, and 5 to 12 membered heteroaryl, wherein C₅₋₁₂cycloalkyl, 5 to 12 membered heterocycloalkyl, C₆₋₁₂aryl, and 5 to 12 membered heteroaryl are substituted with one or more substituents independently selected from R⁷ and R^{7′}, wherein ring heteroatoms of the 5 to 12 membered heterocycloalkyl and 5 to 12 membered heteroaryl are independently selected from N, O, and S;
R^{2a} are independently selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20b};
X is N or C(R³);
Y is N or C(R⁴);
Z is N or C(R⁵);
R³ is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20c};
R⁴ is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20e};
each R⁶ and R⁷ are independently selected from halogen, oxo, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁶;
each R^{6′} and R^{7′} are independently selected from —CN, C₁₋₆alkyl, C₂₋₆alkenyl, C₂₋₆alkynyl, C₃₋₁₀cycloalkyl, C₂₋₉heterocycloalkyl, C₆₋₁₀ aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶;
J^{P} is N, C, or C(R¹);
J² is N, N(R⁹), C(R⁹), C(R⁹)(R^{9a}), or C(O);
J³ is N(R¹⁰) or C(R¹⁰)(R^{10a});
R⁸ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20h};
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁰ⁱ; or R⁹ and R^{9a} are combined to form a C₃₋₆cycloalkyl or a C₂₋₉ heterocycloalkyl, wherein the C₃₋₆cycloalkyl and C₂₋₉heterocycloalkyl are optionally substituted with one, two, or three R²⁰ⁱ;
R¹⁰ and R^{10a} are independently selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R^{20j}; or R¹⁰ and R^{10a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R^{20j};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉ heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉ heteroaryl are optionally substituted with one, two, or three R^{20k};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20l};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted with one, two, or three R^{20m};
each R¹⁶ is independently selected from —C₁₋₆alkylene-OP(O)(OR^{16a})(OR^{16b}) and —P(O)(OR^{16a})(OR^{16b}); wherein the C₁₋₆alkylene is optionally substituted with one, two, or three R²⁰ⁿ; or —C₁₋₆alkylene-OC(O)—R^{16c} wherein C₁₋₆alkylene is optionally substituted with one, two, or three R²⁰ⁿ;
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20o};
R^{16c} is halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —CH₂—C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R⁴)C(O)N(R²²)(R²³), —N(R²)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three of R^{20o};
each R^{20a}, R^{20b}, R^{20c}, R^{20d}, R^{20e}, R^{20f}, R^{20h}, R²⁰ⁱ, R^{20j}, R^{20k}, R^{20l}, R^{20m}, R²⁰ⁿ, and R^{20o} are each independently selected from oxo, —CN, halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl optionally substituted with one or more R²⁶, C₁₋₆haloalkyl, C₁₋₆alkoxy, C₁₋₉haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉ heteroaryl;
each R²⁶ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁷;
each R²⁷ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³)—, S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R^{20f};
[symbol] indicates a single or double bond such that all valences are satisfied.

In an aspect is provided a compound selected from

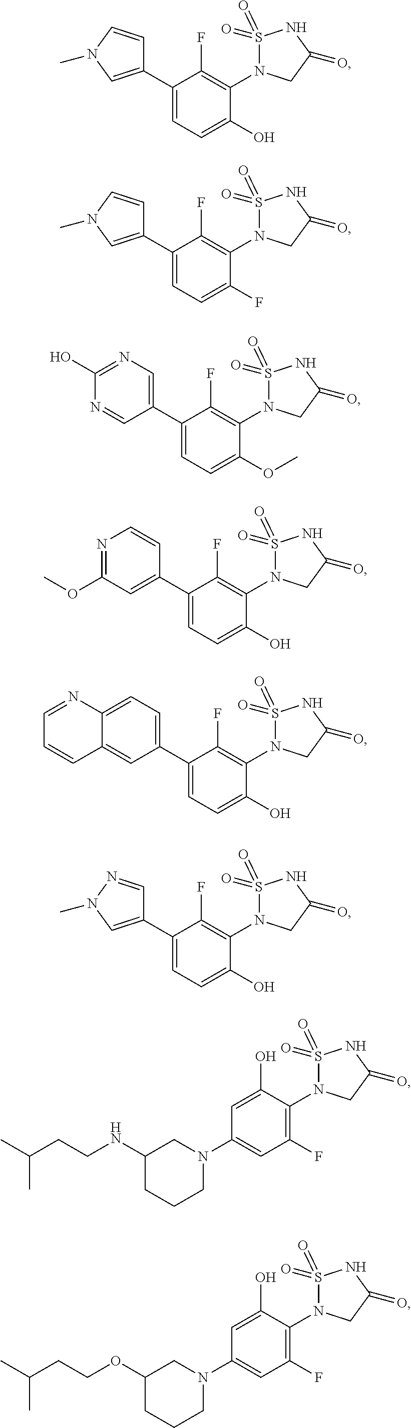

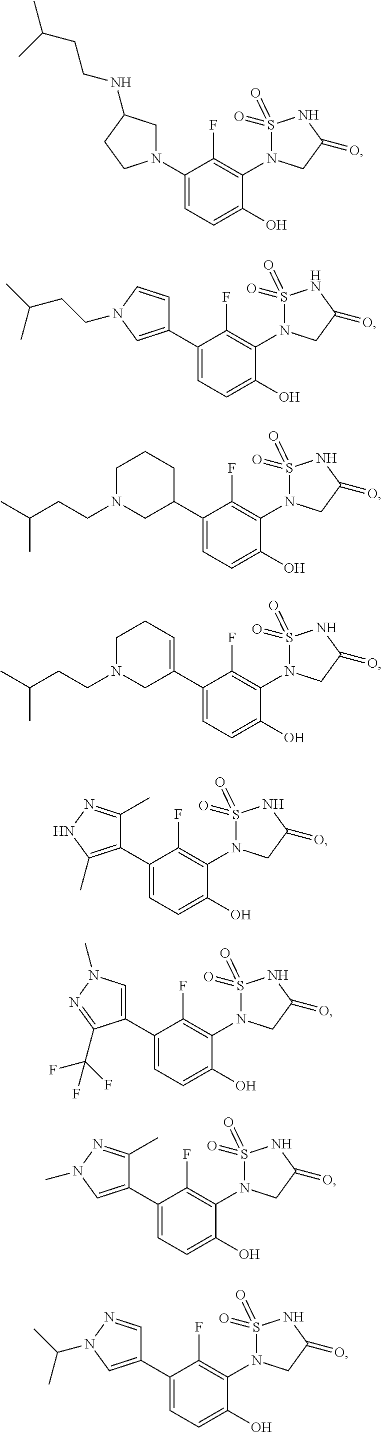

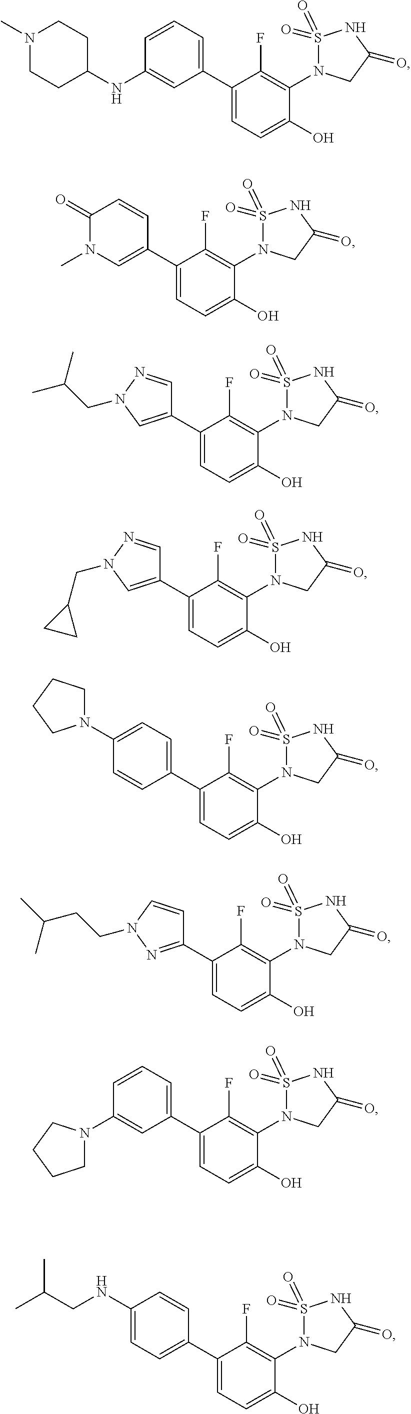

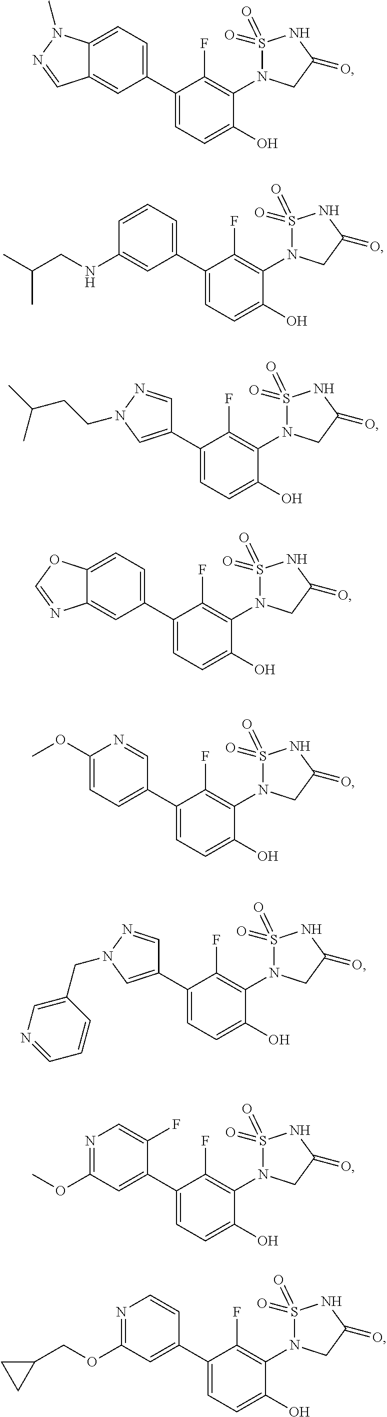

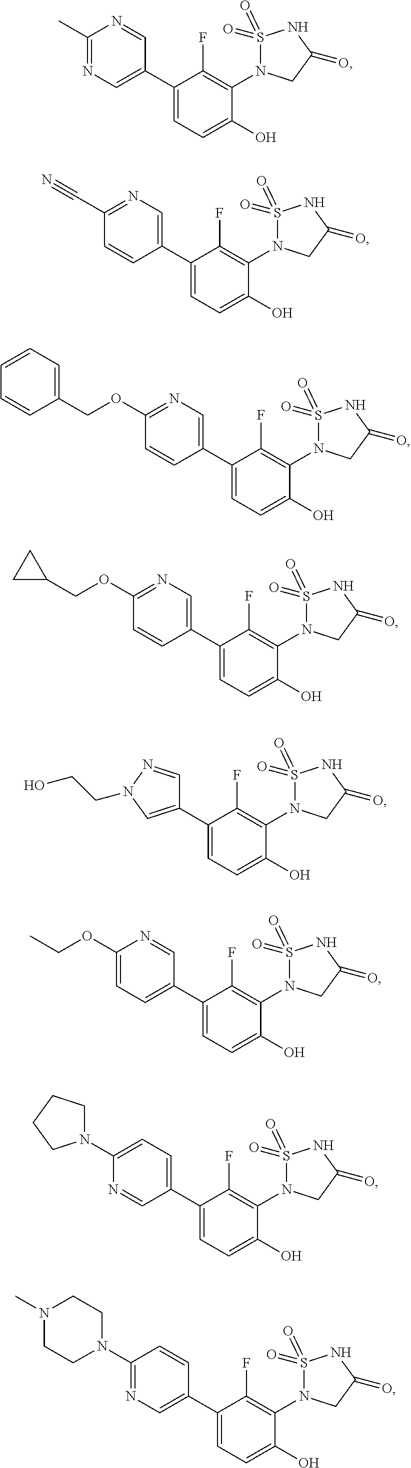

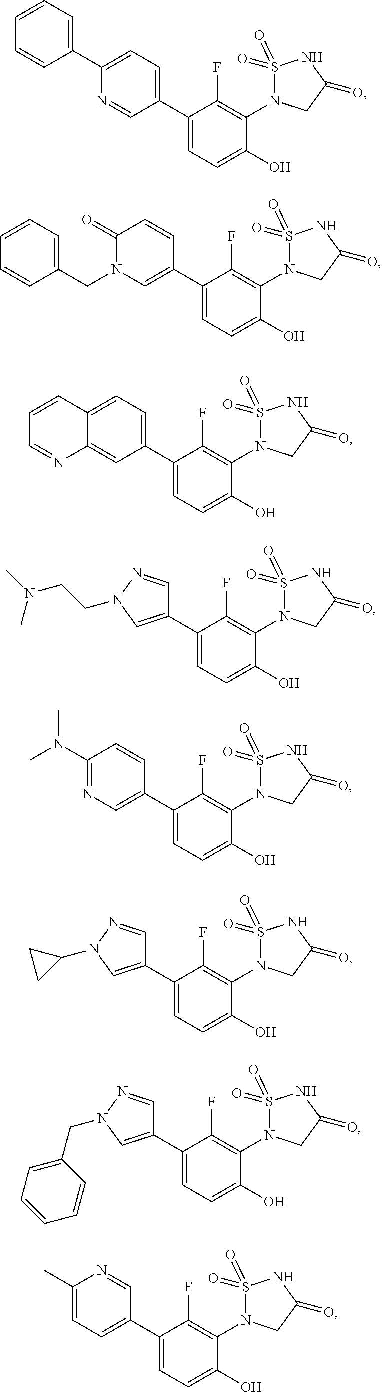

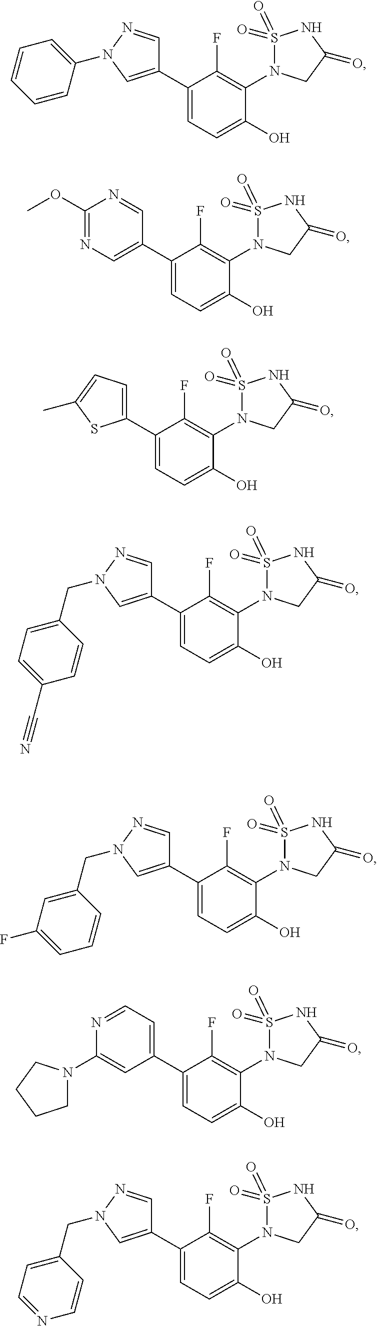

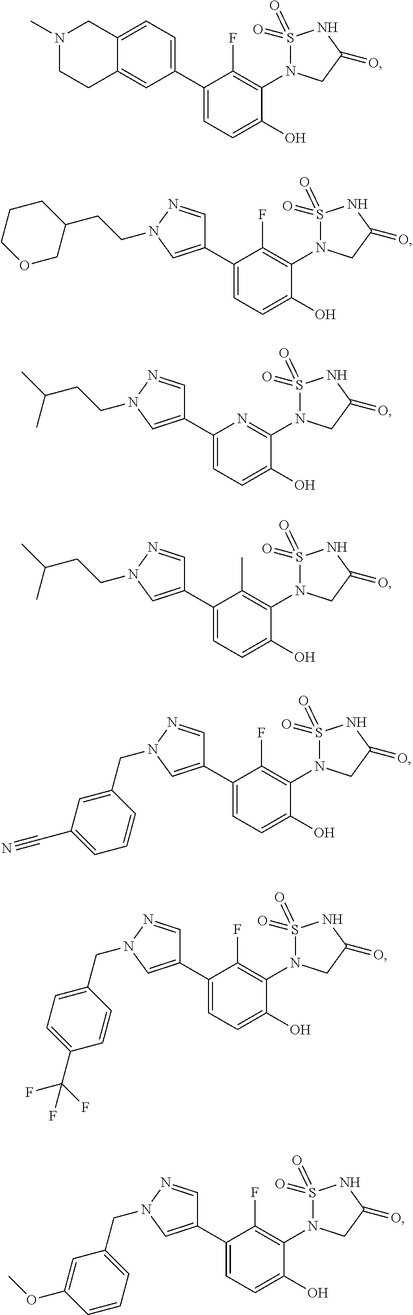

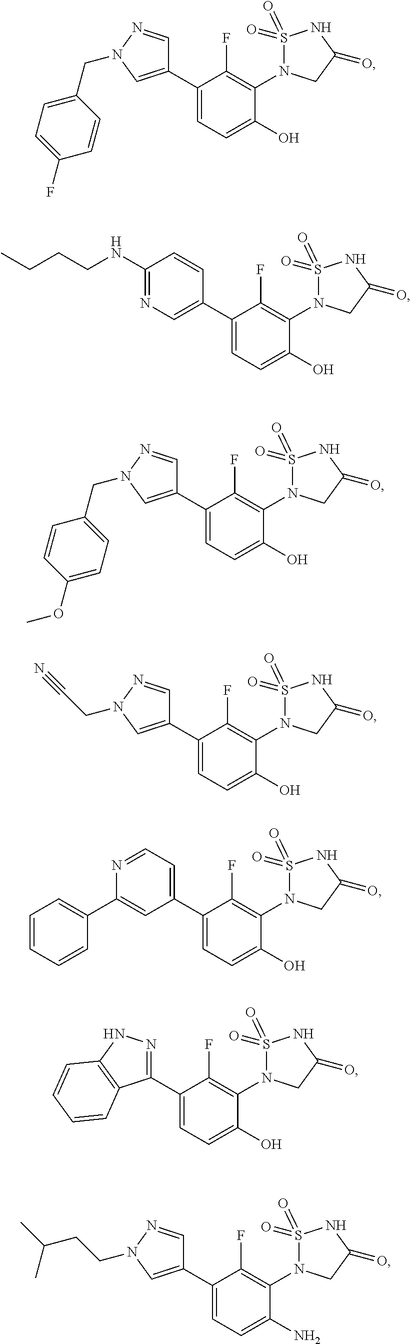

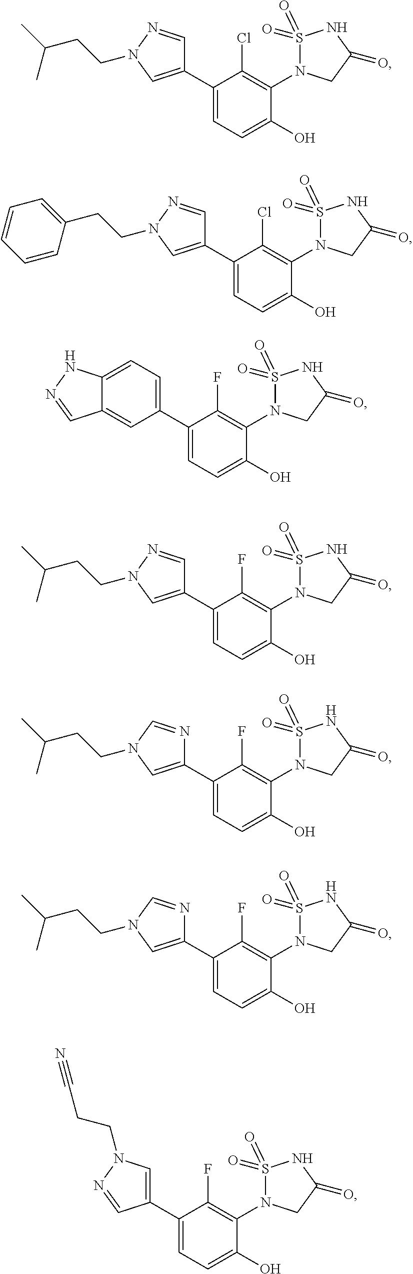

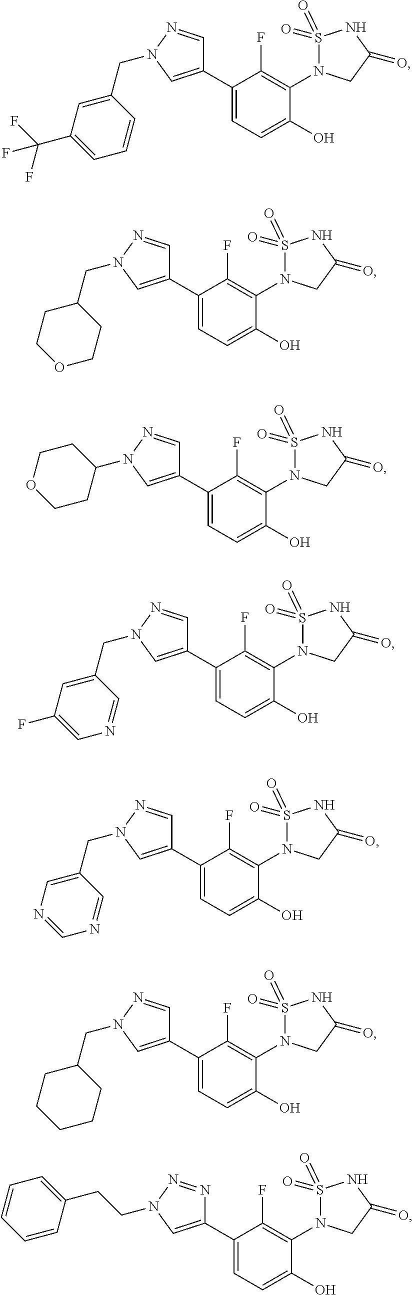

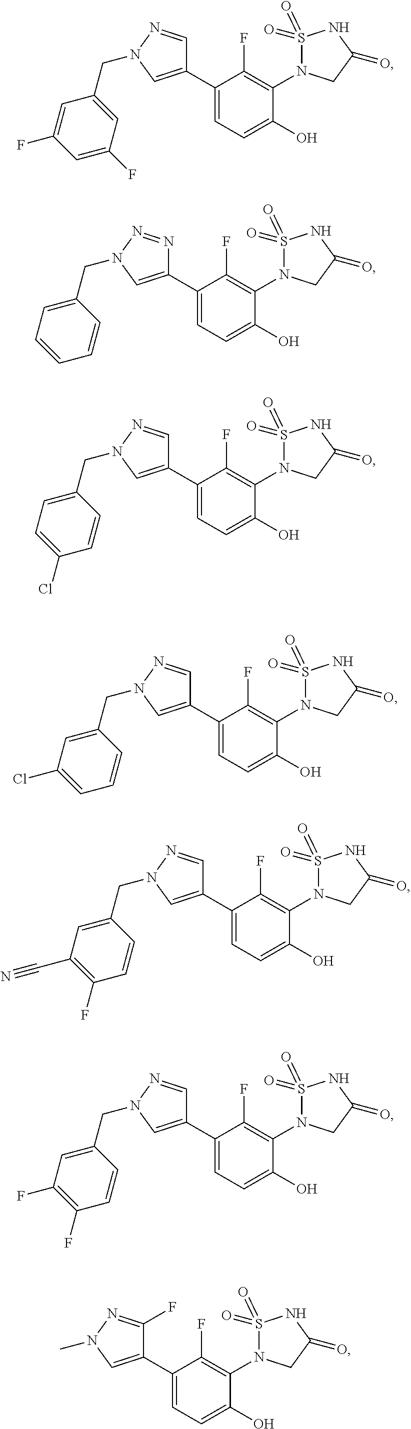

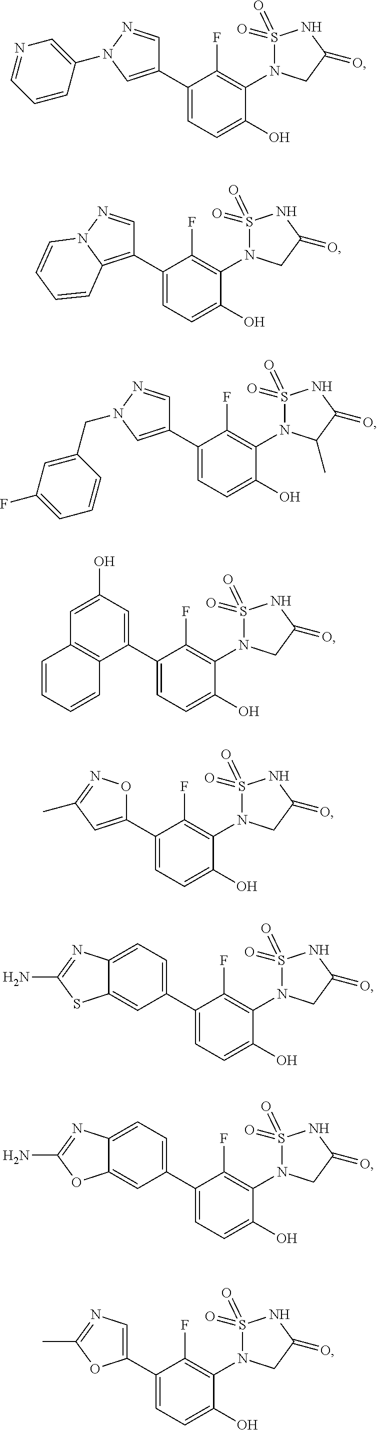

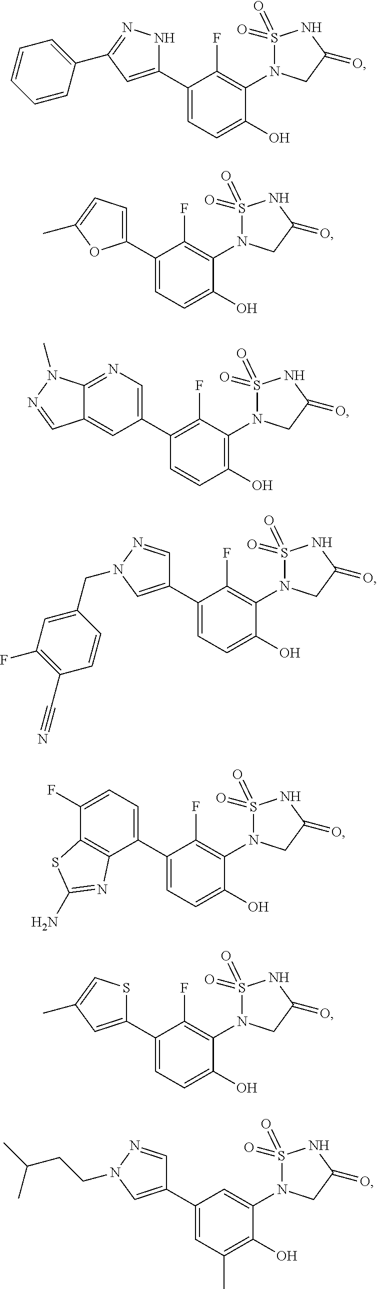

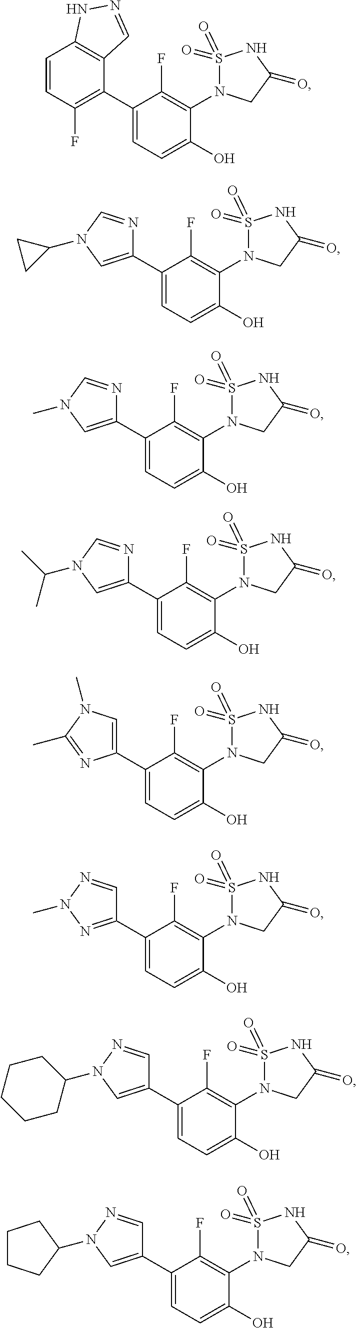

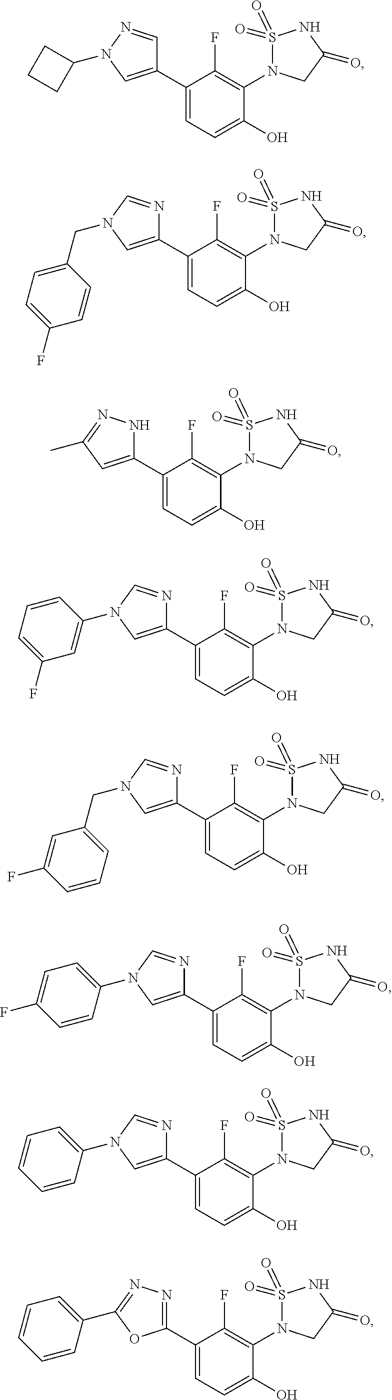

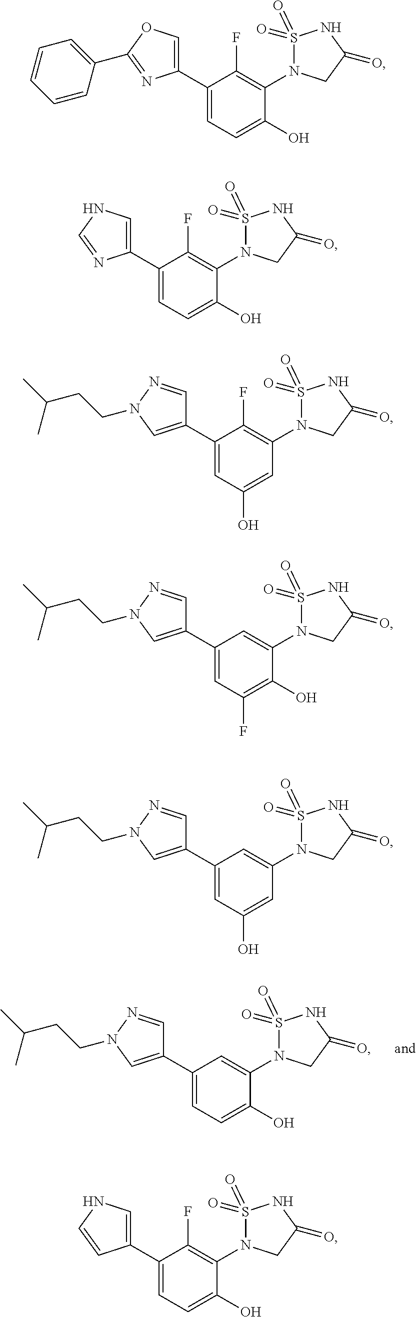

or a pharmaceutically acceptable salt or solvate thereof.

In an aspect is provided a compound selected from

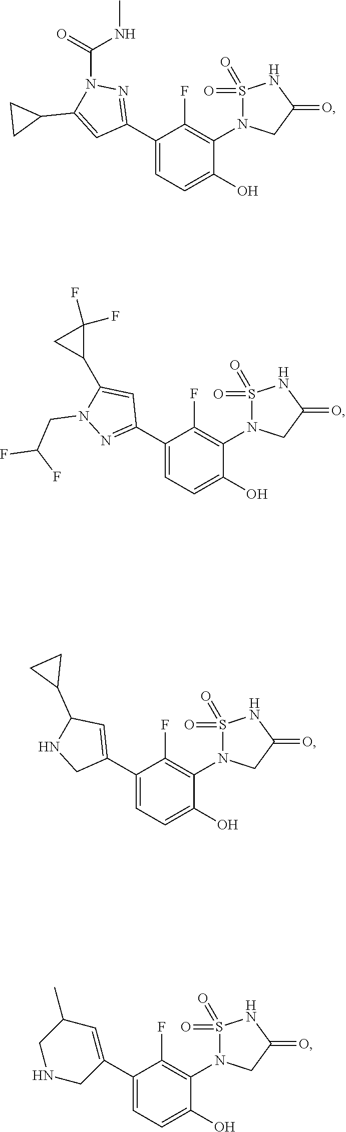 [SMILES: CNC(=O)n1nc(-c2ccc(O)c(N3CC(=O)NS3(=O)=O)c2F)cc1C1CC1 ; O=C1CN(c2c(O)ccc(-c3cc(C4CC4(F)F)n(CC(F)F)n3)c2F)S(=O)(=O)N1 ; O=C1CN(c2c(O)ccc(C3=CC(C4CC4)NC3)c2F)S(=O)(=O)N1 ; CC1C=C(c2ccc(O)c(N3CC(=O)NS3(=O)=O)c2F)CNC1]

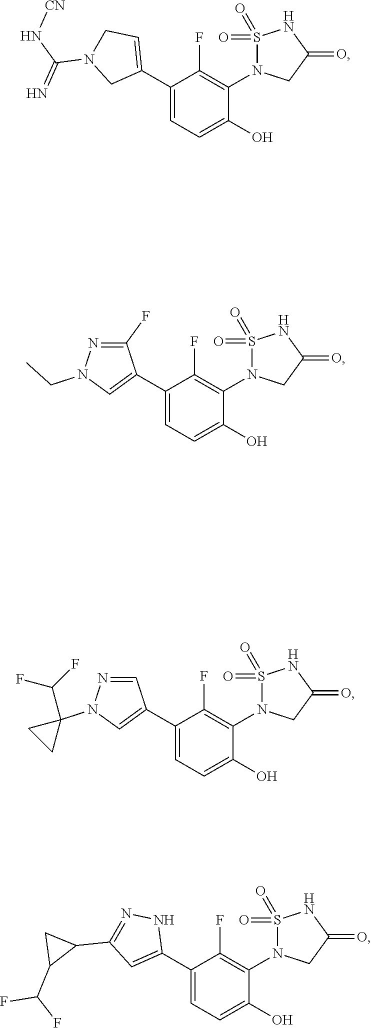 [SMILES: N#CNC(=N)N1CC=C(c2ccc(O)c(N3CC(=O)NS3(=O)=O)c2F)C1 ; CCn1cc(-c2ccc(O)c(N3CC(=O)NS3(=O)=O)c2F)c(F)n1 ; O=C1CN(c2c(O)ccc(-c3cnn(C4(C(F)F)CC4)c3)c2F)S(=O)(=O)N1 ; O=C1CN(c2c(O)ccc(-c3cc(C4CC4C(F)F)n[nH]3)c2F)S(=O)(=O)N1]

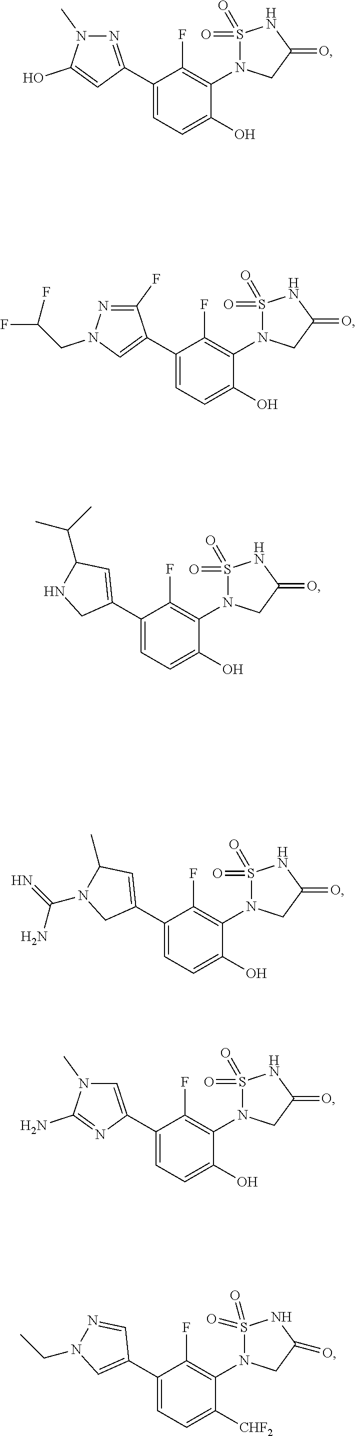 [SMILES: Cn1nc(-c2ccc(O)c(N3CC(=O)NS3(=O)=O)c2F)cc1O ; O=C1CN(c2c(O)ccc(-c3cn(CC(F)F)nc3F)c2F)S(=O)(=O)N1 ; CC(C)C1C=C(c2ccc(O)c(N3CC(=O)NS3(=O)=O)c2F)CN1 ; CC1C=C(c2ccc(O)c(N3CC(=O)NS3(=O)=O)c2F)CN1C(=N)N ; Cn1cc(-c2ccc(O)c(N3CC(=O)NS3(=O)=O)c2F)nc1N ; CCn1cc(-c2ccc(C(F)F)c(N3CC(=O)NS3(=O)=O)c2F)cn1]

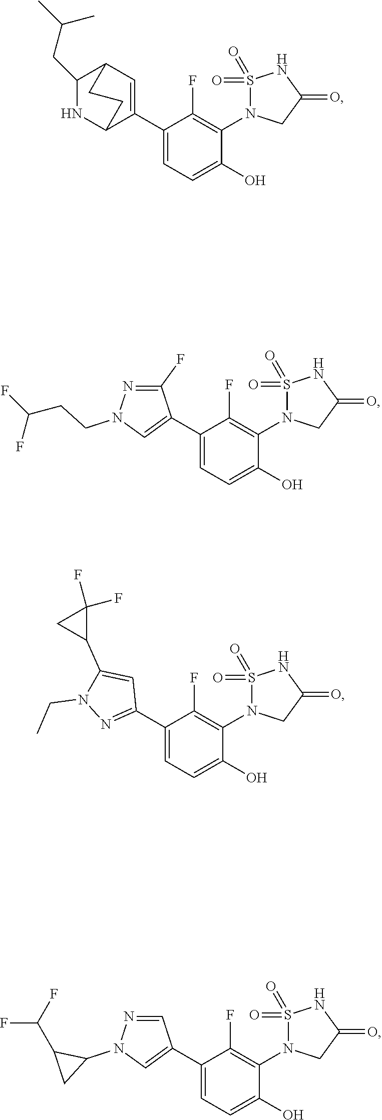 [SMILES: CC(C)CC1NC2CCC1C=C2c1ccc(O)c(N2CC(=O)NS2(=O)=O)c1F ; O=C1CN(c2c(O)ccc(-c3cn(CCC(F)F)nc3F)c2F)S(=O)(=O)N1 ; CCn1nc(-c2ccc(O)c(N3CC(=O)NS3(=O)=O)c2F)cc1C1CC1(F)F ; O=C1CN(c2c(O)ccc(-c3cnn(C4CC4C(F)F)c3)c2F)S(=O)(=O)N1]

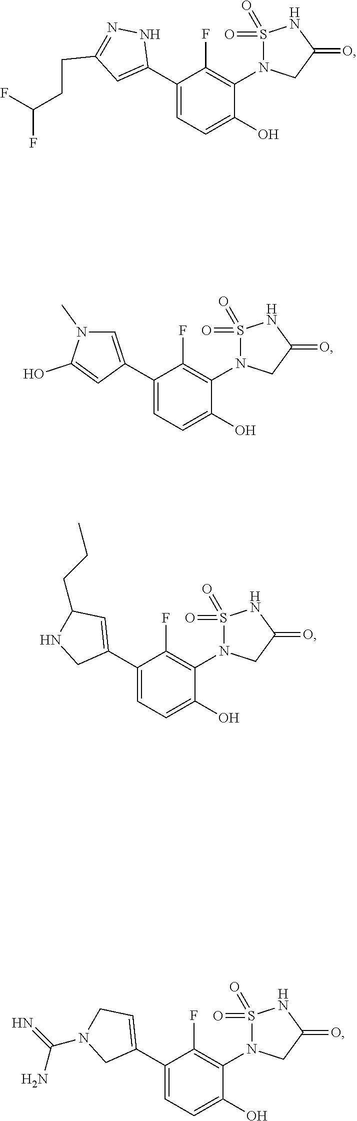 [SMILES: O=C1CN(c2c(O)ccc(-c3cc(CCC(F)F)n[nH]3)c2F)S(=O)(=O)N1 ; Cn1cc(-c2ccc(O)c(N3CC(=O)NS3(=O)=O)c2F)cc1O ; CCCC1C=C(c2ccc(O)c(N3CC(=O)NS3(=O)=O)c2F)CN1 ; N=C(N)N1CC=C(c2ccc(O)c(N3CC(=O)NS3(=O)=O)c2F)C1]

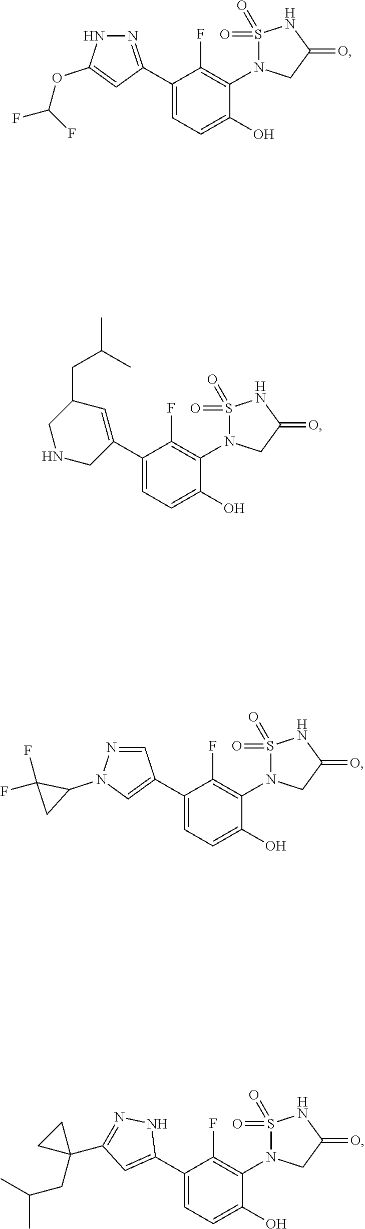 [SMILES: O=C1CN(c2c(O)ccc(-c3cc(OC(F)F)[nH]n3)c2F)S(=O)(=O)N1 ; CC(C)CC1C=C(c2ccc(O)c(N3CC(=O)NS3(=O)=O)c2F)CNC1 ; O=C1CN(c2c(O)ccc(-c3cnn(C4CC4(F)F)c3)c2F)S(=O)(=O)N1 ; CC(C)CC1(c2cc(-c3ccc(O)c(N4CC(=O)NS4(=O)=O)c3F)[nH]n2)CC1]

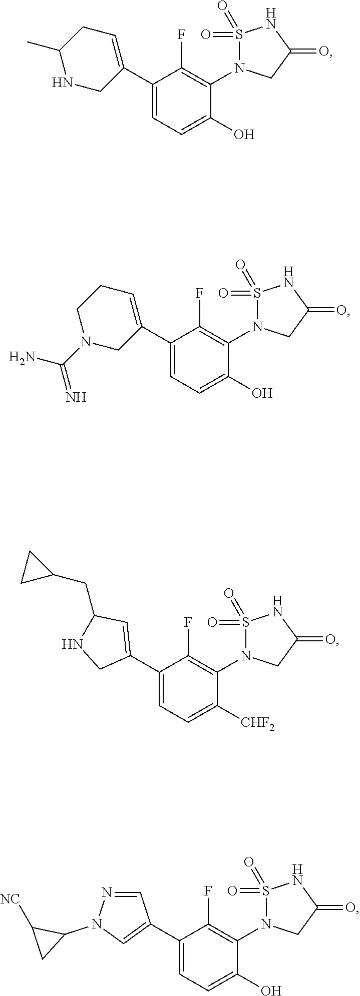 [SMILES: CC1CC=C(c2ccc(O)c(N3CC(=O)NS3(=O)=O)c2F)CN1 ; N=C(N)N1CCC=C(c2ccc(O)c(N3CC(=O)NS3(=O)=O)c2F)C1 ; O=C1CN(c2c(C(F)F)ccc(C3=CC(CC4CC4)NC3)c2F)S(=O)(=O)N1 ; N#CC1CC1n1cc(-c2ccc(O)c(N3CC(=O)NS3(=O)=O)c2F)cn1]

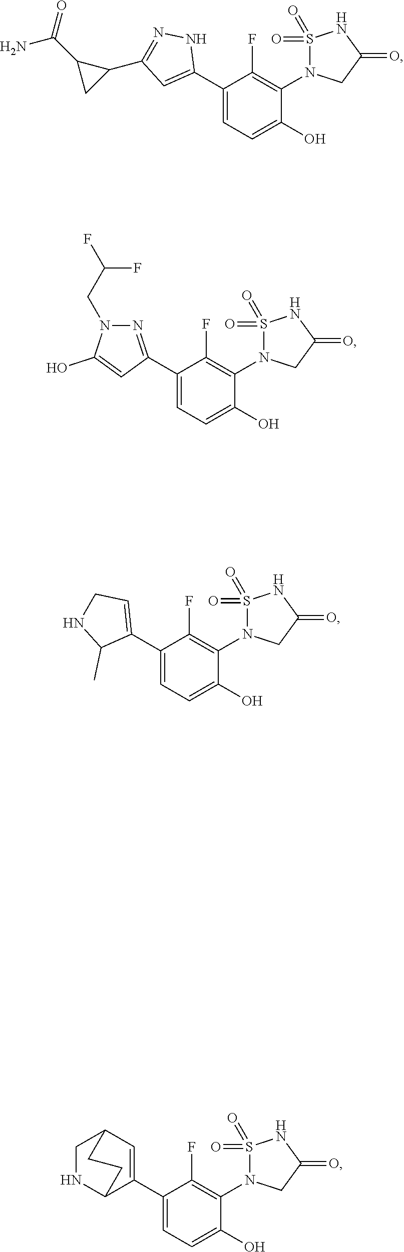 [SMILES: NC(=O)C1CC1c1cc(-c2ccc(O)c(N3CC(=O)NS3(=O)=O)c2F)[nH]n1 ; O=C1CN(c2c(O)ccc(-c3cc(O)n(CC(F)F)n3)c2F)S(=O)(=O)N1 ; CC1NCC=C1c1ccc(O)c(N2CC(=O)NS2(=O)=O)c1F ; O=C1CN(c2c(O)ccc(C3=CC4CCC3NC4)c2F)S(=O)(=O)N1]

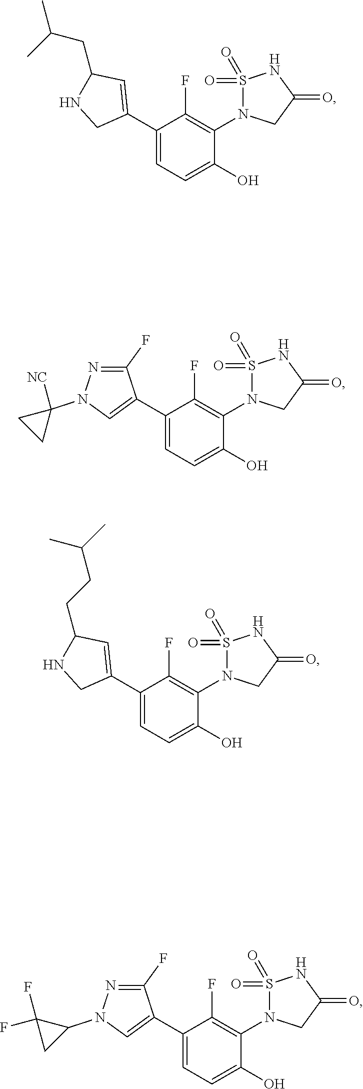 [SMILES: CC(C)CC1C=C(c2ccc(O)c(N3CC(=O)NS3(=O)=O)c2F)CN1 ; N#CC1(n2cc(-c3ccc(O)c(N4CC(=O)NS4(=O)=O)c3F)c(F)n2)CC1 ; CC(C)CCC1C=C(c2ccc(O)c(N3CC(=O)NS3(=O)=O)c2F)CN1 ; O=C1CN(c2c(O)ccc(-c3cn(C4CC4(F)F)nc3F)c2F)S(=O)(=O)N1]

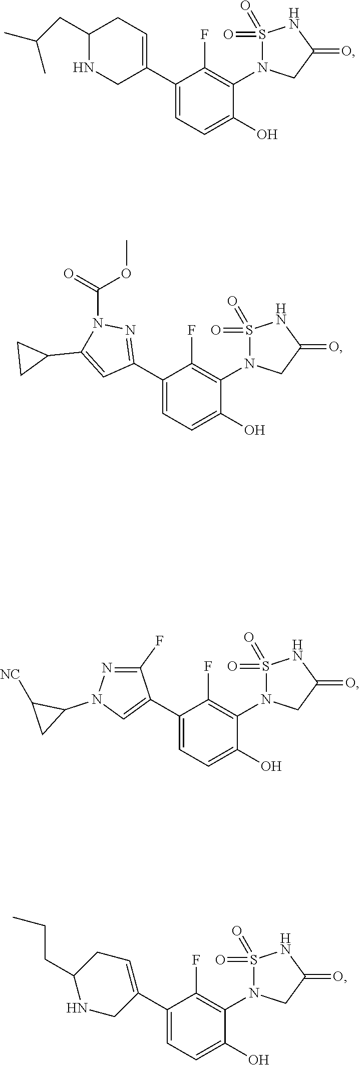 [SMILES: CC(C)CC1CC=C(c2ccc(O)c(N3CC(=O)NS3(=O)=O)c2F)CN1 ; COC(=O)n1nc(-c2ccc(O)c(N3CC(=O)NS3(=O)=O)c2F)cc1C1CC1 ; N#CC1CC1n1cc(-c2ccc(O)c(N3CC(=O)NS3(=O)=O)c2F)c(F)n1 ; CCCC1CC=C(c2ccc(O)c(N3CC(=O)NS3(=O)=O)c2F)CN1]

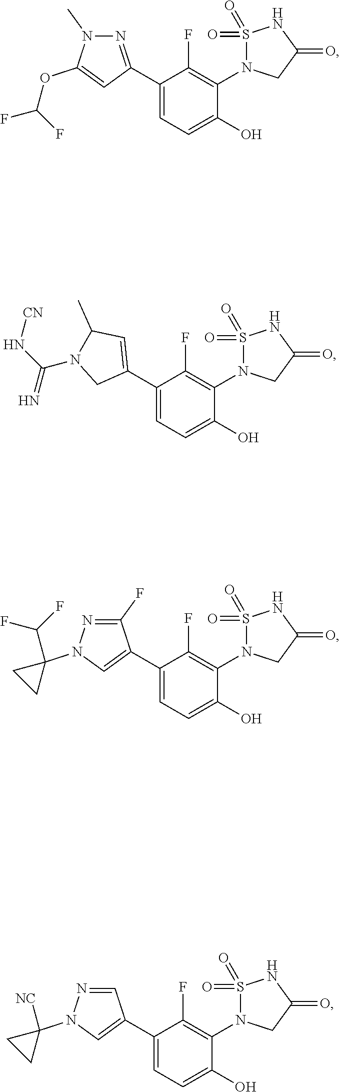 [SMILES: Cn1nc(-c2ccc(O)c(N3CC(=O)NS3(=O)=O)c2F)cc1OC(F)F ; CC1C=C(c2ccc(O)c(N3CC(=O)NS3(=O)=O)c2F)CN1C(=N)NC#N ; O=C1CN(c2c(O)ccc(-c3cn(C4(C(F)F)CC4)nc3F)c2F)S(=O)(=O)N1 ; N#CC1(n2cc(-c3ccc(O)c(N4CC(=O)NS4(=O)=O)c3F)cn2)CC1]

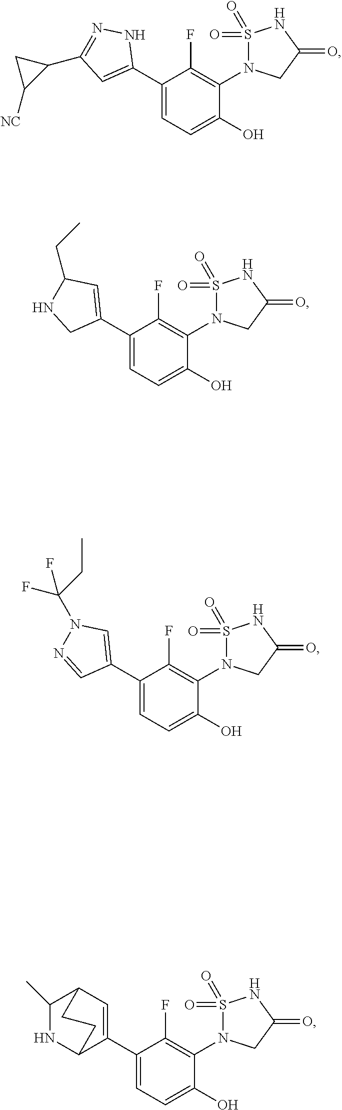 [SMILES: N#CC1CC1c1cc(-c2ccc(O)c(N3CC(=O)NS3(=O)=O)c2F)[nH]n1 ; CCC1C=C(c2ccc(O)c(N3CC(=O)NS3(=O)=O)c2F)CN1 ; CCC(F)(F)n1cc(-c2ccc(O)c(N3CC(=O)NS3(=O)=O)c2F)cn1 ; CC1NC2CCC1C=C2c1ccc(O)c(N2CC(=O)NS2(=O)=O)c1F]

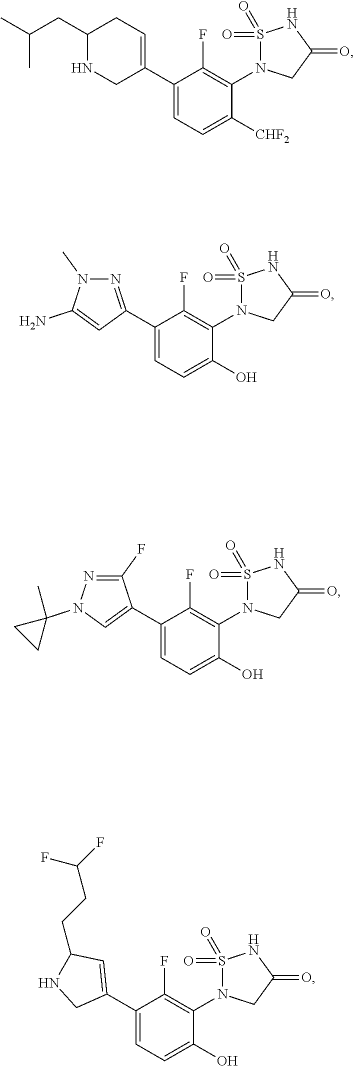 [SMILES: CC(C)CC1CC=C(c2ccc(C(F)F)c(N3CC(=O)NS3(=O)=O)c2F)CN1 ; Cn1nc(-c2ccc(O)c(N3CC(=O)NS3(=O)=O)c2F)cc1N ; CC1(n2cc(-c3ccc(O)c(N4CC(=O)NS4(=O)=O)c3F)c(F)n2)CC1 ; O=C1CN(c2c(O)ccc(C3=CC(CCC(F)F)NC3)c2F)S(=O)(=O)N1]

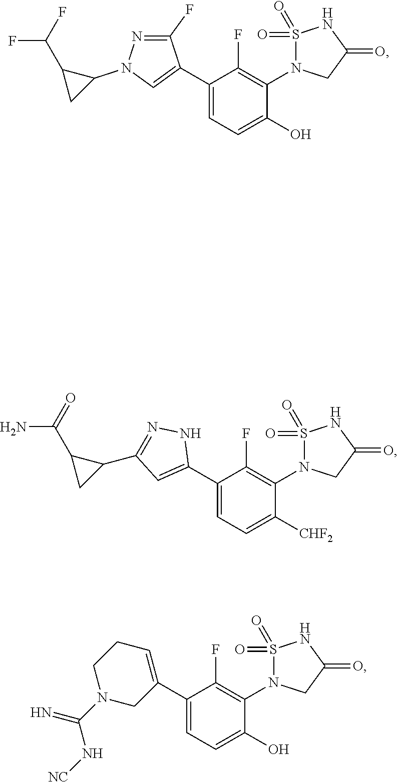 [SMILES: O=C1CN(c2c(O)ccc(-c3cn(C4CC4C(F)F)nc3F)c2F)S(=O)(=O)N1 ; NC(=O)C1CC1c1cc(-c2ccc(C(F)F)c(N3CC(=O)NS3(=O)=O)c2F)[nH]n1 ; N#CNC(=N)N1CCC=C(c2ccc(O)c(N3CC(=O)NS3(=O)=O)c2F)C1]

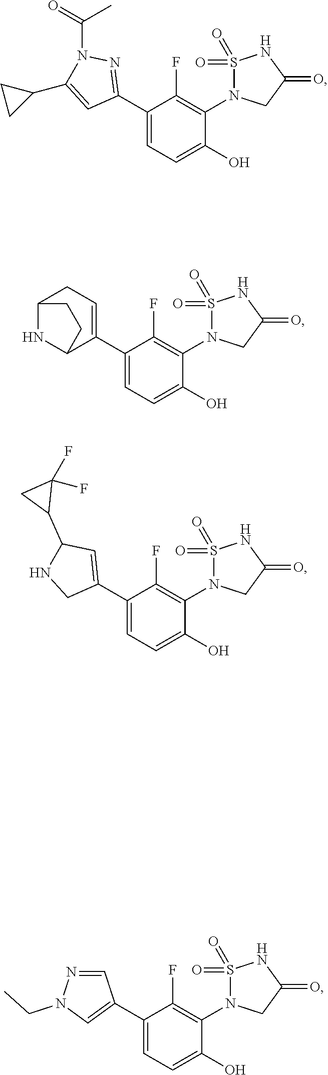 [SMILES: CC(=O)n1nc(-c2ccc(O)c(N3CC(=O)NS3(=O)=O)c2F)cc1C1CC1 ; O=C1CN(c2c(O)ccc(C3=CCC4CCC3N4)c2F)S(=O)(=O)N1 ; O=C1CN(c2c(O)ccc(C3=CC(C4CC4(F)F)NC3)c2F)S(=O)(=O)N1 ; CCn1cc(-c2ccc(O)c(N3CC(=O)NS3(=O)=O)c2F)cn1]

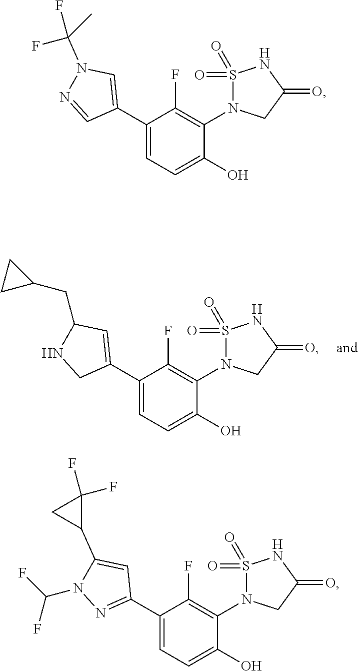 [SMILES: CC(F)(F)n1cc(-c2ccc(O)c(N3CC(=O)NS3(=O)=O)c2F)cn1 ; O=C1CN(c2c(O)ccc(C3=CC(CC4CC4)NC3)c2F)S(=O)(=O)N1 ; "and" ; O=C1CN(c2c(O)ccc(-c3cc(C4CC4(F)F)n(C(F)F)n3)c2F)S(=O)(=O)N1]

or a pharmaceutically acceptable salt or solvate thereof.

In an aspect is provided a compound having the formula A-L^{AB}-B wherein
A is a monovalent form of a compound described herein;
L^{AB} is a covalent linker bonded to A and B; and
B is a monovalent form of a degradation enhancer (i.e., degradation tag).

A “degradation enhancer” or “degradation tag” is a compound capable of binding a ubiquitin ligase protein (e.g., E3 ubiquitin ligase protein) or a compound capable of binding a protein that is capable of binding to a ubiquitin ligase protein to form a protein complex capable of conjugating a ubiquitin protein to a target protein. In embodiments, the degradation enhancer is capable of binding to an E3 ubiquitin ligase protein or a protein complex comprising an E3 ubiquitin ligase protein. In embodiments, the degradation enhancer is capable of binding to an E2 ubiquitin-conjugating enzyme. In embodiments, the degradation enhancer is capable of binding to a protein complex comprising an E2 ubiquitin-conjugating enzyme and an E3 ubiquitin ligase protein.

In embodiments, the degradation enhancer is capable of binding a protein selected from E3A, mdm2, APC, EDD1, SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HER5, HERC6, HUWE1, ITCH, NEDD4, NEDD4L, PPIL2, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE3D, UBE4A, UBE4B, UBOX5, UBR5, VHL (von-Hippel-Lindau ubiquitin ligase), WWP1, WWP2, Parkin, MKRN1, CMA (chaperon-mediated autophage), SCFb-TRCP (Skip-Cullin-F box (Beta-TRCP) ubiquitin complex), b-TRCP (b-transducing repeat-containing protein), cIAP1 (cellular inhibitor of apoptosis protein 1), APC/C (anaphase-promoting complex/cyclosome), CRBN (cereblon), CUL4-RBX1-DDB1-CRBN (CRL4^{CRBN}) ubiquitin ligase, XIAP, IAP, KEAP1, DCAF15, RNF114, DCAF16, AhR, SOCS2, KLHL12, UBR2, SPOP, KLHL3, KLHL20, KLHDC2, SPSB1, SPSB2, SPSB4, SOCS6, FBXO4, FBXO31, BTRC, FBW7, CDC20, PML, TRIM21, TRIM24, TRIM33, GID4, avadomide, iberdomide, and CC-885.

In embodiments, the degradation enhancer is capable of binding a protein selected from UBE2A, UBE2B, UBE2C, UBE2D1, UBE2D2, UBE2D3, UBE2DR, UBE2E1, UBE2E2, UBE2E3, UBE2F, UBE2G1, UBE2G2, UBE2H, UBE2I, UBE2J¹, UBE2J², UBE2K, UBE2L3, UBE2L6, UBE2L1, UBE2L2, UBE2L4, UBE2M, UBE2N, UBE20, UBE2Q1, UBE2Q2, UBE2R¹, UBE2R², UBE2S, UBE2T, UBE2U, UBE2V1, UBE2V2, UBE2W, UBE2Z, ATG3, BIRC6, and UFC1.

In embodiments, the degradation enhancer is a compound described in Ishida and Ciulli, SLAS Discovery 2021, Vol. 25(4) 484-502, which is incorporated by reference in its entirety for any purpose, for example VH032, VH101, VH298, thalidomide, bestatin, methyl bestatin, nutlin, idasanutlin, bardoxolone, bardoxolone methyl, indisulam (E7070), E7820, chloroquinoxaline sulfonamide (CQS), nimbolide, KB02, ASTX660, lenalidomide, or pomalidomide.

In embodiments, the degradation enhancer is a compound described in US20180050021, WO2016146985, WO2018189554, WO2018119441, WO2018140809, WO2018119448, WO2018119357, WO2018118598, WO2018102067, WO201898280, WO201889736, WO201881530, WO201871606, WO201864589, WO201852949, WO2017223452, WO2017204445, WO2017197055, WO2017197046, WO2017180417, WO2017176958, WO201711371, WO2018226542, WO2018223909, WO2018189554, WO2016169989, WO2016146985, CN105085620B, CN106543185B, U.S. Ser. No. 10/040,804, U.S. Pat. No. 9,938,302, U.S. Ser. No. 10/144,745, U.S. Ser. No. 10/145,848, U.S. Pat. Nos. 9,938,264, 9,632,089, 9,821,068, 9,758,522, 9,500,653, 9,765,019, 8,507,488, 8,299,057, US20180298027, US20180215731, US20170065719, US20170037004, US20160272639, US20150291562, or US20140356322, which are incorporated by reference in their entirety for any purpose.

In embodiments L^{AB} is -L^{AB1}-L^{AB2}-L^{AB3}-L^{AB4}-L^{AB5}-;
L^{AB1}, L^{AB2}, L^{AB3}, L^{AB4}, and L^{AB5} are independently a bond, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, C₁₋₆alkylene, (—O—C₁₋₆alkyl)_{z}-, (—C₁₋₆alkyl-O)_{z}—, C₂₋₆alkenylene, C₂₋₆alkynylene, C₁₋₆haloalkylene, C₃₋₁₂cycloalkylene, C₁₋₁₁heterocycloalkylene, C₆₋₁₂arylene, or C₁₋₁₁heteroarylene, wherein C₁₋₆ alkylene, C₂₋₆alkenylene, C₂₋₆alkynylene, C₁₋₆haloalkylene, C₃₋₁₂cycloalkylene, C₁₋₁₁heterocycloalkylene, C₆₋₁₂arylene, or C₁₋₁₁ heteroarylene, are optionally substituted with one, two, or three R^{20j}; wherein each C₁₋₆alkyl of (—O—C₁₋₆alkyl)_{z}- and (—C₁₋₆ alkyl-O)₂— is optionally substituted with one, two, or three R^{20j};
z is independently an integer from 0 to 10;
each R¹² is independently selected from hydrogen, C₁₋₆alkyl, C₂₋₆alkenyl, C₂₋₆alkynyl, C₃₋₆cycloalkyl, —CH₂—C₃₋₆cycloalkyl, C₂₋₉ heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl, wherein C₁₋₆ alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three R^{20d};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20e};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{20f},
each R^{20d}, R^{20e}, R^{20f}, and R^{20j} are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, C₁₋₉ heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₆cycloalkyl, —CH₂—C₃₋₆cycloalkyl, C₂₋₉ heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₉haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl; and
each R²⁵ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl.

In embodiments, L^{AB} is —(O—C₂alkyl)_{z}- and z is an integer from 1 to 10.

In embodiments, L^{AB} is —(C₂alkyl-O—)_{z}- and z is an integer from 1 to 10.

In embodiments, L^{AB} is —(CH₂)_{zz1}L^{AB2}(CH₂O)_{zz2}—, wherein L^{AB2} is a bond, a C₂₋₅heterocycloalkylene or heteroarylene, phenylene, —(C₂-C₄)alkynylene, —SO₂— or —NH—; and zz1 and zz2 are independently an integer from 0 to 10.

In embodiments, L^{AB} is —(CH₂)_{zz1}(CH₂O)_{zz2}—, wherein zz1 and zz2 are each independently an integer from 0 to 10.

In embodiments, L^{AB} is a PEG linker (e.g., divalent linker of 1 to 10 ethylene glycol subunits).

In embodiments, B is a monovalent form of a compound selected from

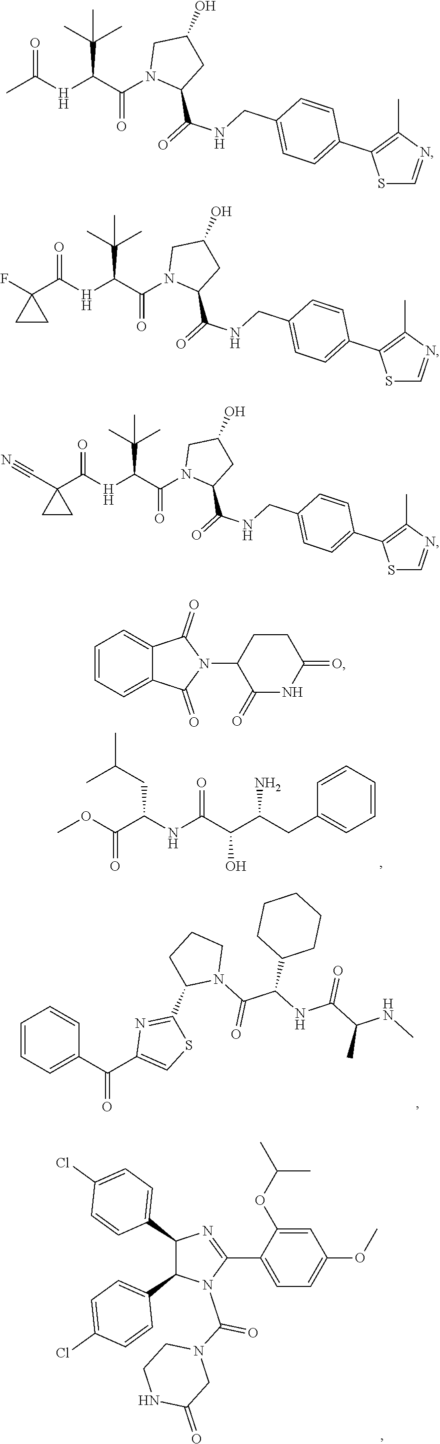

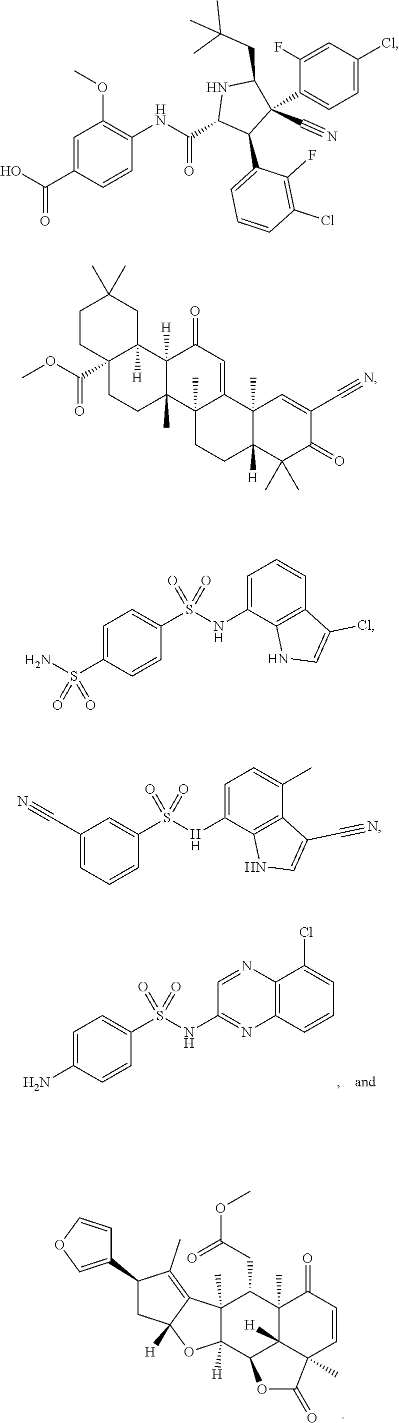

In embodiments, B is a monovalent form of a compound selected from

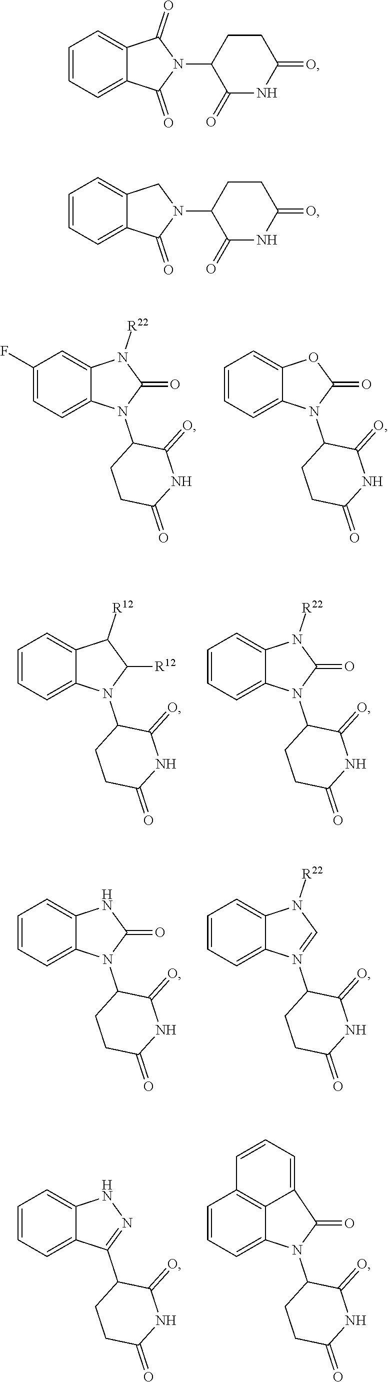

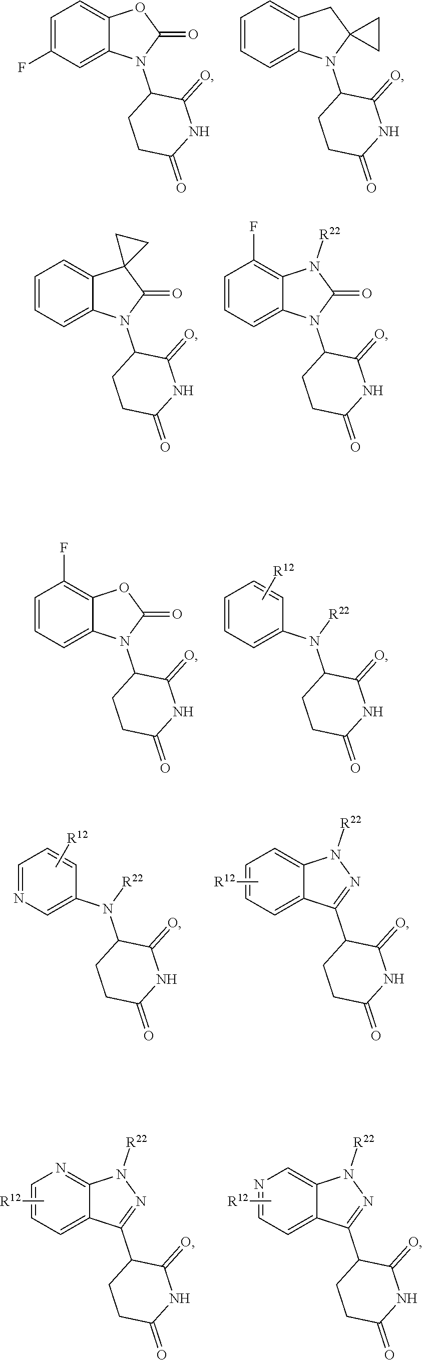

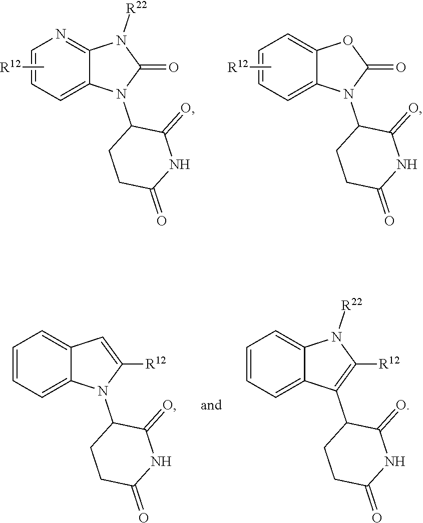

In some embodiments, the compound of formula A-L^{AB}-B is selected from

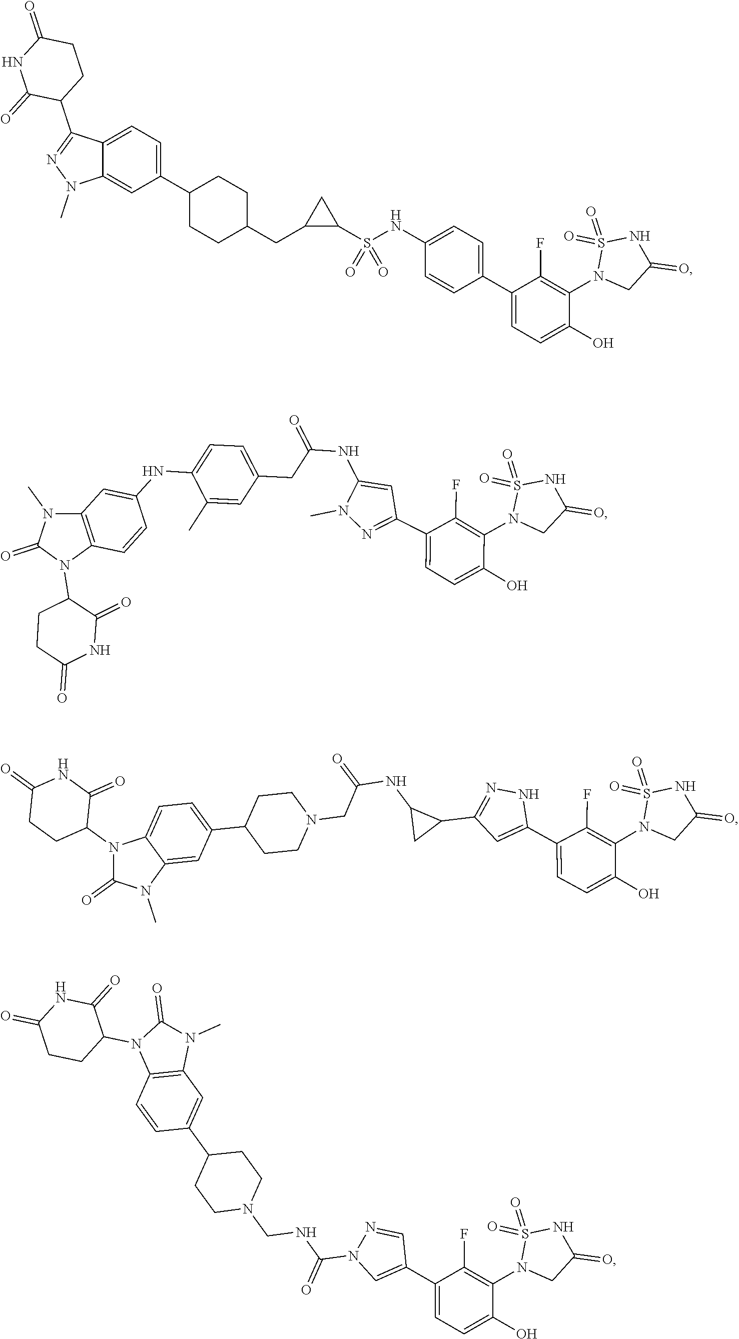 [SMILES: Cn1nc(C2CCC(=O)NC2=O)c2ccc(C3CCC(CC4CC4S(=O)(=O)Nc4ccc(-c5ccc(O)c(N6CC(=O)NS6(=O)=O)c5F)cc4)CC3)cc21 ; Cc1cc(CC(=O)Nc2cc(-c3ccc(O)c(N4CC(=O)NS4(=O)=O)c3F)nn2C)ccc1Nc1ccc2c(c1)n(C)c(=O)n2C1CCC(=O)NC1=O ; Cn1c(=O)n(C2CCC(=O)NC2=O)c2ccc(C3CCN(CC(=O)NC4CC4c4cc(-c5ccc(O)c(N6CC(=O)NS6(=O)=O)c5F)[nH]n4)CC3)cc21 ; Cn1c(=O)n(C2CCC(=O)NC2=O)c2ccc(C3CCN(CNC(=O)n4cc(-c5ccc(O)c(N6CC(=O)NS6(=O)=O)c5F)cn4)CC3)cc21]

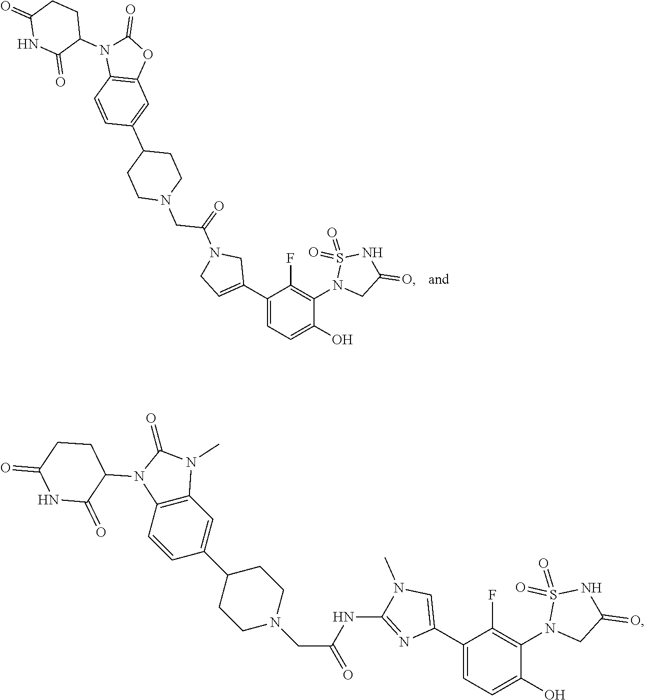 [SMILES: O=C1CCC(n2c(=O)oc3cc(C4CCN(CC(=O)N5CC=C(c6ccc(O)c(N7CC(=O)NS7(=O)=O)c6F)C5)CC4)ccc32)C(=O)N1 ; Cn1cc(-c2ccc(O)c(N3CC(=O)NS3(=O)=O)c2F)nc1NC(=O)CN1CCC(c2ccc3c(c2)n(C)c(=O)n3C2CCC(=O)NC2=O)CC1]

or a pharmaceutically acceptable salt or solvate thereof.

It shall be understood that different aspects of the invention can be appreciated individually, collectively, or in combination with each other. Various aspects of the invention described herein may be applied to any of the particular applications disclosed herein. The compositions of matter including compounds of any formulae disclosed herein in the composition section of the present disclosure may be utilized in the method section including methods of use and production disclosed herein, or vice versa.

### Further Forms of Compounds Disclosed Herein Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion, are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that does not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds described herein include isotopes of hydrogen, carbon, nitrogen, oxygen, phosphorous, sulfur, fluorine, and chloride, such as ²H, ³H, ¹³C, ¹⁴C, ¹⁵N, ¹⁷O, ¹⁸O, ³¹P, ³²P, ³⁵S, ¹⁸F, and ³⁶Cl, respectively. Compounds described herein, and pharmaceutically acceptable salts, esters, solvate, hydrates, or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate, or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds described herein, or by separately reacting a purified compound in its free form with a suitable acid or base, and isolating the salt thus formed.

### Solvates

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or MeOH. In addition, the compounds provided herein exist in unsolvated as well as solvated forms. In general, the solvated forms are considered equivalent to the unsolvated forms for the purposes of the compounds and methods provided herein.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as disclosed herein may be derived from reactions and the reactions may be modified by the use of appropriate reagents and conditions, for the introduction of the various moieties found in the formulae as provided herein. In some embodiments, the following synthetic methods may be utilized.

### General Synthetic Method 1

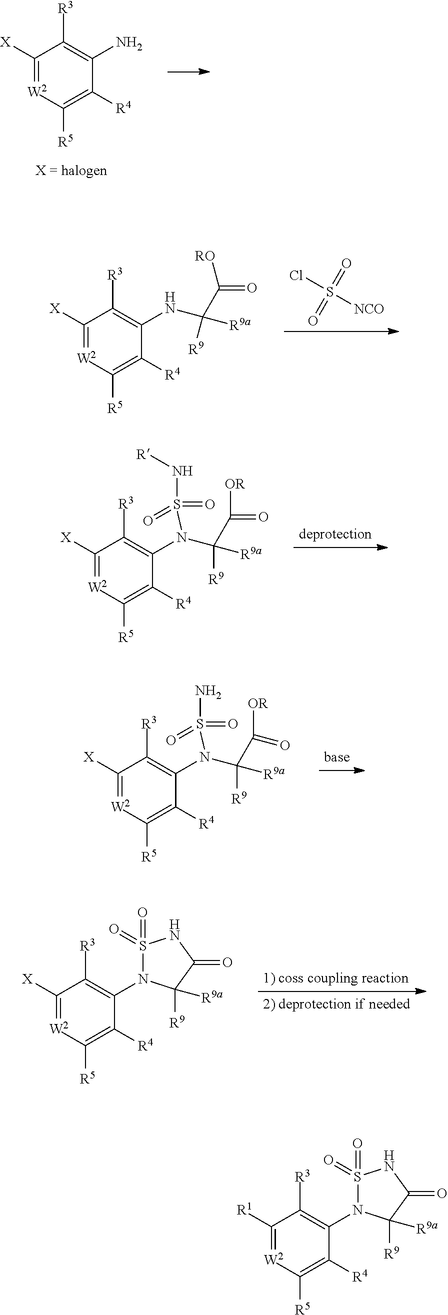

### General Synthetic Method 2

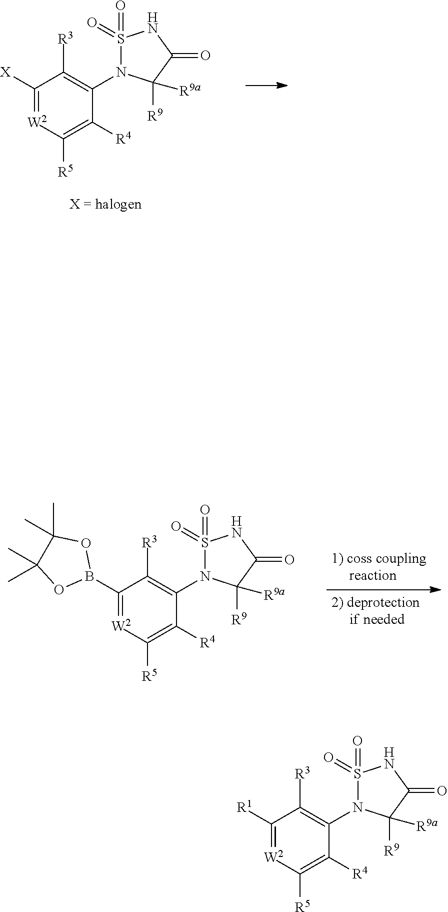

### General Synthetic Method 3

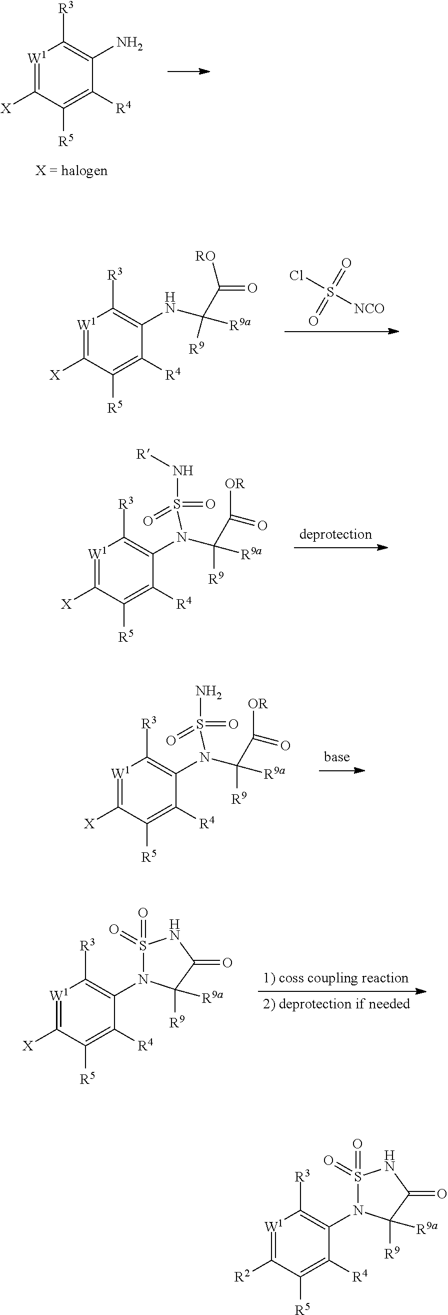

### General Synthetic Method 4

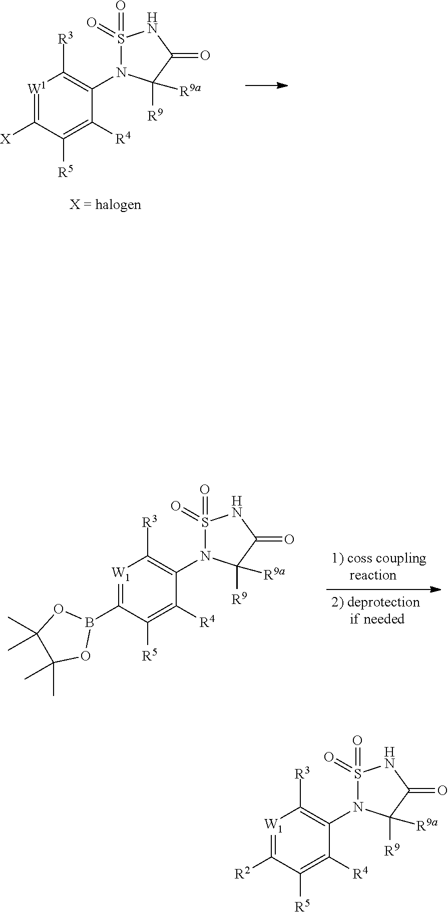

### General Synthetic Method 5

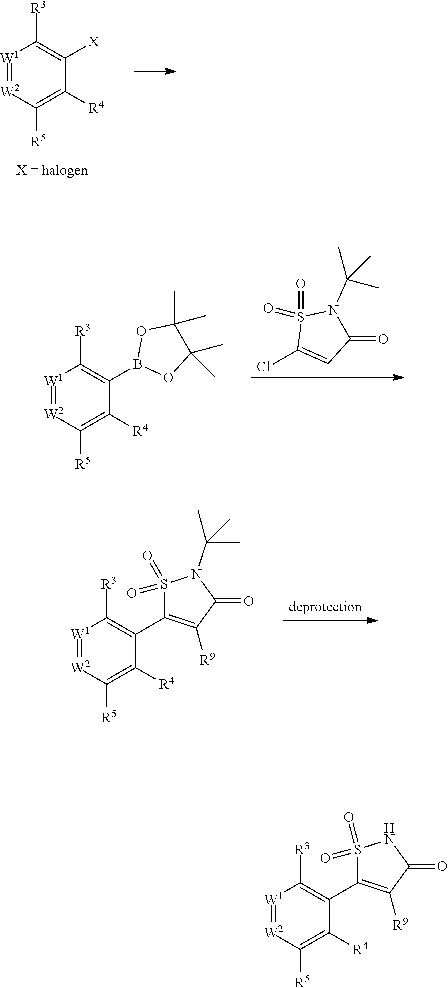

General synthetic method 6

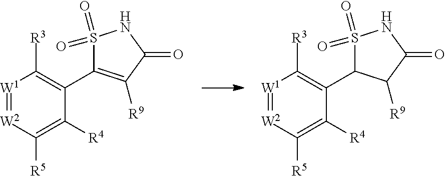

### Pharmaceutical Compositions and Methods of Administration

The compounds of compound described herein (e.g., a compound of Formula I, II, III, IV, V, VI, VII, VIII, or a sub-formulae thereof), or a pharmaceutically acceptable salt or solvate thereof, described herein are administered to subjects in a biologically compatible form suitable for administration to treat or prevent diseases, disorders or conditions. Administration of the compounds described herein can be in any pharmacological form including a therapeutically effective amount of a compound described herein (e.g., a compound of Formula I, II, III, IV, V, VI, VII, VIII, or a sub-formulae thereof), or a pharmaceutically acceptable salt or solvate thereof, alone or in combination with a pharmaceutically acceptable carrier.

In some embodiments, the compounds described herein are administered as a pure chemical. In other embodiments, the compounds described herein are combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, PA (2005)).

Accordingly, provided herein is a pharmaceutical composition comprising at least one compound described herein, or a pharmaceutically acceptable salt, together with one or more pharmaceutically acceptable excipients. The excipient(s) (or carrier(s)) is acceptable or suitable if the excipient is compatible with the other ingredients of the composition and not deleterious to the recipient (i.e., the subject) of the composition.

In some embodiments is a pharmaceutical composition comprising a pharmaceutically acceptable excipient and a compound described herein (e.g., a compound of Formula I, II, III, IV, V, VI, VII, VIII, or a sub-formulae thereof), or a pharmaceutically acceptable salt or solvate thereof.

In some embodiments of the methods described herein, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments of the methods described herein, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments of the methods described herein, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

### Methods

Compounds disclosed herein exhibiting anti-PTPN2 activity embody a variety of therapeutic utilities. In an aspect, a PTPN2 inhibitor (e.g., compound described herein) can be administered into a subject in need thereof to treat cancer. In some embodiments, a subject PTPN2 inhibitor is systemically and/or transiently (including intermittently) administered to the subject in need thereof to treat one or more types of cancer, including solid tumor and liquid tumor. In another aspect, a subject PTPN2 inhibitor is used to potentiate immunity comprising anti-tumor, anti-cancer activity, anti-viral infection activity, and/or anti-bacterial infection activity in a cell or a subject. In practicing any of the subject methods, a PTPN2 inhibitor disclosed herein can be administered as a single agent. In some embodiments, a PTPN2 inhibitor (e.g., compound described herein) is administered in combination with another agent as a single or unit dose, or as a separate dose. In some embodiments, the another agent can be a cell, including but not limited to a lymphoid cell (e.g., expressing a CAR and/or TCR). In some embodiment, the another agent can be a second agent including without limitation, chemotherapeutic agent, a radioactive agent, a small molecule agent targeting a tumor marker, an antigen-binding agent specifically binding to a tumor marker, an immune modulator, or any other second agent disclosed herein.

Not wishing to be bound by any particular theory, a subject PTPN2 inhibitor (e.g., compound described herein) may be effective in one or more of: stimulating and/or prolonging anti-tumor immunity (e.g., destabilizing Tregs, augmenting CD4+ and CD8+ T cell function, increasing the number of central memory T cells or half-life of such cells), inhibiting proliferation of cancer cells, inhibiting invasion or metastasis of cancer cells, killing cancer cells, increasing the sensitivity of cancer cells to treatment with a second antitumor agent, and reducing severity or incidence of symptoms associated with the presence of cancer cells. In some embodiments, said method comprises administering to the cancer cells a therapeutically effective amount of a PTPN2 inhibitor (e.g., compound described herein) in vivo. In some embodiments, the administration first takes place ex vivo to a population of effector cells, followed by infusing the PTPN2- inhibitor (e.g., compound described herein) treated effector cells into the subject as further detailed below.

In some embodiments, the small molecule PTPN2 inhibitor may not effect editing of (i) a gene encoding PTPN2 or (ii) an additional gene operatively linked to PTPN2 (e.g., transcription factor, intron sequence, start codon, etc.). As such, the gene and/or the additional gene may remain the same upon treatment of a cell with a small molecule PTPN2 inhibitor (e.g., compound described herein). In some embodiments, the small molecule PTPN2 inhibitor (e.g., compound described herein) may be configured to bind at least a portion of PTPN2. The small molecule may exhibit binding specificity to PTPN2 in comparison to one or more other protein tyrosine phosphatases selected from the group consisting of: PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRV, PTPRZ, PTPN1, PTPN2, PTPN3, PTPN4, PTPN5, PTPN6, PTPN7, PTPN9, PTPN11, PTPN12, PTPN13, PTPN14, PTPN18, PTPN20, PTPN21, PTPN23, DUSP1, DUSP2, DUSP4, DUSP5, DUSP6, DUSP7, DUSP8, DUSP9, DUSP10, DUSP16, MK-STYX, DUSP3, DUSP11, DUSP12, DUSP13Aa, DUSP13Ba, DUSP14, DUSP15, DUSP18, DUSP19, DUSP21, DUSP22, DUSP23, DUSP24, DUSP25, DUSP26, DUSP27b, EPM2A, RNGTT, STYX, SSH1, SSH2, SSH3, PTP4A1, PTP4A2, PTP4A3, CDCl4A, CDCl4B, CDKN3, PTP9Q22, PTEN, TPIP, TPTE, TNS, TENC1, MTM1, MTMR1, MTMR2, MTMR3, MTMR4, MTMR5, MTMR6, MTMR7, MTMR8, MTMR9, MTMR10, MTMR11, MTMR12, MTMR13, MTMR14, MTMR15, ACP1, CDC25A, CDC25B, CDC25C, EYA1, EYA1, EYA1, and EYA1. In some embodiments, a subject compound (i.e., compound described herein) specifically binds to PTPN2 relative to PTP1B. In some embodiments, a subject compound (i.e., compound described herein) binds to both PTPN2 and PTP1B. In some embodiments, a subject compound (i.e., compound described herein) selectively inhibits PTPN2 relative to PTP1B. In some embodiments, a subject compound (i.e., compound described herein) selectively inhibits both PTPN2 and PTP1B. In some cases, a subject compound (i.e., compound described herein) (e.g., compound described herein) may exhibit a half maximal inhibitory concentration (i.e., IC₅₀) of less than or equal to about 10 micromolar (μM), 5 μM, 1 μM, 950 nanomolar (nM), 900 nM, 850 nM, 800 nM, 750 nM, 700 nM, 650 nM, 600 nM, 550 nM, 500 nM, 450 nM, 400 nM, 350 nM, 300 nM, 250 nM, 200 nM, 150 nM, 100 nM, 50 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, or less for PTPN2. The small molecule PTPN2 inhibitor (e.g., compound described herein) may exhibit IC₅₀ for PTPN2 that is at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20- fold, 30-fold, 40-fold, 50-fold, 60-fold, 70-fold, 80-fold, 90-fold, 100-fold, or more potent (e.g., IC50 concentration is a lower number for PTPN2 than another PTP) than that of one or more other protein tyrosine phosphatases. In different embodiments, the small molecule PTPN2 inhibitor (e.g., compound described herein) may be configured to bind at least a portion of one or more substrates of PTPN2 selected from the group consisting of: INSR, EGFR, CSF1R, PDGFR, JAK1, JAK2, JAK3, Src family kinases, STAT1, STAT3, STAT6, FYN, LCK, variations thereof, and combinations thereof.

In some embodiments, a subject compound (i.e., compound described herein) may be conjugated to a degradation tag (i.e., degradation enhancer). A degradation tag may be configured to bind a degradation moiety having a capacity to degrade at least a portion of a target moiety that is bound by the degradation tag. For example, the target moiety is PTPN2 or the substrate of PTPN2. A degradation tag may be a biological or chemical compound, such as a simple or complex organic or inorganic molecule, peptide, peptido mimetic, protein (e.g., antibody), liposome, or a polynucleotide (e.g., small interfering RNA, short hairpin RNA, microRNA, antisense, aptamer, ribozyme, triple helix). Alternatively, a degradation tag may be synthetic. In some cases, any one of the methods described herein may utilize a small molecule degradation tag, and non-limiting examples of such small molecule degradation tag may include, but are not limited to, pomalidomide, thalidomide, lenalidomide, VHL-1, adamantane, 1-((4,4,5,5,5-pentafluoropentyl)sulfmyl)nonane, nutlin-3a, RG7112, RG7338, AMG 232, AA-115, bestatin, MV-1, LCL161, and/or analogs thereof. In some cases, the degradation tag can (i) bind to a degradation moiety such as a ubiquitin ligase (e.g., an E3 ligase such as a cereblon E3 ligase, a VHL E3 ligase, a MDM2 ligase, a TRIM21 ligase, a TRIM24 ligase, and/or a IAP ligase) and/or (ii) serve as a hydrophobic group that leads to protein misfolding of the target moiety, e.g., PTPN2. Misfolding of the target moiety may disrupt activity of the target moiety and/or increase the likelihood of degradation of the target moiety by, e.g., a degradation moiety. In some cases, a small molecule PTPN2 inhibitor (e.g., compound described herein) may be conjugated to the degradation tag via a linker. Examples of such linker may include, but are not limited to, acyclic or cyclic saturated or unsaturated carbon, ethylene glycol, amide, amino, ether, urea, carbamate, aromatic, heteroaromatic, heterocyclic, and/or carbonyl containing groups with different lengths. Exemplary molecules comprising such degradation tag and method of use thereof are provided in U.S. Patent Publication No. 2019/0336503, which is incorporated herein by reference in its entirety.

In some embodiments, a method of the disclosure provides an effective amount of a PTPN2 inhibitor (e.g., compound described herein). An effective dose refers to an amount sufficient to effect the intended application, including treatment of cancer, stimulating or prolonging anti-tumor immunity. Also contemplated in the subject methods is the use of a sub-therapeutic amount of a PTPN2 inhibitor (e.g., compound described herein) for treating an intended disease condition.

The amount of the PTPN2 inhibitor (e.g., compound described herein) administered may vary depending upon the intended application (in vitro, ex vivo, or in vivo), or the subject and cancer condition being treated, e.g., the weight and age of the subject, the severity of the cancer, the manner of administration and the like.

In some cases, a PTPN2 inhibitor (e.g., compound described herein) may be administered (e.g., systemically administered) to a subject at a dose of at least about 0.1 milligrams per kilogram (mg/kg), 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 25 mg/kg, 30 mg/kg, 35 mg/kg, 40 mg/kg, 45 mg/kg, 50 mg/kg, or more. In some cases, a PTPN2 inhibitor may be administered (e.g., systemically administered) to a subject at a dose of at most about 50 mg/kg, 45 mg/kg, 40 mg/kg, 35 mg/kg, 30 mg/kg, 25 mg/kg, 20 mg/kg, 19 mg/kg, 18 mg/kg, 17 mg/kg, 16 mg/kg, 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, 0.1 mg/kg, or less.

In some cases, upon administration (e.g., systemic administration), a mean plasma concentration of the PTPN2 inhibitor (e.g., compound described herein) in the subject may be at least about 0.1 microgram per milliliter (μg/ml), 0.2 μg/ml, 0.3 μg/ml, 0.4 μg/ml, 0.5 μg/ml, 0.6 μg/ml, 0.7 μg/ml, 0.8 μg/ml, 0.9 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 25 μg/ml, 30 μg/ml, 35 μg/ml, 40 μg/ml, 45 μg/ml, 50 μg/ml, or more. In some cases, upon administration (e.g., systemic administration), a mean plasma concentration of the PTPN2 inhibitor (e.g., compound described herein) in the subject may be at most about 50 μg/ml, 45 μg/ml, 40 μg/ml, 35 μg/ml, 30 μg/ml, 25 μg/ml, 20 μg/ml, 19 μg/ml, 18 μg/ml, 17 μg/ml, 16 μg/ml, 15 μg/ml, 14 μg/ml, 13 μg/ml, 12 μg/ml, 11 μg/ml, 10 μg/ml, 9 μg/ml, 8 μg/ml, 7 μg/ml, 6 μg/ml, 5 μg/ml, 4 μg/ml, 3 μg/ml, 2 μg/ml, 1 μg/ml, 0.9 μg/ml, 0.8 μg/ml, 0.7 μg/ml, 0.6 μg/ml, 0.5 μg/ml, 0.4 μg/ml, 0.3 μg/ml, 0.2 μg/ml, 0.1 μg/ml, or less.

In some embodiments, a PTPN2 inhibitor (e.g., compound described herein) may be used in combination with another known agent (a second agent) or therapy. Examples of such second agent may be selected from the group consisting of a chemotherapeutic agent, a radioactive agent, a small molecule agent targeting a tumor marker, an antigen-binding agent specifically binding to a tumor marker, and an immune modulator. An immune modulator may be selected from the group consisting of immunostimulatory agents, checkpoint immune blockade agents, and combinations thereof. In some embodiments, the second agent may be a checkpoint inhibitor. In some examples, the second agent may be an inhibitor of PD1, PD-L1, LAG3, CTLA4, CD160, BTLA, LAIR1, TIM3, 2B4, CD93, OX40, Siglec-15, and TIGIT. A PTPN2 inhibitor can be administered as part of a therapeutic regimen that comprises administering one or more second agents (e.g. 1, 2, 3, 4, 5, or more second agents), either simultaneously or sequentially with the PTPN2 inhibitor (e.g., compound described herein). When administered sequentially, the PTPN2 inhibitor (e.g., compound described herein) may be administered before, concurrent with, or after the one or more second agents. When administered simultaneously, the PTPN2 inhibitor (e.g., compound described herein) and the one or more second agents may be administered by the same route (e.g. injections to the same location; tablets taken orally at the same time), by a different route (e.g. a tablet taken orally while receiving an intravenous infusion), or as part of the same combination (e.g. a solution comprising the PTPN2 inhibitor (e.g., compound described herein) and one or more second agents). In some examples, a PTPN2 inhibitor (e.g., compound described herein) can be used in combination with a cell therapy, including a TFP- or CAR-expressing cell (e.g., a TFP- or CAR-expressing stem cell or lymphoid cell) described herein. In other examples, a PTPN2 inhibitor (e.g., compound described herein) can be used in combination with a non-cell based therapy, such as surgery, chemotherapy, targeted therapy (e.g., using large or small drug molecules targeting a tumor antigen other than PTPN2), radiation, and the like.

In some embodiments, a PTPN2 inhibitor (e.g., compound described herein) described herein is administered to a subject in combination with an indoleamine 2,3-dioxygenase (IDO) inhibitor. IDO is an enzyme that catalyzes the degradation of the amino acid, L-tryptophan, to kynurenine. Many cancers overexpress IDO, e.g., prostatic, colorectal, pancreatic, cervical, gastric, ovarian, head, and lung cancer. pDCs, macrophages, and dendritic cells (DCs) can express IDO. Without being bound by any particular theory, it has been reported that a decrease in L-tryptophan (e.g., catalyzed by IDO) results in an immunosuppressive milieu by inducing T-cell anergy and apoptosis. It is thought that IDO inhibitor can enhance the efficacy of a CAR-expressing cell by decreasing the suppression or death of a CAR-expressing immune cell. While the clinical trial involving the combination of pembrolizumab (an anti-PD1 antibody) and epacadostat (an IDO inhibitor) did not reach the desired end point, a PTPN2 inhibitor is expected to potentiate the therapeutic effect of IDO inhibitor. Without being bound by a particular theory, PTPN2 inhibitors (e.g., compounds described herein) destabilize the function of the already activated regulatory T cells while the IDO inhibitors prevent the activation of new regulatory T cells. Exemplary inhibitors of IDO that can be used in combination include but are not limited to 1-methyl-tryptophan, indoximod (NewLink Genetics) (see, e.g., Clinical Trial Identifier Nos. NCT01191216; NCT01792050), and INCB024360 (Incyte Corp.) (see, e.g., Clinical Trial Identifier Nos. NCT01604889; NCT01685255).

Additional agents that can be used in combination with a PTPN2 inhibitor (e.g., compound described herein) include the various categories and examples of agents listed in Table 1 below.

TABLE 1

| | |
|---|---|
| Alkylating | Examples include, but are not limited to, altretamine |
| agents | (Hexalen ® or hexamethylmelamine or HMM), |
| | bendamustine, busulfan (Busulfex ® or Myleran ®), |
| | carmustine (BiCNU ®or BCNU ®), chlorambucil, |
| | cyclophophamide (Cytoxan ® or Neosar ®), |
| | dacarbazine (DTIC-Dome ®), fotemustine, |
| | ifosfamide (Ifex ®), improsulfan, lomustine |
| | (CCNU ® or CeeNU ®), mechlorethamine or |
| | mustine (Mustardgen ®), melphalan (Alkeran ®), |
| | nimustine, piposulfan, ranimustine, semustine, |
| | streptozocin (Zanosar ®), temozolomide (Temodar ®), |
| | thiotepa (Thioplex ®), trofosfamide (Ixoten ®), and |
| | uramustine or uracil mustard |
| Anthracyclines | Examples include, but are not limited to, aclarubicin, |
| | amrubicin, daunorubicin (Daunomycin ®), daunorubicin |
| | (liposomal), doxorubicin (Adriamycin ®), doxorubicin |
| | (liposomal), epirubicin, esorubicin, idarubicin, |
| | mitoxantrone, pirarubicin, and valrubicin |
| Anti-angiogenic | Examples include, but are not limited to, aflibercept, |
| | axitinib (Inlyta ®), bevacizumab (Avastin ®), |
| | cabozantinib (Cometriq ®), everolimus (Afinitor ® or |
| | Zortress ®), lenalidomide (Revlimid ®), pazopanib |
| | (Votrient ®), ponatinib, ramucirumab (Cyramza ®), |
| | ranibizumab, regorafenib (Stivarga ®), sorafenib |
| | (Nexavar ®), sunitinib (Sutent ®), thalidomide |
| | (Synovir ® or Thalomide ®), lapatinib, and |
| | vandetanib (Caprelsa ®) |
| Antifolates | Examples include, but are not limited to, aminopterin, |
| | edatrexate, folic acid, GW1843, lometrexol, LY309887, |
| | methotrexate, nolatrexed, OSI-7904(L), pemetrexed |
| | (Alimta ®), pralatrexate, raltitrexed, trimetrexate, |
| | and ZD9331 |
| BCL-2 | Examples include, but are not limited to, ABT-199, |
| inhibitors | ABT-263, ABT-737, BH3 mimetics, gossypol, obatoclax |
| | (GX15-070), oblimersen (Genasense ®; G3139; |
| | Augmerosen ®), and venetoclax |
| Bcr-Ab1 | Examples include, but are not limited to, bafetinib |
| inhibitors | (INNO-406), bosutinib (SKI-606), dasatinib |
| | (BMS-354825), imatinib (STI-571), nilotinib (AMN-107), |
| | and ponatinib (AP-24534 or Iclusig ®) |
| Biologics | Examples include, but are not limited to, BCG |
| | (TheraCys ®, cytokines (such as INF-alfa, Aldesleukin, |
| | erythropoietin, GM-CSF, G-CSF), gene therapy (such as CT109 |
| | or Kymriah ®), MAbs (such as rituximab, alemtuzumab, |
| | ipilimumab, bevacizumab, obtinutuzumab, brentuximab), |
| | oncolytic viruses (such as T-VEC or Imlygic ®, H101), |
| | and treatment vaccines (such as sipuleucel-T or |
| | Provenge ®) |
| CDK inhibitors | Examples include, but are not limited to, A-674563, |
| | abemaciclib (LY2835219), AT-7519, AZD5438, |
| | BMS-265246, BS-181, CYC202 (roscovitine; seliciclib), |
| | dinaciclib (SCH-727965), flavopiridol (alvocidib), |
| | indirubin, JNJ-7706621, K03861, kenpaullone, |
| | LDC000067, LDC4297 (LDC044297), LY2857785, |
| | MK-8776 (SCH900776), ML167, MSC2530818, |
| | NU6027, olomoucine, ON123300, P276-00, palbociclib |
| | (PD-0332991), PHA-767491, PHA-793887, PHA-848125 |
| | (milciclib), purvalanol A, purvalanol B, R547, ribociclib |
| | (LEE011), RO-3306, senexin A, SNS-032 (BMS-387032), |
| | SU9516, TG003, THZ1, UCN-01 (7-hydroxystaurosporine; |
| | KRX-0601), wogonin, and XL413 |
| COX-2 inhibitors | Examples include, but are not limited to, celecoxib |
| | (Celebrex ®), etoricoxib, lumiracoxib (Prexige ®), |
| | parecoxib (Dynastat ®), rofecoxib, and valdecoxib |
| | (Bextra ®) |
| CTLA-4 | Examples include, but are not limited to, ipilimumab |
| inhibitors | (MDX-010) and tremelimumab (10D1) |
| DNA | Examples include, but are not limited to, |
| methyltransferase | 1-hydrazinophthalazine, 5,6-dihydro-5-azacytidine, |
| inhibitors | 5-aza-2′-deoxycytidine, 5-aza-C (5-azacytidine; azacytidine), |
| | 5-fluoro-2′-deoxycytidine, |
| | arabinosyl-5-azacytidine, decitabine, disulfiram, |
| | doxorubicin, EGCG, EGX30P, hydralazine, MG98, |
| | nanaomycin A, nanaomycin C, procainamide, procaine, |
| | psammaplin A, RG108, SGI-1027, sinefungin, |
| | thioguanine, and zebularine |
| ERK inhibitors | Examples include, but are not limited to, AG1478, AG99, |
| | andrographolide, apigenin, BAY 43-9006, CAY10561, |
| | DEL-22379, ERK inhibitor III, ERK5-IN-1, FR 180204, |
| | GDC0994, GDC-0994, GW5074, hypericin, ISIS 5132, |
| | KO947, MK-8353 (SCH900353), ML-9, PD169316, |
| | PD173074, pluripotin, purvalanol, pyrazolylpyrrole ERK |
| | inhibitor, SB203580, SC-1, SCH772984, SL327, |
| | SP600125, SU4984, ulixertinib (BVD-523, VRT752271), |
| | VX-11e (ERK-11e; TCS ERK 11e), and XMD 8-92 |
| Farnesyltransferase | Examples include, but are not limited to, BMS-214662, |
| inhibitors | CP-609754, DK8G557, FTI-227, L744832, L778123, |
| | lonafarnib (Sarasar ®; SCH66336), manumycin A, and |
| | R115777 (Zarnestra ®; tipifarnib |
| FLT3 inhibitors | Examples include, but are not limited to, crenolanib |
| | besylate (CP-868596-26), gilteritinib (ASP2215), |
| | lestaurtinib (CEP-701), midostaurin (Rydapt ®, PKC412), |
| | pexidartinib (PLX3397), ponatinib (AP24534), quizartinib |
| | (AC220), SKLB1028, sorafenib (Nexavar ®), sunitinib |
| | (Sutent ®, SU11248), and XL999 |
| HDAC inhibitors | Examples include, but are not limited to, 4SC-202, |
| | Abexinostat (PCI-24781), apicidin, AR-42, ATRA, |
| | Belinostat (PXD101), BG-45, BRD73954, CAY10603, |
| | CG200745, Chidamide, CHR-2845, CHR-3996, |
| | Citarinostat (ACY-241), CUDC-101, CUDC-907, curcumin, |
| | dacinostat (LAQ824), droxinostat, Entinostat (MS-275), |
| | FR901228, Givinostat (ITF2357), HBI-8000, HPOB, |
| | ITSA-1, Kevetrin, LAQ824, largazole, LMK-235, |
| | M344, MC-1568, m-carboxyxinnamic acid |
| | bishydroxamide, ME-344, Mocetinostat (MGCD0103), |
| | nexturastat A, oxamflatin, Panobinostat (LBH589), |
| | pracinostat (SB939), pyroxamide, Quisinostat |
| | (JNJ-26481585), Resminostat (4SC-201), RG2833 |
| | (RGFP109), RGFP966, ricolinostat (ACY-1215), |
| | Romidepsin (FK228; Depsipeptide), Romidepsin (Istodax), |
| | santacruzamate A (CAY10683), SB939, SBHA, scriptaid, |
| | sulforaphane, tacedinaline (CI994) PCI-34051, tasquinimod, |
| | TMP-195, TMP-269, trapoxin A, trichostatin A, tubacin, |
| | tubastatin A, Valproic acid (as Mg valproate), and |
| | Vorinostat (SAHA |
| Hedgehog | Examples include, but are not limited to, AY 9944, |
| signaling | BMS-833923 (XL139), GANT58, GANT61, HPI-4, JK184, |
| inhibitors | LEQ 506, PF-04449913, SANT-1, SANT-2, sonidegib |
| | (LDE-225; Odomzo ®), TAK-441, and vismodegib |
| | (IPI-926; Erivedge ®) |
| HIF inhibitors | Examples include, but are not limited to, |
| | 2-methoxyestradiol, bortezomib, camptothecin, |
| | echinomycin, ENMD-1198, miltefosine, perifosine, |
| | romidepsin, and temsirolimus |
| Hormone therapies | Examples include, but are not limited to, 4(5)-imidazoles, |
| | 4-hydroxytamoxifen, aminoglutethimide, anastrozole |
| | (Arimidex ®), bicalutamide megestrol acetate, |
| | diethylstilbestrol, estrace, exemestane (Aromasin ®), |
| | fluoxymesterone, flutamide, fluvestrant (Faslodex ®), |
| | goserelin (Zoladex ®), keoxifene, letrozole (Femara ®), |
| | leuprorelin, LY 117018, medroxyprogesterone acetate, |
| | nilutamide, octreotide, onapristone, polyestradiol |
| | phosphate, raloxifene, tamoxifen, toremifene, toremifene |
| | (Fareston ®), and trioxifene |
| MEK inhibitors | Examples include, but are not limited to, AR-119/ |
| | RDEA119 (BAY 869766), arctigenin, ARRY-438162, |
| | AS-701173, AS-701255, AS703026, AZD6244 |
| | (ARRY-142886), AZD8330 (ARRY-704), binimetinib |
| | (MEK162), CI-1040 (PD184352), E6201, GDC-0623, |
| | GDC-0973 (XL518; cobimetinib), hypothemycin, |
| | PD0325901, PD0325901, PD181461, PD98059, primasertib, |
| | refametinib, RO09-2210, RO4927350, RO4987655, |
| | RO5068760, RO5126766, selumetinib, TAK-733, |
| | trametinib (GSK1120212), U0126, and WX-554 |
| Mitotic inhibitors | Examples include, but are not limited to, colchicine, |
| | glaziovianin A, griseofulvin, podophyllotoxin, taxanes, |
| | vinblastine, vincristine, vindesine, and vinorelbine |
| mTOR inhibitors- | Examples include, but are not limited to, ABT578, |
| immune enhancing | AXD2014, AZD8055, BEZ235, CC-223, everolimus |
| amount which is | (RAD001), GSK2126458, INK128 (MLN-0128), |
| typically below | Ku-0063794, LY294002, NVP-BEZ235, OSI-027, |
| the therapeutic | PI-103, PP242, rapamycin (Sirolimus ®), ridaforolimus |
| dose | (Deforolimus ®; AP-23573), temsirolimus (CCI-779), |
| | and XL765 |
| Multi-kinase | Examples include, but are not limited to, AT9283, |
| inhibitors | dasatinib, lestaurtinib, midostaurin (Rydapt ®), motesanib, |
| | neratinib (HKI-272), nilotinib, pazopanib, regorafenib, |
| | sorafenib, sunitinib, vandetanib, and XL184 |
| Nitrogen mustards | Examples include, but are not limited to, bendamustine, |
| | chlorambucil, chlornaphazine, cyclophosphamide, |
| | estramustine, ifosfamide, mechlorethamine, mechlorethamine |
| | oxide hydrochloride, melphalan, mustine, novembichin, |
| | phenesterine, prednimustine, trofosfamide, uracil mustard, |
| | and uramustine |
| DNA synthesis | Examples include, but are not limited to, 3,4-dihydroxy- |
| inhibitors | benzylamine, 5-fluorodeoxyuridine, cladribine, cytarabine, |
| | daunorubicin, etoposide, flutarabine, hydroxyurea, |
| | idarubicin, methotrexate, mitoxantrone, and pralatrexate |
| PD-1 inhibitors | Example include, but are not limited to, AMP-224, |
| | AMP-514, atezolizumab (RG7446; MPDL3280A), |
| | BMS-936559, durvalumab, MAB005, MDX-1106, |
| | MEDI4736, MSB0010718C (A09-246-2), nivolumab |
| | (BMS936558), pembrolizumab (Lambrolizumab ®; |
| | MK-3475; KEYTRUDA ®), and pidilizumab (CT-011) |
| | Anti PD-1 antibodies: Examples include, but are not limited |
| | to, ABCAM ® (AB137132), AFFYMETRIX EBIOSCIENCE |
| | (J105; J116; M1H4), Amplimmune (AMP 514), avelumab |
| | (Bavencio ®), and BIOLEGEND ® (EH12.2H7; RMPI-14) |
| RAF inhibitors | Examples include, but are not limited to, AZ 628, |
| | CCT196969, CEP-32496, dabrafenib (GSK2118436), |
| | encorafenib (LGX818), GDC-0879, GW5074, LY3009120, |
| | MLN2480, NVP-BHG712, PLX-4720, PLX-7904, RAF265, |
| | RO5126766 (CH5126766), SB590885, sorafenib tosylate, |
| | TAK-632, vemurafenib (PLX4032; RG7204), and ZM336372 |
| RAS inhibitors | Examples include, but are not limited to, deltarasin, lonafarnib, |
| | reolysin, salirasib, siG12D LODER, SML-8-73-1, and tipifarnib |
| Others | Examples include, but are not limited to, 5-aminolevulinic |
| | acid, afatinib, alectinib, altretamine, anti-CD3 and/or anti-CD33 |
| | antibodies (e.g. visilizumab, gemtuzumab, AMG330), |
| | antiproliferative antibodies, aprepitant, arsenic trioxide, |
| | benzodopa, biological response modifiers, bisphosphonates |
| | (such as etridonic, clodronic, tiludronic, pamidronic, alendronic, |
| | ibandronic, risedronic, risedronic, zoledronic acid), bleomycin, |
| | blinatumomab (AMG103; Blincyto ®), brigatinib |
| | (Alunbrig ®), buserelin, cabazitaxel, carboquone, ceritinib, |
| | crizotinib, dactinomycin, degarelix, denosumab, |
| | dexamethasone, difluoromethylornithine (DFMO), |
| | dronabinol, enasidenib (AG-221), epothilone A or B, |
| | eribulin, erlotinib, gefitinib, gonadorelin agonists, |
| | granisetron, heparanase inhibitors, histone deacetylase |
| | inhibitors, histrelin, ibrutinib, inotuzumab ozogamicin |
| | (Besponsa ®), ixabepilone, lenvatinib, leuprolide, |
| | levoleucovorin, matrix metalloproteinase inhibitors (such |
| | as marimastat (BB-2516), prinomastat (AG3340), |
| | BMS-279251, BAY 12-9566, TAA211, MMI270B, AAJ996), |
| | methionine aminopeptidase inhibitors, meturedopa, |
| | mitomycin, mitotane, nabilone, necitumumab, niraparib |
| | (Zejula ®), olaparib (AZD-2281; Lynparza ®), osimertinib |
| | (Tagrisso ®), palonosetron, panitumumab, pertuzumab, |
| | porfimer sodium, revlimid, thalidomide, tipiracil, |
| | trabectedin, trastuzumab, triethylenemelamine, |
| | triethylenephosphoramide, triethylenethiophosphoramide, |
| | trimethylolomelamine, and uredopa |

In embodiments, a compound described herein (e.g., PTPN2 inhibitor) may be administered alone or in combination or in conjunction with another therapy or another agent. By “combination” it is meant to include (a) formulating a subject composition containing a subject compound (i.e., compound described herein) together with another agent, and (b) using the subject composition separate from the another agent as an overall treatment regimen. By “conjunction” it is meant that the another therapy or agent is administered either simultaneously, concurrently or sequentially with a subject composition comprising a compound disclosed herein, with no specific time limits, wherein such conjunctive administration provides a therapeutic effect.

In some embodiment, a subject treatment method (e.g., method including a compound described herein) is combined with surgery, cellular therapy, chemotherapy, radiation, and/or immunosuppressive agents. Additionally, compositions of the present disclosure (e.g., compound described herein) can be combined with other therapeutic agents, such as other anti-cancer agents, anti-allergic agents, anti-nausea agents (or anti-emetics), pain relievers, cytoprotective agents, immunostimulants, immunomodulatory agents, and combinations thereof.

In one embodiment, a subject treatment method (e.g., method including a compound described herein) is combined with a chemotherapeutic agent.

Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). Additional chemotherapeutic agents contemplated for use in combination include busulfan (Myleran®), busulfan injection (Busulfex®), cladribine (Leustatin®), cyclophosphamide (Cytoxan® or Neosar®), cytarabine, cytosine arabinoside (Cytosar-U®), cytarabine liposome injection (DepoCyt®), daunorubicin hydrochloride (Cerubidine®), daunorubicin citrate liposome injection (DaunoXome®), dexamethasone, doxorubicin hydrochloride (Adriamycin®, Rubex®), etoposide (Vepesid®), fludarabine phosphate (Fludara®), hydroxyurea (Hydrea®), Idarubicin (Idamycin®), mitoxantrone (Novantrone®), Gemtuzumab Ozogamicin (Mylotarg®), anastrozole (Arimidex®), bicalutamide (Casodex®), bleomycin sulfate (Blenoxane®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), dacarbazine (DTIC-Dome®), dactinomycin (Actinomycin D, Cosmegan), dexamethasone, docetaxel (Taxotere®), 5-fluorouracil (Adrucil®, Efudex®), flutamide (Eulexin®), tezacitibine, Gemcitabine (difluorodeoxycitidine), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/M4X-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with a compound of the present invention include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary antimetabolites include, without limitation, pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), azacitidine (Vidaza®), decitabine and gemcitabine (Gemzar®). Preferred antimetabolites include, cytarabine, clofarabine and fludarabine.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, RevimmuneT), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylnelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

In an aspect, compositions provided herein can be administered in combination with radiotherapy such as radiation. Whole body radiation may be administered at 12 Gy. A radiation dose may comprise a cumulative dose of 12 Gy to the whole body, including healthy tissues. A radiation dose may comprise from 5 Gy to 20 Gy. A radiation dose may be 5 Gy, 6 Gy, 7 Gy, 8 Gy, 9 Gy, 10 Gy, 11 Gy, 12, Gy, 13 Gy, 14 Gy, 15 Gy, 16 Gy, 17 Gy, 18 Gy, 19 Gy, or up to 20 Gy. Radiation may be whole body radiation or partial body radiation. In the case that radiation is whole body radiation it may be uniform or not uniform. For example, when radiation may not be uniform, narrower regions of a body such as the neck may receive a higher dose than broader regions such as the hips.

Where desirable, an immunosuppressive agent can be used in conjunction with a subject treatment method. Exemplary immunosuppressive agents include but are not limited to cyclosporin, azathioprine, methotrexate, mycophenolate, and FK506, antibodies, or other immunoablative agents such as CAMPATH, anti-CD3 antibodies (e.g., muromonab, otelixizumab) or other antibody therapies, cytoxin, fludarabine, cyclosporin, FK506, rapamycin, mycophenolic acid, steroids, FR901228, cytokines, and irradiation, peptide vaccine, and any combination thereof. In accordance with the presently disclosed subject matter, the above-described various methods can comprise administering at least one immunomodulatory agent. In certain embodiments, the at least one immunomodulatory agent is selected from the group consisting of immunostimulatory agents, checkpoint immune blockade agents (e.g., blockade agents or inhibitors of immune checkpoint genes, such as, for example, PD-1, PD-L1, CTL A-4, IDO, TIM3, LAG3, TIGIT, BTLA, VISTA, ICOS, KIRs and CD39), radiation therapy agents, chemotherapy agents, and combinations thereof. In some embodiments, the immunostimulatory agents are selected from the group consisting of IL-12, an agonist costimulatory monoclonal antibody, and combinations thereof. In one embodiment, the immunostimulatory agent is IL-12. In some embodiments, the agonist costimulatory monoclonal antibody is selected from the group consisting of an anti-4-11BB antibody (e.g., urelumab, PF-05082566), an anti-OX40 antibody (pogalizumab, tavolixizumab, PF-04518600), an anti-ICOS antibody (BMS986226, MEDI-570, GSK3359609, JTX-2011), and combinations thereof. In one embodiment, the agonist costimulatory monoclonal antibody is an anti-4-1 BB antibody. In some embodiments, the checkpoint immune blockade agents are selected from the group consisting of anti-PD-L1 antibodies (atezolizumab, avelumab, durvalumab, BMS-936559), anti-CTLA-4 antibodies (e.g., tremelimumab, ipilimumab), anti-PD-1 antibodies (e.g., pembrolizumab, nivolumab), anti-LAG3 antibodies (e.g., C9B7W, 410C9), anti-B7-H3 antibodies (e.g., DS-5573a), anti-TIM3 antibodies (e.g., F38-2E2), and combinations thereof. In one embodiment, the checkpoint immune blockade agent is an anti-PD-L1 antibody. In some cases, a compound of the present disclosure can be administered to a subject in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, expanded cells can be administered before or following surgery. Alternatively, compositions comprising a compound described herein can be administered with immunostimulants. Immunostimulants can be vaccines, colony stimulating agents, interferons, interleukins, viruses, antigens, co-stimulatory agents, immunogenicity agents, immunomodulators, or immunotherapeutic agents. An immunostimulant can be a cytokine such as an interleukin. One or more cytokines can be introduced with modified cells provided herein. Cytokines can be utilized to boost function of modified T lymphocytes (including adoptively transferred tumor-specific cytotoxic T lymphocytes) to expand within a tumor microenvironment. In some cases, IL-2 can be used to facilitate expansion of the modified cells described herein. Cytokines such as IL-15 can also be employed. Other relevant cytokines in the field of immunotherapy can also be utilized, such as IL-2, IL-7, IL-12, IL-15, IL-21, or any combination thereof. An interleukin can be IL-2, or aldeskeukin. Aldesleukin can be administered in low dose or high dose. A high dose aldesleukin regimen can involve administering aldesleukin intravenously every 8 hours, as tolerated, for up to about 14 doses at about 0.037 mg/kg (600,000 IU/kg). An immunostimulant (e.g., aldesleukin) can be administered within 24 hours after a cellular administration. An immunostimulant (e.g., aldesleukin) can be administered in as an infusion over about 15 minutes about every 8 hours for up to about 4 days after a cellular infusion. An immunostimulant (e.g., aldesleukin) can be administered at a dose from about 100,000 IU/kg, 200,000 IU/kg, 300,000 IU/kg, 400,000 IU/kg, 500,000 IU/kg, 600,000 IU/kg, 700,000 IU/kg, 800,000 IU/kg, 900,000 IU/kg, or up to about 1,000,000 IU/kg. In some cases, aldesleukin can be administered at a dose from about 100,000 IU/kg to 300,000 IU/kg, from 300,000 IU/kg to 500,000 IU/kg, from 500,000 IU/kg to 700,000 IU/kg, from 700,000 IU/kg to about 1,000,000 IU/kg.

In some other embodiments, any of the compounds herein that is capable of modulating a PTPN2 protein may be administered in combination or in conjunction with one or more pharmacologically active agents including but not limited to: (1)an inhibitor of MEK (e.g., MEK1, MEK2) or of mutants thereof (e.g., trametinib, cobimetinib, binimetinib, selumetinib, refametinib); (2) an inhibitor of epidermal growth factor receptor (EGFR) and/or of mutants thereof (e.g., afatinib, erlotinib, gefitinib, lapatinib, cetuximab panitumumab, osimertinib, olmutinib, EGF-816); (3) an immunotherapeutic agent (e.g., checkpoint immune blockade agents, as disclosed herein); (4) a taxane (e.g., paclitaxel, docetaxel); (5) an anti-metabolite (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); (6) an inhibitor of FGFR1 and/or FGFR2 and/or FGFR3 and/or of mutants thereof (e.g., nintedanib); (7) a mitotic kinase inhibitor (e.g., a CDK4/6 inhibitor, such as, for example, palbociclib, ribociclib, abemaciclib); (8) an anti-angiogenic drug (e.g., an anti-VEGF antibody, such as, for example, bevacizumab); (9) a topoisomerase inhibitor (e.g. epipodophyllotoxins such as for example etoposide and etopophos, teniposide, amsacrin, topotecan, irinotecan, mitoxantrone); (10) a platinum-containing compound (e.g. cisplatin, oxaliplatin, carboplatin); (11) an inhibitor of ALK and/or of mutants thereof (e.g. crizotinib, alectinib, entrectinib, brigatinib); (12) an inhibitor of c-MET and/or of mutants thereof (e.g., K252a, SU11274, PHA665752, PF2341066); (13) an inhibitor of BCR-ABL and/or of mutants thereof (e.g., imatinib, dasatinib, nilotinib); (14) an inhibitor of ErbB2 (Her2) and/or of mutants thereof (e.g., afatinib, lapatinib, trastuzumab, pertuzumab); (15) an inhibitor of AXL and/or of mutants thereof (e.g., R428, amuvatinib, XL-880); (16) an inhibitor of NTRK1 and/or of mutants thereof (e.g., Merestinib); (17) an inhibitor of RET and/or of mutants thereof (e.g., BLU-667, Lenvatinib); (18) an inhibitor of A-Raf and/or B-Raf and/or C-Raf and/or of mutants thereof (RAF-709, LY-3009120); (19) an inhibitor of ERK and/or of mutants thereof (e.g., ulixertinib); (20) an MDM2 inhibitor (e.g., HDM-201, NVP-CGM097, RG-71 12, MK-8242, RG-7388, SAR405838, AMG-232, DS-3032, RG-7775, APG-115); (21) an inhibitor of mTOR (e.g., rapamycin, temsirolimus, everolimus, ridaforolimus); (22) an inhibitor of BET (e.g., I-BET 151, I-BET 762, OTX-015, TEN-010, CPI-203, CPI-0610, olionon, RVX-208, ABBC-744, LY294002, AZD5153, MT-1, MS645); (23) an inhibitor of IGF1/2 and/or of IGF1-R (e.g., xentuzumab, MEDI-573); (24) an inhibitor of CDK9 (e.g., DRB, flavopiridol, CR8, AZD 5438, purvalanol B, AT7519, dinaciclib, SNS-032); (25) an inhibitor of farnesyl transferase (e.g., tipifarnib); (26) an inhibitor of SHIP pathway including SHIP2 inhibitor (e.g., 6-(4-amino-4-methylpiperidin-1-yl)-3-(2,3-dichlorophenyl)pyrazin-2-amine), as well as SHIP1 inhibitors; (27) an inhibitor of SRC (e.g., dasatinib); (28) an inhibitor of JAK (e.g., tofacitinib); (29) a PARP inhibitor (e.g. Olaparib, Rucaparib, Niraparib, Talazoparib), (30) a BTK inhibitor (e.g. Ibrutinib, Acalabrutinib, Zanubrutinib), (31) a ROS1 inhibitor (e.g., entrectinib), (32) an inhibitor of FLT3, HDAC, VEGFR, PDGFR, LCK, Bcr-Abl or AKT, or (33) an inhibitor of KrasGl2C mutant (e.g., including but not limited to AMG510, MRTX849, and any covalent inhibitors binding to the cysteine residue 12 of Kras, the structures of these compounds are publicly known)(e.g., an inhibitor of Ras G12C as described in US20180334454, US20190144444, US20150239900, U.S. Ser. No. 10/246,424, US20180086753, WO2018143315, WO2018206539, WO20191107519, WO2019141250, WO2019150305, U.S. Pat. No. 9,862,701, US20170197945, US20180086753, U.S. Ser. No. 10/144,724, US20190055211, US20190092767, US20180127396, US20180273523, U.S. Ser. No. 10/280,172, US20180319775, US20180273515, US20180282307, US20180282308, WO2019051291, WO2019213526, WO2019213516, WO2019217691, WO2019241157, WO2019217307, WO2020047192, WO2017087528, WO2018218070, WO2018218069, WO2018218071, WO2020027083, WO2020027084, WO2019215203, WO2019155399, WO2020035031, WO2014160200, WO2018195349, WO2018112240, WO2019204442, WO2019204449, WO2019104505, WO2016179558, WO2016176338, or related patents and applications, each of which is incorporated by reference in its entirety), (34) a SHC inhibitor (e.g., PP2, AID371185), (35) a GAB inhibitor (e.g., GAB-0001), (36) a GRB inhibitor, (37) a PI-3 kinase inhibitor (e.g., Idelalisib, Copanlisib, Duvelisib, Alpelisib, Taselisib, Perifosine, Buparlisib, Umbralisib, NVP-BEZ235-AN), (38) a MARPK inhibitor, (39) CDK4/6 (e.g., palbociclib, ribociclib, abemaciclib), or (40) MAPK inhibitor (e.g., VX-745, VX-702, RO-4402257, SCIO-469, BIRB-796, SD-0006, PH-797804, AMG-548, LY2228820, SB-681323, GW-856553, RWJ67657, BCT-197), or (41) an inhibitor of SHP pathway including SHP2 inhibitor (e.g., 6-(4-amino-4-methylpiperidin-1-yl)-3-(2,3-dichlorophenyl)pyrazin-2-amine, RMC-4630, ERAS-601,

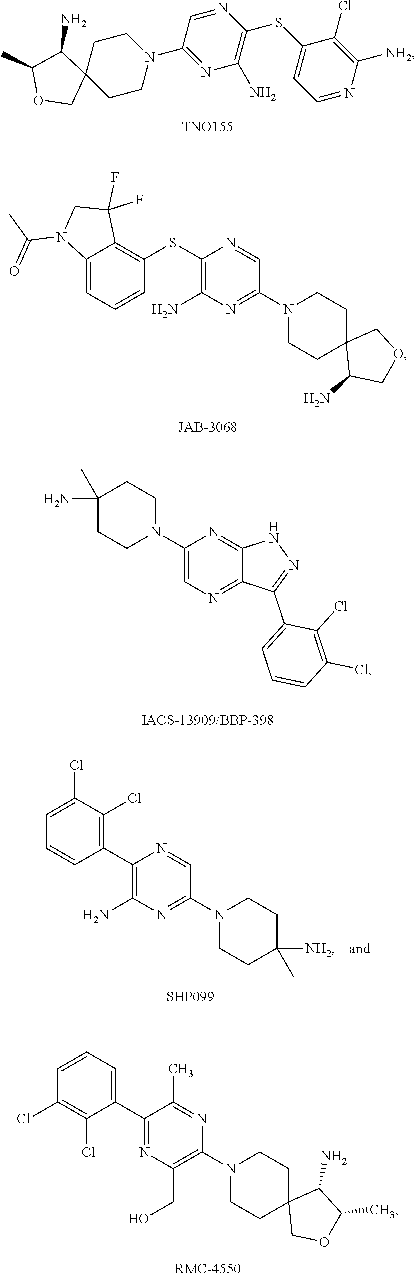

as well as SHP1 inhibitors; or (42) an inhibitor of a Kras mutant (e.g., Kras G12D including a compound described in WO2021041671, WO2021107160, WO2021091967, WO2021142252, WO2021150613, WO2021211864, WO2021118877, WO2021081212, WO2021108683, KRas G12C, KRas G12D, KRas G12S, KRas G12V, KRas G13D, KRas G13C, or KRas G13V). In some embodiments, any of the compounds herein that is capable of inhibiting a PTPN2 protein may be administered in combination or in conjunction with one or more checkpoint immune blockade agents (e.g., anti-PD-1 and/or anti-PD-L1 antibody, anti-CLTA-4 antibody).

In embodiments, a compound described herein may be administered in combination or conjunction with a SOS (e.g., SOS1) inhibitor, including a compound described in WO2021173524, WO2021203768, WO2020180770, WO2020180768, WO2021092115, WO2018172250, WO2019201848, WO2018115380, WO2019122129, or WO2021127429; all of which are herein incorporated by reference for any purpose.

In an aspect, the present disclosure provides a method of potentiating immunity of a subject in need thereof, comprising administering (e.g., systemically administering) a PTPN2 inhibitor (e.g., compound described herein) to the subject, thereby to potentiate immunity of the subject.

In another aspect, the present disclosure provides a method of potentiating immunity of a subject in need thereof, comprising (e.g., transiently) downregulating expression or activity of PTPN2 in vivo in a cell of the subject, thereby to potentiate immunity of the subject.

In another aspect, the present disclosure provides a method of potentiating immunity of a subject in need thereof, comprising (a) selecting the subject, wherein a cell of the subject exhibits expression or activity of PTPN2; and (b) downregulating the expression or activity of PTPN2 in a cell of the subject, thereby to potentiate immunity of the subject.

In another aspect, the present disclosure provides a method of potentiating immunity of a subject in need thereof, comprising (a) administering a lymphoid cell to the subject, wherein the lymphoid cell comprises (i) a chimeric T-cell receptor (TCR) sequence encoding a T-cell receptor fusion protein (TFP) and/or (ii) a chimeric antigen receptor (CAR) sequence encoding a CAR, wherein each of TFP and CAR exhibits specific binding to an antigen; and (b) separately administering a PTPN2 inhibitor (e.g., compound described herein) to the subject, thereby to potentiate immunity of the subject.

In another aspect, the present disclosure provides a method of potentiating immunity of a cell, comprising (a) contacting the cell with a PTPN2 inhibitor (e.g., compound described herein); and (b) introducing to the cell (i) a chimeric T-cell receptor (TCR) sequence encoding a T-cell receptor fusion protein (TFP) and/or (ii) a chimeric antigen receptor (CAR) sequence encoding a CAR, wherein each of TFP and CAR exhibits specific binding to an antigen, thereby to potentiate immunity of the cell, wherein (a) is performed prior to or concurrent with (b), thereby to potentiate immunity of the cell.

In another aspect, the present disclosure provides a method of increasing efficacy or reducing side effect of a cell therapy for a subject in need thereof, comprising (a) administering to the subject a cell comprising a chimeric antigen receptor (CAR) sequence encoding a CAR, wherein the CAR comprises an antigen-binding domain and an intracellular signaling domain, wherein the intracellular signaling domain is minimally required for activation of the CAR upon binding to an antigen; and (b) administering a PTNP2 inhibitor (e.g., compound described herein) to said subject prior to, concurrent with, or subsequent to (a).

In another aspect, the present disclosure provides a method of increasing efficacy or reducing side effect of a cell therapy for a subject in need thereof, comprising (a) administering to the subject a sub-therapeutic amount of a cell comprising a chimeric antigen receptor (CAR) sequence encoding a CAR, and (b) administering a PTNP2 inhibitor (e.g., compound described herein) to said subject prior to, concurrent with, or subsequent to (a).

In practicing any of the methods disclosed herein, a cell or a plurality of such cell may be administered (e.g., systemically administered) to the subject. In some cases, the cell may be a lymphoid cell that optically comprises (i) a chimeric T-cell receptor (TCR) sequence encoding a T-cell receptor fusion protein (TFP) and/or (ii) a chimeric antigen receptor (CAR) sequence encoding a CAR, wherein each of TFP and CAR exhibits specific binding to an antigen. In some cases, the cell may be administered (e.g., systemically administered) to the subject sequentially (e.g., prior to or subsequent to) or concurrent with administering (e.g., systemically administering) a PTPN2 inhibitor (e.g., compound described herein) to the subject. The cell may have been contacted previously with a PTPN2 inhibitor (e.g., compound described herein). Alternatively, the cell may not or need not be contacted with a PTPN2 inhibitor (e.g., compound described herein) prior to the administration of the cell to the subject.

In some embodiments, (i) the chimeric T-cell receptor sequence and/or (ii) the CAR sequence may be introduced to the cell directly (e.g., via a solution comprising (i) the chimeric T-cell receptor sequence and/or (ii) the CAR sequence), by chemical means (e.g., via one or more carriers such as liposomes for delivery of one or more nucleic acid sequences comprising (i) the chimeric T-cell receptor sequence and/or (ii) the CAR sequence), and/or viral means (e.g., when delivering one or more nucleic acid sequences comprising (i) the chimeric T-cell receptor sequence and/or (ii) the CAR sequence). For the viral means, the one or more nucleic acid sequence may in introduced in a chromosome of the cell, such as a nuclear chromosome and/or a mitochondrial chromosome. In other embodiments, the one or more nucleic acid sequence may not or need not be introduced in the chromosome of the cell, and as such be introduced to the cell as an epichromosomal molecule (e.g., a linear or circular nucleic acid molecule). In some embodiments, the cell may be a lymphoid cell.

Subsequent to the introduction, (i) the chimeric T-cell receptor sequence and/or (ii) the CAR sequence may persist in the cell for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 3 years, 4 years, 5 years, or more, or any time in between. Subsequent to the introduction, (i) the chimeric T-cell receptor sequence and/or (ii) the CAR sequence may persist in the cell for at most 5 years, 4 years, 3 years, 24 months, 23 months, 22 months, 21 months, 20 months, 19 months, 18 months, 17 months, 16 months, 15 months, 14 months, 13 months, 12 months, 11 months, 10 months, 9 months, 8 months, 7 months, 6 months, 5 months, 4 months, 3 months, 2 months, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less, or any time in between.

In some embodiments, introducing to the cell (i) the chimeric T-cell receptor sequence and/or (ii) the CAR sequence may be performed sequentially (e.g., prior to or subsequent to) or concurrent with contacting the cell with a PTPN2 inhibitor (e.g., compound described herein). When introduced sequentially, introducing (i) the chimeric T-cell receptor sequence and/or (ii) the CAR sequence and contacting with the PTPN2 inhibitor (e.g., compound described herein) may be performed by the same route (e.g. injections to the same location; tablets taken orally at the same time), or by a different route (e.g. a tablet taken orally while receiving an intravenous infusion). When introduced concurrently, for example, a first composition comprising (i) the chimeric T-cell receptor sequence and/or (ii) the CAR sequence and a second composition comprising the PTPN2 inhibitor (e.g., compound described herein) may be part of the same composition (e.g., the same condition media or a therapeutic regimen).

Contacting the cell with the PTPN2 inhibitor (e.g., compound described herein), whether systemically and/or transiently, as described in the present disclosure, may reduce PTPN2 signaling via reduction of PTPN2 activity or PTPN2 expression in the cell. For example, the cell can be cultured in a suitable medium, to which a PTPN2 inhibitor (e.g., compound described herein) is introduced for period of time sufficient to effect such reduction (or inhibition). Depending on the choice of the type of PTPN2 inhibitor (e.g., compound described herein), the contacting step may be effected by direct physical contact, pressure (e.g. by changing the shape of the cell via squeezing), chemical means (e.g., liposomes for delivery of nucleic acid based PTPN2 inhibitors), or viral means (e.g., when delivering shRNA, siRNA, or CRISPR-based PTPN2 inhibitors). The PTPN2 inhibitor (e.g., compound described herein) may directly be introduced to a subject lymphoid cell ex vivo or in vitro. In some embodiments, the cell can be in a subject, and the PTPN2 inhibitor (e.g., compound described herein) may be administered (e.g., systemically administered) to the subject to contact the cell in vivo. Upon such administration, at least a portion of the PTPN2 inhibitor (e.g., compound described herein) may contact a cell (e.g., a lymphoid cell, a cancer, or tumor cell, etc.) of the subject in vivo. A composition (e.g., a therapeutic regimen) comprising the PTPN2 inhibitor (e.g., compound described herein) may be administered to a target site comprising the cell (e.g., the cell may be part of the vascular or lymphatic system of the subject, or a localized tissue of interest or tumor). Alternatively or in addition to, the composition comprising the PTPN2 inhibitor (e.g., compound described herein) may be administered to a different site than the target site. Upon such administration, the PTPN2 inhibitor (e.g., compound described herein) may be directed to the target site or the cell via diffusion or via a medium such as a bodily fluid (e.g., blood).

When contacting a cell (e.g., a lymphoid cell) with the PTPN2 inhibitor (e.g., compound described herein) ex vivo, the cell may be treated with a composition (e.g., a solution) comprising the PTPN2 inhibitor (e.g., compound described herein) for at least 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 12 hours, 16 hours, 20 hours, 24 hours, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 2 months, 3 months, 4 months, 5 months, 6 months, or more, or any time in between. The cell may be treated with the composition comprising the PTPN2 inhibitor for at most 6 months, 5 months, 4 months, 3 months, 2 months, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 60 minutes, 50 minutes, 40 minutes, 30 minutes, 20 minutes, 10 minutes, 9 minutes, 8 minutes, 7 minutes, 6 minutes, 5 minutes, 4 minutes, 3 minutes, 2 minutes, 1 minute, or less, or any time in between. During the contacting period, the cell may be subjected to additional PTPN2 inhibitor (e.g., compound described herein) (e.g., to compensate for a limited half-life of the PTPN2 inhibitor (e.g., compound described herein) in culture media). Alternatively, during the contacting period, the cell may not be subjected to any additional PTPN2 inhibitor (e.g., compound described herein).

A process of contacting the cell with the PTPN2 inhibitor (e.g., compound described herein) (e.g., treating the cell with a composition comprising the PTPN2 inhibitor (e.g., compound described herein)) may be performed at least 1, 2, 3, 4, 5, or more times. In other embodiments, such process may be performed at most 5, 4, 3, 2, or 1 time.

In some embodiments, the cell as provided herein may retain expression or activity of PTPN2 prior to contacting (e.g., in vivo or ex vivo) the cell with the PTPN2 inhibitor (e.g., compound described herein). In some cases, any one of the methods disclosed herein may involve assessing the expression or activity of PTPN2 in the cell prior to contacting the cell with the PTPN2 inhibitor (e.g., compound described herein). In some examples, the cell may not exhibit any loss of the expression or activity of PTPN2, as compared to that present in a control sample, derived from e.g., another cell of the same origin of the cell or a progeny of the cell. In other examples, the cell may exhibit an expression or activity level of PTPN2 that is at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of that present in a control sample, derived from e.g., another cell of the same origin of the cell or a progeny of the cell. In yet some examples, the PTPN2 mRNA level, cDNA level, or PTPN2 polypeptide level expressed in the cell may be at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of that present in a control sample, derived from e.g., another cell of the same origin of the cell or a progeny of the cell. In other examples, the cell may exhibit an activity level of PTPN2 (e.g., a degree of dephosphorylation of a target substrate) that is at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of that present in a control sample, derived from e.g., another cell of the same origin of the cell or a progeny of the cell. In other examples, an amount of PTPN2-associated cfDNA or cfRNA level within a source of the cell (e.g., from a plasma of a subject from whom/which the cell was obtained or derived from) may be indicative of an expression level of PTPN2 in the cell. As such, the amount of PTPN2-associated cfDNA or cfRNA level within a source of the cell may be at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or more of that present in a control sample, e.g., another healthy subject who does not comprise or is not suspected of having a condition/disease of interest.

For any cell that is administered to a subject in need thereof, either with or without having been treated with a PTPN2 inhibitor (e.g., compound described herein), as provided in the present disclosure, the cell may be autologous or allogenic to the subject. The cell may have been obtained from the subject and treated ex vivo (e.g., contacting with the PTPN2 inhibitor (e.g., compound described herein), engineered to express (i) the TFG and/or (ii) the CAR, etc.) prior to the administration. Alternatively, the cell may be a progeny of a cell obtained from the subject, and the progeny may have been treated ex vivo (e.g., contacting with the PTPN2 inhibitor (e.g., compound described herein), engineered to express (i) the TFG and/or (ii) the CAR, etc.) prior to the administration. In a different alternative, the cell may be a progeny of a cell obtained from the subject, and the progeny may be administered to the subject without any engineering or modification thereof. In other embodiments, the cell may be heterologous to the subject. In some examples, the cell may be an allogeneic cell, derived from, e.g., another human subject.

Any one of the subject methods disclosed herein may further comprise administering a PTPN2 inhibitor (e.g., compound described herein) to the subject sequentially (e.g., prior to or subsequent to) or concurrent with administering a cell (e.g., a lymphoid cell) to the subject. In some embodiments, the cell may have been at least contacted previously with a PTPN2 inhibitor (e.g., compound described herein) and, optionally, express the TFP and/or the CAR. In other embodiments, the cell may not have been contacted previously with a PTPN2 inhibitor (e.g., compound described herein) and, optionally, express the TFP and/or the CAR. When introduced sequentially, the PTPN2 inhibitor (e.g., compound described herein) and the cell may be administered by the same route (e.g. injections to the same location; tablets taken orally at the same time), or separately by a different route (e.g. a tablet taken orally while receiving an intravenous infusion). When introduced concurrently, the PTPN2 inhibitor (e.g., compound described herein) and the cell may be, e.g., part of the same composition (e.g., the same condition media or a therapeutic regimen).

Any one of the subject methods disclosed herein may further comprise administering a lymphoid cell to the subject sequentially (e.g., prior to or subsequent to) or concurrent with administering a PTPN2 inhibitor (e.g., compound described herein) to the subject. The lymphoid cell may optionally comprise (i) the chimeric T-cell receptor sequence and/or (ii) the CAR sequence. When introduced sequentially, the PTPN2 inhibitor (e.g., compound described herein) and the lymphoid cell may be administered by the same route (e.g. injections to the same location; tablets taken orally at the same time), or by a different route (e.g. a tablet taken orally while receiving an intravenous infusion). When introduced concurrently, the PTPN2 inhibitor (e.g., compound described herein) and the cell may be, e.g., part of the same composition (e.g., the same condition media or a therapeutic regimen). As described elsewhere in the present disclosure, the subject being administered with a PTPN2 inhibitor (e.g., compound described herein) can retain, prior to the administration of the PTPN2 inhibitor, expression or activity of PTPN2 in the subject's cells, such as lymphoid cells (e.g., T cells, NK cells, HKGY cells, and B cells), cancer cells, or tumor cells.

In practicing any one of the methods disclosed herein, selecting the subject may be based on one or more thresholds of an expression or activity level of PTPN2 in the subject's cells, such as lymphoid cells including, without limitation, effector cells such as T cells, NK cells, HKGY cells, and B cells, cancer cells, or tumor cells. For example, the subject's lymphoid cells, cancer cells, or tumor cells exhibit a PTPN2 expression or activity level in his or her lymphoid cells, cancer cells, or tumor cells that is at least 30%, 40%, 50%, 60%, 70%, 80%, 90%, 95%, or more of that present in a control sample. In some examples, the PTPN2 mRNA level or cDNA level expressed in the subject's lymphoid cells, cancer cells, or tumor cells is at least 30%, 40%, 50%, 60%, 70%, 80%, 90%, 95%, or more of that present in a control sample. In some examples, the PTPN2 or PTPN2-associated cfDNA or cfRNA level from the subject's lymphoid cells, cancer cells, or tumor cells is at least 30%, 40%, 50%, 60%, 70%, 80%, 90%, 95%, or more of that present in a control sample. In some examples, the subject's lymphoid cells, cancer cells, or tumor cells carry two copies or least one copy of PTPN2 genomic DNA. In some examples, the PTPN2 polypeptide level expressed in the subject's lymphoid cells is at least 30%, 40%, 50%, 60%, 70%, 80%, 90%, 95%, or more of that present in a control sample. In some examples, the subject's lymphoid cells, cancer cells, or tumor cells exhibit a normal level of expression or activity of PTPN2 as compared to that of a control sample. In some cases, selecting the subject that exhibits expression or activity of PTPN2 results in a negative selection against subject that does not express or possess functional PTPN2 as PTPN2-null phenotype, such that the step of downregulating (e.g., transiently downregulating or permanently downregulating) expression or activity of PTPN2 will not be performed.

The control sample utilized in assessing the PTPN2 expression level can be a biological sample from a subject that does not exhibit a tumor or cancer, or from a subject that has not been diagnosed with a tumor or cancer and that has not been treated with a PTPN2 inhibitor (e.g., compound described herein). Such control sample can comprise PTPN2 polynucleotides or PTPN2 polypeptides from any of such subject's tissues or cells, including but not limited to such subject's lymphoid cells.

In some embodiments, downregulating (e.g., transiently downregulating or permanently downregulating) expression or activity of PTPN2 in the cell of the subject may be performed in vivo. In some cases, as described elsewhere in the present disclosure, the cell of the subject may be contacted by a PTPN2 inhibitor (e.g., compound described herein) in vivo by administering the PTPN2 inhibitor (e.g., compound described herein) to the subject comprising the cell. Administering a PTPN2 inhibitor (e.g., compound described herein) to a subject disclosed herein can stimulate or prolong anti-tumor or anti-cancer immunity. In other embodiments, downregulating expression or activity of PTPN2 in the cell of the subject may be performed in vivo. In some cases, as described elsewhere in the present disclosure, the cell of the subject may be isolated from the subject and may be contacted by a PTPN2 inhibitor (e.g., compound described herein) ex vivo, e.g., treated with a composition comprising the PTPN2 inhibitor (e.g., compound described herein).

In practicing any one of the methods disclosed herein, administering a cell (e.g., an autologous or allogeneic lymphoid cell that optionally expresses a TFP and/or a CAR) to the subject may be performed sequentially (e.g., prior to or subsequent to) or concurrent with downregulating (e.g., transiently downregulating or permanently downregulating) expression or activity of PTPN2 in the cell. In some embodiments, the downregulating may comprise introducing a PTPN2 inhibitor (e.g., compound described herein) to the cell, as provided in the present disclosure (e.g., contacting the cell with a PTPN2 inhibitor (e.g., compound described herein), or inducing the cell to express a PTPN2 inhibitor). When performed sequentially, a PTPN2 inhibitor (e.g., compound described herein) and the cell may be introduced to the subject by the same route (e.g. injections to the same location; tablets taken orally at the same time), or by a different route (e.g. a tablet taken orally while receiving an intravenous infusion). When performed concurrently, a PTPN2 inhibitor (e.g., compound described herein) and the cell may be, e.g., part of the same composition (e.g., the same condition media or a therapeutic regimen).

In some embodiments, a cell (e.g., a lymphoid cell, a cancer or tumor cell, etc.) of the subject may not exhibit a genetic alteration (e.g., mutation) of (i) a first gene encoding PTPN2 or (ii) a second gene operatively linked to PTPN2, wherein the genetic alteration reduces (or substantially inhibits) the expression and/or activity of PTPN2. In some examples, the second gene may be a promoter operatively linked to PTPN2 or an intron operatively linked to a gene product of PTPN2. Genetic alterations can include a mutation in a polynucleotide (e.g., DNA or RNA) encoding PTPN2 gene product. The mutation can affect any portion of the PTPN2 gene. The one or more PTPN2 mutations can include a mutation in the protein. The one or more PTPN2 mutations can be a point mutation, an insertion, a deletion, an amplification, a translocation, an inversion, or loss of heterozygosity. In some embodiments, the mutation is a loss of function. In some embodiments, the loss of function yields a dominant negative mutation. A mutation can be a frameshift mutation. A frameshift mutation can disrupt the reading frame, resulting in a completely different translated protein as compared to the original sequence. The mutation can be a nonsense mutation. The nonsense mutation can result in a premature stop codon, thus encoding a truncated, and possibly nonfunctional protein product. The PTPN2 mutation can be a nonsense mutation, wherein a single nucleotide alteration causes an amino acid substitution in the translated protein. The mutation can cause an alteration in one or more domain of the PTPN2 protein. The mutation can reduce binding efficacy of a PTPN2 protein with a PTPN2 substrate such as INSR, EGFR, CSF1R, PDGFR, JAK1, JAK2, JAK3, Src family kinases, STAT1, STAT3, STAT6, FYN, LCK, variations thereof, or combinations thereof. The mutation can reduce the ability of PTPN2 to dephosphorylate any one of the substrates disclosed herein, or reduce the ability of PTPN2 to interact with its upstream, or a downstream signaling molecules.

A method of potentiating immunity of a subject may comprise administering a lymphoid cell to the subject sequentially (e.g., prior to or subsequent to) and/or concurrent with the downregulation with the PTPN2 inhibitor (e.g., compound described herein). In some embodiments, contacting the lymphoid cell with a PTPN2 inhibitor (e.g., compound described herein) may be performed in vivo, e.g., via administration of the PTPN2 inhibitor (e.g., compound described herein) to the subject. In some cases, the subject may already comprise the lymphoid cell when the PTPN2 inhibitor (e.g., compound described herein) is administered to the subject. The lymphoid cell may be an endogenous cell of the subject. Alternatively, the lymphoid cell may be a heterologous lymphoid cell (e.g., an allogeneic cell from a donor or a xenograft cell). In other cases, the subject may not comprise the lymphoid cell when the PTPN2 inhibitor (e.g., compound described herein) is administered to the subject. Instead, the contact between the PTPN2 inhibitor and the lymphoid cell may occur upon administration of the lymphoid cell to the subject subsequent to the administration of the PTPN2 inhibitor to the subject. In some embodiments, contacting the lymphoid cell with a PTPN2 inhibitor (e.g., compound described herein) may be performed ex vivo, e.g., in an in vitro culture composition. The lymphoid cell of the subject may be subjected to ex vivo expansion (or cell proliferation) prior to, during, or subsequent to being contacted by the PTPN2 inhibitor (e.g., compound described herein). When the resulting lymphoid cell and/or a progeny thereof is administered to the subject, the lymphoid cell and/or the progeny thereof may be washed to be substantially free of the PTPN2 inhibitor (e.g., compound described herein). Alternatively, the lymphoid cell and/or the progeny may not or need not be washed to rid of any excess, used, or expressed PTPN2 inhibitor (e.g., compound described herein) prior to the administration to the subject.

In some embodiments, the method may further comprise introducing to the lymphoid cell (i) a chimeric T-cell receptor sequence encoding a T-cell receptor fusion protein (TFP) and/or (ii) a chimeric antigen receptor (CAR) sequence encoding a CAR, wherein each of TFP and CAR exhibits specific binding to an antigen. In some cases, the contacting of the lymphoid cell by the PTPN2 inhibitor (e.g., compound described herein) may be performed sequentially (e.g., prior to or subsequent to) or concurrent with the introducing to the lymphoid cell the chimeric T-cell receptor sequence and/or the CAR sequence. In some examples, the lymphoid cell may be contacted with a PTPN2 inhibitor (e.g., compound described herein) prior to being conditioned to express the TFP and/or the CAR. In other examples, the lymphoid cell may be contacted with a PTPN2 inhibitor while being conditioned to express the TFP and/or the CAR. In different examples, the lymphoid cell may be configured to express the TFP and/or the CAR prior to being contacted with a PTPN2 inhibitor (e.g., compound described herein).

In some embodiments, the downregulation of the expression or activity of PTPN2 in the lymphoid cell of the subject may be permanent. In other embodiments, as disclosed herein, the downregulation of the expression or activity of PTPN2 in a cell (e.g., the lymphoid cell of the subject) may comprise transiently downregulating the expression or activity of PTPN2.

In some cases, downregulating the expression or activity of PTPN2 in the lymphoid cell performed sequentially (e.g., prior to or subsequent to) or concurrent with the introducing to the lymphoid cell the chimeric T-cell receptor sequence and/or the CAR sequence. In some examples, the expression or activity of PTPN2 in the lymphoid cell may be downregulated (e.g., with a PTPN2 inhibitor) prior to being conditioned to express the TFP and/or the CAR. In other examples, the expression or activity of PTPN2 in the lymphoid cell may be downregulated (e.g., with a PTPN2 inhibitor) while being conditioned to express the TFP and/or the CAR. In different examples, the lymphoid cell may be configured to express the TFP and/or the CAR prior to downregulating the expression or activity of PTPN2 in the lymphoid cell (e.g., with a PTPN2 inhibitor).

In some embodiments, a CAR of the present disclosure contains a minimally required intracellular signaling domain capable of activating a signaling cascade (e.g., an immunoreceptor signaling cascade) of the cell (e.g., in a lymphoid cell) in comparison to a control cell that is (i) without the CAR and/or (ii) in absence of any CAR activation (e.g., in absence of any antigen of the antigen-binding domain of the CAR). A minimally required intracellular signaling domain of the CAR typically consists of a primary signaling domain and lacks a co-stimulatory signaling domain sequence or a functional co-stimulatory signaling domain, and hence exhibiting less potency in activating an immune signaling cascade as compared to one with the co-stimulatory signaling domain. In some examples, the CAR with a minimally required intracellular signaling domain is a first generation CAR. In some examples, the first generation CAR contains only a primary signaling domain selected from the group consisting of CD3zeta, CD28, 4-1BB, OX40, DAP10, ICOS, and a variant thereof. In some examples, the CAR with a minimally required intracellular signaling domain is a second generation CAR. In some examples, the second generation CAR contains only a primary signaling domain selected from the group consisting of CD3zeta, CD28, 4-1BB, OX40, DAP10, ICOS, and a variant thereof, and a co-stimulatory signaling domain that is a different member from the primary signaling domain. In some examples, a cell comprising the CAR with the minimally required intracellular signaling domain may induce a target activity of the cell of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, or more than that of a control cell. In some examples, a cell comprising the CAR with the minimally required intracellular signaling domain may induce a target activity of the cell of at most about 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1% or less than that of a control sample comprising a CAR with a more potent intracellular signaling domain. The more potent intracellular signaling domain may comprise a different polypeptide sequence (e.g., a polypeptide fragment derived from a different intracellular protein than the minimally required intracellular signaling domain) or an additional polypeptide sequence (e.g., the minimally required intracellular signaling domain plus one or more additional intracellular signaling domains). The additional polypeptide sequence may comprise at least 1, 2, 3, 4, 5, or more different intracellular signaling domains. Without wishing to be bound by theory, use of a CAR with the minimally required intracellular signaling domain may help to lower toxicity of a cell (e.g., a lymphocyte) expressing the CAR and/or increase persistence of the cell in the body of the subject in need of such cell therapy. In some cases, the use of PTPN2 inhibitor (e.g., compound described herein) in conjunction with CAR-T therapy obviates the need to use other CAR-T cell proliferation inhibitors to control the toxicities inherent in CAR-T therapy. Non-limiting CAR-T cell proliferation inhibitors are specific protein kinase inhibitors such as INSR, EGFR, CSF1R, PDGFR, JAK1, JAK2, JAK3, Src family kinases, STAT1, STAT3, STAT6, FYN, LCK, variations thereof, or combinations thereof. In some embodiments, the methods disclosed herein obviate the need to utilize Nintedanib, Dasatinib, Saracatinib, Ponatinib, Nilotinib, Danusertib, AT9283, Degrasyn, Bafetinib, KW-2449, NVP-BHG712, DCC-2036, GZD824, GNF-2, PD173955, GNF-5, Bosutinib, Gefitinib, Erlotinib, and/or Sunitinib in conjunction of a CAR-T therapy. Another advantage of using PTPN2 inhibitor (e.g., compound described herein) in conjunction with CAR-T therapy is that the amount of CAR-T cells required to yield a comparable level of in vivo efficacy is reduced. In some cases, a subtherapeutic amount of CAR-T cells is infused into a subject in need thereof. For example, one, two, or three orders of magnitude less of CAR-T cells are needed for treating a subject in need thereof. Where desired, less than 5×10⁶, 1×10⁶, 5×10⁵, 1×10⁵, 5×10⁴, 1×10⁴ CAR-T cells are needed to yield a comparable level of therapeutic effect as compared to a CAR-T therapy without the use of a PTPN2 inhibitor (e.g., compound described herein).

In practicing any one of the methods disclosed herein, examples of the target activity of the cell may include, but are not limited to, cytokine secretion, gene expression, cell proliferation, cytotoxicity against a target cell, cell death, chemotaxis, cellular metabolism, and/or cell exhaustion.

In practicing any one of the methods disclosed herein, a cell to be administered (e.g., systemically administered) may retain expression or activity of PTPN2 prior to administering a PTPN2 inhibitor (e.g., compound described herein) to the subject. In some examples, a PTPN2 inhibitor (e.g., compound described herein) may be administered to the subject prior to the administration of the cell, and the cell may be administered and contacted by the PTPN2 inhibitor (e.g., compound described herein) in vivo to effect downregulation (e.g., transient downregulation) of expression or activity of PTPN2 in the cell in vivo. In other examples, a PTPN2 inhibitor (e.g., compound described herein) and the cell may be administered at the same time, e.g., in a same composition or in different compositions, and the cell may be contacted by the PTPN2 inhibitor (e.g., compound described herein) ex vivo and/or in vivo to effect downregulation of expression or activity of PTPN2 in the cell. In different examples, a PTPN2 inhibitor (e.g., compound described herein) may be administered to the subject subsequent to the administration of the cell to the subject, and the cell may be contacted by the PTPN2 inhibitor (e.g., compound described herein) in vivo to effect downregulation of expression or activity of PTPN2 in the cell in vivo.

In practicing any one of the methods disclosed herein, a therapeutic amount or an effective amount may be an amount of a composition or a pharmaceutical formulation (e.g., a cell, a PTPN2 inhibitor (e.g., compound described herein), etc.) that is sufficient to elicit a desired response in the subject upon a treatment or method of the present disclosure. In some embodiments, a sub-therapeutic amount of a composition or a pharmaceutical formulation may be an amount of the composition or pharmaceutical formulation that is a fragment of the therapeutic amount. In some examples, a sub-therapeutic amount of a cell (e.g., a cell expression the CAR) may comprise a cell number that is at most 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less than a cell number of a therapeutic amount. For example, one, two, or three orders of magnitude less of CAR-T cells that are normally required absent of the use of PTPN2 inhibitor (e.g., compound described herein) are contemplated for administering into a subject in need thereof. Where desired, a sub-therapeutic amount of cells such as 5×10⁶, 1×10⁶, 5×10⁵, 1×10⁵, 5×10⁴, or 1×10⁴ CAR-T cells are needed to yield a comparable level of therapeutic effect as compared to a CAR-T therapy without the use of a PTPN2 inhibitor.

In some examples, a sub-therapeutic amount of a drug (e.g., a PTPN2 inhibitor (e.g., compound described herein)) may comprise a dose of the drug that is at most 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less than a dose of the drug of a therapeutic amount. Without wishing to be bound by theory, use of a sub-therapeutic amount (or dose) of a cell expressing the CAR may help to lower toxicity of such cell therapy and/or increase persistence of the cell in the body of the subject in need of such cell therapy.

In practicing any one of the methods disclosed herein, the immunity of a cell or a subject may be anti-tumor, anti-cancer activity, anti-viral infection activity, and/or anti-bacterial infection activity. In some embodiments, examples of a viral infection and bacterial infection may comprise human bacterial, human parasitic protozoan or human viral infections caused by microbial species including Plasmodium, Pneumocystis, herpes viruses (CMV, HSV 1, HSV 2, VZV, and the like), retroviruses, adenoviruses, and the like. In some examples, any one of the subject methods of the present disclosure may be used to treat or regulate HIV infections and related conditions such as tuberculosis, malaria, Pneumocystis pneumonia, CMV retinitis, AIDS, AIDS-related complex (ARC) and progressive generalized lymphadenopathy (PGL), and AIDS-related neurological conditions such as multiple sclerosis, and tropical spastic paraparesis. Other human retroviral infections that may be treated or regulated by any one of the subject methods of the present disclosure include Human T-cell Lymphotropic virus and HIV-2 infections.

In embodiments, when practicing any one of the methods disclosed herein, the PTPN2 inhibitor (e.g., compound described herein) does not regulate site-specific recombination of a gene encoding PTPN2. In some examples, the gene encoding PTPN2 or a gene operatively linked to the gene encoding PTPN2 (e.g., a transcription factor, an intron sequence, etc.) may not be flanked by a recombinase site (e.g., Cre recombinase or Flp recombinase substrates). In some examples, the PTPN2 inhibitor (e.g., compound described herein) may not be an activator of recombination of a recombinase site. In an example, the PTPN2 inhibitor (e.g., compound described herein) may not be an estrogen antagonist.

In practicing any one of the methods disclosed herein, the PTPN2 expression or activity level can be determined by detecting the PTPN2 polynucleotides or PTPN2 polypeptides present in a cell or tissue. A wide variety of nucleic acid assays are available for detecting and/or quantifying PTPN2 polynucleotides, including PTPN2 DNAs and PTPN2 RNAs. Exemplary nucleic acid assays include but are not limited to genotyping assays and sequencing methods. Sequencing methods can include next-generation sequencing, targeted sequencing, exome sequencing, whole genome sequencing, massively parallel sequencing, and the like.

Additional methods for assessing levels and/or concentration of PTPN2 polynucleotides in a tissue or a cell may include, but are not limited to, microarray hybridization assay, nucleic acid amplification assays including without limitation polymerase chain reaction (PCR), quantitative PCR (qPCR), real-time PCR (RT-PCR), digital PCR, and in situ sequencing (US20190024144, US20140349294, incorporated hereby by reference). Nucleic acid amplification can be linear or non-linear (e.g., exponential). Amplification may comprise directed changes in temperature, or may be isothermal. Conditions favorable to the amplification of target sequences by nucleic acid amplification assays are known in the art, can be optimized at a variety of steps in the process, and depend on characteristics of elements in the reaction, such as target type, target concentration, sequence length to be amplified, sequence of the target and/or one or more primers, primer length, primer concentration, polymerase used, reaction volume, ratio of one or more elements to one or more other elements, some or all of which can be altered.

In situ hybridization (ISH), RNase protection assay, and the like assays can also be employed for detecting PTPN2 polynucleotides and the expression level.

In some embodiments, the copy number PTPN2 gene is assessed by a method selected from the group consisting of in situ hybridization (ISH), Southern blot, immunohistochemistry (IHC), polymerase chain reaction (PCR), quantitative PCR (qPCR), quantitative real-time PCR (qRT-PCR), comparative genomic hybridization (CGH), microarray-based comparative genomic hybridization, and ligase chain reaction (LCR). In some embodiments, the in situ hybridization is selected from fluorescence in situ hybridization (FISH), chromogenic in situ hybridization (CISH) and silver in situ hybridization (SISH). In some embodiments, the copy number is assessed using a nucleic acid sample from the subject, such as genomic DNA, cDNA, ctDNA, cell-free DNA, RNA or mRNA.

PTPN2 expression and/or activity level can also be assessed by detecting and/or quantifying PTPN2 polypeptide level in a subject's tissue or cell. A variety of techniques are available in the art for protein analysis. They include but are not limited to immunohistochemistry (IHC), radioimmunoassays, ELISA (enzyme linked immunosorbent assays), “sandwich” immunoassays, immunoradiometric assays, in situ immunoassays (using e.g., colloidal gold, enzyme or radioisotope labels), western blot analysis, immunoprecipitation assays, immunofluorescent assays, flow cytometry, confocal microscopy, enzymatic assays, surface plasmon resonance and PAGE-SDS. One or more of these protein assays utilizes antibodies or fragments thereof that exhibits specific binding to PTPN2 polypeptides. A large number of anti-PTPN2 antibodies are available, including those provided by Invitrogen, Santa Cruz Biotechnology, OriGene Technologies, MilliporeSigma, Bio-Rad, Abcam, and Cell Signaling Technology.

In practicing any one of the subject methods as provided herein, the PTPN2 expression or activity, e.g., in a tumor tissue, a cancer cell, or a lymphoid cell, can be determined using any biological sample comprising the target cells (e.g., plasma cells or cells from a tumor site under investigation) or constituents thereof (e.g., constituents such as cfDNA from the plasma or the tumor site). The biological sample may be a solid or liquid biological sample from the subject under investigation or treatment. The biological sample may be a biopsy sample that is fixed, paraffin-embedded, fresh, or frozen. The biological sample may be obtained by any suitable means, including but not limited to needle aspiration, fine needle aspiration, core needle biopsy, vacuum assisted biopsy, large core biopsy, incisional biopsy, excisional biopsy, punch biopsy, shave biopsy, skin biopsy, and venipuncture.

The biological sample can be obtained from, without limitation, skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk, and/or other excretions or body tissues of the subject. In some embodiments, a selection of the biological sample may depend on the condition of the subject to be treated.

In some embodiments, a biological sample comprises cell-free DNA (cfDNA) derived from a whole blood or plasma of the subject. A sample may be analyzed directly for its contents, or may be processed to purify one or more of its contents for analysis. Methods of direct analysis of samples are known in the art and include, without limitation, mass spectrometry and histological staining procedures. In some embodiments, one or more components are purified from the sample for the detection of PTPN2 expression level or activity level. In some embodiments, the purified component of the biological sample is protein (e.g. total protein, cytoplasmic protein, or membrane protein). In some embodiments, the purified component of the sample is a nucleic acid, such as DNA (e.g. genomic DNA, cDNA, ctDNA, or cfDNA) or RNA (e.g. total RNA or mRNA).

In some embodiments, as abovementioned, the cell may be contacted by a PTPN2 inhibitor (e.g., compound described herein) in vivo by administering the PTPN2 inhibitor (e.g., compound described herein) to the subject comprising the cell. Administering a PTPN2 inhibitor (e.g., compound described herein) to a subject disclosed herein can stimulate or prolong anti-tumor or anti-cancer immunity. Not wishing to be bound by any particular theory, a PTPN2 inhibitor (e.g., compound described herein) reduces PTPN2 activity in a cell, leading to an augmented immunoreceptor signaling pathways, which in turn results in the activation of adaptive immunity against tumor or cancer cells.

Stimulation of anti-tumor or anti-cancer immunity can be established by any of the readout known in the art including without limitation: lymphoid cell proliferation (including proliferation of T cells such as CD4+ and/or CD8+ T cells, and clonal expansion other lymphoid cells), cytokine secretion, activation of effector function of lymphoid cells, reduction in T cell exhaustion, destabilization of regulatory T cells (Tregs) and/or their function, movement and/or trafficking of lymphoid cells, release of other intracellular signaling molecules, and phosphorylation of intracellular signaling molecules.

In some embodiments, anti-tumor immunity encompasses proliferation of the lymphoid cells including clonal expansion of the lymphoid cells that are capable of directly or indirectly mediating anti-tumor activity. Non-limiting examples of anti-tumor lymphoid cells are CD4+ and/or CD8+ T cells, NK cells, tumor infiltrating lymphocytes (TIL), especially those T cells capable of specific binding to one or more tumor antigens. Proliferation of the lymphoid cell can lead to a phenotypic change of the lymphoid cell. Treatment of a PTPN2 inhibitor (e.g., compound described herein) can stimulate or prolong lymphoid cell proliferation by about 1 fold, about 2 to about 5 fold, about 5 to about 10 fold, about 10 fold to about 50 fold, about 50 fold to about 100 fold or higher. Assessing lymphoid cell proliferation can be performed by a wide variety of assays known in the art, including without limitation, the use of cell staining, microscopy, flow cytometry, cell sorting, and combinations of these. A number of commercial kits for assessing various types of T cell or B cell proliferations are also suitable to assess the effect of PTPN2 inhibitor (e.g., compound described herein) on T cell or B cell proliferation (e.g., IncuCyte, CellTRrace Cell Proliferation Kits marketed by ThermoFisher). Proliferation can also be determined by phenotypic analysis of the lymphoid cells. For example, clumping of lymphoid cells in culture can signify proliferation of lymphoid cells as compared to comparable lymphoid cells without the treatment with a PTPN2 inhibitor (e.g., compound described herein).

In some embodiments, anti-tumor immunity stimulated or prolonged in response to a PTPN2 inhibitor (e.g., compound described herein) is evidenced by cytokine release from the lymphoid cells. Cytokine release by the lymphoid cell can comprise the release of IFNγ, TNFα, CSF, TGFβ, IL-1, IL-2, IL-4, IL-5, IL-6, IL-13, IL-17, IL-21, IL-22, granzyme, and the like. Lymphoid cells can generate about 1 fold, 2 fold, 3 fold, 4 fold, 5 fold, 6 fold, 7 fold, 8 fold, 9 fold, 10 fold, 50 fold, 100 fold or greater cytokine release in response to a PTPN2 inhibitor (e.g., compound described herein) treatment as compared to comparable lymphoid cells that are not being exposed to the PTPN2 inhibitor (e.g., compound described herein). Cytokine release may be determined and quantified using any immunoassays such as western blot, ELISA, flow cytometry, and the like.

In some embodiments, stimulated or prolonged anti-tumor immunity is evidenced by T cell activation. T cell activation can involve differential expression of antigen specific TCRs, certain cell surface markers and induction of cell proliferation signals. T cell activation may also involve stimulating its effector function including cytolytic activity against tumor or cancer cells, or helper activity including releasing cytokines. In some examples, T cells can be used to kill a tumor or cancer cell in vivo or in vitro in the presence of a PTPN2 inhibitor (e.g., compound described herein). Cell killing can be mediated by the release of one or more cytotoxic cytokines, for example IFNγ or granzyme, by the T cells. In some cases, a subject method can stimulate or prolong the (i) release of cytotoxins such as perforin, granzymes, and granulysin and/or (ii) induction of apoptosis via e.g., Fas-Fas ligand interaction between the T cells and a tumor or cancer cell, thereby triggering the destruction of the target cell. Cytotoxicity can be detected by staining, microscopy, flow cytometry, cell sorting, ELISPOT, chromium release cytotoxicity assay, and other cell death assays described in WO2011131472A1, which is incorporated herein by reference.

Cytotoxicity of a lymphoid cell can be greater in response to treating with a PTPN2 inhibitor (e.g., compound described herein) as compared to a comparable lymphoid cell lacking such treatment. A lymphoid cell treated with a PTPN2 inhibitor (e.g., compound described herein) can be about 5%, 10%, 15%, 20%, 25%, 30%, 35%, 40%, 45%, 50%, 55%, 60%, 65%, 70%, 75%, 80%, 85%, 90%, 95%, 100%, 500% or more cytotoxic against tumor or cancer cells as compared to a comparable lymphoid cell lacking the treatment. In some embodiments, a change in cytotoxicity can comprise comparing such activity before and after treating the lymphoid cell with a PTPN2 inhibitor (e.g., compound described herein).

In some examples, a reduction in expression or activity of such markers including PD1, Foxp3, or FoxO3a is indicative of Treg destabilization, and hence an enhanced anti-tumor immunity. In addition, Treg destabilization, as reflected by a decreased T cell exhaustion, can be demonstrated by an enhanced cytokine release, e.g., release of IL-2, IFNγ, TNF and other chemokines.

Anti-tumor immunity can also be evidenced by movement and/or trafficking of the lymphoid cells in response to a treatment with a PTPN2 inhibitor (e.g., compound described herein). In some embodiments, movement can be determined by quantifying localization of the lymphoid cell to a target site such as a tumor tissue. For example, lymphoid cells can be quantified at the target before or after administration of a PTPN2 inhibitor (e.g., compound described herein). Quantification can be performed by isolating a lesion and quantifying a number of lymphoid cells, for example tumor infiltrating lymphocytes. Movement and/or trafficking of lymphoid cells in a tumor tissue after administering a PTPN2 inhibitor (e.g., compound described herein) can be greater than that of a control lacking the administration of a PTPN2 inhibitor (e.g., compound described herein). In some embodiments, the number of lymphoid cells accumulated at the tumor tissue of interest can be about 1 fold, 5 fold, 10 fold, 15 fold, 50 fold, 100 fold or greater than that of a control not being treated with a PTPN2 inhibitor (e.g., compound described herein). Trafficking can also be determined in vitro utilizing a transwell migration assay. In some embodiments, the number of lymphoid cells administered with a PTPN2 inhibitor (e.g., compound described herein) exhibits about 1 fold, 5 fold, 10 fold, 15 fold, 50 fold, 100 fold or greater as compared to that of control lymphoid cells not being administered with a PTPN2 inhibitor (e.g., compound described herein).

Stimulating and/or prolonging anti-tumor immunity in a subject can also be assessed by one or more (in any combination) of the foregoing results, although alternative or additional results of the referenced tests and/or other tests can evidence such desired outcome. In some embodiments, anti-tumor immunity is considered stimulated if there exists at least about 10%, 20%, 30%, 40%, 50%, 60%, 70%, 75%, 80%, 90%, 95%, 100%, 110%, 120%, 150%, 200%, 300%, 400%, 500%, 600%, 700%, 1000%, 10000% or more improvement, using an appropriate measure (e.g. tumor size reduction, duration of tumor size stability, duration of time free from metastatic events, duration of disease-free survival). Improved immunity may also be expressed as fold improvement, such as at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or more, using an appropriate measure (e.g. tumor size reduction, duration of tumor size stability, duration of time free from metastatic events, duration of disease-free survival).

A number of secondary parameters can be employed to determine stimulated and/or prolonged anti-tumor immunity. Examples of secondary parameters include, but are not limited to, the lack of new tumors, a reduction of circulating tumor antigens or markers (e.g., CEA, PSA, CA-125, or cfDNA, ctDNA), the lack of detectable cancer cell or tumor marker by way of biopsy, surgical downstaging (i.e., conversion of the surgical stage of a tumor from unresectable to resectable), MRI, ultrasound, PET scans and any other detection means, all of which can point to the overall immunity to tumor or cancer in a subject. Examples of tumor markers and tumor-associated antigens that can be evaluated as indicators of improved immunity include, but are not limited to, carcinembryonic antigen (CEA) prostate-specific antigen (PSA), CA-125, CA19-9, ganglioside molecules (e.g., GM2, GD2, and GD3), MART-1, heat shock proteins (e.g., gp96), sialyl Tn (STn), tyrosinase, MUC-1, HER-2/neu, c-erb-B2, KSA, PSMA, p53, RAS, EGF-R, VEGF, MAGE, gp100, Ki-67, STK15, Survivin, Cyclin B1, Stromelysin, Cathepsin L2, 3MYBL2, and any ctDNA known in the art. BMC Med. 16:166, 2018.

In some embodiments, prolonged immunity is evidenced by tumor being stabilized (e.g., one or more tumors do not increase more than 1%, 5%, 10%, 15%, or 20% in size, and/or do not metastasize) as a result of treatment with a PTPN2 inhibitor (e.g., compound described herein). In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor or the number of tumor cells is reduced by at least about 5%, 10%, 15%, 20%, 25, 30%, 35%, 40%, 45%, 50%, 55%, 60%, 65%, 70%, 75%, 80%, 85%, 90%, 95%, or more. In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

The methods disclosed herein can be applied to treat, stimulate and/or or prolong immunity against a wide variety of cancers, including both solid tumor hematological cancers. For example, the subject methods can be applied to: Acute Lymphoblastic Leukemia (ALL), Acute Myeloid Leukemia (AML), Adrenocortical Carcinoma, Childhood Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Childhood (Brain Cancer), A typical Teratoid/Rhabdoid Tumor, Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma—see Non-Hodgkin Lymphoma, Carcinoid Tumor (Gastrointestinal), Childhood Carcinoid Tumors, Cardiac (Heart) Tumors, Atypical Teratoid/Rhabdoid Tumor, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma (Mycosis Fungoides and Sëzary Syndrome), Ductal Carcinoma In Situ (DCIS), Embryonal Tumors, Endometrial Cancer (Uterine Cancer), Ependymoma, Esophageal Cancer, Esthesioneuroblastoma(Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Langerhans Cell Hodgkin Lymphoma, Hypopharyngeal Cancer (Head and Neck Cancer), Islet Cell Tumors, Pancreatic Neuroendocrine Tumors, Kaposi Sarcoma(Soft Tissue Sarcoma), Kidney (Renal Cell) Cancer, Laryngeal Cancer (Head and Neck Cancer), Leukemia, Lip and Oral Cavity Cancer(Head and Neck Cancer), Liver Cancer, Lung Cancer (e.g., Non-Small Cell and Small Cell), Lymphoma, Male Breast Cancer, Malignant Fibrous Histiocytoma of Bone and Osteosarcoma, Melanoma, Merkel Cell Carcinoma(Skin Cancer), Mesothelioma, Malignant, Metastatic Cancer, Metastatic Squamous Neck Cancer with Occult Primary (Head and Neck Cancer), Midline Tract Carcinoma, Mouth Cancer(Head and Neck Cancer), Multiple Endocrine Neoplasia, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, CML, Myeloid Leukemia, Acute (AML), Myeloproliferative Neoplasms, Chronic, Nasal Cavity and Paranasal Sinus Cancer(Head and Neck Cancer), Nasopharyngeal Cancer(Head and Neck Cancer), Neuroblastoma, Non-Hodgkin Lymphoma, Non-Small Cell Lung Cancer, Oral Cancer, Lip and Oral Cavity Cancer and Oropharyngeal Cancer(Head and Neck Cancer), Osteosarcoma and Malignant Fibrous Histiocytoma of Bone, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Rectal Cancer, Retinoblastoma, Rhabdomyosarcoma, Salivary Gland Cancer (Head and Neck Cancer), Sarcoma, Childhood Rhabdomyosarcoma(Soft Tissue Sarcoma), Childhood Vascular Tumors (Soft Tissue Sarcoma), Ewing Sarcoma(Bone Cancer), Kaposi Sarcoma (Soft Tissue Sarcoma), Osteosarcoma(Bone Cancer), Soft Tissue Sarcoma, Uterine Sarcoma, Sëzary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, T-Cell Lymphoma, Cutaneous, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter (Kidney (Renal Cell) Cancer), Ureter and Renal Pelvis, Transitional Cell Cancer (Kidney (Renal Cell) Cancer, Urethral Cancer, Uterine Cancer, Endometrial, Uterine Sarcoma, Vaginal Cancer, Vascular Tumors (Soft Tissue Sarcoma), Vulvar Cancer, and Wilms Tumor and Other Childhood Kidney Tumors, and any of the aforementioned cancers exhibiting expression and/or activity of PTPN2 in the cancer cells.

Certain embodiments contemplate a human subject that has been diagnosed with a cancer, such as one in which PTPN2 expression or activity is detectable (e.g., aberrantly low, normal, or high) in the cancer cells or tumor tissue. Certain other embodiments contemplate a non-human subject, for example a non-human primate such as a macaque, chimpanzee, gorilla, vervet, orangutan, baboon or other non-human primate, including such non-human subjects that can be known to the art as preclinical models, the tumor tissue or cancer cells of which exhibit expression and/or activity of PTPN2. Certain other embodiments contemplate a non-human subject that is a mammal, for example, a mouse, rat, rabbit, pig, sheep, horse, bovine, goat, gerbil, hamster, guinea pig or other mammal. There are also contemplated other embodiments in which the subject or biological source can be a non-mammalian vertebrate, for example, another higher vertebrate, or an avian, amphibian or reptilian species, or another subject or biological source. In certain embodiments of the present disclosure, a transgenic animal is utilized. A transgenic animal is a non-human animal in which one or more of the cells of the animal include a nucleic acid that is non-endogenous (i.e., heterologous) and is present as an extrachromosomal element in a portion of its cell or stably integrated into its germ line DNA (i.e., in the genomic sequence of most or all of its cells).

Where desired, the subject can be screened for the presence of expression or activity of PTPN2 in the subject's tumor or cancer cells. The subject can also be screened for the retention of PTPN2 expression and/or activity in one or more types of subject's lymphoid cells. Screening for the presence or the absence of expression or activity of PTPN2 can be carried out by analyzing the PTPN2 polynucleotide or PTPN2 polypeptide with any of the nucleic acid or protein assays disclosed herein. One or more of the screening steps can be performed concurrent with, subsequent to, or more likely, prior to administering a PTPN2 inhibitor to the subject.

The present disclosure also provides a cell (including a population of cells, such as a population of lymphoid cells) modified to express an exogenous sequence, and wherein expression and/or activity of PTPN2 in said cell has been inhibited (including reduction and elimination). In one aspect, provided in the disclosure is a lymphoid cell in which the expression and/or function of PTPN2 in said cell is inhibited. Such inhibition can be transient or permanent, occurring in vitro, ex vivo, or in vitro. In some cases, as used herein, inhibiting expression and/or function of a target molecule may be referred to downregulation of expression and/or function of the target molecule. A modified lymphoid cell of the present disclosure can be further characterized in that it comprises: (a) a chimeric T-cell receptor sequence encoding a T-cell receptor fusion protein (TFP), and/or (b) a chimeric antigen receptor (CAR) sequence encoding a CAR, wherein each of TFP and CAR exhibits specific binding to an antigen, including but not limited to a tumor or tumor-associated antigen.

Not wishing to be bound by any particular theory, inhibiting PTPN2 expression and/or activity of such lymphoid cell can lead to an augmented immunoreceptor signaling, which in turn results in the activation of an adaptive immunity against tumor or cancer cells. When its PTPN2 expression or activity is inhibited, the modified lymphoid cells can exhibit enhanced cell proliferation (including proliferation of T cells such as CD4+ and/or CD8+ T cells, and clonal expansion other lymphoid cells), enhanced cell activity (including e.g., cytokine secretion, activation of effector function, trafficking to tumor site or cancer cell), or enhanced disability (e.g., reduction in T cell exhaustion, destabilization of regulatory T cells (Tregs) in terms of cell number and cellular function).

In practicing any one of the methods disclosed herein, a subject cell (e.g., a modified cell such as a modified lymphoid cell) may comprise an enhancer moiety capable of enhancing one or more activities of the cell. In some embodiments, an enhancer moiety suitable for incorporating into a subject cell (e.g., a modified lymphoid cell) can be cytokines and growth factors capable of stimulating the growth, clonal expansion, and/or enhancing persistence of the immune cell in vivo. An enhancer may be intracellular, membrane-bound (e.g., a receptor or an adaptor protein of a receptor), or secreted by the cell. Encompassed are enhancer moieties selected from the group consisting of IL-2, IL-3, IL-4, IL-6, IL-7, IL-10, IL-11, IL-12, IL-15, IL-17, IL-18, IL-21, IL-23, PD-1, PD-L1, CD122, CSF1R, CTAL-4, TIM-3, TGFR beta, receptors for the same, functional fragments thereof, functional variants thereof, and combinations thereof. An enhancer moiety may be expressed from an endogenous gene of the cell. Alternatively or in addition to, an enhancer moiety may be expressed from a heterologous gene introduced to the cell. Such heterologous gene may be chromosomal (e.g., in the nuclear chromosome or mitochondrial chromosome) or epichromosomal. In some examples, a cell (e.g., a modified immune cell configured to express a TFP and/or a CAR) may be engineered such that one or more enhancer moieties are constitutively expressed and/or activated. In other examples, the one or more enhancer moieties may be transiently expressed for a limited time. In different examples, the one or more enhancer moieties may be conditionally expressed under, e.g., activation of a cellular signaling.

In practicing any one of the methods disclosed herein, a subject cell (e.g., a modified cell such as a modified lymphoid cell) may comprise an inducible cell death moiety, which inducible cell death moiety effects cell death (e.g., suicide) of the cell upon contact with a cell death activator. Where desired, an inducible cell death moiety is selected from the group consisting of: caspase-1 ICE, caspase-3 YAMA, inducible Caspase 9 (iCasp9), AP1903, HSV-TK, CD19, RQR8, tBID, CD20, truncated EGFR, Fas, FKBP12, CID-binding domain (CBD), and any combination thereof. Examples of further suicide systems include those described by Jones et al. (Jones B S, Lamb L S, Goldman F and Di Stasi A (2014) Improving the safety of cell therapy products by suicide gene transfer. Front. Pharmacol. 5:254. doi: 10.3389/fphar.2014.00254), which is incorporated herein by reference in its entirety. Where desired, a suitable inducible cell death moiety can be HSV-TK, and the cell death activator is GCV. Where further desired, a suitable inducible cell death moiety can be iCasp9, and the cell death activator is AP1903.

A TFP comprised in the subject lymphoid cell typically comprises a TCR subunit comprising (1) a TCR extracellular domain capable of specific binding to an antigen domain, and (2) an intracellular signaling domain. Upon expression of the TFP, it forms a T cell receptor (TCR) complex.

In some embodiments, the TCR extracellular domain comprises (1) an antigen binding domain capable of specific binding to the antigen, and (2) an extracellular domain or portion thereof of a protein including, e.g., the alpha, beta or zeta chain of the T-cell receptor, or CD3 epsilon, CD3 gamma, or CD3 delta, or in alternative embodiments, CD28, CD45, CD4, CD5, CD8, CD9, CD16, CD22, CD33, CD37, CD64, CD80, CD86, CD134, CD137, CD154. In general, the antigen binding domain and the extracellular domain are operatively linked together, e.g., in the same reading frame.

In some embodiments, a subject CAR comprises an antigen-binding domain and an intracellular signaling domain. In some examples, the antigen-binding domain and the intracellular signaling domain of the CAR are linked via a transmembrane domain. Antigen Binding Domain of TFP or CAR

The Antigen Binding Domain of a TFP or CAR Disclosed Herein Typically Comprises an Antigen-Specific Binding Element, the choice of which depends upon the type and number of antigen of interest. For example, the antigen binding domain may be chosen to recognize a cell surface marker on a target cell associated with a particular disease state. Non-limiting examples of cell surface markers include those associated tumor or cancer, with viral, bacterial and parasitic infections, autoimmune disease, inflammation diseases and metabolic disease. Cell surface markers can include, without limitation, carbohydrates, glycolipids, glycoproteins; CD (cluster of differentiation) antigens present on cells of a hematopoietic lineage (e.g., CD2, CD4, CD8, CD21, etc.), γ-glutamyltranspeptidase, an adhesion protein (e.g., ICAM-1, ICAM-2, ELAM-1, VCAM-1), hormone, growth factor, cytokine, and other ligand receptors, ion channels, and the membrane-bound form of an immunoglobulin μ chain.

Of particular interest are biological markers associated with a tumor or cancer or a stage or state of a cancer. A vast variety of disease-related biological markers have been identified, and the corresponding targeting moieties have been generated, including but not limited to cancer antigen-50 (CA-50), cancer antigen-125 (CA-125) associated with ovarian cancer, cancer antigen 15-3 (CA15-3) associated with breast cancer, cancer antigen-19 (CA-19) and cancer antigen-242 associated with gastrointestinal cancers, carcinoembryonic antigen (CEA), carcinoma associated antigen (CAA), chromogranin A, epithelial mucin antigen (MC5), human epithelium specific antigen (HEA), Lewis(a)antigen, melanoma antigen, melanoma associated antigens 100, 25, and 150, mucin-like carcinoma-associated antigen, multidrug resistance related protein (MRPm6), multidrug resistance related protein (MRP41), Neu oncogene protein (C-erbB-2), neuron specific enolase (NSE), P-glycoprotein (mdrl gene product), multidrug-resistance-related antigen, p170, multidrug-resistance-related antigen, prostate specific antigen (PSA), CD56, and NCAM.

In some examples, the antigen binding domain of the subject TCR specifically binds to CD19. A large number of exemplary anti-CD19 antigen binding domains and constructs thereof are described in U.S. Pat. Nos. 8,399,645; 7,446,190; WO2012/079000; WO2014/031687; U.S. Pat. No. 7,446,190; each of which is herein incorporated by reference in its entirety.

In some other examples, the antigen binding domain of the subject TCR specifically binds to BCMA. Exemplary anti-BCMA antigen binding domains and constructs thereof are described in e.g., WO2012163805, WO200112812, and WO2003062401, WO2016/014565, WO2014/122144, WO2016/014789, WO2014/089335, WO2014/140248, each of which is hereby incorporated by reference in its entirety.

In some other examples, the antigen binding domain of the subject TCR specifically binds to CD123. Exemplary anti-CD123 antigen binding domains and constructs thereof are described in e.g., WO2014/130635, WO2016/028896, WO2008/127735, WO2014/138805, WO2014/138819, WO2013/173820, WO2014/144622, WO2001/66139, WO2010/126066, WO2014/144622, and US2009/0252742, each of which is incorporated herein by reference in its entirety.

In yet some other examples, the antigen binding domain of the subject TCR specifically binds to CD38, exemplary anti-CD38 antigen binding domains are embodied in daratumumab (described in e.g., Groen et al., Blood 116(21):1261-1262 (2010); MOR202 (see, e.g., U.S. Pat. No. 8,263,746); or antibodies described in U.S. Pat. No. 8,362,211.

In some other examples, the antigen binding domain of the subject TCR specifically binds to Tn antigen. Exemplary anti-Tn antigen binding domains and constructs thereof are described in e.g., US 2014/0178365, U.S. Pat. No. 8,440,798, Brooks et al., PNAS 107(22):10056-10061 (2010), and Stone et al., Oncolmmunology 1(6):863-873 (2012). In yet some other examples, the antigen binding domain of the subject TCR specifically binds to CS-1. Exemplary anti-CS-1 antigen binding domains and constructs thereof are described in Elotuzumab (BMS), see e.g., Tai et al., 2008, Blood 112(4):1329-37; Tai et al., 2007, Blood. 110(5):1656-63. In yet some other examples, the antigen binding domain of the subject TCR specifically binds to mesothelin. Exemplary anti-mesothelin antigen binding domain are described in, e.g., WO2015/090230, WO1997/025068, WO1999/028471, WO2005/014652, WO2006/099141, WO2009/045957, WO2009/068204, WO2013/142034, WO2013/040557, WO2013/063419, each of which is incorporated by reference in its entirety. In yet some other examples, the antigen binding domain of the subject TCR specifically binds to CD22, exemplary anti-CD22 antigen binding domains are described in Haso et al., Blood, 121(7): 1165-1174 (2013); Wayne et al., Clin Cancer Res 16(6): 1894-1903 (2010), each of which is incorporated herein by reference. In yet some other examples, the antigen binding domain of the subject TCR specifically binds to CLL-1, exemplary anti- CLL-1 antigen binding domains are described in WO2016/014535, incorporated herein by reference.

In yet some other examples, the antigen binding domain of the subject TCR specifically binds to CD33, exemplary anti-CD33 antigen binding domains are described in WO2016/014576 and WO2016/014576, each of which is incorporated by reference in its entirety. In yet some other examples, the antigen binding domain of the subject TCR specifically binds to GD2, exemplary anti-GD2 antigen binding domains are described in WO2012033885, WO2013040371, WO2013192294, WO2013061273, WO2013123061, WO2013074916, WO201385552, WO 2011160119, and US 20100150910, each of which is incorporated by reference in its entirety. In yet some other examples, the antigen binding domain of the subject TCR specifically binds to PSMA, exemplary anti- PSMA antigen binding domains are described in US 20110268656 (J591 ScFv); WO 2006125481 (mAbs 3/A12, 3/E7 and 3/F11) and single chain antibody fragments (scFv A5 and D7), each of which is incorporated by reference in its entirety. In yet some other examples, the antigen binding domain of the subject TCR specifically binds to FLT3, exemplary anti- FLT3 antigen binding domains are described in e.g., WO2011076922, U.S. Pat. No. 5,777,084, EP0754230, US20090297529, and several commercial catalog antibodies (R&D, ebiosciences, Abcam), each of which is incorporated by reference in its entirety. In yet some other examples, the antigen binding domain of the subject TCR specifically binds to ROR1, exemplary anti- ROR1 antigen binding domains are described in WO 2011159847, US20130101607, each of which is incorporated by reference in its entirety. In yet some other examples, the antigen binding domain of the subject TCR specifically binds to TAG72, exemplary anti-TAG72 antigen binding domains are described in Hombach et al., Gastroenterology 113(4):1163-1170 (1997); and Abcam ab691.

In yet some other examples, the antigen binding domain of the subject TCR specifically binds to FAP, exemplary anti- FAP antigen binding domains are described in US 2009/0304718, incorporated herein by reference. In yet some other examples, the antigen binding domain of the subject TCR specifically binds to CD44v6, exemplary anti- CD44v6 antigen binding domains are described in Casucci et al., Blood 122(20):3461-3472 (2013). In yet some other examples, an antigen binding domain against CEA is an antigen binding portion, e.g., CDRs, of an antibody described in, e.g., Chmielewski et al., Gastoenterology 143(4):1095-1107 (2012). In yet some other examples, an antigen binding domain against EPCAM is an antigen binding portion, e.g., CDRS, of an antibody selected from MT110, EpCAM-CD3 bispecific Ab (see, e.g., clinicaltrials.gov/ct2/show/NCT00635596); Edrecolomab; 3622W94; ING-1; and adecatumumab (MT201). In yet some other examples, an antigen binding domain against PRSS21 is an antigen binding portion, e.g., CDRs, of an antibody described in U.S. Pat. No. 8,080,650. In yet some other examples, an antigen binding domain against IL-13Ra2 is an antigen binding portion, e.g., CDRs, of an antibody described in, e.g., WO2008/146911, WO2004087758, several commercial catalog antibodies, and WO2004087758. In yet some other examples, an antigen binding domain against B7H3 is an antigen binding portion, e.g., CDRs, of an antibody MGA271 (Macrogenics). In yet some other examples, an antigen binding domain against KIT is an antigen binding portion, e.g., CDRs, of an antibody described in, e.g., U.S. Pat. No. 7,915,391, US20120288506, and several commercial catalog antibodies. In yet some other examples, an antigen binding domain against CD30 is an antigen binding portion, e.g., CDRs, of an antibody described in, e.g., U.S. Pat. No. 7,090,843 B1, and EP0805871. In yet some other examples, an antigen binding domain against GD3 is an antigen binding portion, e.g., CDRs, of an antibody described in, e.g., U.S. Pat. Nos. 7,253,263; 8,207,308; US 20120276046; EP1013761; WO2005035577; and U.S. Pat. No. 6,437,098. In yet some other examples, an antigen binding domain against CD171 is an antigen binding portion, e.g., CDRs, of an antibody described in, e.g., Hong et al., J Immunother 37(2):93-104 (2014). In yet some other examples, an antigen binding domain against IL-11Ra is an antigen binding portion, e.g., CDRs, of an antibody available from Abcam (cat #ab55262) or Novus Biologicals (cat #EPR5446). In another embodiment, an antigen binding domain again IL-11Ra is a peptide, see, e.g., Huang et al., Cancer Res 72(1):271-281 (2012). In yet some other examples, an antigen binding domain against PSCA is an antigen binding portion, e.g., CDRs, of an antibody described in, e.g., Morgenroth et al., Prostate 67(10):1121-1131 (2007) (scFv 7F5); Nejatollahi et al., J of Oncology 2013 (2013), article ID 839831 (scFv C5-II); and US Pat Publication No. 20090311181. In yet some other examples, an antigen binding domain against VEGFR2 is an antigen binding portion, e.g., CDRs, of an antibody described in, e.g., Chinnasamy et al., J Clin Invest 120(11):3953-3968 (2010). In yet some other examples, an antigen binding domain against LewisY is an antigen binding portion, e.g., CDRs, of an antibody described in, e.g., Kelly et al., Cancer Biother Radiopharm 23(4):411-423 (2008) (hu3S193 Ab (scFvs)); Dolezal et al., Protein Engineering 16(1):47-56 (2003) (NC10 scFv). In yet some other examples, an antigen binding domain against CD24 is an antigen binding portion, e.g., CDRs, of an antibody described in, e.g., Maliar et al., Gastroenterology 143(5):1375-1384 (2012). In yet some other examples, an antigen binding domain against CD20 is an antigen binding portion, e.g., CDRs, of the antibody Rituximab, Ofatumumab, Ocrelizumab, Veltuzumab, or GA101. In yet some other examples, an antigen binding domain against PDGFR-beta is an antigen binding portion, e.g., CDRs, of an antibody Abcam ab32570. In yet some other examples, an antigen binding domain against SSEA-4 is an antigen binding portion, e.g., CDRs, of antibody MC813 (Cell Signaling), or other commercially available antibodies. In yet some other examples, an antigen binding domain against Folate receptor alpha is an antigen binding portion, e.g., CDRs, of the antibody IMGN853, or an antibody described in US20120009181; U.S. Pat. No. 4,851,332, LK26: U.S. Pat. No. 5,952,484. In yet some other examples, an antigen binding domain against ERBB2 (Her2/neu) is an antigen binding portion, e.g., CDRs, of the antibody trastuzumab, or pertuzumab. In yet some other examples, an antigen binding domain against MUC1 is an antigen binding portion, e.g., CDRs, of the antibody SAR566658. In yet some other examples, the antigen binding domain against EGFR is antigen binding portion, e.g., CDRs, of the antibody cetuximab, panitumumab, zalutumumab, nimotuzumab, or matuzumab. In one embodiment, the antigen binding domain against EGFRvIII is or may be derived from an antigen binding domain, e.g., CDRs, scFv, or VH and VL, of an antibody, antigen-binding fragment or CAR described in, e.g., PCT publication WO2014/130657 (In one embodiment the CAR is a CAR described in WO2014/130657, the contents of which are incorporated herein in their entirety). In yet some other examples, an antigen binding domain against NCAM is an antigen binding portion, e.g., CDRs, of the antibody clone 2-2B: MAB5324 (EMD Millipore). In yet some other examples, an antigen binding domain against Ephrin B2 is an antigen binding portion, e.g., CDRs, of an antibody described in, e.g., Abengozar et al., Blood 119(19):4565-4576 (2012). In yet some other examples, an antigen binding domain against IGF-I receptor is an antigen binding portion, e.g., CDRs, of an antibody described in, e.g., U.S. Pat. No. 8,344,112 B2; EP2322550 A1; WO 2006/138315, or PCT/US2006/022995. In yet some other examples, an antigen binding domain against CAIX is an antigen binding portion, e.g., CDRs, of the antibody clone 303123 (R&D Systems). In yet some other examples, an antigen binding domain against LMP2 is an antigen binding portion, e.g., CDRs, of an antibody described in, e.g., U.S. Pat. No. 7,410,640, or US20050129701. In yet some other examples, an antigen binding domain against gp100 is an antigen binding portion, e.g., CDRs, of the antibody HMB45, NKIbetaB, or an antibody described in WO2013165940, or US20130295007. In yet some other examples, an antigen binding domain against tyrosinase is an antigen binding portion, e.g., CDRs, of an antibody described in, e.g., U.S. Pat. No. 5,843,674; or U.S. Ser. No. 19/950,504048. In yet some other examples, an antigen binding domain against EphA2 is an antigen binding portion, e.g., CDRs, of an antibody described in, e.g., Yu et al., Mol Ther 22(1):102-111 (2014). In yet some other examples, an antigen binding domain against GD3 is an antigen binding portion, e.g., CDRs, of an antibody described in, e.g., U.S. Pat. Nos. 7,253,263; 8,207,308; US 20120276046; EP1013761 A3; 20120276046; WO2005035577; or U.S. Pat. No. 6,437,098. In yet some other examples, an antigen binding domain against fucosyl GM1 is an antigen binding portion, e.g., CDRs, of an antibody described in, e.g., US20100297138; or WO2007/067992. In yet some other examples, an antigen binding domain against sLe is an antigen binding portion, e.g., CDRs, of the antibody G193 (for lewis Y), see Scott A M et al, Cancer Res 60: 3254-61 (2000), also as described in Neeson et al, J Immunol May 2013 190 (Meeting Abstract Supplement) 177.10. In yet some other examples, an antigen binding domain against GM3 is an antigen binding portion, e.g., CDRs, of the antibody CA 2523449 (mAb 14F7). In yet some other examples, an antigen binding domain against FIMWMAA is an antigen binding portion, e.g., CDRs, of an antibody described in, e.g., Kmiecik et al., Oncoimmunology 3(1):e27185 (2014) (PMID: 24575382) (mAb9.2.27); U.S. Pat. No. 6,528,481; WO2010033866; or US 20140004124. In yet some other examples, an antigen binding domain against o-acetyl-GD2 is an antigen binding portion, e.g., CDRs, of the antibody 8B6. In yet some other examples, an antigen binding domain against TEM1/CD248 is an antigen binding portion, e.g., CDRs, of an antibody described in, e.g., Marty et al., Cancer Lett 235(2):298-308 (2006); Zhao et al., J Immunol Methods 363(2):221-232 (2011). In yet some other examples, an antigen binding domain against CLDN6 is an antigen binding portion, e.g., CDRs, of the antibody IMABO27 (Ganymed Pharmaceuticals), see e.g., clinicaltrial.gov/show/NCT02054351. In yet some other examples, an antigen binding domain against TSHR is an antigen binding portion, e.g., CDRs, of an antibody described in, e.g., U.S. Pat. Nos. 8,603,466; 8,501,415; or U.S. Pat. No. 8,309,693. In yet some other examples, an antigen binding domain against GPRC5D is an antigen binding portion, e.g., CDRs, of the antibody FAB6300A (R&D Systems); or LS-A4180 (Lifespan Biosciences). In yet some other examples, an antigen binding domain against CD97 is an antigen binding portion, e.g., CDRs, of an antibody described in, e.g., U.S. Pat. No. 6,846,911; de Groot et al., J Immunol 183(6):4127-4134 (2009); or an antibody from R&D:MAB3734. In yet some other examples, an antigen binding domain against ALK is an antigen binding portion, e.g., CDRs, of an antibody described in, e.g., Mino-Kenudson et al., Clin Cancer Res 16(5):1561-1571 (2010). In yet some other examples, an antigen binding domain against polysialic acid is an antigen binding portion, e.g., CDRs, of an antibody described in, e.g., Nagae et al., J Biol Chem 288(47):33784-33796 (2013). In yet some other examples, an antigen binding domain against PLAC1 is an antigen binding portion, e.g., CDRs, of an antibody described in, e.g., Ghods et al., Biotechnol Appl Biochem 2013 doi:10.1002/bab.1177. In yet some other examples, an antigen binding domain against GloboH is an antigen binding portion of the antibody VK9; or an antibody described in, e.g., Kudryashov V et al, Glycoconj J.15(3):243-9 (1998), Lou et al., Proc Natl Acad Sci USA 111(7):2482-2487 (2014); MBrl: Bremer E-G et al. J Biol Chem 259:14773-14777 (1984). In yet some other examples, an antigen binding domain against NY—BR-1 is an antigen binding portion, e.g., CDRs of an antibody described in, e.g., Jager et al., Appl Immunohistochem Mol Morphol 15(1):77-83 (2007). In yet some other examples, an antigen binding domain against WT-1 is an antigen binding portion, e.g., CDRs, of an antibody described in, e.g., Dao et al., Sci Transl Med 5(176):176ra33 (2013); or WO2012/135854. In yet some other examples, an antigen binding domain against MAGE-A1 is an antigen binding portion, e.g., CDRs, of an antibody described in, e.g., Willemsen et al., J Immunol 174(12):7853-7858 (2005) (TCR-like scFv). In yet some other examples, an antigen binding domain against sperm protein 17 is an antigen binding portion, e.g., CDRs, of an antibody described in, e.g., Song et al., Target Oncol 2013 Aug. 14 (PMID: 23943313); Song et al., Med Oncol 29(4):2923-2931 (2012). In yet some other examples, an antigen binding domain against Tie 2 is an antigen binding portion, e.g., CDRs, of the antibody AB33 (Cell Signaling Technology). In one embodiment, an antigen binding domain against MAD-CT-2 is an antigen binding portion, e.g., CDRs, of an antibody described in, e.g., PMID: 2450952; U.S. Pat. No. 7,635,753. In yet some other examples, an antigen binding domain against Fos-related antigen 1 is an antigen binding portion, e.g., CDRs, of the antibody 12F9 (Novus Biologicals). In yet some other examples, an antigen binding domain against MelanA/MART1 is an antigen binding portion, e.g., CDRs, of an antibody described in, EP2514766 A2; or U.S. Pat. No. 7,749,719. In yet some other examples, an antigen binding domain against sarcoma translocation breakpoints is an antigen binding portion, e.g., CDRs, of an antibody described in, e.g., Luo et al, EMBO Mol. Med. 4(6):453-461 (2012). In yet some other examples, an antigen binding domain against TRP-2 is an antigen binding portion, e.g., CDRs, of an antibody described in, e.g., Wang et al, J Exp Med. 184(6):2207-16 (1996). In yet some other examples, an antigen binding domain against CYP1B1 is an antigen binding portion, e.g., CDRs, of an antibody described in, e.g., Maecker et al, Blood 102 (9): 3287-3294 (2003). In one embodiment, an antigen binding domain against RAGE-1 is an antigen binding portion, e.g., CDRs, of the antibody MAB5328 (EMD Millipore). In yet some other examples, an antigen binding domain against human telomerase reverse transcriptase is an antigen binding portion, e.g., CDRs, of the antibody cat no: LS-B95-100 (Lifespan Biosciences). In yet some other examples, an antigen binding domain against intestinal carboxyl esterase is an antigen binding portion, e.g., CDRs, of the antibody 4F12: cat no: LS-B6190-50 (Lifespan Biosciences). In yet some other examples, an antigen binding domain against mut hsp70-2 is an antigen binding portion, e.g., CDRs, of the antibody Lifespan Biosciences: monoclonal: cat no: LS-C133261-100 (Lifespan Biosciences). In yet some other examples, an antigen binding domain against CD79a is an antigen binding portion, e.g., CDRs, of the antibody Anti-CD79a antibody [HM47/A9] (ab3121), available from Abcam; antibody CD79A Antibody #3351 available from Cell Signalling Technology; or antibody HPA017748-Anti-CD79A antibody produced in rabbit, available from Sigma Aldrich. In yet some other examples, an antigen binding domain against CD79b is an antigen binding portion, e.g., CDRs, of the antibody polatuzumab vedotin, anti-CD79b described in Doman et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56th ASH Annual Meeting and Exposition, San Francisco, Calif. December 6-9, 2014. In yet some other examples, an antigen binding domain against CD72 is an antigen binding portion, e.g., CDRs, of the antibody J3-109 described in Leuk Lymphoma. 1995 June; 18(1-2):119-22; Cancer Res Mar. 15, 2009 69; 2358. In yet some other examples, an antigen binding domain against LAIR1 is an antigen binding portion, e.g., CDRs, of the antibody ANT-301 LAIR1 antibody, available from ProSpec; or anti-human CD305 (LAIR1) Antibody, available from BioLegend.

In yet some other examples, an antigen binding domain against FCAR is an antigen binding portion, e.g., CDRs, of the antibody CD89/FCAR Antibody (Catalog #10414-H08H), available from Sino Biological Inc. In yet some other examples, an antigen binding domain against LILRA2 is an antigen binding portion, e.g., CDRs, of the antibody LILRA2 monoclonal antibody (M17), clone 3C7, available from Abnova, or Mouse Anti-LILRA2 antibody, Monoclonal (2D7), available from Lifespan Biosciences. In yet some other examples, an antigen binding domain against CD300LF is an antigen binding portion, e.g., CDRs, of the antibody Mouse Anti-CMRF35-like molecule 1 antibody, Monoclonal[UP-D2], available from BioLegend, or Rat Anti-CMRF35-like molecule 1 antibody, Monoclonal[234903], available from R&D Systems. In yet some other examples, an antigen binding domain against CLEC12A is an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1.times.CD3 BiTE Antibody” 53^{rd} ASH Annual Meeting and Exposition, Dec. 10-13, 2011, and MCLA-117 (Merus). In yet some other examples, an antigen binding domain against BST2 (also called CD317) is an antigen binding portion, e.g., CDRs, of the antibody Mouse Anti-CD317 antibody, Monoclonal[3114], available from Antibodies-Online or Mouse Anti-CD317 antibody, Monoclonal[696739], available from R&D Systems. In yet some other examples, an antigen binding domain against EMR2 (also called CD312) is an antigen binding portion, e.g., CDRs, of the antibody Mouse Anti-CD312 antibody, Monoclonal[LS-B8033] available from Lifespan Biosciences, or Mouse Anti-CD312 antibody, Monoclonal[494025] available from R&D Systems. In yet some other examples, an antigen binding domain against LY75 is an antigen binding portion, e.g., CDRs, of the antibody Mouse Anti-Lymphocyte antigen 75 antibody, Monoclonal[HD30] available from EMD Millipore or Mouse Anti-Lymphocyte antigen 75 antibody, Monoclonal[A15797] available from Life Technologies. In still yet some other examples, an antigen binding domain against GPC3 is an antigen binding portion, e.g., CDRs, of the antibody hGC33 described in Anticancer Drugs. 2010 November; 21(10):907-916, or MDX-1414, HN3, or YP7, all three of which are described in FEBS Lett. 2014 Jan. 21; 588(2):377-82. In still yet some other examples, an antigen binding domain against FCRL5 is an antigen binding portion, e.g., CDRs, of the anti-FcRL5 antibody described in Mol Cancer Ther. 2012 October; 11(10):2222-32. In still yet some other examples, an antigen binding domain against IGLL1 is an antigen binding portion, e.g., CDRs, of the antibody Mouse Anti-Immunoglobulin lambda-like polypeptide 1 antibody, Monoclonal[AT1G4] available from Lifespan Biosciences, Mouse Anti-Immunoglobulin lambda-like polypeptide 1 antibody, Monoclonal[HSL11] available from BioLegendSad.

In still yet some other examples, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed above.

The antigen binding domain can be any domain that binds to the antigen including but not limited to a monoclonal antibody, a polyclonal antibody, a recombinant antibody, a human antibody, a humanized antibody, and a functional fragment thereof, including a Fab, a Fab′, a F(ab′)₂, an Fv, a single chain antibody (e.g., scFv), a minibody, a diabody, a single-domain antibody (“sdAb” or “nanobodies” or “camelids”), or an Fc binding domain.

In some instances, it may be beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some instances, the antigen binding domain are “cross-species” in that it binds to the counterpart antigen in a non-human primate, such as Callithrix jacchus, Saguinus oedipus or Saimiri sciureus, in order to facilitate a testing of immunogenicity of the antigen binding domain in these animals.

### Cytoplastic Domain of TFP or CAR

The cytoplasmic domain of the TFP or CAR can include an intracellular signaling domain. An intracellular signaling domain is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in some cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the TFP or CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of CD3 zeta, common FcR gamma (FCERIG), Fc gamma RIIa, FcR beta (Fc Epsilon R^{1b}), CD3 gamma, CD3 delta, CD3 epsilon, CD79a, CD79b, DAP10, and DAP12. In one embodiment, a CAR of the invention comprises an intracellular signaling domain, e.g., a primary signaling domain of CD3-zeta.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

The intracellular signaling domain of the TFP or CAR can comprise the CD3-zeta signaling domain by itself or it can be combined with any other desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

The intracellular signaling sequences within the cytoplasmic portion of the TFP or CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB.

### Transmembrane Domain of TFP or CAR

The extracellular region of TFP or CAR comprising an antigen binding domain can be linked to the intracellular region, for example by a transmembrane domain. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 or up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 or up to 15 amino acids of the intracellular region). In one aspect, the transmembrane domain is one that is associated with one of the other domains of the TFP or CAR is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another TFP on the TFP-T-cell surface (or another CAR on the CAR-T cell surface). In a different aspect the amino acid sequence of the transmembrane domain may be modified or substituted so as to minimize interactions with the binding domains of the native binding partner present in the same TFP or CAR.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the TFP or CAR has bound to a target. A transmembrane domain of particular use in this invention may include at least the transmembrane region(s) of e.g., the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Where desired, a hinge sequence or linker can be utilized to connect the extracellular domain to the transmembrane domain. Nonlimiting examples of hinge sequences are hinge sequences derived from a human immunoglobulin (Ig) hinge, e.g., an IgG4 hinge, or a CD8a hinge. A variety of linkers, oligo- or polypeptide linker, are available in the art for linking various domains together. They may vary in length from about 2 to 50 amino acids, and vary in amino acid composition. A commonly utilized linker is one enriched in glycine, e.g., amino acid sequence of GGGGSGGGGS, or variations thereof.

In some embodiments, the TFP- or the CAR-expressing cell described herein can further comprise multiple types of TFPs or CARs capable of binding to different antigens, or different epitopes on the same antigen. For instance, a TFP- or CAR-expressing cell of the present disclosure can comprise a second TFP or CAR that includes a different antigen binding domain, e.g., to the same target (CD19 or BCMA) or a different target (e.g., CD123). In one embodiment, when the TFP-expressing cell comprises two or more different TFPs or CARs, the antigen binding domains of the different TFPs or CARs can be such that the antigen binding domains do not interact with one another. For example, a cell expressing a first and second TFP can have an antigen binding domain of the first TFP, e.g., as a fragment, e.g., a scFv, that does not form an association with the antigen binding domain of the second TFP, e.g., the antigen binding domain of the second TFP is a VHH. Similarly, a cell expressing a first and second CAR can have an antigen binding domain of the first CAR, e.g., as a fragment, e.g., a scFv, that does not form an association with the antigen binding domain of the second CAR, e.g., the antigen binding domain of the second CAR is a VHH.

In some other embodiments, the TFP- or CAR-expressing cell described herein can further express another agent, e.g., an agent which enhances the activity of a TFP- or CAR-expressing cell. For example, in one embodiment, the agent can be an agent which inhibits an inhibitory molecule. Inhibitory molecules, e.g., PD1, can, in some embodiments, decrease the ability of a TFP- or CAR-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, LAG3, CTLA4, CD160, BTLA, LAIR1, TIM3, 2B4, CD93, OX40, Siglec-15, and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T-cells and myeloid cells (Agata et al. 1996 Int. Immunol 8:765-75). Two ligands for PD1, PD-L1 and PD-L2 have been shown to downregulate T-cell activation upon binding to PD1 (Freeman et al. 2000 J Exp Med 192:1027-34; Latchman et al. 2001 Nat Immunol 2:261-8; Carter et al. 2002 Eur J Immunol 32:634-43). Immune suppression can be reversed by inhibiting the local interaction of PD1 with PD-L1.

In one embodiment, the agent comprises the extracellular domain (ECD) of an inhibitory molecule, e.g., Programmed Death 1 (PD1) can be fused to a transmembrane domain and optionally an intracellular signaling domain such as 41BB and CD3 zeta (also referred to herein as a PD1 TFP). In one embodiment, the PD1 TFP, when used in combinations with an anti-CD19 TFP described herein, improves the persistence of the T-cell. In one embodiment, the TFP or CAR is comprising the extracellular domain of PD 1. Alternatively, provided are TFPs or CARs containing an antibody or antibody fragment such as a scFv that specifically binds to the Programmed Death-Ligand 1 (PD-L1) or Programmed Death-Ligand 2 (PD-L2).

In some embodiments, the present invention provides a population or a mixture of population of TFP- or CAR-expressing cells, in which PTPN2 expression or activity is downregulated (e.g., inhibited). In some examples, the population of TFP-expressing T-cells comprises a mixture of cells expressing different TFPs. The population of TFP-T-cells can include a first cell expressing a TFP having an anti-CD19 or anti-BCMA binding domain described herein, and a second cell expressing a TFP having a different anti-CD19 or anti-BCMA binding domain, e.g., an anti-CD19 or anti-BCMA binding domain described herein that differs from the anti-CD19 binding domain in the TFP expressed by the first cell. As another example, the population of TFP-expressing cells can include a first cell expressing a TFP that includes an anti-CD19 or anti-BCMA binding domain, e.g., as described herein, and a second cell expressing a TFP that includes an antigen binding domain to a target other than CD19 or BCMA (e.g., another tumor-associated antigen). The same approach may apply to a mixture of CAR-expressing cells, individual cells may target the same or different antigens.

Encompassed herein are also additional TFP or CAR configurations known in the art, including Split CARs, RCARs, as well as other TFP and CAR combinations described in WO2016187349, U.S. Pat. No. 9,856,497, WO2017123556, all of which are incorporated herein by reference in their entirety.

Further contemplated are allogeneic CAR-expressing cells in which its expression or activity of PTPN2 is inhibited. For example, the cell can be an allogeneic T cell, e.g., an allogeneic T cell lacking expression of a functional T cell receptor (TCR) and/or human leukocyte antigen (HLA), e.g., HLA class I and/or HLA class II. In particular, a T cell lacking a functional TCR can be, e.g., engineered such that it does not express any functional TCR on its surface, or engineered such that it does not express one or more subunits that comprise a functional TCR, or engineered such that it produces very little functional TCR on its surface. Alternatively, the T cell can express a substantially impaired TCR, e.g., by expression of mutated or truncated forms of one or more of the subunits of the TCR The term “substantially impaired TCR” means that this TCR will not substantially elicit an adverse immune reaction in a host.

Allogeneic T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, a CRISPR system, transcription activator like effector nuclease (TALEN), or zinc finger endonuclease (ZFN).

In some embodiments, an allogeneic cell can be a cell which does not express or expresses at low levels an inhibitory molecule, e.g. by any method described herein. For example, the cell can be a cell that does not express or expresses at low levels an inhibitory molecule, e.g., that can decrease the ability of a TFP- or CAR-expressing cell to mount an immune effector response. Examples of inhibitory molecules include PD1, PD-L1, CTLA4, TIM3, CEACAM (e.g., CEACAM-1, CEACAM-3 and/or CEACAM-5), LAGS, VISTA, BTLA, TIGIT, LAIR1, CD160, 2B4 and TGF beta.

The nucleic acid sequences coding for a desired TFP or CAR can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned. Where desired, the TFP- and CAR-expressing cells of the present inventions are generated using lentiviral viral vectors.

Conventional viral and non-viral based gene transfer methods can be used to introduce TFP- or CAR-encoding sequences to a cell of interest, e.g., a lymphoid cell as disclosed herein. Such methods can be used to introduce the TFP- or CAR-encoding sequences to cells in culture, which in turn is administered into a subject. Non-viral vector delivery systems can include DNA plasmids, RNA (e.g. a transcript of a vector described herein), naked nucleic acid, and nucleic acid complexed with a delivery vehicle, such as a liposome. Viral vector delivery systems can include DNA and RNA viruses, which can have either episomal or integrated genomes after delivery to the cell.

Viral based systems can include retroviral, lentivirus, adenoviral, adeno-associated and herpes simplex virus vectors for gene transfer. Integration in the host genome can occur with the retrovirus, lentivirus, and adeno-associated virus gene transfer methods, which can result in long term expression of the inserted sequence. High transduction efficiencies can be observed in many different cell types and target tissues.

A subject lymphoid cell in which its PTPN2 expression and/or activity is downregulated (e.g., inhibited) finds an array of utility in treating a range of diseases associated with the antigen to which the TFP or CAR binds. For instance, PTPN2 downregulation (e.g., inhibition) enhances lymphoid cell expansion, effector function, and survival of human TFP- or CAR-expressing T cells in vitro, and human T cell persistence and antitumor activity in vivo.

In one aspect, the present disclosure provides a method of augmenting activity of an effector cell (e.g., T cells, NK cells, KHYG cells). The method typically comprising: contacting said effector cell with an effective amount of a PTPN2 inhibitor (e.g., compound described herein) such that PTPN2 expression and activity is downregulated (e.g., inhibited) in said effector cell. Augmentation of effector activity can be evidenced by the cytolytic activity against a target cell such as a tumor or cancer cell, or helper activity including the release of cytokines. Assessing augmented effector function can be carried out using any methods known in the art or disclosed here. In some instances, cytotoxicity of an effector cell expressing TFP or CAR as disclosed herein can be greater in response to a PTPN2- inhibitor (e.g., compound described herein) treatment as compared to a control lymphoid cell lacking such treatment. A TFP- or CAR-expressing effector cell treated with a PTPN2 inhibitor (e.g., compound described herein) can be about 5%, 10%, 15%, 20%, 25%, 30%, 35%, 40%, 45%, 50%, 55%, 60%, 65%, 70%, 75%, 80%, 85%, 90%, 95%, 100%, 500% or more cytotoxic against tumor or cancer cells as compared to an effector cell lacking the treatment. In some embodiments, a change in cytotoxicity can comprise comparing such activity before and after treating the effector cell with a PTPN2 inhibitor (e.g., compound described herein). In some other instances, release of cytotoxic cytokines of an effector cell expressing TFP or CAR as disclosed herein can be greater in response to treating with a PTPN2 inhibitor (e.g., compound described herein) as compared to a control lymphoid cell lacking such treatment. Exemplary cytokines include IFNγ, TNFα, CSF, TGFβ, IL-1, IL-2, IL-4, IL-5, IL-6, IL-13, IL-17, IL-21, IL-22, granzyme, and the like. A TFP- or CAR-expressing effector cell can generate about 1 fold, 2 fold, 3 fold, 4 fold, 5 fold, 6 fold, 7 fold, 8 fold, 9 fold, 10 fold, 50 fold, 100 fold or greater release of cytotoxic cytokines in response to a PTPN2 inhibitor (e.g., compound described herein) treatment as compared to a control lymphoid cell that is not being exposed to the PTPN2 inhibitor (e.g., compound described herein).

In another aspect, the present disclosure provides a method of treating cancer in a subject in need thereof, comprising: administering to the subject an effective amount of lymphoid cells, wherein an individual lymphoid cell comprises (a) a chimeric T-cell receptor sequence encoding a T-cell receptor fusion protein (TFP), and/or (b) a chimeric antigen receptor (CAR) sequence encoding a CAR, wherein each of TFP and CAR, when present, exhibits specific binding to an antigen, and wherein expression and/or function of PTPN2 in said cell is downregulated (e.g., inhibited). In some embodiments of the disclosure, downregulation of PTPN2 expression and/or activity can be effected by one or more types of PTPN2 inhibitor (e.g., compound described herein) disclosed herein. Where desired, downregulation of expression or activity of PTPN2 takes place transiently by contacting the cells with a small molecule PTPN2 inhibitor (e.g., compound described herein) or a nucleic acid based PTPN2 inhibitor (e.g., siRNA or shRNA) that asserts such downregulation transiently without being integrated into the cell's genome. Alternatively, PTPN2 downregulation can occur permanently by contacting the cell with a PTPN2 inhibitor that disrupts the expression of the PTPN2 gene permanently by cleaving such gene with a CRISPR-based PTPN2 inhibitor.

In some examples, the practice of the subject method involves downregulating PTPN2 expression and/or activity in the lymphoid cells, ex vivo, prior to administering an effective amount of PTPN2-treated lymphoid cells (e.g., effector cells) to the subject. The ex vivo inhibition can be carried out prior to, concurrent with, or after the introduction of the nucleic acid encoding the TFP or CAR into the lymphoid cell. Such ex vivo treatment may facilitate the expansion and proliferation of the effector cells to yield to a cell count reaching a desired effective amount to be administered to a subject. Such ex vivo treatment may also prolong the survival effector cell persistence and antitumor activity in vivo. For instances, an effector cell of the present invention when infused into a subject is capable of killing tumor or cancer cells in the subject. Unlike antibody therapies, TFP- modified or CAR-modified immune effector cells (e.g., T cells, NK cells, KHYG cells) are able to replicate in vivo resulting in long-term persistence that can lead to sustained tumor control. In various aspects, the immune effector cells (e.g., T cells, NK cells, KHYG cells) administered to the subject, or their progeny, persist in the subject for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell or NK cell or KHYG cells to the subject.

Accordingly, the present disclosure also provides a method of increasing the therapeutic efficacy of a TFP- or CAR-expressing cell directed to a tumor or tumor associated antigen. In some embodiments, administering a PTPN2 inhibitor (e.g., compound described herein) occurs ex vivo. In other embodiments, administering a PTPN2 inhibitor (e.g., compound described herein) occurs in vivo prior to, concurrent with, or after the cells have been administered to a subject, where the cell may have or may not have previously been exposed to the PTPN2 inhibitor (e.g., compound described herein) ex vivo.

In one aspect, a fully-human TFP- or CAR-modified immune effector cells (e.g., T cells, NK cells, KHGY cells) of the invention may be a type of vaccine for ex vivo immunization and/or in vivo therapy in a mammal including a human.

The subject methods utilizing a TFP- or CAR-expressing lymphoid cells (including e.g., effector cells) that target one or more tumor antigens can be applied to treat solid tumor and hematological cancers. For example, the subject methods can be used to treat: Acute Lymphoblastic Leukemia (ALL), Acute Myeloid Leukemia (AML), Adrenocortical Carcinoma, Childhood Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Childhood (Brain Cancer), Atypical Teratoid/Rhabdoid Tumor, Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma—see Non-Hodgkin Lymphoma, Carcinoid Tumor (Gastrointestinal), Childhood Carcinoid Tumors, Cardiac (Heart) Tumors, Atypical Teratoid/Rhabdoid Tumor, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma (Mycosis Fungoides and S6zary Syndrome), Ductal Carcinoma In Situ (DCIS), Embryonal Tumors, Endometrial Cancer (Uterine Cancer), Ependymoma, Esophageal Cancer, Esthesioneuroblastoma(Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST), Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Langerhans Cell Hodgkin Lymphoma, Hypopharyngeal Cancer (Head and Neck Cancer), Islet Cell Tumors, Pancreatic Neuroendocrine Tumors, Kaposi Sarcoma(Soft Tissue Sarcoma), Kidney (Renal Cell) Cancer, Laryngeal Cancer (Head and Neck Cancer), Leukemia, Lip and Oral Cavity Cancer(Head and Neck Cancer), Liver Cancer, Lung Cancer (e.g., Non-Small Cell and Small Cell), Lymphoma, Male Breast Cancer, Malignant Fibrous Histiocytoma of Bone and Osteosarcoma, Melanoma, Merkel Cell Carcinoma(Skin Cancer), Mesothelioma, Malignant, Metastatic Cancer, Metastatic Squamous Neck Cancer with Occult Primary (Head and Neck Cancer), Midline Tract Carcinoma, Mouth Cancer(Head and Neck Cancer), Multiple Endocrine Neoplasia, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, CML, Myeloid Leukemia, Acute (AML), Myeloproliferative Neoplasms, Chronic, Nasal Cavity and Paranasal Sinus Cancer(Head and Neck Cancer), Nasopharyngeal Cancer(Head and Neck Cancer), Neuroblastoma, Non-Hodgkin Lymphoma, Non-Small Cell Lung Cancer, Oral Cancer, Lip and Oral Cavity Cancer and Oropharyngeal Cancer(Head and Neck Cancer), Osteosarcoma and Malignant Fibrous Histiocytoma of Bone, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Rectal Cancer, Retinoblastoma, Rhabdomyosarcoma, Salivary Gland Cancer (Head and Neck Cancer), Sarcoma, Childhood Rhabdomyosarcoma(Soft Tissue Sarcoma), Childhood Vascular Tumors (Soft Tissue Sarcoma), Ewing Sarcoma(Bone Cancer), Kaposi Sarcoma (Soft Tissue Sarcoma), Osteosarcoma(Bone Cancer), Soft Tissue Sarcoma, Uterine Sarcoma, S6zary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, T-Cell Lymphoma, Cutaneous, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter (Kidney (Renal Cell) Cancer), Ureter and Renal Pelvis, Transitional Cell Cancer (Kidney (Renal Cell) Cancer, Urethral Cancer, Uterine Cancer, Endometrial, Uterine Sarcoma, Vaginal Cancer, Vascular Tumors (Soft Tissue Sarcoma), Vulvar Cancer, and Wilms Tumor and Other Childhood Kidney Tumors, and any of the aforementioned cancers exhibiting expression and/or activity of PTPN2 in the cancer cells.

The present disclosure also provides a pharmaceutical compositions comprising a TFP- or CAR-expressing cell, e.g., a plurality of TFP-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity of administration will be determined by such factors as the condition of the subject, and the type and severity of the subject's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenesfaecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

The precise effective amount of the compositions of the present invention to be administered can be determined by a physician with consideration of individual differences in age, weight, tumor size, extent of infection or metastasis, and condition of the subject. It can generally be stated that a pharmaceutical composition comprising the T-cells described herein may be administered at a dosage of 10⁴ to 10⁹ cells/kg body weight, in some instances 10⁵ to 10⁶ cells/kg body weight, including all integer values within those ranges. T-cell compositions may also be administered multiple times at these dosages. The cells can be administered by using infusion techniques that are commonly known in immunotherapy (see, e.g., Rosenberg et al., New Eng. J. of Med. 319:1676, 1988).

In some examples, it may be desired to administer activated T-cells to a subject and then subsequently redraw blood (or have an apheresis performed), activate T-cells therefrom according to the present invention, and reinfuse the patient with these activated and expanded T-cells. This process can be carried out multiple times every few weeks. In certain aspects, T-cells can be activated from blood draws of from 10 cc to 400 cc. In certain aspects, T-cells are activated from blood draws of 20 cc, 30 cc, 40 cc, 50 cc, 60 cc, 70 cc, 80 cc, 90 cc, or 100 cc. In some embodiments, the T-cell compositions of the present invention are administered by i.v. injection. The compositions of T-cells may be injected directly into a tumor, lymph node, or site of infection.

In some examples, a subject may undergo leukapheresis, wherein leukocytes are collected, enriched, or depleted ex vivo to select and/or isolate the cells of interest, e.g., T-cells. These T-cell isolates may be expanded by methods known in the art and treated such that one or more TFP constructs of the invention may be introduced, thereby creating a TFP-expressing or CAR-expressing T-cell of the invention.

The following examples are given for the purpose of illustrating various embodiments of the disclosure and are not meant to limit the present disclosure in any fashion. The present examples, along with the methods and compositions described herein, are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### EXAMPLES

The following examples are provided for illustrative purposes only and not to limit the scope of the claims provided herein.

As used herein, the following abbreviations, unless otherwise indicated, shall be understood to have the following meanings:

| | | | |
|---|---|---|---|
| acetonitrile (ACN or | Diisopropylethylamine (DIPEA or | ethanol (EtOH) | mass spectroscopy |
| MeCN) | DIEA) | | (MS) |
| acetic acid (AcOH) | 4-(N,N-dimethylamino)pyridine | ethyl acetate (EtOAc) | Mesyl (Ms) |
| | (DMAP) | | |
| acetyl (Ac) | 1,2-dimethoxyethane (DME) | high performance liquid | nuclear magnetic |
| | | chromatography (HPLC) | resonance (NMR) |
| 2,2′- | N,N-dimethylformamide (DMF) | potassium | Phenyl (Ph) |
| bis(diphenylphosphino)- | | bis(trimethylsilyl)amide | |
| 1,1′-binaphthalene | | (KHMDS) | |
| (BINAP) | | | |
| Benzyl (Bn) | N,N-dimethylacetamide (DMA) | sodium | iso-propyl (iPr/i- |
| | | bis(trimethylsilyl)amide | Pr) |
| | | (NaHMDS) | |
| tert-butyl carbamate | Dimethylsulfoxide (DMSO) | lithium | reverse-phase |
| (BOC or Boc) | | bis(trimethylsilyl)amide | high-pressure |
| | | (LiHMDS) | liquid |
| | | | chromatography |
| | | | (RP-HPLC) |
| iso-butyl (i-Bu) | 1,1′-bis(diphenylphosphino)ferrocene | lithium aluminum | room temperature |
| | (Dppf or dppf) | anhydride (LAH) | (rt) |
| tert-butyl (t-Bu) | N-(3-dimethylaminopropyl)-N′- | liquid chromatography | tert- |
| | ethylcarbodiimide hydrochloride | mass spectrometry | butyldimethylsilyl |
| | (EDC or EDCI) | (LCMS) | (TBS) |
| dichloromethane | equivalent(s) (eq) | methyl (Me) | Triethylamine |
| (CH₂Cl₂) (DCM) | | | (TEA) |
| diisobutylaluminum | ethyl (Et) | methanol (MeOH) | trifluoroacetic acid |
| hydride (DIBAL-H) | | | (TFA) |
| | | | Tetrahydrofuran |
| | | | (THF) |

### Example 1: Compound Synthesis

### Example 1a: Synthesis of 5-(2-fluoro-6-methoxy-3-(1-methyl-1H-imidazol-4-yl)phenyl)-1,2,5-thiadiazolidin-3-one 1,1-dioxide (Compound 115)

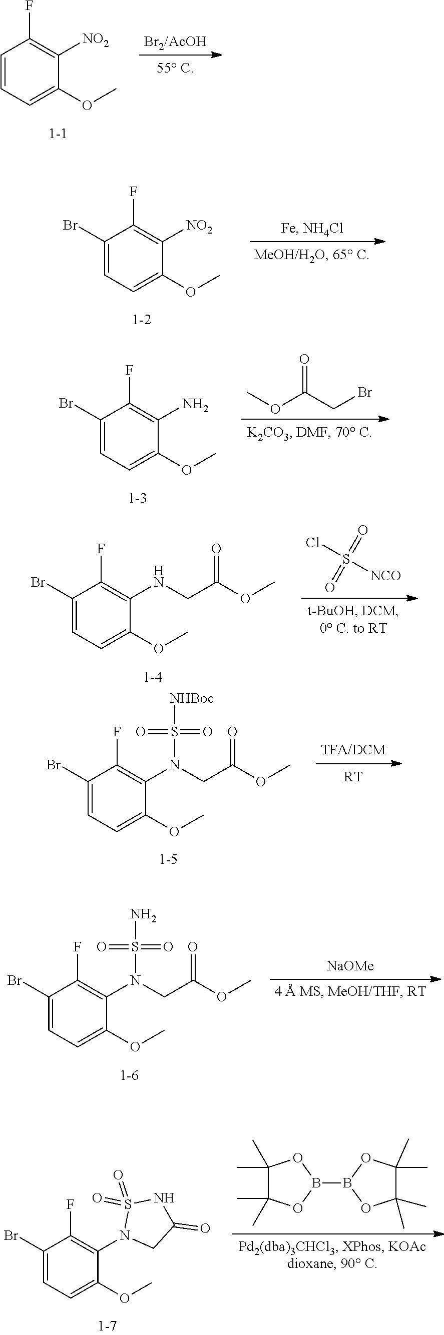

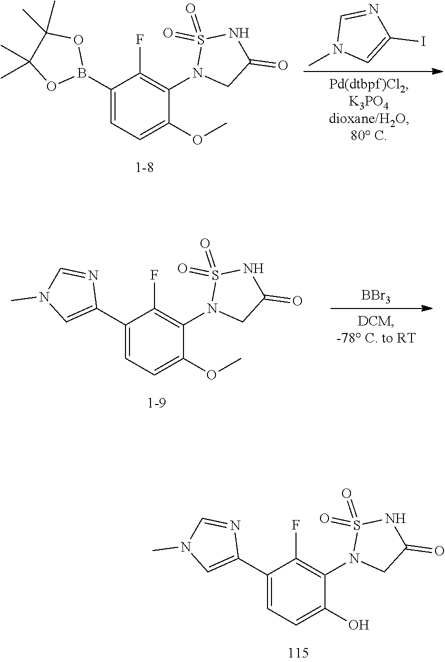

To a mixture of compound 1-1 (75 g, 438.6 mmol, 1.0 eq.) in AcOH (10 mL) being cooled to 0° C. was added Br₂ (90 mL, 1754.4 mmol, 4.0 eq.) dropwise. The reaction mixture was stirred at 55° C. for 15 h. The reaction mixture was concentrated under reduced pressure. The residue was diluted with water (2 L) and extracted with EtOAc (2 L×3). The organic layer was dried over anhydrous Na₂SO₄ and concentrated under reduced pressure to afford compound 1-2 (108 g). ¹H NMR (400 MHz, CDCl₃): δ 7.61 (dd, J=9.0, 7.7 Hz, 1H), 6.78 (dd, J=9.2, 1.3 Hz, 1H), 3.93 (s, 311).

To a mixture of compound 1-2 (108 g, 432.0 mmol, 1.0 eq.) and NH₄Cl (188 g, 3520 mmol, 8.0 eq.) in MeOH/water (400 mL/400 mL) was added Fe powder (98 g, 1760 mmol, 4.0 eq.), and the mixture was stirred at 65° C. for 3 h. The reaction mixture was cooled, poured into water (3 L) and extracted with petroleum ether/ethyl acetate (3/1, 2.5 L×3), dried over anhydrous Na₂SO₄, filtered and concentrated under reduced pressure to afford compound 1-3 (84 g). ¹H NMR (400 MHz, DMSO-d₆) δ 6.77 (t, J=8.1 Hz, 1H), 6.65 (d, J=8.8 Hz, 1H), 4.96 (s, 2H), 3.80 (s, 311).

To a mixture of compound 1-3 (20 g, 90.9 mmol, 1.0 eq.) and K₂CO₃ (50 g, 363.6 mmol, 4.0 eq.) in anhydrous DMF was added methyl 2-bromoacetate (20 g, 127.3 mmol, 1.4 eq.). The reaction mixture was stirred at 70° C. under Ar for 4 h. The reaction mixture was cooled, diluted with water (700 mL) and extracted with petroleum ether/ethyl acetate (3/1, 600 mL×3), dried over anhydrous Na₂SO₄, filtered and concentrated under reduced pressure. The residue was purified by column chromatography on silica gel eluting with petroleum ether/ethyl acetate (1/0-10/1) to afford compound 1-4 (13 g). LC-MS m/z: 294.1[M+H]⁺.

To a cooled (0° C.) mixture of sulfurisocyanatidic chloride (50.2 g, 356.0 mmol, 8.0 eq.) in anhydrous DCM (150 mL) was added t-BuOH (26.3 g, 356.0 mmol, 8.0 eq.) dropwise. The reaction mixture was stirred at 0° C. for 0.5 h. Then a solution of compound 1-4 (13 g, 44.5 mmol, 1.0 eq.) and TEA (72 g, 712.0 mmol, 16.0 eq.) in anhydrous DCM (150 mL) was added dropsies at 0° C. The resultant mixture was stirred at room temperature for 16 h. The reaction mixture was diluted with DCM (300 mL) and washed with H₂O (400 mL×2), dried over anhydrous Na₂SO₄, filtered, and concentrated under reduced pressure. The residue was purified by column chromatography on silica gel eluting with petroleum ether/ethyl acetate (1/0-1/1) to afford compound 1-5 (14.5 g). LC-MS m/z: 469.0 [M−H]⁻.

To a mixture of compound 1-5 (14.5 g, 30.8 mmol, 1.0 eq.) in anhydrous DCM (100 mL) was added TFA (10 mL). The reaction mixture was stirred at room temperature for 3 h. The reaction mixture was adjusted to pH=8-9 with aq. NaHCO₃ (sat.) and extracted with DCM (150 mL×3), dried over anhydrous Na₂SO₄, filtered, and concentrated under reduced pressure to afford compound 1-6 (9.0 g), which was used in the next step without further purification. LC-MS m/z: 368.9 [M−H]⁻.

To a mixture of compound 1-6 (9.0 g, 24.2 mmol, 1.0 eq.) and 4A molecular sieve (10 g) in anhydrous MeOH/THF (15 mL/90 mL) was added NaOMe (22 mL, 121 mmol, 5.4 M in MeOH, 5.0 eq.) under Ar. After addition, the mixture was stirred at room temperature under Ar for 2 h. The mixture was concentrated under reduced pressure. The residue was purified by column chromatography on silica gel with MeOH/EtOAc (0-15%) to afford compound 1-7 (7.2 g). LC-MS m/z: 338.8 [M−H]⁻.

To a mixture of compound 1-7 (2 g, 5.92 mmol, 1.0 eq.) and KOAc (1.74 g, 17.75 mmol, 3.0 eq.) in dioxane (30 mL) was added 4,4,4′,4′,5,5,5′,5′-octamethyl-2,2′-bi(1,3,2-dioxaborolane) (3.76 g, 14.8 mmol, 2.5eq.), X-phos (1.13 g, 2.37 mmol, 0.4 eq.) and Pd₂(dba)₃CH₃Cl (1.22 g, 1.18 mmol, 0.2 eq.). The reaction mixture was stirred at 90° C. for 16 h under N₂. The reaction mixture was concentrated under reduced pressure. The residue was purified by column chromatography on silica gel eluting with EtOAc/MeOH=1/0-4/1 to give compound 1-8 (1.5 g). LC-MS m/z: 385.1 [M−H]⁻.

To a mixture of compound 1-8 (100 mg, 0.259 mmol, 1.0 eq.), 4-iodo-1-methyl-1H-imidazole (81 mg, 0.389 mmol, 1.5 eq.) and K₃PO₄ (165 mg, 0.777 mmol, 3 eq.) in dioxane (6 mL) and H₂O (1.5 mL) was added Pd(dppf)Cl₂(17 mg, 0.026 mmol, 0.1 eq.). The reaction mixture was stirred at 80° C. under N₂ for 12 h. The reaction mixture was concentrated under reduced pressure. The residue was purified by column chromatography on silica gel eluting with methanol/ethyl acetate (0-50%) to obtain compound 1-9 (122 mg). LC-MS m/z: 338.9 [M−H]⁻.

To a cooled (−78° C.) solution of compound 1-9 (122 mg, 0.359 mmol, 1.0 eq.) in anhydrous DCM (2.5 mL) was added BBr₃ (2.5 mL) dropwise for 5 min. The reaction mixture was stirred at RT under N₂ for 2.5 h. The reaction mixture was quenched by MeOH (30 mL) and adjusted to pH=8-9 with NH₃—H₂O at −78° C. The reaction mixture was filtered, and the filtrate was concentrated to give crude product, which was purified by prep-HPLC (NH₃·H₂O) to obtain compound 115 (3.8 mg). LC-MS m/z: 325.0 [M−H]⁻; ¹H NMR (400 MHz, CD₃OD): δ 8.41 (s, 1H), 7.64-7.58 (m, 2H), 6.84 (d, J=7.6 Hz, 1H), 4.30 (s, 2H), 3.89 (s, 3H).

### Example 1b: Synthesis of 5-(3-(1-(4-chlorobenzyl)-1H-pyrazol-4-yl)-2-fluoro-6-hydroxyphenyl)-1,2,5-thiadiazolidin-3-one 1,1-dioxide (Compound 189)

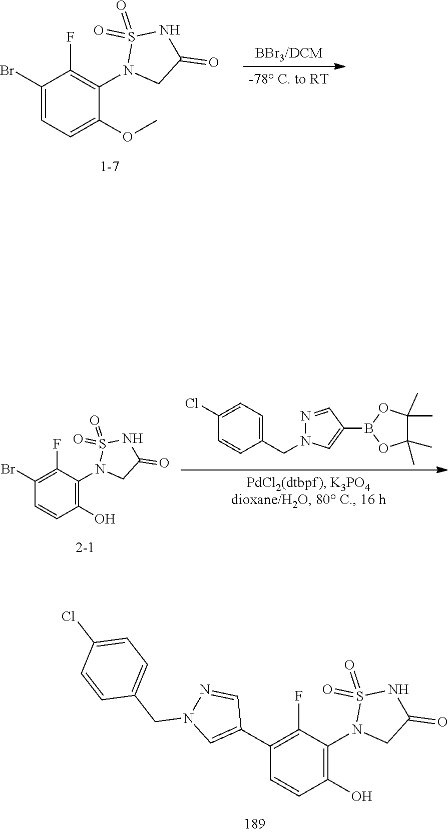 [SMILES: COc1ccc(Br)c(F)c1N1CC(=O)NS1(=O)=O ; "BBr3/DCM" ; O=C1CN(c2c(O)ccc(Br)c2F)S(=O)(=O)N1 ; CC1(C)OB(c2cnn(Cc3ccc(Cl)cc3)c2)OC1(C)C ; O=C1CN(c2c(O)ccc(-c3cnn(Cc4ccc(Cl)cc4)c3)c2F)S(=O)(=O)N1]

To a cooled (−78° C.) mixture of compound 1-7 (3.0 g, 8.85 mmol, 1.0 eq.) in anhydrous DCM (35 mL) was added BBr₃ (35 mL) dropwise. After addition, the mixture was stirred at room temperature for 4 h. The mixture was quenched with MeOH carefully and then adjusted pH=8-9 with NH₃·H₂O (28%, aq). The mixture was concentrated under reduced pressure. The residue was purified by column chromatography on silica gel with MeOH/EtOAc (0-15%) to afford compound 2-1 (2.0 g). LC-MS m/z: 324.9 [M−H]⁻.

To a mixture of compound 2-1 (80 mg, 0.246 mmol, 1.0 eq.), 1-(4-chlorobenzyl)-4-(4,4,5,5-tetramethyl-1,3,2-dioxaborolan-2-yl)-1H-pyrazole (118 mg, 0.369 mmol, 1.5 eq.) and K₃PO₄ (157 mg, 0.738 mmol, 3.0 eq.) in dioxane (2 mL) and H₂O (0.5 mL) was added PdCl₂(dtbpf) (24 mg, 0.0369 mmol, 0.15 eq.). The reaction mixture was stirred at 80° C. under Ar for 16 h. The reaction mixture was concentrated under reduced pressure. The residue was purified by column chromatography on silica gel eluting with methanol/ethyl acetate (0-100%) to give crude product, which was purified by prep-HPLC (NH₃·H₂O) to obtain compound 189 (22.9 mg). LC-MS m/z: 435.0 [M−H]⁻; ¹H NMR (400 MHz, CD₃OD): b δ8.04 (d, J=1.7 Hz, 1H), 7.85 (s, 1H), 7.47 (t, J=8.6 Hz, 1H), 7.40-7.31 (m, 2H), 7.24 (d, J=8.5 Hz, 2H), 6.76 (dd, J=8.7, 1.4 Hz, 1H), 5.36 (s, 2H), 4.30 (s, 2H).

### Example 1c: Synthesis of 5-(3-(2-aminobenzo[d]oxazol-6-yl)-2-fluoro-6-hydroxyphenyl)-1,2,5-thiadiazolidin-3-one 1,1-dioxide (Compound 191)

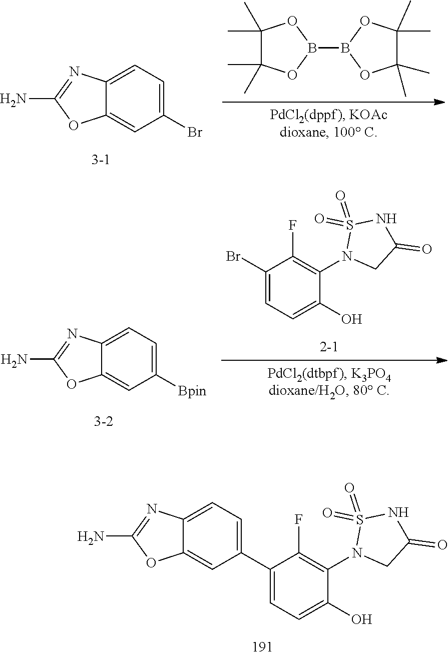

To a mixture of compound 3-1 (300 mg, 1.48 mmol, 1.0 eq.) and 4,4,4′,4′,5,5,5′,5′-octamethyl-2,2′-bi(1,3,2-dioxaborolane) (715 mg, 2.82 mmol, 2.0 eq.) in anhydrous dioxane (3 mL) were added KOAc (405 mg, 4.23 mmol, 3.0 eq.) and PdCl₂(dppf) (115 mg, 0.014 mmol, 0.1 eq.). The reaction mixture was stirred at 100° C. under Ar for 16 h. The reaction mixture was cooled, diluted with H₂O (30 mL) and extracted with EtOAc (20 mL×2). The organic layers were combined, dried over anhydrous Na₂SO₄, and concentrated under reduced pressure. The residue was purified by column chromatography on silica gel eluting with petroleum ether/ethyl acetate (1/0-5/1) to obtain compound 3-2 (163 mg). LC-MS m/z: 261.2 [M+H]⁺.

To a mixture of compound 3-2 (53 mg, 0.16 mmol, 1.0 eq.), compound 2-1 (63 mg, 0.24 mmol, 1.5 eq.) and K₃PO₄ (103 mg, 0.45 mmol, 3.0 eq.) in dioxane/H₂O (2 mL/0.5 mL) was added PdCl₂(dtbpf) (11 mg, 0.016 mmol, 0.1 eq.). The reaction mixture was stirred at 80° C. under Ar for 16 h. The mixture was concentrated under reduced pressure. The residue was purified by column chromatography on silica gel eluting with MeOH/ethyl acetate (0-20%) to give crude product, which was purified by pre-HPLC (NH₄HCO₃) to obtain compound 191 (8.9 mg). LC-MS m/z: 377.0 [M−H]⁻; ¹H NMR (400 MHz, CD₃OD): δ 7.44 (s, 1H), 7.34-7.23 (m, 311), 6.81 (d, J=8.7 Hz, 1H), 4.32 (s, 2H).

### Example 1d: Synthesis of 5-(2-chloro-6-hydroxy-3-(1-methyl-1H-pyrazol-4-yl)phenyl)-1,2,5-thiadiazolidin-3-one 1,1-dioxide (Compound 311)

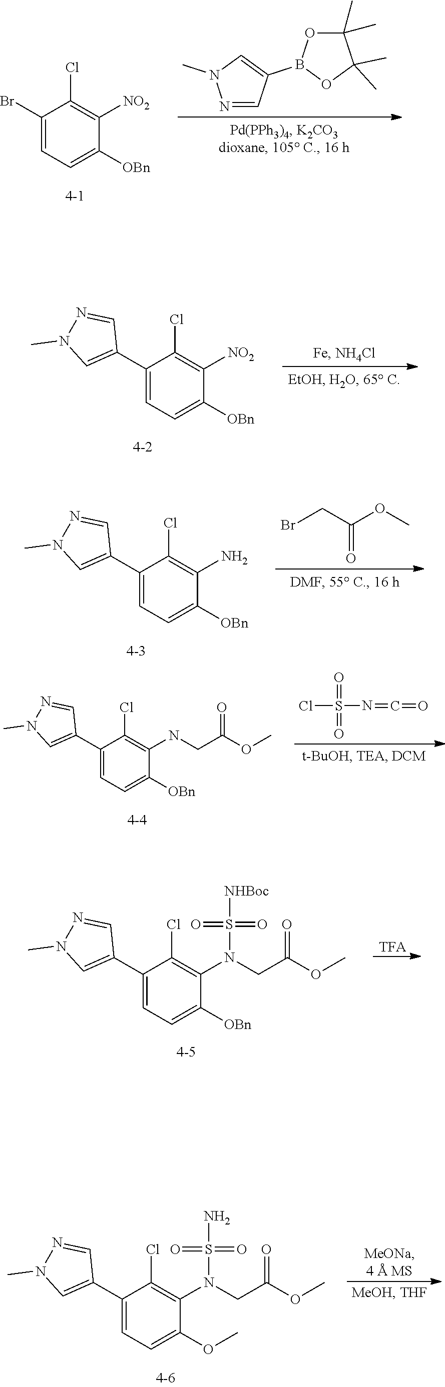

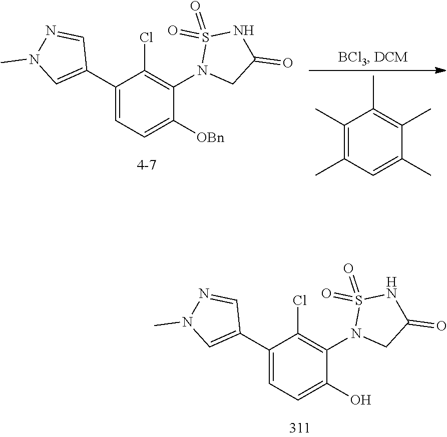

To a mixture of compound 4-1 (400 mg, 1.17 mmol, 1.0 eq) in dioxane (5 mL) were added 1-methyl-4-(4,4,5,5-tetramethyl-1,3,2-dioxaborolan-2-yl)-1H-pyrazole (293 mg, 1.41 mmol, 1.2 eq), H₂O (0.5 mL), K₂CO₃ (324 mg, 2.34 mmol, 2.0 eq) and Pd(PPh₃)₄ (136 mg, 0.12 mmol, 0.1 eq) under N₂. The reaction mixture was stirred at 105° C. under N₂ for 16 h. The reaction mixture was concentrated under reduced pressure. The residue was purified by column (PE/EA=2:1) to give a compound 4-2 (380 mg). ESI-MS m/z: 344.0 [M+H]⁺.

To a mixture of compound 4-2 (380 mg, 1.11 mmol, 1.0 eq) in EtOH (10 mL) were added NH₄Cl (475 mg, 8.88 mmol, 8.0 eq), Fe (248 mg, 4.44 mmol, 4.0 eq) and H₂O (2 mL). The mixture was stirred at 65° C. under N₂ for 1 h. The reaction mixture was filtered. The filtrate was diluted with H₂O (15 mL) and extracted with EA (3×15 mL). The combined organic layers were washed with brine, dried over Na₂SO₄, filtered, and concentrated under reduced pressure. The residue was purified by column (PE/EA=2:1) to give a compound 4-3 (175 mg). ESI-MS m/z: 314.0 [M+H]⁺.

To a mixture of compound 4-3 (110 mg, 0.351 mmol, 1.0 eq) in DMF (5 mL) were added methyl 2-bromoacetate (81 mg, 0.527 mmol, 1.5 eq) and K₂CO₃ (146 mg, 1.05 mmol, 3.0 eq) under N₂. The reaction mixture was stirred at 55° C. for 16 h. The reaction mixture was diluted with EA (20 mL) and washed with brine (3×20 mL). The organic layer was washed with brine, dried over Na₂SO₄, filtered, and concentrated under reduced pressure. The residue was purified by column chromatography on silica gel eluting with PE/EA=(3:1) to give a compound 4-4 (50 mg). ESI-MS m/z: 386.2 [M+H]⁺.

To mixture of sulfurisocyanatidic chloride (184 mg, 1.3 mmol, 10 eq) in DCM (5 mL) was added t-BuOH (96.2 mg, 1.3 mmol, 10 eq) at 0° C. under N₂. The reaction mixture was stirred at rt for 1 h. Then, compound 4-4 (50 mg, 0.13 mmol, 1.0 eq) and TEA (260.3 mg, 2.6 mmol, 20 eq) were added at 0° C. The reaction mixture was stirred at rt overnight under N₂. The mixture was quenched with water (20 mL) and extracted with DCM (30 mL). The organic layer was separated, dried over anhydrous Na₂SO₄, filtered, and concentrated under reduced pressure to afford compound 4-5 (150 mg crude), which was used in the next step without further purification. ESI-MS m/z: 565.2 [M+H]⁺.

To mixture of compound 4-5 (150 mg, 0.266 mmol, 1.0 eq) in DCM (5 mL) was added TFA (2 mL). The reaction mixture was stirred at rt for 2 h. The reaction mixture was adjusted to pH=8-9 with aq. NaHCO₃ solution and extracted with DCM (3×15 mL). The combined organic layers were washed with brine, dried over Na₂SO₄, filtered and concentrated under reduced pressure to give compound 4-6 (100 mg crude), which was used in the next step without further purification. ESI-MS m/z: 465.2 [M+H]⁺.

To mixture of compound 4-6 (100 mg, 0.215 mmol, 1.0 eq) in THF (2 mL) and MeOH (0.5 mL) were added 4A MS (100 mg) and MeONa (0.3 mL, 5.4 M) under N₂. The reaction was stirred at rt for 2 h. The reaction mixture concentrated under reduced pressure. The residue was purified by flash column chromatography (EA: MeOH=4:1) to afford compound 4-7 (50 mg). ESI-MS m/z: 431.0 [M−H]⁻.

To mixture of compound 4-7 (50 mg, 0.116 mmol, 1.0 eq) in DCM (2 mL) were added 1,2,3,4,5-pentamethylbenzene (51.47 mg, 0.347 mmol, 3.0 eq) and BCl₃ (0.5 mL) at −78° C. under N₂. The reaction was stirred at rt for 3 h. The mixture was quenched with MeOH (5 mL) at −78° C. and adjusted to pH=8-9 with NH₃·H₂O. The mixture was filtered, and the filtrate was purified by prep-HPLC to afford compound 311 (15 mg). ESI-MS m/z: [M−H]⁻; 1H NMR (400 MHz, CD₃OD): δ 7.90 (s, 1H), 7.70 (s, 1H), 7.34 (d, J=8.6 Hz, 1H), 6.90 (d, J=8.6 Hz, 1H), 4.31 (s, 2H), 3.93 (s, 311).

### Example 1e: Synthesis of 5-(2-fluoro-6-hydroxy-3-(1-isopropyl-2,5-dihydro-1H-pyrrol-3-yl)phenyl)-1,2,5-thiadiazolidin-3-one 1,1-dioxide (Compound 461)

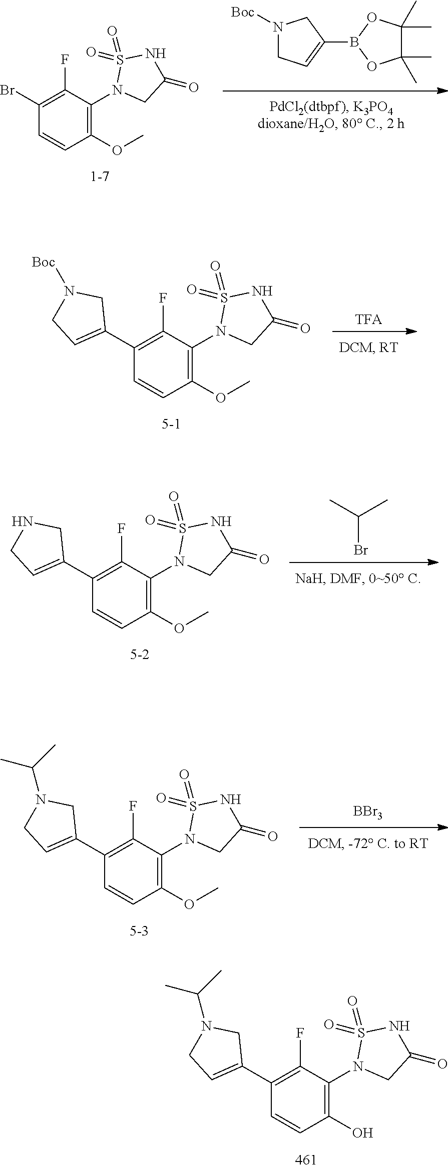

To a mixture of compound 1-7 (500 mg, 1.47 mmol, 1.0 eq), tert-butyl 3-(4,4,5,5-tetramethyl-1,3,2-dioxaborolan-2-yl)-2,5-dihydro-1H-pyrrole-1-carboxylate (522 mg, 1.77 mmol, 1.2 eq) and K₃PO₄ (782 mg, 3.68 mmol, 2.5 eq) in dioxane (16 mL) and H₂O (4 mL) was added PdCl₂(dtbpf) (96 mg, 0.15 mmol, 0.1 eq). The reaction mixture was stirred at 80° C. under Ar for 2 h. The reaction mixture was concentrated under reduced pressure. The residue was purified by column chromatography on silica gel eluting with methanol/ethyl acetate (0-100%) to give compound 5-1 (450 mg). ESI-MS m/z: 426.1 [M−H]⁻.

To a mixture of compound 5-1 (835 mg, 1.95 mmol, 1.0 eq) in DCM (9 mL) was added TFA (3 mL). The reaction mixture was stirred at RT for 2 h. The reaction mixture was neutralized with saturated NaHCO₃ solution, and then concentrated under reduced pressure. The residue was purified by reverse column chromatography (NH₄HCO₃) to give compound 5-2 (534 mg). ESI-MS m/z: 326.1 [M−H]⁻.

To a mixture of compound 5-2 (150 mg, 0.46 mmol, 1.0 eq) in dry DMF (2 mL) was added NaH (29 mg, 0.73 mmol, 1.6 eq) under Ar. The reaction mixture was stirred at 30° C. for 1 h. Then, a solution of 2-bromopropane in DMF (2 mL) was added at 0° C. and the resulting mixture was stirred at 50° C. overnight. The reaction was quenched with slow addition of MeOH, then concentrated under reduced pressure. The residue was purified by flash column chromatography on silica gel (eluted with methanol in ethyl acetate from 0% to 100%) to give compound 5-3 (45 mg). ESI-MS m/z: 368.1 [M−H]⁻.

To a mixture of compound 5-3 (45 g, 0.12 mmol, 1.0 eq) in anhydrous DCM (1 mL) at −72° C. was added BBr₃ (1 mL) under an argon atmosphere. After addition, the mixture was stirred at room temperature for 6 h. The mixture was quenched with MeOH carefully at −72° C. and then adjusted to pH=8-9 with NH₃·H₂O (28%, aq). The mixture was concentrated under reduced pressure. The residue was treated with MeOH and DMF and filtered to remove inorganic salt. The filtrate was concentrated under reduced pressure. The residue was purified by prep-HPLC to afford compound 461 (2.84 mg). ESI-MS m/z: 354.1 [M−H]⁻; ¹H NMR (400 MHz, CD₃OD): δ 7.31 (t, J=8.5 Hz, 1H), 6.79 (d, J=8.8 Hz, 1H), 6.28 (s, 1H), 4.43 (s, 2H), 4.25 (s, 2H), 4.19 (s, 2H), 3.68-3.58 (m, 1H), 1.38 (d, J=6.4 Hz, 6H).

### Example 1f: Synthesis of 5-(3-(1-(cyclohexylmethyl)-1H-imidazol-4-yl)-2-fluoro-6-hydroxyphenyl)isothiazol-3(2H)-one 1,1-dioxide (Compound 277)

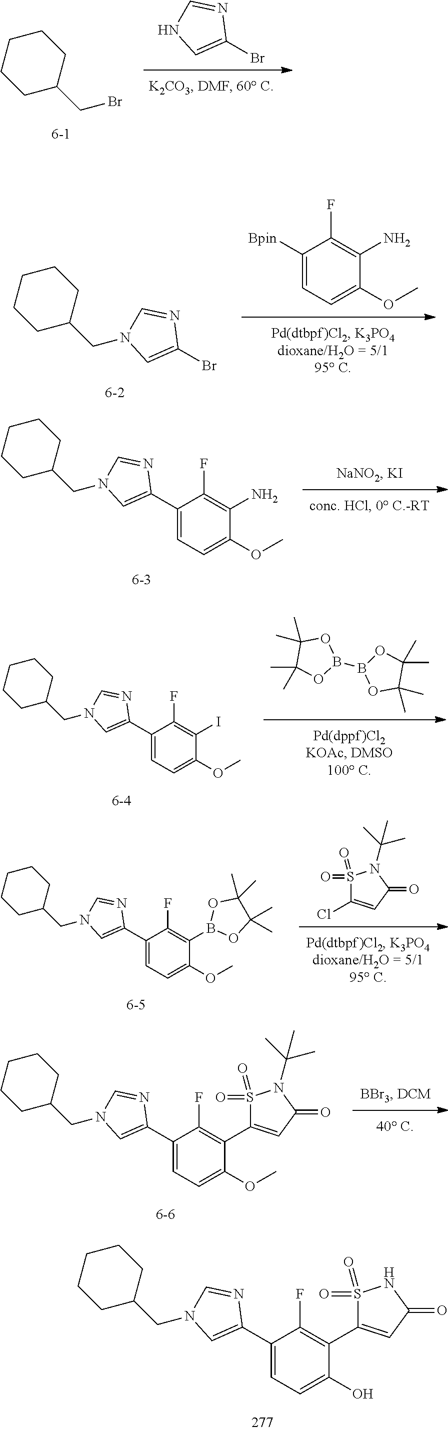

A mixture of compound 6-1 (11 g, 62.5 mmol, 1.0 eq), 4-bromo-1H-imidazole (9 g, 62.5 mmol, 1.0 eq) and K₂CO₃ (26 g, 187.5 mmol, 3 eq) in DMF (100 mL) was stirred at 60° C. overnight. This mixture was diluted with water (200 mL) and extracted with EtOAc (100 mL×3). The combined organic layers were washed with brine (300 mL), dried over anhydrous Na₂SO₄, and concentrated in vacuo. The residue was purified by column (EtOAc/PE=1/3) to afford compound 6-2 as a colorless oil (7.5 g). ESI-MS m/z: 245.0 [M+H]⁺.

A mixture of compound 6-2 (5 g, 20.66 mmol, 1.0 eq), 2-fluoro-6-methoxy-3-(4,4,5,5-tetramethyl-1,3,2-dioxaborolan-2-yl)aniline (5.5 g, 20.66 mmol, 1.0 eq), Pd(dtbpf)Cl₂ (1.35 g, 2.07 mmol, 0.1 eq) and K₃PO₄ (13.14 g, 61.98 mmol, 3 eq) in dioxane/H₂O=5/1 (72 mL) was stirred under N₂ at 95° C. overnight. The mixture was concentrated in vacuo and the residue was purified by column (EtOAc/PE=1/2) to afford compound 6-3 (2.6 g). ESI-MS m/z: 304.3 [M+H]⁺.

A solution of compound 6-3 (2.6 g, 8.6 mmol, 1.0 eq) in conc. HCl (60 mL) was stirred at 0° C. for 30 min. KI (7.12 g, 43 mmol, 5 eq) and NaNO₂ (1.78 g, 25.7 mmol, 3 eq) were added to the above solution and the mixture was stirred at RT for 1 h. The mixture was diluted with water (100 mL) and extracted with EtOAc (50 mL×3). The combined organic layer was dried over anhydrous Na₂SO₄, filtered and concentrated in vacuo. The residue was purified by column (EtOAc/PE=1/1) to afford compound 6-4 (2.5 g). ESI-MS m/z: 415.0 [M+H]⁺.

A solution of compound 6-4 (2.5 g, 6.4 mmol, 1.0 eq), 4,4,4′,4′,5,5,5′,5′-octamethyl-2,2′-bi(1,3,2-dioxaborolane) (3.1 g, 12.8 mmol, 2.0 eq), Pd(dppf)Cl₂(468 mg, 0.64 mmol, 0.1 eq) and KOAc (18.8 g, 19.2 mmol, 3 eq) in DMSO (30 mL) was stirred at 100° C. for 3 h. The mixture was diluted with water (60 mL) and extracted with EtOAc (30 mL×3). The combined organic layer was washed with brine (100 mL), dried over anhydrous Na₂SO₄, and concentrated in vacuo. The residue was purified by column (EtOAc/PE=1/1) to afford compound 6-5 (1.65 g). ESI-MS m/z: 415.0 [M+H]⁺.

A mixture of compound 6-5 (1.65 g, 3.98 mmol, 1.0 eq), 2-(tert-butyl)-5-chloroisothiazol-3(2H)-one 1,1-dioxide (710 mg, 3.18 mmol, 0.8 eq), Pd(dtbpf)Cl₂ (261 mg, 0.4 mmol, 0.1 eq) and K₃PO₄ (2.5 g, 11.94 mmol, 3 eq) in dioxane/H₂O=5/1 (25 mL) was stirred under N₂ at 95° C. overnight. The mixture was concentrated in vacuo and the residue was purified by column (EtOAc/PE=3/1) to afford compound 6-6 (770 mg). ESI-MS m/z: 476.2 [M−H]⁻.

A solution of compound 6-6 (770 mg, 1.62 mmol, 1.0 eq) in DCM (2 mL) was stirred at −60° C. and BBr₃ (10 mL) was added to the above solution. The mixture was stirred at RT for 2 h. The mixture was cooled to −60° C., quenched with methanol and adjusted to pH=8 with NH₃ in methanol. The mixture was filtered, and the filtrate was concentrated in vacuo. The residue was purified by Prep-HPLC to afford compound 277 (40.1 mg). ESI-MS m/z: 404.1 [M−H]⁻; ¹H NMR (400 MHz, CD₃OD): δ 7.86 (t, J=8.8 Hz, 1H), 7.73 (s, 1H), 7.43 (d, J=3.2 Hz, 1H), 6.82 (d, J=8.8 Hz, 1H), 6.76 (s, 1H), 3.90 (d, J=7.2 Hz, 2H), 1.80-1.72 (m, 311), 1.70-1.60 (m, 311), 1.32-1.20 (m, 3H), 1.07-0.97 (m, 2H).

### Example 1g: Synthesis of 5-(3-(1-(cyclohexylmethyl)-1H-imidazol-4-yl)-2-fluoro-6-hydroxyphenyl)isothiazolidin-3-one 1,1-dioxide (Compound 419)

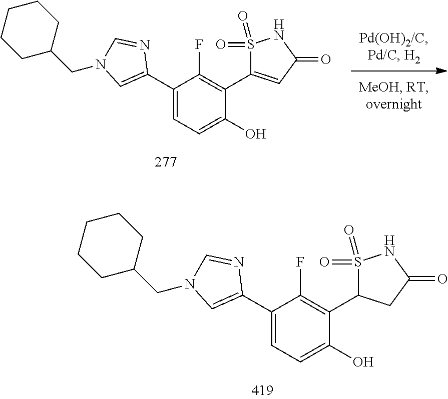 [SMILES: O=C1C=C(c2c(O)ccc(-c3cn(CC4CCCCC4)cn3)c2F)S(=O)(=O)N1 ; "Pd(OH)2/C,Pd/C, H2" ; O=C1CC(c2c(O)ccc(-c3cn(CC4CCCCC4)cn3)c2F)S(=O)(=O)N1]

A solution of compound 277 (800 mg), Pd/C (100 mg) and Pd (011)₂/C (100 mg) in MeOH (20 mL) was stirred under H₂ at RT overnight. The mixture was filtered, and the filtrate was concentrated in vacuo. The residue was purified by prep-HPLC to afford compound 419 (27.97 mg). ESI-MS m/z: 406.3 [M−H]⁻; ¹H NMR (400 MHz, CD₃OD): δ 8.35 (s, 1H), 7.64-7.56 (m, 2H), 6.78 (d, J=8.8 Hz, 1H), 5.38-5.31 (m, 1H), 3.98 (d, J=7.2 Hz, 2H), 1.91-1.80 (m, 1H), 1.80-1.61 (m, 6H), 1.38-1.16 (m, 4H), 1.07- 0.97 (m, 2H).

### Example 1h: Synthesis of 5-(3-(5-cyclopropyl-1-(cyclopropylmethyl)-1H-pyrazol-3-yl)-2-fluoro-6-hydroxyphenyl)-1,2,5-thiadiazolidin-3-one 1,1-dioxide (Compound 409)

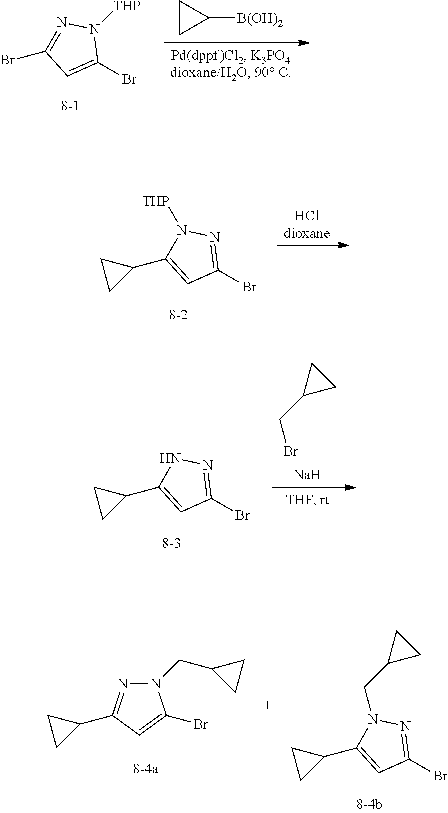

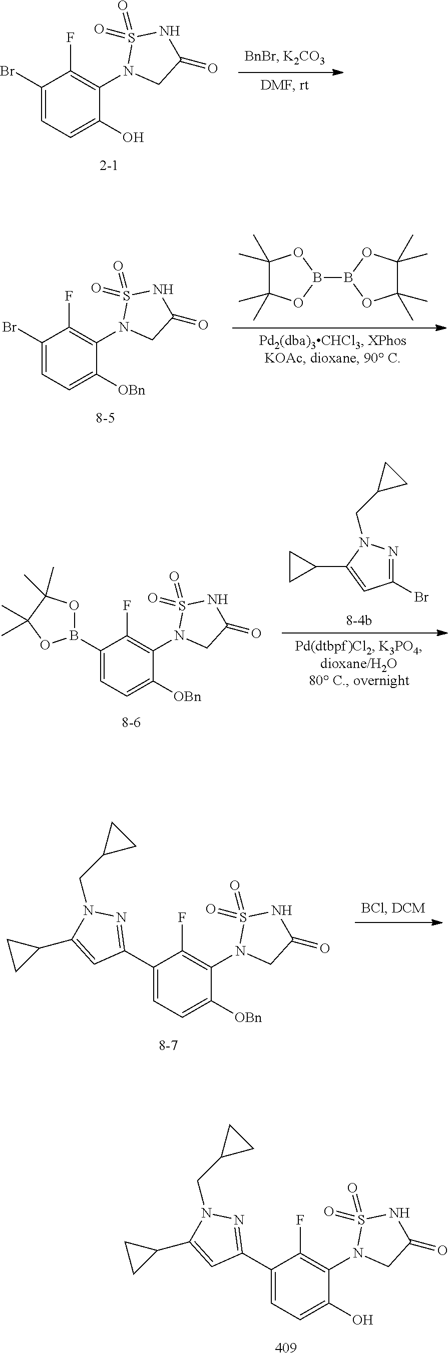

A mixture of compound 8-1 (2.00 g, 6.47 mmol, 1.0 eq), cyclopropyl boronic acid (631 mg, 7.34 mmol, 1.1 eq), Pd(dppf)Cl₂ (250 mg, 0.34 mmol, 0.05 eq) and K₃PO₄ (4.12 g, 19.42 mmol, 3.0 eq) in dioxane/H₂O (7.5 mL/1.5 mL) was stirred at 90° C. overnight. The mixture was washed with water (20 mL) and extracted with EtOAc (20 mL×3). The organic layer was dried over Na₂SO₄, filtered, and concentrated in vacuo. The residue was purified by column (EtOAc/PE=0-10%) to afford compound 8-2 (900 mg).

To a mixture of compound 8-2 (900 mg, 3.33 mmol, 1.0 eq) in DCM (5.0 mL) was added HCl/dioxane (5.0 mL, 4 M) at room temperature. Then the reaction mixture was stirred at rt for 4 h. The reaction mixture was washed with water (20 mL) and extracted with EtOAc (20 mL×3). The organic layer was dried over Na₂SO₄, filtered and concentrated in vacuo to afford compound 8-3 (230 mg, crude). ESI-MS m/z: 187.2 [M+H]⁺.

To a mixture of compound 8-3 (230 mg, 1.24 mmol, 1.0 eq) in THF (10 mL) at 0° C. under N₂ atmosphere was added NaH (60 mg, 1.96 mmol, 1.5 eq). After stirring for 30 min, (bromomethyl)cyclopropane (335 mg, 2.48 mmol, 2.0 eq) was added at 0° C. and the mixture was stirred at rt for 16 h. This mixture was diluted with water (20 mL) and extracted with EtOAc (15 mL×3), and the organic layer was combined. The organic layer was washed with saturated brine (30 mL), dried over anhydrous Na₂SO₄ and concentrated in vacuo. The crude was purified by column chromatography on silica gel eluting with (PE/EA=5/1) to afford compound 8-4a as colorless oil (90 mg) and compound 8-4b (70 mg). For 8-4a: 1H NMR (400 MHz, CDCl₃): δ 5.91 (s, 11), 3.95 (d, J=6.8 Hz, 2H), 1.89 (td, J=8.6, 4.6 Hz, 1H), 1.34-1.25 (m, 1H), 0.94-0.85 (m, 2H), 0.72-0.63 (m, 2H), 0.54 (q, J=5.4 Hz, 2H), 0.40 (q, J=5.2 Hz, 2H). For 8-4b: 1H NMR (400 MHz, CDCl₃) δ 5.82 (s, 1H), 4.01 (d, J=6.8 Hz, 2H), 1.78-1.67 (m, 1H), 1.35-1.24 (m, 1H), 1.01-0.94 (m, 2H), 0.70-0.64 (m, 2H), 0.61-0.54 (m, 2H), 0.38 (q, J=4.8 Hz, 2H).

To a mixture of compound 2-1 (20.7 g, 63.69 mmol, 1.0 eq), K₂CO₃ (13.2 g, 95.54 mmol, 1.5 eq) and anhydrous DMF (200 mL) was added BnBr (10.89 g, 63.69 mmol, 1.0 eq) under Ar. After addition, the mixture was stirred at room temperature under Ar for 16 h. The mixture was concentrated under reduced pressure. The residue was purified by column chromatography on silica gel with MeOH/EtOAc (0-15%) to afford compound 8-5 (11.3 g). ESI-MS m/z: 412.9 [M−H]⁻.

To a mixture of compound 8-5 (11.3 g, 27.23 mmol, 1.0 eq), 4,4,4′,4′,5,5,5′,5′-octamethyl-2,2′-bi(1,3,2-dioxaborolane) (17.28 g, 68.07 mmol, 2.5 eq), Xphos (5.19 g, 10.89 mmol, 0.4 eq) and KOAc (8.02 g, 81.69 mmol, 3.0 eq) in anhydrous dioxane (200 mL) was added Pd₂(dba)₃-CHCl₃ (5.64 g, 5.45 mmol, 0.2 eq) under Ar. After addition, the mixture was stirred at 90° C. under Ar for 16 h. The mixture was concentrated under reduced pressure. The residue was purified by column chromatography on silica gel with MeOH/EtOAc (0-15%) to afford compound 8-6 (9.5 g). ESI-MS m/z: 461.2 [M−H]⁻.

A mixture of compound 8-4b (70 mg, 0.29 mmol, 1.5 eq), compound 8-6 (80 mg, 0.17 mmol, 1.0 eq), Pd(dtbpf)Cl₂(13 mg, 0.03 mmol, 0.1 eq) and K₃PO₄ (110 mg, 0.51 mmol, 3 eq) in dioxane/H₂O=5/1 (2.5 mL) was protected by nitrogen and stirred at 90° C. overnight. The mixture was concentrated in vacuo. The residue was purified by column (DCM/MeOH=3/1) to afford compound 8-7 (70 mg). ESI-MS m/z: 495.2 [M−H]⁻.

To a mixture of compound 8-7 (70 mg, 0.14 mmol, 1.0 eq) and Me₅Ph (63 mg, 0.43 mmol, 3.0 eq) in DCM (2 mL) was added BCl₃ (2 mL) at −78° C. After addition, the mixture was stirred at 30° C. for 2 h under N₂ atmosphere. The mixture was concentrated and quenched by NH₃-MeOH dropwise at −78° C. to pH-8. The reaction mixture was filtered and concentrated to give crude product, which was purified by prep-HPLC (CAN/H₂O/NH₃·H₂O) to obtain compound 409 (9.2 mg). ESI-MS m/z: 405.0 [M−H]⁻; ¹H NMR (400 MHz, CD₃OD): δ 7.67 (t, J=8.4 Hz, 1H), 6.79 (d, J=8.6 Hz, 1H), 6.20 (d, J=3.4 Hz, 1H), 4.34 (s, 2H), 4.14 (d, J=6.8 Hz, 2H), 1.98-1.82 (m, 1H), 1.44-1.28 (m, 1H), 1.12-0.98 (m, 2H), 0.84-0.70 (m, 2H), 0.64-0.56 (m, 2H), 0.52-0.40 (m, 2H).

### Example 1i: Synthesis of 5-(3-(1-(cyclopropylsulfonyl)-2,5-dihydro-1H-pyrrol-3-yl)-2-fluoro-6-hydroxyphenyl)-1,2,5-thiadiazolidin-3-one 1,1-dioxide (Compound 238)

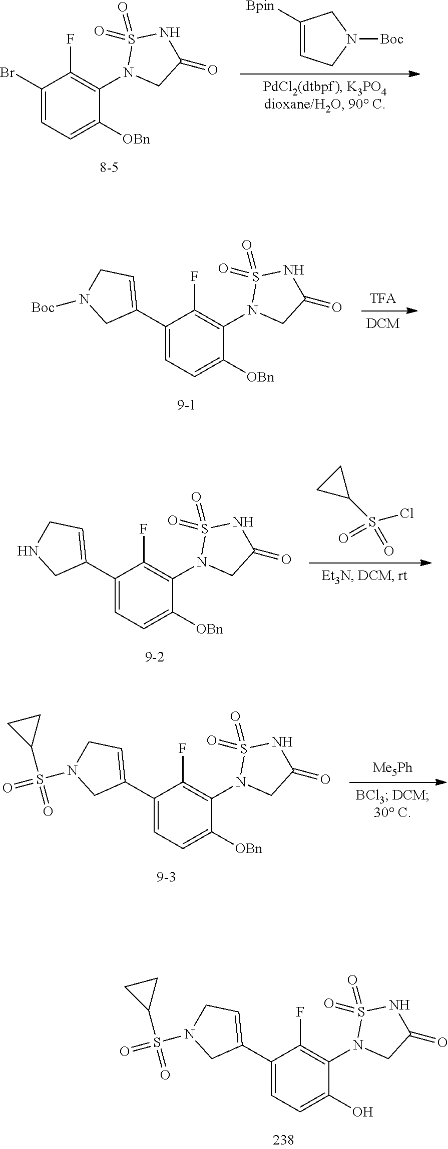

To a mixture of compound 8-5 (700 mg, 1.69 mmol, 1.0 eq), tert-butyl 3-(4,4,5,5-tetramethyl-1,3,2-dioxaborolan-2-yl)-2,5-dihydro-1H-pyrrole-1-carboxylate (550 mg, 1.86 mmol, 1.1 eq) and K₃PO₄ (1.08 mg, 5.08 mmol, 3.0 eq) in dioxane (4 mL) and H₂O (1.0 mL) was added PdCl₂(dtbpf) (110 mg, 0.17 mmol, 0.1 eq). The reaction mixture was stirred at 90° C. under N₂ atmosphere for 16 h. The reaction mixture was concentrated under reduced pressure. The residue was purified by column chromatography on silica gel eluting with MeOH/DCM (0-30%) to give compound 9-1 (780 mg). ESI-MS m/z: 502.2 [M−H]⁻.

To a mixture of compound 9-1 (780 mg, 1.55 mmol, 1.0 eq) in DCM (3 mL) was added TFA (885 mg, 7.76 mmol, 5.0 eq). The reaction mixture was stirred at rt for 3 h. The reaction mixture was concentrated under reduce pressure to afford compound 9-2 (860 mg). ESI-MS m/z: 402.2 [M−H]⁻.

To a mixture of compound 9-2 (260 mg, 0.65 mmol, 1.0 eq) in DCM (2 mL) was added Et₃N (130 mg, 1.29 mmol, 2.0 eq). The mixture was stirred at 0° C. for 10 min, then cyclopropanesulfonyl chloride (110 mg, 0.78 mmol, 1.2 eq) was added. The mixture was stirred at rt under N₂ for 16 h. The residue was purified by column chromatography on silica gel eluting with MeOH/DCM (0-30%) to obtain compound 9-3 (300 mg). ESI-MS m/z: 506.1 [M−H]⁻.

To a mixture of compound 9-3 (60 mg, 0.12 mmol, 1.0 eq) and Me₅Ph (53 mg, 0.358 mmol, 3.0 eq) in DCM (2 mL) was added BCl₃ (2 mL) at −78° C. After addition, the mixture was stirred at 30° C. for 2 h under N₂ atmosphere. The mixture was concentrated and quenched by NH₃-MeOH dropwise at −78° C. to pH-8. The reaction mixture was filtered and concentrated. The residue was purified by prep-HPLC (CAN/H₂O/NH₃·H₂O) to afford compound 238 (7.8 mg). ESI-MS m/z: 416.1 [M−H]⁻; ¹H NMR (400 MHz, DMSO-d6): δ 7.19 (t, J=8.6 Hz, 1H), 6.70 (d, J=8.6 Hz, 1H), 6.20 (s, 1H), 4.48 (s, 2H), 4.31 (s, 2H), 3.98 (s, 2H), 2.84-2.72 (m, 1H), 1.02-0.95 (m, 4H).

### Example 1j: Synthesis of 5-(2-fluoro-6-hydroxy-3-(3-(3,3,3-trifluoropropyl)-1H-pyrazol-5-yl)phenyl)-1,2,5-thiadiazolidin-3-one 1,1-dioxide (Compound 415)

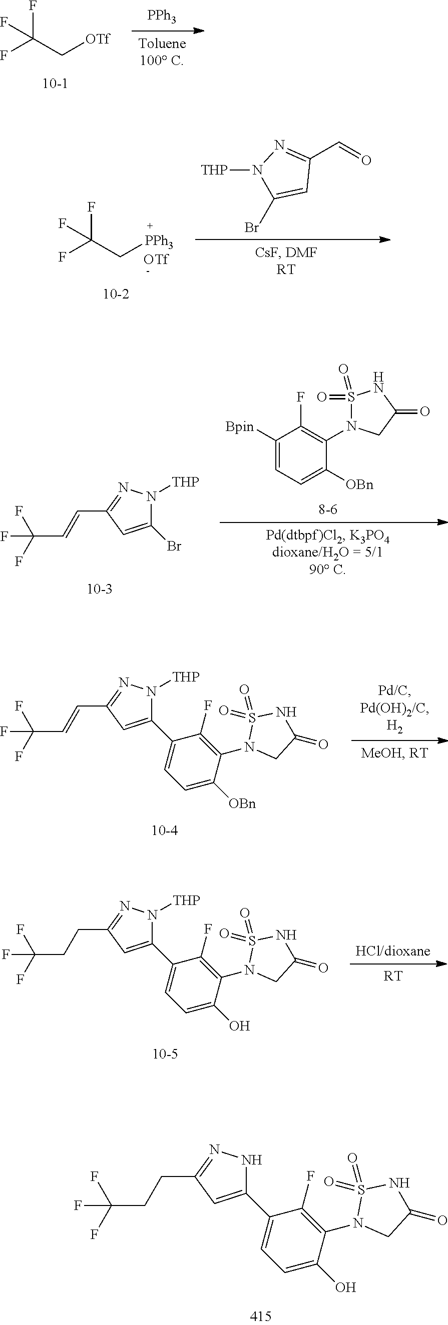

A mixture of compound 10-1 (1.0 g, 4.3 mmol, 1.0 eq) and PPh₃ (1.7 g, 1.7 mmol, 1.5 eq) in toluene (10 mL) was stirred at 100° C. overnight. The mixture was concentrated in vacuo to give compound 10-2 (3 g), which was used in the next without purification.

A mixture of compound 10-2 (2 g, crude), 5-bromo-1-(tetrahydro-2H-pyran-2-yl)-1H-pyrazole-3-carbaldehyde (500 mg, 1.94 mmol, 1.0 eq) and CsF (1.48 g, 9.70 mmol, 5 eq) in DMF (15 mL) was stirred under N₂ at RT for 1.5 h. The mixture was diluted with water (50 mL) and extracted with EtOAc (30 mL×3), and the organic phases were combined. The organic phase was washed with brine (100 mL), dried over anhydrous Na₂SO₄, and concentrated in vacuo. The residue was purified by column (EtOAc/PE=1/10) to afford compound 10-3 (130 mg). ¹H NMR (400 MHz, CDCl₃): δ 7.20 (ddd, J=16.0, 4.0, 2.0 Hz, 1H), 6.54 (s, 1H), 6.17-6.08 (m, 11H), 5.40 (dd, J=9.2, 2.8 Hz, 11H), 4.06-3.95 (m, 11H), 4.05-3.96 (m, 11H), 2.41-2.27 (m, 11H), 2.19-2.06 (m, 11H), 2.06-1.97 (m, 1H), 1.76-1.58 (m, 311).

A mixture of compound 10-3 (112 mg, 0.34 mmol, 2.0 eq), compound 8-6 (80 mg, 0.17 mmol, 1.0 eq), Pd(dtbpf)Cl₂ (13 mg, 0.02 mmol, 0.1 eq) and K₃PO₄ (108 mg, 0.51 mmol, 3 eq) in dioxane/H₂O=5/1 (3 mL) was stirred under nitrogen at 90° C. overnight. The mixture was concentrated in vacuo and the residue was purified by column (DCM/MeOH=3/1) to afford compound 10-4 (100 mg). ESI-MS m/z: 579.2 [M−H]⁻.

A mixture of compound 10-4 (100 mg, 0.17 mmol, 1.0 eq), Pd/C (10 mg) and Pd (OH)₂/C (10 mg) in MeOH (5 mL) was stirred at RT under H₂ (30 psi) for 3 h. The mixture was filtered, and the filtrate was concentrated in vacuo to afford compound 10-5 (50 mg). ESI-MS m/z: 491.2 [M−H]⁻.

A mixture of compound 10-5 (50 mg, 0.1 mmol, 1.0 eq) and 4M HCl in dioxane (3 mL) was stirred at RT for 1 h. The mixture was concentrated in vacuo and purified by prep-HPLC to afford compound 415 (12.6 mg). ESI-MS m/z: 407.2 [M−H]⁻; ¹H NMR (400 MHz, CD₃OD): δ 7.58 (s, 1H), 6.83 (d, J=8.8 Hz, 1H), 6.51 (s, 1H), 4.34 (s, 2H), 3.03-2.86 (m, 2H), 2.66-2.51 (m, 2H).

### Example 1k: Synthesis of 5-(2-fluoro-6-hydroxy-3-(1-isopentyl-3-oxo-2,3-dihydro-1H-pyrazol-4-yl)phenyl)-1,2,5-thiadiazolidin-3-one 1,1-dioxide (Compound 444)

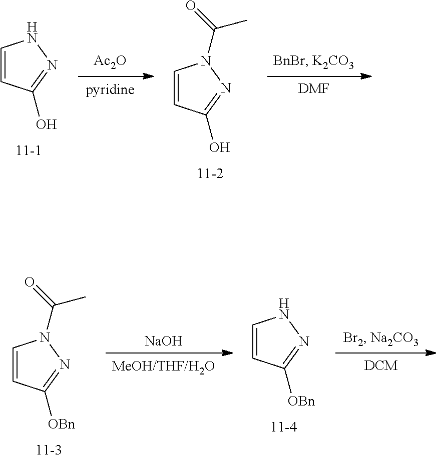

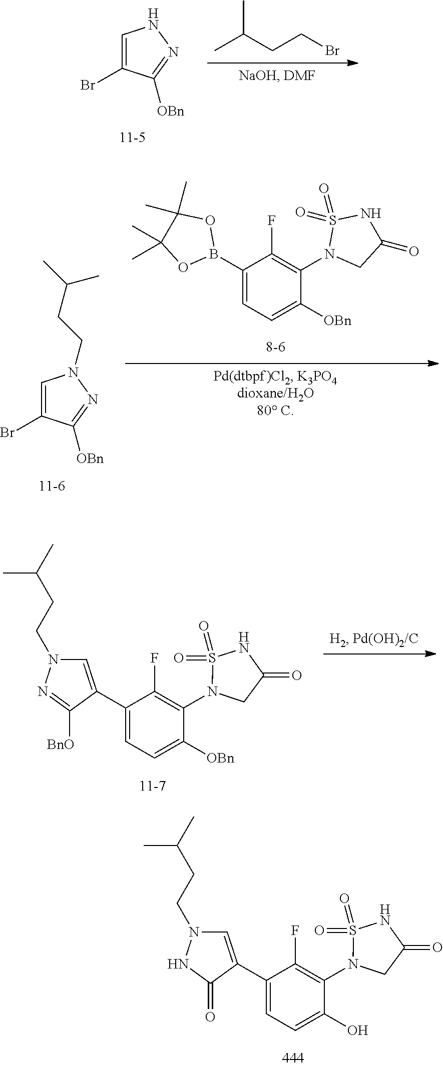

A mixture of 1H-pyrazol-5-ol (11-1) (5.00 g, 59.11 mmol) and pyridine (25 mL, 309.1 mmol) was stirred at 95° C., then a solution of acetic anhydride (5.6 mL, 59.35 mmol) in pyridine (10 mL, 123.6 mmol) was added dropwise over a period of 3 minutes. The mixture was then stirred at 95° C. for an additional 3 h. The solvents were removed under reduced pressure. The solid residue was triturated in 40 mL of diethyl ether, filtered, washed with diethyl ether and dried to afford compound 11-2 (7.1 g). ESI-MS m/z: 127.2 [M+H]⁺; ¹H NMR (400 MHz, DMSO-d6): δ 10.95 (s, 1H), 8.13 (d, J=2.9 Hz, 1H), 6.01 (d, J=2.9 Hz, 1H), 2.49 (s, 3H).

To a mixture of compound 11-2 (7.1 g, 56.29 mmol, 1.0 eq) and K₂CO₃ (12 g, 84.44 mmol, 1.5 eq) in DMF (70 mL) was added BnBr (14.85 g, 84.44 mmol, 1.5 eq) at RT. After addition, the mixture was stirred at RT for 16 h under Ar. The reaction mixture was diluted with EA (100 mL) and washed with H₂O (100 mL×3), then washed with brine (100 mL×3). The organic layer was dried with Na₂SO₄, filtered and concentrated. The residue was purified by column chromatography on silica gel eluting with PE/EA (0˜20%) to afford compound 11-3 (7.5 g). ESI-MS m/z: 217.1 [M+H]⁺.

To a mixture of compound 11-3 (7.5 g, 34.68 mmol, 1.0 eq) in THF (45 mL) and MeOH (30 mL) was added 10% NaOH solution (15 mL). After addition, the mixture was stirred at RT for 16 h under Ar. The reaction mixture was concentrated in vacuo. The residue was diluted with EtOH (20 mL) and H₂O (50 mL), then extracted with EA (50 mL×3). The combined organic layer was dried with Na₂SO₄, filtered, and concentrated under reduced pressure to afford compound 11-4 (5.6 g). ESI-MS m/z: 175.2 [M+H]⁺.

To a mixture of compound 11-4 (1.05 g, 6.14 mmol, 1.0 eq) and Na₂CO₃ (640 mg, 6.14 mmol, 1.0 eq) in DCM (10 mL) was added Br₂ (0.31 mL, 6.14 mmol, 1.0 eq) at 0° C. After addition, the mixture was stirred at 0° C. for 1 h under Ar. The reaction mixture was quenched with Na₂S₂O₃ aq (20 mL), diluted with H₂O (20 mL), and then extracted with DCM (30 mL×3). The combined organic layer was dried with Na₂SO₄, filtered and concentrated. The residue was purified by column chromatography on silica gel eluting with PE/EA (0-20%) to afford compound 11-5 (1 g). ESI-MS m/z: 255.0 [M+H]⁺.

A mixture of compound 11-5 (500 mg, 1.98 mmol, 1.0 eq), 1-bromo-3-methylbutane (597 mg, 3.95 mmol, 2.0 eq) and NaOH (158 mg, 3.95 mmol, 2.0 eq) in DMF (7 mL) was stirred at RT for 16 h under Ar. The reaction mixture was diluted with EA (30 mL) and washed with brine (10 mL×3). The organic layer was dried with Na₂SO₄, filtered, and concentrated under reduced pressure. The residue was purified by column chromatography on silica gel eluting with PE/EA (0-20%) to afford compound 11-6 (580 mg). ESI-MS m/z: 325.0 [M+H]⁺.

A mixture of compound 11-6 (126 mg, 0.39 mmol, 1.5 eq), compound 8-6 (120 mg, 0.26 mmol, 1.0 eq), PdCl₂(dtbpf) (26 mg, 0.039 mmol, 0.15 eq), and K₃PO₄ (165 mg, 0.78 mmol, 3.0 eq) in dioxane (2 mL) and H₂O (0.5 mL) was stirred at 80° C. under Ar for 16 h. The reaction mixture was purified directly by column chromatography on silica gel eluting with EA/MeOH (0-100%) to afford compound 11-7 (120 mg). ESI-MS m/z: 577.2 [M−H]⁻.

To a mixture of compound 11-7 (120 mg, 0.21 mmol, 1.0 eq) in MeOH was added Pd(OH)₂ (6 mg, 0.04 mmol, 0.2 eq). After addition, the mixture was stirred under H₂ (30 psi) at RT for 2 h. The reaction mixture was filtered, concentrated and the residue was purified by prep-HPLC (CAN/H₂O/NH₃·H₂O) to afford compound 444 (26.0 mg). ESI-MS m/z: 397.0 [M−H]⁻; ¹H NMR (400 MHz, CD₃OD): δ 7.76 (t, J=8.7 Hz, 1H), 7.64 (d, J=3.3 Hz, 1H), 6.73 (dd, J=8.8, 1.5 Hz, 1H), 4.34 (s, 2H), 3.99-3.94 (m, 2H), 1.73-1.67 (m, 2H), 1.60-1.51 (m, 1H), 0.96-0.93 (m, 6H).

### Example 1l: Synthesis of 5-(3-(5-cyclopropyl-4H-1,2,4-triazol-3-yl)-2-fluoro-6-hydroxyphenyl)-1,2,5-thiadiazolidin-3-one 1,1-dioxide (Compound 235)

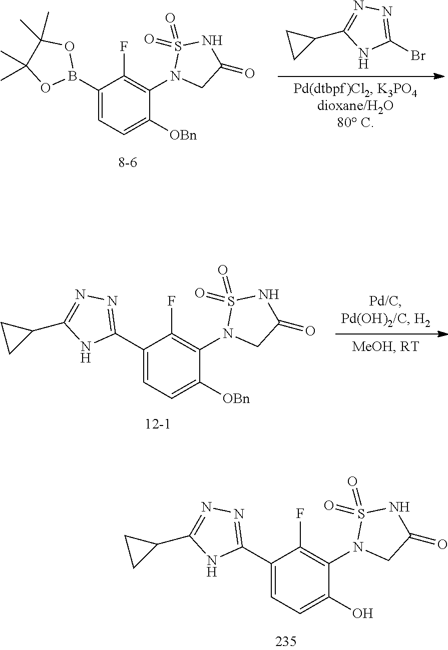

A mixture of compound 8-6 (100 mg, 0.53 mmol, 1.0 eq), 3-bromo-5-cyclopropyl-4H-1,2,4-triazole (123 mg, 0.26 mmol, 0.5 eq), Pd(dtbpf)Cl₂ (35 mg, 0.053 mmol, 0.1 eq) and K₃PO₄ (337 mg, 1.59 mmol, 3 eq) in dioxane/H₂O=5/1 (3 mL) was stirred under nitrogen at 80° C. overnight. The mixture was concentrated in vacuo and the residue was purified by column (DCM/MeOH=3/1) to afford compound 12-1 (70 mg). ESI-MS m/z: 442.1 [M−H]⁻.

A solution of compound 12-1 (60 mg, 0.14 mmol, 1.0 eq), Pd/C (15 mg) and Pd (OH)₂/C (15 mg) in MeOH (5 mL) was stirred under H₂ (30 psi) at RT for 3 h. The mixture was filtered, and the filtrate was concentrated in vacuo. The residue was purified by prep-HPLC to afford compound 235 (2.30 mg). ESI-MS m/z: 352.1 [M−H]⁻; ¹H NMR (400 MHz, CD₃OD): δ 7.75 (t, J=8.4 Hz, 1H), 6.85 (dd, J=8.8, 1.2 Hz, 1H), 4.32 (s, 2H), 2.11-2.03 (m, 1H), 1.07-0.99 (m, 4H).

### Example 1m: Synthesis of 5-(2-fluoro-6-hydroxy-3-(2-isopentyl-1H-imidazol-5-yl)phenyl)-1,2,5-thiadiazolidin-3-one 1,1-dioxide (Compound 334)

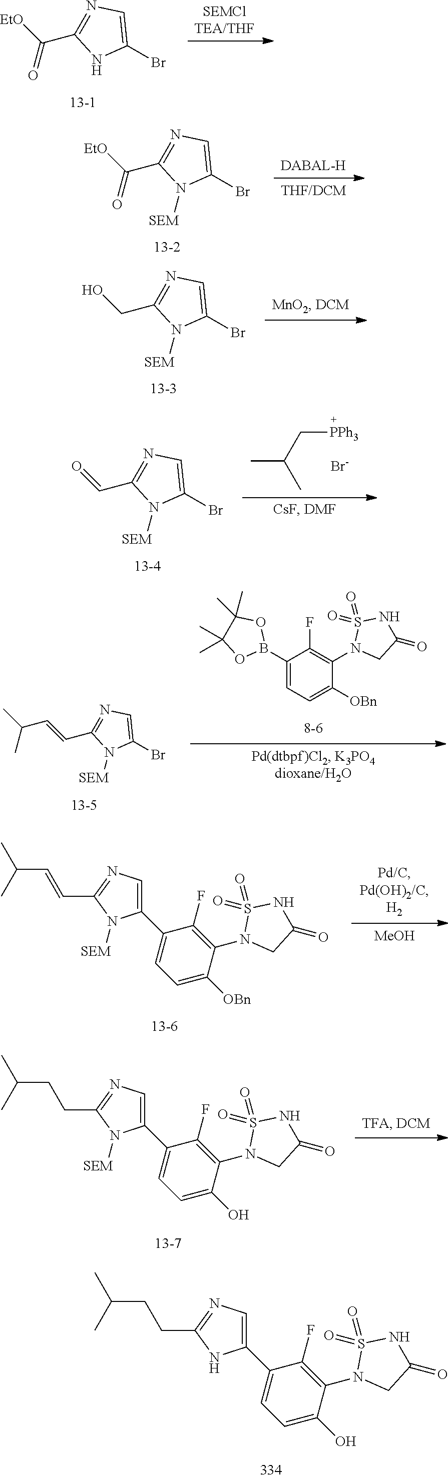

A mixture of compound 13-1 (700 mg, 3.2 mmol, 1.0 eq), SEMC1 (1.07 g, 6.4 mmol, 2 eq) and TEA (970 mg, 9.6 mmol, 3 eq) in THF (10 mL) was stirred at RT overnight. The mixture was diluted with water (40 mL), extracted with EtOAc (20 mL×3), and the organic phases were combined. The organic phase was washed with brine (60 mL), dried over anhydrous Na₂SO₄, and concentrated in vacuo to give compound 13-2 (1.3 g), which was used in the next step without further purification.

To a mixture of compound 13-2 (300 m) in DCM (10 mL) was added 1 M DABAL-H in THF (1 mL, 1 mmol, 1 eq) at 0° C. under N₂. The mixture was stirred at RT for 1 h. The mixture was quenched with ice-water (10 mL) and filtered. The filtrate was extracted with DCM (3×10 mL). The combined organic layer was dried over anhydrous Na₂SO₄ and concentrated in vacuo to afford compound 13-3 (250 mg), which was used in the next step without further purification.

To a mixture of compound 13-3 (250 mg) in DCM (10 mL) was added MnO₂ (710 mg, 8.2 mmol, 10 eq) at RT. The mixture was stirred at 40° C. overnight. This mixture was filtered, and the filtrate was concentrated in vacuo to afford compound 13-4 (113 mg), which was used in the next step without further purification. ¹H NMR (400 MHz, CDCl₃): δ 9.74 (d, J=18.4 Hz, 1H), 7.43-7.26 (m, 1H), 5.81 (d, J=30.4 Hz, 2H), 3.62 (d, J=8.4 Hz, 2H), 1.12 (d, J=1.6 Hz, 2H), 0.02 (d, J=1.2 Hz, 9H).

A mixture of compound 13-4 (110 mg, 0.36 mmol, 1 eq), isobutyltriphenylphosphonium bromide (287 mg, 0.72 mmol, 2 eq) and CsF (164 mg, 1.08 mmol, 3 eq) in DMF (3 mL) was stirred under nitrogen at RT for 2 h. The mixture was diluted with water (10 mL) and extracted with EtOAc (10 mL×3). The combined organic layer was washed with brine (30 mL), dried over anhydrous Na₂SO₄ and concentrated in vacuo to afford compound 13-5 (85 mg), which was used in the next step without further purification.

A mixture of compound 13-5 (85 mg, 0.25 mmol, 1.0 eq), compound 8-6 (116 mg, 0.25 mmol, 1.0 eq), Pd(dtbpf)Cl₂ (16 mg, 0.025 mmol, 0.1 eq) and K₃PO₄ (159 mg, 0.75 mmol, 3 eq) in dioxane/H₂O=5/1 (3 mL) was stirred under nitrogen at 90° C. overnight. The mixture was concentrated in vacuo and the residue was purified by column (DCM/MeOH=3/1) to afford compound 13-6 (80 mg). ESI-MS m/z: 599.4 [M−H]⁻.

A solution of compound 13-6 (80 mg, 0.13 mmol, 1.0 eq), Pd/C (15 mg) and Pd (OH)₂/C (15 mg) in MeOH (5 mL) was stirred under H₂ (30 psi) at RT for 3 h. The mixture was filtered, and the filtrate was concentrated in vacuo to afford compound 13-7 (50 mg). ESI-MS m/z: 511.3 [M−H]⁻.

To a mixture of compound 13-7 (50 mg, 0.1 mmol, 1.0 eq) in DCM (1 mL) was added TFA (1 mL). The mixture was stirred at RT for 1 h. The mixture was concentrated in vacuo and the residue was purified by prep-HPLC to afford compound 334 (6.2 mg). ESI-MS m/z: 381.0 [M−H]⁻; ¹H NMR (400 MHz, CD₃OD): δ 7.55 (t, J=8.4 Hz, 1H), 7.51 (d, J=2.0 Hz, 1H), 6.86 (d, J=8.8 Hz, 1H), 4.31 (s, 2H), 2.98-2.91 (m, 2H), 1.74-1.60 (m, 3H), 0.99 (d, J=6.4 Hz, 6H).

### Example 1n: Synthesis of N-(4-(3-(1,1-dioxido-4-oxo-1,2,5-thiadiazolidin-2-yl)-2-fluoro-4-hydroxyphenyl)cyclohexyl) cyclopropanesulfonamide (Compound 450)

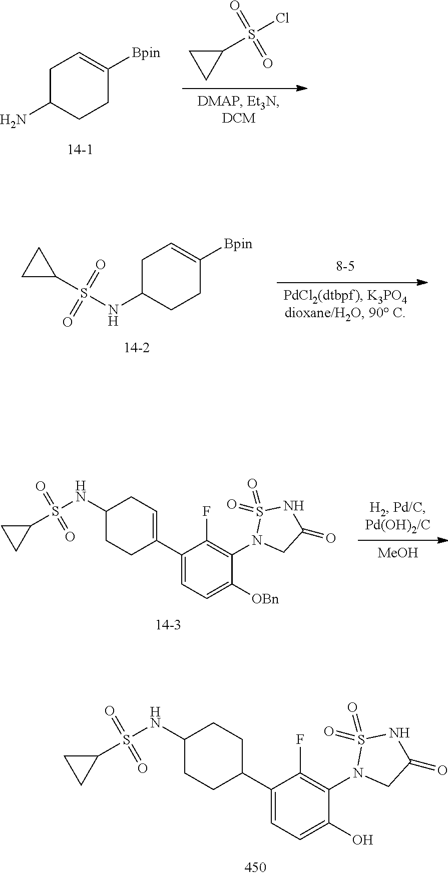

To a cooled (0° C.) mixture of compound 14-1 (200 mg, 0.90 mmol, 1.0 eq.), DMAP (11 mg, 0.090 mmol, 0.1 eq.) and Et₃N (0.4 mL, 0.27 mmol, 3.0 eq.) in DCM (5 mL) was added a solution of cyclopropanesulfonyl chloride (152 mg, 1.10 mmol, 1.2 eq.) in DCM (1 mL) dropwise. After addition, the reaction mixture was stirred at room temperature for 16 h. The reaction mixture was treated with water (20 mL) and extracted with DCM (10 mL×3). The combined organic layers were washed with brine (15 mL), dried over anhydrous Na₂SO₄, filtered and concentrated. The residue was purified by column chromatography on silica gel eluting with MeOH in DCM (0-5%) to obtain compound 14-2 (158 mg). ESI-MS m/z: 328.0 [M+H]⁺.

To a mixture of compound 14-2 (158 mg, 0.39 mmol, 2.0 eq.), compound 8-5 (80 mg, 0.19 mmol, 1.0 eq.), and K₃PO₄ (121 mg, 0.57 mmol, 3.0 eq.) in dioxane (4 mL) and H₂O (1 mL) was added PdCl₂(dtbpf) (12 mg, 0.019 mmol, 0.1 eq.). The mixture was stirred at 90° C. under Ar for 16 h, then cooled and concentrated in vacuo. The residue was purified by column chromatography on silica gel eluting with MeOH in EtOAc (0-30%) to obtain compound 14-3 (109 mg). ESI-MS m/z: 534.2 [M−H]⁻.

To a solution of compound 14-3 (89 mg, 0.17 mmol, 1.0 eq.) in MeOH (30 mL) were added Pd/C (15 mg, 10% wt) and Pd(O11)₂(15 mg, 20% wt), and the mixture was stirred at room temperature under H₂ overnight. The reaction mixture was filtered and the filtrate concentrated to give crude product, which was purified by prep-HPLC (CH₃CN+0.1% NH₃·H₂O) to obtain compound 450 (19.62 mg). ESI-MS m/z: 446.2 [M−H]⁻; ¹H NMR (400 MHz, methanol-d₄): δ 7.19 (t, J=8.4 Hz, 1H), 6.74-6.64 (m, 1H), 4.26 (s, 2H), 2.81 (dd, J=31.4, 10.0 Hz, 1H), 2.55 (td, J=7.4, 3.9 Hz, 1H), 2.27-1.32 (m, 9H), 1.10-0.95 (m, 4H).

### Example 1o: Synthesis of 5-(2-fluoro-6-hydroxy-3-(1-isopentylpiperidin-3-yl)phenyl)-1,2,5-thiadiazolidin-3-one 1,1-dioxide (Compound 198)

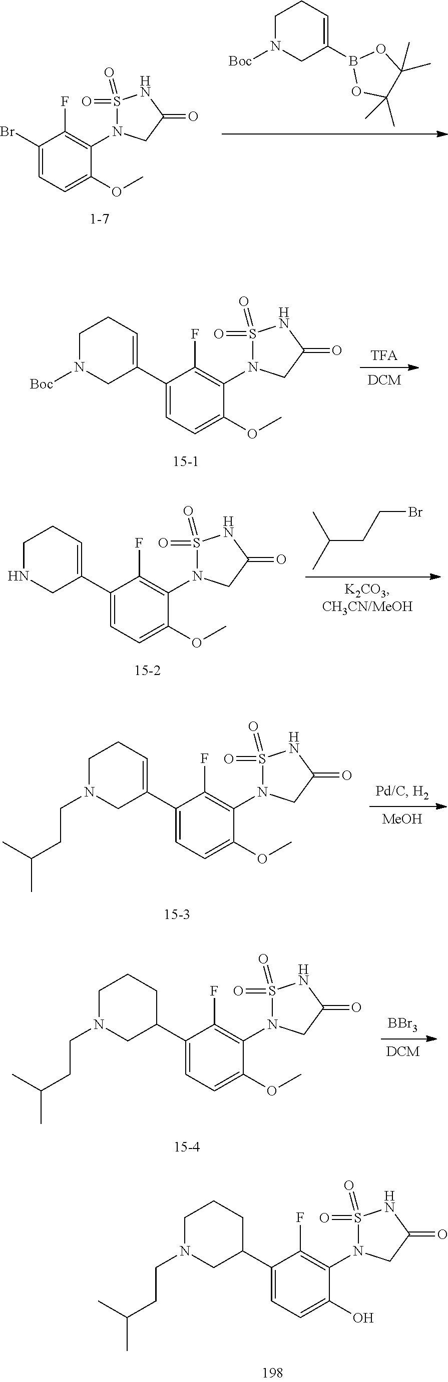

A reaction mixture of compound 1-7 (150 mg, 0.443 mmol, 1.0 eq.), tert-butyl 5-(4,4,5,5-tetramethyl-1,3,2-dioxaborolan-2-yl)-3,6-dihydropyridine-1(2H)-carboxylate (274 mg, 0.887 mmol, 2.0 eq.), Pd(dtbpf)Cl₂ (29 mg, 0.0443 mmol, 0.1 eq.) and K₃PO₄ (282 mg, 1.329 mmol, 0.1 eq.) in dioxane (5 mL) and H₂O (1 mL) was stirred at 90° C. under N₂ for 16 hours. The reaction mixture was concentrated under reduced pressure, then purified by column chromatography on silica gel eluting with MeOH/EA (0/1-1/3) to give compound 15-1 (160 mg). ESI-MS m/z: 440.2 [M−H]⁻.

A solution of compound 15-1 (160 mg, 0.363 mmol, 1.0 eq.) in DCM (1.5 ml) and TFA (0.5 ml) was stirred at rt for 1 hour, then concentrated in vacuo. The residue was adjusted pH to 8-9 with ammonia and concentrated under reduced pressure. The residue was purified by column chromatography on silica gel eluting with MeOH/ethyl acetate (0/1-1/3) to obtain compound 15-2 (130 mg). ESI-MS m/z: 340.2 [M−H]⁻.

To a solution of compound 15-2 (130 mg, 0.381 mmol, 1.0 eq.) in MeOH/MeCN (2 mL/2 mL) was added 1-bromo-3-methylbutane (229 mg, 1.525 mmol, 4.0 eq.) and K₂CO₃ (315 mg, 2.286 mmol, 6.0 eq.). The reaction mixture was stirred at 70° C. for 16 h under N₂, then concentrated under reduced pressure. The residue was purified by column chromatography on silica gel eluting with MeOH/ethyl acetate (0/1-1/3) to obtain compound 15-3 (140 mg). ESI-MS m/z: 410.2 [M−H]⁻.

To a solution of compound 15-3 (140 mg, 0.34 mmol, 1.0 eq.) in MeOH (5 mL) was added Pd/C (30 mg, 10% wt, wet). The reaction mixture was stirred at rt for 3 h under H₂ (30 psi), then filtered and the filter cake washed with MeOH (50 mL). The filtrate was concentrated under reduced pressure to obtain crude compound 15-4 (130 mg), which was used for the next step without further purification. ESI-MS m/z: 412.3 [M−H]⁻.

To a solution of compound 15-4 (130 mg, 0.315 mmol, 1.0 eq.) in DCM (1 mL) was added BBr₃ (1 mL) at −78° C. under N₂. The reaction mixture was stirred at room temperature for 2 h under N₂, then quenched with MeOH and adjusted to pH=8-9 with aq. NH₄OH at −78° C. The mixture was concentrated under reduced pressure and the residue dissolved in DMF (3 ml) and filtered. The filtrate was purified by pre-HPLC separation to afford compound 198 (21.92 mg). ESI-MS m/z: 398.3 [M−H]⁻; ¹H NMR (400 MHz, DMSO-d6): δ 9.46 (s, 1H), 9.18 (br s, 1H), 7.09 (t, J=8.4 Hz, 1H), 6.70 (d, J=8.4 Hz, 1H), 3.96 (s, 2H), 3.60-3.35 (m, 2H), 3.18-2.85 (m, 5H), 1.98-1.51 (m, 7H), 0.90 (d, J=6.0 Hz, 6H).

### Example 1p: Synthesis of N-(3′-(1,1-dioxido-4-oxo-1,2,5-thiadiazolidin-2-yl)-2,2′-difluoro-4′-hydroxy-[1,1′-biphenyl]-4-yl)cyclopropanesulfonamide (Compound 438)

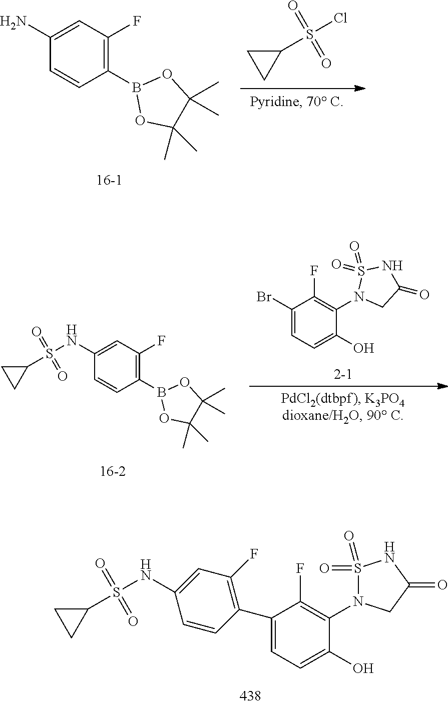

A mixture of compound 16-1 (250 mg, 1.05 mmol, 1.0 eq) and cyclopropanesulfonyl chloride (294 mg, 2.10 mmol, 2.0 eq) in pyridine (10 mL) was stirred at 70° C. under Ar overnight. The reaction mixture was concentrated in vacuo and the residue was purified by column chromatography on silica gel eluting with EtOAc in petroleum ether (0-30%) to obtain the desired product 16-2 (200 mg). 1H NMR (400 MHz, DMSO-d₆): δ 10.25 (s, 1H), 7.58 (t, J=7.6 Hz, 1H), 7.06 (dd, J=8.4, 1.6 Hz, 1H), 6.96 (dd, J=11.2, 1.6 Hz, 11), 2.75 (tt, J=7.2, 5.2 Hz, 11), 1.28 (s, 12H), 1.01-0.95 (m, 4H).

To a mixture of compound 16-2 (85 mg, 0.25 mmol, 1.0 eq.), compound 2-1 (80 mg, 0.25 mmol, 1.0 eq.) and K₃PO₄ (159 mg, 0.75 mmol, 3.0 eq.) in dioxane/H₂O (5 mL/1 mL) was added PdCl₂(dtbpf)(16 mg, 0.025 mmol, 0.1 eq.). The mixture was stirred at 90° C. under Ar overnight, then concentrated in vacuo and the residue purified by prep-HPLC (CH₃CN/H₂O+0.1% NH₄HCO₃) to obtain compound 438 (39.13 mg). ESI-MS m/z: 458.1 [M−H]⁻; 1H NMR (400 MHz, methanol-d₄) δ 7.33 (t, J=8.4 Hz, 1H), 7.21 (t, J=8.4 Hz, 1H), 7.16-7.09 (m, 2H), 6.81 (d, J=8.8 Hz, 1H), 4.31 (s, 2H), 2.70-2.56 (m, 1H), 1.15-0.95 (m, 4H).

### Example 1q: Synthesis of 5-(2-fluoro-6-hydroxy-3-(5-oxo-2,5-dihydrofuran-3-yl)phenyl)-1,2,5-thiadiazolidin-3-one 1,1-dioxide (Compound 513)

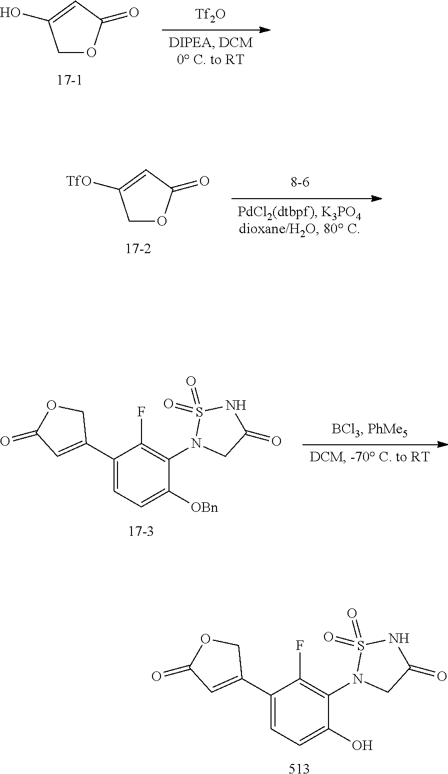

To a cooled (0° C.) solution of compound 17-1 (350 mg, 3.5 mmol, 1.0 eq.) and DIPEA (542 mg, 4.2 mmol, 1.2 eq) in DCM (5 mL) was added a solution of Tf₂O (1.18 g, 4.2 mmol, 1.2 eq) in DCM (1 mL) under Ar. After addition, the mixture was stirred at RT under Ar for 1 h. The reaction mixture was concentrated under reduced pressure. The residue was purified by column chromatography on silica gel eluting with EtOAc in petroleum ether (0-10%) to obtain compound 17-2 (400 mg). ESI-MS m/z: 233.0 [M+H]⁺.

To a mixture of compound 17-2 (400 mg, 1.7 mmol, 1.5 eq.), compound 8-6 (554 mg, 1.2 mmol, 1.0 eq.) and K₃PO₄(636 mg, 3.0 mmol, 2.5 eq.) in dioxane (20 mL) and H₂O (5 mL) was added PdCl₂(dtbpf) (118 mg, 0.18 mmol, 0.15 eq). The mixture was stirred at 80° C. under Ar for 3 h, then cooled and concentrated in vacuo. The residue was purified by column chromatography on silica gel eluting with MeOH in EtOAc (0 to 30%) to obtain compound 17-3 (540 mg). ESI-MS m/z: 417.0 [M−H]⁻.

To a cooled (−70° C.) solution of compound 17-3 (45 mg, 0.108 mmol, 1.0 eq.) in DCM (1.5 mL) was added PhMe₅ (47 mg, 0.323 mmol, 3.0 eq.), followed by the dropwise addition of BCl₃ (1 mL, 1M in DCM) under Ar. After addition, the mixture was stirred at RT for 3 h. The reaction was quenched with MeOH (10 mL) at −70° C. and adjusted to pH=8-9 with NH₃·H₂O, then concentrated under reduced pressure to dryness. The residue was purified by prep-HPLC (CH₃CN/H₂O+0.1% NH₄HCO₃) to obtain compound 513 (2.97 mg). ESI-MS m/z: 327.2 [M−H]⁻; ¹H NMR (400 MHz, methanol-d₄): δ 7.53 (t, J=8.4 Hz, 1H), 6.84 (d, J=9.2 Hz, 1H), 6.41 (s, 1H), 5.34 (s, 2H), 4.29 (s, 2H).

### Example 1r: Synthesis of 5-(3-(5-(cyclopropylmethyl)-2,5-dihydro-1H-pyrrol-3-yl)-2-fluoro-6-hydroxyphenyl)-1,2,5-thiadiazolidin-3-one 1,1-dioxide (Compound 522)

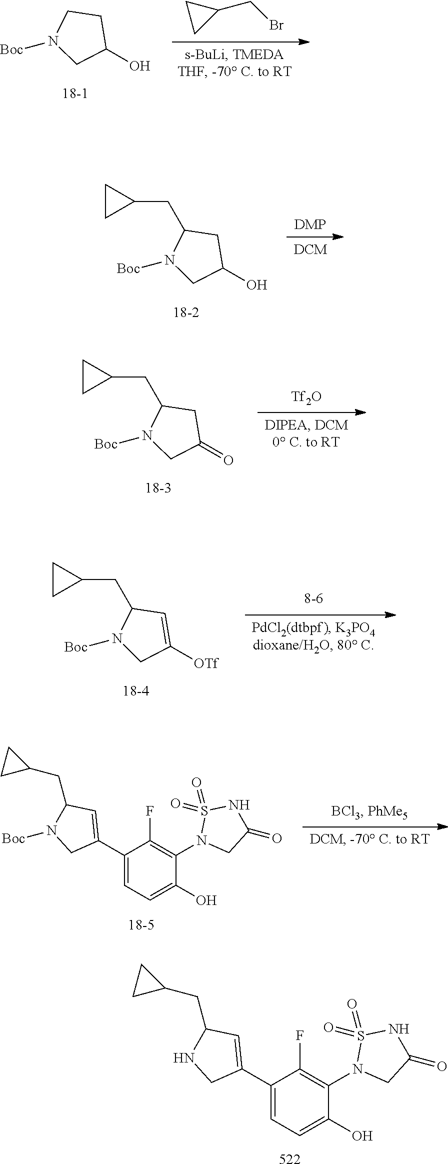

To a cooled (−70° C.) solution of compound 18-1 (1.00 g, 5.35 mmol, 1.0 eq.) and TMEDA (1.24 g, 10.7 mmol, 2.0 eq.) in THF (15 mL) was added s-BuLi (16.5 mL, 21.4 mmol, 1.3 M in hexane) dropwise under Ar. The reaction mixture was stirred at approximately −65° C. for 3 h, then a solution of (bromomethyl)cyclopropane (2.14 g, 11.76 mmol, 2.2 eq) in THF (5 mL) was added dropwise. After the addition, the reaction was warmed to room temperature (RT) naturally and stirred at this temperature for 16 h. The reaction was quenched with aq. NH₄Cl (sat. 120 mL) and extracted with EtOAc (80 mL×3). The combined organic layers were washed withed brine (100 mL), dried over anhydrous Na₂SO₄, filtered, and concentrated in vacuo. The residue was purified by column chromatography eluting with EtOAc in petroleum ether (0-30%) to obtain compound 18-2 (550 mg).

To a mixture of compound 18-2 (150 mg, 0.622 mmol, 1.0 eq.) in DCM (5 mL) was added Dess-Martin Reagent (DMP) (792 mg, 1.87 mmol, 3.0 eq.) in portions at 0° C. The resulting mixture was stirred at RT for 3 h, then treated with aq. NaHCO₃ (sat. 50 mL) and extracted with DCM (25 mL×3). The combined organic layers were washed with brine (40 mL), dried over anhydrous Na₂SO₄, filtered and concentrated. The residue was purified by column chromatography eluting with EtOAc in petroleum ether (0-20%) to obtain compound 18-3 (125 mg).

To a cooled (0° C.) solution of compound 18-3 (125 mg, 0.523 mmol, 1.0 eq.) and DIPEA (675 mg, 5.23 mmol, 10.0 eq.) in DCM (5 mL) was added Tf₂O (737 mg, 2.62 mmol, 5.0 eq.) dropwise under Ar. The resulting mixture was stirred at RT for 16 h, then treated with water (40 mL) and extracted with DCM (20 mL×3). The combined organic layers were washed with brine (40 mL), dried over anhydrous Na₂SO₄, filtered and concentrated. The residue was purified by column chromatography eluting with EtOAc in petroleum ether (0-20%) to obtain compound 18-4 (60 mg).

To a mixture of compound 18-4 (125 mg, 0.337 mmol, 1.0 eq.), compound 8-6 (171 mg, 0.370 mmol, 1.1 eq) and K₃PO₄ (214 mg, 1.01 mmol, 3.0 eq.) in dioxane (2 mL) and H₂O (0.5 mL) was added PdCl₂(dtbpf) (22 mg, 0.034 mmol, 0.1 eq.). The mixture was stirred at 80° C. under Ar for 16 h. The reaction mixture was concentrated in vacuo. The residue was purified by column chromatography eluting with MeOH in EtOAc (0-20%) to obtain compound 18-5 (60 mg). ESI-MS m/z: 556.2 [M−H]⁻.

To a cooled (−70° C.) mixture of compound 18-5 (90 mg, 0.161 mmol, 1.0 eq.) and PhMe₅ (72 mg, 0.484 mmol, 3.0 eq.) in DCM (1 mL) was added BCl₃ (1 mL, 1 M in DCM) dropwise under Ar. After addition, the mixture was stirred at RT for 2 h. The reaction mixture was quenched with MeOH (10 mL) at −70° C. and adjusted to pH=8-9 with NH₃·H₂O, then concentrated to dryness. The residue was purified by prep-HPLC (CH₃CN/H₂O+0.1% NH₄₁CO₃) to obtain compound 522 (1.28 mg). ESI-MS m/z: 366.0 [H−M]⁻; 1H NMR (400 MHz, DMSO-d₆): δ 7.26 (s, 1H), 6.73 (d, J=8.1 Hz, 1H), 6.27 (s, 1H), 4.53 (s, 1H), 4.30 (s, 2H), 3.97 (s, 2H), 1.87-1.49 (m, 2H), 0.84 (s, 1H), 0.49 (d, J=7.7 Hz, 2H), 0.18 (s, 2H).

### Example 1s: Synthesis of 5-(3-(7-azabicyclo[2.2.1]hept-2-en-2-yl)-2-fluoro-6-hydroxyphenyl)-1,2,5-thiadiazolidin-3-one 1,1-dioxide (Compound 475)

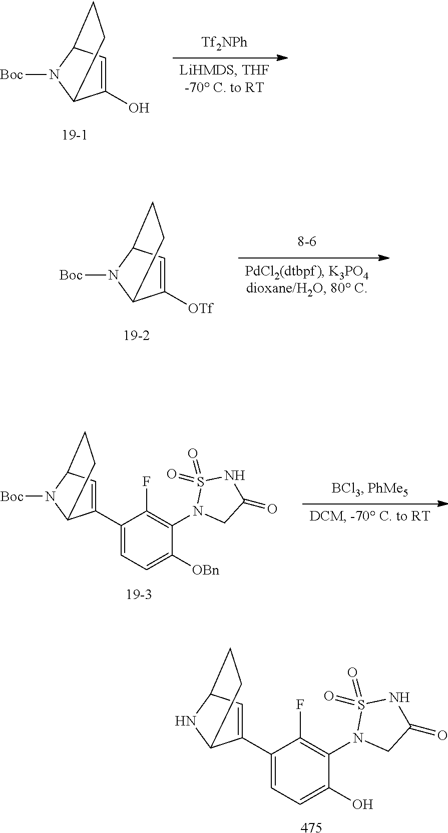

To a cooled (−70° C.) solution of compound 19-1 (300 mg, 1.4 mmol, 1.0 eq.) in THF (10 mL) was added LiHMDS (1M in THF, 2.8 mL, 2.8 mmol, 2 eq.) dropwise under Ar. The reaction mixture was stirred at approximately −65° C. for 1 h, then a solution of Tf₂NPh (762 mg, 2.1 mmol, 1.5 eq.) in THF (5 mL) was added dropwise. After addition, the reaction was stirred at RT for 1 h, then quenched with aq. NH₄Cl (sat. 40 mL) and extracted with EtOAc (20 mL×3). The combined organic layers were washed withed brine (50 mL), dried over anhydrous Na₂SO₄, filtered, and concentrated in vacuum. The residue was purified by column chromatography eluting with EtOAc in petroleum ether (0-30%) to obtain compound 19-2 (710 mg, crude) as a yellow solid, which was used for the next step without further purification.

To a mixture of compound 19-2 (97 mg, 0.28 mmol, 1.0 eq.), compound 8-6 (130 mg, 0.28 mmol, 1.0 eq.) and K₃PO₄ (178 mg, 0.84 mmol, 3.0 eq.) in dioxane (5 mL) and H₂O (1 mL) was added PdCl₂(dtbpf) (18 mg, 0.03 mmol, 0.1 eq.). The mixture was stirred at 80° C. under Ar for 16 h, then concentrated in vacuo and the residue purified by column chromatography eluting with MeOH in EtOAc (0-30%) to obtain desired product compound 19-3 (130 mg). ESI-MS m/z: 528.3 [M−H]⁻.

To a cooled (−70° C.) mixture of compound 19-3 (130 mg, 0.25 mmol, 1.0 eq.) and PhMe₅ (109 mg, 0.74 mmol, 3.0 eq.) in DCM (2 mL) was added BCl₃ (2 mL, 1 M in DCM) dropwise under Ar. After addition, the mixture was stirred at RT for 2 h. The reaction mixture was quenched with MeOH (12 mL) at −70° C. and adjusted to pH=8-9 with NH₃·H₂O, then concentrated to dryness. The residue was purified by prep-HPLC (CH₃CN/H₂O+0.1% NH₄HCO₃) to obtain compound 475 (1.90 mg). ESI-MS m/z: 338.0 [M−H]⁻; ¹H NMR (400 MHz, methanol-d₄): δ 7.37 (t, J=8.6 Hz, 1H), 6.79 (d, J=8.5 Hz, 1H), 6.56 (s, 1H), 5.16 (s, 1H), 4.78 (s, 1H), 4.28 (s, 2H), 2.18 (dd, J=6.7, 2.5 Hz, 2H), 1.68-1.57 (in, 2H).

The following compounds listed in Table 2 were synthesized utilizing one or more general schemes disclosed herein, including those above, following the specific examples exemplified above, or by methods generally known in the art.

TABLE 2

| No. | Structure | Chemical Name | [M − H]⁻ |
|---|---|---|---|
| 101 | 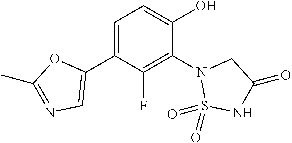 [SMILES: Cc1ncc(-c2ccc(O)c(N3CC(=O)NS3(=O)=O)c2F)o1] | 5-(2-fluoro-6-hydroxy-3-(2-methyloxazol-5- yl)phenyl)-1,2,5-thiadiazolidin-3-one 1,1-dioxide | 326.0 |
| 102 | 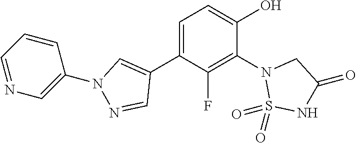 [SMILES: O=C1CN(c2c(O)ccc(-c3cnn(-c4cccnc4)c3)c2F)S(=O)(=O)N1] | 5-(2-fluoro-6-hydroxy-3-(1-(pyridin-3-yl)-1H- pyrazol-4-yl)phenyl)-1,2,5-thiadiazolidin-3-one 1,1- dioxide | 388.2 |
| 103 | 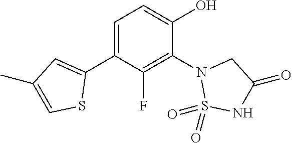 [SMILES: Cc1csc(-c2ccc(O)c(N3CC(=O)NS3(=O)=O)c2F)c1] | 5-(2-fluoro-6-hydroxy-3-(4-methylthiophen-2- yl)phenyl)-1,2,5-thiadiazolidin-3-one 1,1-dioxide | 341.0 |
| 104 | 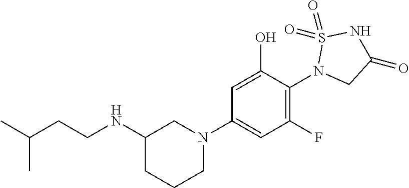 [SMILES: CC(C)CCNC1CCCN(c2cc(O)c(N3CC(=O)NS3(=O)=O)c(F)c2)C1] | 5-(2-fluoro-6-hydroxy-4-(3- (isopentylamino)piperidin-1-yl)phenyl)-1,2,5- thiadiazolidin-3-one 1,1-dioxide | 413.4 |
| 105 | 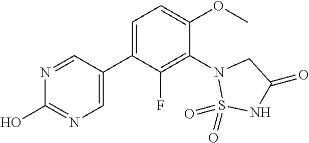 [SMILES: COc1ccc(-c2cnc(O)nc2)c(F)c1N1CC(=O)NS1(=O)=O] | 5-(2-fluoro-3-(2-hydroxypyrimidin-5-yl)-6- methoxyphenyl)-1,2,5-thiadiazolidin-3-one 1,1- dioxide | 353.2 |
| 106 | 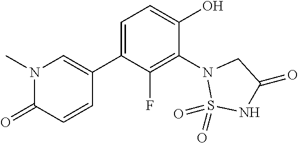 [SMILES: Cn1cc(-c2ccc(O)c(N3CC(=O)NS3(=O)=O)c2F)ccc1=O] | 5-(2-fluoro-6-hydroxy-3-(1-methyl-6-oxo-1,6- dihydropyridin-3-yl)phenyl)-1,2,5-thiadiazolidin-3- one 1,1-dioxide | 352.0 |
| 107 | 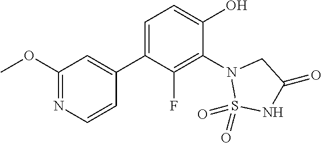 [SMILES: COc1cc(-c2ccc(O)c(N3CC(=O)NS3(=O)=O)c2F)ccn1] | 5-(2-fluoro-6-hydroxy-3-(2-methoxypyridin-4- yl)phenyl)-1,2,5-thiadiazolidin-3-one 1,1-dioxide | 352.2 |
| 108 | 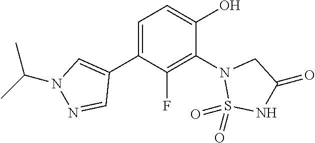 [SMILES: CC(C)n1cc(-c2ccc(O)c(N3CC(=O)NS3(=O)=O)c2F)cn1] | 5-(2-fluoro-6-hydroxy-3-(1-isopropyl-1H-pyrazol-4- yl)phenyl)-1,2,5-thiadiazolidin-3-one 1,1-dioxide | 352.9 |
| 109 | 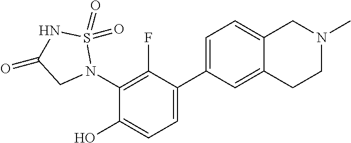 [SMILES: CN1CCc2cc(-c3ccc(O)c(N4CC(=O)NS4(=O)=O)c3F)ccc2C1] | 5-(2-fluoro-6-hydroxy-3-(2-methyl-1,2,3,4- tetrahydroisoquinolin-6-yl)phenyl)-1,2,5- thiadiazolidin-3-one 1,1-dioxide | 390.2 |
| 110 | 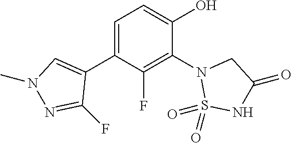 [SMILES: Cn1cc(-c2ccc(O)c(N3CC(=O)NS3(=O)=O)c2F)c(F)n1] | 5-(2-fluoro-3-(3-fluoro-1-methyl-1H-pyrazol-4-yl)- 6-hydroxyphenyl)-1,2,5-thiadiazolidin-3-one 1,1- dioxide | 343.2 |
| 111 | 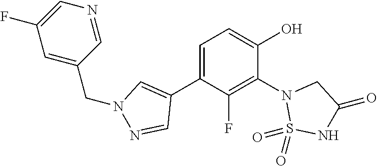 [SMILES: O=C1CN(c2c(O)ccc(-c3cnn(Cc4cncc(F)c4)c3)c2F)S(=O)(=O)N1] | 5-(2-fluoro-3-(1-((5-fluoropyridin-3-yl)methyl)-1H- pyrazol-4-yl)-6-hydroxyphenyl)-1,2,5-thiadiazolidin- 3-one 1,1-dioxide | 420.0 |
| 112 | 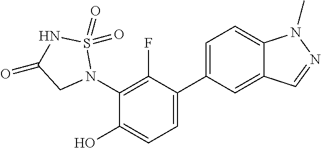 [SMILES: Cn1ncc2cc(-c3ccc(O)c(N4CC(=O)NS4(=O)=O)c3F)ccc21] | 5-(2-fluoro-6-hydroxy-3-(1-methyl-1H-indazol-5- yl)phenyl)-1,2,5-thiadiazolidin-3-one 1,1-dioxide | 375.0 |
| 113 | 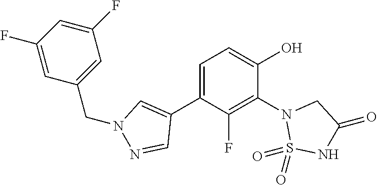 [SMILES: O=C1CN(c2c(O)ccc(-c3cnn(Cc4cc(F)cc(F)c4)c3)c2F)S(=O)(=O)N1] | 5-(3-(1-(3,5-difluorobenzyl)-1H-pyrazol-4-yl)-2- fluoro-6-hydroxyphenyl)-1,2,5-thiadiazolidin-3-one 1,1-dioxide | 437.1 |
| 114 | 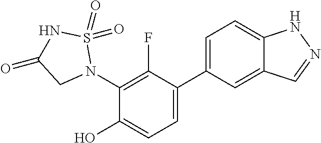 [SMILES: O=C1CN(c2c(O)ccc(-c3ccc4[nH]ncc4c3)c2F)S(=O)(=O)N1] | 5-(2-fluoro-6-hydroxy-3-(1H-indazol-5-yl)phenyl)- 1,2,5-thiadiazolidin-3-one 1,1-dioxide | 361.1 |
| 115 | 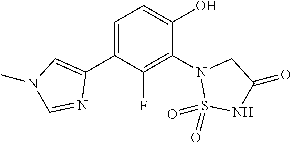 [SMILES: Cn1cnc(-c2ccc(O)c(N3CC(=O)NS3(=O)=O)c2F)c1] | 5-(2-fluoro-6-hydroxy-3-(1-methyl-1H-imidazol-4- yl)phenyl)-1,2,5-thiadiazolidin-3-one 1,1-dioxide | 325.0 |
| 116 | 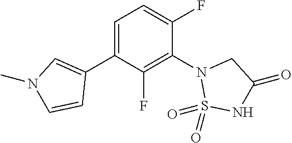 [SMILES: Cn1ccc(-c2ccc(F)c(N3CC(=O)NS3(=O)=O)c2F)c1] | 5-(2,6-difluoro-3-(1-methyl-1H-pyrrol-3-yl)phenyl)- 1,2,5-thiadiazolidin-3-one 1,1-dioxide | 325.9 |
| 117 | 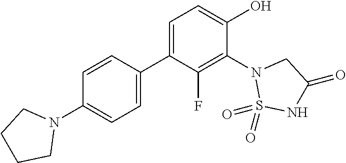 [SMILES: O=C1CN(c2c(O)ccc(-c3ccc(N4CCCC4)cc3)c2F)S(=O)(=O)N1] | 5-(2-fluoro-4-hydroxy-4′-(pyrrolidin-1-yl)-[1,1′- biphenyl]-3-yl)-1,2,5-thiadiazolidin-3-one 1,1- dioxide | 390.0 |
| 118 | 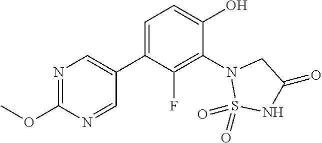 [SMILES: COc1ncc(-c2ccc(O)c(N3CC(=O)NS3(=O)=O)c2F)cn1] | 5-(2-fluoro-6-hydroxy-3-(2-methoxypyrimidin-5- yl)phenyl)-1,2,5-thiadiazolidin-3-one 1,1-dioxide | 353.0 |
| 119 | 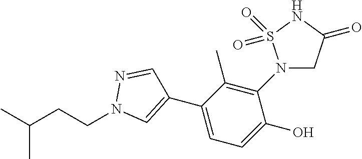 [SMILES: Cc1c(-c2cnn(CCC(C)C)c2)ccc(O)c1N1CC(=O)NS1(=O)=O] | 5-(6-hydroxy-3-(1-isopentyl-1H-pyrazol-4-yl)-2- methylphenyl)-1,2,5-thiadiazolidin-3-one 1,1-dioxide | 377.2 |
| 120 | 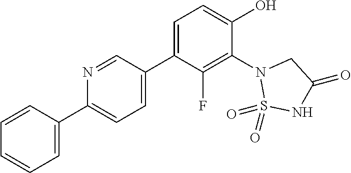 [SMILES: O=C1CN(c2c(O)ccc(-c3ccc(-c4ccccc4)nc3)c2F)S(=O)(=O)N1] | 5-(2-fluoro-6-hydroxy-3-(6-phenylpyridin-3- yl)phenyl)-1,2,5-thiadiazolidin-3-one 1,1-dioxide | 398.1 |
| 121 | 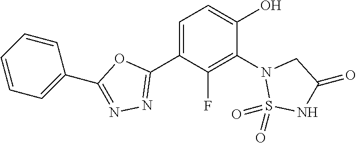 [SMILES: O=C1CN(c2c(O)ccc(-c3nnc(-c4ccccc4)o3)c2F)S(=O)(=O)N1] | 5-(2-fluoro-6-hydroxy-3-(5-phenyl-1,3,4-oxadiazol- 2-yl)phenyl)-1,2,5-thiadiazolidin-3-one 1,1-dioxide | 389.0 |
| 122 | 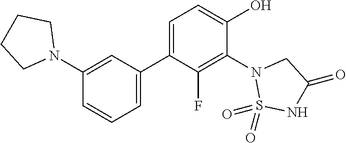 [SMILES: O=C1CN(c2c(O)ccc(-c3cccc(N4CCCC4)c3)c2F)S(=O)(=O)N1] | 5-(2-fluoro-4-hydroxy-3′-(pyrrolidin-1-yl)-[1,1′- biphenyl]-3-yl)-1,2,5-thiadiazolidin-3-one 1,1- dioxide | 390.0 |
| 123 | 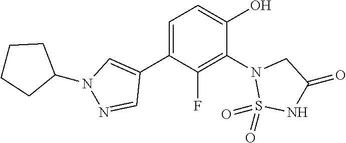 [SMILES: O=C1CN(c2c(O)ccc(-c3cnn(C4CCCC4)c3)c2F)S(=O)(=O)N1] | 5-(3-(1-cyclopentyl-1H-pyrazol-4-yl)-2-fluoro-6- hydroxyphenyl)-1,2,5-thiadiazolidin-3-one 1,1- dioxide | 379.0 |
| 124 | 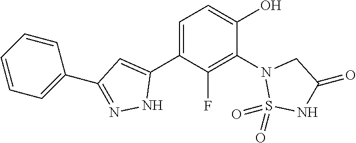 [SMILES: O=C1CN(c2c(O)ccc(-c3cc(-c4ccccc4)n[nH]3)c2F)S(=O)(=O)N1] | 5-(2-fluoro-6-hydroxy-3-(3-phenyl-1H-pyrazol-5- yl)phenyl)-1,2,5-thiadiazolidin-3-one 1,1-dioxide | 387.0 |
| 125 | 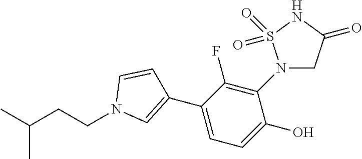 [SMILES: CC(C)CCn1ccc(-c2ccc(O)c(N3CC(=O)NS3(=O)=O)c2F)c1] | 5-(2-fluoro-6-hydroxy-3-(1-isopentyl-1H-pyrrol-3- yl)phenyl)-1,2,5-thiadiazolidin-3-one 1,1-dioxide | 380.3 |
| 126 | 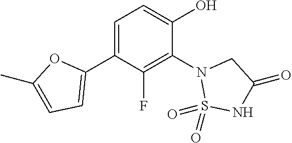 [SMILES: Cc1ccc(-c2ccc(O)c(N3CC(=O)NS3(=O)=O)c2F)o1] | 5-(2-fluoro-6-hydroxy-3-(5-methylfuran-2- yl)phenyl)-1,2,5-thiadiazolidin-3-one 1,1-dioxide | 325.0 |
| 127 | 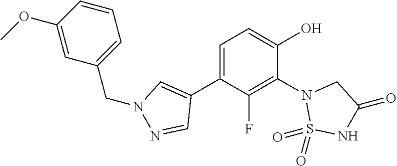 [SMILES: COc1cccc(Cn2cc(-c3ccc(O)c(N4CC(=O)NS4(=O)=O)c3F)cn2)c1] | 5-(2-fluoro-6-hydroxy-3-(1-(3-methoxybenzyl)-1H- pyrazol-4-yl)phenyl)-1,2,5-thiadiazolidin-3-one 1,1- dioxide | 431.2 |
| 128 | 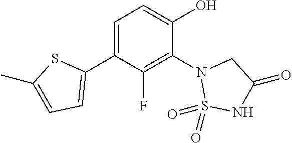 [SMILES: Cc1ccc(-c2ccc(O)c(N3CC(=O)NS3(=O)=O)c2F)s1] | 5-(2-fluoro-6-hydroxy-3-(5-methylthiophen-2- yl)phenyl)-1,2,5-thiadiazolidin-3-one 1,1-dioxide | 340.9 |
| 129 | 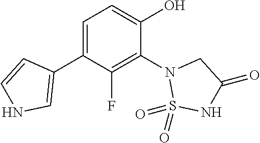 [SMILES: O=C1CN(c2c(O)ccc(-c3cc[nH]c3)c2F)S(=O)(=O)N1] | 5-(2-fluoro-6-hydroxy-3-(1H-pyrrol-3-yl)phenyl)- 1,2,5-thiadiazolidin-3-one 1,1-dioxide | 310.2 |
| 130 | 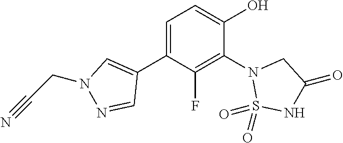 [SMILES: N#CCn1cc(-c2ccc(O)c(N3CC(=O)NS3(=O)=O)c2F)cn1] | 2-(4-(3-(1,1-dioxido-4-oxo-1,2,5-thiadiazolidin-2- yl)-2-fluoro-4-hydroxyphenyl)-1H-pyrazol-1- yl)acetonitrile | 350.1 |
| 131 | 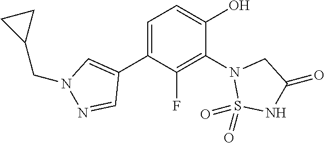 [SMILES: O=C1CN(c2c(O)ccc(-c3cnn(CC4CC4)c3)c2F)S(=O)(=O)N1] | 5-(3-(1-(cyclopropylmethyl)-1H-pyrazol-4-yl)-2- fluoro-6-hydroxyphenyl)-1,2,5-thiadiazolidin-3-one 1,1-dioxide | 365.0 |
| 132 | 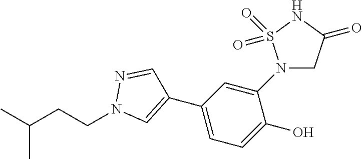 [SMILES: CC(C)CCn1cc(-c2ccc(O)c(N3CC(=O)NS3(=O)=O)c2)cn1] | 5-(2-hydroxy-5-(1-isopentyl-1H-pyrazol-4- yl)phenyl)-1,2,5-thiadiazolidin-3-one 1,1-dioxide | 363.2 |
| 133 | 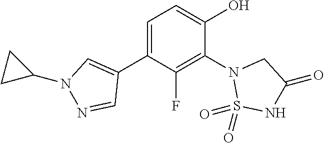 [SMILES: O=C1CN(c2c(O)ccc(-c3cnn(C4CC4)c3)c2F)S(=O)(=O)N1] | 5-(3-(1-cyclopropyl-1H-pyrazol-4-yl)-2-fluoro-6- hydroxyphenyl)-1,2,5-thiadiazolidin-3-one 1,1- dioxide | 351.1 |
| 134 | 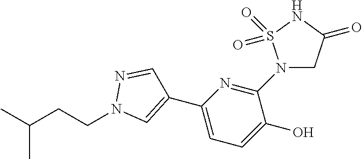 [SMILES: CC(C)CCn1cc(-c2ccc(O)c(N3CC(=O)NS3(=O)=O)n2)cn1] | 5-(3-hydroxy-6-(1-isopentyl-1H-pyrazol-4- yl)pyridin-2-yl)-1,2,5-thiadiazolidin-3-one 1,1- dioxide | 364.3 |
| 135 | 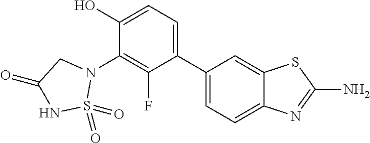 [SMILES: Nc1nc2ccc(-c3ccc(O)c(N4CC(=O)NS4(=O)=O)c3F)cc2s1] | 5-(3-(2-aminobenzo[d]thiazol-6-yl)-2-fluoro-6- hydroxyphenyl)-1,2,5-thiadiazolidin-3-one 1,1- dioxide | 393.0 |
| 136 | 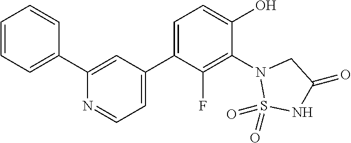 [SMILES: O=C1CN(c2c(O)ccc(-c3ccnc(-c4ccccc4)c3)c2F)S(=O)(=O)N1] | 5-(2-fluoro-6-hydroxy-3-(2-phenylpyridin-4- yl)phenyl)-1,2,5-thiadiazolidin-3-one 1,1-dioxide | 398.2 |
| 137 | 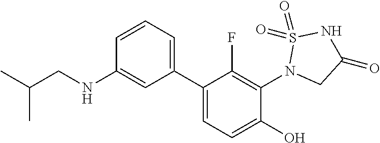 [SMILES: CC(C)CNc1cccc(-c2ccc(O)c(N3CC(=O)NS3(=O)=O)c2F)c1] | 5-(2-fluoro-4-hydroxy-3′-(isobutylamino)-[1,1′- biphenyl]-3-yl)-1,2,5-thiadiazolidin-3-one 1,1- dioxide | 392.0 |
| 138 | 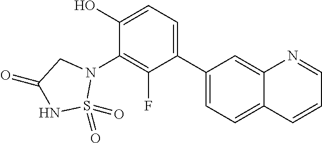 [SMILES: O=C1CN(c2c(O)ccc(-c3ccc4cccnc4c3)c2F)S(=O)(=O)N1] | 5-(2-fluoro-6-hydroxy-3-(quinolin-7-yl)phenyl)- 1,2,5-thiadiazolidin-3-one 1,1-dioxide | 372.0 |
| 139 | 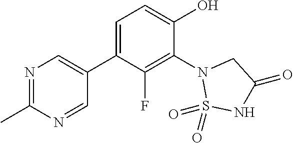 [SMILES: Cc1ncc(-c2ccc(O)c(N3CC(=O)NS3(=O)=O)c2F)cn1] | 5-(2-fluoro-6-hydroxy-3-(2-methylpyrimidin-5- yl)phenyl)-1,2,5-thiadiazolidin-3-one 1,1-dioxide | 337.0 |
| 140 | 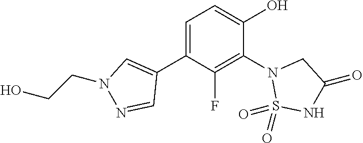 [SMILES: O=C1CN(c2c(O)ccc(-c3cnn(CCO)c3)c2F)S(=O)(=O)N1] | 5-(2-fluoro-6-hydroxy-3-(1-(2-hydroxyethyl)-1H- pyrazol-4-yl)phenyl)-1,2,5-thiadiazolidin-3-one 1,1- dioxide | 355.1 |
| 141 | 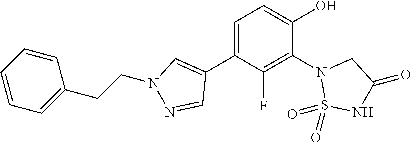 [SMILES: O=C1CN(c2c(O)ccc(-c3cnn(CCc4ccccc4)c3)c2F)S(=O)(=O)N1] | 5-(2-fluoro-6-hydroxy-3-(1-phenethyl-1H-1,2,3- triazol-4-yl)phenyl)-1,2,5-thiadiazolidin-3-one 1,1- dioxide | 416.1 |
| 142 | 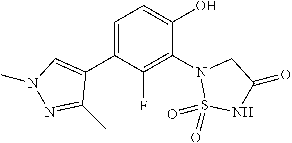 [SMILES: Cc1nn(C)cc1-c1ccc(O)c(N2CC(=O)NS2(=O)=O)c1F] | 5-(3-(1,3-dimethyl-1H-pyrazol-4-yl)-2-fluoro-6- hydroxyphenyl)-1,2,5-thiadiazolidin-3-one 1,1- dioxide | 339.2 |
| 143 | 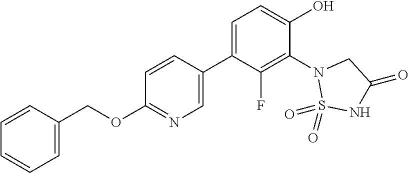 [SMILES: O=C1CN(c2c(O)ccc(-c3ccc(OCc4ccccc4)nc3)c2F)S(=O)(=O)N1] | 5-(3-(6-(benzyloxy)pyridin-3-yl)-2-fluoro-6- hydroxyphenyl)-1,2,5-thiadiazolidin-3-one 1,1- dioxide | 428.0 |
| 144 | 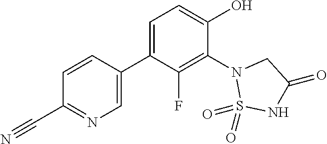 [SMILES: N#Cc1ccc(-c2ccc(O)c(N3CC(=O)NS3(=O)=O)c2F)cn1] | 5-(3-(1,1-dioxido-4-oxo-1,2,5-thiadiazolidin-2-yl)-2- fluoro-4-hydroxyphenyl)picolinonitrile | 347.0 |
| 145 | 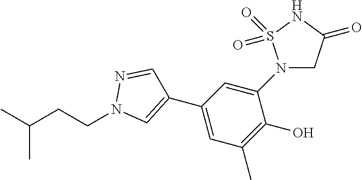 [SMILES: Cc1cc(-c2cnn(CCC(C)C)c2)cc(N2CC(=O)NS2(=O)=O)c1O] | 5-(2-hydroxy-5-(1-isopentyl-1H-pyrazol-4-yl)-3- methylphenyl)-1,2,5-thiadiazolidin-3-one 1,1-dioxide | 377.2 |
| 146 | 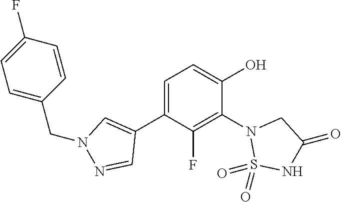 [SMILES: O=C1CN(c2c(O)ccc(-c3cnn(Cc4ccc(F)cc4)c3)c2F)S(=O)(=O)N1] | 5-(2-fluoro-3-(1-(4-fluorobenzyl)-1H-imidazol-4-yl)- 6-hydroxyphenyl)-1,2,5-thiadiazolidin-3-one 1,1- dioxide | 419.2 |
| 147 | 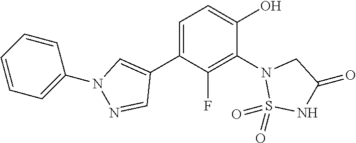 [SMILES: O=C1CN(c2c(O)ccc(-c3cnn(-c4ccccc4)c3)c2F)S(=O)(=O)N1] | 5-(2-fluoro-6-hydroxy-3-(1-phenyl-1H-pyrazol-4- yl)phenyl)-1,2,5-thiadiazolidin-3-one 1,1-dioxide | 387.0 |
| 148 | 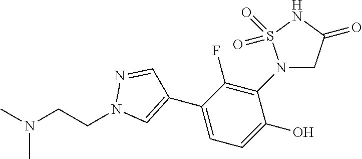 [SMILES: CN(C)CCn1cc(-c2ccc(O)c(N3CC(=O)NS3(=O)=O)c2F)cn1] | 5-(3-(1-(2-(dimethylamino)ethyl)-1H-pyrazol-4-yl)- 2-fluoro-6-hydroxyphenyl)-1,2,5-thiadiazolidin-3- one 1,1-dioxide | 382.0 |
| 149 | 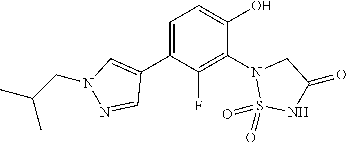 [SMILES: CC(C)Cn1cc(-c2ccc(O)c(N3CC(=O)NS3(=O)=O)c2F)cn1] | 5-(2-fluoro-6-hydroxy-3-(1-isobutyl-1H-pyrazol-4- yl)phenyl)-1,2,5-thiadiazolidin-3-one 1,1-dioxide | 366.9 |
| 150 | 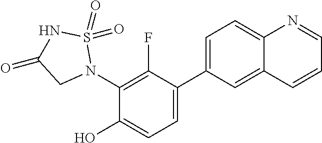 [SMILES: O=C1CN(c2c(O)ccc(-c3ccc4ncccc4c3)c2F)S(=O)(=O)N1] | 5-(2-fluoro-6-hydroxy-3-(quinolin-6-yl)phenyl)- 1,2,5-thiadiazolidin-3-one 1,1-dioxide | 372.3 |
| 151 | 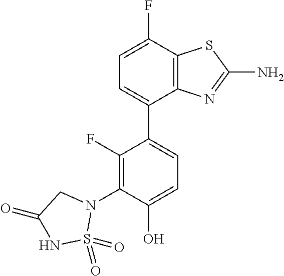 [SMILES: Nc1nc2c(-c3ccc(O)c(N4CC(=O)NS4(=O)=O)c3F)ccc(F)c2s1] | 5-(3-(2-amino-7-fluorobenzo[d]thiazol-4-yl)-2- fluoro-6-hydroxyphenyl)-1,2,5-thiadiazolidin-3-one 1,1-dioxide | 411.0 |
| 152 | 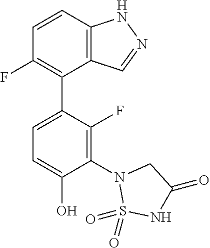 [SMILES: O=C1CN(c2c(O)ccc(-c3c(F)ccc4[nH]ncc34)c2F)S(=O)(=O)N1] | 5-(2-fluoro-3-(5-fluoro-1H-indazol-4-yl)-6- hydroxyphenyl)-1,2,5-thiadiazolidin-3-one 1,1- dioxide | 378.9 |
| 153 | 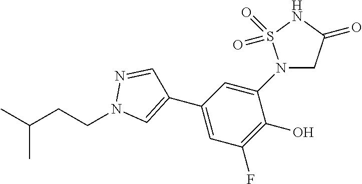 [SMILES: CC(C)CCn1cc(-c2cc(F)c(O)c(N3CC(=O)NS3(=O)=O)c2)cn1] | 5-(3-fluoro-2-hydroxy-5-(1-isopentyl-1H-pyrazol-4- yl)phenyl)-1,2,5-thiadiazolidin-3-one 1,1-dioxide | 381.1 |
| 154 | 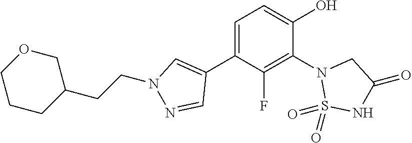 [SMILES: O=C1CN(c2c(O)ccc(-c3cnn(CCC4CCCOC4)c3)c2F)S(=O)(=O)N1] | 5-(2-fluoro-6-hydroxy-3-(1-(2-(tetrahydro-2H-pyran- 3-yl)ethyl)-1H-pyrazol-4-yl)phenyl)-1,2,5- thiadiazolidin-3-one 1,1-dioxide | 423.0 |
| 155 | 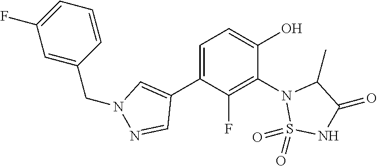 [SMILES: CC1C(=O)NS(=O)(=O)N1c1c(O)ccc(-c2cnn(Cc3cccc(F)c3)c2)c1F] | 5-(2-fluoro-3-(1-(3-fluorobenzyl)-1H-pyrazol-4-yl)- 6-hydroxyphenyl)-4-methyl-1,2,5-thiadiazolidin-3- one 1,1-dioxide | 433.0 |
| 156 | 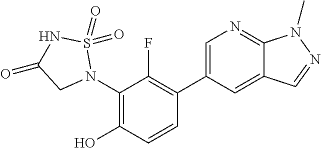 [SMILES: Cn1ncc2cc(-c3ccc(O)c(N4CC(=O)NS4(=O)=O)c3F)cnc21] | 5-(2-fluoro-6-hydroxy-3-(1-methyl-1H-pyrazolo[3,4- b]pyridin-5-yl)phenyl)-1,2,5-thiadiazolidin-3-one 1,1-dioxide | 376.0 |
| 157 | 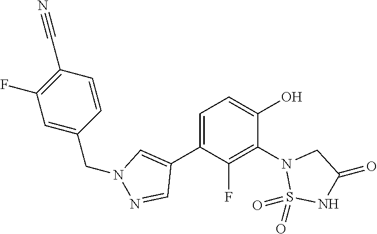 [SMILES: N#Cc1ccc(Cn2cc(-c3ccc(O)c(N4CC(=O)NS4(=O)=O)c3F)cn2)cc1F] | 4-((4-(3-(1,1-dioxido-4-oxo-1,2,5-thiadiazolidin-2- yl)-2-fluoro-4-hydroxyphenyl)-1H-pyrazol-1- yl)methyl)-2-fluorobenzonitrile | 444.0 |
| 158 | 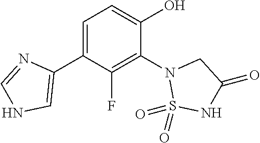 [SMILES: O=C1CN(c2c(O)ccc(-c3c[nH]cn3)c2F)S(=O)(=O)N1] | 5-(2-fluoro-6-hydroxy-3-(1H-imidazol-4-yl)phenyl)- 1,2,5-thiadiazolidin-3-one 1,1-dioxide | 310.9 |
| 159 | 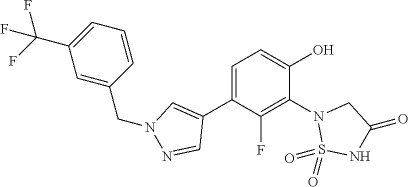 [SMILES: O=C1CN(c2c(O)ccc(-c3cnn(Cc4cccc(C(F)(F)F)c4)c3)c2F)S(=O)(=O)N1] | 5-(2-fluoro-6-hydroxy-3-(1-(3- (trifluoromethyl)benzyl)-1H-pyrazol-4-yl)phenyl)- 1,2,5-thiadiazolidin-3-one 1,1-dioxide | 469.1 |
| 160 | 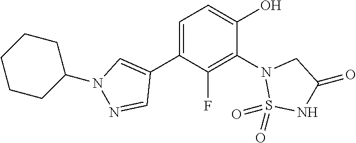 [SMILES: O=C1CN(c2c(O)ccc(-c3cnn(C4CCCCC4)c3)c2F)S(=O)(=O)N1] | 5-(3-(1-cyclohexyl-1H-pyrazol-4-yl)-2-fluoro-6- hydroxyphenyl)-1,2,5-thiadiazolidin-3-one 1,1- dioxide | 392.9 |
| 161 | 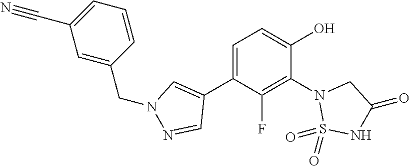 [SMILES: N#Cc1cccc(Cn2cc(-c3ccc(O)c(N4CC(=O)NS4(=O)=O)c3F)cn2)c1] | 3-((4-(3-(1,1-dioxido-4-oxo-1,2,5-thiadiazolidin-2- yl)-2-fluoro-4-hydroxyphenyl)-1H-pyrazol-1- yl)methyl)benzonitrile | 426.0 |
| 162 | 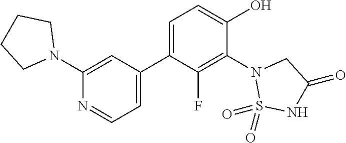 [SMILES: O=C1CN(c2c(O)ccc(-c3ccnc(N4CCCC4)c3)c2F)S(=O)(=O)N1] | 5-(2-fluoro-6-hydroxy-3-(2-(pyrrolidin-1-yl)pyridin- 4-yl)phenyl)-1,2,5-thiadiazolidin-3-one 1,1-dioxide | 391.2 |
| 163 | 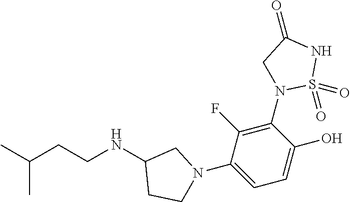 [SMILES: CC(C)CCNC1CCN(c2ccc(O)c(N3CC(=O)NS3(=O)=O)c2F)C1] | 5-(2-fluoro-6-hydroxy-3-(3- (isopentylamino)pyrrolidin-1-yl)phenyl)-1,2,5- thiadiazolidin-3-one 1,1-dioxide | 399.1 |
| 164 | 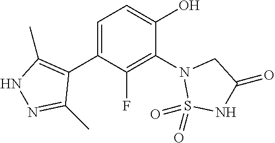 [SMILES: Cc1n[nH]c(C)c1-c1ccc(O)c(N2CC(=O)NS2(=O)=O)c1F] | 5-(3-(3,5-dimethyl-1H-pyrazol-4-yl)-2-fluoro-6- hydroxyphenyl)-1,2,5-thiadiazolidin-3-one 1,1- dioxide | 339.1 |
| 165 | 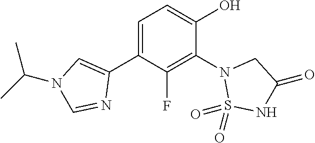 [SMILES: CC(C)n1cnc(-c2ccc(O)c(N3CC(=O)NS3(=O)=O)c2F)c1] | 5-(2-fluoro-6-hydroxy-3-(1-isopropyl-1H-imidazol- 4-yl)phenyl)-1,2,5-thiadiazolidin-3-one 1,1-dioxide | 353.2 |
| 166 | 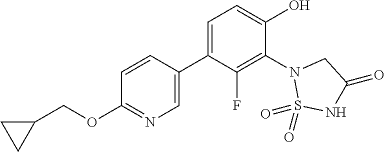 [SMILES: O=C1CN(c2c(O)ccc(-c3ccc(OCC4CC4)nc3)c2F)S(=O)(=O)N1] | 5-(3-(6-(cyclopropylmethoxy)pyridin-3-yl)-2-fluoro- 6-hydroxyphenyl)-1,2,5-thiadiazolidin-3-one 1,1- dioxide | 392.0 |
| 167 | 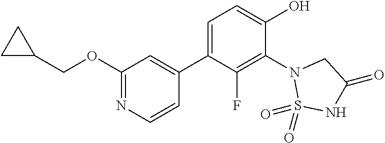 [SMILES: O=C1CN(c2c(O)ccc(-c3ccnc(OCC4CC4)c3)c2F)S(=O)(=O)N1] | 5-(3-(2-(cyclopropylmethoxy)pyridin-4-yl)-2-fluoro- 6-hydroxyphenyl)-1,2,5-thiadiazolidin-3-one 1,1- dioxide | 392.1 |
| 168 | 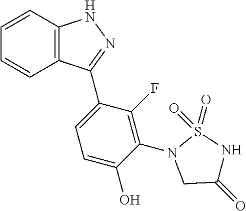 [SMILES: O=C1CN(c2c(O)ccc(-c3n[nH]c4ccccc34)c2F)S(=O)(=O)N1] | 5-(2-fluoro-6-hydroxy-3-(1H-indazol-3-yl)phenyl)- 1,2,5-thiadiazolidin-3-one 1,1-dioxide | 361.1 |
| 169 | 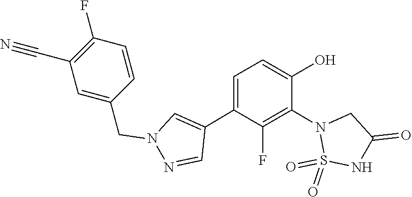 [SMILES: N#Cc1cc(Cn2cc(-c3ccc(O)c(N4CC(=O)NS4(=O)=O)c3F)cn2)ccc1F] | 5-((4-(3-(1,1-dioxido-4-oxo-1,2,5-thiadiazolidin-2- yl)-2-fluoro-4-hydroxyphenyl)-1H-pyrazol-1- yl)methyl)-2-fluorobenzonitrile | 444.0 |
| 170 | 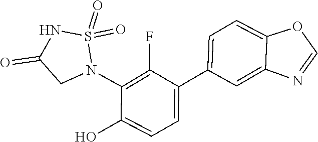 [SMILES: O=C1CN(c2c(O)ccc(-c3ccc4ocnc4c3)c2F)S(=O)(=O)N1] | 5-(3-(benzo[d]oxazol-5-yl)-2-fluoro-6- hydroxyphenyl)-1,2,5-thiadiazolidin-3-one 1,1- dioxide | 361.9 |
| 171 | 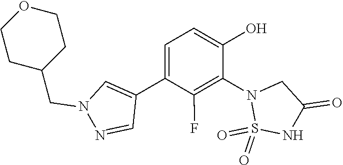 [SMILES: O=C1CN(c2c(O)ccc(-c3cnn(CC4CCOCC4)c3)c2F)S(=O)(=O)N1] | 5-(2-fluoro-6-hydroxy-3-(1-((tetrahydro-2H-pyran-4- yl)methyl)-1H-pyrazol-4-yl)phenyl)-1,2,5- thiadiazolidin-3-one 1,1-dioxide | 409.2 |
| 172 | 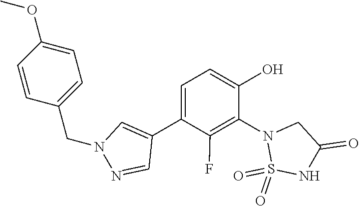 [SMILES: COc1ccc(Cn2cc(-c3ccc(O)c(N4CC(=O)NS4(=O)=O)c3F)cn2)cc1] | 5-(2-fluoro-6-hydroxy-3-(1-(4-methoxybenzyl)-1H- pyrazol-4-yl)phenyl)-1,2,5-thiadiazolidin-3-one 1,1- dioxide | 431.1 |
| 173 | 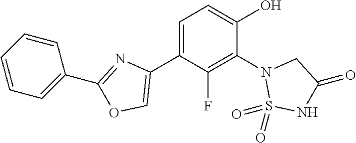 [SMILES: O=C1CN(c2c(O)ccc(-c3coc(-c4ccccc4)n3)c2F)S(=O)(=O)N1] | 5-(2-fluoro-6-hydroxy-3-(2-phenyloxazol-4- yl)phenyl)-1,2,5-thiadiazolidin-3-one 1,1-dioxide | 388.0 |
| 174 | 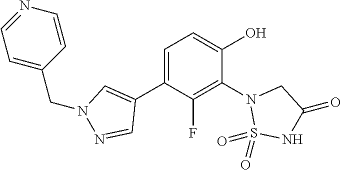 [SMILES: O=C1CN(c2c(O)ccc(-c3cnn(Cc4ccncc4)c3)c2F)S(=O)(=O)N1] | 5-(2-fluoro-6-hydroxy-3-(1-(pyridin-4-ylmethyl)-1H- pyrazol-4-yl)phenyl)-1,2,5-thiadiazolidin-3-one 1,1- dioxide | 404.0 [M + H]⁺ |
| 175 | 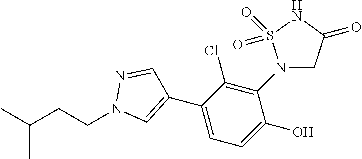 [SMILES: CC(C)CCn1cc(-c2ccc(O)c(N3CC(=O)NS3(=O)=O)c2Cl)cn1] | 5-(2-chloro-6-hydroxy-3-(1-isopentyl-1H-pyrazol-4- yl)phenyl)-1,2,5-thiadiazolidin-3-one 1,1-dioxide | 397.1 |
| 176 | 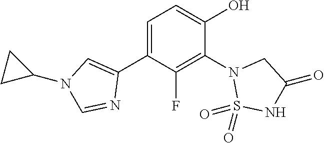 [SMILES: O=C1CN(c2c(O)ccc(-c3cn(C4CC4)cn3)c2F)S(=O)(=O)N1] | 5-(3-(1-cyclopropyl-1H-imidazol-4-yl)-2-fluoro-6- hydroxyphenyl)-1,2,5-thiadiazolidin-3-one 1,1- dioxide | 351.0 |
| 177 | 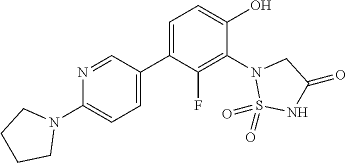 [SMILES: O=C1CN(c2c(O)ccc(-c3ccc(N4CCCC4)nc3)c2F)S(=O)(=O)N1] | 5-(2-fluoro-6-hydroxy-3-(6-(pyrrolidin-1-yl)pyridin- 3-yl)phenyl)-1,2,5-thiadiazolidin-3-one 1,1-dioxide | 391.0 |
| 178 | 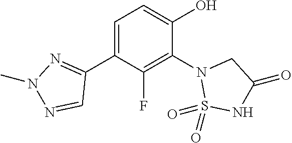 [SMILES: Cn1ncc(-c2ccc(O)c(N3CC(=O)NS3(=O)=O)c2F)n1] | 5-(2-fluoro-6-hydroxy-3-(2-methyl-2H-1,2,3-triazol- 4-yl)phenyl)-1,2,5-thiadiazolidin-3-one 1,1-dioxide | 325.9 |
| 179 | 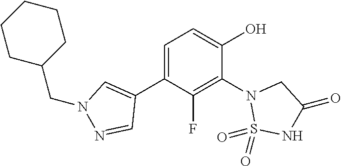 [SMILES: O=C1CN(c2c(O)ccc(-c3cnn(CC4CCCCC4)c3)c2F)S(=O)(=O)N1] | 5-(3-(1-(cyclohexylmethyl)-1H-pyrazol-4-yl)-2- fluoro-6-hydroxyphenyl)-1,2,5-thiadiazolidin-3-one 1,1-dioxide | 407.3 |
| 180 | 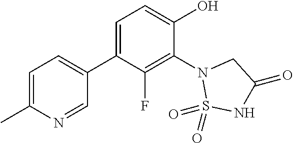 [SMILES: Cc1ccc(-c2ccc(O)c(N3CC(=O)NS3(=O)=O)c2F)cn1] | 5-(2-fluoro-6-hydroxy-3-(6-methylpyridin-3- yl)phenyl)-1,2,5-thiadiazolidin-3-one 1,1-dioxide | 336.1 |
| 181 | 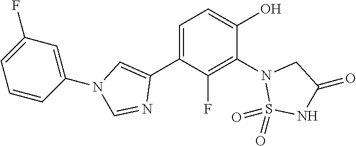 [SMILES: O=C1CN(c2c(O)ccc(-c3cn(-c4cccc(F)c4)cn3)c2F)S(=O)(=O)N1] | 5-(2-fluoro-3-(1-(3-fluorophenyl)-1H-imidazol-4-yl)- 6-hydroxyphenyl)-1,2,5-thiadiazolidin-3-one 1,1- dioxide | 405.1 |
| 182 | 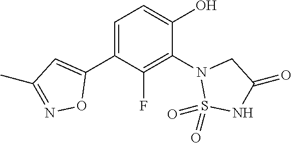 [SMILES: Cc1cc(-c2ccc(O)c(N3CC(=O)NS3(=O)=O)c2F)on1] | 5-(2-fluoro-6-hydroxy-3-(3-methylisoxazol-5- yl)phenyl)-1,2,5-thiadiazolidin-3-one 1,1-dioxide | 326.1 |
| 183 | 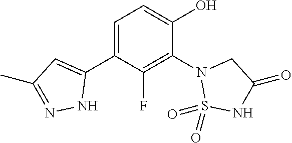 [SMILES: Cc1cc(-c2ccc(O)c(N3CC(=O)NS3(=O)=O)c2F)[nH]n1] | 5-(2-fluoro-6-hydroxy-3-(3-methyl-1H-pyrazol-5- yl)phenyl)-1,2,5-thiadiazolidin-3-one 1,1-dioxide | 325.1 |
| 184 | 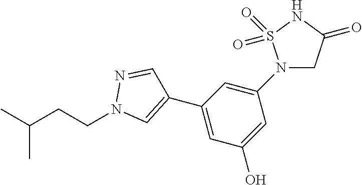 [SMILES: CC(C)CCn1cc(-c2cc(O)cc(N3CC(=O)NS3(=O)=O)c2)cn1] | 5-(3-hydroxy-5-(1-isopentyl-1H-pyrazol-4- yl)phenyl)-1,2,5-thiadiazolidin-3-one 1,1-dioxide | 363.2 |
| 185 | 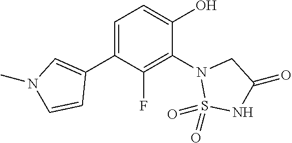 [SMILES: Cn1ccc(-c2ccc(O)c(N3CC(=O)NS3(=O)=O)c2F)c1] | 5-(2-fluoro-6-hydroxy-3-(1-methyl-1H-pyrrol-3- yl)phenyl)-1,2,5-thiadiazolidin-3-one 1,1-dioxide | 323.9 |
| 186 | 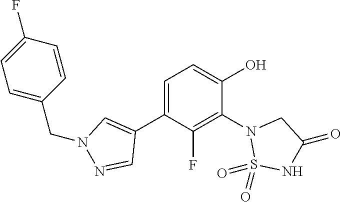 [SMILES: O=C1CN(c2c(O)ccc(-c3cnn(Cc4ccc(F)cc4)c3)c2F)S(=O)(=O)N1] | 5-(2-fluoro-3-(1-(4-fluorobenzyl)-1H-pyrazol-4-yl)- 6-hydroxyphenyl)-1,2,5-thiadiazolidin-3-one 1,1- dioxide | 419.2 |
| 187 | 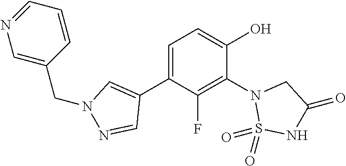 [SMILES: O=C1CN(c2c(O)ccc(-c3cnn(Cc4cccnc4)c3)c2F)S(=O)(=O)N1] | 5-(2-fluoro-6-hydroxy-3-(1-(pyridin-3-ylmethyl)-1H- pyrazol-4-yl)phenyl)-1,2,5-thiadiazolidin-3-one 1,1- dioxide | 402.1 |
| 188 | 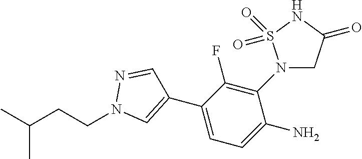 [SMILES: CC(C)CCn1cc(-c2ccc(N)c(N3CC(=O)NS3(=O)=O)c2F)cn1] | 5-(6-amino-2-fluoro-3-(1-isopentyl-1H-pyrazol-4- yl)phenyl)-1,2,5-thiadiazolidin-3-one 1,1-dioxide | 380.2 |
| 189 | 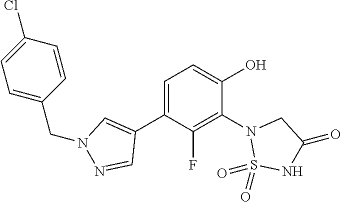 [SMILES: O=C1CN(c2c(O)ccc(-c3cnn(Cc4ccc(Cl)cc4)c3)c2F)S(=O)(=O)N1] | 5-(3-(1-(4-chlorobenzyl)-1H-pyrazol-4-yl)-2-fluoro- 6-hydroxyphenyl)-1,2,5-thiadiazolidin-3-one 1,1- dioxide | 435.0 |
| 190 | 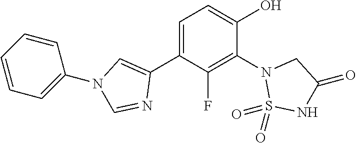 [SMILES: O=C1CN(c2c(O)ccc(-c3cn(-c4ccccc4)cn3)c2F)S(=O)(=O)N1] | 5-(2-fluoro-6-hydroxy-3-(1-phenyl-1H-imidazol-4- yl)phenyl)-1,2,5-thiadiazolidin-3-one 1,1-dioxide | 387.0 |
| 191 | 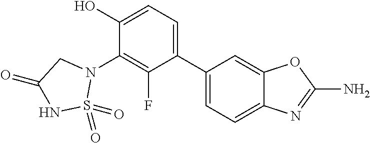 [SMILES: Nc1nc2ccc(-c3ccc(O)c(N4CC(=O)NS4(=O)=O)c3F)cc2o1] | 5-(3-(2-aminobenzo[d]oxazol-6-yl)-2-fluoro-6- hydroxyphenyl)-1,2,5-thiadiazolidin-3-one 1,1- dioxide | 377.0 |
| 192 | 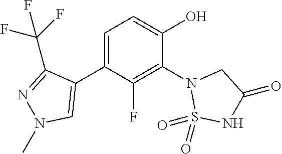 [SMILES: Cn1cc(-c2ccc(O)c(N3CC(=O)NS3(=O)=O)c2F)c(C(F)(F)F)n1] | 5-(2-fluoro-6-hydroxy-3-(1-methyl-3- (trifluoromethyl)-1H-pyrazol-4-yl)phenyl)-1,2,5- thiadiazolidin-3-one 1,1-dioxide | 393.1 |
| 193 | 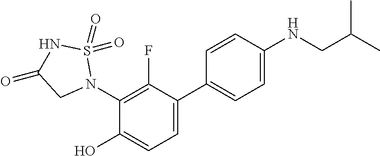 [SMILES: CC(C)CNc1ccc(-c2ccc(O)c(N3CC(=O)NS3(=O)=O)c2F)cc1] | 5-(2-fluoro-4-hydroxy-4′-(isobutylamino)-[1,1′- biphenyl]-3-yl)-1,2,5-thiadiazolidin-3-one 1,1- dioxide | 392.0 |
| 194 | 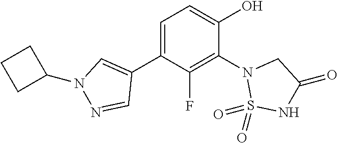 [SMILES: O=C1CN(c2c(O)ccc(-c3cnn(C4CCC4)c3)c2F)S(=O)(=O)N1] | 5-(3-(1-cyclobutyl-1H-pyrazol-4-yl)-2-fluoro-6- hydroxyphenyl)-1,2,5-thiadiazolidin-3-one 1,1- dioxide | 365.0 |
| 195 | 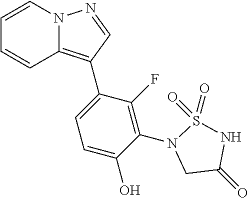 [SMILES: O=C1CN(c2c(O)ccc(-c3cnn4ccccc34)c2F)S(=O)(=O)N1] | 5-(2-fluoro-6-hydroxy-3-(pyrazolo[1,5-a]pyridin-3- yl)phenyl)-1,2,5-thiadiazolidin-3-one 1,1-dioxide | 361.2 |
| 196 | 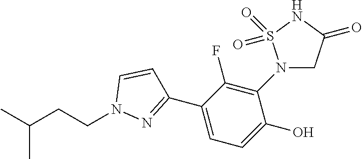 [SMILES: CC(C)CCn1ccc(-c2ccc(O)c(N3CC(=O)NS3(=O)=O)c2F)n1] | 5-(2-fluoro-6-hydroxy-3-(1-isopentyl-1H-pyrazol-3- yl)phenyl)-1,2,5-thiadiazolidin-3-one 1,1-dioxide | 381.0 |
| 197 | 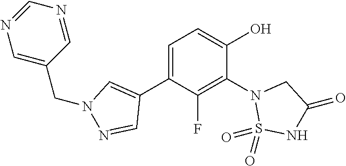 [SMILES: O=C1CN(c2c(O)ccc(-c3cnn(Cc4cncnc4)c3)c2F)S(=O)(=O)N1] | 5-(2-fluoro-6-hydroxy-3-(1-(pyrimidin-5-ylmethyl)- 1H-pyrazol-4-yl)phenyl)-1,2,5-thiadiazolidin-3-one 1,1-dioxide | 403.1 |
| 198 | 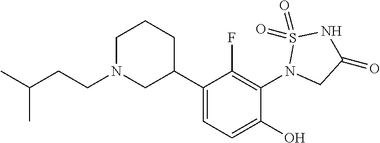 [SMILES: CC(C)CCN1CCCC(c2ccc(O)c(N3CC(=O)NS3(=O)=O)c2F)C1] | 5-(2-fluoro-6-hydroxy-3-(1-isopentylpiperidin-3- yl)phenyl)-1,2,5-thiadiazolidin-3-one 1,1-dioxide | 398.3 |
| 199 | 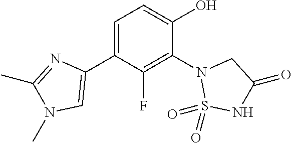 [SMILES: Cc1nc(-c2ccc(O)c(N3CC(=O)NS3(=O)=O)c2F)cn1C] | 5-(3-(1,2-dimethyl-1H-imidazol-4-yl)-2-fluoro-6- hydroxyphenyl)-1,2,5-thiadiazolidin-3-one 1,1- dioxide | 339.0 |
| 200 | 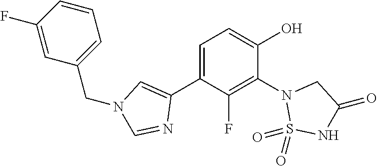 [SMILES: O=C1CN(c2c(O)ccc(-c3cn(Cc4cccc(F)c4)cn3)c2F)S(=O)(=O)N1] | 5-(2-fluoro-3-(1-(3-fluorobenzyl)-1H-imidazol-4-yl)- 6-hydroxyphenyl)-1,2,5-thiadiazolidin-3-one 1,1- dioxide | 419.1 |
| 201 | 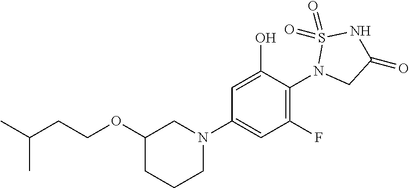 [SMILES: CC(C)CCOC1CCCN(c2cc(O)c(N3CC(=O)NS3(=O)=O)c(F)c2)C1] | 5-(2-fluoro-6-hydroxy-4-(3-(isopentyloxy)piperidin- 1-yl)phenyl)-1,2,5-thiadiazolidin-3-one 1,1-dioxide | 414.1 |
| 202 | 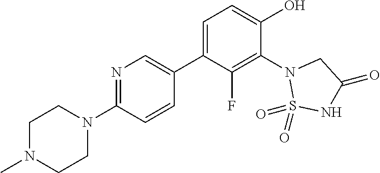 [SMILES: CN1CCN(c2ccc(-c3ccc(O)c(N4CC(=O)NS4(=O)=O)c3F)cn2)CC1] | 5-(2-fluoro-6-hydroxy-3-(6-(4-methylpiperazin-1- yl)pyridin-3-yl)phenyl)-1,2,5-thiadiazolidin-3-one 1,1-dioxide | 420.1 |
| 203 | 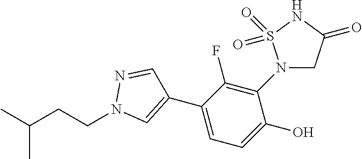 [SMILES: CC(C)CCn1cc(-c2ccc(O)c(N3CC(=O)NS3(=O)=O)c2F)cn1] | 5-(2-fluoro-6-hydroxy-3-(1-isopentyl-1H-pyrazol-4- yl)phenyl)-1,2,5-thiadiazolidin-3-one 1,1-dioxide | 381.1 |
| 204 | 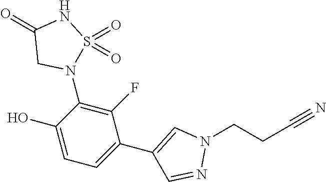 [SMILES: N#CCCn1cc(-c2ccc(O)c(N3CC(=O)NS3(=O)=O)c2F)cn1] | 3-(4-(3-(1,1-dioxido-4-oxo-1,2,5-thiadiazolidin-2- yl)-2-fluoro-4-hydroxyphenyl)-1H-pyrazol-1- yl)propanenitrile | 364.0 |
| 205 | 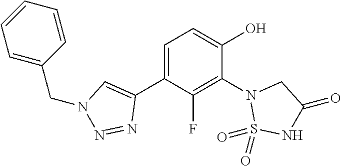 [SMILES: O=C1CN(c2c(O)ccc(-c3cn(Cc4ccccc4)nn3)c2F)S(=O)(=O)N1] | 5-(3-(1-benzyl-1H-1,2,3-triazol-4-yl)-2-fluoro-6- hydroxyphenyl)-1,2,5-thiadiazolidin-3-one 1,1- dioxide | 402.1 |
| 206 | 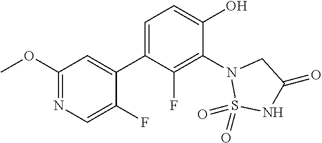 [SMILES: COc1cc(-c2ccc(O)c(N3CC(=O)NS3(=O)=O)c2F)c(F)cn1] | 5-(2-fluoro-3-(5-fluoro-2-methoxypyridin-4-yl)-6- hydroxyphenyl)-1,2,5-thiadiazolidin-3-one 1,1- dioxide | 370.1 |
| 207 | 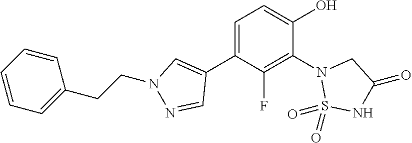 [SMILES: O=C1CN(c2c(O)ccc(-c3cnn(CCc4ccccc4)c3)c2F)S(=O)(=O)N1] | 5-(2-fluoro-6-hydroxy-3-(1-phenethyl-1H-pyrazol-4- yl)phenyl)-1,2,5-thiadiazolidin-3-one 1,1-dioxide | 415.0 |
| 208 | 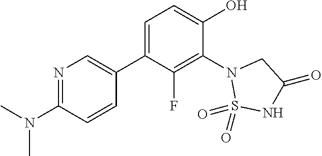 [SMILES: CN(C)c1ccc(-c2ccc(O)c(N3CC(=O)NS3(=O)=O)c2F)cn1] | 5-(3-(6-(dimethylamino)pyridin-3-yl)-2-fluoro-6- hydroxyphenyl)-1,2,5-thiadiazolidin-3-one 1,1- dioxide | 365.1 |
| 209 | 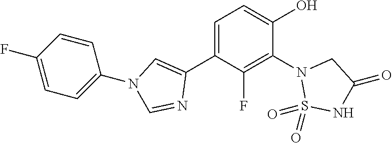 [SMILES: O=C1CN(c2c(O)ccc(-c3cn(-c4ccc(F)cc4)cn3)c2F)S(=O)(=O)N1] | 5-(2-fluoro-3-(1-(4-fluorophenyl)-1H-imidazol-4-yl)- 6-hydroxyphenyl)-1,2,5-thiadiazolidin-3-one 1,1- dioxide | 405.1 |
| 210 | 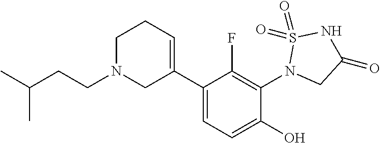 [SMILES: CC(C)CCN1CCC=C(c2ccc(O)c(N3CC(=O)NS3(=O)=O)c2F)C1] | 5-(2-fluoro-6-hydroxy-3-(1-isopentyl-1,2,5,6- tetrahydropyridin-3-yl)phenyl)-1,2,5-thiadiazolidin- 3-one 1,1-dioxide | 396.2 |
| 211 | 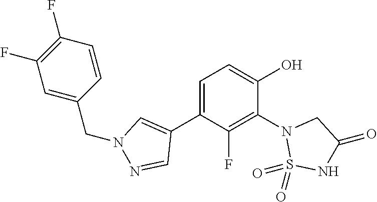 [SMILES: O=C1CN(c2c(O)ccc(-c3cnn(Cc4ccc(F)c(F)c4)c3)c2F)S(=O)(=O)N1] | 5-(3-(1-(3,4-difluorobenzyl)-1H-pyrazol-4-yl)-2- fluoro-6-hydroxyphenyl)-1,2,5-thiadiazolidin-3-one 1,1-dioxide | 437.2 |
| 212 | 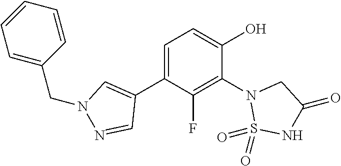 [SMILES: O=C1CN(c2c(O)ccc(-c3cnn(Cc4ccccc4)c3)c2F)S(=O)(=O)N1] | 5-(3-(1-benzyl-1H-pyrazol-4-yl)-2-fluoro-6- hydroxyphenyl)-1,2,5-thiadiazolidin-3-one 1,1- dioxide | 401.2 |
| 213 | 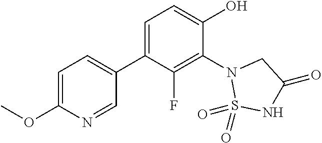 [SMILES: COc1ccc(-c2ccc(O)c(N3CC(=O)NS3(=O)=O)c2F)cn1] | 5-(2-fluoro-6-hydroxy-3-(6-methoxypyridin-3- yl)phenyl)-1,2,5-thiadiazolidin-3-one 1,1-dioxide | 352.1 |
| 214 | 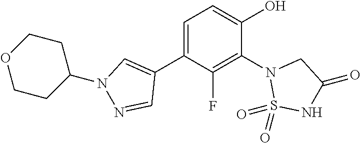 [SMILES: O=C1CN(c2c(O)ccc(-c3cnn(C4CCOCC4)c3)c2F)S(=O)(=O)N1] | 5-(2-fluoro-6-hydroxy-3-(1-(tetrahydro-2H-pyran-4- yl)-1H-pyrazol-4-yl)phenyl)-1,2,5-thiadiazolidin-3- one 1,1-dioxide | 397.1 [M + H]⁺ |
| 215 | 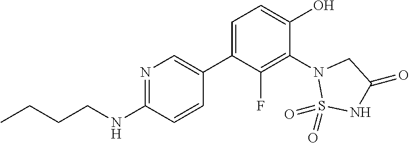 [SMILES: CCCCNc1ccc(-c2ccc(O)c(N3CC(=O)NS3(=O)=O)c2F)cn1] | 5-(3-(6-(butylamino)pyridin-3-yl)-2-fluoro-6- hydroxyphenyl)-1,2,5-thiadiazolidin-3-one 1,1- dioxide | 393.2 |
| 216 | 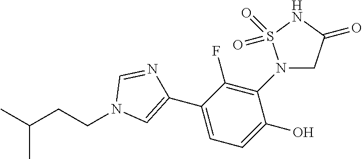 [SMILES: CC(C)CCn1cnc(-c2ccc(O)c(N3CC(=O)NS3(=O)=O)c2F)c1] | 5-(2-fluoro-6-hydroxy-3-(1-isopentyl-1H-imidazol- 4-yl)phenyl)-1,2,5-thiadiazolidin-3-one 1,1-dioxide | 381.1 [M + H]⁺ |
| 217 | 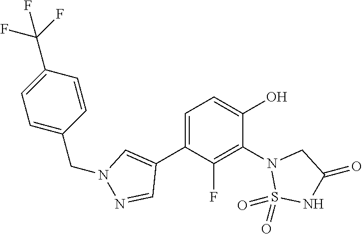 [SMILES: O=C1CN(c2c(O)ccc(-c3cnn(Cc4ccc(C(F)(F)F)cc4)c3)c2F)S(=O)(=O)N1] | 5-(2-fluoro-6-hydroxy-3-(1-(4- (trifluoromethyl)benzyl)-1H-pyrazol-4-yl)phenyl)- 1,2,5-thiadiazolidin-3-one 1,1-dioxide | 469.1 |
| 218 | 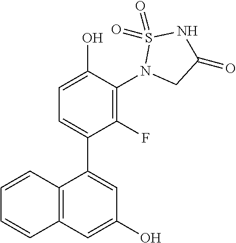 [SMILES: O=C1CN(c2c(O)ccc(-c3cc(O)cc4ccccc34)c2F)S(=O)(=O)N1] | 5-(2-fluoro-6-hydroxy-3-(3-hydroxynaphthalen-1- yl)phenyl)-1,2,5-thiadiazolidin-3-one 1,1-dioxide | 387.1 |
| 219 | 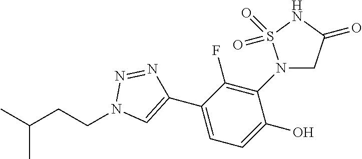 [SMILES: CC(C)CCn1cc(-c2ccc(O)c(N3CC(=O)NS3(=O)=O)c2F)nn1] | 5-(2-fluoro-6-hydroxy-3-(1-isopentyl-1H-1,2,3- triazol-4-yl)phenyl)-1,2,5-thiadiazolidin-3-one 1,1- dioxide | 382.1 |
| 220 | 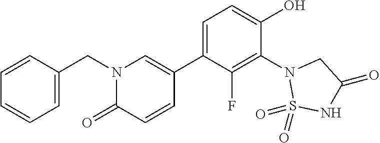 [SMILES: O=C1CN(c2c(O)ccc(-c3ccc(=O)n(Cc4ccccc4)c3)c2F)S(=O)(=O)N1] | 5-(3-(1-benzyl-6-oxo-1,6-dihydropyridin-3-yl)-2- fluoro-6-hydroxyphenyl)-1,2,5-thiadiazolidin-3-one 1,1-dioxide | 428.0 |
| 221 | 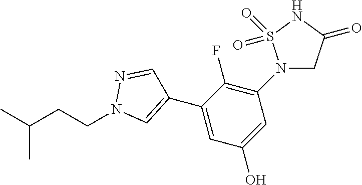 [SMILES: CC(C)CCn1cc(-c2cc(O)cc(N3CC(=O)NS3(=O)=O)c2F)cn1] | 5-(2-fluoro-5-hydroxy-3-(1-isopentyl-1H-pyrazol-4- yl)phenyl)-1,2,5-thiadiazolidin-3-one 1,1-dioxide | 381.1 |
| 222 | 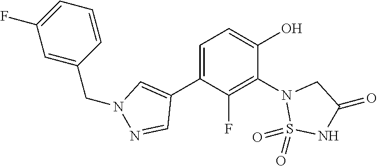 [SMILES: O=C1CN(c2c(O)ccc(-c3cnn(Cc4cccc(F)c4)c3)c2F)S(=O)(=O)N1] | 5-(2-fluoro-3-(1-(3-fluorobenzyl)-1H-pyrazol-4-yl)- 6-hydroxyphenyl)-1,2,5-thiadiazolidin-3-one 1,1- dioxide | 419.0 |
| 223 | 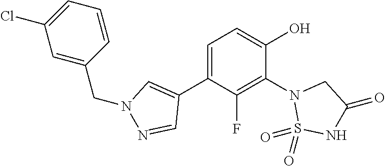 [SMILES: O=C1CN(c2c(O)ccc(-c3cnn(Cc4cccc(Cl)c4)c3)c2F)S(=O)(=O)N1] | 5-(3-(1-(3-chlorobenzyl)-1H-pyrazol-4-yl)-2-fluoro- 6-hydroxyphenyl)-1,2,5-thiadiazolidin-3-one 1,1- dioxide | 435.0 |
| 224 | 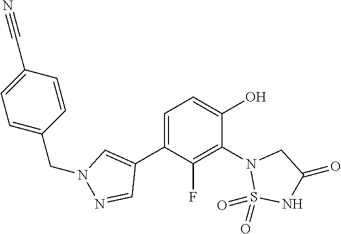 [SMILES: N#Cc1ccc(Cn2cc(-c3ccc(O)c(N4CC(=O)NS4(=O)=O)c3F)cn2)cc1] | 4-((4-(3-(1,1-dioxido-4-oxo-1,2,5-thiadiazolidin-2- yl)-2-fluoro-4-hydroxyphenyl)-1H-pyrazol-1- yl)methyl)benzonitrile | 426.2 |
| 225 | 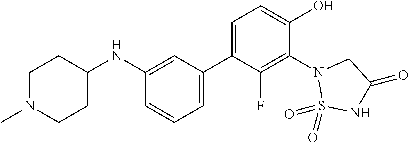 [SMILES: CN1CCC(Nc2cccc(-c3ccc(O)c(N4CC(=O)NS4(=O)=O)c3F)c2)CC1] | 5-(2-fluoro-4-hydroxy-3′-((1-methylpiperidin-4- yl)amino)-[1,1′-biphenyl]-3-yl)-1,2,5-thiadiazolidin- 3-one 1,1-dioxide | 433.0 |
| 226 | 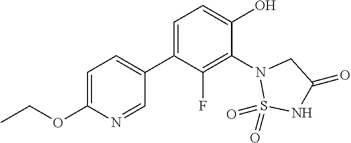 [SMILES: CCOc1ccc(-c2ccc(O)c(N3CC(=O)NS3(=O)=O)c2F)cn1] | 5-(3-(6-ethoxypyridin-3-yl)-2-fluoro-6- hydroxyphenyl)-1,2,5-thiadiazolidin-3-one 1,1- dioxide | 366.1 |
| 227 | 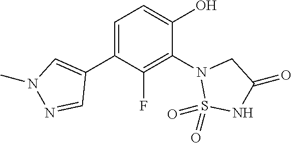 [SMILES: Cn1cc(-c2ccc(O)c(N3CC(=O)NS3(=O)=O)c2F)cn1] | 5-(2-fluoro-6-hydroxy-3-(1-methyl-1H-pyrazol-4- yl)phenyl)-1,2,5-thiadiazolidin-3-one 1,1-dioxide | 327.0 [M + H]⁺ |
| 228 | 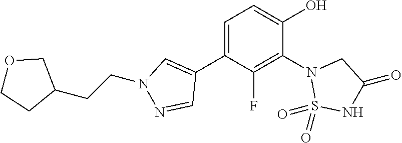 [SMILES: O=C1CN(c2c(O)ccc(-c3cnn(CCC4CCOC4)c3)c2F)S(=O)(=O)N1] | 5-(2-fluoro-6-hydroxy-3-(1-(2-(tetrahydrofuran-3- yl)ethyl)-1H-pyrazol-4-yl)phenyl)-1,2,5- thiadiazolidin-3-one 1,1-dioxide | 411.1 [M + H]⁺ |
| 229 | 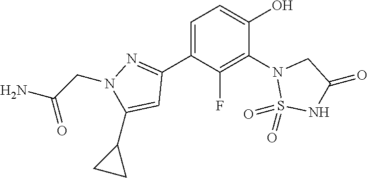 [SMILES: NC(=O)Cn1nc(-c2ccc(O)c(N3CC(=O)NS3(=O)=O)c2F)cc1C1CC1] | 2-(5-cyclopropyl-3-(3-(1,1-dioxido-4-oxo-1,2,5- thiadiazolidin-2-yl)-2-fluoro-4-hydroxyphenyl)-1H- pyrazol-1-yl)acetamide | 408.2 |
| 230 | 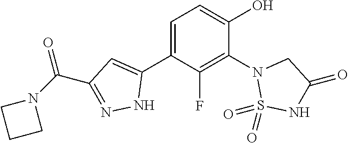 [SMILES: O=C1CN(c2c(O)ccc(-c3cc(C(=O)N4CCC4)n[nH]3)c2F)S(=O)(=O)N1] | 5-(3-(3-(azetidine-1-carbonyl)-1H-pyrazol-5-yl)-2- fluoro-6-hydroxyphenyl)-1,2,5-thiadiazolidin-3-one 1,1-dioxide | 394.2 |
| 231 | 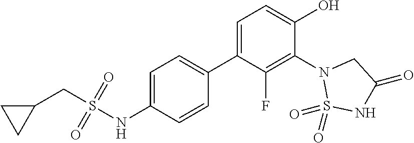 [SMILES: O=C1CN(c2c(O)ccc(-c3ccc(NS(=O)(=O)CC4CC4)cc3)c2F)S(=O)(=O)N1] | 1-cyclopropyl-N-(3′-(1,1-dioxido-4-oxo-1,2,5- thiadiazolidin-2-yl)-2′-fluoro-4′-hydroxy-[1,1′- biphenyl]-4-yl)methanesulfonamide | 454.1 |
| 232 | 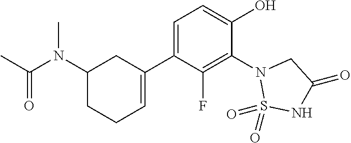 [SMILES: CC(=O)N(C)C1CCC=C(c2ccc(O)c(N3CC(=O)NS3(=O)=O)c2F)C1] | N-(3′-(1,1-dioxido-4-oxo-1,2,5-thiadiazolidin-2-yl)- 2′-fluoro-4′-hydroxy-2,3,4,5-tetrahydro-[1,1′- biphenyl]-3-yl)-N-methylacetamide | 396.2 |
| 233 | 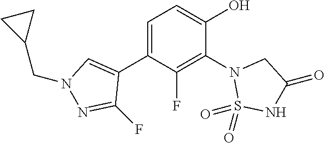 [SMILES: O=C1CN(c2c(O)ccc(-c3cn(CC4CC4)nc3F)c2F)S(=O)(=O)N1] | 5-(3-(1-(cyclopropylmethyl)-3-fluoro-1H-pyrazol-4- yl)-2-fluoro-6-hydroxyphenyl)-1,2,5-thiadiazolidin- 3-one 1,1-dioxide | 383.0 |
| 234 | 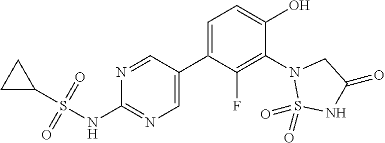 [SMILES: O=C1CN(c2c(O)ccc(-c3cnc(NS(=O)(=O)C4CC4)nc3)c2F)S(=O)(=O)N1] | N-(5-(3-(1,1-dioxido-4-oxo-1,2,5-thiadiazolidin-2- yl)-2-fluoro-4-hydroxyphenyl)pyrimidin-2- yl)cyclopropanesulfonamide | 441.9 |
| 235 | 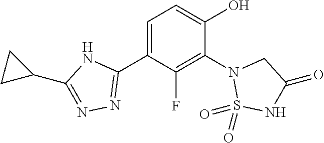 [SMILES: O=C1CN(c2c(O)ccc(-c3nnc(C4CC4)[nH]3)c2F)S(=O)(=O)N1] | 5-(3-(5-cyclopropyl-4H-1,2,4-triazol-3-yl)-2-fluoro- 6-hydroxyphenyl)-1,2,5-thiadiazolidin-3-one 1,1- dioxide | 352.1 |
| 236 | 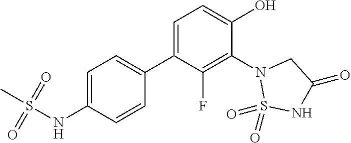 [SMILES: CS(=O)(=O)Nc1ccc(-c2ccc(O)c(N3CC(=O)NS3(=O)=O)c2F)cc1] | N-(3′-(1,1-dioxido-4-oxo-1,2,5-thiadiazolidin-2-yl)- 2′-fluoro-4′-hydroxy-[1,1′-biphenyl]-4- yl)methanesulfonamide | 414.0 |
| 237 | 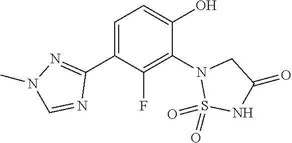 [SMILES: Cn1cnc(-c2ccc(O)c(N3CC(=O)NS3(=O)=O)c2F)n1] | 5-(2-fluoro-6-hydroxy-3-(1-methyl-1H-1,2,4-triazol- 3-yl)phenyl)-1,2,5-thiadiazolidin-3-one 1,1-dioxide | 326.0 |
| 238 | 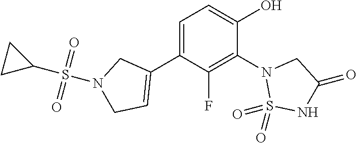 [SMILES: O=C1CN(c2c(O)ccc(C3=CCN(S(=O)(=O)C4CC4)C3)c2F)S(=O)(=O)N1] | 5-(3-(1-(cyclopropylsulfonyl)-2,5-dihydro-1H- pyrrol-3-yl)-2-fluoro-6-hydroxyphenyl)-1,2,5- thiadiazolidin-3-one 1,1-dioxide | 416.1 |
| 239 | 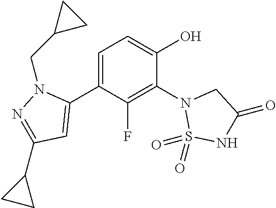 [SMILES: O=C1CN(c2c(O)ccc(-c3cc(C4CC4)nn3CC3CC3)c2F)S(=O)(=O)N1] | 5-(3-(3-cyclopropyl-1-(cyclopropylmethyl)-1H- pyrazol-5-yl)-2-fluoro-6-hydroxyphenyl)-1,2,5- thiadiazolidin-3-one 1,1-dioxide | 404.9 |
| 240 | 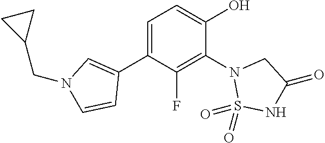 [SMILES: O=C1CN(c2c(O)ccc(-c3ccn(CC4CC4)c3)c2F)S(=O)(=O)N1] | 5-(3-(1-(cyclopropylmethyl)-1H-pyrrol-3-yl)-2- fluoro-6-hydroxyphenyl)-1,2,5-thiadiazolidin-3-one 1,1-dioxide | 363.9 |
| 241 | 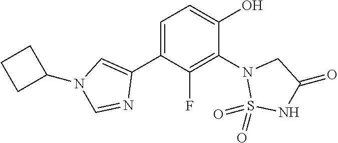 [SMILES: O=C1CN(c2c(O)ccc(-c3cn(C4CCC4)cn3)c2F)S(=O)(=O)N1] | 5-(3-(1-cyclobutyl-1H-imidazol-4-yl)-2-fluoro-6- hydroxyphenyl)-1,2,5-thiadiazolidin-3-one 1,1- dioxide | 365.2 |
| 242 | 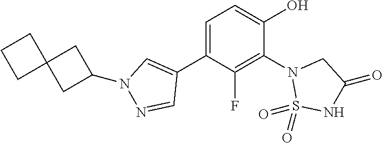 [SMILES: O=C1CN(c2c(O)ccc(-c3cnn(C4CC5(CCC5)C4)c3)c2F)S(=O)(=O)N1] | 5-(2-fluoro-6-hydroxy-3-(1-(spiro[3.3]heptan-2-yl)- 1H-pyrazol-4-yl)phenyl)-1,2,5-thiadiazolidin-3-one 1,1-dioxide | 405.3 |
| 243 | 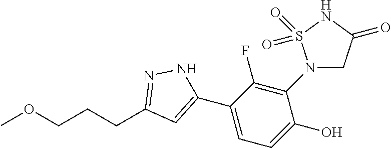 [SMILES: COCCCc1cc(-c2ccc(O)c(N3CC(=O)NS3(=O)=O)c2F)[nH]n1] | 5-(2-fluoro-6-hydroxy-3-(3-(3-methoxypropyl)-1H- pyrazol-5-yl)phenyl)-1,2,5-thiadiazolidin-3-one 1,1- dioxide | 383.2 |
| 244 | 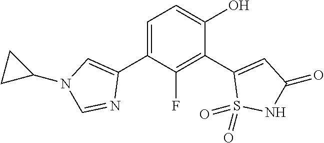 [SMILES: O=C1C=C(c2c(O)ccc(-c3cn(C4CC4)cn3)c2F)S(=O)(=O)N1] | 5-(3-(1-cyclopropyl-1H-imidazol-4-yl)-2-fluoro-6- hydroxyphenyl)isothiazol-3(2H)-one 1,1-dioxide | 348.1 |
| 245 | 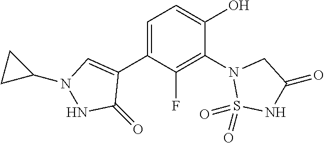 [SMILES: O=C1CN(c2c(O)ccc(-c3cn(C4CC4)[nH]c3=O)c2F)S(=O)(=O)N1] | 5-(3-(1-cyclopropyl-3-oxo-2,3-dihydro-1H-pyrazol- 4-yl)-2-fluoro-6-hydroxyphenyl)-1,2,5- thiadiazolidin-3-one 1,1-dioxide | 367.1 |
| 246 | 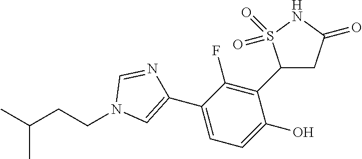 [SMILES: CC(C)CCn1cnc(-c2ccc(O)c(C3CC(=O)NS3(=O)=O)c2F)c1] | 5-(2-fluoro-6-hydroxy-3-(1-isopentyl-1H-imidazol- 4-yl)phenyl)isothiazolidin-3-one 1,1-dioxide | 380.3 |
| 247 | 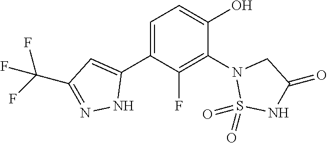 [SMILES: O=C1CN(c2c(O)ccc(-c3cc(C(F)(F)F)n[nH]3)c2F)S(=O)(=O)N1] | 5-(2-fluoro-6-hydroxy-3-(3-(trifluoromethyl)-1H- pyrazol-5-yl)phenyl)-1,2,5-thiadiazolidin-3-one 1,1- dioxide | 378.9 |
| 248 | 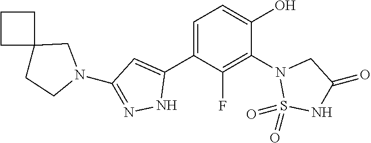 [SMILES: O=C1CN(c2c(O)ccc(-c3cc(N4CCC5(CCC5)C4)n[nH]3)c2F)S(=O)(=O)N1] | 5-(3-(3-(6-azaspiro[3.4]octan-6-yl)-1H-pyrazol-5- yl)-2-fluoro-6-hydroxyphenyl)-1,2,5-thiadiazolidin- 3-one 1,1-dioxide | 420.2 |
| 249 | 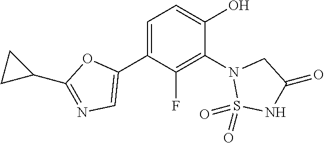 [SMILES: O=C1CN(c2c(O)ccc(-c3cnc(C4CC4)o3)c2F)S(=O)(=O)N1] | 5-(3-(2-cyclopropyloxazol-5-yl)-2-fluoro-6- hydroxyphenyl)-1,2,5-thiadiazolidin-3-one 1,1- dioxide | 352.3 |
| 250 | 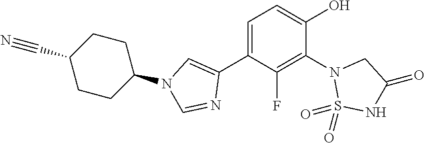 [SMILES: N#C[C@H]1CC[C@H](n2cnc(-c3ccc(O)c(N4CC(=O)NS4(=O)=O)c3F)c2)CC1] | (1r,4r)-4-(4-(3-(1,1-dioxido-4-oxo-1,2,5- thiadiazolidin-2-yl)-2-fluoro-4-hydroxyphenyl)-1H- imidazol-1-yl)cyclohexane-1-carbonitrile | 418.0 |
| 251 | 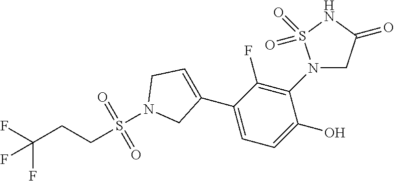 [SMILES: O=C1CN(c2c(O)ccc(C3=CCN(S(=O)(=O)CCC(F)(F)F)C3)c2F)S(=O)(=O)N1] | 5-(2-fluoro-6-hydroxy-3-(1-((3,3,3- trifluoropropyl)sulfonyl)-2,5-dihydro-1H-pyrrol-3- yl)phenyl)-1,2,5-thiadiazolidin-3-one 1,1-dioxide | 472.0 |
| 252 | 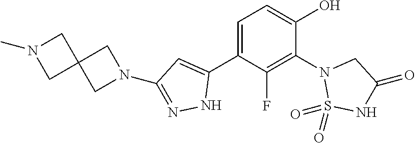 [SMILES: CN1CC2(C1)CN(c1cc(-c3ccc(O)c(N4CC(=O)NS4(=O)=O)c3F)[nH]n1)C2] | 5-(2-fluoro-6-hydroxy-3-(3-(6-methyl-2,6- diazaspiro[3.3]heptan-2-yl)-1H-pyrazol-5- yl)phenyl)-1,2,5-thiadiazolidin-3-one 1,1-dioxide | 420.9 |
| 253 | 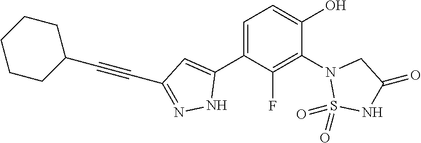 [SMILES: O=C1CN(c2c(O)ccc(-c3cc(C#CC4CCCCC4)n[nH]3)c2F)S(=O)(=O)N1] | 5-(3-(3-(cyclohexylethynyl)-1H-pyrazol-5-yl)-2- fluoro-6-hydroxyphenyl)-1,2,5-thiadiazolidin-3-one 1,1-dioxide | 417.1 |
| 254 | 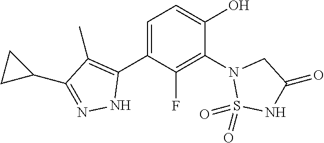 [SMILES: Cc1c(C2CC2)n[nH]c1-c1ccc(O)c(N2CC(=O)NS2(=O)=O)c1F] | 5-(3-(3-cyclopropyl-4-methyl-1H-pyrazol-5-yl)-2- fluoro-6-hydroxyphenyl)-1,2,5-thiadiazolidin-3-one 1,1-dioxide | 365.0 |
| 255 | 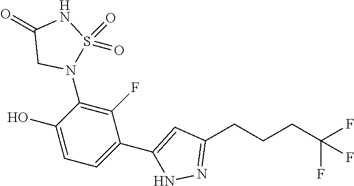 [SMILES: O=C1CN(c2c(O)ccc(-c3cc(CCCC(F)(F)F)n[nH]3)c2F)S(=O)(=O)N1] | 5-(2-fluoro-6-hydroxy-3-(3-(4,4,4-trifluorobutyl)- 1H-pyrazol-5-yl)phenyl)-1,2,5-thiadiazolidin-3-one 1,1-dioxide | 420.9 |
| 256 | 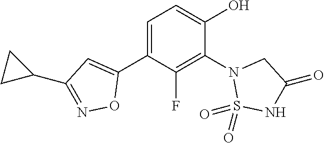 [SMILES: O=C1CN(c2c(O)ccc(-c3cc(C4CC4)no3)c2F)S(=O)(=O)N1] | 5-(3-(3-cyclopropylisoxazol-5-yl)-2-fluoro-6- hydroxyphenyl)-1,2,5-thiadiazolidin-3-one 1,1- dioxide | 352.2 |
| 257 | 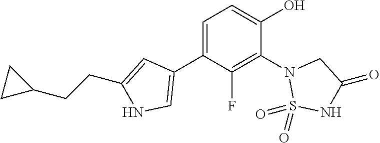 [SMILES: O=C1CN(c2c(O)ccc(-c3c[nH]c(CCC4CC4)c3)c2F)S(=O)(=O)N1] | 5-(3-(5-(2-cyclopropylethyl)-1H-pyrrol-3-yl)-2- fluoro-6-hydroxyphenyl)-1,2,5-thiadiazolidin-3-one 1,1-dioxide | 378.2 |
| 258 | 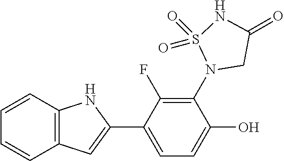 [SMILES: O=C1CN(c2c(O)ccc(-c3cc4ccccc4[nH]3)c2F)S(=O)(=O)N1] | 5-(2-fluoro-6-hydroxy-3-(1H-indol-2-yl)phenyl)- 1,2,5-thiadiazolidin-3-one 1,1-dioxide | 360.1 |
| 259 | 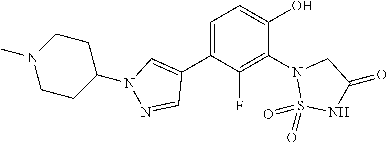 [SMILES: CN1CCC(n2cc(-c3ccc(O)c(N4CC(=O)NS4(=O)=O)c3F)cn2)CC1] | 5-(2-fluoro-6-hydroxy-3-(1-(1-methylpiperidin-4-yl)- 1H-pyrazol-4-yl)phenyl)-1,2,5-thiadiazolidin-3-one 1,1-dioxide | 410.0 |
| 260 | 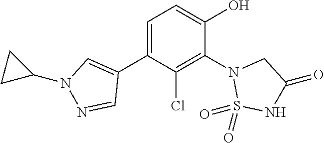 [SMILES: O=C1CN(c2c(O)ccc(-c3cnn(C4CC4)c3)c2Cl)S(=O)(=O)N1] | 5-(2-chloro-3-(1-cyclopropyl-1H-pyrazol-4-yl)-6- hydroxyphenyl)-1,2,5-thiadiazolidin-3-one 1,1- dioxide | 367.0 |
| 261 | 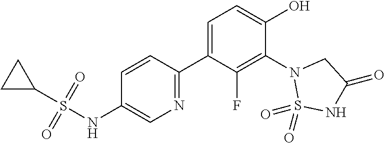 [SMILES: O=C1CN(c2c(O)ccc(-c3ccc(NS(=O)(=O)C4CC4)cn3)c2F)S(=O)(=O)N1] | N-(6-(3-(1,1-dioxido-4-oxo-1,2,5-thiadiazolidin-2- yl)-2-fluoro-4-hydroxyphenyl)pyridin-3- yl)cyclopropanesulfonamide | 441.1 |
| 262 | 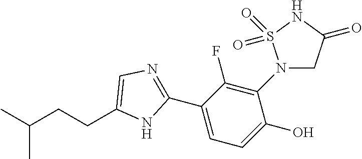 [SMILES: CC(C)CCc1cnc(-c2ccc(O)c(N3CC(=O)NS3(=O)=O)c2F)[nH]1] | 5-(2-fluoro-6-hydroxy-3-(5-isopentyl-1H-imidazol- 2-yl)phenyl)-1,2,5-thiadiazolidin-3-one 1,1-dioxide | 381.0 |
| 263 | 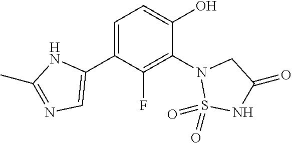 [SMILES: Cc1ncc(-c2ccc(O)c(N3CC(=O)NS3(=O)=O)c2F)[nH]1] | 5-(2-fluoro-6-hydroxy-3-(2-methyl-1H-imidazol-5- yl)phenyl)-1,2,5-thiadiazolidin-3-one 1,1-dioxide | 324.9 |
| 264 | 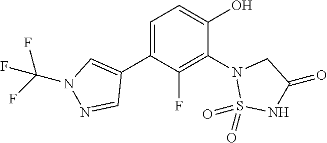 [SMILES: O=C1CN(c2c(O)ccc(-c3cnn(C(F)(F)F)c3)c2F)S(=O)(=O)N1] | 5-(2-fluoro-6-hydroxy-3-(1-(trifluoromethyl)-1H- pyrazol-4-yl)phenyl)-1,2,5-thiadiazolidin-3-one 1,1- dioxide | 379.1 |
| 265 | 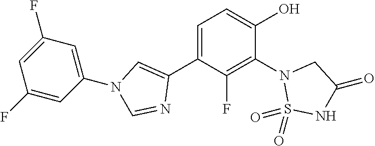 [SMILES: O=C1CN(c2c(O)ccc(-c3cn(-c4cc(F)cc(F)c4)cn3)c2F)S(=O)(=O)N1] | 5-(3-(1-(3,5-difluorophenyl)-1H-imidazol-4-yl)-2- fluoro-6-hydroxyphenyl)-1,2,5-thiadiazolidin-3-one 1,1-dioxide | 423.2 |
| 266 | 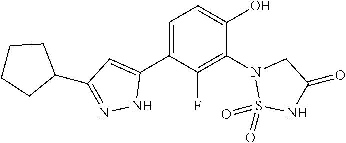 [SMILES: O=C1CN(c2c(O)ccc(-c3cc(C4CCCC4)n[nH]3)c2F)S(=O)(=O)N1] | 5-(3-(3-cyclopentyl-1H-pyrazol-5-yl)-2-fluoro-6- hydroxyphenyl)-1,2,5-thiadiazolidin-3-one 1,1- dioxide | 379.1 |
| 267 | 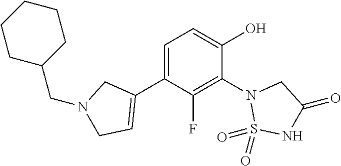 [SMILES: O=C1CN(c2c(O)ccc(C3=CCN(CC4CCCCC4)C3)c2F)S(=O)(=O)N1] | 5-(3-(1-(cyclohexylmethyl)-2,5-dihydro-1H-pyrrol- 3-yl)-2-fluoro-6-hydroxyphenyl)-1,2,5- thiadiazolidin-3-one 1,1-dioxide | 408.0 |
| 268 | 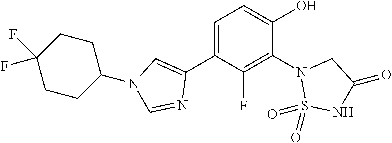 [SMILES: O=C1CN(c2c(O)ccc(-c3cn(C4CCC(F)(F)CC4)cn3)c2F)S(=O)(=O)N1] | 5-(3-(1-(4,4-difluorocyclohexyl)-1H-imidazol-4-yl)- 2-fluoro-6-hydroxyphenyl)-1,2,5-thiadiazolidin-3- one 1,1-dioxide | 431.2 |
| 269 | 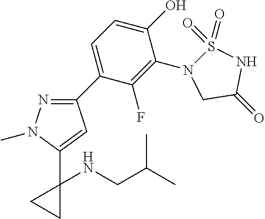 [SMILES: CC(C)CNC1(c2cc(-c3ccc(O)c(N4CC(=O)NS4(=O)=O)c3F)nn2C)CC1] | 5-(2-fluoro-6-hydroxy-3-(5-(1- (isobutylamino)cyclopropyl)-1-methyl-1H-pyrazol- 3-yl)phenyl)-1,2,5-thiadiazolidin-3-one 1,1-dioxide | 435.9 |
| 270 | 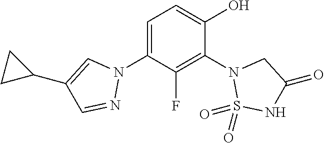 [SMILES: O=C1CN(c2c(O)ccc(-n3cc(C4CC4)cn3)c2F)S(=O)(=O)N1] | 5-(3-(4-cyclopropyl-1H-pyrazol-1-yl)-2-fluoro-6- hydroxyphenyl)-1,2,5-thiadiazolidin-3-one 1,1- dioxide | 353.2 [M + H]⁺ |
| 271 | 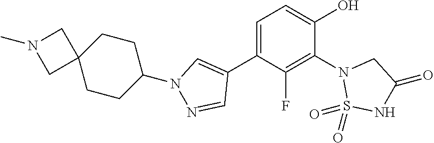 [SMILES: CN1CC2(CCC(n3cc(-c4ccc(O)c(N5CC(=O)NS5(=O)=O)c4F)cn3)CC2)C1] | 5-(2-fluoro-6-hydroxy-3-(1-(2-methyl-2- azaspiro[3.5]nonan-7-yl)-1H-pyrazol-4-yl)phenyl)- 1,2,5-thiadiazolidin-3-one 1,1-dioxide | 448.1 |
| 272 | 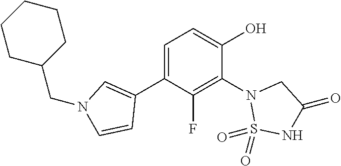 [SMILES: O=C1CN(c2c(O)ccc(-c3ccn(CC4CCCCC4)c3)c2F)S(=O)(=O)N1] | 5-(3-(1-(cyclohexylmethyl)-1H-pyrrol-3-yl)-2- fluoro-6-hydroxyphenyl)-1,2,5-thiadiazolidin-3-one 1,1-dioxide | 406.0 |
| 273 | 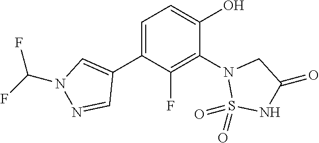 [SMILES: O=C1CN(c2c(O)ccc(-c3cnn(C(F)F)c3)c2F)S(=O)(=O)N1] | 5-(3-(1-(difluoromethyl)-1H-pyrazol-4-yl)-2-fluoro- 6-hydroxyphenyl)-1,2,5-thiadiazolidin-3-one 1,1- dioxide | 361.2 |
| 274 | 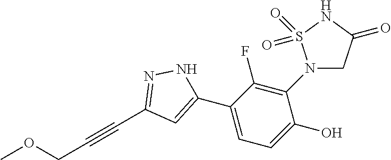 [SMILES: COCC#Cc1cc(-c2ccc(O)c(N3CC(=O)NS3(=O)=O)c2F)[nH]n1] | 5-(2-fluoro-6-hydroxy-3-(3-(3-methoxyprop-1-yn-1- yl)-1H-pyrazol-5-yl)phenyl)-1,2,5-thiadiazolidin-3- one 1,1-dioxide | 379.0 |
| 275 | 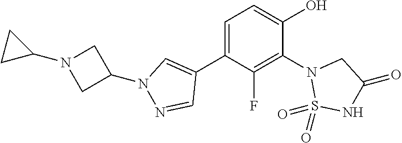 [SMILES: O=C1CN(c2c(O)ccc(-c3cnn(C4CN(C5CC5)C4)c3)c2F)S(=O)(=O)N1] | 5-(3-(1-(1-cyclopropylazetidin-3-yl)-1H-pyrazol-4- yl)-2-fluoro-6-hydroxyphenyl)-1,2,5-thiadiazolidin- 3-one 1,1-dioxide | 406.3 |
| 276 | 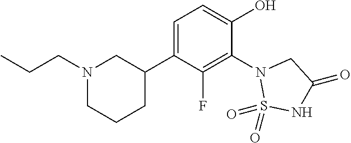 [SMILES: CCCN1CCCC(c2ccc(O)c(N3CC(=O)NS3(=O)=O)c2F)C1] | 5-(2-fluoro-6-hydroxy-3-(1-propylpiperidin-3- yl)phenyl)-1,2,5-thiadiazolidin-3-one 1,1-dioxide | 370.2 |
| 277 | 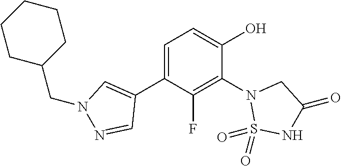 [SMILES: O=C1CN(c2c(O)ccc(-c3cnn(CC4CCCCC4)c3)c2F)S(=O)(=O)N1] | 5-(3-(1-(cyclohexylmethyl)-1H-imidazol-4-yl)-2- fluoro-6-hydroxyphenyl)isothiazol-3(2H)-one 1,1- dioxide | 404.1 |
| 278 | 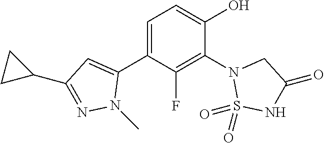 [SMILES: Cn1nc(C2CC2)cc1-c1ccc(O)c(N2CC(=O)NS2(=O)=O)c1F] | 5-(3-(3-cyclopropyl-1-methyl-1H-pyrazol-5-yl)-2- fluoro-6-hydroxyphenyl)-1,2,5-thiadiazolidin-3-one 1,1-dioxide | 365.2 |
| 279 | 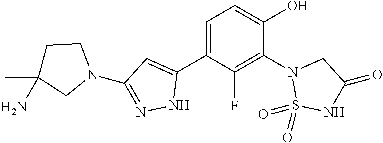 [SMILES: CC1(N)CCN(c2cc(-c3ccc(O)c(N4CC(=O)NS4(=O)=O)c3F)[nH]n2)C1] | 5-(3-(3-(3-amino-3-methylpyrrolidin-1-yl)-1H- pyrazol-5-yl)-2-fluoro-6-hydroxyphenyl)-1,2,5- thiadiazolidin-3-one 1,1-dioxide | 409.1 |
| 280 | 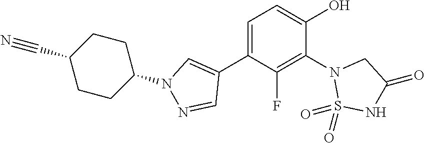 [SMILES: N#C[C@H]1CC[C@@H](n2cc(-c3ccc(O)c(N4CC(=O)NS4(=O)=O)c3F)cn2)CC1] | (1s,4s)-4-(4-(3-(1,1-dioxido-4-oxo-1,2,5- thiadiazolidin-2-yl)-2-fluoro-4-hydroxyphenyl)-1H- pyrazol-1-yl)cyclohexane-1-carbonitrile | 418.1 |
| 281 | 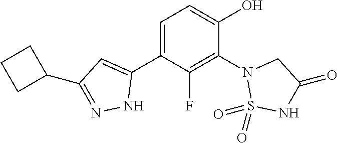 [SMILES: O=C1CN(c2c(O)ccc(-c3cc(C4CCC4)n[nH]3)c2F)S(=O)(=O)N1] | 5-(3-(3-cyclobutyl-1H-pyrazol-5-yl)-2-fluoro-6- hydroxyphenyl)-1,2,5-thiadiazolidin-3-one 1,1- dioxide | 367.2 [M + H]⁺ |
| 282 | 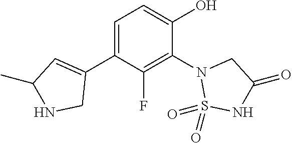 [SMILES: CC1C=C(c2ccc(O)c(N3CC(=O)NS3(=O)=O)c2F)CN1] | 5-(2-fluoro-6-hydroxy-3-(5-methyl-2,5-dihydro-1H- pyrrol-3-yl)phenyl)-1,2,5-thiadiazolidin-3-one 1,1- dioxide | 326.0 |
| 283 | 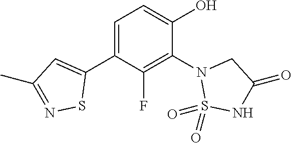 [SMILES: Cc1cc(-c2ccc(O)c(N3CC(=O)NS3(=O)=O)c2F)sn1] | 5-(2-fluoro-6-hydroxy-3-(3-methylisothiazol-5- yl)phenyl)-1,2,5-thiadiazolidin-3-one 1,1-dioxide | 342.2 |
| 284 | 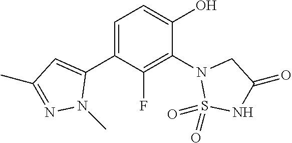 [SMILES: Cc1cc(-c2ccc(O)c(N3CC(=O)NS3(=O)=O)c2F)n(C)n1] | 5-(3-(1,3-dimethyl-1H-pyrazol-5-yl)-2-fluoro-6- hydroxyphenyl)-1,2,5-thiadiazolidin-3-one 1,1- dioxide | 339.0 |
| 285 | 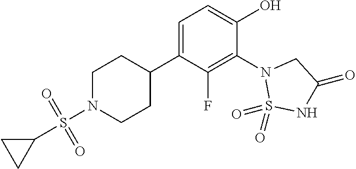 [SMILES: O=C1CN(c2c(O)ccc(C3CCN(S(=O)(=O)C4CC4)CC3)c2F)S(=O)(=O)N1] | 5-(3-(1-(cyclopropylsulfonyl)piperidin-4-yl)-2- fluoro-6-hydroxyphenyl)-1,2,5-thiadiazolidin-3-one 1,1-dioxide | 432.1 |
| 286 | 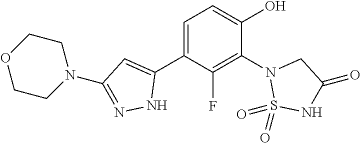 [SMILES: O=C1CN(c2c(O)ccc(-c3cc(N4CCOCC4)n[nH]3)c2F)S(=O)(=O)N1] | 5-(2-fluoro-6-hydroxy-3-(3-morpholino-1H-pyrazol- 5-yl)phenyl)-1,2,5-thiadiazolidin-3-one 1,1-dioxide | 396.2 |
| 287 | 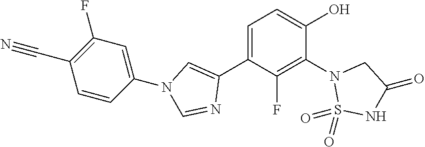 [SMILES: N#Cc1ccc(-n2cnc(-c3ccc(O)c(N4CC(=O)NS4(=O)=O)c3F)c2)cc1F] | 4-(4-(3-(1,1-dioxido-4-oxo-1,2,5-thiadiazolidin-2- yl)-2-fluoro-4-hydroxyphenyl)-1H-imidazol-1-yl)-2- fluorobenzonitrile | 430.1 |
| 288 | 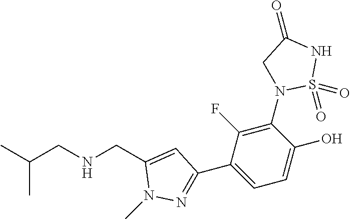 [SMILES: CC(C)CNCc1cc(-c2ccc(O)c(N3CC(=O)NS3(=O)=O)c2F)nn1C] | 5-(2-fluoro-6-hydroxy-3-(5-((isobutylamino)methyl)- 1-methyl-1H-pyrazol-3-yl)phenyl)-1,2,5- thiadiazolidin-3-one 1,1-dioxide | 410.1 |
| 289 | 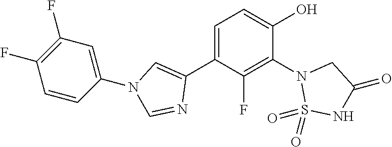 [SMILES: O=C1CN(c2c(O)ccc(-c3cn(-c4ccc(F)c(F)c4)cn3)c2F)S(=O)(=O)N1] | 5-(3-(1-(3,4-difluorophenyl)-1H-imidazol-4-yl)-2- fluoro-6-hydroxyphenyl)-1,2,5-thiadiazolidin-3-one 1,1-dioxide | 423.1 |
| 290 | 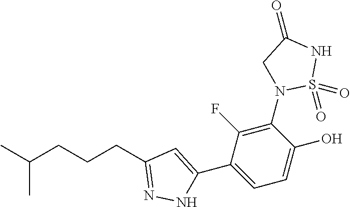 [SMILES: CC(C)CCCc1cc(-c2ccc(O)c(N3CC(=O)NS3(=O)=O)c2F)[nH]n1] | 5-(2-fluoro-6-hydroxy-3-(3-(4-methylpentyl)-1H- pyrazol-5-yl)phenyl)-1,2,5-thiadiazolidin-3-one 1,1- dioxide | 395.3 |
| 291 | 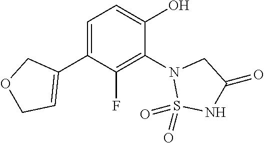 [SMILES: O=C1CN(c2c(O)ccc(C3=CCOC3)c2F)S(=O)(=O)N1] | 5-(3-(2,5-dihydrofuran-3-yl)-2-fluoro-6- hydroxyphenyl)-1,2,5-thiadiazolidin-3-one 1,1- dioxide | 313.0 |
| 292 | 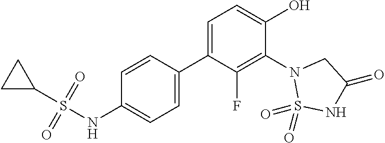 [SMILES: O=C1CN(c2c(O)ccc(-c3ccc(NS(=O)(=O)C4CC4)cc3)c2F)S(=O)(=O)N1] | N-(3′-(1,1-dioxido-4-oxo-1,2,5-thiadiazolidin-2-yl)- 2′-fluoro-4′-hydroxy-[1,1′-biphenyl]-4- yl)cyclopropanesulfonamide | 440.1 |
| 293 | 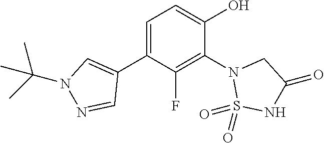 [SMILES: CC(C)(C)n1cc(-c2ccc(O)c(N3CC(=O)NS3(=O)=O)c2F)cn1] | 5-(3-(1-(tert-butyl)-1H-pyrazol-4-yl)-2-fluoro-6- hydroxyphenyl)-1,2,5-thiadiazolidin-3-one 1,1- dioxide | 367.0 |
| 294 | 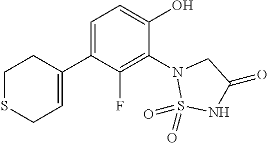 [SMILES: O=C1CN(c2c(O)ccc(C3=CCSCC3)c2F)S(=O)(=O)N1] | 5-(3-(3,6-dihydro-2H-thiopyran-4-yl)-2-fluoro-6- hydroxyphenyl)-1,2,5-thiadiazolidin-3-one 1,1- dioxide | 343.3 |
| 295 | 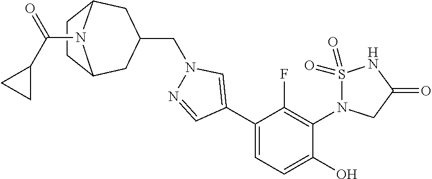 [SMILES: O=C1CN(c2c(O)ccc(-c3cnn(CC4CC5CCC(C4)N5C(=O)C4CC4)c3)c2F)S(=O)(=O)N1] | 5-(3-(1-((8-(cyclopropanecarbonyl)-8- azabicyclo[3.2.1]octan-3-yl)methyl)-1H-pyrazol-4- yl)-2-fluoro-6-hydroxyphenyl)-1,2,5-thiadiazolidin- 3-one 1,1-dioxide | 502.3 |
| 296 | 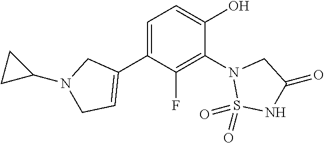 [SMILES: O=C1CN(c2c(O)ccc(C3=CCN(C4CC4)C3)c2F)S(=O)(=O)N1] | 5-(3-(1-cyclopropyl-2,5-dihydro-1H-pyrrol-3-yl)-2- fluoro-6-hydroxyphenyl)-1,2,5-thiadiazolidin-3-one 1,1-dioxide | 351.9 |
| 297 | 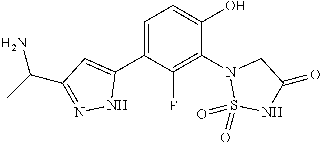 [SMILES: CC(N)c1cc(-c2ccc(O)c(N3CC(=O)NS3(=O)=O)c2F)[nH]n1] | 5-(3-(3-(1-aminoethyl)-1H-pyrazol-5-yl)-2-fluoro-6- hydroxyphenyl)-1,2,5-thiadiazolidin-3-one 1,1- dioxide | 353.9 |
| 298 | 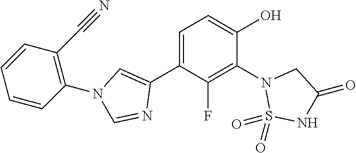 [SMILES: N#Cc1ccccc1-n1cnc(-c2ccc(O)c(N3CC(=O)NS3(=O)=O)c2F)c1] | 2-(4-(3-(1,1-dioxido-4-oxo-1,2,5-thiadiazolidin-2- yl)-2-fluoro-4-hydroxyphenyl)-1H-imidazol-1- yl)benzonitrile | 412.1 |
| 299 | 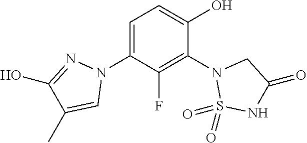 [SMILES: Cc1cn(-c2ccc(O)c(N3CC(=O)NS3(=O)=O)c2F)nc1O] | 5-(2-fluoro-6-hydroxy-3-(3-hydroxy-4-methyl-1H- pyrazol-1-yl)phenyl)-1,2,5-thiadiazolidin-3-one 1,1- dioxide | 341.0 |
| 300 | 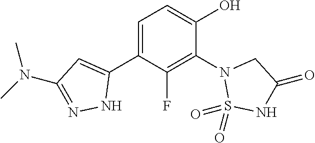 [SMILES: CN(C)c1cc(-c2ccc(O)c(N3CC(=O)NS3(=O)=O)c2F)[nH]n1] | 5-(3-(3-(dimethylamino)-1H-pyrazol-5-yl)-2-fluoro- 6-hydroxyphenyl)-1,2,5-thiadiazolidin-3-one 1,1- dioxide | 354.3 |
| 301 | 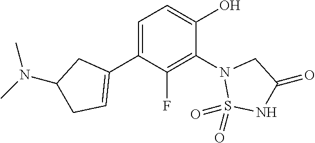 [SMILES: CN(C)C1CC=C(c2ccc(O)c(N3CC(=O)NS3(=O)=O)c2F)C1] | 5-(3-(4-(dimethylamino)cyclopent-1-en-1-yl)-2- fluoro-6-hydroxyphenyl)-1,2,5-thiadiazolidin-3-one 1,1-dioxide | 354.3 |
| 302 | 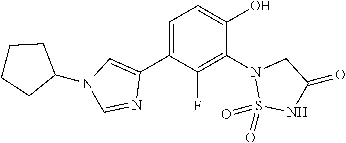 [SMILES: O=C1CN(c2c(O)ccc(-c3cn(C4CCCC4)cn3)c2F)S(=O)(=O)N1] | 5-(3-(1-cyclopentyl-1H-imidazol-4-yl)-2-fluoro-6- hydroxyphenyl)-1,2,5-thiadiazolidin-3-one 1,1- dioxide | 379.2 |
| 303 | 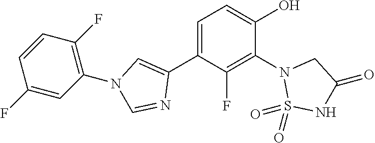 [SMILES: O=C1CN(c2c(O)ccc(-c3cn(-c4cc(F)ccc4F)cn3)c2F)S(=O)(=O)N1] | 5-(3-(1-(2,5-difluorophenyl)-1H-imidazol-4-yl)-2- fluoro-6-hydroxyphenyl)-1,2,5-thiadiazolidin-3-one 1,1-dioxide | 423.1 |
| 304 | 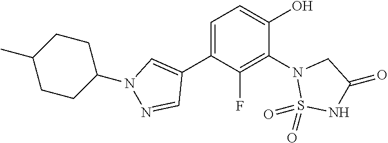 [SMILES: CC1CCC(n2cc(-c3ccc(O)c(N4CC(=O)NS4(=O)=O)c3F)cn2)CC1] | 5-(2-fluoro-6-hydroxy-3-(1-(4-methylcyclohexyl)- 1H-pyrazol-4-yl)phenyl)-1,2,5-thiadiazolidin-3-one 1,1-dioxide | 407.1 |
| 305 | 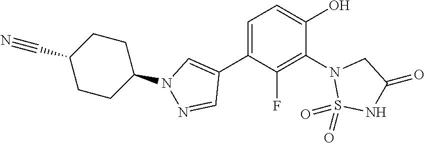 [SMILES: N#C[C@H]1CC[C@H](n2cc(-c3ccc(O)c(N4CC(=O)NS4(=O)=O)c3F)cn2)CC1] | (1r,4r)-4-(4-(3-(1,1-dioxido-4-oxo-1,2,5- thiadiazolidin-2-yl)-2-fluoro-4-hydroxyphenyl)-1H- pyrazol-1-yl)cyclohexane-1-carbonitrile | 418.0 |
| 306 | 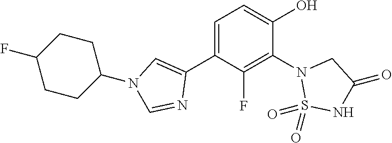 [SMILES: O=C1CN(c2c(O)ccc(-c3cn(C4CCC(F)CC4)cn3)c2F)S(=O)(=O)N1] | 5-(2-fluoro-3-(1-(4-fluorocyclohexyl)-1H-imidazol- 4-yl)-6-hydroxyphenyl)-1,2,5-thiadiazolidin-3-one 1,1-dioxide | 411.1 |
| 307 | 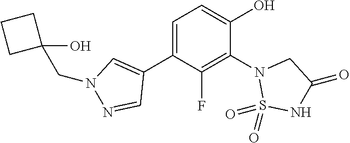 [SMILES: O=C1CN(c2c(O)ccc(-c3cnn(CC4(O)CCC4)c3)c2F)S(=O)(=O)N1] | 5-(2-fluoro-6-hydroxy-3-(1-((1- hydroxycyclobutyl)methyl)-1H-pyrazol-4- yl)phenyl)-1,2,5-thiadiazolidin-3-one 1,1-dioxide | 394.9 |
| 308 | 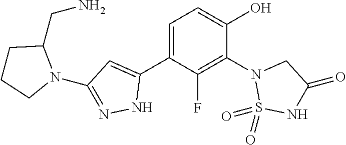 [SMILES: NCC1CCCN1c1cc(-c2ccc(O)c(N3CC(=O)NS3(=O)=O)c2F)[nH]n1] | 5-(3-(3-(2-(aminomethyl)pyrrolidin-1-yl)-1H- pyrazol-5-yl)-2-fluoro-6-hydroxyphenyl)-1,2,5- thiadiazolidin-3-one 1,1-dioxide | 409.0 |
| 309 | 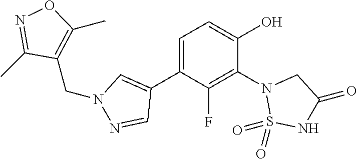 [SMILES: Cc1noc(C)c1Cn1cc(-c2ccc(O)c(N3CC(=O)NS3(=O)=O)c2F)cn1] | 5-(3-(1-((3,5-dimethylisoxazol-4-yl)methyl)-1H- pyrazol-4-yl)-2-fluoro-6-hydroxyphenyl)-1,2,5- thiadiazolidin-3-one 1,1-dioxide | 420.0 |
| 310 | 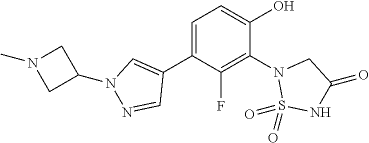 [SMILES: CN1CC(n2cc(-c3ccc(O)c(N4CC(=O)NS4(=O)=O)c3F)cn2)C1] | 5-(2-fluoro-6-hydroxy-3-(1-(1-methylazetidin-3-yl)- 1H-pyrazol-4-yl)phenyl)-1,2,5-thiadiazolidin-3-one 1,1-dioxide | 380.3 |
| 311 | 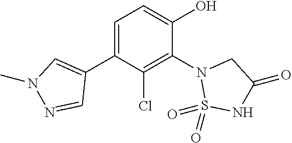 [SMILES: Cn1cc(-c2ccc(O)c(N3CC(=O)NS3(=O)=O)c2Cl)cn1] | 5-(2-chloro-6-hydroxy-3-(1-methyl-1H-pyrazol-4- yl)phenyl)-1,2,5-thiadiazolidin-3-one 1,1-dioxide | 341.0 |
| 312 | 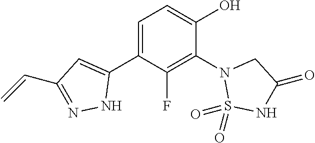 [SMILES: C=Cc1cc(-c2ccc(O)c(N3CC(=O)NS3(=O)=O)c2F)[nH]n1] | 5-(2-fluoro-6-hydroxy-3-(3-vinyl-1H-pyrazol-5- yl)phenyl)-1,2,5-thiadiazolidin-3-one 1,1-dioxide | 337.1 |
| 313 | 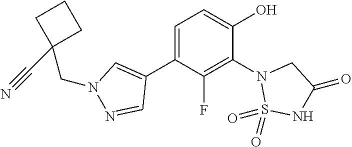 [SMILES: N#CC1(Cn2cc(-c3ccc(O)c(N4CC(=O)NS4(=O)=O)c3F)cn2)CCC1] | 1-((4-(3-(1,1-dioxido-4-oxo-1,2,5-thiadiazolidin-2- yl)-2-fluoro-4-hydroxyphenyl)-1H-pyrazol-1- yl)methyl)cyclobutane-1-carbonitrile | 404.1 |
| 314 | 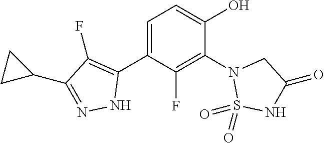 [SMILES: O=C1CN(c2c(O)ccc(-c3[nH]nc(C4CC4)c3F)c2F)S(=O)(=O)N1] | 5-(3-(3-cyclopropyl-4-fluoro-1H-pyrazol-5-yl)-2- fluoro-6-hydroxyphenyl)-1,2,5-thiadiazolidin-3-one 1,1-dioxide | 369.2 |
| 315 | 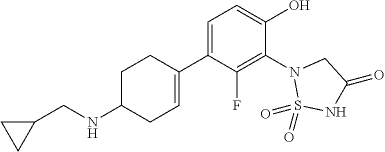 [SMILES: O=C1CN(c2c(O)ccc(C3=CCC(NCC4CC4)CC3)c2F)S(=O)(=O)N1] | 5-(4′-((cyclopropylmethyl)amino)-2-fluoro-4- hydroxy-2′,3′,4′,5′-tetrahydro-[1,1′-biphenyl]-3-yl)- 1,2,5-thiadiazolidin-3-one 1,1-dioxide | 394.3 |
| 316 | 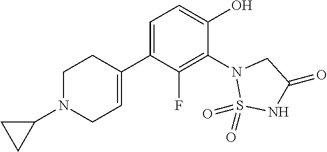 [SMILES: O=C1CN(c2c(O)ccc(C3=CCN(C4CC4)CC3)c2F)S(=O)(=O)N1] | 5-(3-(1-cyclopropyl-1,2,3,6-tetrahydropyridin-4-yl)- 2-fluoro-6-hydroxyphenyl)-1,2,5-thiadiazolidin-3- one 1,1-dioxide | 366.2 |
| 317 | 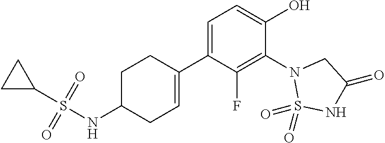 [SMILES: O=C1CN(c2c(O)ccc(C3=CCC(NS(=O)(=O)C4CC4)CC3)c2F)S(=O)(=O)N1] | N-(3′-(1,1-dioxido-4-oxo-1,2,5-thiadiazolidin-2-yl)- 2′-fluoro-4′-hydroxy-2,3,4,5-tetrahydro-[1,1′- biphenyl]-4-yl)cyclopropanesulfonamide | 444.2 |
| 318 | 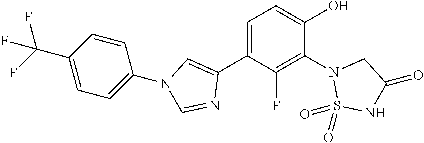 [SMILES: O=C1CN(c2c(O)ccc(-c3cn(-c4ccc(C(F)(F)F)cc4)cn3)c2F)S(=O)(=O)N1] | 5-(2-fluoro-6-hydroxy-3-(1-(4- (trifluoromethyl)phenyl)-1H-imidazol-4-yl)phenyl)- 1,2,5-thiadiazolidin-3-one 1,1-dioxide | 455.0 |
| 319 | 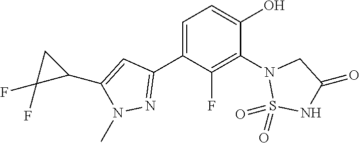 [SMILES: Cn1nc(-c2ccc(O)c(N3CC(=O)NS3(=O)=O)c2F)cc1C1CC1(F)F] | 5-(3-(5-(2,2-difluorocyclopropyl)-1-methyl-1H- pyrazol-3-yl)-2-fluoro-6-hydroxyphenyl)-1,2,5- thiadiazolidin-3-one 1,1-dioxide | 401.1 |
| 320 | 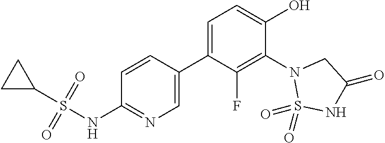 [SMILES: O=C1CN(c2c(O)ccc(-c3ccc(NS(=O)(=O)C4CC4)nc3)c2F)S(=O)(=O)N1] | N-(5-(3-(1,1-dioxido-4-oxo-1,2,5-thiadiazolidin-2- yl)-2-fluoro-4-hydroxyphenyl)pyridin-2- yl)cyclopropanesulfonamide | 441.0 |
| 321 | 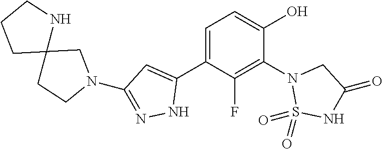 [SMILES: O=C1CN(c2c(O)ccc(-c3cc(N4CCC5(CCCN5)C4)n[nH]3)c2F)S(=O)(=O)N1] | 5-(3-(3-(1,7-diazaspiro[4.4]nonan-7-yl)-1H-pyrazol- 5-yl)-2-fluoro-6-hydroxyphenyl)-1,2,5- thiadiazolidin-3-one 1,1-dioxide | 435.1 |
| 322 | 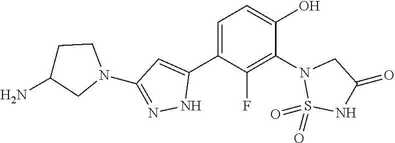 [SMILES: NC1CCN(c2cc(-c3ccc(O)c(N4CC(=O)NS4(=O)=O)c3F)[nH]n2)C1] | 5-(3-(3-(3-aminopyrrolidin-1-yl)-1H-pyrazol-5-yl)-2- fluoro-6-hydroxyphenyl)-1,2,5-thiadiazolidin-3-one 1,1-dioxide | 395.0 |
| 323 | 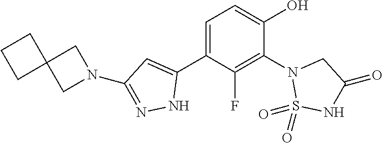 [SMILES: O=C1CN(c2c(O)ccc(-c3cc(N4CC5(CCC5)C4)n[nH]3)c2F)S(=O)(=O)N1] | 5-(3-(3-(2-azaspiro[3.3]heptan-2-yl)-1H-pyrazol-5- yl)-2-fluoro-6-hydroxyphenyl)-1,2,5-thiadiazolidin- 3-one 1,1-dioxide | 406.0 |
| 324 | 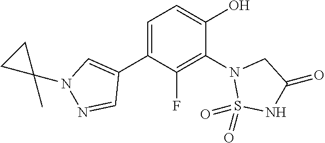 [SMILES: CC1(n2cc(-c3ccc(O)c(N4CC(=O)NS4(=O)=O)c3F)cn2)CC1] | 5-(2-fluoro-6-hydroxy-3-(1-(1-methylcyclopropyl)- 1H-pyrazol-4-yl)phenyl)-1,2,5-thiadiazolidin-3-one 1,1-dioxide | 365.0 |
| 325 | 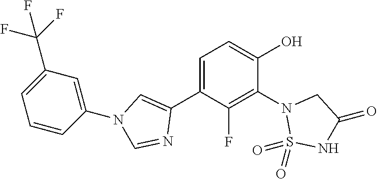 [SMILES: O=C1CN(c2c(O)ccc(-c3cn(-c4cccc(C(F)(F)F)c4)cn3)c2F)S(=O)(=O)N1] | 5-(2-fluoro-6-hydroxy-3-(1-(3- (trifluoromethyl)phenyl)-1H-imidazol-4-yl)phenyl)- 1,2,5-thiadiazolidin-3-one 1,1-dioxide | 455.0 |
| 326 | 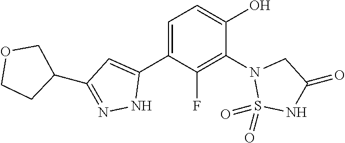 [SMILES: O=C1CN(c2c(O)ccc(-c3cc(C4CCOC4)n[nH]3)c2F)S(=O)(=O)N1] | 5-(2-fluoro-6-hydroxy-3-(3-(tetrahydrofuran-3-yl)- 1H-pyrazol-5-yl)phenyl)-1,2,5-thiadiazolidin-3-one 1,1-dioxide | 381.2 |
| 327 | 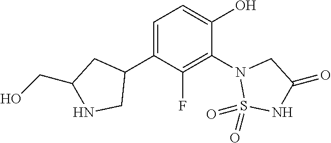 [SMILES: O=C1CN(c2c(O)ccc(C3CNC(CO)C3)c2F)S(=O)(=O)N1] | 5-(2-fluoro-6-hydroxy-3-(5- (hydroxymethyl)pyrrolidin-3-yl)phenyl)-1,2,5- thiadiazolidin-3-one 1,1-dioxide | 344.0 |
| 328 | 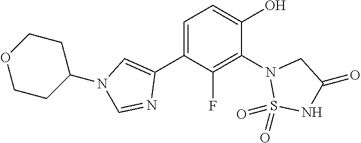 [SMILES: O=C1CN(c2c(O)ccc(-c3cn(C4CCOCC4)cn3)c2F)S(=O)(=O)N1] | 5-(2-fluoro-6-hydroxy-3-(1-(tetrahydro-2H-pyran-4- yl)-1H-imidazol-4-yl)phenyl)-1,2,5-thiadiazolidin-3- one 1,1-dioxide | 395.0 |
| 329 | 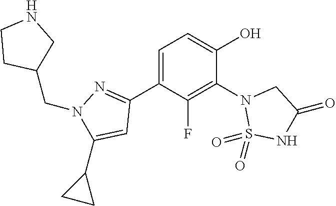 [SMILES: O=C1CN(c2c(O)ccc(-c3cc(C4CC4)n(CC4CCNC4)n3)c2F)S(=O)(=O)N1] | 5-(3-(5-cyclopropyl-1-(pyrrolidin-3-ylmethyl)-1H- pyrazol-3-yl)-2-fluoro-6-hydroxyphenyl)-1,2,5- thiadiazolidin-3-one 1,1-dioxide | 434.2 |
| 330 | 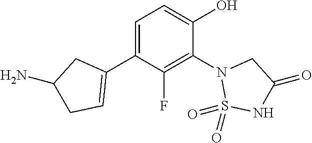 [SMILES: NC1CC=C(c2ccc(O)c(N3CC(=O)NS3(=O)=O)c2F)C1] | 5-(3-(4-aminocyclopent-1-en-1-yl)-2-fluoro-6- hydroxyphenyl)-1,2,5-thiadiazolidin-3-one 1,1- dioxide | 326.3 |
| 331 | 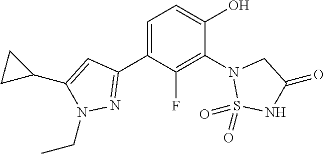 [SMILES: CCn1nc(-c2ccc(O)c(N3CC(=O)NS3(=O)=O)c2F)cc1C1CC1] | 5-(3-(5-cyclopropyl-1-ethyl-1H-pyrazol-3-yl)-2- fluoro-6-hydroxyphenyl)-1,2,5-thiadiazolidin-3-one 1,1-dioxide | 379.2 |
| 332 | 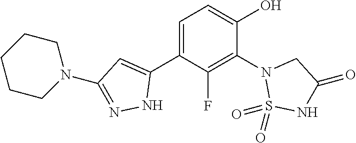 [SMILES: O=C1CN(c2c(O)ccc(-c3cc(N4CCCCC4)n[nH]3)c2F)S(=O)(=O)N1] | 5-(2-fluoro-6-hydroxy-3-(3-(piperidin-1-yl)-1H- pyrazol-5-yl)phenyl)-1,2,5-thiadiazolidin-3-one 1,1- dioxide | 394.3 |
| 333 | 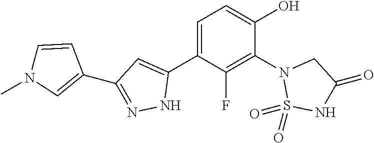 [SMILES: Cn1ccc(-c2cc(-c3ccc(O)c(N4CC(=O)NS4(=O)=O)c3F)[nH]n2)c1] | 5-(2-fluoro-6-hydroxy-3-(3-(1-methyl-1H-pyrrol-3- yl)-1H-pyrazol-5-yl)phenyl)-1,2,5-thiadiazolidin-3- one 1,1-dioxide | 390.2 |
| 334 | 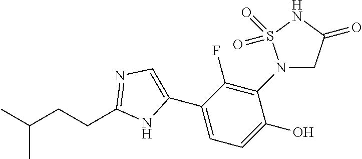 [SMILES: CC(C)CCc1ncc(-c2ccc(O)c(N3CC(=O)NS3(=O)=O)c2F)[nH]1] | 5-(2-fluoro-6-hydroxy-3-(2-isopentyl-1H-imidazol- 5-yl)phenyl)-1,2,5-thiadiazolidin-3-one 1,1-dioxide | 380.9 |
| 335 | 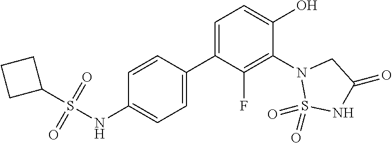 [SMILES: O=C1CN(c2c(O)ccc(-c3ccc(NS(=O)(=O)C4CCC4)cc3)c2F)S(=O)(=O)N1] | N-(3′-(1,1-dioxido-4-oxo-1,2,5-thiadiazolidin-2-yl)- 2′-fluoro-4′-hydroxy-[1,1′-biphenyl]-4- yl)cyclobutanesulfonamide | 454.1 |
| 336 | 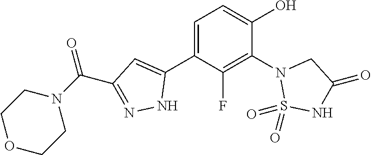 [SMILES: O=C1CN(c2c(O)ccc(-c3cc(C(=O)N4CCOCC4)n[nH]3)c2F)S(=O)(=O)N1] | 5-(2-fluoro-6-hydroxy-3-(3-(morpholine-4- carbonyl)-1H-pyrazol-5-yl)phenyl)-1,2,5- thiadiazolidin-3-one 1,1-dioxide | 424.1 |
| 337 | 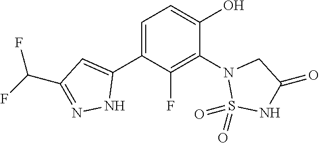 [SMILES: O=C1CN(c2c(O)ccc(-c3cc(C(F)F)n[nH]3)c2F)S(=O)(=O)N1] | 5-(3-(3-(difluoromethyl)-1H-pyrazol-5-yl)-2-fluoro- 6-hydroxyphenyl)-1,2,5-thiadiazolidin-3-one 1,1- dioxide | 361.1 |
| 338 | 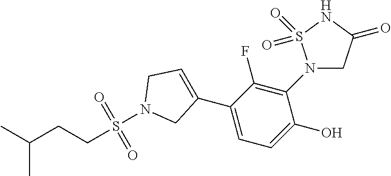 [SMILES: CC(C)CCS(=O)(=O)N1CC=C(c2ccc(O)c(N3CC(=O)NS3(=O)=O)c2F)C1] | 5-(2-fluoro-6-hydroxy-3-(1-(isopentylsulfonyl)-2,5- dihydro-1H-pyrrol-3-yl)phenyl)-1,2,5-thiadiazolidin- 3-one 1,1-dioxide | 445.9 |
| 339 | 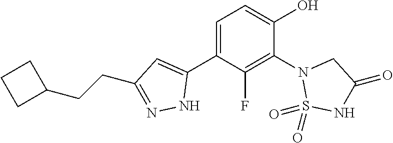 [SMILES: O=C1CN(c2c(O)ccc(-c3cc(CCC4CCC4)n[nH]3)c2F)S(=O)(=O)N1] | 5-(3-(3-(2-cyclobutylethyl)-1H-pyrazol-5-yl)-2- fluoro-6-hydroxyphenyl)-1,2,5-thiadiazolidin-3-one 1,1-dioxide | 393.1 |
| 340 | 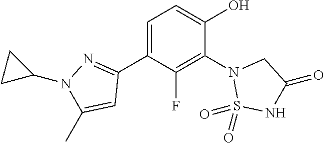 [SMILES: Cc1cc(-c2ccc(O)c(N3CC(=O)NS3(=O)=O)c2F)nn1C1CC1] | 5-(3-(1-cyclopropyl-5-methyl-1H-pyrazol-3-yl)-2- fluoro-6-hydroxyphenyl)-1,2,5-thiadiazolidin-3-one 1,1-dioxide | 365.1 |
| 341 | 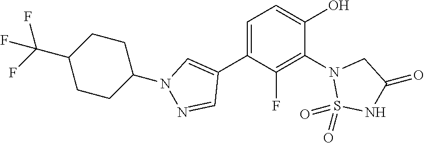 [SMILES: O=C1CN(c2c(O)ccc(-c3cnn(C4CCC(C(F)(F)F)CC4)c3)c2F)S(=O)(=O)N1] | 5-(2-fluoro-6-hydroxy-3-(1-(4- (trifluoromethyl)cyclohexyl)-1H-pyrazol-4- yl)phenyl)-1,2,5-thiadiazolidin-3-one 1,1-dioxide | 461.2 |
| 342 | 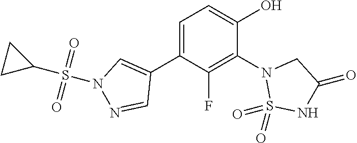 [SMILES: O=C1CN(c2c(O)ccc(-c3cnn(S(=O)(=O)C4CC4)c3)c2F)S(=O)(=O)N1] | 5-(3-(1-(cyclopropylsulfonyl)-1H-pyrazol-4-yl)-2- fluoro-6-hydroxyphenyl)-1,2,5-thiadiazolidin-3-one 1,1-dioxide | 415.0 |
| 343 | 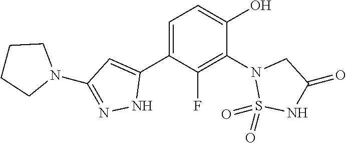 [SMILES: O=C1CN(c2c(O)ccc(-c3cc(N4CCCC4)n[nH]3)c2F)S(=O)(=O)N1] | 5-(2-fluoro-6-hydroxy-3-(3-(pyrrolidin-1-yl)-1H- pyrazol-5-yl)phenyl)-1,2,5-thiadiazolidin-3-one 1,1- dioxide | 380.2 |
| 344 | 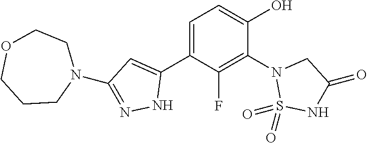 [SMILES: O=C1CN(c2c(O)ccc(-c3cc(N4CCCOCC4)n[nH]3)c2F)S(=O)(=O)N1] | 5-(3-(3-(1,4-oxazepan-4-yl)-1H-pyrazol-5-yl)-2- fluoro-6-hydroxyphenyl)-1,2,5-thiadiazolidin-3-one 1,1-dioxide | 410.1 |
| 345 | 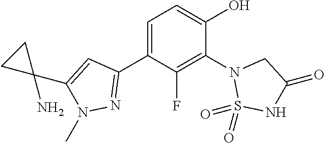 [SMILES: Cn1nc(-c2ccc(O)c(N3CC(=O)NS3(=O)=O)c2F)cc1C1(N)CC1] | 5-(3-(5-(1-aminocyclopropyl)-1-methyl-1H-pyrazol- 3-yl)-2-fluoro-6-hydroxyphenyl)-1,2,5- thiadiazolidin-3-one 1,1-dioxide | 380.2 |
| 346 | 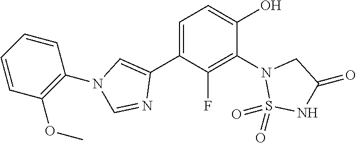 [SMILES: COc1ccccc1-n1cnc(-c2ccc(O)c(N3CC(=O)NS3(=O)=O)c2F)c1] | 5-(2-fluoro-6-hydroxy-3-(1-(2-methoxyphenyl)-1H- imidazol-4-yl)phenyl)-1,2,5-thiadiazolidin-3-one 1,1- dioxide | 417.0 |
| 347 | 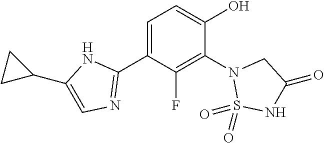 [SMILES: O=C1CN(c2c(O)ccc(-c3ncc(C4CC4)[nH]3)c2F)S(=O)(=O)N1] | 5-(3-(5-cyclopropyl-1H-imidazol-2-yl)-2-fluoro-6- hydroxyphenyl)-1,2,5-thiadiazolidin-3-one 1,1- dioxide | 351.0 |
| 348 | 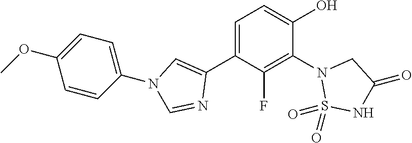 [SMILES: COc1ccc(-n2cnc(-c3ccc(O)c(N4CC(=O)NS4(=O)=O)c3F)c2)cc1] | 5-(2-fluoro-6-hydroxy-3-(1-(4-methoxyphenyl)-1H- imidazol-4-yl)phenyl)-1,2,5-thiadiazolidin-3-one 1,1- dioxide | 417.0 |
| 349 | 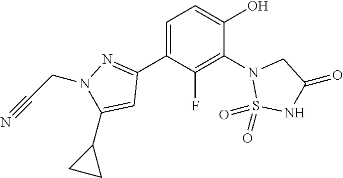 [SMILES: N#CCn1nc(-c2ccc(O)c(N3CC(=O)NS3(=O)=O)c2F)cc1C1CC1] | 2-(5-cyclopropyl-3-(3-(1,1-dioxido-4-oxo-1,2,5- thiadiazolidin-2-yl)-2-fluoro-4-hydroxyphenyl)-1H- pyrazol-1-yl)acetonitrile | 389.9 |
| 350 | 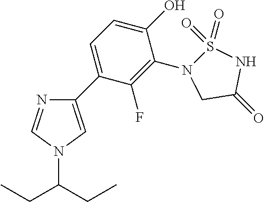 [SMILES: CCC(CC)n1cnc(-c2ccc(O)c(N3CC(=O)NS3(=O)=O)c2F)c1] | 5-(2-fluoro-6-hydroxy-3-(1-(pentan-3-yl)-1H- imidazol-4-yl)phenyl)-1,2,5-thiadiazolidin-3-one 1,1- dioxide | 381.0 |
| 351 | 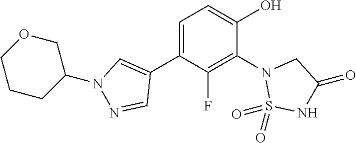 [SMILES: O=C1CN(c2c(O)ccc(-c3cnn(C4CCCOC4)c3)c2F)S(=O)(=O)N1] | 5-(2-fluoro-6-hydroxy-3-(1-(tetrahydro-2H-pyran-3- yl)-1H-pyrazol-4-yl)phenyl)-1,2,5-thiadiazolidin-3- one 1,1-dioxide | 395.2 |
| 352 | 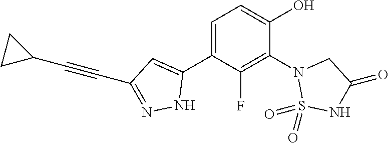 [SMILES: O=C1CN(c2c(O)ccc(-c3cc(C#CC4CC4)n[nH]3)c2F)S(=O)(=O)N1] | 5-(3-(3-(cyclopropylethynyl)-1H-pyrazol-5-yl)-2- fluoro-6-hydroxyphenyl)-1,2,5-thiadiazolidin-3-one 1,1-dioxide | 375.0 |
| 353 | 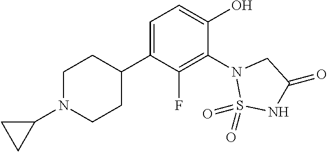 [SMILES: O=C1CN(c2c(O)ccc(C3CCN(C4CC4)CC3)c2F)S(=O)(=O)N1] | 5-(3-(1-cyclopropylpiperidin-4-yl)-2-fluoro-6- hydroxyphenyl)-1,2,5-thiadiazolidin-3-one 1,1- dioxide | 368.3 |
| 354 | 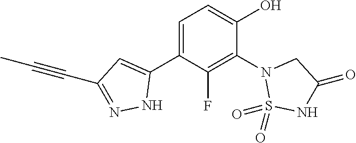 [SMILES: CC#Cc1cc(-c2ccc(O)c(N3CC(=O)NS3(=O)=O)c2F)[nH]n1] | 5-(2-fluoro-6-hydroxy-3-(3-(prop-1-yn-1-yl)-1H- pyrazol-5-yl)phenyl)-1,2,5-thiadiazolidin-3-one 1,1- dioxide | 348.8 |
| 355 | 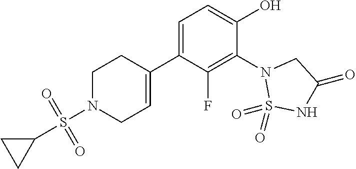 [SMILES: O=C1CN(c2c(O)ccc(C3=CCN(S(=O)(=O)C4CC4)CC3)c2F)S(=O)(=O)N1] | 5-(3-(1-(cyclopropylsulfonyl)-1,2,3,6- tetrahydropyridin-4-yl)-2-fluoro-6-hydroxyphenyl)- 1,2,5-thiadiazolidin-3-one 1,1-dioxide | 430.3 |
| 356 | 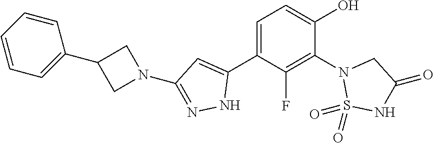 [SMILES: O=C1CN(c2c(O)ccc(-c3cc(N4CC(c5ccccc5)C4)n[nH]3)c2F)S(=O)(=O)N1] | 5-(2-fluoro-6-hydroxy-3-(3-(3-phenylazetidin-1-yl)- 1H-pyrazol-5-yl)phenyl)-1,2,5-thiadiazolidin-3-one 1,1-dioxide | 442.1 |
| 357 | 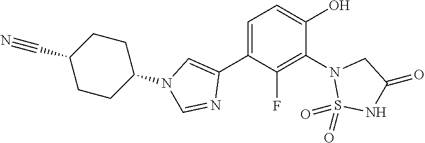 [SMILES: N#C[C@H]1CC[C@@H](n2cnc(-c3ccc(O)c(N4CC(=O)NS4(=O)=O)c3F)c2)CC1] | (1s,4s)-4-(4-(3-(1,1-dioxido-4-oxo-1,2,5- thiadiazolidin-2-yl)-2-fluoro-4-hydroxyphenyl)-1H- imidazol-1-yl)cyclohexane-1-carbonitrile | 418.3 |
| 358 | 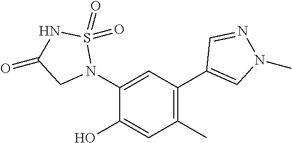 [SMILES: Cc1cc(O)c(N2CC(=O)NS2(=O)=O)cc1-c1cnn(C)c1] | 5-(2-hydroxy-4-methyl-5-(1-methyl-1H-pyrazol-4- yl)phenyl)-1,2,5-thiadiazolidin-3-one 1,1-dioxide | 321.2 |
| 359 | 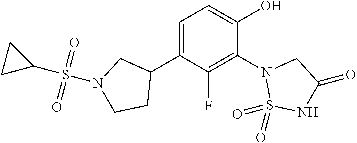 [SMILES: O=C1CN(c2c(O)ccc(C3CCN(S(=O)(=O)C4CC4)C3)c2F)S(=O)(=O)N1] | 5-(3-(1-(cyclopropylsulfonyl)pyrrolidin-3-yl)-2- fluoro-6-hydroxyphenyl)-1,2,5-thiadiazolidin-3-one 1,1-dioxide | 418.1 |
| 360 | 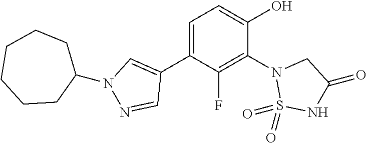 [SMILES: O=C1CN(c2c(O)ccc(-c3cnn(C4CCCCCC4)c3)c2F)S(=O)(=O)N1] | 5-(3-(1-cycloheptyl-1H-pyrazol-4-yl)-2-fluoro-6- hydroxyphenyl)-1,2,5-thiadiazolidin-3-one 1,1- dioxide | 407.0 |
| 361 | 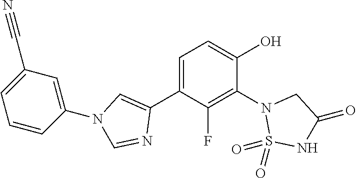 [SMILES: N#Cc1cccc(-n2cnc(-c3ccc(O)c(N4CC(=O)NS4(=O)=O)c3F)c2)c1] | 3-(4-(3-(1,1-dioxido-4-oxo-1,2,5-thiadiazolidin-2- yl)-2-fluoro-4-hydroxyphenyl)-1H-imidazol-1- yl)benzonitrile | 412.2 |
| 362 | 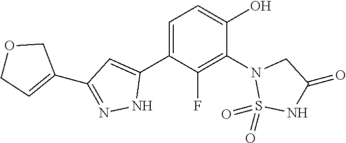 [SMILES: O=C1CN(c2c(O)ccc(-c3cc(C4=CCOC4)n[nH]3)c2F)S(=O)(=O)N1] | 5-(3-(3-(2,5-dihydrofuran-3-yl)-1H-pyrazol-5-yl)-2- fluoro-6-hydroxyphenyl)-1,2,5-thiadiazolidin-3-one 1,1-dioxide | 379.2 |
| 363 | 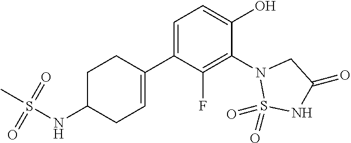 [SMILES: CS(=O)(=O)NC1CC=C(c2ccc(O)c(N3CC(=O)NS3(=O)=O)c2F)CC1] | N-(3′-(1,1-dioxido-4-oxo-1,2,5-thiadiazolidin-2-yl)- 2′-fluoro-4′-hydroxy-2,3,4,5-tetrahydro-[1,1′- biphenyl]-4-yl)methanesulfonamide | 418.0 |
| 364 | 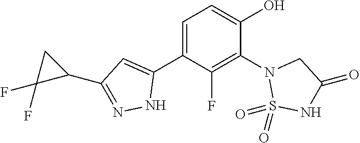 [SMILES: O=C1CN(c2c(O)ccc(-c3cc(C4CC4(F)F)n[nH]3)c2F)S(=O)(=O)N1] | 5-(3-(3-(2,2-difluorocyclopropyl)-1H-pyrazol-5-yl)- 2-fluoro-6-hydroxyphenyl)-1,2,5-thiadiazolidin-3- one 1,1-dioxide | 387.0 |
| 365 | 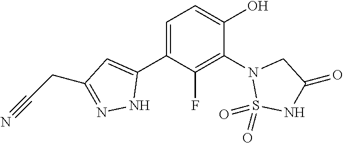 [SMILES: N#CCc1cc(-c2ccc(O)c(N3CC(=O)NS3(=O)=O)c2F)[nH]n1] | 2-(5-(3-(1,1-dioxido-4-oxo-1,2,5-thiadiazolidin-2- yl)-2-fluoro-4-hydroxyphenyl)-1H-pyrazol-3- yl)acetonitrile | 350.2 |
| 366 | 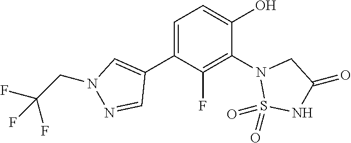 [SMILES: O=C1CN(c2c(O)ccc(-c3cnn(CC(F)(F)F)c3)c2F)S(=O)(=O)N1] | 5-(2-fluoro-6-hydroxy-3-(1-(2,2,2-trifluoroethyl)- 1H-pyrazol-4-yl)phenyl)-1,2,5-thiadiazolidin-3-one 1,1-dioxide | 393.0 |
| 367 | 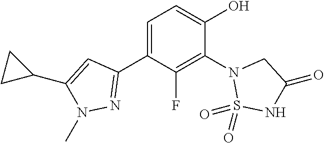 [SMILES: Cn1nc(-c2ccc(O)c(N3CC(=O)NS3(=O)=O)c2F)cc1C1CC1] | 5-(3-(5-cyclopropyl-1-methyl-1H-pyrazol-3-yl)-2- fluoro-6-hydroxyphenyl)-1,2,5-thiadiazolidin-3-one 1,1-dioxide | 365.2 |
| 368 | 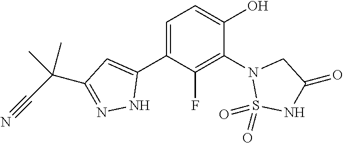 [SMILES: CC(C)(C#N)c1cc(-c2ccc(O)c(N3CC(=O)NS3(=O)=O)c2F)[nH]n1] | 2-(5-(3-(1,1-dioxido-4-oxo-1,2,5-thiadiazolidin-2- yl)-2-fluoro-4-hydroxyphenyl)-1H-pyrazol-3-yl)-2- methylpropanenitrile | 380.2 [M + H]⁺ |
| 369 | 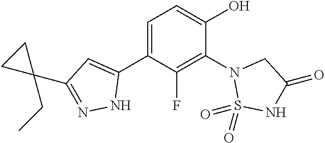 [SMILES: CCC1(c2cc(-c3ccc(O)c(N4CC(=O)NS4(=O)=O)c3F)[nH]n2)CC1] | 5-(3-(3-(1-ethylcyclopropyl)-1H-pyrazol-5-yl)-2- fluoro-6-hydroxyphenyl)-1,2,5-thiadiazolidin-3-one 1,1-dioxide | 379.0 |
| 370 | 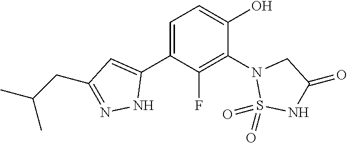 [SMILES: CC(C)Cc1cc(-c2ccc(O)c(N3CC(=O)NS3(=O)=O)c2F)[nH]n1] | 5-(2-fluoro-6-hydroxy-3-(3-isobutyl-1H-pyrazol-5- yl)phenyl)-1,2,5-thiadiazolidin-3-one 1,1-dioxide | 367.1 |
| 371 | 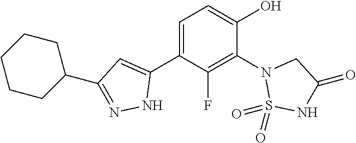 [SMILES: O=C1CN(c2c(O)ccc(-c3cc(C4CCCCC4)n[nH]3)c2F)S(=O)(=O)N1] | 5-(3-(3-cyclohexyl-1H-pyrazol-5-yl)-2-fluoro-6- hydroxyphenyl)-1,2,5-thiadiazolidin-3-one 1,1- dioxide | 392.9 |
| 372 | 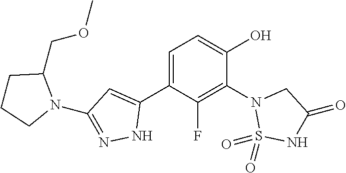 [SMILES: COCC1CCCN1c1cc(-c2ccc(O)c(N3CC(=O)NS3(=O)=O)c2F)[nH]n1] | 5-(2-fluoro-6-hydroxy-3-(3-(2- (methoxymethyl)pyrrolidin-1-yl)-1H-pyrazol-5- yl)phenyl)-1,2,5-thiadiazolidin-3-one 1,1-dioxide | 424.3 |
| 373 | 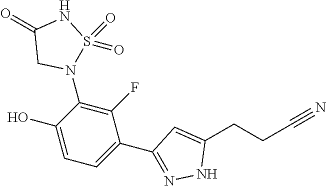 [SMILES: N#CCCc1cc(-c2ccc(O)c(N3CC(=O)NS3(=O)=O)c2F)n[nH]1] | 3-(3-(3-(1,1-dioxido-4-oxo-1,2,5-thiadiazolidin-2- yl)-2-fluoro-4-hydroxyphenyl)-1H-pyrazol-5- yl)propanenitrile | 364.0 |
| 374 | 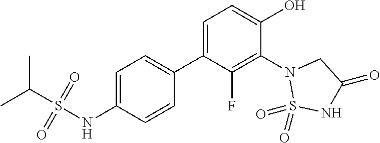 [SMILES: CC(C)S(=O)(=O)Nc1ccc(-c2ccc(O)c(N3CC(=O)NS3(=O)=O)c2F)cc1] | N-(3′-(1,1-dioxido-4-oxo-1,2,5-thiadiazolidin-2-yl)- 2′-fluoro-4′-hydroxy-[1,1′-biphenyl]-4-yl)propane-2- sulfonamide | 442.2 |
| 375 | 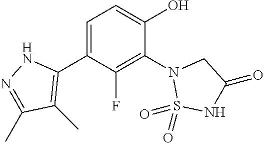 [SMILES: Cc1n[nH]c(-c2ccc(O)c(N3CC(=O)NS3(=O)=O)c2F)c1C] | 5-(3-(3,4-dimethyl-1H-pyrazol-5-yl)-2-fluoro-6- hydroxyphenyl)-1,2,5-thiadiazolidin-3-one 1,1- dioxide | 341.2 [M + H]⁺ |
| 376 | 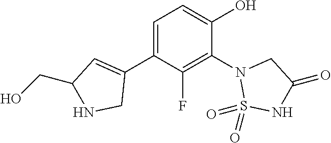 [SMILES: O=C1CN(c2c(O)ccc(C3=CC(CO)NC3)c2F)S(=O)(=O)N1] | 5-(3-(3-ethyl-1H-pyrazol-5-yl)-2-fluoro-6- hydroxyphenyl)-1,2,5-thiadiazolidin-3-one 1,1- dioxide | 339.0 |
| 377 | 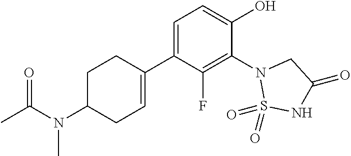 [SMILES: CC(=O)N(C)C1CC=C(c2ccc(O)c(N3CC(=O)NS3(=O)=O)c2F)CC1] | N-(3′-(1,1-dioxido-4-oxo-1,2,5-thiadiazolidin-2-yl)- 2′-fluoro-4′-hydroxy-2,3,4,5-tetrahydro-[1,1′- biphenyl]-4-yl)-N-methylacetamide | 396.2 |
| 378 | 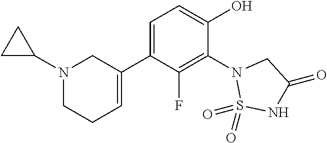 [SMILES: O=C1CN(c2c(O)ccc(C3=CCCN(C4CC4)C3)c2F)S(=O)(=O)N1] | 5-(3-(1-cyclopropyl-1,2,5,6-tetrahydropyridin-3-yl)- 2-fluoro-6-hydroxyphenyl)-1,2,5-thiadiazolidin-3- one 1,1-dioxide | 366.2 |
| 379 | 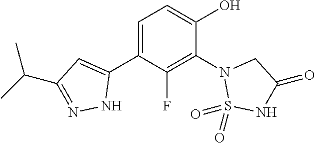 [SMILES: CC(C)c1cc(-c2ccc(O)c(N3CC(=O)NS3(=O)=O)c2F)[nH]n1] | 5-(2-fluoro-6-hydroxy-3-(3-isopropyl-1H-pyrazol-5- yl)phenyl)-1,2,5-thiadiazolidin-3-one 1,1-dioxide | 353.0 |
| 380 | 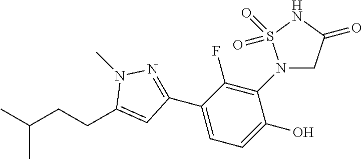 [SMILES: CC(C)CCc1cc(-c2ccc(O)c(N3CC(=O)NS3(=O)=O)c2F)nn1C] | 5-(2-fluoro-6-hydroxy-3-(5-isopentyl-1-methyl-1H- pyrazol-3-yl)phenyl)-1,2,5-thiadiazolidin-3-one 1,1- dioxide | 395.3 |
| 381 | 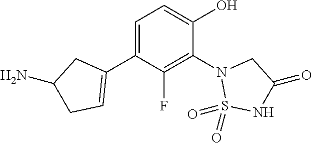 [SMILES: NC1CC=C(c2ccc(O)c(N3CC(=O)NS3(=O)=O)c2F)C1] | 5-(3-(4-aminocyclopent-1-en-1-yl)-2-fluoro-6- hydroxyphenyl)-1,2,5-thiadiazolidin-3-one 1,1- dioxide | 326.3 |
| 382 | 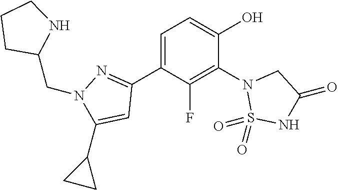 [SMILES: O=C1CN(c2c(O)ccc(-c3cc(C4CC4)n(CC4CCCN4)n3)c2F)S(=O)(=O)N1] | 5-(3-(5-cyclopropyl-1-(pyrrolidin-2-ylmethyl)-1H- pyrazol-3-yl)-2-fluoro-6-hydroxyphenyl)-1,2,5- thiadiazolidin-3-one 1,1-dioxide | 434.3 |
| 383 | 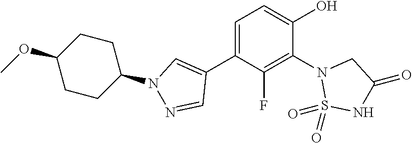 [SMILES: CO[C@H]1CC[C@@H](n2cc(-c3ccc(O)c(N4CC(=O)NS4(=O)=O)c3F)cn2)CC1] | 5-(2-fluoro-6-hydroxy-3-(1-((1s,4s)-4- methoxycyclohexyl)-1H-pyrazol-4-yl)phenyl)-1,2,5- thiadiazolidin-3-one 1,1-dioxide | 422.9 |
| 384 | 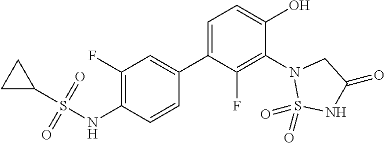 [SMILES: O=C1CN(c2c(O)ccc(-c3ccc(NS(=O)(=O)C4CC4)c(F)c3)c2F)S(=O)(=O)N1] | N-(3′-(1,1-dioxido-4-oxo-1,2,5-thiadiazolidin-2-yl)- 2′,3-difluoro-4′-hydroxy-[1,1′-biphenyl]-4- yl)cyclopropanesulfonamide | 458.1 |
| 385 | 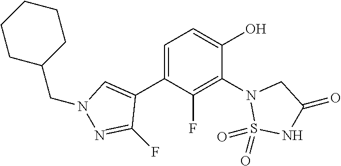 [SMILES: O=C1CN(c2c(O)ccc(-c3cn(CC4CCCCC4)nc3F)c2F)S(=O)(=O)N1] | 5-(3-(1-(cyclohexylmethyl)-3-fluoro-1H-pyrazol-4- yl)-2-fluoro-6-hydroxyphenyl)-1,2,5-thiadiazolidin- 3-one 1,1-dioxide | 427.1 [M + H]⁺ |
| 386 | 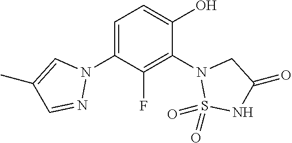 [SMILES: Cc1cnn(-c2ccc(O)c(N3CC(=O)NS3(=O)=O)c2F)c1] | 5-(2-fluoro-6-hydroxy-3-(4-methyl-1H-pyrazol-1- yl)phenyl)-1,2,5-thiadiazolidin-3-one 1,1-dioxide | 327.2 [M + H]⁺ |
| 387 | 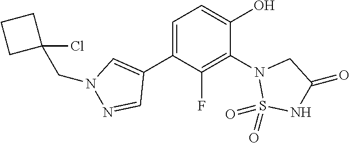 [SMILES: O=C1CN(c2c(O)ccc(-c3cnn(CC4(Cl)CCC4)c3)c2F)S(=O)(=O)N1] | 5-(3-(1-((1-chlorocyclobutyl)methyl)-1H-pyrazol-4- yl)-2-fluoro-6-hydroxyphenyl)-1,2,5-thiadiazolidin- 3-one 1,1-dioxide | 412.9 |
| 388 | 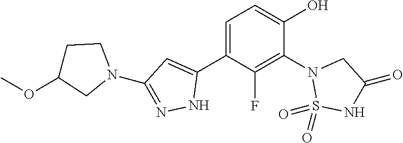 [SMILES: COC1CCN(c2cc(-c3ccc(O)c(N4CC(=O)NS4(=O)=O)c3F)[nH]n2)C1] | 5-(2-fluoro-6-hydroxy-3-(3-(3-methoxypyrrolidin-1- yl)-1H-pyrazol-5-yl)phenyl)-1,2,5-thiadiazolidin-3- one 1,1-dioxide | 410.2 |
| 389 | 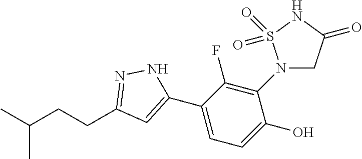 [SMILES: CC(C)CCc1cc(-c2ccc(O)c(N3CC(=O)NS3(=O)=O)c2F)[nH]n1] | 5-(2-fluoro-6-hydroxy-3-(3-isopentyl-1H-pyrazol-5- yl)phenyl)-1,2,5-thiadiazolidin-3-one 1,1-dioxide | 381.0 |
| 390 | 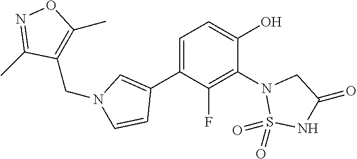 [SMILES: Cc1noc(C)c1Cn1ccc(-c2ccc(O)c(N3CC(=O)NS3(=O)=O)c2F)c1] | 5-(3-(1-((3,5-dimethylisoxazol-4-yl)methyl)-1H- pyrrol-3-yl)-2-fluoro-6-hydroxyphenyl)-1,2,5- thiadiazolidin-3-one 1,1-dioxide | 419.1 |
| 391 | 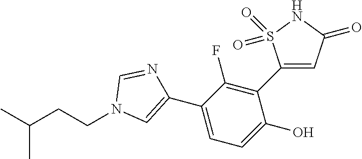 [SMILES: CC(C)CCn1cnc(-c2ccc(O)c(C3=CC(=O)NS3(=O)=O)c2F)c1] | 5-(2-fluoro-6-hydroxy-3-(1-isopentyl-1H-imidazol- 4-yl)phenyl)isothiazol-3(2H)-one 1,1-dioxide | 378.0 |
| 392 | 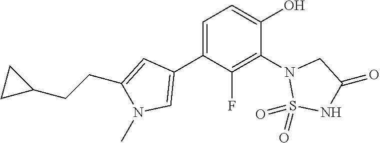 [SMILES: Cn1cc(-c2ccc(O)c(N3CC(=O)NS3(=O)=O)c2F)cc1CCC1CC1] | 5-(3-(5-(2-cyclopropylethyl)-1-methyl-1H-pyrrol-3- yl)-2-fluoro-6-hydroxyphenyl)-1,2,5-thiadiazolidin- 3-one 1,1-dioxide | 391.9 |
| 393 | 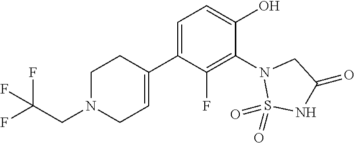 [SMILES: O=C1CN(c2c(O)ccc(C3=CCN(CC(F)(F)F)CC3)c2F)S(=O)(=O)N1] | 5-(2-fluoro-6-hydroxy-3-(1-(2,2,2-trifluoroethyl)- 1,2,3,6-tetrahydropyridin-4-yl)phenyl)-1,2,5- thiadiazolidin-3-one 1,1-dioxide | 408.2 |
| 394 | 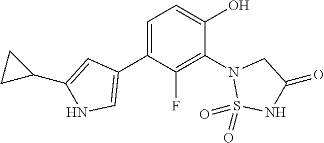 [SMILES: O=C1CN(c2c(O)ccc(-c3c[nH]c(C4CC4)c3)c2F)S(=O)(=O)N1] | 5-(3-(5-cyclopropyl-1H-pyrrol-3-yl)-2-fluoro-6- hydroxyphenyl)-1,2,5-thiadiazolidin-3-one 1,1- dioxide | 349.8 |
| 395 | 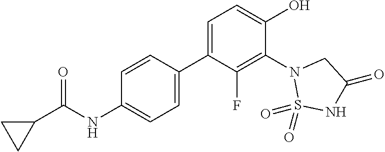 [SMILES: O=C1CN(c2c(O)ccc(-c3ccc(NC(=O)C4CC4)cc3)c2F)S(=O)(=O)N1] | N-(3′-(1,1-dioxido-4-oxo-1,2,5-thiadiazolidin-2-yl)- 2′-fluoro-4′-hydroxy-[1,1′-biphenyl]-4- yl)cyclopropanecarboxamide | 404.2 |
| 396 | 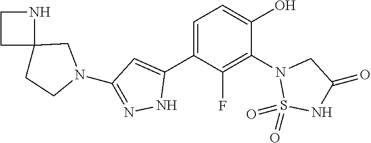 [SMILES: O=C1CN(c2c(O)ccc(-c3cc(N4CCC5(CCN5)C4)n[nH]3)c2F)S(=O)(=O)N1] | 5-(3-(3-(1,6-diazaspiro[3.4]octan-6-yl)-1H-pyrazol- 5-yl)-2-fluoro-6-hydroxyphenyl)-1,2,5- thiadiazolidin-3-one 1,1-dioxide | 421.0 |
| 397 | 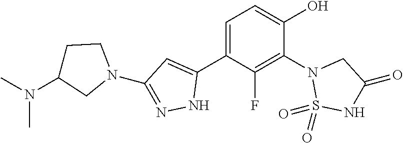 [SMILES: CN(C)C1CCN(c2cc(-c3ccc(O)c(N4CC(=O)NS4(=O)=O)c3F)[nH]n2)C1] | 5-(3-(3-(3-(dimethylamino)pyrrolidin-1-yl)-1H- pyrazol-5-yl)-2-fluoro-6-hydroxyphenyl)-1,2,5- thiadiazolidin-3-one 1,1-dioxide | 423.3 |
| 398 | 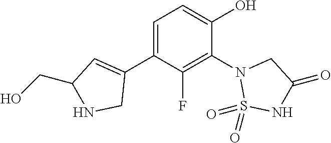 [SMILES: O=C1CN(c2c(O)ccc(C3=CC(CO)NC3)c2F)S(=O)(=O)N1] | 5-(2-fluoro-6-hydroxy-3-(5-(hydroxymethyl)-2,5- dihydro-1H-pyrrol-3-yl)phenyl)-1,2,5-thiadiazolidin- 3-one 1,1-dioxide | 342.0 |
| 399 | 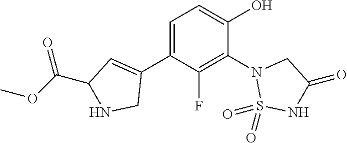 [SMILES: COC(=O)C1C=C(c2ccc(O)c(N3CC(=O)NS3(=O)=O)c2F)CN1] | methyl 4-(3-(1,1-dioxido-4-oxo-1,2,5-thiadiazolidin- 2-yl)-2-fluoro-4-hydroxyphenyl)-2,5-dihydro-1H- pyrrole-2-carboxylate | 369.9 |
| 400 | 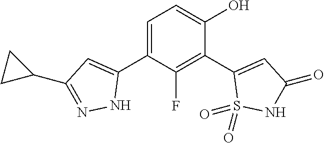 [SMILES: O=C1C=C(c2c(O)ccc(-c3cc(C4CC4)n[nH]3)c2F)S(=O)(=O)N1] | 5-(3-(3-cyclopropyl-1H-pyrazol-5-yl)-2-fluoro-6- hydroxyphenyl)isothiazol-3(2H)-one 1,1-dioxide | 348.2 |
| 401 | 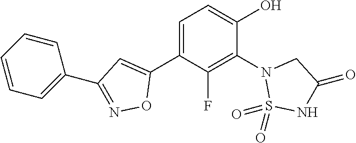 [SMILES: O=C1CN(c2c(O)ccc(-c3cc(-c4ccccc4)no3)c2F)S(=O)(=O)N1] | 5-(2-fluoro-6-hydroxy-3-(3-phenylisoxazol-5- yl)phenyl)-1,2,5-thiadiazolidin-3-one 1,1-dioxide | 388.1 |
| 402 | 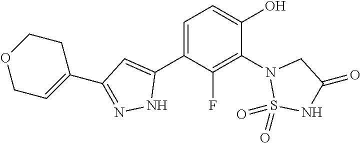 [SMILES: O=C1CN(c2c(O)ccc(-c3cc(C4=CCOCC4)n[nH]3)c2F)S(=O)(=O)N1] | 5-(3-(3-(3,6-dihydro-2H-pyran-4-yl)-1H-pyrazol-5- yl)-2-fluoro-6-hydroxyphenyl)-1,2,5-thiadiazolidin- 3-one 1,1-dioxide | 393.1 |
| 403 | 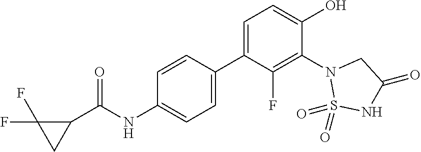 [SMILES: O=C1CN(c2c(O)ccc(-c3ccc(NC(=O)C4CC4(F)F)cc3)c2F)S(=O)(=O)N1] | N-(3′-(1,1-dioxido-4-oxo-1,2,5-thiadiazolidin-2-yl)- 2′-fluoro-4′-hydroxy-[1,1′-biphenyl]-4-yl)-2,2- difluorocyclopropane-1-carboxamide | 440.2 |
| 404 | 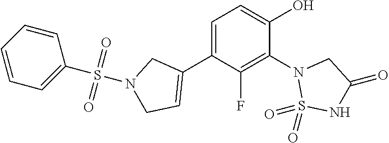 [SMILES: O=C1CN(c2c(O)ccc(C3=CCN(S(=O)(=O)c4ccccc4)C3)c2F)S(=O)(=O)N1] | 5-(2-fluoro-6-hydroxy-3-(1-(phenylsulfonyl)-2,5- dihydro-1H-pyrrol-3-yl)phenyl)-1,2,5-thiadiazolidin- 3-one 1,1-dioxide | 451.9 |
| 405 | 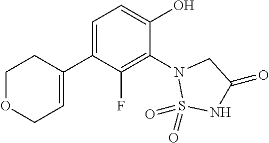 [SMILES: O=C1CN(c2c(O)ccc(C3=CCOCC3)c2F)S(=O)(=O)N1] | 5-(3-(3,6-dihydro-2H-pyran-4-yl)-2-fluoro-6- hydroxyphenyl)-1,2,5-thiadiazolidin-3-one 1,1- dioxide | 327.2 |
| 406 | 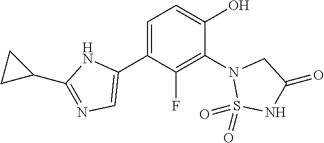 [SMILES: O=C1CN(c2c(O)ccc(-c3cnc(C4CC4)[nH]3)c2F)S(=O)(=O)N1] | 5-(3-(2-cyclopropyl-1H-imidazol-5-yl)-2-fluoro-6- hydroxyphenyl)-1,2,5-thiadiazolidin-3-one 1,1- dioxide | 351.0 |
| 407 | 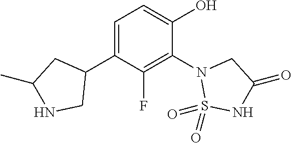 [SMILES: CC1CC(c2ccc(O)c(N3CC(=O)NS3(=O)=O)c2F)CN1] | 5-(2-fluoro-6-hydroxy-3-(5-methylpyrrolidin-3- yl)phenyl)-1,2,5-thiadiazolidin-3-one 1,1-dioxide | 328.0 |
| 408 | 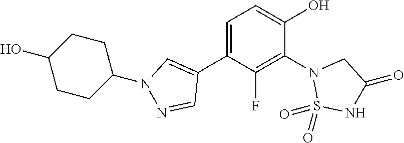 [SMILES: O=C1CN(c2c(O)ccc(-c3cnn(C4CCC(O)CC4)c3)c2F)S(=O)(=O)N1] | 5-(2-fluoro-6-hydroxy-3-(1-(4-hydroxycyclohexyl)- 1H-pyrazol-4-yl)phenyl)-1,2,5-thiadiazolidin-3-one 1,1-dioxide | 411.2 |
| 409 | 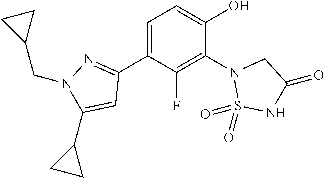 [SMILES: O=C1CN(c2c(O)ccc(-c3cc(C4CC4)n(CC4CC4)n3)c2F)S(=O)(=O)N1] | 5-(3-(5-cyclopropyl-1-(cyclopropylmethyl)-1H- pyrazol-3-yl)-2-fluoro-6-hydroxyphenyl)-1,2,5- thiadiazolidin-3-one 1,1-dioxide | 405.0 |
| 410 | 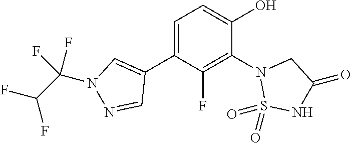 [SMILES: O=C1CN(c2c(O)ccc(-c3cnn(C(F)(F)C(F)F)c3)c2F)S(=O)(=O)N1] | 5-(2-fluoro-6-hydroxy-3-(1-(1,1,2,2- tetrafluoroethyl)-1H-pyrazol-4-yl)phenyl)-1,2,5- thiadiazolidin-3-one 1,1-dioxide | 411.0 |
| 411 | 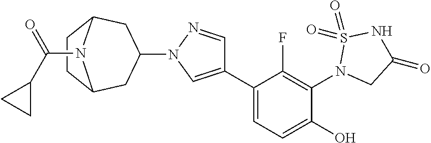 [SMILES: O=C1CN(c2c(O)ccc(-c3cnn(C4CC5CCC(C4)N5C(=O)C4CC4)c3)c2F)S(=O)(=O)N1] | 5-(3-(1-(8-(cyclopropanecarbonyl)-8- azabicyclo[3.2.1]octan-3-yl)-1H-pyrazol-4-yl)-2- fluoro-6-hydroxyphenyl)-1,2,5-thiadiazolidin-3-one 1,1-dioxide | 488.1 |
| 412 | 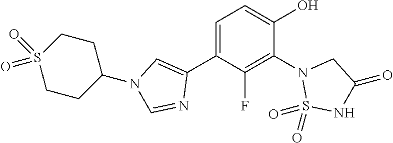 [SMILES: O=C1CN(c2c(O)ccc(-c3cn(C4CCS(=O)(=O)CC4)cn3)c2F)S(=O)(=O)N1] | 5-(3-(1-(1,1-dioxidotetrahydro-2H-thiopyran-4-yl)- 1H-imidazol-4-yl)-2-fluoro-6-hydroxyphenyl)-1,2,5- thiadiazolidin-3-one 1,1-dioxide | 442.9 |
| 413 | 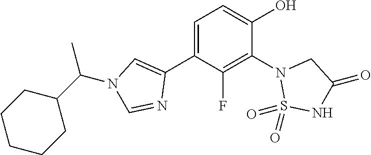 [SMILES: CC(C1CCCCC1)n1cnc(-c2ccc(O)c(N3CC(=O)NS3(=O)=O)c2F)c1] | 5-(3-(1-(1-cyclohexylethyl)-1H-imidazol-4-yl)-2- fluoro-6-hydroxyphenyl)-1,2,5-thiadiazolidin-3-one 1,1-dioxide | 421.3 |
| 414 | 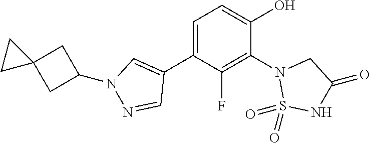 [SMILES: O=C1CN(c2c(O)ccc(-c3cnn(C4CC5(CC5)C4)c3)c2F)S(=O)(=O)N1] | 5-(2-fluoro-6-hydroxy-3-(1-(spiro[2.3]hexan-5-yl)- 1H-pyrazol-4-yl)phenyl)-1,2,5-thiadiazolidin-3-one 1,1-dioxide | 391.3 |
| 415 | 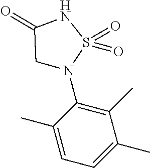 [SMILES: Cc1ccc(C)c(N2CC(=O)NS2(=O)=O)c1C] | 5-(2-fluoro-6-hydroxy-3-(3-(3,3,3-trifluoropropyl)- 1H-pyrazol-5-yl)phenyl)-1,2,5-thiadiazolidin-3-one 1,1-dioxide | 407.2 |
| 416 | 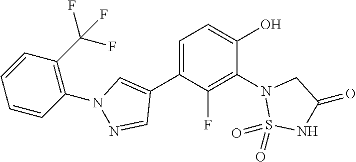 [SMILES: O=C1CN(c2c(O)ccc(-c3cnn(-c4ccccc4C(F)(F)F)c3)c2F)S(=O)(=O)N1] | 5-(2-fluoro-6-hydroxy-3-(1-(2- (trifluoromethyl)phenyl)-1H-imidazol-4-yl)phenyl)- 1,2,5-thiadiazolidin-3-one 1,1-dioxide | 457.1 [M + H]⁺ |
| 417 | 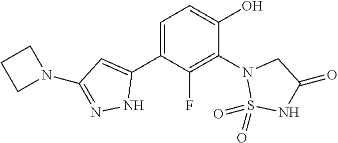 [SMILES: O=C1CN(c2c(O)ccc(-c3cc(N4CCC4)n[nH]3)c2F)S(=O)(=O)N1] | 5-(3-(3-(azetidin-1-yl)-1H-pyrazol-5-yl)-2-fluoro-6- hydroxyphenyl)-1,2,5-thiadiazolidin-3-one 1,1- dioxide | 366.1 |
| 418 | 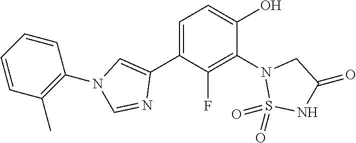 [SMILES: Cc1ccccc1-n1cnc(-c2ccc(O)c(N3CC(=O)NS3(=O)=O)c2F)c1] | 5-(2-fluoro-6-hydroxy-3-(1-(o-tolyl)-1H-imidazol-4- yl)phenyl)-1,2,5-thiadiazolidin-3-one 1,1-dioxide | 401.0 |
| 419 | 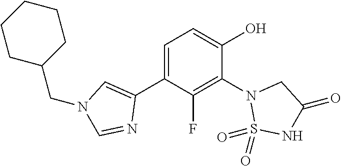 [SMILES: O=C1CN(c2c(O)ccc(-c3cn(CC4CCCCC4)cn3)c2F)S(=O)(=O)N1] | 5-(3-(1-(cyclohexylmethyl)-1H-imidazol-4-yl)-2- fluoro-6-hydroxyphenyl)isothiazolidin-3-one 1,1- dioxide | 406.3 |
| 420 | 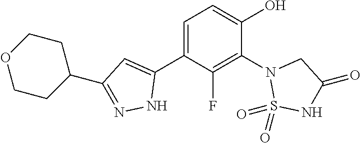 [SMILES: O=C1CN(c2c(O)ccc(-c3cc(C4CCOCC4)n[nH]3)c2F)S(=O)(=O)N1] | 5-(2-fluoro-6-hydroxy-3-(3-(tetrahydro-2H-pyran-4- yl)-1H-pyrazol-5-yl)phenyl)-1,2,5-thiadiazolidin-3- one 1,1-dioxide | 395.2 |
| 421 | 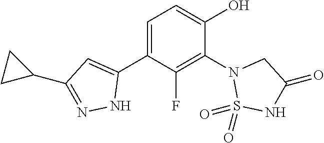 [SMILES: O=C1CN(c2c(O)ccc(-c3cc(C4CC4)n[nH]3)c2F)S(=O)(=O)N1] | 5-(3-(3-cyclopropyl-1H-pyrazol-5-yl)-2-fluoro-6- hydroxyphenyl)-1,2,5-thiadiazolidin-3-one 1,1- dioxide | 351.0 |
| 422 | 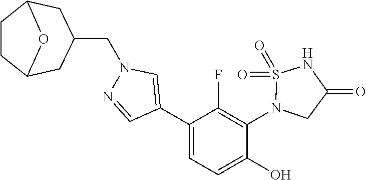 [SMILES: O=C1CN(c2c(O)ccc(-c3cnn(CC4CC5CCC(C4)O5)c3)c2F)S(=O)(=O)N1] | 5-(3-(1-((8-oxabicyclo[3.2.1]octan-3-yl)methyl)-1H- pyrazol-4-yl)-2-fluoro-6-hydroxyphenyl)-1,2,5- thiadiazolidin-3-one 1,1-dioxide | 435.0 |
| 423 | 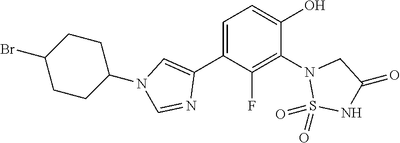 [SMILES: O=C1CN(c2c(O)ccc(-c3cn(C4CCC(Br)CC4)cn3)c2F)S(=O)(=O)N1] | 5-(3-(1-(4-bromocyclohexyl)-1H-imidazol-4-yl)-2- fluoro-6-hydroxyphenyl)-1,2,5-thiadiazolidin-3-one 1,1-dioxide | 472.9 |
| 424 | 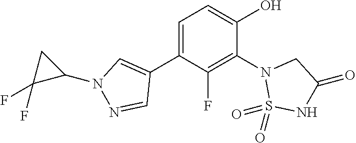 [SMILES: O=C1CN(c2c(O)ccc(-c3cnn(C4CC4(F)F)c3)c2F)S(=O)(=O)N1] | 5-(3-(1-(2,2-difluorocyclopropyl)-1H-pyrazol-4-yl)- 2-fluoro-6-hydroxyphenyl)-1,2,5-thiadiazolidin-3- one 1,1-dioxide | 386.8 |
| 425 | 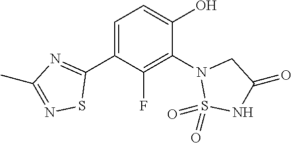 [SMILES: Cc1nsc(-c2ccc(O)c(N3CC(=O)NS3(=O)=O)c2F)n1] | 5-(2-fluoro-6-hydroxy-3-(3-methyl-1,2,4-thiadiazol- 5-yl)phenyl)-1,2,5-thiadiazolidin-3-one 1,1-dioxide | 343.1 |
| 426 | 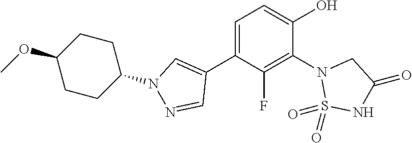 [SMILES: CO[C@H]1CC[C@H](n2cc(-c3ccc(O)c(N4CC(=O)NS4(=O)=O)c3F)cn2)CC1] | 5-(2-fluoro-6-hydroxy-3-(1-((1r,4r)-4- methoxycyclohexyl)-1H-pyrazol-4-yl)phenyl)-1,2,5- thiadiazolidin-3-one 1,1-dioxide | 422.9 |
| 427 | 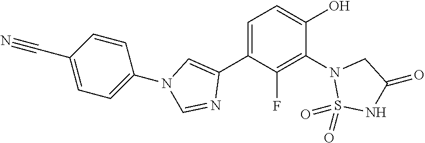 [SMILES: N#Cc1ccc(-n2cnc(-c3ccc(O)c(N4CC(=O)NS4(=O)=O)c3F)c2)cc1] | 4-(4-(3-(1,1-dioxido-4-oxo-1,2,5-thiadiazolidin-2- yl)-2-fluoro-4-hydroxyphenyl)-1H-imidazol-1- yl)benzonitrile | 412.0 |
| 428 | 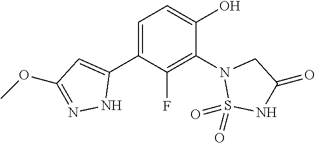 [SMILES: COc1cc(-c2ccc(O)c(N3CC(=O)NS3(=O)=O)c2F)[nH]n1] | 5-(2-fluoro-6-hydroxy-3-(3-methoxy-1H-pyrazol-5- yl)phenyl)-1,2,5-thiadiazolidin-3-one 1,1-dioxide | 340.9 |
| 429 | 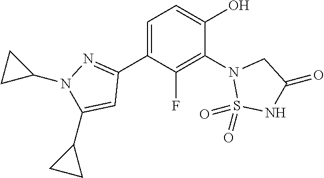 [SMILES: O=C1CN(c2c(O)ccc(-c3cc(C4CC4)n(C4CC4)n3)c2F)S(=O)(=O)N1] | 5-(3-(1,5-dicyclopropyl-1H-pyrazol-3-yl)-2-fluoro-6- hydroxyphenyl)-1,2,5-thiadiazolidin-3-one 1,1- dioxide | 391.2 |
| 430 | 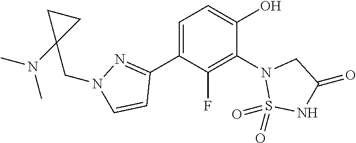 [SMILES: CN(C)C1(Cn2ccc(-c3ccc(O)c(N4CC(=O)NS4(=O)=O)c3F)n2)CC1] | 5-(3-(1-((1-(dimethylamino)cyclobutyl)methyl)-1H- pyrazol-4-yl)-2-fluoro-6-hydroxyphenyl)-1,2,5- thiadiazolidin-3-one 1,1-dioxide | 422.1 |
| 431 | 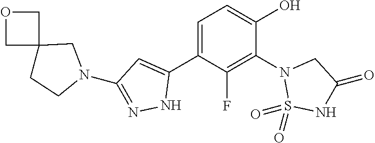 [SMILES: O=C1CN(c2c(O)ccc(-c3cc(N4CCC5(COC5)C4)n[nH]3)c2F)S(=O)(=O)N1] | 5-(3-(3-(2-oxa-6-azaspiro[3.4]octan-6-yl)-1H- pyrazol-5-yl)-2-fluoro-6-hydroxyphenyl)-1,2,5- thiadiazolidin-3-one 1,1-dioxide | 422.2 |
| 432 | 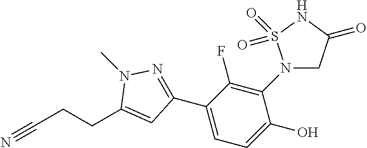 [SMILES: Cn1nc(-c2ccc(O)c(N3CC(=O)NS3(=O)=O)c2F)cc1CCC#N] | 3-(3-(3-(1,1-dioxido-4-oxo-1,2,5-thiadiazolidin-2- yl)-2-fluoro-4-hydroxyphenyl)-1-methyl-1H-pyrazol- 5-yl)propanenitrile | 377.9 |
| 433 | 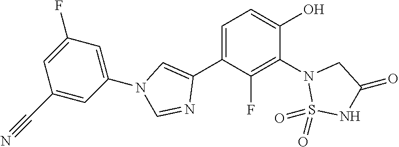 [SMILES: N#Cc1cc(F)cc(-n2cnc(-c3ccc(O)c(N4CC(=O)NS4(=O)=O)c3F)c2)c1] | 3-(4-(3-(1,1-dioxido-4-oxo-1,2,5-thiadiazolidin-2- yl)-2-fluoro-4-hydroxyphenyl)-1H-imidazol-1-yl)-5- fluorobenzonitrile | 430.0 |
| 434 | 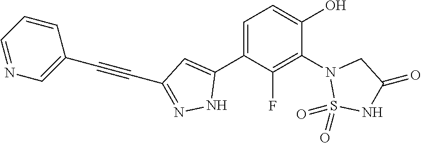 [SMILES: O=C1CN(c2c(O)ccc(-c3cc(C#Cc4cccnc4)n[nH]3)c2F)S(=O)(=O)N1] | 5-(2-fluoro-6-hydroxy-3-(3-(pyridin-3-ylethynyl)- 1H-pyrazol-5-yl)phenyl)-1,2,5-thiadiazolidin-3-one 1,1-dioxide | 412.0 |
| 435 | 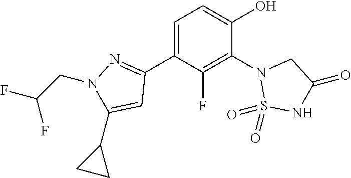 [SMILES: O=C1CN(c2c(O)ccc(-c3cc(C4CC4)n(CC(F)F)n3)c2F)S(=O)(=O)N1] | 5-(3-(5-cyclopropyl-1-(2,2-difluoroethyl)-1H- pyrazol-3-yl)-2-fluoro-6-hydroxyphenyl)-1,2,5- thiadiazolidin-3-one 1,1-dioxide | 415.2 |
| 436 | 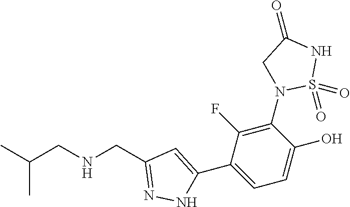 [SMILES: CC(C)CNCc1cc(-c2ccc(O)c(N3CC(=O)NS3(=O)=O)c2F)[nH]n1] | 5-(2-fluoro-6-hydroxy-3-(3-((isobutylamino)methyl)- 1H-pyrazol-5-yl)phenyl)-1,2,5-thiadiazolidin-3-one 1,1-dioxide | 396.2 |
| 437 | 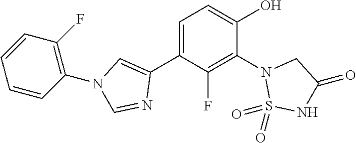 [SMILES: O=C1CN(c2c(O)ccc(-c3cn(-c4ccccc4F)cn3)c2F)S(=O)(=O)N1] | 5-(2-fluoro-3-(1-(2-fluorophenyl)-1H-imidazol-4-yl)- 6-hydroxyphenyl)-1,2,5-thiadiazolidin-3-one 1,1- dioxide | 405.2 |
| 438 | 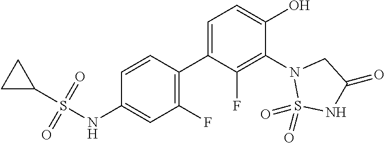 [SMILES: O=C1CN(c2c(O)ccc(-c3ccc(NS(=O)(=O)C4CC4)cc3F)c2F)S(=O)(=O)N1] | N-(3′-(1,1-dioxido-4-oxo-1,2,5-thiadiazolidin-2-yl)- 2,2′-difluoro-4′-hydroxy-[1,1′-biphenyl]-4- yl)cyclopropanesulfonamide | 458.1 |
| 439 | 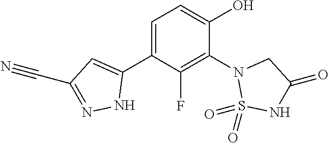 [SMILES: N#Cc1cc(-c2ccc(O)c(N3CC(=O)NS3(=O)=O)c2F)[nH]n1] | 5-(3-(1,1-dioxido-4-oxo-1,2,5-thiadiazolidin-2-yl)-2- fluoro-4-hydroxyphenyl)-1H-pyrazole-3-carbonitrile | 336.0 |
| 440 | 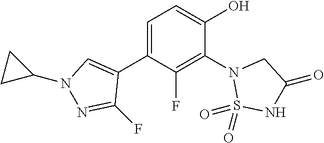 [SMILES: O=C1CN(c2c(O)ccc(-c3cn(C4CC4)nc3F)c2F)S(=O)(=O)N1] | 5-(3-(1-cyclopropyl-3-fluoro-1H-pyrazol-4-yl)-2- fluoro-6-hydroxyphenyl)-1,2,5-thiadiazolidin-3-one 1,1-dioxide | 369.2 |
| 441 | 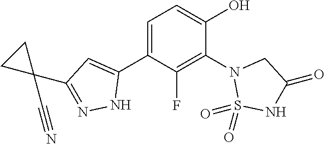 [SMILES: N#CC1(c2cc(-c3ccc(O)c(N4CC(=O)NS4(=O)=O)c3F)[nH]n2)CC1] | 1-(5-(3-(1,1-dioxido-4-oxo-1,2,5-thiadiazolidin-2- yl)-2-fluoro-4-hydroxyphenyl)-1H-pyrazol-3- yl)cyclopropane-1-carbonitrile | 378.2 [M + H]⁺ |
| 442 | 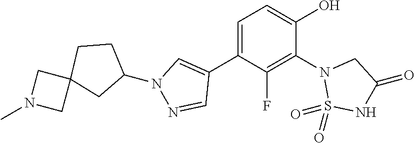 [SMILES: CN1CC2(CCC(n3cc(-c4ccc(O)c(N5CC(=O)NS5(=O)=O)c4F)cn3)C2)C1] | 5-(2-fluoro-6-hydroxy-3-(1-(2-methyl-2- azaspiro[3.4]octan-6-yl)-1H-pyrazol-4-yl)phenyl)- 1,2,5-thiadiazolidin-3-one 1,1-dioxide | 434.2 |
| 443 | 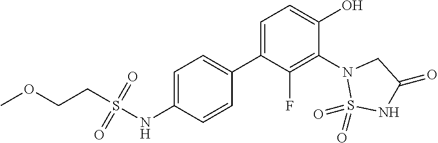 [SMILES: COCCS(=O)(=O)Nc1ccc(-c2ccc(O)c(N3CC(=O)NS3(=O)=O)c2F)cc1] | N-(3′-(1,1-dioxido-4-oxo-1,2,5-thiadiazolidin-2-yl)- 2′-fluoro-4′-hydroxy-[1,1′-biphenyl]-4-yl)-2- methoxyethane-1-sulfonamide | 458.2 |
| 444 | 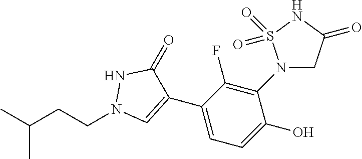 [SMILES: CC(C)CCn1cc(-c2ccc(O)c(N3CC(=O)NS3(=O)=O)c2F)c(=O)[nH]1] | 5-(2-fluoro-6-hydroxy-3-(1-isopentyl-3-oxo-2,3- dihydro-1H-pyrazol-4-yl)phenyl)-1,2,5- thiadiazolidin-3-one 1,1-dioxide | 397.0 |
| 445 | 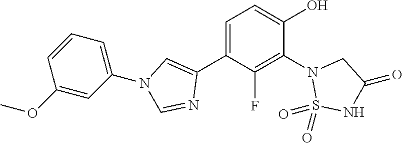 [SMILES: COc1cccc(-n2cnc(-c3ccc(O)c(N4CC(=O)NS4(=O)=O)c3F)c2)c1] | 5-(2-fluoro-6-hydroxy-3-(1-(3-methoxyphenyl)-1H- imidazol-4-yl)phenyl)-1,2,5-thiadiazolidin-3-one 1,1- dioxide | 417.2 |
| 446 | 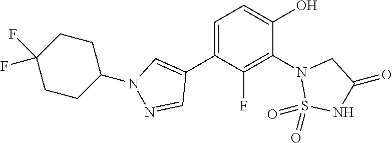 [SMILES: O=C1CN(c2c(O)ccc(-c3cnn(C4CCC(F)(F)CC4)c3)c2F)S(=O)(=O)N1] | 5-(3-(1-(4,4-difluorocyclohexyl)-1H-pyrazol-4-yl)-2- fluoro-6-hydroxyphenyl)-1,2,5-thiadiazolidin-3-one 1,1-dioxide | 429.0 |
| 447 | 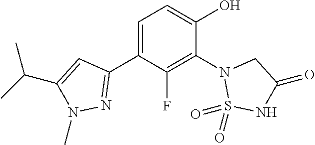 [SMILES: CC(C)c1cc(-c2ccc(O)c(N3CC(=O)NS3(=O)=O)c2F)nn1C] | 5-(2-fluoro-6-hydroxy-3-(5-isopropyl-1-methyl-1H- pyrazol-3-yl)phenyl)-1,2,5-thiadiazolidin-3-one 1,1- dioxide | 367.1 |
| 448 | 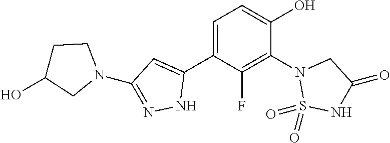 [SMILES: O=C1CN(c2c(O)ccc(-c3cc(N4CCC(O)C4)n[nH]3)c2F)S(=O)(=O)N1] | 5-(2-fluoro-6-hydroxy-3-(3-(3-hydroxypyrrolidin-1- yl)-1H-pyrazol-5-yl)phenyl)-1,2,5-thiadiazolidin-3- one 1,1-dioxide | 396.0 |
| 449 | 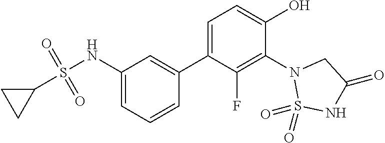 [SMILES: O=C1CN(c2c(O)ccc(-c3cccc(NS(=O)(=O)C4CC4)c3)c2F)S(=O)(=O)N1] | N-(3′-(1,1-dioxido-4-oxo-1,2,5-thiadiazolidin-2-yl)- 2′-fluoro-4′-hydroxy-[1,1′-biphenyl]-3- yl)cyclopropanesulfonamide | 440.2 |
| 450 | 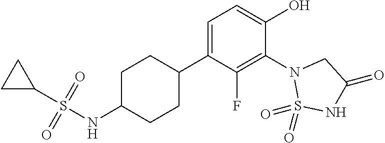 [SMILES: O=C1CN(c2c(O)ccc(C3CCC(NS(=O)(=O)C4CC4)CC3)c2F)S(=O)(=O)N1] | N-(4-(3-(1,1-dioxido-4-oxo-1,2,5-thiadiazolidin-2- yl)-2-fluoro-4- hydroxyphenyl)cyclohexyl)cyclopropanesulfonamide | 446.2 |
| 451 | 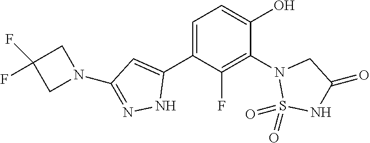 [SMILES: O=C1CN(c2c(O)ccc(-c3cc(N4CC(F)(F)C4)n[nH]3)c2F)S(=O)(=O)N1] | 5-(3-(3-(3,3-difluoroazetidin-1-yl)-1H-pyrazol-5-yl)- 2-fluoro-6-hydroxyphenyl)-1,2,5-thiadiazolidin-3- one 1,1-dioxide | 402.0 |
| 452 | 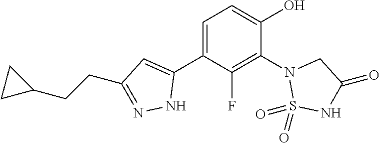 [SMILES: O=C1CN(c2c(O)ccc(-c3cc(CCC4CC4)n[nH]3)c2F)S(=O)(=O)N1] | 5-(3-(3-(2-cyclopropylethyl)-1H-pyrazol-5-yl)-2- fluoro-6-hydroxyphenyl)-1,2,5-thiadiazolidin-3-one 1,1-dioxide | 379.1 |
| 453 | 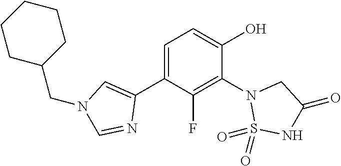 [SMILES: O=C1CN(c2c(O)ccc(-c3cn(CC4CCCCC4)cn3)c2F)S(=O)(=O)N1] | 5-(3-(1-(cyclohexylmethyl)-1H-imidazol-4-yl)-2- fluoro-6-hydroxyphenyl)-1,2,5-thiadiazolidin-3-one 1,1-dioxide | 407.0 [M + H]⁺ |
| 454 | 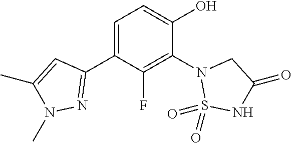 [SMILES: Cc1cc(-c2ccc(O)c(N3CC(=O)NS3(=O)=O)c2F)nn1C] | 5-(3-(1,5-dimethyl-1H-pyrazol-3-yl)-2-fluoro-6- hydroxyphenyl)-1,2,5-thiadiazolidin-3-one 1,1- dioxide | 339.2 |
| 455 | 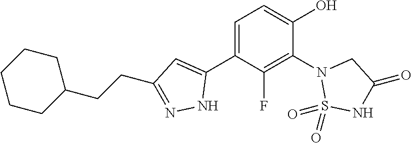 [SMILES: O=C1CN(c2c(O)ccc(-c3cc(CCC4CCCCC4)n[nH]3)c2F)S(=O)(=O)N1] | 5-(3-(3-(2-cyclohexylethyl)-1H-pyrazol-5-yl)-2- fluoro-6-hydroxyphenyl)-1,2,5-thiadiazolidin-3-one 1,1-dioxide | 421.3 |
| 456 | 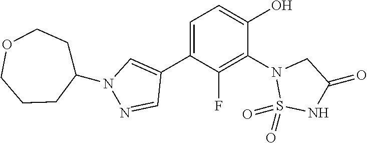 [SMILES: O=C1CN(c2c(O)ccc(-c3cnn(C4CCCOCC4)c3)c2F)S(=O)(=O)N1] | 5-(2-fluoro-6-hydroxy-3-(1-(oxepan-4-yl)-1H- pyrazol-4-yl)phenyl)-1,2,5-thiadiazolidin-3-one 1,1- dioxide | 409.3 |
| 457 | 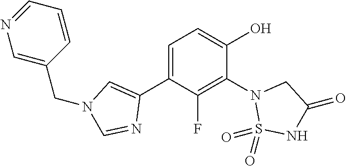 [SMILES: O=C1CN(c2c(O)ccc(-c3cn(Cc4cccnc4)cn3)c2F)S(=O)(=O)N1] | 5-(2-fluoro-6-hydroxy-3-(1-(pyridin-3-ylmethyl)-1H- imidazol-4-yl)phenyl)-1,2,5-thiadiazolidin-3-one 1,1- dioxide | 402.2 |
| 458 | 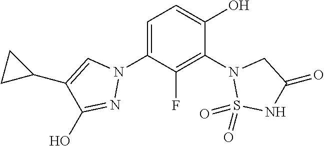 [SMILES: O=C1CN(c2c(O)ccc(-n3cc(C4CC4)c(O)n3)c2F)S(=O)(=O)N1] | 5-(3-(4-cyclopropyl-3-hydroxy-1H-pyrazol-1-yl)-2- fluoro-6-hydroxyphenyl)-1,2,5-thiadiazolidin-3-one 1,1-dioxide | 367.0 |
| 459 | 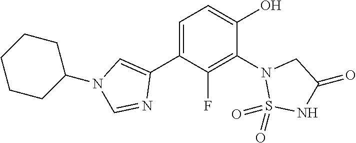 [SMILES: O=C1CN(c2c(O)ccc(-c3cn(C4CCCCC4)cn3)c2F)S(=O)(=O)N1] | 5-(3-(1-cyclohexyl-1H-imidazol-4-yl)-2-fluoro-6- hydroxyphenyl)-1,2,5-thiadiazolidin-3-one 1,1- dioxide | 393.0 |
| 460 | 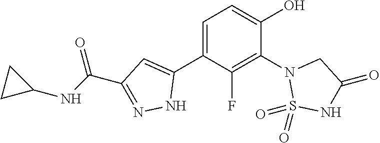 [SMILES: O=C1CN(c2c(O)ccc(-c3cc(C(=O)NC4CC4)n[nH]3)c2F)S(=O)(=O)N1] | N-cyclopropyl-5-(3-(1,1-dioxido-4-oxo-1,2,5- thiadiazolidin-2-yl)-2-fluoro-4-hydroxyphenyl)-1H- pyrazole-3-carboxamide | 394.1 |
| 461 | 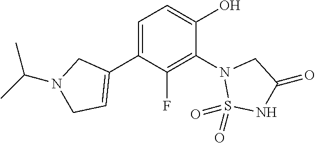 [SMILES: CC(C)N1CC=C(c2ccc(O)c(N3CC(=O)NS3(=O)=O)c2F)C1] | 5-(2-fluoro-6-hydroxy-3-(1-isopropyl-2,5-dihydro- 1H-pyrrol-3-yl)phenyl)-1,2,5-thiadiazolidin-3-one 1,1-dioxide | 353.9 |
| 462 | 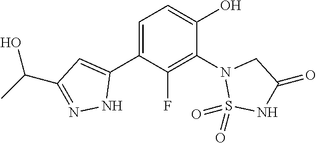 [SMILES: CC(O)c1cc(-c2ccc(O)c(N3CC(=O)NS3(=O)=O)c2F)[nH]n1] | 5-(2-fluoro-6-hydroxy-3-(3-(1-hydroxyethyl)-1H- pyrazol-5-yl)phenyl)-1,2,5-thiadiazolidin-3-one 1,1- dioxide | 355.2 |
| 463 | 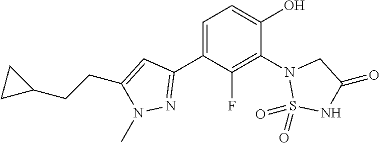 [SMILES: Cn1nc(-c2ccc(O)c(N3CC(=O)NS3(=O)=O)c2F)cc1CCC1CC1] | 5-(3-(5-(2-cyclopropylethyl)-1-methyl-1H-pyrazol-3- yl)-2-fluoro-6-hydroxyphenyl)-1,2,5-thiadiazolidin- 3-one 1,1-dioxide | 392.9 |
| 464 | 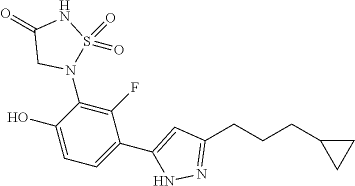 [SMILES: O=C1CN(c2c(O)ccc(-c3cc(CCCC4CC4)n[nH]3)c2F)S(=O)(=O)N1] | 5-(3-(3-(3-cyclopropylpropyl)-1H-pyrazol-5-yl)-2- fluoro-6-hydroxyphenyl)-1,2,5-thiadiazolidin-3-one 1,1-dioxide | 393.1 |
| 465 | 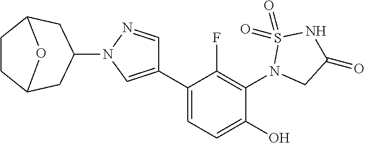 [SMILES: O=C1CN(c2c(O)ccc(-c3cnn(C4CC5CCC(C4)O5)c3)c2F)S(=O)(=O)N1] | 5-(3-(1-(8-oxabicyclo[3.2.1]octan-3-yl)-1H-pyrazol- 4-yl)-2-fluoro-6-hydroxyphenyl)-1,2,5- thiadiazolidin-3-one 1,1-dioxide | 421.2 |
| 466 | 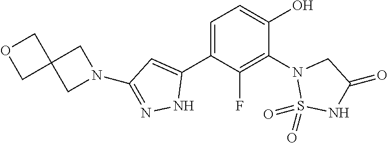 [SMILES: O=C1CN(c2c(O)ccc(-c3cc(N4CC5(COC5)C4)n[nH]3)c2F)S(=O)(=O)N1] | 5-(3-(3-(2-oxa-6-azaspiro[3.3]heptan-6-yl)-1H- pyrazol-5-yl)-2-fluoro-6-hydroxyphenyl)-1,2,5- thiadiazolidin-3-one 1,1-dioxide | 408.0 |
| 467 | 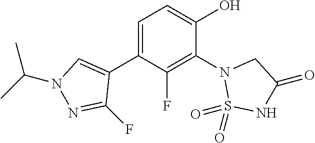 [SMILES: CC(C)n1cc(-c2ccc(O)c(N3CC(=O)NS3(=O)=O)c2F)c(F)n1] | 5-(2-fluoro-3-(3-fluoro-1-isopropyl-1H-pyrazol-4- yl)-6-hydroxyphenyl)-1,2,5-thiadiazolidin-3-one 1,1- dioxide | 371.0 |
| 468 | 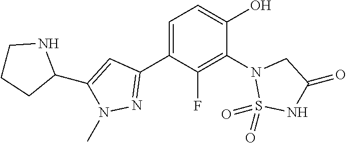 [SMILES: Cn1nc(-c2ccc(O)c(N3CC(=O)NS3(=O)=O)c2F)cc1C1CCCN1] | 5-(2-fluoro-6-hydroxy-3-(1-methyl-5-(pyrrolidin-2- yl)-1H-pyrazol-3-yl)phenyl)-1,2,5-thiadiazolidin-3- one 1,1-dioxide | 394.0 |
| 469 | 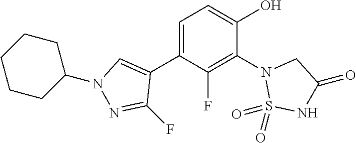 [SMILES: O=C1CN(c2c(O)ccc(-c3cn(C4CCCCC4)nc3F)c2F)S(=O)(=O)N1] | 5-(3-(1-cyclohexyl-3-fluoro-1H-pyrazol-4-yl)-2- fluoro-6-hydroxyphenyl)-1,2,5-thiadiazolidin-3-one 1,1-dioxide | 411.1 |
| 470 | 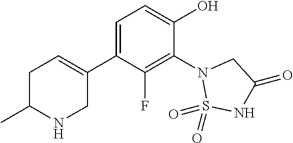 [SMILES: CC1CC=C(c2ccc(O)c(N3CC(=O)NS3(=O)=O)c2F)CN1] | 5-(2-fluoro-6-hydroxy-3-(6-methyl-1,2,5,6- tetrahydropyridin-3-yl)phenyl)-1,2,5-thiadiazolidin- 3-one 1,1-dioxide | 342.1 [M + H]⁺ |
| 471 | 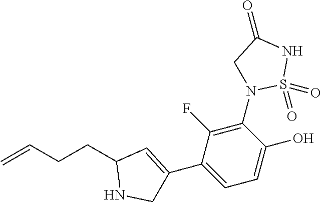 [SMILES: C=CCCC1C=C(c2ccc(O)c(N3CC(=O)NS3(=O)=O)c2F)CN1] | 5-(3-(5-(but-3-en-1-yl)-2,5-dihydro-1H-pyrrol-3-yl)- 2-fluoro-6-hydroxyphenyl)-1,2,5-thiadiazolidin-3- one 1,1-dioxide | 366.0 |
| 472 | 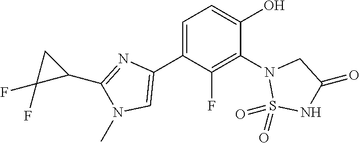 [SMILES: Cn1cc(-c2ccc(O)c(N3CC(=O)NS3(=O)=O)c2F)nc1C1CC1(F)F] | 5-(3-(2-(2,2-difluorocyclopropyl)-1-methyl-1H- imidazol-4-yl)-2-fluoro-6-hydroxyphenyl)-1,2,5- thiadiazolidin-3-one 1,1-dioxide | 401.1 |
| 473 | 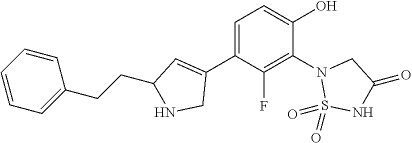 [SMILES: O=C1CN(c2c(O)ccc(C3=CC(CCc4ccccc4)NC3)c2F)S(=O)(=O)N1] | 5-(2-fluoro-6-hydroxy-3-(5-phenethyl-2,5-dihydro- 1H-pyrrol-3-yl)phenyl)-1,2,5-thiadiazolidin-3-one 1,1-dioxide | 416.1 |
| 474 | 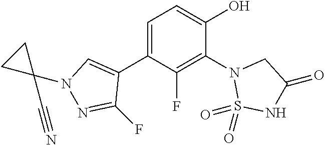 [SMILES: N#CC1(n2cc(-c3ccc(O)c(N4CC(=O)NS4(=O)=O)c3F)c(F)n2)CC1] | 1-(4-(3-(1,1-dioxido-4-oxo-1,2,5-thiadiazolidin-2- yl)-2-fluoro-4-hydroxyphenyl)-3-fluoro-1H-pyrazol- 1-yl)cyclopropane-1-carbonitrile | 394.2 |
| 475 | 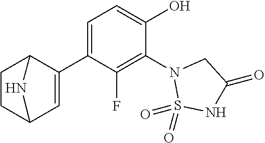 [SMILES: O=C1CN(c2c(O)ccc(C3=CC4CCC3N4)c2F)S(=O)(=O)N1] | 5-(3-(7-azabicyclo[2.2.1]hept-2-en-2-yl)-2-fluoro-6- hydroxyphenyl)-1,2,5-thiadiazolidin-3-one 1,1- dioxide | 338.0 |
| 476 | 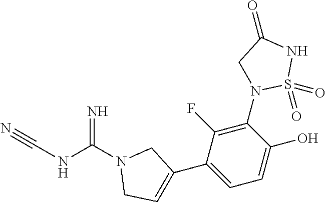 [SMILES: N#CNC(=N)N1CC=C(c2ccc(O)c(N3CC(=O)NS3(=O)=O)c2F)C1] | N-cyano-3-(3-(1,1-dioxido-4-oxo-1,2,5- thiadiazolidin-2-yl)-2-fluoro-4-hydroxyphenyl)-2,5- dihydro-1H-pyrrole-1-carboximidamide | 379.2 |
| 477 | 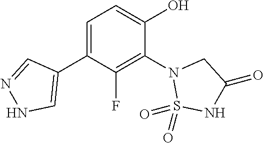 [SMILES: O=C1CN(c2c(O)ccc(-c3cn[nH]c3)c2F)S(=O)(=O)N1] | 5-(2-fluoro-6-hydroxy-3-(1H-pyrazol-4-yl)phenyl)- 1,2,5-thiadiazolidin-3-one 1,1-dioxide | 310.9 |
| 478 | 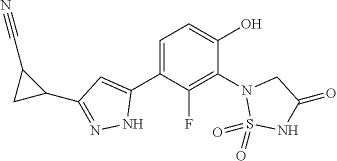 [SMILES: N#CC1CC1c1cc(-c2ccc(O)c(N3CC(=O)NS3(=O)=O)c2F)[nH]n1] | 2-(5-(3-(1,1-dioxido-4-oxo-1,2,5-thiadiazolidin-2- yl)-2-fluoro-4-hydroxyphenyl)-1H-pyrazol-3- yl)cyclopropane-1-carbonitrile | 376.3 |
| 479 | 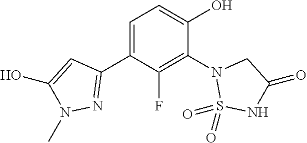 [SMILES: Cn1nc(-c2ccc(O)c(N3CC(=O)NS3(=O)=O)c2F)cc1O] | 5-(2-fluoro-6-hydroxy-3-(5-hydroxy-1-methyl-1H- pyrazol-3-yl)phenyl)-1,2,5-thiadiazolidin-3-one 1,1- dioxide | 341.1 |
| 480 | 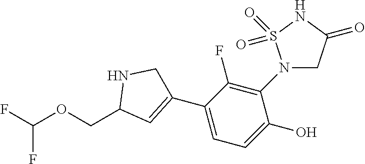 [SMILES: O=C1CN(c2c(O)ccc(C3=CC(COC(F)F)NC3)c2F)S(=O)(=O)N1] | 5-(3-(5-((difluoromethoxy)methyl)-2,5-dihydro-1H- pyrrol-3-yl)-2-fluoro-6-hydroxyphenyl)-1,2,5- thiadiazolidin-3-one 1,1-dioxide | 394.1 [M + H]⁺ |
| 481 | 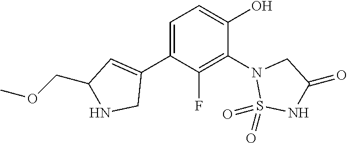 [SMILES: COCC1C=C(c2ccc(O)c(N3CC(=O)NS3(=O)=O)c2F)CN1] | 5-(2-fluoro-6-hydroxy-3-(5-(methoxymethyl)-2,5- dihydro-1H-pyrrol-3-yl)phenyl)-1,2,5-thiadiazolidin- 3-one 1,1-dioxide | 356.0 |
| 482 | 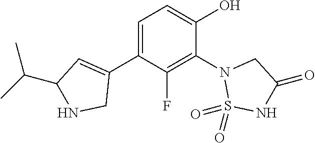 [SMILES: CC(C)C1C=C(c2ccc(O)c(N3CC(=O)NS3(=O)=O)c2F)CN1] | 5-(2-fluoro-6-hydroxy-3-(5-isopropyl-2,5-dihydro- 1H-pyrrol-3-yl)phenyl)-1,2,5-thiadiazolidin-3-one 1,1-dioxide | 354.1 |
| 483 | 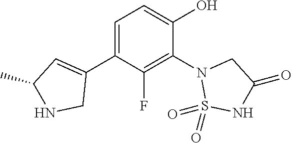 [SMILES: C[C@@H]1C=C(c2ccc(O)c(N3CC(=O)NS3(=O)=O)c2F)CN1] | (R)-5-(2-fluoro-6-hydroxy-3-(5-methyl-2,5-dihydro- 1H-pyrrol-3-yl)phenyl)-1,2,5-thiadiazolidin-3-one 1,1-dioxide | 328.1 [M + H]⁺ |
| 484 | 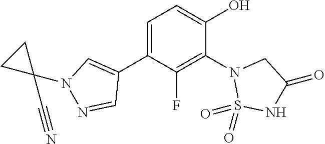 [SMILES: N#CC1(n2cc(-c3ccc(O)c(N4CC(=O)NS4(=O)=O)c3F)cn2)CC1] | 1-(4-(3-(1,1-dioxido-4-oxo-1,2,5-thiadiazolidin-2- yl)-2-fluoro-4-hydroxyphenyl)-1H-pyrazol-1- yl)cyclopropane-1-carbonitrile | 376.0 |
| 485 | 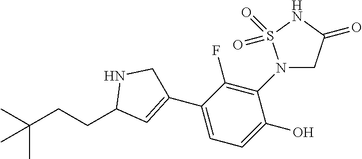 [SMILES: CC(C)(C)CCC1C=C(c2ccc(O)c(N3CC(=O)NS3(=O)=O)c2F)CN1] | 5-(3-(5-(3,3-dimethylbutyl)-2,5-dihydro-1H-pyrrol- 3-yl)-2-fluoro-6-hydroxyphenyl)-1,2,5- thiadiazolidin-3-one 1,1-dioxide | 396.3 |
| 486 | 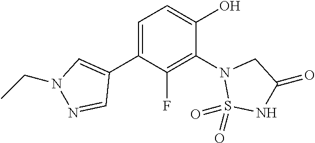 [SMILES: CCn1cc(-c2ccc(O)c(N3CC(=O)NS3(=O)=O)c2F)cn1] | 5-(3-(1-ethyl-1H-pyrazol-4-yl)-2-fluoro-6- hydroxyphenyl)-1,2,5-thiadiazolidin-3-one 1,1- dioxide | 339.0 |
| 487 | 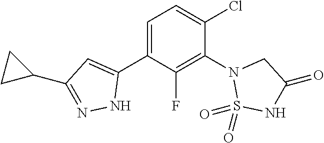 [SMILES: O=C1CN(c2c(Cl)ccc(-c3cc(C4CC4)n[nH]3)c2F)S(=O)(=O)N1] | 5-(6-chloro-3-(3-cyclopropyl-1H-pyrazol-5-yl)-2- fluorophenyl)-1,2,5-thiadiazolidin-3-one 1,1-dioxide | 369.0 |
| 488 | 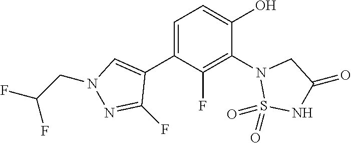 [SMILES: O=C1CN(c2c(O)ccc(-c3cn(CC(F)F)nc3F)c2F)S(=O)(=O)N1] | 5-(3-(1-(2,2-difluoroethyl)-3-fluoro-1H-pyrazol-4- yl)-2-fluoro-6-hydroxyphenyl)-1,2,5-thiadiazolidin- 3-one 1,1-dioxide | 393.0 |
| 489 | 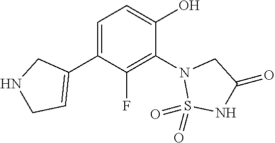 [SMILES: O=C1CN(c2c(O)ccc(C3=CCNC3)c2F)S(=O)(=O)N1] | 5-(3-(2,5-dihydro-1H-pyrrol-3-yl)-2-fluoro-6- hydroxyphenyl)-1,2,5-thiadiazolidin-3-one 1,1- dioxide | 312.0 |
| 490 | 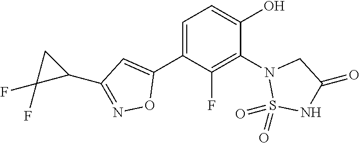 [SMILES: O=C1CN(c2c(O)ccc(-c3cc(C4CC4(F)F)no3)c2F)S(=O)(=O)N1] | 5-(3-(3-(2,2-difluorocyclopropyl)isoxazol-5-yl)-2- fluoro-6-hydroxyphenyl)-1,2,5-thiadiazolidin-3-one 1,1-dioxide | 388.1 |
| 491^{†} | 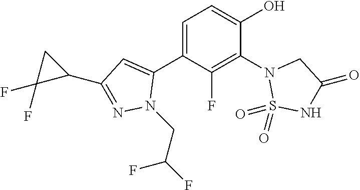 [SMILES: O=C1CN(c2c(O)ccc(-c3cc(C4CC4(F)F)nn3CC(F)F)c2F)S(=O)(=O)N1] | 5-(3-(3-(2,2-difluorocyclopropyl)-1-(2,2- difluoroethyl)-1H-pyrazol-5-yl)-2-fluoro-6- hydroxyphenyl)-1,2,5-thiadiazolidin-3-one 1,1- dioxide | 453.2 [M + H]⁺ |
| 492 | 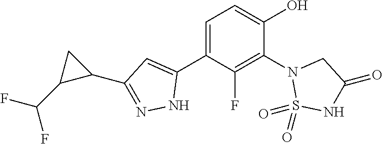 [SMILES: O=C1CN(c2c(O)ccc(-c3cc(C4CC4C(F)F)n[nH]3)c2F)S(=O)(=O)N1] | 5-(3-(3-(2-(difluoromethyl)cyclopropyl)-1H-pyrazol- 5-yl)-2-fluoro-6-hydroxyphenyl)-1,2,5- thiadiazolidin-3-one 1,1-dioxide | 401.3 |
| 493 | 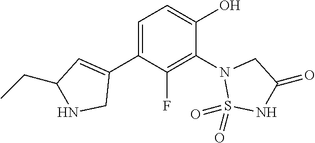 [SMILES: CCC1C=C(c2ccc(O)c(N3CC(=O)NS3(=O)=O)c2F)CN1] | 5-(3-(5-ethyl-2,5-dihydro-1H-pyrrol-3-yl)-2-fluoro- 6-hydroxyphenyl)-1,2,5-thiadiazolidin-3-one 1,1- dioxide | 340.1 |
| 494 | 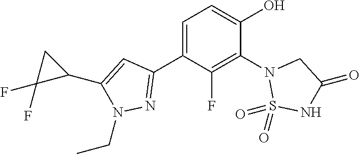 [SMILES: CCn1nc(-c2ccc(O)c(N3CC(=O)NS3(=O)=O)c2F)cc1C1CC1(F)F] | 5-(3-(5-(2,2-difluorocyclopropyl)-1-ethyl-1H- pyrazol-3-yl)-2-fluoro-6-hydroxyphenyl)-1,2,5- thiadiazolidin-3-one 1,1-dioxide | 415.0 |
| 495^{†} | 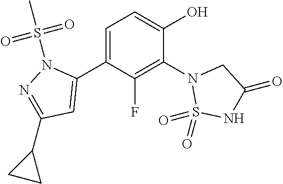 [SMILES: CS(=O)(=O)n1nc(C2CC2)cc1-c1ccc(O)c(N2CC(=O)NS2(=O)=O)c1F] | 5-(3-(3-cyclopropyl-1-(methylsulfonyl)-1H-pyrazol- 5-yl)-2-fluoro-6-hydroxyphenyl)-1,2,5- thiadiazolidin-3-one 1,1-dioxide | 429.0 |
| 496 | 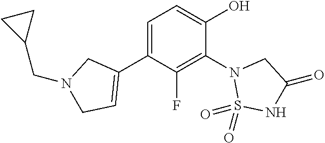 [SMILES: O=C1CN(c2c(O)ccc(C3=CCN(CC4CC4)C3)c2F)S(=O)(=O)N1] | 5-(3-(1-(cyclopropylmethyl)-2,5-dihydro-1H-pyrrol- 3-yl)-2-fluoro-6-hydroxyphenyl)-1,2,5- thiadiazolidin-3-one 1,1-dioxide | 366.3 |
| 497 | 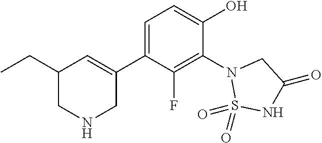 [SMILES: CCC1C=C(c2ccc(O)c(N3CC(=O)NS3(=O)=O)c2F)CNC1] | 5-(3-(5-ethyl-1,2,5,6-tetrahydropyridin-3-yl)-2- fluoro-6-hydroxyphenyl)-1,2,5-thiadiazolidin-3-one 1,1-dioxide | 354.2 |
| 498^{†} | 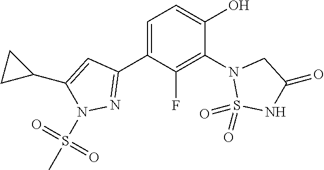 [SMILES: CS(=O)(=O)n1nc(-c2ccc(O)c(N3CC(=O)NS3(=O)=O)c2F)cc1C1CC1] | 5-(3-(5-cyclopropyl-1-(methylsulfonyl)-1H-pyrazol- 3-yl)-2-fluoro-6-hydroxyphenyl)-1,2,5- thiadiazolidin-3-one 1,1-dioxide | 429.0 |
| 499 | 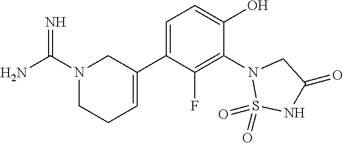 [SMILES: N=C(N)N1CCC=C(c2ccc(O)c(N3CC(=O)NS3(=O)=O)c2F)C1] | 5-(3-(1,1-dioxido-4-oxo-1,2,5-thiadiazolidin-2-yl)-2- fluoro-4-hydroxyphenyl)-3,6-dihydropyridine-1(2H)- carboximidamide | 368.1 |
| 500 | 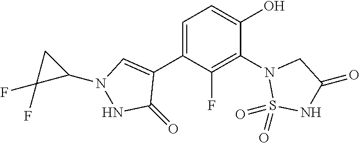 [SMILES: O=C1CN(c2c(O)ccc(-c3cn(C4CC4(F)F)[nH]c3=O)c2F)S(=O)(=O)N1] | 5-(3-(1-(2,2-difluorocyclopropyl)-3-oxo-2,3-dihydro- 1H-pyrazol-4-yl)-2-fluoro-6-hydroxyphenyl)-1,2,5- thiadiazolidin-3-one 1,1-dioxide | 403.0 |
| 501 | 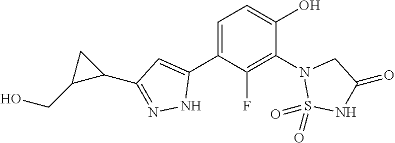 [SMILES: O=C1CN(c2c(O)ccc(-c3cc(C4CC4CO)n[nH]3)c2F)S(=O)(=O)N1] | 5-(2-fluoro-6-hydroxy-3-(3-(2- (hydroxymethyl)cyclopropyl)-1H-pyrazol-5- yl)phenyl)-1,2,5-thiadiazolidin-3-one 1,1-dioxide | 381.2 |
| 502 | 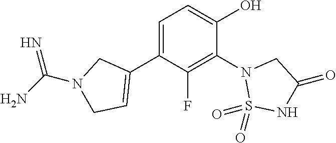 [SMILES: N=C(N)N1CC=C(c2ccc(O)c(N3CC(=O)NS3(=O)=O)c2F)C1] | 3-(3-(1,1-dioxido-4-oxo-1,2,5-thiadiazolidin-2-yl)-2- fluoro-4-hydroxyphenyl)-2,5-dihydro-1H-pyrrole-1- carboximidamide | 354.0 |
| 503 | 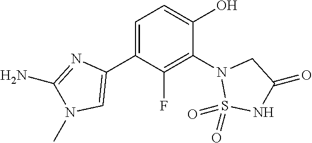 [SMILES: Cn1cc(-c2ccc(O)c(N3CC(=O)NS3(=O)=O)c2F)nc1N] | 5-(3-(2-amino-1-methyl-1H-imidazol-4-yl)-2-fluoro- 6-hydroxyphenyl)-1,2,5-thiadiazolidin-3-one 1,1- dioxide | 340.0 |
| 504 | 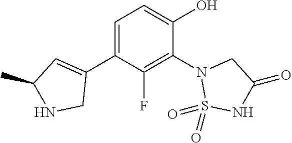 [SMILES: C[C@H]1C=C(c2ccc(O)c(N3CC(=O)NS3(=O)=O)c2F)CN1] | (S)-5-(2-fluoro-6-hydroxy-3-(5-methyl-2,5-dihydro- 1H-pyrrol-3-yl)phenyl)-1,2,5-thiadiazolidin-3-one 1,1-dioxide | 328.1 [M + H]⁺ |
| 505 | 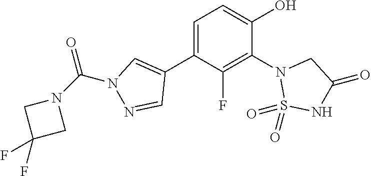 [SMILES: O=C1CN(c2c(O)ccc(-c3cnn(C(=O)N4CC(F)(F)C4)c3)c2F)S(=O)(=O)N1] | 5-(3-(1-(3,3-difluoroazetidine-1-carbonyl)-1H- pyrazol-4-yl)-2-fluoro-6-hydroxyphenyl)-1,2,5- thiadiazolidin-3-one 1,1-dioxide | 430.2 |
| 506 | 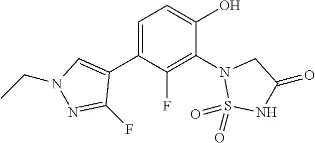 [SMILES: CCn1cc(-c2ccc(O)c(N3CC(=O)NS3(=O)=O)c2F)c(F)n1] | 5-(3-(1-ethyl-3-fluoro-1H-pyrazol-4-yl)-2-fluoro-6- hydroxyphenyl)-1,2,5-thiadiazolidin-3-one 1,1- dioxide | 357.0 |
| 507 | 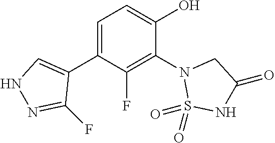 [SMILES: O=C1CN(c2c(O)ccc(-c3c[nH]nc3F)c2F)S(=O)(=O)N1] | 5-(2-fluoro-3-(3-fluoro-1H-pyrazol-4-yl)-6- hydroxyphenyl)-1,2,5-thiadiazolidin-3-one 1,1- dioxide | 329.2 |
| 508^{†} | 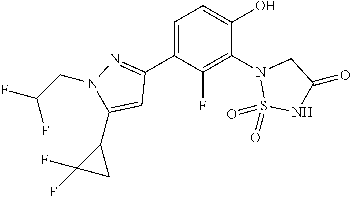 [SMILES: O=C1CN(c2c(O)ccc(-c3cc(C4CC4(F)F)n(CC(F)F)n3)c2F)S(=O)(=O)N1] | 5-(3-(5-(2,2-difluorocyclopropyl)-1-(2,2- difluoroethyl)-1H-pyrazol-3-yl)-2-fluoro-6- hydroxyphenyl)-1,2,5-thiadiazolidin-3-one 1,1- dioxide | 453.2 [M + H]⁺ |
| 509 | 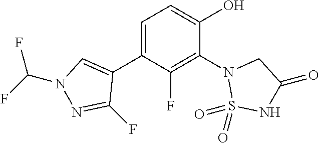 [SMILES: O=C1CN(c2c(O)ccc(-c3cn(C(F)F)nc3F)c2F)S(=O)(=O)N1] | 5-(3-(1-(difluoromethyl)-3-fluoro-1H-pyrazol-4-yl)- 2-fluoro-6-hydroxyphenyl)-1,2,5-thiadiazolidin-3- one 1,1-dioxide | 379.0 |
| 510 | 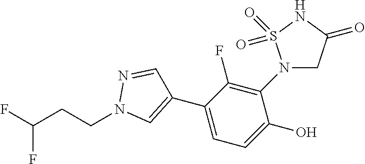 [SMILES: O=C1CN(c2c(O)ccc(-c3cnn(CCC(F)F)c3)c2F)S(=O)(=O)N1] | 5-(3-(1-(3,3-difluoropropyl)-1H-pyrazol-4-yl)-2- fluoro-6-hydroxyphenyl)-1,2,5-thiadiazolidin-3-one 1,1-dioxide | 389.0 |
| 511 | 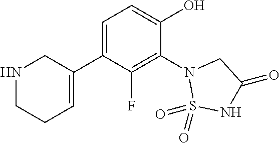 [SMILES: O=C1CN(c2c(O)ccc(C3=CCCNC3)c2F)S(=O)(=O)N1] | 5-(2-fluoro-6-hydroxy-3-(1,2,5,6-tetrahydropyridin- 3-yl)phenyl)-1,2,5-thiadiazolidin-3-one 1,1-dioxide | 326.0 |
| 512 | 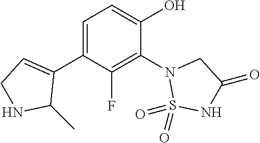 [SMILES: CC1NCC=C1c1ccc(O)c(N2CC(=O)NS2(=O)=O)c1F] | 5-(2-fluoro-6-hydroxy-3-(2-methyl-2,5-dihydro-1H- pyrrol-3-yl)phenyl)-1,2,5-thiadiazolidin-3-one 1,1- dioxide | 326.0 |
| 513 | 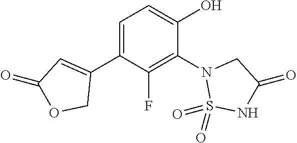 [SMILES: O=C1CN(c2c(O)ccc(C3=CC(=O)OC3)c2F)S(=O)(=O)N1] | 5-(2-fluoro-6-hydroxy-3-(5-oxo-2,5-dihydrofuran-3- yl)phenyl)-1,2,5-thiadiazolidin-3-one 1,1-dioxide | 327.2 |
| 514 | 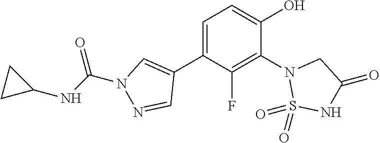 [SMILES: O=C1CN(c2c(O)ccc(-c3cnn(C(=O)NC4CC4)c3)c2F)S(=O)(=O)N1] | N-cyclopropyl-4-(3-(1,1-dioxido-4-oxo-1,2,5- thiadiazolidin-2-yl)-2-fluoro-4-hydroxyphenyl)-1H- pyrazole-1-carboxamide | 394.2 |
| 515 | 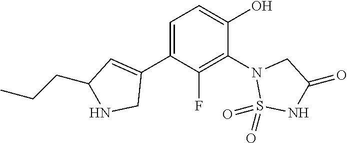 [SMILES: CCCC1C=C(c2ccc(O)c(N3CC(=O)NS3(=O)=O)c2F)CN1] | 5-(2-fluoro-6-hydroxy-3-(5-propyl-2,5-dihydro-1H- pyrrol-3-yl)phenyl)-1,2,5-thiadiazolidin-3-one 1,1- dioxide | 354.1 |
| 516 | 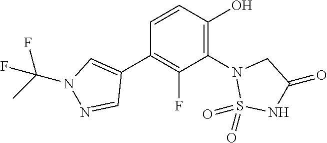 [SMILES: CC(F)(F)n1cc(-c2ccc(O)c(N3CC(=O)NS3(=O)=O)c2F)cn1] | 5-(3-(1-(1,1-difluoroethyl)-1H-pyrazol-4-yl)-2- fluoro-6-hydroxyphenyl)-1,2,5-thiadiazolidin-3-one 1,1-dioxide | 375.0 |
| 517 | 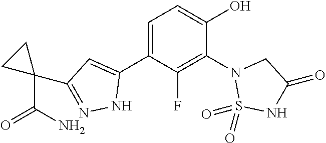 [SMILES: NC(=O)C1(c2cc(-c3ccc(O)c(N4CC(=O)NS4(=O)=O)c3F)[nH]n2)CC1] | 1-(5-(3-(1,1-dioxido-4-oxo-1,2,5-thiadiazolidin-2- yl)-2-fluoro-4-hydroxyphenyl)-1H-pyrazol-3- yl)cyclopropane-1-carboxamide | 394.2 |
| 518 | 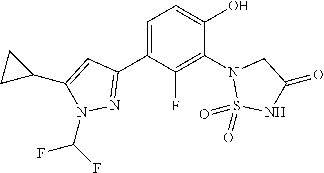 [SMILES: O=C1CN(c2c(O)ccc(-c3cc(C4CC4)n(C(F)F)n3)c2F)S(=O)(=O)N1] | 5-(3-(5-cyclopropyl-1-(difluoromethyl)-1H-pyrazol- 3-yl)-2-fluoro-6-hydroxyphenyl)-1,2,5- thiadiazolidin-3-one 1,1-dioxide | 401.0 |
| 519 | 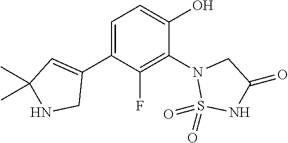 [SMILES: CC1(C)C=C(c2ccc(O)c(N3CC(=O)NS3(=O)=O)c2F)CN1] | 5-(3-(5,5-dimethyl-2,5-dihydro-1H-pyrrol-3-yl)-2- fluoro-6-hydroxyphenyl)-1,2,5-thiadiazolidin-3-one 1,1-dioxide | 342.2 [M + H]⁺ |
| 520 | 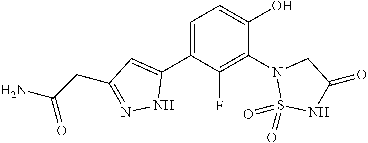 [SMILES: NC(=O)Cc1cc(-c2ccc(O)c(N3CC(=O)NS3(=O)=O)c2F)[nH]n1] | 2-(5-(3-(1,1-dioxido-4-oxo-1,2,5-thiadiazolidin-2- yl)-2-fluoro-4-hydroxyphenyl)-1H-pyrazol-3- yl)acetamide | 368.2 |
| 521 | 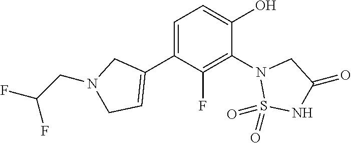 [SMILES: O=C1CN(c2c(O)ccc(C3=CCN(CC(F)F)C3)c2F)S(=O)(=O)N1] | 5-(3-(1-(2,2-difluoroethyl)-2,5-dihydro-1H-pyrrol-3- yl)-2-fluoro-6-hydroxyphenyl)-1,2,5-thiadiazolidin- 3-one 1,1-dioxide | 376.2 |
| 522 | 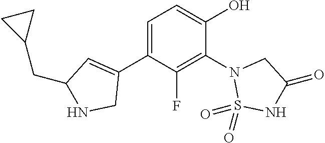 [SMILES: O=C1CN(c2c(O)ccc(C3=CC(CC4CC4)NC3)c2F)S(=O)(=O)N1] | 5-(3-(5-(cyclopropylmethyl)-2,5-dihydro-1H-pyrrol- 3-yl)-2-fluoro-6-hydroxyphenyl)-1,2,5- thiadiazolidin-3-one 1,1-dioxide | 366.0 |
| 523 | 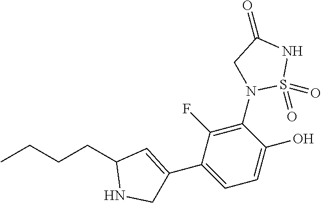 [SMILES: CCCCC1C=C(c2ccc(O)c(N3CC(=O)NS3(=O)=O)c2F)CN1] | 5-(3-(5-butyl-2,5-dihydro-1H-pyrrol-3-yl)-2-fluoro- 6-hydroxyphenyl)-1,2,5-thiadiazolidin-3-one 1,1- dioxide | 368.1 |
| 524 | 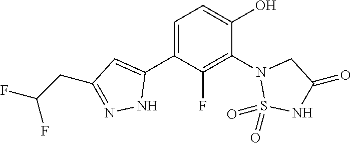 [SMILES: O=C1CN(c2c(O)ccc(-c3cc(CC(F)F)n[nH]3)c2F)S(=O)(=O)N1] | 5-(3-(3-(2,2-difluoroethyl)-1H-pyrazol-5-yl)-2- fluoro-6-hydroxyphenyl)-1,2,5-thiadiazolidin-3-one 1,1-dioxide | 375.0 |
| 525 | 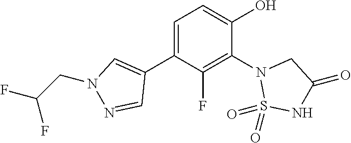 [SMILES: O=C1CN(c2c(O)ccc(-c3cnn(CC(F)F)c3)c2F)S(=O)(=O)N1] | 5-(3-(1-(2,2-difluoroethyl)-1H-pyrazol-4-yl)-2- fluoro-6-hydroxyphenyl)-1,2,5-thiadiazolidin-3-one 1,1-dioxide | 374.9 |
| 526 | 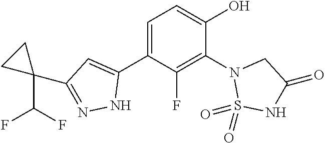 [SMILES: O=C1CN(c2c(O)ccc(-c3cc(C4(C(F)F)CC4)n[nH]3)c2F)S(=O)(=O)N1] | 5-(3-(3-(1-(difluoromethyl)cyclopropyl)-1H-pyrazol- 5-yl)-2-fluoro-6-hydroxyphenyl)-1,2,5- thiadiazolidin-3-one 1,1-dioxide | 401.0 |
| 527 | 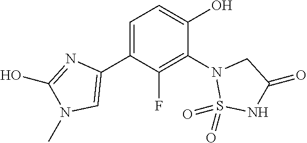 [SMILES: Cn1cc(-c2ccc(O)c(N3CC(=O)NS3(=O)=O)c2F)nc1O] | 5-(2-fluoro-6-hydroxy-3-(2-hydroxy-1-methyl-1H- imidazol-4-yl)phenyl)-1,2,5-thiadiazolidin-3-one 1,1- dioxide | 341.1 |
| 528^{†} | 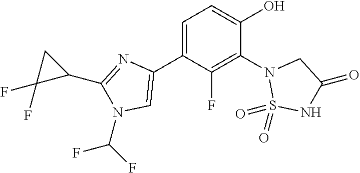 [SMILES: O=C1CN(c2c(O)ccc(-c3cn(C(F)F)c(C4CC4(F)F)n3)c2F)S(=O)(=O)N1] | 5-(3-(5-(2,2-difluorocyclopropyl)-1- (difluoromethyl)-1H-pyrazol-3-yl)-2-fluoro-6- hydroxyphenyl)-1,2,5-thiadiazolidin-3-one 1,1- dioxide | 439.1 [M + H]⁺ |
| 529 | 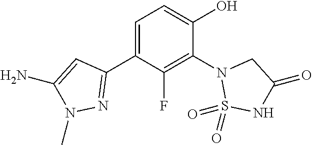 [SMILES: Cn1nc(-c2ccc(O)c(N3CC(=O)NS3(=O)=O)c2F)cc1N] | 5-(3-(5-amino-1-methyl-1H-pyrazol-3-yl)-2-fluoro- 6-hydroxyphenyl)-1,2,5-thiadiazolidin-3-one 1,1- dioxide | 340.0 |
| 530 | 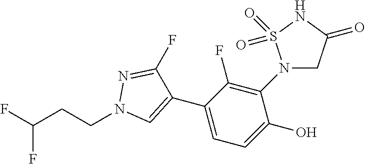 [SMILES: O=C1CN(c2c(O)ccc(-c3cn(CCC(F)F)nc3F)c2F)S(=O)(=O)N1] | 5-(3-(1-(3,3-difluoropropyl)-3-fluoro-1H-pyrazol-4- yl)-2-fluoro-6-hydroxyphenyl)-1,2,5-thiadiazolidin- 3-one 1,1-dioxide | 407.0 |
| 531 | 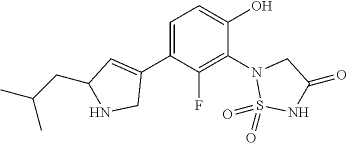 [SMILES: CC(C)CC1C=C(c2ccc(O)c(N3CC(=O)NS3(=O)=O)c2F)CN1] | 5-(2-fluoro-6-hydroxy-3-(5-isobutyl-2,5-dihydro-1H- pyrrol-3-yl)phenyl)-1,2,5-thiadiazolidin-3-one 1,1- dioxide | 368.1 |
| 532 | 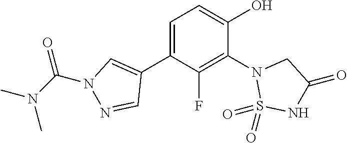 [SMILES: CN(C)C(=O)n1cc(-c2ccc(O)c(N3CC(=O)NS3(=O)=O)c2F)cn1] | 4-(3-(1,1-dioxido-4-oxo-1,2,5-thiadiazolidin-2-yl)-2- fluoro-4-hydroxyphenyl)-N,N-dimethyl-1H- pyrazole-1-carboxamide | 382.2 |
| 533 | 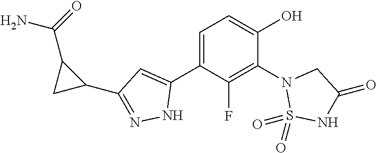 [SMILES: NC(=O)C1CC1c1cc(-c2ccc(O)c(N3CC(=O)NS3(=O)=O)c2F)[nH]n1] | 2-(5-(3-(1,1-dioxido-4-oxo-1,2,5-thiadiazolidin-2- yl)-2-fluoro-4-hydroxyphenyl)-1H-pyrazol-3- yl)cyclopropane-1-carboxamide | 394.0 |
| 534 | 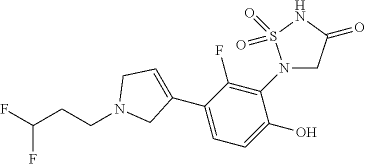 [SMILES: O=C1CN(c2c(O)ccc(C3=CCN(CCC(F)F)C3)c2F)S(=O)(=O)N1] | 5-(3-(1-(3,3-difluoropropyl)-2,5-dihydro-1H-pyrrol- 3-yl)-2-fluoro-6-hydroxyphenyl)-1,2,5- thiadiazolidin-3-one 1,1-dioxide | 390.1 |
| 535 | 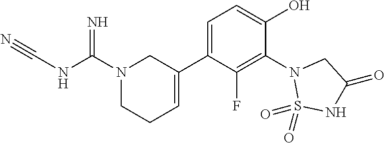 [SMILES: N#CNC(=N)N1CCC=C(c2ccc(O)c(N3CC(=O)NS3(=O)=O)c2F)C1] | N-cyano-5-(3-(1,1-dioxido-4-oxo-1,2,5- thiadiazolidin-2-yl)-2-fluoro-4-hydroxyphenyl)-3,6- dihydropyridine-1(2H)-carboximidamide | 393.1 |
| 536^{†} | 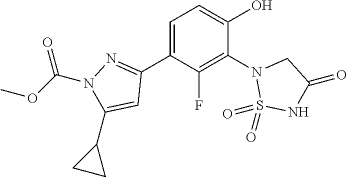 [SMILES: COC(=O)n1nc(-c2ccc(O)c(N3CC(=O)NS3(=O)=O)c2F)cc1C1CC1] | methyl 5-cyclopropyl-3-(3-(1,1-dioxido-4-oxo-1,2,5- thiadiazolidin-2-yl)-2-fluoro-4-hydroxyphenyl)-1H- pyrazole-1-carboxylate | 409.1 |
| 537 | 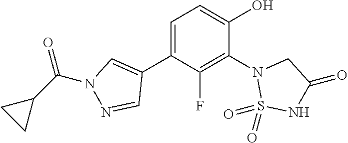 [SMILES: O=C1CN(c2c(O)ccc(-c3cnn(C(=O)C4CC4)c3)c2F)S(=O)(=O)N1] | 5-(3-(1-(cyclopropanecarbonyl)-1H-pyrazol-4-yl)-2- fluoro-6-hydroxyphenyl)-1,2,5-thiadiazolidin-3-one 1,1-dioxide | 379.0 |
| 538^{†} | 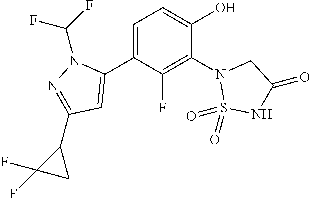 [SMILES: O=C1CN(c2c(O)ccc(-c3cc(C4CC4(F)F)nn3C(F)F)c2F)S(=O)(=O)N1] | 5-(3-(3-(2,2-difluorocyclopropyl)-1- (difluoromethyl)-1H-pyrazol-5-yl)-2-fluoro-6- hydroxyphenyl)-1,2,5-thiadiazolidin-3-one 1,1- dioxide | 439.1 [M + H]⁺ |
| 539 | 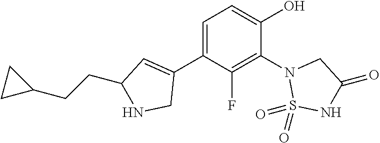 [SMILES: O=C1CN(c2c(O)ccc(C3=CC(CCC4CC4)NC3)c2F)S(=O)(=O)N1] | 5-(3-(5-(2-cyclopropylethyl)-2,5-dihydro-1H-pyrrol- 3-yl)-2-fluoro-6-hydroxyphenyl)-1,2,5- thiadiazolidin-3-one 1,1-dioxide | 380.1 |
| 540 | 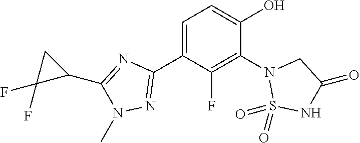 [SMILES: Cn1nc(-c2ccc(O)c(N3CC(=O)NS3(=O)=O)c2F)nc1C1CC1(F)F] | 5-(3-(5-(2,2-difluorocyclopropyl)-1-methyl-1H- 1,2,4-triazol-3-yl)-2-fluoro-6-hydroxyphenyl)-1,2,5- thiadiazolidin-3-one 1,1-dioxide | 402.0 |
| 541 | 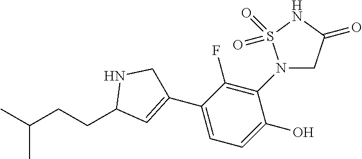 [SMILES: CC(C)CCC1C=C(c2ccc(O)c(N3CC(=O)NS3(=O)=O)c2F)CN1] | 5-(2-fluoro-6-hydroxy-3-(5-isopentyl-2,5-dihydro- 1H-pyrrol-3-yl)phenyl)-1,2,5-thiadiazolidin-3-one 1,1-dioxide | 382.1 |
| 542 | 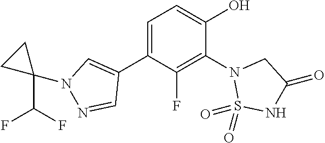 [SMILES: O=C1CN(c2c(O)ccc(-c3cnn(C4(C(F)F)CC4)c3)c2F)S(=O)(=O)N1] | 5-(3-(1-(1-(difluoromethyl)cyclopropyl)-1H-pyrazol- 4-yl)-2-fluoro-6-hydroxyphenyl)-1,2,5- thiadiazolidin-3-one 1,1-dioxide | 401.0 |
| 543 | 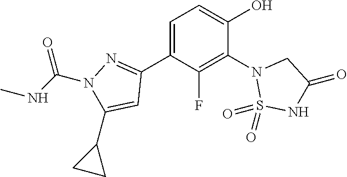 [SMILES: CNC(=O)n1nc(-c2ccc(O)c(N3CC(=O)NS3(=O)=O)c2F)cc1C1CC1] | 5-cyclopropyl-3-(3-(1,1-dioxido-4-oxo-1,2,5- thiadiazolidin-2-yl)-2-fluoro-4-hydroxyphenyl)-N- methyl-1H-pyrazole-1-carboxamide | 408.2 |
| 544 | 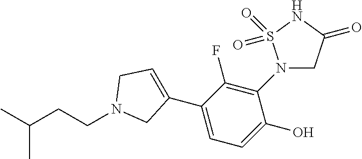 [SMILES: CC(C)CCN1CC=C(c2ccc(O)c(N3CC(=O)NS3(=O)=O)c2F)C1] | 5-(2-fluoro-6-hydroxy-3-(1-isopentyl-2,5-dihydro- 1H-pyrrol-3-yl)phenyl)-1,2,5-thiadiazolidin-3-one 1,1-dioxide | 382.1 |
| 545^{†} | 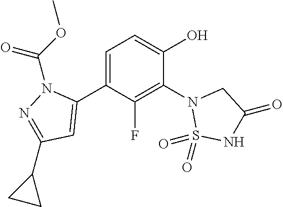 [SMILES: COC(=O)n1nc(C2CC2)cc1-c1ccc(O)c(N2CC(=O)NS2(=O)=O)c1F] | methyl 3-cyclopropyl-5-(3-(1,1-dioxido-4-oxo-1,2,5- thiadiazolidin-2-yl)-2-fluoro-4-hydroxyphenyl)-1H- pyrazole-1-carboxylate | 409.1 |
| 546 | 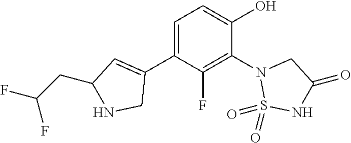 [SMILES: O=C1CN(c2c(O)ccc(C3=CC(CC(F)F)NC3)c2F)S(=O)(=O)N1] | 5-(3-(5-(2,2-difluoroethyl)-2,5-dihydro-1H-pyrrol-3- yl)-2-fluoro-6-hydroxyphenyl)-1,2,5-thiadiazolidin- 3-one 1,1-dioxide | 376.0 |
| 547 | 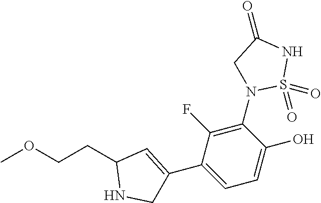 [SMILES: COCCC1C=C(c2ccc(O)c(N3CC(=O)NS3(=O)=O)c2F)CN1] | 5-(2-fluoro-6-hydroxy-3-(5-(2-methoxyethyl)-2,5- dihydro-1H-pyrrol-3-yl)phenyl)-1,2,5-thiadiazolidin- 3-one 1,1-dioxide | 370.0 |

^{†}Compound provided as a substantially pure single regioisomer, though the structure provided has been tentatively assigned.

In some instances, particularly for compounds denoted with t, two or more regioisomers may exist and the compound structures and corresponding chemical names provided in Table 2 have been tentatively assigned. In the preparation of certain compounds herein, such as compounds comprising a pyrazole, imidazole, or triazole, the starting material may exist in two or more tautomeric forms, thus the products may be isolated from the respective reaction mixtures as a single regioisomer or as a mixture of regioisomers that result from reaction with the two or more tautomeric forms of the starting material.

### Example 2: A Phosphatase Activity Assay

### Assessing Selectivity and Potency of a Small Molecule PTPN2 Inhibitor

The selectivity and potency of a small molecule PTPN2 inhibitor as provided herein against one or more protein tyrosine phosphatase (PTP) enzymes, and specifically against PTPN2, is assessed in various ways. The one or more PTP enzymes comprise Mycobacterium protein tyrosine phosphatase A (mPTPA), Mycobacterium protein tyrosine phosphatase B (mPTPB), PTPN1 (i.e., PTP1B), PTPN2 (i.e., TC-PTP), PTPN22 (i.e., LYP), SHP-1, SHP-2, FAP-1, Meg2, HePTP, Laforin, VHX, VHR, LMWPTP, Cdc14A, LAR, CD45, PTPRG, a fragment thereof, a variant thereof, and a combination thereof. Selectivity and potency of a small molecule PTPN2 inhibitor is evaluated using a PTP activity inhibition assay. The assay is performed using a buffer comprising 50 mM HEPES buffer pH 7.5, 150 mM NaCl, 0.5 mM EDTA, 1 mM DTT, 0.001% Tween-20.

The assay is performed using a phosphated substrate, e.g., 10 mM 6,8-Difluoro-4-Methylumbelliferyl Phosphate (DIFMUP) that is stored at −20° C. Alternatively or in addition to, the assay is performed using other phosphated substrate (e.g., fluorescein diphosphate). Each PTP enzyme is diluted in the assay buffer

The assay can be carried out at room temperature in multiplate format, e.g., using 384 well plates. A small molecule PTPN2 inhibitor dissolved in DMSO at one of 10 concentrations from a serial dilution or DMSO alone for control is added to each well. A mixture of the assay buffer comprising PTP enzyme (e.g., 0.025 ng/ul PTPN2) is added to each well and mixed for approximately 2 minutes. The reaction is initiated by adding DiFMUP diluted in the assay buffer to a final concentration of approximately 45 μM DiFMUP in PTPN2 assay. For each PTP enzyme, DiFMUP is added at the Km (Michaelis constant) of the enzyme that had been independently determined. The phosphatase activity of the PTP enzyme is assessed by monitoring appearance of a fluorescent product (6,8-difluoro-7-hydroxyl-4-coumarin (DiFMU) from DiFMUP) continuously for about 15 to 30 minutes, using the INFINITE M1000Pro plate reader (Tecan) with excitation at 360 nm and emission at 450 nm (cutoff filter at 435 nm) for DiFMU. Each assay is performed at least in duplicate. The rate (e.g., the initial rate) of DiFMU formation is plotted against the concentration of the small molecule PTPN2 inhibitor, and the data is fitted (e.g., using a 4-parameter equation) to determine the inflection point of the fit as the IC₅₀ of the small molecule PTPN2 inhibitor for a specific enzyme. Utilizing this assay, the ability of the compounds in Table 2 to inhibit PTPN2 and PTP1B has been assessed. Table 3 summarizes the IC50 values.

TABLE 3

| | ≤1 uM | >1 uM |
|---|---|---|
| PTPN2 | 101, 102, 103, 104, 106, 107, | 105, 116, 121, 134, |
| IC₅₀ | 108, 109, 110, | 136, 145, 151, 153, |
| | 111, 112, 113, 114, 115, 117, | 164, 168, 184, 192, |
| | 118, 119, 120, | 198, 218, 221, 246, |
| | 122, 123, 124, 125, 126, 127, | 250, 270, 276, 358, |
| | 128, 129, 130, | 413, 419, 460, 468, |
| | 131, 132, 133, 135, 137, 138, | 472, 475, 487, 512, |
| | 139, 140, 141, | 519 |
| | 142, 143, 144, 146, 147, 148, | |
| | 149, 150, 152, | |
| | 154, 155, 156, 157, 158, 159, | |
| | 160, 161, 162, | |
| | 163, 165, 166, 167, 169, 170, | |
| | 171, 172, 173, | |
| | 174, 175, 176, 177, 178, 179, | |
| | 180, 181, 182, | |
| | 183, 185, 186, 187, 189, 190, | |
| | 191, 193, 194, | |
| | 195, 196, 197, 199, 200, 201, | |
| | 202, 203, 204, | |
| | 205, 206, 207, 208, 209, 210, | |
| | 211, 212, 213, | |
| | 214, 215, 216, 217, 219, 220, | |
| | 222, 223, 224, | |
| | 225, 226, 227, 228, 229, 230, | |
| | 231, 232, 233, | |
| | 234, 235, 236, 237, 238, 239, | |
| | 240, 241, 242, | |
| | 243, 244, 245, 247, 248, 249, | |
| | 251, 252, 253, | |
| | 254, 255, 256, 257, 258, 259, | |
| | 260, 261, 262, | |
| | 263, 264, 265, 266, 267, 268, | |
| | 269, 271, 272, | |
| | 273, 274, 275, 277, 278, 279, | |
| | 280, 281, 282, | |
| | 283, 284, 285, 286, 287, 288, | |
| | 289, 290, 291, | |
| | 292, 293, 294, 295, 296, 297, | |
| | 298, 299, 300, | |
| | 301, 302, 303, 304, 305, 306, | |
| | 307, 308, 309, | |
| | 310, 311, 312, 313, 314, 315, | |
| | 316, 317, 318, | |
| | 319, 320, 321, 322, 323, 324, | |
| | 325, 326, 327, | |
| | 328, 329, 330, 331, 332, 333, | |
| | 334, 335, 336, | |
| | 337, 338, 339, 340, 341, 342, | |
| | 343, 344, 345, | |
| | 346, 347, 348, 349, 350, 351, | |
| | 352, 353, 354, | |
| | 355, 356, 357, 359, 360, 361, | |
| | 362, 363, 364, | |
| | 365, 366, 367, 368, 369, 370, | |
| | 371, 372, 373, | |
| | 374, 375, 376, 377, 378, 379, | |
| | 380, 381, 382, | |
| | 383, 384, 385, 386, 387, 388, | |
| | 389, 390, 391, | |
| | 392, 393, 394, 395, 396, 397, | |
| | 398, 399, 400, | |
| | 401, 402, 403, 404, 405, 406, | |
| | 407, 408, 409, | |
| | 410, 411, 412, 414, 415, 416, | |
| | 417, 418, 420, | |
| | 421, 422, 423, 424, 425, 426, | |
| | 427, 428, 429, | |
| | 430, 431, 432, 433, 434, 435, | |
| | 436, 437, 438, | |
| | 439, 440, 441, 442, 443, 444, | |
| | 445, 446, 447, | |
| | 448, 449, 450, 451, 452, 453, | |
| | 454, 455, 456, | |
| | 457, 458, 459, 461, 462, 463, | |
| | 464, 465, 466, | |
| | 467, 469, 470, 471, 473, 474, | |
| | 476, 477, 478, | |
| | 479, 480, 481, 482, 483, 484, | |
| | 486, 488, 489, | |
| | 490, 491, 492, 493, 494, 495, | |
| | 496, 497, 498, | |
| | 499, 500, 501, 502, 503, 504, | |
| | 505, 506, 507, | |
| | 508, 509, 510, 511, 513, 514, | |
| | 515, 516, 517, | |
| | 518, 520, 521, 522, 523, 524, | |
| | 525, 526, 527, | |
| | 528, 529, 530, 531, 532, 533, | |
| | 534, 535, 536, | |
| | 537, 538, 539, 540, 541, 542, | |
| | 543, 544, 545 | |
| PTP1B | 101, 102, 103, 104, 106, 107, | 105, 116, 121, 134, |
| IC₅₀ | 108, 109, 110, | 145, 164, 168, 184, |
| | 111, 112, 113, 114, 115, 117, | 218, 221, 246, 270, |
| | 118, 119, 120, | 358, 413, 419, 512, |
| | 122, 123, 124, 125, 126, 127, | 519 |
| | 128, 129, 130, | |
| | 131, 132, 133, 135, 136, 137, | |
| | 138, 139, 140, | |
| | 141, 142, 143, 144, 146, 147, | |
| | 148, 149, 150, | |
| | 151, 152, 153, 154, 155, 156, | |
| | 157, 158, 159, | |
| | 160, 161, 162, 163, 165, 166, | |
| | 167, 169, 170, | |
| | 171, 172, 173, 174, 175, 176, | |
| | 177, 178, 179, | |
| | 180, 181, 182, 183, 185, 186, | |
| | 187, 189, 190, | |
| | 191, 192, 193, 194, 195, 196, | |
| | 197, 198, 199, | |
| | 200, 201, 202, 203, 204, 205, | |
| | 206, 207, 208, | |
| | 209, 210, 211, 212, 213, 214, | |
| | 215, 216, 217, | |
| | 219, 220, 222, 223, 224, 225, | |
| | 226, 227, 228, | |
| | 229, 230, 231, 232, 233, 234, | |
| | 235, 236, 237, | |
| | 238, 239, 240, 241, 242, 243, | |
| | 244, 245, 247, | |
| | 248, 249, 250, 251, 252, 253, | |
| | 254, 255, 256, | |
| | 257, 258, 259, 260, 261, 262, | |
| | 263, 264, 265, | |
| | 266, 267, 268, 269, 271, 272, | |
| | 273, 274, 275, | |
| | 276, 277, 278, 279, 280, 281, | |
| | 282, 283, 284, | |
| | 285, 286, 287, 288, 289, 290, | |
| | 291, 292, 293, | |
| | 294, 295, 296, 297, 298, 299, | |
| | 300, 301, 302, | |
| | 303, 304, 305, 306, 307, 308, | |
| | 309, 310, 311, | |
| | 312, 313, 314, 315, 316, 317, | |
| | 318, 319, 320, | |
| | 321, 322, 323, 324, 325, 326, | |
| | 327, 328, 329, | |
| | 330, 331, 332, 333, 334, 335, | |
| | 336, 337, 338, | |
| | 339, 340, 341, 342, 343, 344, | |
| | 345, 346, 347, | |
| | 348, 349, 350, 351, 352, 353, | |
| | 354, 355, 356, | |
| | 357, 359, 360, 361, 362, 363, | |
| | 364, 365, 366, | |
| | 367, 368, 369, 370, 371, 372, | |
| | 373, 374, 375, | |
| | 376, 377, 378, 379, 380, 381, | |
| | 382, 383, 384, | |
| | 385, 386, 387, 388, 389, 390, | |
| | 391, 392, 393, | |
| | 394, 395, 396, 397, 398, 399, | |
| | 400, 401, 402, | |
| | 403, 404, 405, 406, 407, 408, | |
| | 409, 410, 411, 412, 413, 414, | |
| | 415, 416, 417, | |
| | 418, 418, 420, 421, 422, | |
| | 423, 424, 425, 426, 427, | |
| | 428, 429, 430, 431, | |
| | 432, 433, 434, 435, 436, | |
| | 437, 438, 439, 440, | |
| | 441, 442, 443, 444, 445, | |
| | 446, 447, 448, 449, | |
| | 450, 451, 452, 453, 454, | |
| | 455, 456, 457, 458, | |
| | 459, 460, 461, 462, 463, | |
| | 464, 465, 466, 467, | |
| | 468, 469, 470, 471, 472, | |
| | 473, 474, 475, 476, | |
| | 477, 478, 479, 480, 481, | |
| | 482, 483, 484, 486, | |
| | 487, 488, 489, 490, 491, | |
| | 492, 493, 494, 495, | |
| | 496, 497, 498, 499, 500, | |
| | 501, 502, 503, 504, | |
| | 505, 506, 507, 508, 509, | |
| | 510, 511, 513, 514, | |
| | 515, 516, 517, 518, 520, | |
| | 521, 522, 523, 524, | |
| | 525, 526, 527, 528, 529, | |
| | 530, 531, 532, 533, | |
| | 534, 535, 536, 537, 538, | |
| | 539, 540, 541, 542, 543, | |
| | 544, 545 | |

### Example 3: Cytokine Release Assay

Effector cells (e.g., non-modified T cells, or TFP- or CAR-expressing T cells) are activated with CD3/CD28 beads (CD3/CD28 CTS Dynabeads) in the presence of a small molecule PTPN2 inhibitor for 12-24 hours (optionally longer). The activated effector cells are induced to release cytokine by co-culturing with target cells at a desired Effector:Target cell ratio, e.g., 10:1, 5:1, or 1:1. Co-culture supernatant is harvested after approximately 20 hrs. These supernatants are then used to measure the released cytokines such as IL-2 and IFN-g, using Meso Scale Discovery, Proinflammatory Panel 1 catalog #N05049A-1 system according to the manufacturer's protocol. The target cells can be irradiated (e.g. at 10,000) prior to co-culturing with the effector cells. This assay is to demonstrate that inhibiting PTPN2 by a small molecule PTPN2 inhibitor causes an increase in cytokine release (e.g., IL-2 or IFN-g) by T cells in response to the antigen to which the TFP or CAR binds.

### Example 4: Cell Proliferation Assay

Effector cells (e.g., non-modified T cells, or TFP- or CAR-expressing T cells) are activated with CD3/CD28 beads (CD3/CD28 CTS Dynabeads) in the presence of a small molecule PTPN2 inhibitor for 12-24 hours (optionally longer). The activated effector cells are induced to proliferate by co-culturing with target cells that comprise the target tumor antigen to which the TFP or CAR binds. Typically the target cells are irradiated, washed and counted. Co-culturing is performed at a desired Effector: Target cell ratio, e.g., 10:1, 5:1, or 1:1. Proliferation of the effector cells are evaluated, typically after bead expansion for about 10 days. The number of cells per mL and the viability of cells are measured by Cellometer. This example is to demonstrate that PTPN2 inhibition by a small molecule PTPN2 inhibitor yields an increase in effector cell number and viability relative to effector cells not treated with a small molecule PTPN2 inhibitor.

### Example 5: Cytotoxicity Assay

Effector cells (e.g., non-modified T cells, or TFP- or CAR-expressing T cells) are activated with CD3/CD28 beads (CD3/CD28 CTS Dynabeads) in the presence of a PTPN2 inhibitor (e.g., compound described herein) for 12-24 hours (optionally longer). Target cells (e.g., cancer or tumor cells) that comprise the target tumor antigen to which the TFP or CAR binds are incubated with Calcein-AM in the dark, washed, and counted. The activated effector cells are co-cultured with the target cells. Co-culturing is performed at a desired Effector: Target cell ratio, e.g., 10:1, 5:1, or 1:1. At the end of the co-culture period (e.g., 5 hours), the number of target cells and the viability of the target cells are assessed by measuring Calcein fluorescence from the collected cells. This example is to demonstrate that PTPN2 inhibition by a PTPN2 inhibitor (e.g., compound described herein) yields an increase in cytotoxicity of effector cells against target cells relative to effector cells not treated with a PTPN2 inhibitor (e.g., compound described herein).

### Example 6: CAR-T Killing Assay

The ability of PTPN2 inhibitors to potentiate tumor cell killing using CAR-Ts that express a tumor antigen (e.g., HER2) is demonstrated as follows. HER-2 specific CAR-T cells (CAR-Ts) are generated by transducing primary human CD3+ T cells (Discovery Life Sciences) with a lentivirus expressing a chimeric antigen receptor specific to human HER2, as well as GFP (Creative Bio). Prior to transduction, T cells are stimulated overnight with anti-CD3 and anti-CD28 antibodies coated onto magnetic beads (Invitrogen) at a 1:1 bead-to-cell ratio. Four days after transduction, the beads are removed and the following day CAR-Ts were sorted based on GFP expression and expanded in hIL7 (10 ng/mL) (Peprotech) and hIL15 (5 ng/mL) (Peprotech) for an additional 8 days. Thereupon, CAR-Ts are co-cultured with a Nuclight Red (Essen Biosciences)-labeled HER-2 positive tumor line (OVCAR-3) or a HER-2 negative line (HEK293T) for 18 h at a 1:1 effector:target cell ratio.

CAR-Ts are pretreated with 0.1% DMSO (vehicle control) or a PTPN2 inhibitor for 1-2.5 hours prior to co-culture with cell lines at indicated concentrations. PTPN2 inhibitor is washed from CAR-Ts prior to inclusion in the tumor killing assay. Tumor killing is assessed by comparing DMSO-treated CAR-Ts to PTPN2 inhibitor-treated CAR-Ts using either flow cytometry or Incucyte imaged-based assessment of tumor cell viability, at various effector:target ratios. Percent killing is assessed by calculating the amount of viable cells at a given time point as compared to untreated tumor cells, tumor cells treated with PTPN2 inhibitor, and tumor cells cultured with DMSO treated CAR-T cells as a control. The results are expected to demonstrate that: (a) PTPN2 treated CAR-T cells exhibit a higher tumor cell killing activity as compared to control cells treated with DMSO; and (b) even a transient treatment of PTPN2 (e.g., for an hour followed by washing) is sufficient to potentiate the ability of CAR-T cells to kill tumor cells.

### Example 7: CAR-T Adoptive Cell Transfer Xenograft Tumor Assay

For in vivo studies with CAR-Ts, nude mice are implanted with OVCAR xenografts. After reaching a suitable size of 50-100 mm³, approximately 10⁶ CAR-Ts transiently treated (e.g., for 1 hr and then washing away) with or without PTPN2 inhibitor are transferred i.v. into tumor bearing mice. Tumor volume and CAR-T cell count are measured at multiple time points and compared to control groups treated with DMSO (e.g., for 1 hr and then washing away). It is expected that mice administered with CAR-Ts that are treated with PTPN2 inhibitor exhibit lower tumor volume, higher frequency of CAR-Ts in the blood, and/or greater infiltration and/or activation of CAR-Ts into tumors, smaller tumor volume and/or higher CAR-T cell counts.

### Example 8: Mouse Adoptive Cell Transfer Syngeneic Tumor Assay

Thy1.1 congenic C57BL/6 mice are implanted with approximately 5×10⁵ of OVA expressing syngeneic tumor cells (B16-OVA, EL4 OVA, or YUMM1.1) formulated with 50% Matrigel (50% PBS). Prior to the implantation, the B16-OVA, EL4 OVA, or YUMM1.1 tumor cell lines are transduced with a lentivirus encoding an OVA-GFP fusion protein. After sorting for GFP expression, the B16-OVA, EL4 OVA, or YUMM1.1 cells are shown to grow in untreated C57BL/6 mice. After growing to a volume of ˜-50-100 mm³, tumor bearing mice would receive i.v. transfer of approximately 1×10⁶ OT-1 transgenic T cells that will undergo the following treatments. First, primary OT-1 splenocytes are treated with 10 nm of SIINFEKL peptide or anti-CD3/anti-CD28 coated beads. After 2 days, the cells are washed and transferred into culture medium with IL-2, IL-7 and IL-15 (all at 5 ng/ml) for another 3 days. In other experiments, naïve OT-1 CD8 T cells are isolated for transfer. Prior to the transfer, the OT-1 cells are treated with DMSO (vehicle control) or a PTPN2 inhibitor for 1 hour and washed in PBS two times prior to injection.

The test groups are separated as follows: Tumor alone, Tumor+DMSO treated OT-1s, Tumor+PTPN2 inhibitor treated OT-Is. Each group may include 8 mice for the 2 time points tested. To assess in vivo efficacy, tumor volume is measured 3×/week using calipers at various time points post OT-1 injection. Furthermore, at day 7, the first group of mice are sacrificed to compare immune activation and infiltration in both secondary lymphoid tissue and in tumors, staining for markers including but not limited to CD4, CD8, CD25, CD69, CD44, CD62L, TCF1, TOX, TIM3, PD1. The abundance and activation state of immune cells are quantified using flow cytometry. The results are expected to demonstrate that a transient treatment with PTPN2 is sufficient to potentiate anti-tumor killing as evidenced by (a) a decrease in tumor volume, and/or (b) an increase in the abundance of activated T cells in spleen, lymph nodes and/or in tumor.

### Example 9: Mobility Shift Assay Used to Determine Potency of PTPN2 Inhibitors

Compound activity can be determined using PTPN2 in an in vitro enzymatic reaction. The enzymatic assay used to determine activity can be a mobility shift assay using a LabChip EZ Reader by Caliper Life Sciences. The enzymatic reaction is carried out in assay buffer (50 mM HEPES pH 7.5, 1 mM EGTA, 10 mM EDTA, 0.01% Tween® 20, and 2 mM DTT). The compounds are dispensed on a white 384 well ProxiPlate™ (PerkinElmer Catalog #6008289) plate using the Labcyte Echo at varying concentrations (12 point, 1:3 dilution). The enzyme (at 0.5 nM) is incubated with compound for 10 minutes at room temperature. Then the substrate (phosphorylated insulin receptor probe sequence is added at 2 μM to the plates and incubated for another 10 minutes at room temperature. Finally, a quench solution (water and 4-bromo-3-(2-oxo-2-propoxyethoxy)-5-(3-{[1-(phenylmethanesulfonyl)piperidin-4-yl]amino}phenyl)thiophene-2-carboxylic acid) is added to the plates, which are then run on the EZ Reader (excitation 488 nm, emission 530 nm) to measure % conversion (the amount of phosphorylated substrate which is de-phosphorylated by PTPN2). Each plate has a 100% control (inhibitor: 4-bromo-3-(2-oxo-2-propoxyethoxy)-5-(3-{[1-(phenylmethanesulfonyl)piperidin-4-yl]amino}phenyl)thiophene-2-carboxylic acid) and 0% control (DMSO), which are used to calculate % inhibition. The % inhibition is then used to calculate the IC50 values.

### Example 10: Mobility Shift Assay (MSA) to Determine Potency of PTPN1 (Also Known as PTP1B) Inhibitors

Compound activity can be determined using full-length PTPN1 protein in an in vitro enzymatic reaction. The enzymatic assay to determine activity can be a mobility shift assay using a LabChip EZ Reader by Caliper Life Sciences. The enzymatic reaction is carried out in assay buffer (50 mM HEPES pH 7.5, 1 mM EGTA, 10 mM EDTA, 0.01% Tween® 20, and 2 mM DTT). The compounds are dispensed on a white 384 well ProxiPlate™ (PerkinElmer Cat #6008289) plate using a Labcyte Echo® liquid handler at varying concentrations (12 point, 1:3 dilution). The enzyme (at 0.5 nM) is incubated with compound for 10 minutes at room temperature. Then the substrate (phosphorylated insulin 30 receptor probe sequence is added at 2 μM to the plates and incubated for another 10 minutes at room temperature. Finally, a quench solution (water and 4-bromo-3-(2-oxo-2-propoxyethoxy)-5-(3-{[1-(phenyhnethanesulfonyl)piperidin-4-yl] amino}phenyl)thiophene-2-carboxylic acid) is added to the plates, which are then run on the EZ Reader (excitation 488 nm, emission 530 nm) to measure % conversion (the amount of phosphorylated substrate which is de-phosphorylated by PTPN1). Each plate has a 100% control (inhibitor: 4-bromo-3-(2-oxo-2-propoxyethoxy)-5-(3-{[l-(phenyhnethanesulfonyl)piperidin-4-yl]amino}phenyl)thiophene-2-carboxylic acid) and 0% control (DMSO), which are used to calculate % inhibition. The % inhibition is then used to calculate the IC50 values.

### Example 11: B16F10 IFNγ-Induced MHC Upregulation and Cellular Growth Inhibition

B16F10 mouse melanoma cells (ATCC Cat #CRL-6475, Manassas, VA) are seeded at a density of 500 cells per well in a 384-well clear bottom plate in 25 μL total volume of DMEM+10% FBS. Cells are allowed to adhere overnight at 37° C.+5% CO₂. On the following day, 12.5 μL of mouse IFNγ is added to half of the plate at a concentration of 2 ng/mL for a final assay concentration of 0.5 ng/mL of IFNγ. Media only (12.5 μL of DMEM+10% FBS) is added to the remainder of the plate. Compounds resuspended in DMSO at 100 mM are diluted in DMSO ranging from 100 mM to 0.001 mM and DMSO only controls are included. The compound/DMSO dilutions are further diluted 1:250 in DMEM+10% FBS, and 12.5 μL of these dilutions are added in triplicates to cells with or without IFNγ. Final compound concentrations range from 100 μM to 0.001 μM with a final DMSO concentration of 0.1%. The outer 2-well perimeter of the plate is not used. The plate is loaded into an IncuCyte® S3 Live Cell Analysis System (Essen Bioscience-Sartorius, Ann Arbor, MI) maintained in a 37° C.+5% CO₂ incubator, allowed to equilibrate for 2 hours, and imaged every 6 hours for 5 days. Confluence over time for compound dilutions in the presence and absence of IFNγ is measured. Growth inhibition values are obtained when the “DMSO/no IFNγ” control reaches confluence >95%. At these time points, the percent growth inhibition of each compound at the indicated concentration is calculated relative to the “DMSO/with IFNγ” control. At these same time points, MHC upregulation is assessed by flow cytometry. Compounds inhibiting PTPN2 are expected to show an increase in MHC levels in a dose-dependent manner.

### Example 12: T Cell Activation and Function

Pan T cells are isolated from C57BL6 splenocytes. Isolated T cells (50,000 cells/well in a 96 well flat-bottom plate) are cultured in RPMI 1640 supplemented with 10% FBS, 50 nM 2-mercatoethanol, 100 U/mL penicillin, and 100 μg/mL streptomycin, and incubated with the indicated concentration of compound or DMSO in duplicates. After 1 hour, mouse T cell activator CD3/CD28 Dynabeads are added at a 1:5 beads to cells ratio to stimulate the T cells for 2 or 3 days as described below. Alternatively, antibodies to CD3 and CD28 are plate-bound for stimulation. T cells with or without compound are incubated in the absence of T cell activator beads (media only) as control. After 2 days of stimulation, activation status of T cells is assessed by flow cytometry. T cells are first subjected to Zombie Violet™ Fixable Viability dye for dead cell exclusion, washed and then stained with BUV805 labeled anti-CD8, APC-R700 labeled anti-CD25 and PE labeled anti-CD69 antibodies. After staining, cells are fixed with 2% paraformaldehyde and acquired on a BD LSRFortessa™ X-20 flow cytometer using BD FACSDiva™ software and data is analyzed. Dead cells are excluded and frequencies of activated CD8 T cells is reported as the frequency of CD25+ or CD69+ cells within the CD8+ population. The expression level of CD25 and CD69 indicates the activation status of cells on a per cell basis and is evaluated by the mean fluorescence intensities (MFI) of CD25 and CD69. After 3 days of stimulation, supernatants are collected and IFNγ and TNFα in supernatants are assessed using an MSD V-plex assay (Meso Scale Discovery, Rockville, MD).

### Example 13: In Vivo Efficacy of Compounds in MC38 and B16F10 Tumor Models

### Tumor Cell Inoculation and Treatments.

Cells are grown to passage 3 in vitro. A total of 1×10⁵ viable MC-38 or B16F10 cells are inoculated subcutaneously into the right flank of female C57B11/6 mice (7-12 weeks old) on Day 0. The injection volume is 0.1 mL and is composed of a 1:1 mixture of S-MEM and Matrigel® (Corning, NY, USA). Tumors are size matched on Day 14 and the mice have a mean body weight of ˜21 g. The mean tumor volume (TV) at size match is approximately 116±8 mm³. Following size match, treatments are initiated on the same day. Dosing of mice is conducted on a daily or twice daily schedule.

Tumor volume is calculated three times weekly. Measurements of the length (L) and width (W) of the tumor are taken via electronic caliper and the volume is calculated according to the following equation: V=L×W²/2 using Study Director Version 3.1.399.22 (Studylog Systems, Inc, CA, USA). Mice are euthanized when tumor volume is ≤3000 mm³ or skin ulcerations occurred. Tumor growth inhibition (TGI) is calculated as TGI=1-(Mean TVTimepoint (Treatment)/Mean TVTimepoint (Vehicle)) for each timepoint that tumor volumes are measured. Reported TGIMax is the largest TGI value for any timepoint that tumors volumes are collected for that treatment group. Where desired, at approximately day 7, a first group of mice are sacrificed to compare immune activation and infiltration in both secondary lymphoid tissue and in tumors, staining for markers including but not limited to CD4, CD8, CD25, CD69, CD44, CD62L, TCF1, TOX, TIM3, PD1. The abundance and activation state of immune cells are quantified using flow cytometry.

pSTAT5 Flow Cytometry Assay in Mouse Whole Blood

Whole blood is drawn into EDTA powder coated tubes by cardiac puncture from mice on day 7 of dosing with indicated compound. 100 μL of whole blood are stimulated with 100 ng/mL murine IL-2 for 20 minutes at 37° C., 5% CO₂. After stimulation, 1.8 mL of prewarmed BD Phosflow Lyse/Fix Buffer is added for 20 minutes at 37° C. Cells are washed twice in FACS buffer (Dulbecco's PBS with 0.2% BSA) and incubated for 30 minutes on ice in cold Perm Buffer III. Cells are washed with FACS buffer and resuspended in 50 μL of FACS buffer with antibodies and stained for 3 hours at room temperature with gentle shaking. The antibodies added are a combination of the following: anti-CD3- AF647, clone 145-2C11; anti-CD4-FITC, clone GK1.5; anti-pSTAT5 (pY694)-PE, clone 47; anti-CD45-BUV395, clone 30-F11. After staining, cells are washed twice with FACS buffer, and the samples are acquired on a BD LSRFortessa™ X20 flow cytometers (BD Biosciences, San Jose, CA) and analyzed with FLowJo V10 software (FlowJo, Ashland, OR). The mean fluorescence intensity (MFI) of pSTAT5 as a measure of the amount of phosphorylated STATS in the CD3+ T cell population is reported as fold-change of compound treated over vehicle treated animal groups.

Granzyme B staining of CD8 T cells Flow Cytometry Assay in Mouse Spleen

Mice are sacrificed on day 7 of dosing with compound and spleens are excised. Spleens are dissociated, red blood cells lysed, and single cell suspensions are prepared. Splenocytes are stained with Zombie UV™ Fixable Viability kit diluted in Dulbecco's PBS for 10 minutes at room temperature to exclude dead cells followed by staining for surface markers for 45 minutes on ice using the following flow cytometry antibodies diluted in autoMACS® Running Buffer (Miltenyi Biotec, Bergisch Gladbach, Germany): Brilliant Violet 510-labeled anti-CD45, Brilliant Ultraviolet 395-labeled anti-CD3, Brilliant Violet 786-labeled anti-CD4, APC/Cy7-labeled anti-CD8. Cells are washed twice with autoMACS® Running Buffer, permeabilized with Fixation/Permeabilization buffer (FoxP3/Transcription Factor Staining Buffer Set) and stained intracellularly with PE-labeled anti-Granzyme B antibody diluted in Permeabilization buffer (FoxP3/Transcription Factor Staining Buffer Set) for 1 hour on ice. After staining, cells are washed twice with autoMACS® Running Buffer, and the samples are acquired on a BD LSRFortessa™ X20 flow cytometers (BD Biosciences, San Jose, CA) and analyzed with FLowJo V10 software (FlowJo, Ashland, OR).

### Cytokine Measurement in Mouse Plasma

Whole blood is drawn into sodium heparin by cardiac puncture from mice on day 7 of dosing with compound and plasma is prepared by centrifugation. Cytokines in plasma are measured using the Th1/Th2 Cytokine & Chemokine 20-Plex Mouse ProcartaPlex™ Panel 1 (Invitrogen, Carlsbad, CA). IP10 levels are expressed as fold-changes over the vehicle control animal group.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the invention. It is intended that the following claims define the scope of the invention and that methods and structures within the scope of these claims and their equivalents be covered thereby.

The invention claimed is:

1-23. (canceled)

24. A compound of Formula (IIa), or a pharmaceutically acceptable salt or solvate thereof: 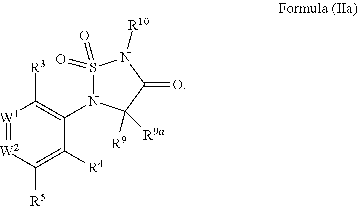
wherein:
W¹ is C(R¹), and W² is C(R^{2a}) or N;
R¹ is selected from C₅₋₁₂cycloalkyl, 5 to 12 membered heterocycloalkyl, C₆₋₁₂aryl, and 5 to 12 membered heteroaryl, wherein C₅₋₁₂cycloalkyl, 5 to 12 membered heterocycloalkyl, C₆₋₁₂aryl, and 5 to 12 membered heteroaryl are substituted with one or more substituents independently selected from R⁶ and R^{6′} wherein ring heteroatoms of the 5 to 12 membered heterocycloalkyl and 5 to 12 membered heteroaryl are independently selected from N, O, and S;
R^{2a} is independently selected from hydrogen, halogen, —CN, C₁₋₆alkyl, C₂₋₆alkenyl, C₂₋₆alkynyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, and C₂₋₆alkynyl are optionally substituted with one, two, or three R^{20b};
R³ is selected from halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —OR¹², —SR¹², —N(R¹²)(R¹³), —C(O)OR¹², OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynl, C₃₋₁₀cycloalkyl, C₂₋₉heterocycloalkyl, C₆₋₁₀aryl, and C₁₋₉heteroaryl are optionally substituted with one, two, or three R^{20c};
R⁴ is selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20d};
R⁵ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20e};
each R⁶ is independently selected from halogen, oxo, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶;
each R^{6′} is independently selected from —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶;
R⁹ and R^{9a} are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁰ⁱ or R⁹ and R^{9a} are combined to form a C₃₋₆cycloalkyl or a C₂₋₉heterocycloalkyl, wherein the C₃₋₆cycloalkyl and C₂₋₉heterocycloalkyl are optionally substituted with one, two, or three R²⁰ⁱ;
R¹⁰ is selected from hydrogen, halogen, —CN, C₁₋₆alkyl, C₂₋₆alkenyl, C₂₋₆alkynyl, C₃₋₁₀cycloalkyl, C₂₋₉ heterocycloalkyl, C₆₋₁₀aryl, C₁₋₉heteroaryl, —R¹⁶, —OR¹⁶, —N(R¹²)(R¹⁶), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl and C₁₋₉heteroaryl are optionally substituted with one, two, or three R^{20j};
each R¹² is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₁₀aryl, and C₁₋₉ heteroaryl are optionally substituted with one, two, or three R^{20k};
each R¹³ is independently selected from hydrogen, C₁₋₆alkyl, and C₁₋₆haloalkyl: or R¹² and R¹³, together with the nitrogen to which they are attached, form a C₂₋₉heterocycloalkyl ring optionally substituted with one, two, or three R^{20l};
each R¹⁴ is independently selected from hydrogen, C₁₋₆alkyl, and C₁₋₆haloalkyl;
each R¹⁵ is independently selected from C₁₋₆alkyl, C₂₋₆alkenyl, C₂₋₆alkynyl, C₃₋₁₀cycloalkyl, C₁₋₆ heteroalkyl, C₂₋₉heterocycloalkyl, C₁₀aryl, and C₁₋₉heteroaryl, wherein C₁₋₆alkyl, C₂₋₆alkenyl, C₂₋₆alkynyl, C₃₋₁₀cycloalkyl, C₁₋₆heteroalkyl, C₂₋₉heterocycloalkyl, C₆₋₁₀aryl, and C₁₋₉heteroaryl are optionally substituted with one, two, or three R^{20m};
each R¹⁶ is independently selected from —C₁₋₆alkylene-OP(O)(OR¹⁶a)(OR_{16b}) and —P(O)(OR¹⁶a)(OR^{16b}), wherein the C₁₋₆alkylene is optionally substituted with one, two, or three R²⁰ⁿ, or —C₁₋₆alkylene-OC(O)—R^{16c} wherein C₁₋₆alkylene is optionally substituted with one, two, or three R²⁰ⁿ;
each R^{16a} and R^{16b} is independently selected from hydrogen, C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl, wherein C₁₋₆alkyl, C₂₋₆alkenyl, C₂₋₆alkynyl, C₃₋₁₀cycloalkyl, —CH₂—C₃₋₁₀cycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three R^{20o};
R^{16c} is selected from halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, C₁₋₉ heteroaryl, —CH₂—C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉ heterocycloalkyl, —CH₂—C₂₋₉heterocycloalkyl, C₆₋₁₀aryl, —CH₂—C₆₋₁₀aryl, C₁₋₉heteroaryl, and —CH₂—C₁₋₉heteroaryl are optionally substituted with one, two, or three R^{20o};
each R^{20b}, R^{20c}, R^{20d}, R^{20e}, R²⁰ⁱ, R^{20j}, R^{20k}, R^{20l}, R^{20m}, R²⁰ⁿ, and R^{20o} is independently selected from oxo, halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉ heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl optionally substituted with one or more R²⁶, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl;
each R²² is independently selected from H, C₁₋₆alkyl, C₁₋₆haloalkyl, C₂₋₆alkenyl, C₂₋₆alkynyl, C₃₋₆cyclOalkyl, C₂₋₉heterocycloalkyl, C₆₋₁₀aryl, and C₁₋₉heteroaryl;
each R²³ is independently selected from H and C₁₋₆alkyl;
each R²⁴ is independently selected from H and C₁₋₆alkyl;
each R²⁵ is selected from C₁₋₆alkyl, C₂₋₆alkenyl, C₂₋₆alkynyl, C₃₋₁₀cycloalkyl, C₁₋₆heteroalkyl, C₂₋₉ heterocycloalkyl, C₆₋₁₀aryl, and C₁₋₉heteroaryl;
each R²⁶ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁷;
each R²⁷ is independently selected from oxo, halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20f}; and
each R^{20f} is independently selected from oxo, halogen, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²², —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉ heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵.

25-27. (canceled)

28. The compound of claim 24, or a pharmaceutically acceptable salt or solvate thereof, wherein R⁴ is —OH.

29. (canceled)

30. (canceled)

31. The compound of claim 24, or a pharmaceutically acceptable salt or solvate thereof, wherein R⁵ is hydrogen.

32. (canceled)

33. The compound of claim 24, or a pharmaceutically acceptable salt or solvate thereof wherein R³ is halogen.

34. The compound of claim 24, or a pharmaceutically acceptable salt or solvate thereof wherein R³ is F.

35. (canceled)

36. The compound of claim 24, or a pharmaceutically acceptable salt or solvate thereof wherein R¹ is 5 to 12 membered partially unsaturated heterocycloalkyl substituted with one or more substituents independently selected from R⁶ and R^{6′}.

37. (canceled)

38. The compound of claim 24, or a pharmaceutically acceptable salt or solvate thereof wherein R¹ is 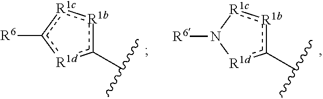
wherein R^{1c} is not N or N(R^{6c″}) when R^{1b} is C(R^{6b}) and R^{1d} is C(R^{6d}); 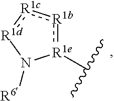
wherein R¹ is not a substituted pyrrolyl; 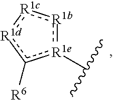
wherein R¹ is not a substituted imidazolyl wherein R^{1d} and R^{1e} are N; or
fluoro-substituted pyrazolyl optionally further substituted with one, two, or three substituents independently selected from R⁶ and R^{6′};
R^{1b} is O, S, N, N(R^{6b″}), C(R^{6b}), or C(R^{6b})(R^{6b′});
R^{1c} is O, S, N, N(R^{6c″}), C(R^{6c}), or C(R^{6c})(R^{6c″});
R^{1d} is O, S, N, N(R^{6d″}), C(R^{6d}), or C(R^{6d})(R^{6d′});
R^{1e} is N or C(R^{6e});
R^{6b}, R^{6b′}, R^{6c}, R^{6c′}, R^{6d}, R^{6d′}, and R^{6e} e are independently selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₁₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉ heterocycloalkyl, C₆₋₁₀aryl, and C₁₋₉heteroaryl are optionally substituted with one, two, or three R²⁶;
R^{6b″}, R^{6c″}, and R^{6d″} are independently selected from hydrogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, —OR¹², —C(O)OR¹², —OC(O)N(R¹²)(R¹³), —C(O)R¹⁵, —OC(O)R¹⁵, —C(O)N(R¹²)(R¹³), —C(O)C(O)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁶; and [symbol]
indicates a single or double bond such that all valences are satisfied.

39-43. (canceled)

44. The compound of claim 24, or a pharmaceutically acceptable salt or solvate thereof, wherein R¹ is 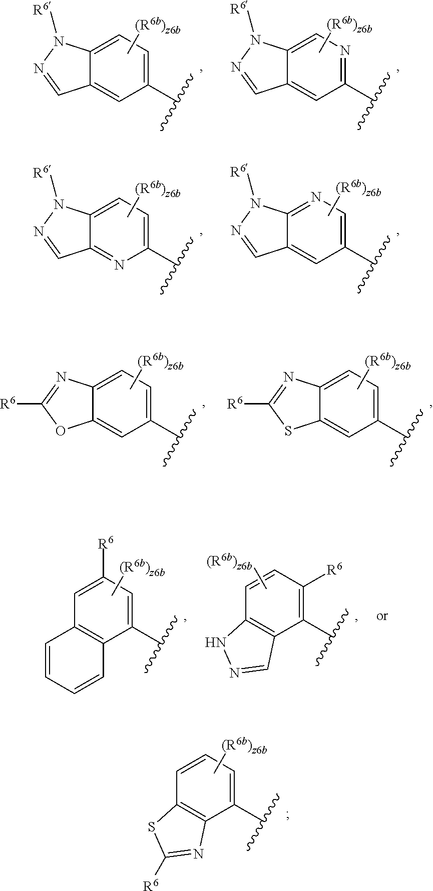
R^{6b} is independently selected from halogen, —CN, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹², —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl are optionally substituted with one, two, or three R²⁶; and
z6b is an integer from 0 to 4.

45. (canceled)

46. The compound of claim 34, or a pharmaceutically acceptable salt or solvate thereof, wherein R¹ is pyrazolyl substituted with one or more substituents independently selected from R⁶ and R^{6′}.

47-52. (canceled)

53. The compound of claim 24, or a pharmaceutically acceptable salt or solvate thereof, wherein R⁶ is independently selected from C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R²⁶.

54-57. (canceled)

58. The compound of claim 24, or a pharmaceutically acceptable salt or solvate thereof, wherein R⁶ is independently selected from C₁₋₆alkyl,

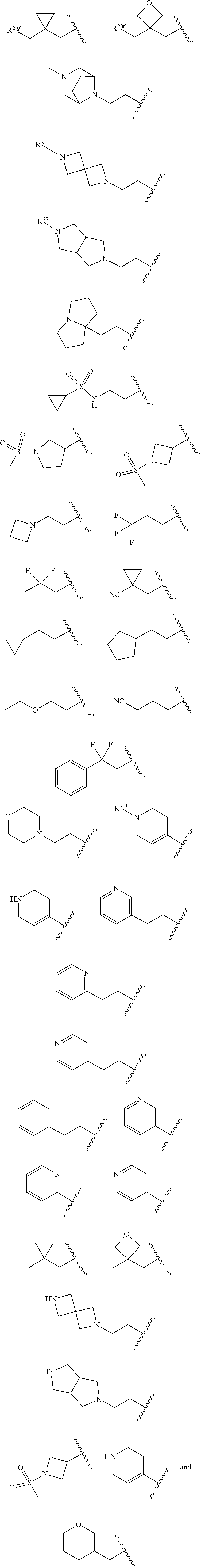

59. The compound of claim 24, or a pharmaceutically acceptable salt or solvate thereof, wherein R⁶ is independently selected from

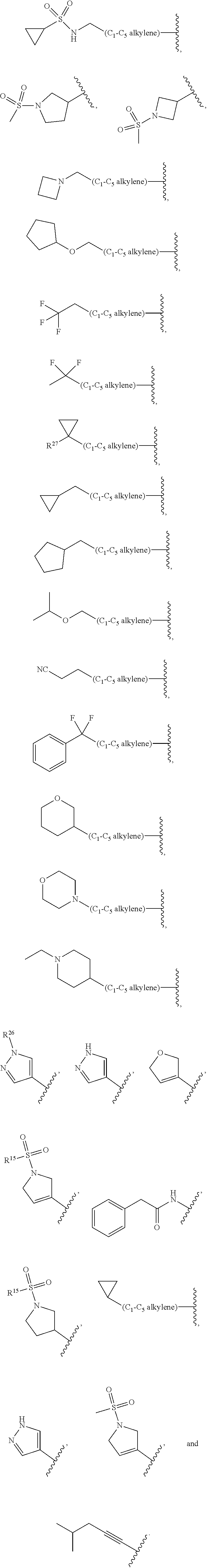

60. (canceled)

61. (canceled)

62. The compound of claim 24, or a pharmaceutically acceptable salt or solvate thereof, wherein R⁶ is

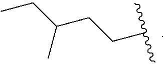 [SMILES: *CCC(C)CC]

63. The compound of claim 24, or a pharmaceutically acceptable salt or solvate thereof wherein W² is CH, R⁹ is hydrogen, R^{9a} is hydrogen, and R¹⁰ is hydrogen.

64. (canceled)

65. The compound of claim 24, or a pharmaceutically acceptable salt or solvate thereof, having the structure of Formula (IId-1):

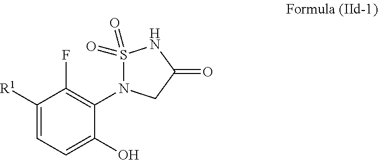

66. The compound of claim 24, or a pharmaceutically acceptable salt or solvate thereof, wherein R¹ is selected from pyrrolyl, pyrazolyl, imidazolyl, triazolyl, pyrrolinyl, pyridinyl, tetrahydropyridinyl, piperidinyl, and piperazinyl, each of which is substituted with one or more R⁶.

67. The compound of claim 24, or a pharmaceutically acceptable salt or solvate thereof, wherein R¹ is selected from

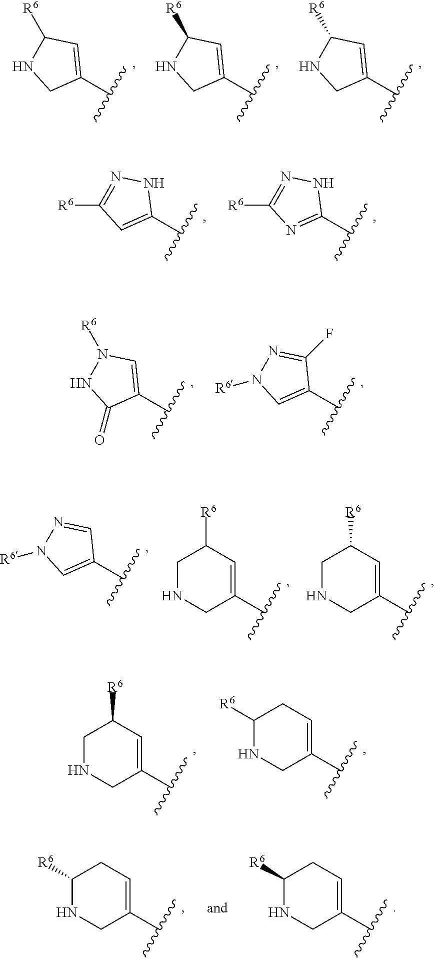

68. The compound of claim 24, wherein the compound is selected from: 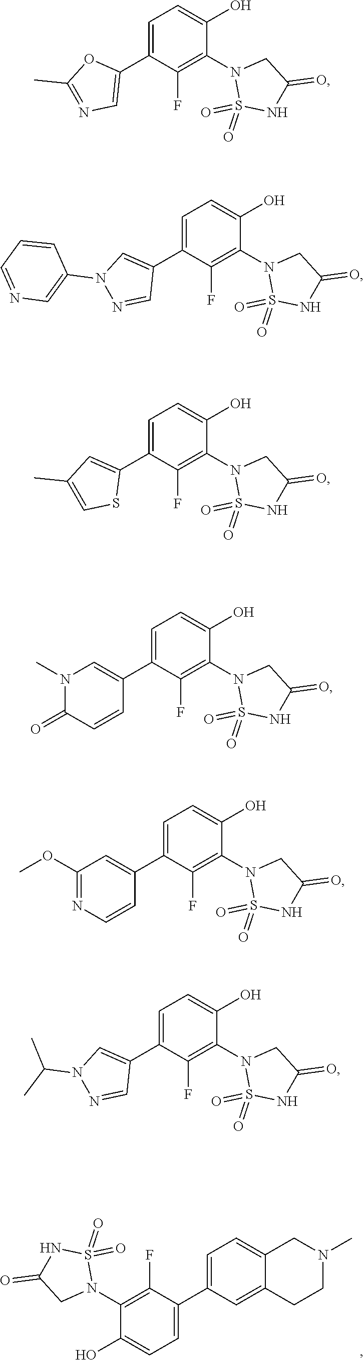 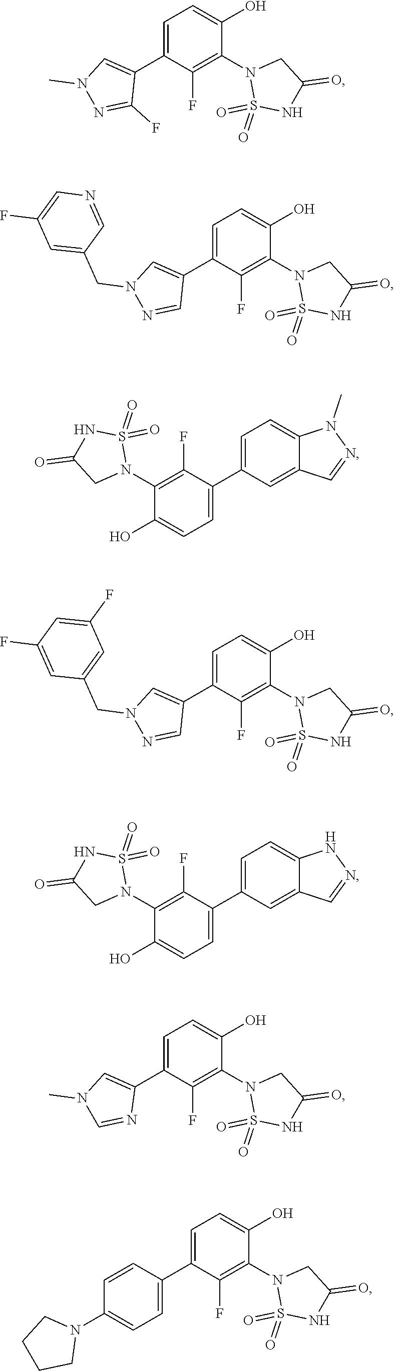 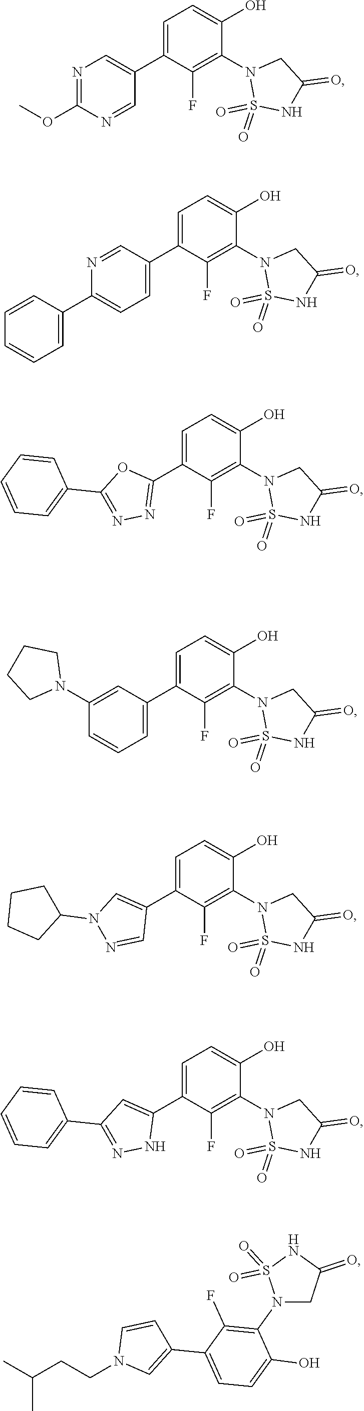 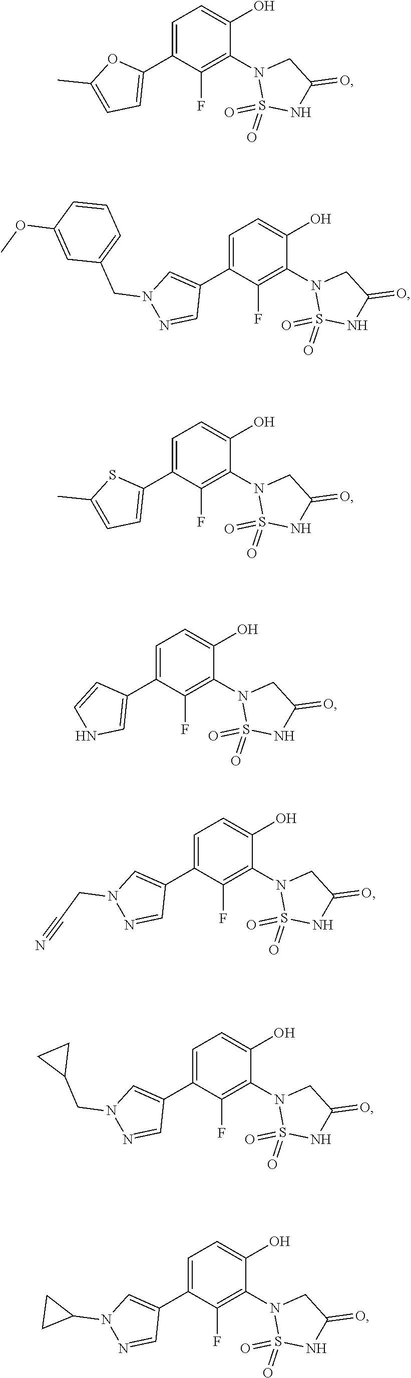 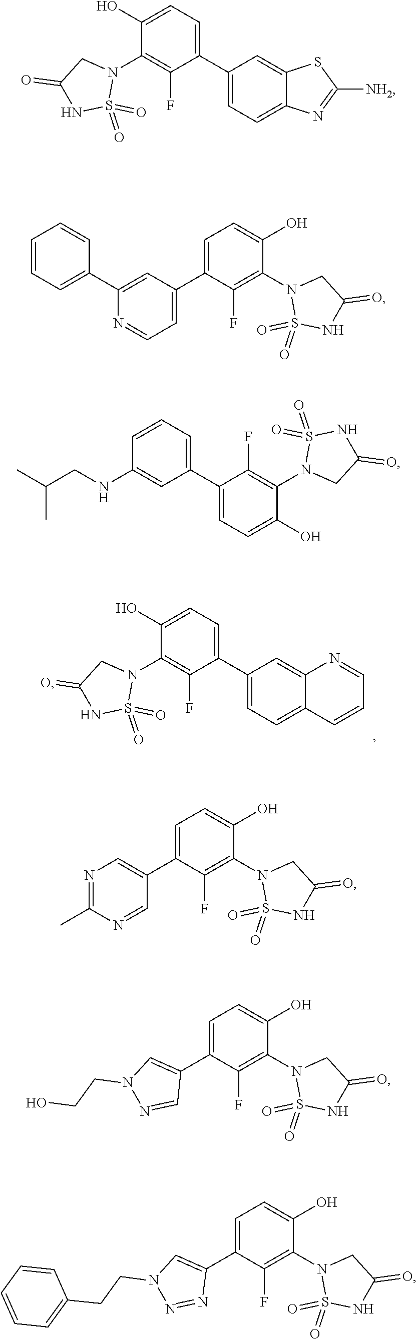 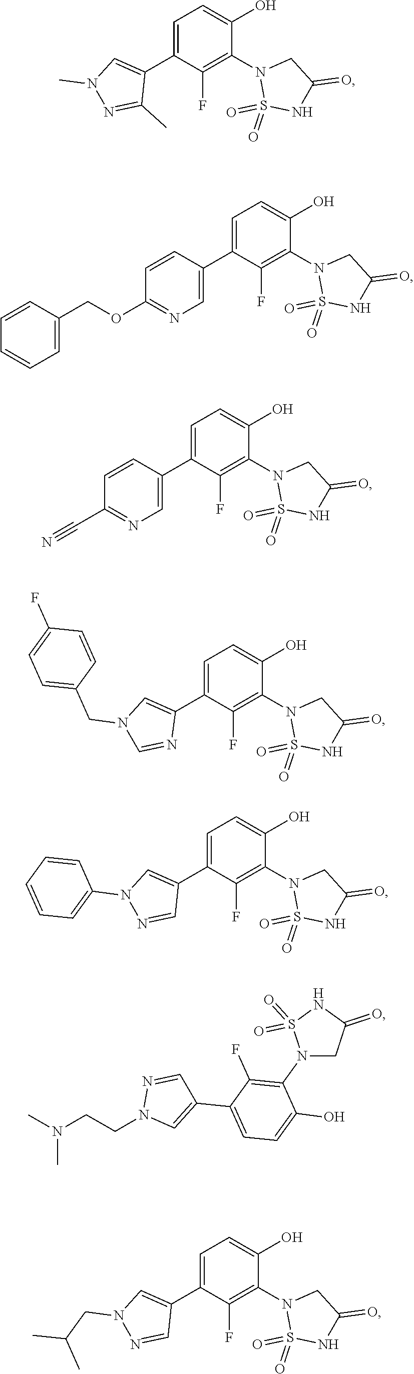 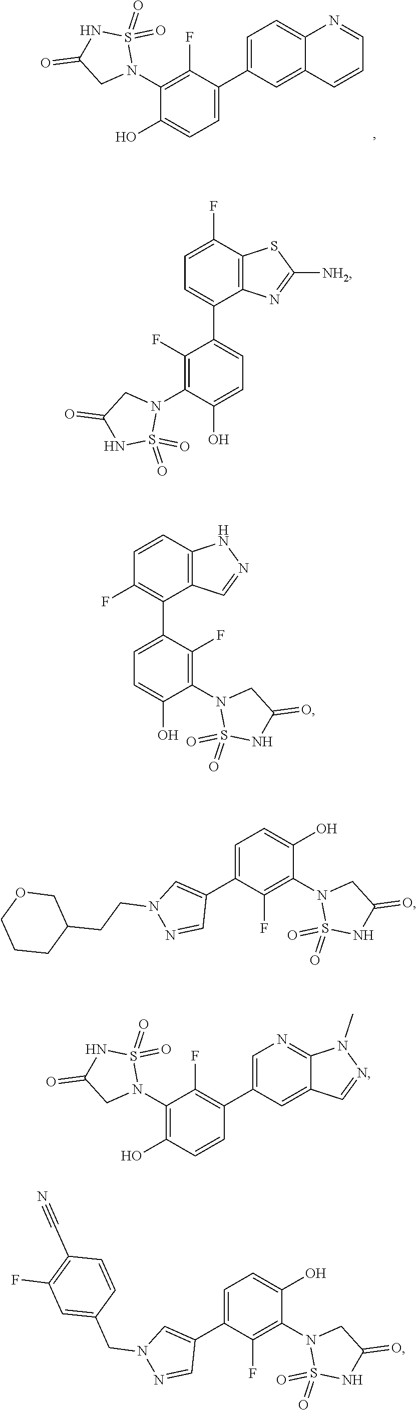 [SMILES: O=C1CN(c2c(O)ccc(-c3ccc4ncccc4c3)c2F)S(=O)(=O)N1 ; Nc1nc2c(-c3ccc(O)c(N4CC(=O)NS4(=O)=O)c3F)ccc(F)c2s1 ; O=C1CN(c2c(O)ccc(-c3c(F)ccc4[nH]ncc34)c2F)S(=O)(=O)N1 ; O=C1CN(c2c(O)ccc(-c3cnn(CCC4CCCOC4)c3)c2F)S(=O)(=O)N1 ; Cn1ncc2cc(-c3ccc(O)c(N4CC(=O)NS4(=O)=O)c3F)cnc21 ; N#Cc1ccc(Cn2cc(-c3ccc(O)c(N4CC(=O)NS4(=O)=O)c3F)cn2)cc1F] 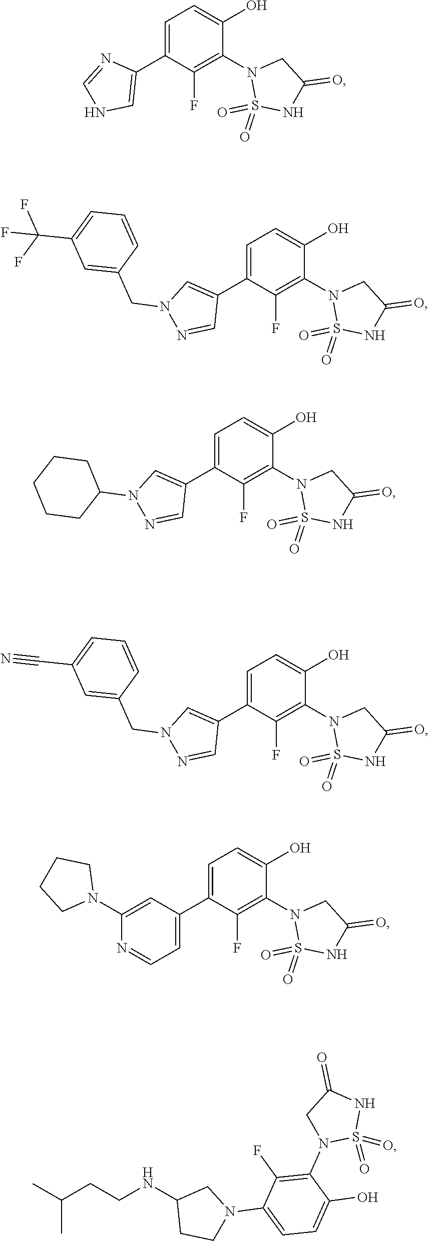 [SMILES: O=C1CN(c2c(O)ccc(-c3c[nH]cn3)c2F)S(=O)(=O)N1 ; O=C1CN(c2c(O)ccc(-c3cnn(Cc4cccc(C(F)(F)F)c4)c3)c2F)S(=O)(=O)N1 ; O=C1CN(c2c(O)ccc(-c3cnn(C4CCCCC4)c3)c2F)S(=O)(=O)N1 ; N#Cc1cccc(Cn2cc(-c3ccc(O)c(N4CC(=O)NS4(=O)=O)c3F)cn2)c1 ; O=C1CN(c2c(O)ccc(-c3ccnc(N4CCCC4)c3)c2F)S(=O)(=O)N1 ; CC(C)CCNC1CCN(c2ccc(O)c(N3CC(=O)NS3(=O)=O)c2F)C1] 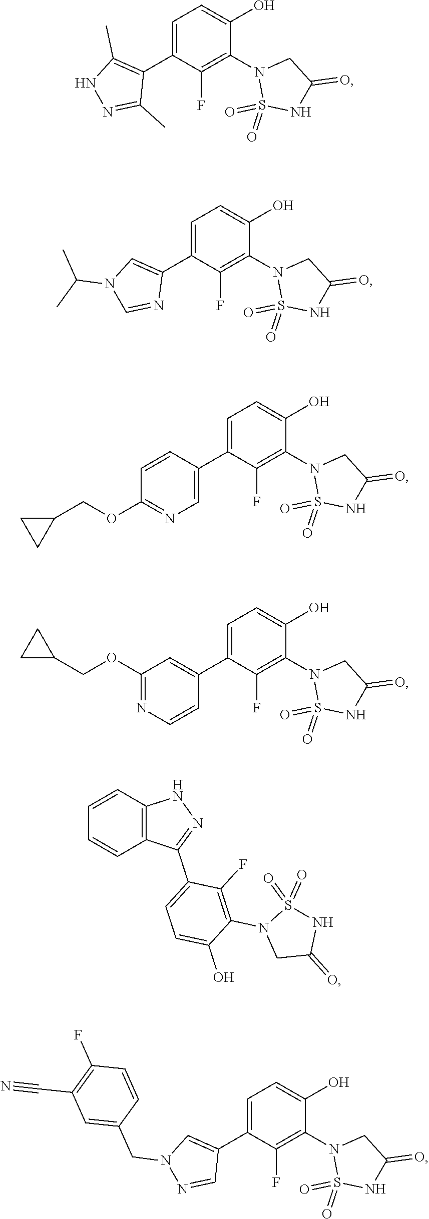 [SMILES: Cc1n[nH]c(C)c1-c1ccc(O)c(N2CC(=O)NS2(=O)=O)c1F ; CC(C)n1cnc(-c2ccc(O)c(N3CC(=O)NS3(=O)=O)c2F)c1 ; O=C1CN(c2c(O)ccc(-c3ccc(OCC4CC4)nc3)c2F)S(=O)(=O)N1 ; O=C1CN(c2c(O)ccc(-c3ccnc(OCC4CC4)c3)c2F)S(=O)(=O)N1 ; O=C1CN(c2c(O)ccc(-c3n[nH]c4ccccc34)c2F)S(=O)(=O)N1 ; N#Cc1cc(Cn2cc(-c3ccc(O)c(N4CC(=O)NS4(=O)=O)c3F)cn2)ccc1F] 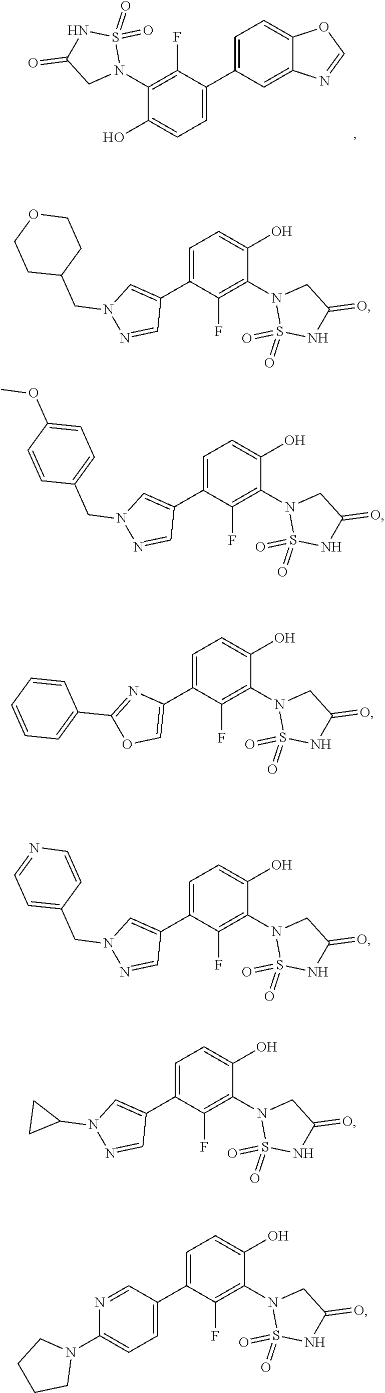 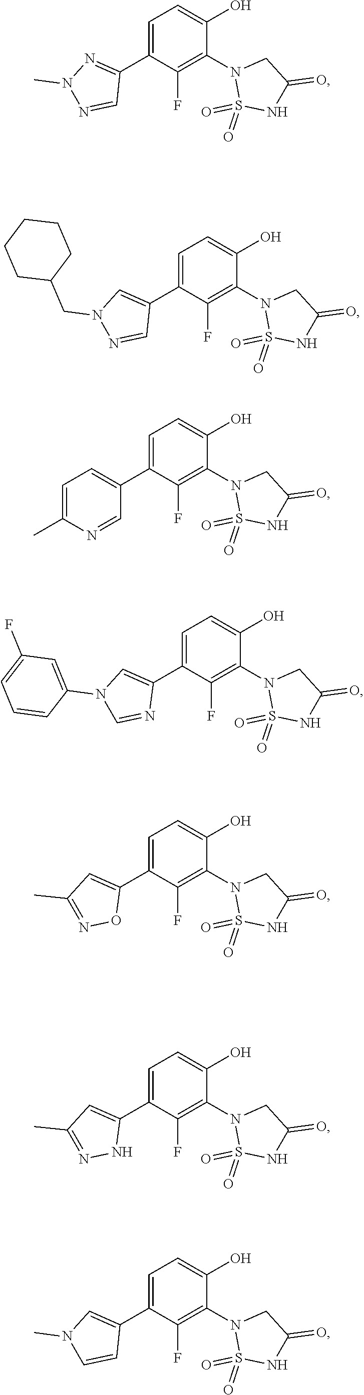 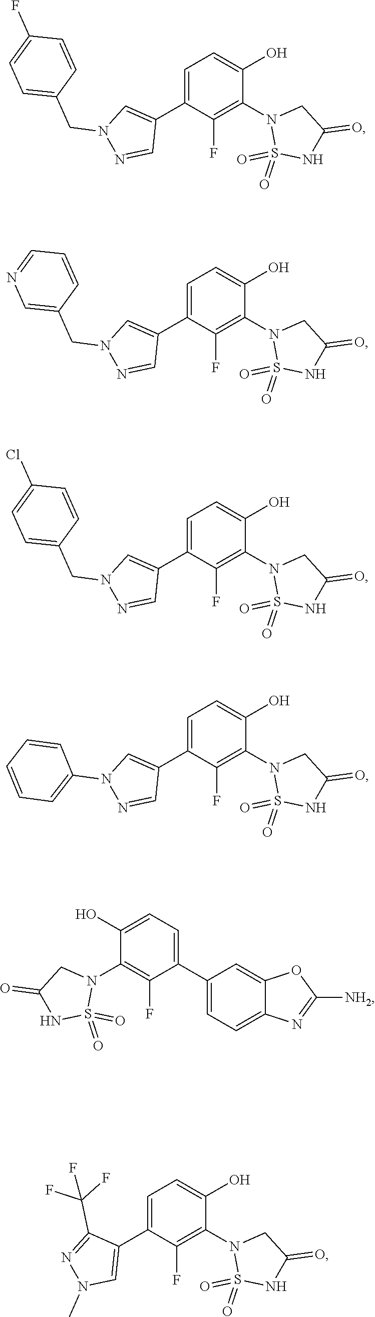 [SMILES: O=C1CN(c2c(O)ccc(-c3cnn(Cc4ccc(F)cc4)c3)c2F)S(=O)(=O)N1 ; O=C1CN(c2c(O)ccc(-c3cnn(Cc4cccnc4)c3)c2F)S(=O)(=O)N1 ; O=C1CN(c2c(O)ccc(-c3cnn(Cc4ccc(Cl)cc4)c3)c2F)S(=O)(=O)N1 ; O=C1CN(c2c(O)ccc(-c3cnn(-c4ccccc4)c3)c2F)S(=O)(=O)N1 ; Nc1nc2ccc(-c3ccc(O)c(N4CC(=O)NS4(=O)=O)c3F)cc2o1 ; Cn1cc(-c2ccc(O)c(N3CC(=O)NS3(=O)=O)c2F)c(C(F)(F)F)n1] 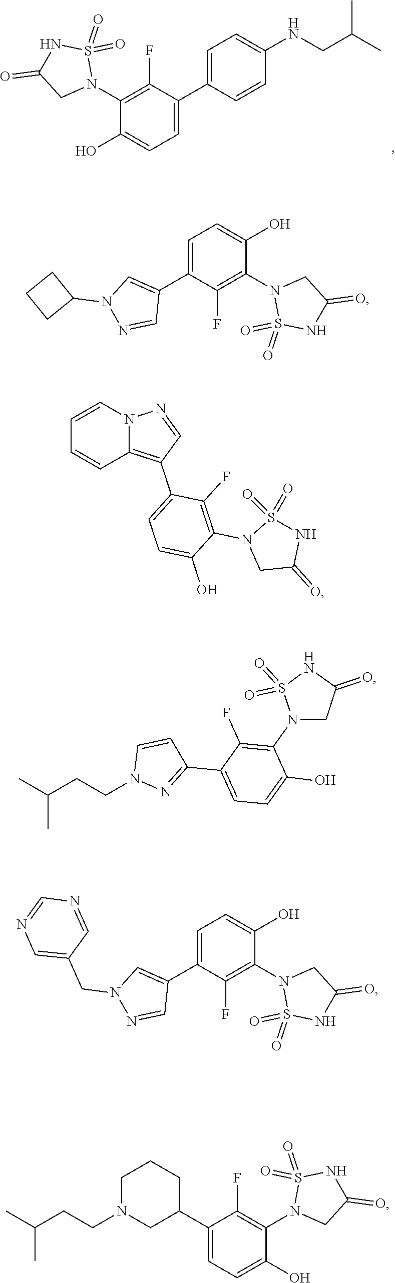 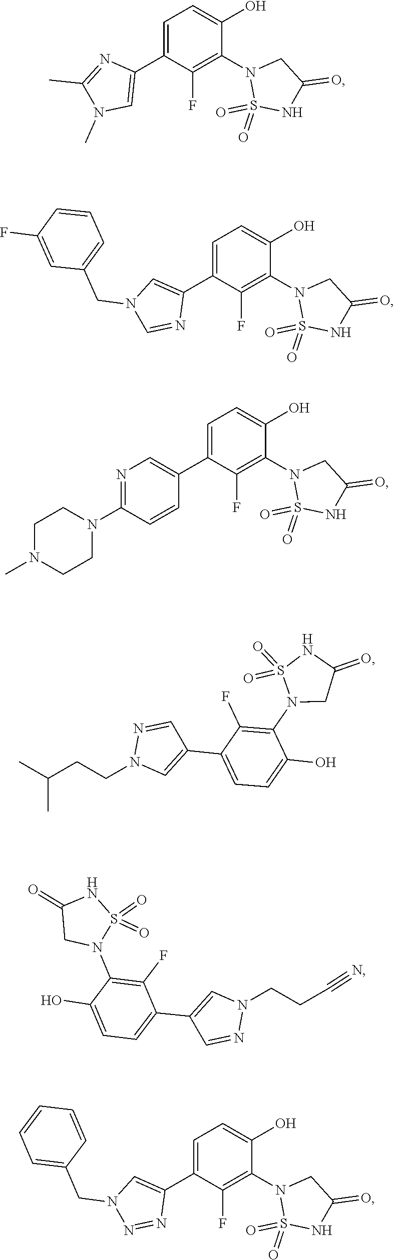 [SMILES: Cc1nc(-c2ccc(O)c(N3CC(=O)NS3(=O)=O)c2F)cn1C ; O=C1CN(c2c(O)ccc(-c3cn(Cc4cccc(F)c4)cn3)c2F)S(=O)(=O)N1 ; CN1CCN(c2ccc(-c3ccc(O)c(N4CC(=O)NS4(=O)=O)c3F)cn2)CC1 ; CC(C)CCn1cc(-c2ccc(O)c(N3CC(=O)NS3(=O)=O)c2F)cn1 ; N#CCCn1cc(-c2ccc(O)c(N3CC(=O)NS3(=O)=O)c2F)cn1 ; O=C1CN(c2c(O)ccc(-c3cn(Cc4ccccc4)nn3)c2F)S(=O)(=O)N1] 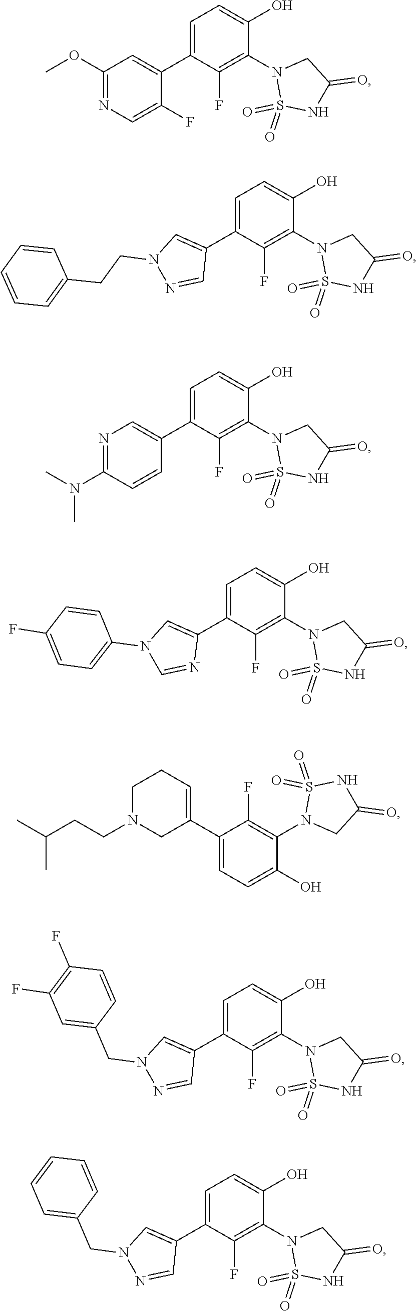 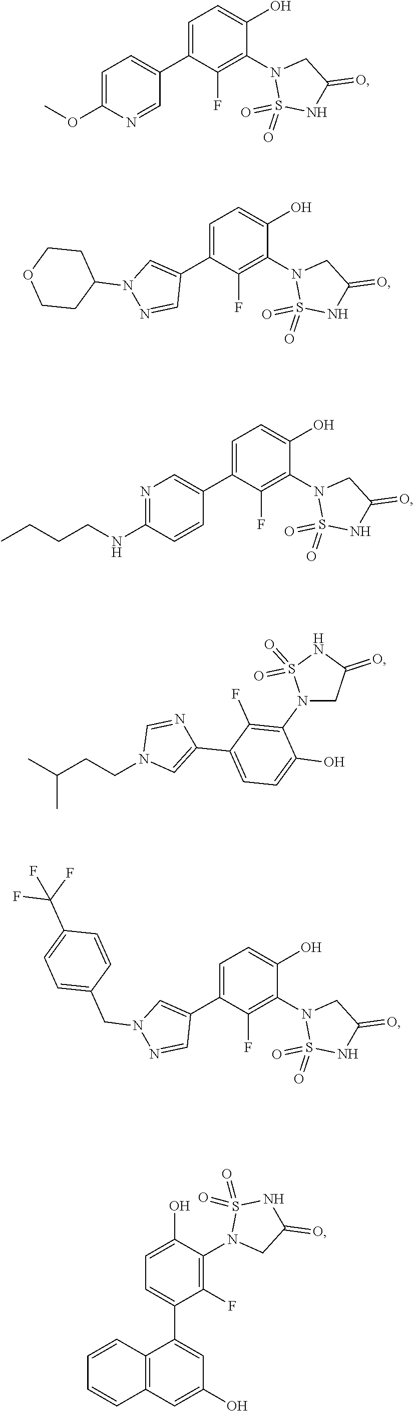 [SMILES: COc1ccc(-c2ccc(O)c(N3CC(=O)NS3(=O)=O)c2F)cn1 ; O=C1CN(c2c(O)ccc(-c3cnn(C4CCOCC4)c3)c2F)S(=O)(=O)N1 ; CCCCNc1ccc(-c2ccc(O)c(N3CC(=O)NS3(=O)=O)c2F)cn1 ; CC(C)CCn1cnc(-c2ccc(O)c(N3CC(=O)NS3(=O)=O)c2F)c1 ; O=C1CN(c2c(O)ccc(-c3cnn(Cc4ccc(C(F)(F)F)cc4)c3)c2F)S(=O)(=O)N1 ; O=C1CN(c2c(O)ccc(-c3cc(O)cc4ccccc34)c2F)S(=O)(=O)N1] 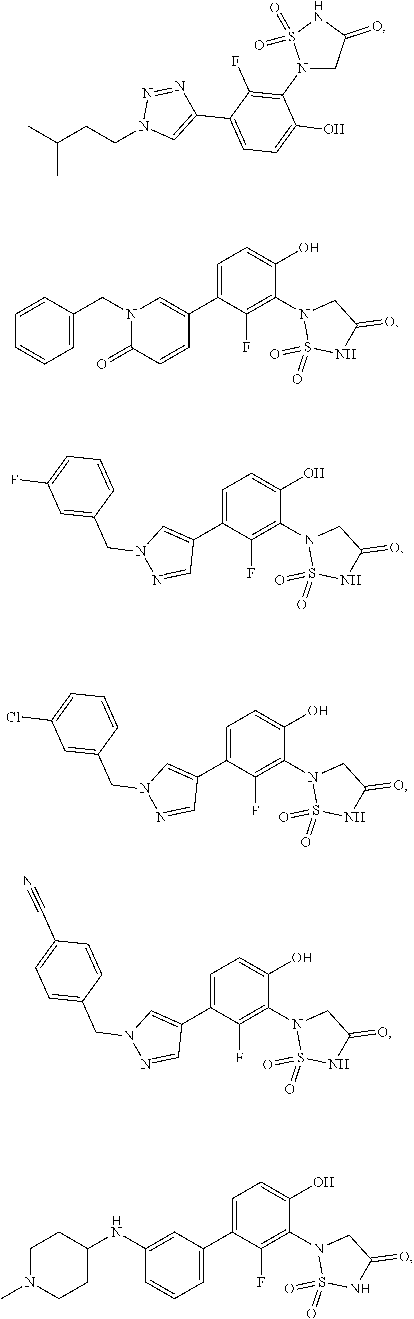 [SMILES: CC(C)CCn1cc(-c2ccc(O)c(N3CC(=O)NS3(=O)=O)c2F)nn1 ; O=C1CN(c2c(O)ccc(-c3ccc(=O)n(Cc4ccccc4)c3)c2F)S(=O)(=O)N1 ; O=C1CN(c2c(O)ccc(-c3cnn(Cc4cccc(F)c4)c3)c2F)S(=O)(=O)N1 ; O=C1CN(c2c(O)ccc(-c3cnn(Cc4cccc(Cl)c4)c3)c2F)S(=O)(=O)N1 ; N#Cc1ccc(Cn2cc(-c3ccc(O)c(N4CC(=O)NS4(=O)=O)c3F)cn2)cc1 ; CN1CCC(Nc2cccc(-c3ccc(O)c(N4CC(=O)NS4(=O)=O)c3F)c2)CC1] 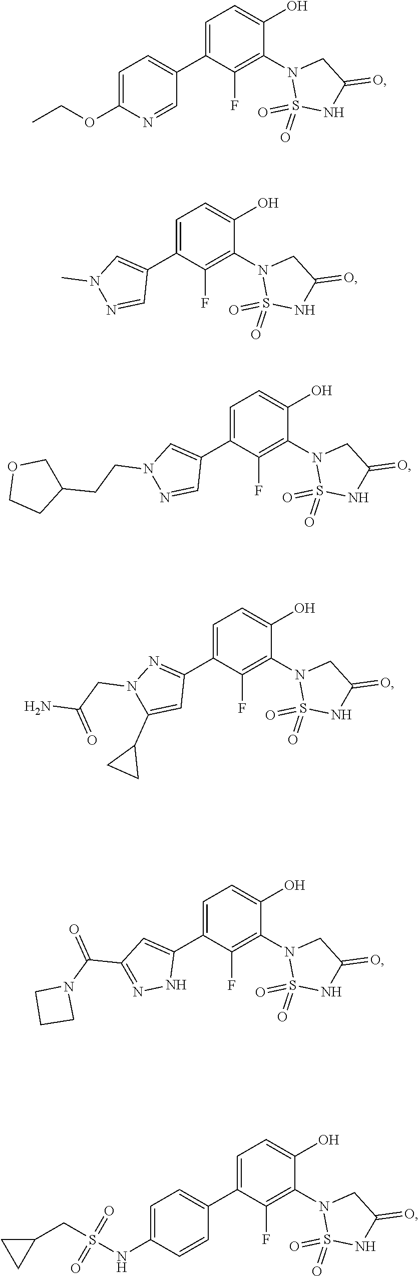 [SMILES: CCOc1ccc(-c2ccc(O)c(N3CC(=O)NS3(=O)=O)c2F)cn1 ; Cn1cc(-c2ccc(O)c(N3CC(=O)NS3(=O)=O)c2F)cn1 ; O=C1CN(c2c(O)ccc(-c3cnn(CCC4CCOC4)c3)c2F)S(=O)(=O)N1 ; NC(=O)Cn1nc(-c2ccc(O)c(N3CC(=O)NS3(=O)=O)c2F)cc1C1CC1 ; O=C1CN(c2c(O)ccc(-c3cc(C(=O)N4CCC4)n[nH]3)c2F)S(=O)(=O)N1 ; O=C1CN(c2c(O)ccc(-c3ccc(NS(=O)(=O)CC4CC4)cc3)c2F)S(=O)(=O)N1] 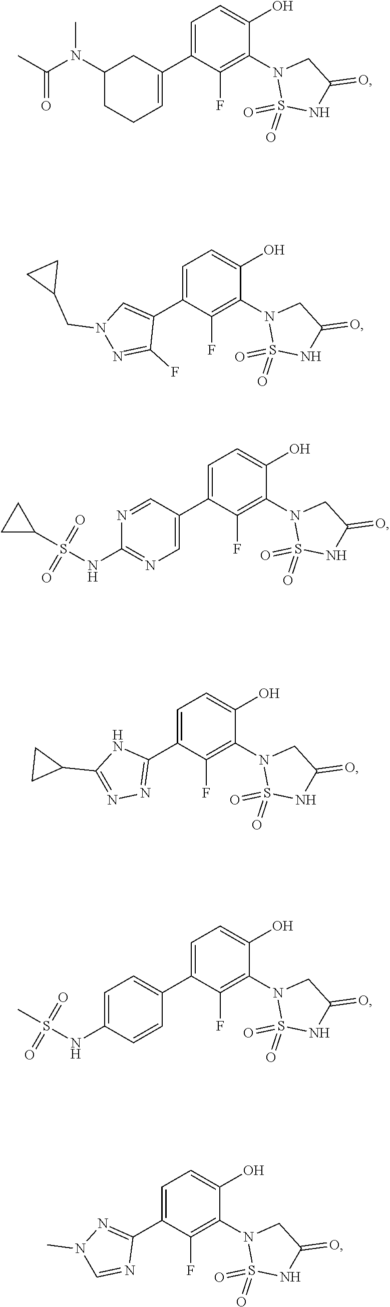 [SMILES: CC(=O)N(C)C1CCC=C(c2ccc(O)c(N3CC(=O)NS3(=O)=O)c2F)C1 ; O=C1CN(c2c(O)ccc(-c3cn(CC4CC4)nc3F)c2F)S(=O)(=O)N1 ; O=C1CN(c2c(O)ccc(-c3cnc(NS(=O)(=O)C4CC4)nc3)c2F)S(=O)(=O)N1 ; O=C1CN(c2c(O)ccc(-c3nnc(C4CC4)[nH]3)c2F)S(=O)(=O)N1 ; CS(=O)(=O)Nc1ccc(-c2ccc(O)c(N3CC(=O)NS3(=O)=O)c2F)cc1 ; Cn1cnc(-c2ccc(O)c(N3CC(=O)NS3(=O)=O)c2F)n1] 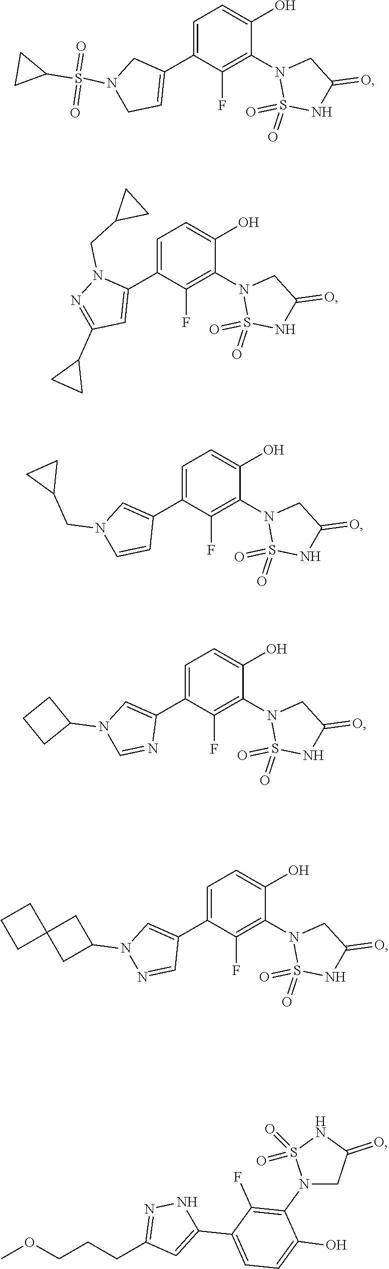 [SMILES: O=C1CN(c2c(O)ccc(C3=CCN(S(=O)(=O)C4CC4)C3)c2F)S(=O)(=O)N1 ; O=C1CN(c2c(O)ccc(-c3cc(C4CC4)nn3CC3CC3)c2F)S(=O)(=O)N1 ; O=C1CN(c2c(O)ccc(-c3ccn(CC4CC4)c3)c2F)S(=O)(=O)N1 ; O=C1CN(c2c(O)ccc(-c3cn(C4CCC4)cn3)c2F)S(=O)(=O)N1 ; O=C1CN(c2c(O)ccc(-c3cnn(C4CC5(CCC5)C4)c3)c2F)S(=O)(=O)N1 ; COCCCc1cc(-c2ccc(O)c(N3CC(=O)NS3(=O)=O)c2F)[nH]n1] 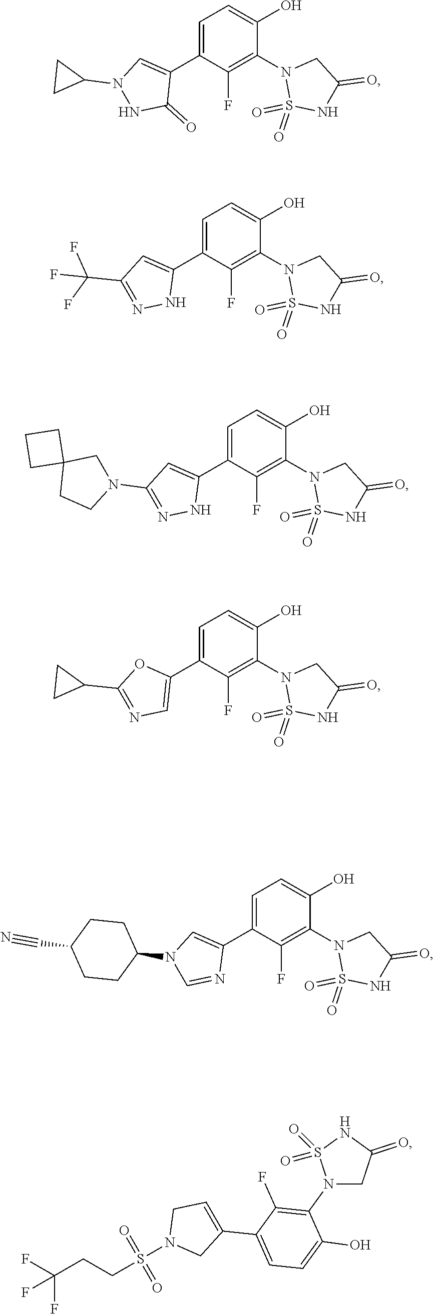 [SMILES: O=C1CN(c2c(O)ccc(-c3cn(C4CC4)[nH]c3=O)c2F)S(=O)(=O)N1 ; O=C1CN(c2c(O)ccc(-c3cc(C(F)(F)F)n[nH]3)c2F)S(=O)(=O)N1 ; O=C1CN(c2c(O)ccc(-c3cc(N4CCC5(CCC5)C4)n[nH]3)c2F)S(=O)(=O)N1 ; O=C1CN(c2c(O)ccc(-c3cnc(C4CC4)o3)c2F)S(=O)(=O)N1 ; N#C[C@H]1CC[C@H](n2cnc(-c3ccc(O)c(N4CC(=O)NS4(=O)=O)c3F)c2)CC1 ; O=C1CN(c2c(O)ccc(C3=CCN(S(=O)(=O)CCC(F)(F)F)C3)c2F)S(=O)(=O)N1] 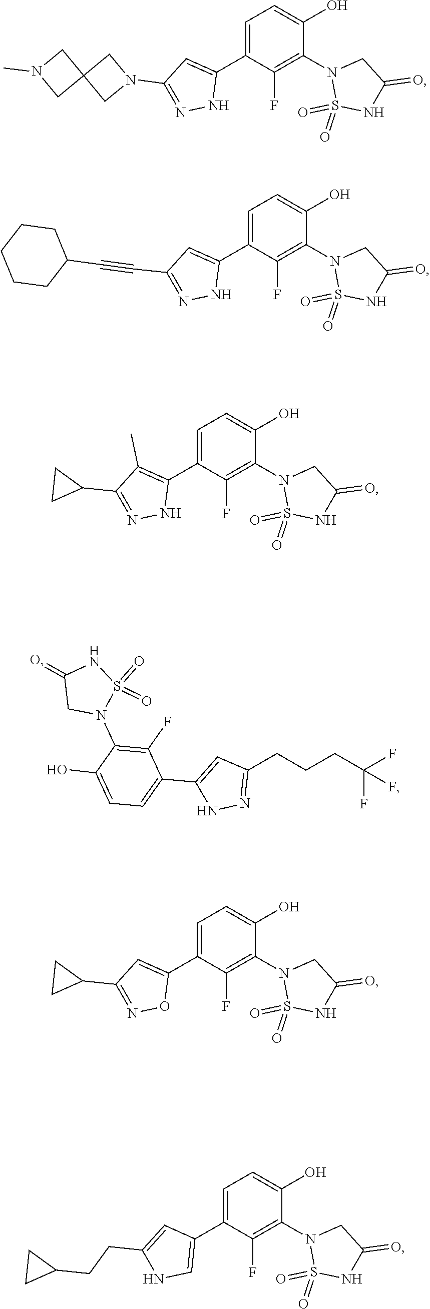 [SMILES: CN1CC2(C1)CN(c1cc(-c3ccc(O)c(N4CC(=O)NS4(=O)=O)c3F)[nH]n1)C2 ; O=C1CN(c2c(O)ccc(-c3cc(C#CC4CCCCC4)n[nH]3)c2F)S(=O)(=O)N1 ; Cc1c(C2CC2)n[nH]c1-c1ccc(O)c(N2CC(=O)NS2(=O)=O)c1F ; O=C1CN(c2c(O)ccc(-c3cc(CCCC(F)(F)F)n[nH]3)c2F)S(=O)(=O)N1 ; O=C1CN(c2c(O)ccc(-c3cc(C4CC4)no3)c2F)S(=O)(=O)N1 ; O=C1CN(c2c(O)ccc(-c3c[nH]c(CCC4CC4)c3)c2F)S(=O)(=O)N1] 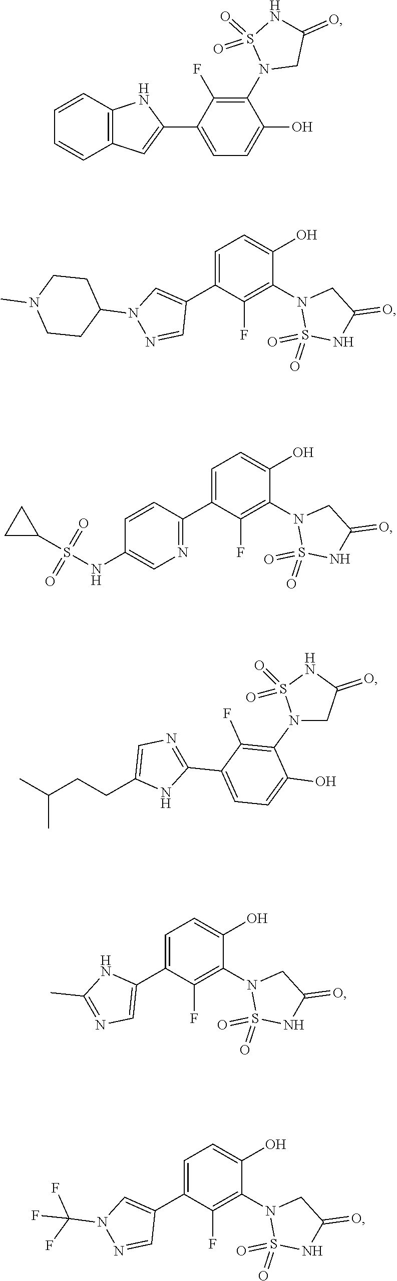 [SMILES: O=C1CN(c2c(O)ccc(-c3cc4ccccc4[nH]3)c2F)S(=O)(=O)N1 ; CN1CCC(n2cc(-c3ccc(O)c(N4CC(=O)NS4(=O)=O)c3F)cn2)CC1 ; O=C1CN(c2c(O)ccc(-c3ccc(NS(=O)(=O)C4CC4)cn3)c2F)S(=O)(=O)N1 ; CC(C)CCc1cnc(-c2ccc(O)c(N3CC(=O)NS3(=O)=O)c2F)[nH]1 ; Cc1ncc(-c2ccc(O)c(N3CC(=O)NS3(=O)=O)c2F)[nH]1 ; O=C1CN(c2c(O)ccc(-c3cnn(C(F)(F)F)c3)c2F)S(=O)(=O)N1] 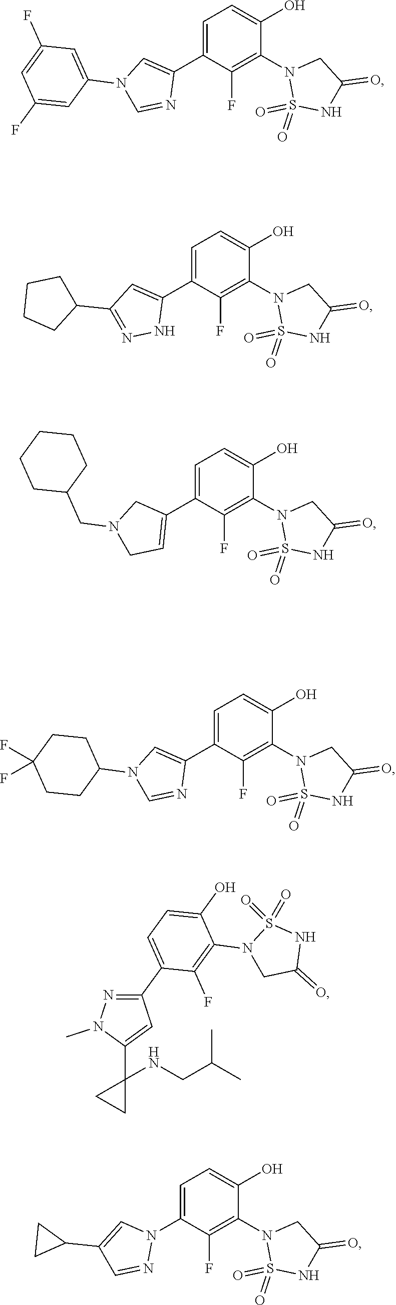 [SMILES: O=C1CN(c2c(O)ccc(-c3cn(-c4cc(F)cc(F)c4)cn3)c2F)S(=O)(=O)N1 ; O=C1CN(c2c(O)ccc(-c3cc(C4CCCC4)n[nH]3)c2F)S(=O)(=O)N1 ; O=C1CN(c2c(O)ccc(C3=CCN(CC4CCCCC4)C3)c2F)S(=O)(=O)N1 ; O=C1CN(c2c(O)ccc(-c3cn(C4CCC(F)(F)CC4)cn3)c2F)S(=O)(=O)N1 ; CC(C)CNC1(c2cc(-c3ccc(O)c(N4CC(=O)NS4(=O)=O)c3F)nn2C)CC1 ; O=C1CN(c2c(O)ccc(-n3cc(C4CC4)cn3)c2F)S(=O)(=O)N1] 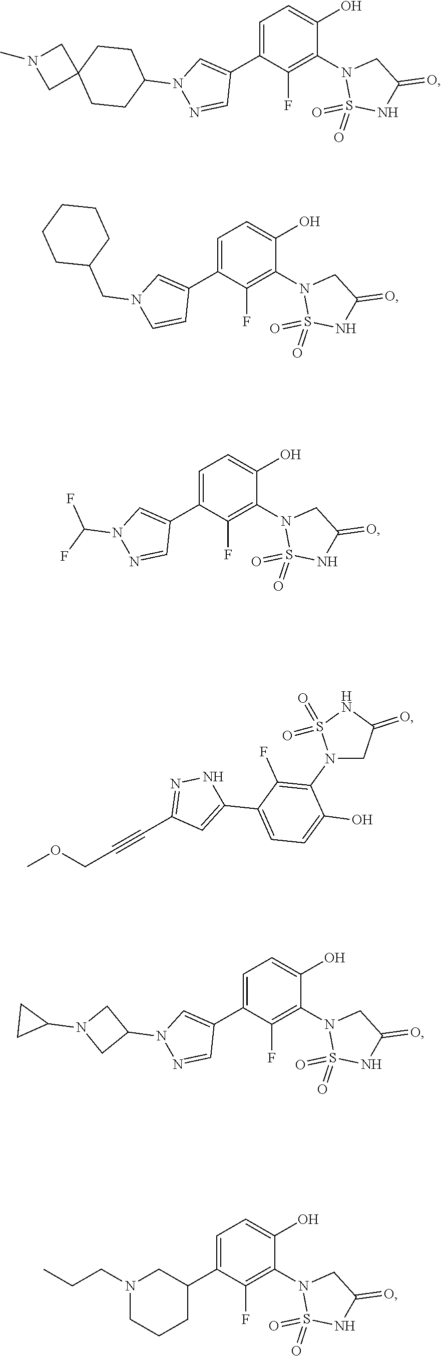 [SMILES: CN1CC2(CCC(n3cc(-c4ccc(O)c(N5CC(=O)NS5(=O)=O)c4F)cn3)CC2)C1 ; O=C1CN(c2c(O)ccc(-c3ccn(CC4CCCCC4)c3)c2F)S(=O)(=O)N1 ; O=C1CN(c2c(O)ccc(-c3cnn(C(F)F)c3)c2F)S(=O)(=O)N1 ; COCC#Cc1cc(-c2ccc(O)c(N3CC(=O)NS3(=O)=O)c2F)[nH]n1 ; O=C1CN(c2c(O)ccc(-c3cnn(C4CN(C5CC5)C4)c3)c2F)S(=O)(=O)N1 ; CCCN1CCCC(c2ccc(O)c(N3CC(=O)NS3(=O)=O)c2F)C1] 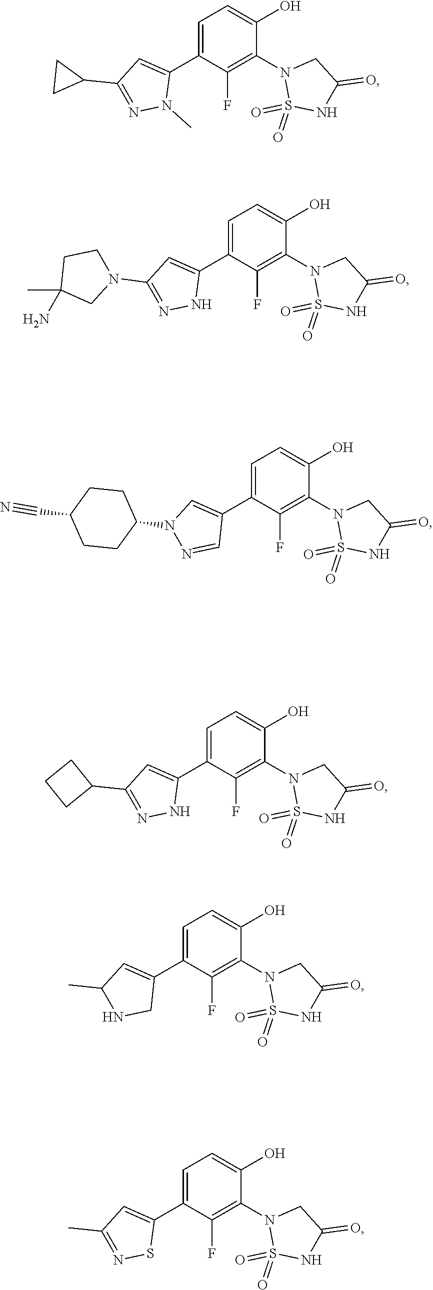 [SMILES: Cn1nc(C2CC2)cc1-c1ccc(O)c(N2CC(=O)NS2(=O)=O)c1F ; CC1(N)CCN(c2cc(-c3ccc(O)c(N4CC(=O)NS4(=O)=O)c3F)[nH]n2)C1 ; N#C[C@H]1CC[C@@H](n2cc(-c3ccc(O)c(N4CC(=O)NS4(=O)=O)c3F)cn2)CC1 ; O=C1CN(c2c(O)ccc(-c3cc(C4CCC4)n[nH]3)c2F)S(=O)(=O)N1 ; CC1C=C(c2ccc(O)c(N3CC(=O)NS3(=O)=O)c2F)CN1 ; Cc1cc(-c2ccc(O)c(N3CC(=O)NS3(=O)=O)c2F)sn1] 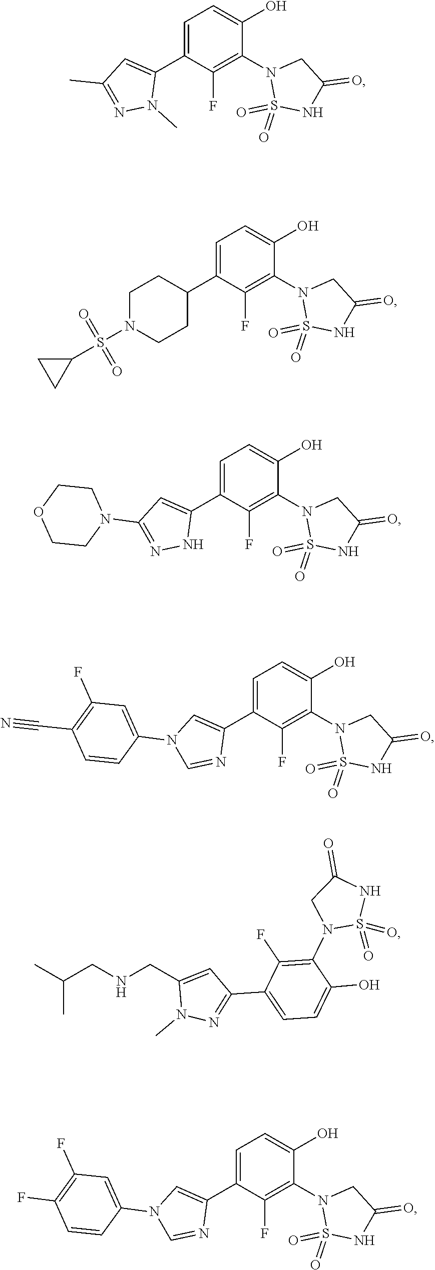 [SMILES: Cc1cc(-c2ccc(O)c(N3CC(=O)NS3(=O)=O)c2F)n(C)n1 ; O=C1CN(c2c(O)ccc(C3CCN(S(=O)(=O)C4CC4)CC3)c2F)S(=O)(=O)N1 ; O=C1CN(c2c(O)ccc(-c3cc(N4CCOCC4)n[nH]3)c2F)S(=O)(=O)N1 ; N#Cc1ccc(-n2cnc(-c3ccc(O)c(N4CC(=O)NS4(=O)=O)c3F)c2)cc1F ; CC(C)CNCc1cc(-c2ccc(O)c(N3CC(=O)NS3(=O)=O)c2F)nn1C ; O=C1CN(c2c(O)ccc(-c3cn(-c4ccc(F)c(F)c4)cn3)c2F)S(=O)(=O)N1] 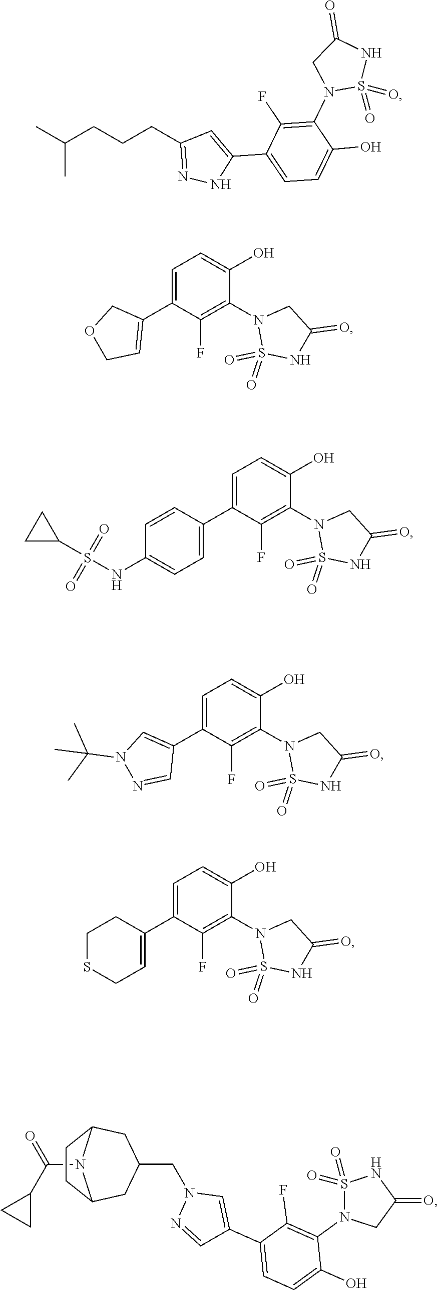 [SMILES: CC(C)CCCc1cc(-c2ccc(O)c(N3CC(=O)NS3(=O)=O)c2F)[nH]n1 ; O=C1CN(c2c(O)ccc(C3=CCOC3)c2F)S(=O)(=O)N1 ; O=C1CN(c2c(O)ccc(-c3ccc(NS(=O)(=O)C4CC4)cc3)c2F)S(=O)(=O)N1 ; CC(C)(C)n1cc(-c2ccc(O)c(N3CC(=O)NS3(=O)=O)c2F)cn1 ; O=C1CN(c2c(O)ccc(C3=CCSCC3)c2F)S(=O)(=O)N1 ; O=C1CN(c2c(O)ccc(-c3cnn(CC4CC5CCC(C4)N5C(=O)C4CC4)c3)c2F)S(=O)(=O)N1] 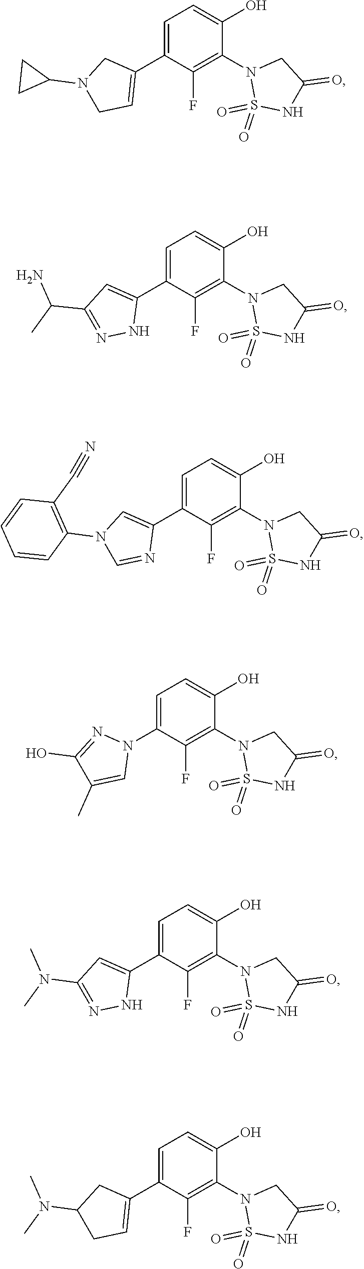 [SMILES: O=C1CN(c2c(O)ccc(C3=CCN(C4CC4)C3)c2F)S(=O)(=O)N1 ; CC(N)c1cc(-c2ccc(O)c(N3CC(=O)NS3(=O)=O)c2F)[nH]n1 ; N#Cc1ccccc1-n1cnc(-c2ccc(O)c(N3CC(=O)NS3(=O)=O)c2F)c1 ; Cc1cn(-c2ccc(O)c(N3CC(=O)NS3(=O)=O)c2F)nc1O ; CN(C)c1cc(-c2ccc(O)c(N3CC(=O)NS3(=O)=O)c2F)[nH]n1 ; CN(C)C1CC=C(c2ccc(O)c(N3CC(=O)NS3(=O)=O)c2F)C1] 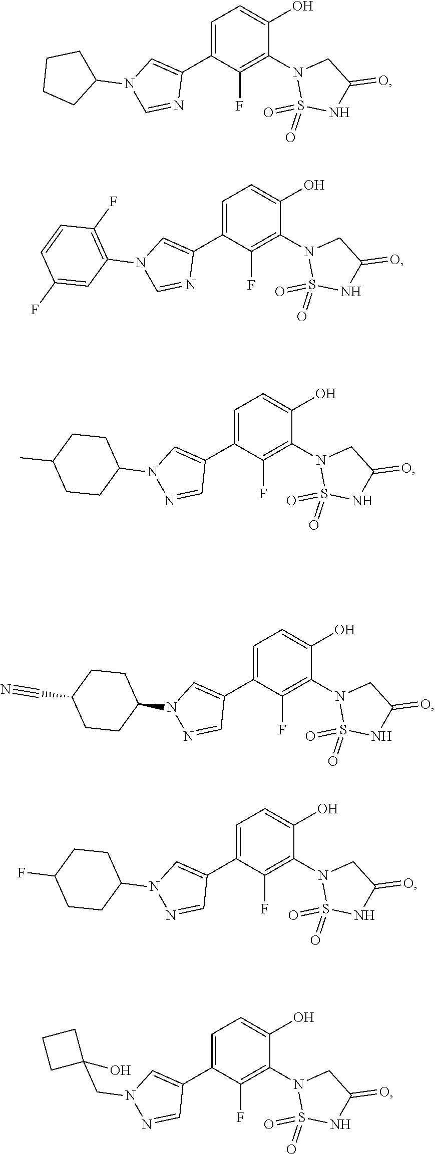 [SMILES: O=C1CN(c2c(O)ccc(-c3cn(C4CCCC4)cn3)c2F)S(=O)(=O)N1 ; O=C1CN(c2c(O)ccc(-c3cn(-c4cc(F)ccc4F)cn3)c2F)S(=O)(=O)N1 ; CC1CCC(n2cc(-c3ccc(O)c(N4CC(=O)NS4(=O)=O)c3F)cn2)CC1 ; N#C[C@H]1CC[C@H](n2cc(-c3ccc(O)c(N4CC(=O)NS4(=O)=O)c3F)cn2)CC1 ; O=C1CN(c2c(O)ccc(-c3cnn(C4CCC(F)CC4)c3)c2F)S(=O)(=O)N1 ; O=C1CN(c2c(O)ccc(-c3cnn(CC4(O)CCC4)c3)c2F)S(=O)(=O)N1] 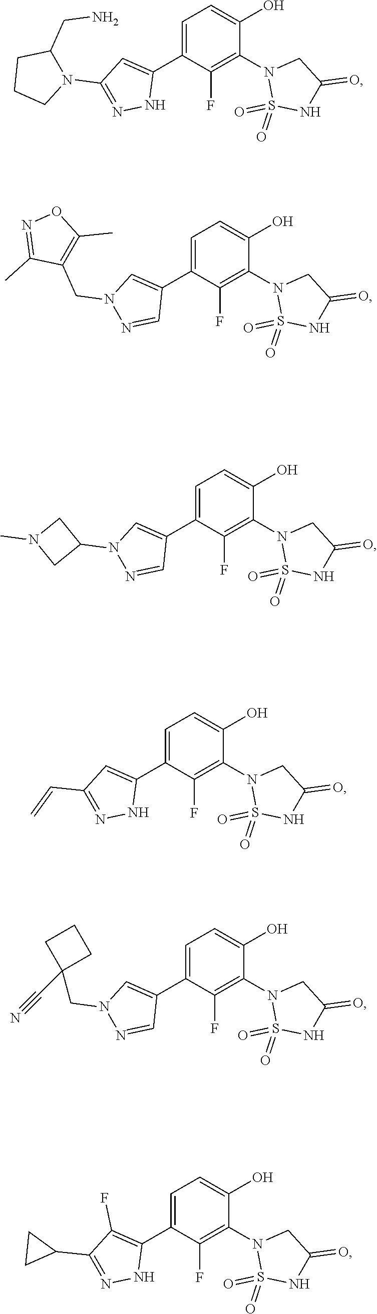 [SMILES: NCC1CCCN1c1cc(-c2ccc(O)c(N3CC(=O)NS3(=O)=O)c2F)[nH]n1 ; Cc1noc(C)c1Cn1cc(-c2ccc(O)c(N3CC(=O)NS3(=O)=O)c2F)cn1 ; CN1CC(n2cc(-c3ccc(O)c(N4CC(=O)NS4(=O)=O)c3F)cn2)C1 ; C=Cc1cc(-c2ccc(O)c(N3CC(=O)NS3(=O)=O)c2F)[nH]n1 ; N#CC1(Cn2cc(-c3ccc(O)c(N4CC(=O)NS4(=O)=O)c3F)cn2)CCC1 ; O=C1CN(c2c(O)ccc(-c3[nH]nc(C4CC4)c3F)c2F)S(=O)(=O)N1] 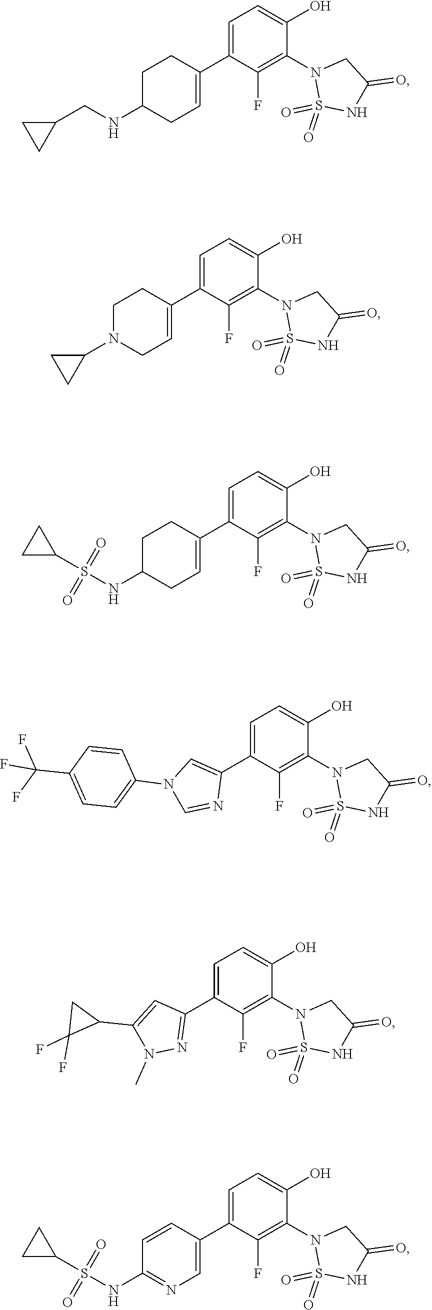 [SMILES: O=C1CN(c2c(O)ccc(C3=CCC(NCC4CC4)CC3)c2F)S(=O)(=O)N1 ; O=C1CN(c2c(O)ccc(C3=CCN(C4CC4)CC3)c2F)S(=O)(=O)N1 ; O=C1CN(c2c(O)ccc(C3=CCC(NS(=O)(=O)C4CC4)CC3)c2F)S(=O)(=O)N1 ; O=C1CN(c2c(O)ccc(-c3cn(-c4ccc(C(F)(F)F)cc4)cn3)c2F)S(=O)(=O)N1 ; Cn1nc(-c2ccc(O)c(N3CC(=O)NS3(=O)=O)c2F)cc1C1CC1(F)F ; O=C1CN(c2c(O)ccc(-c3ccc(NS(=O)(=O)C4CC4)nc3)c2F)S(=O)(=O)N1] 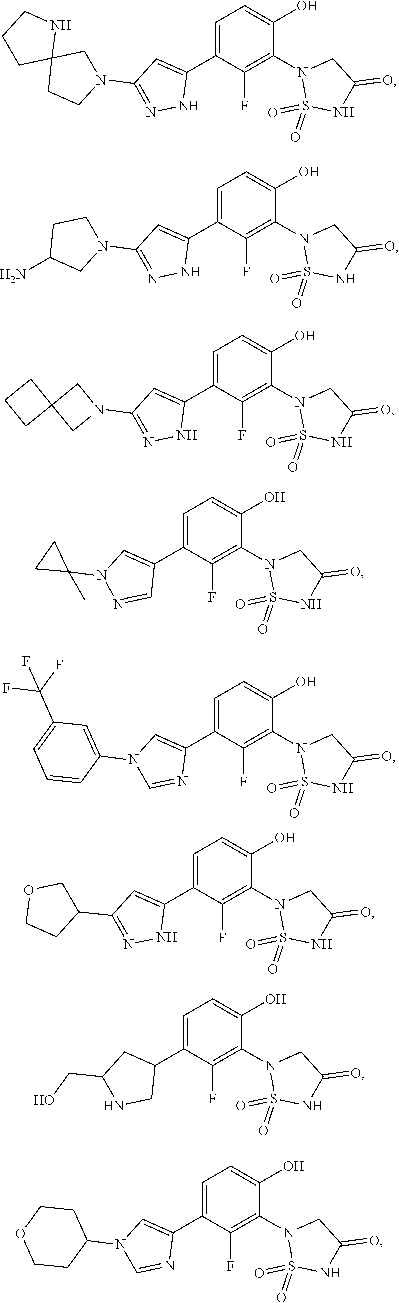 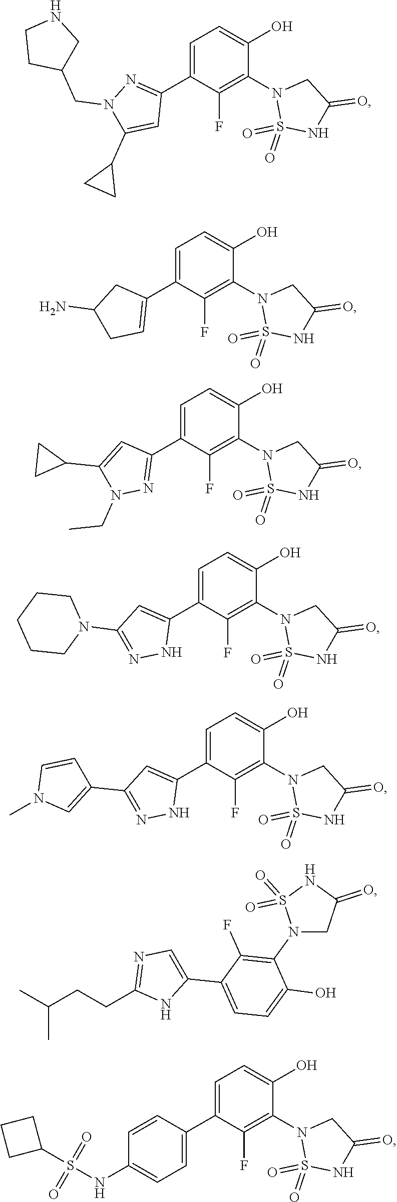 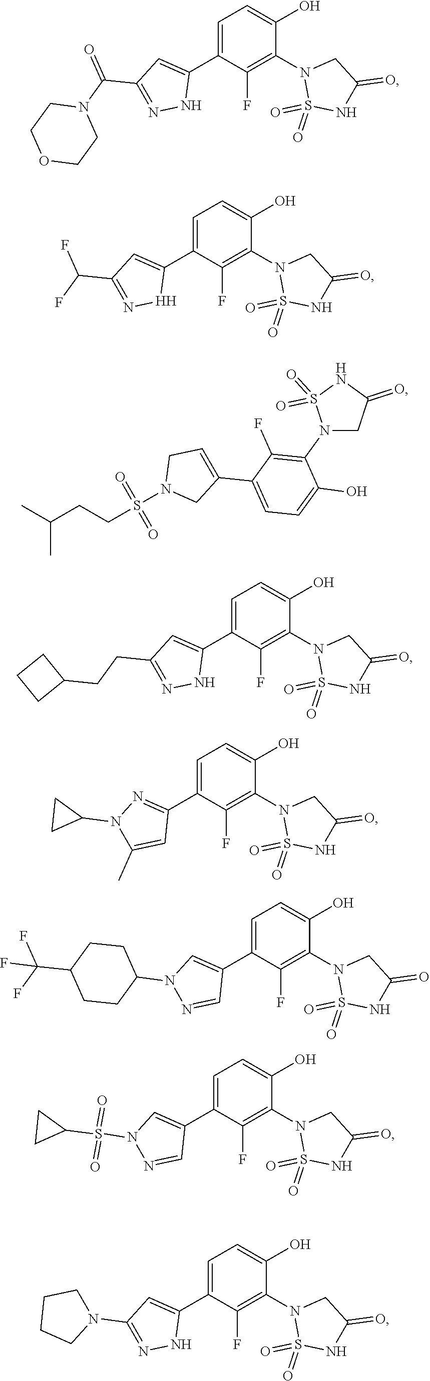 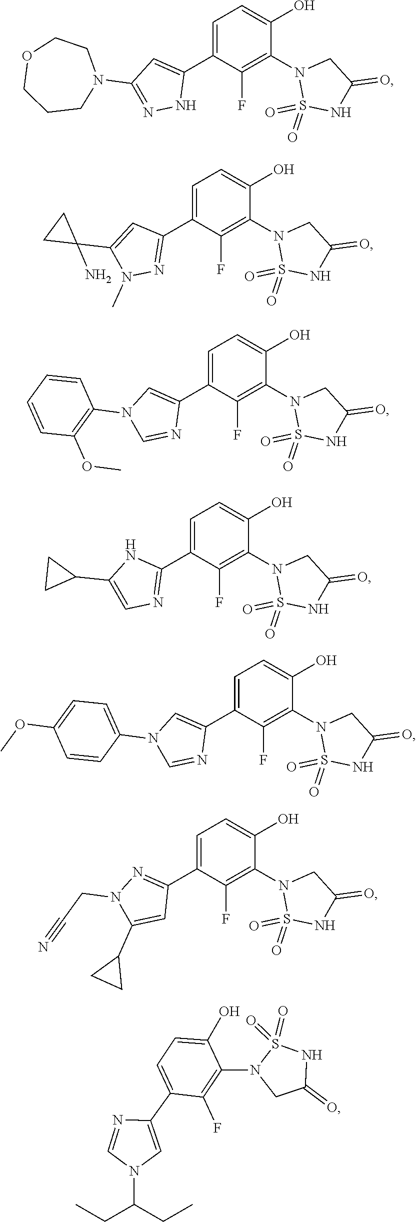 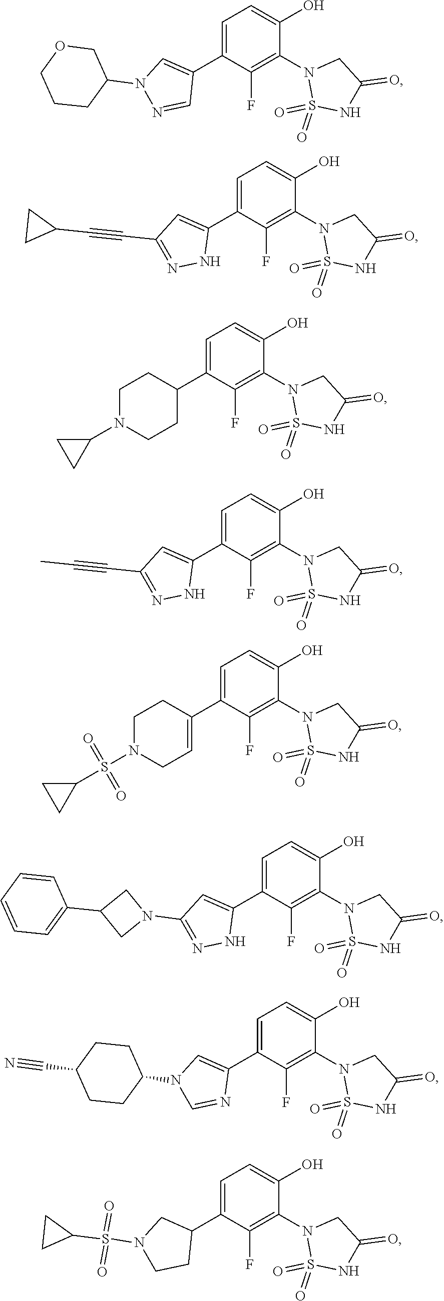 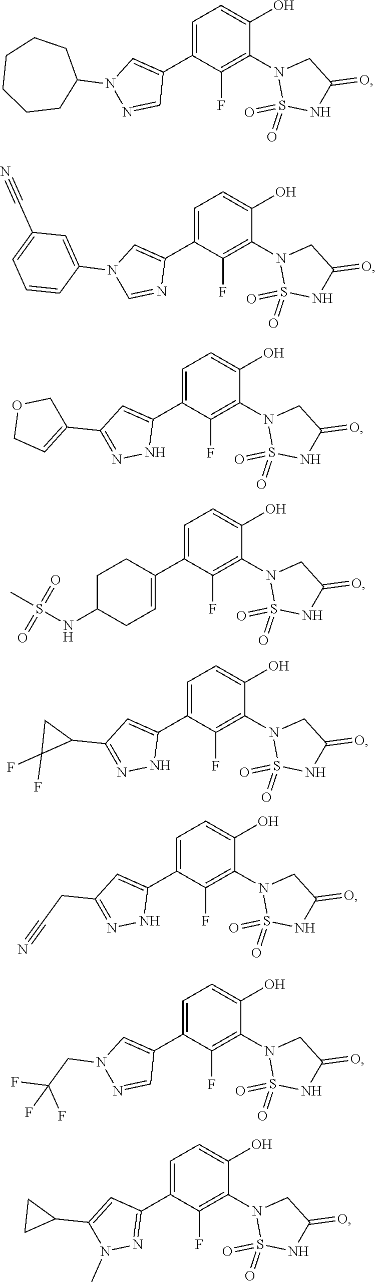 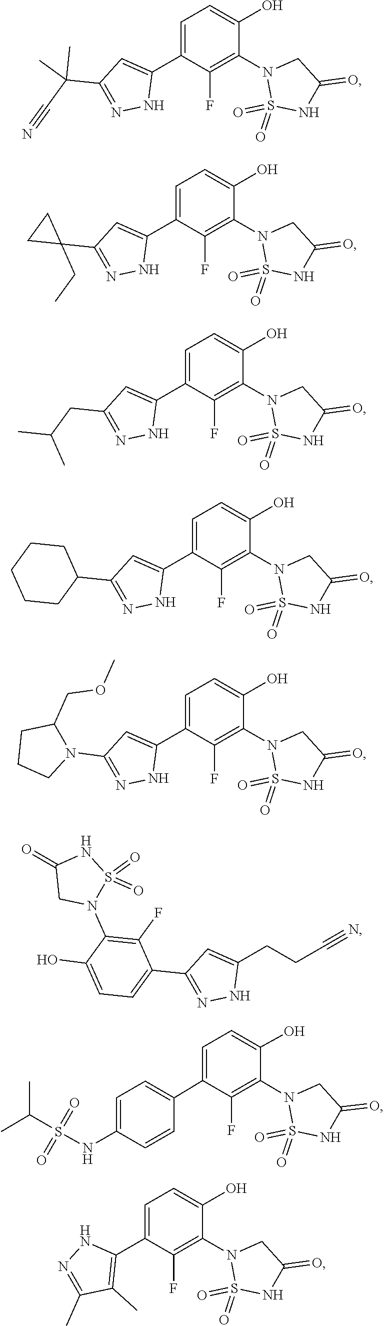 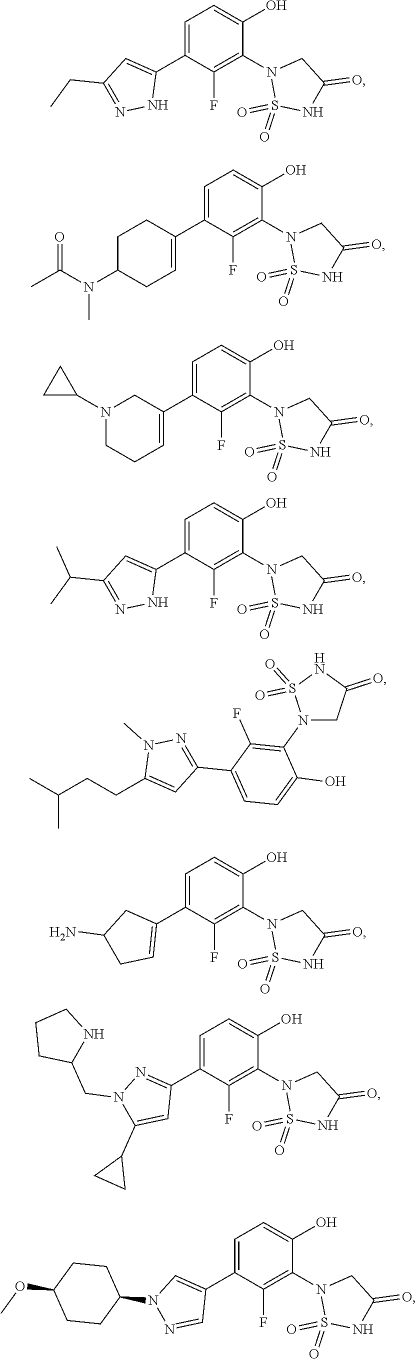 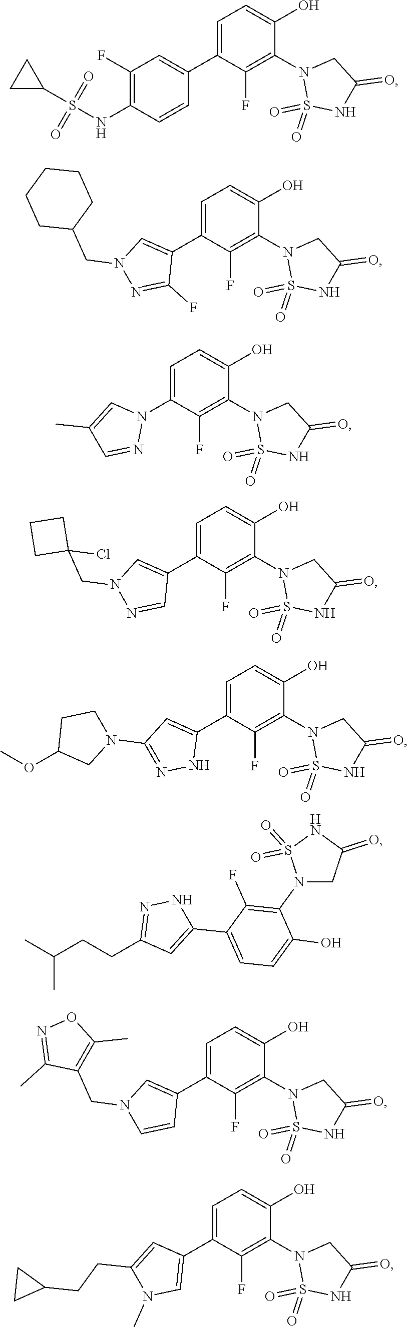 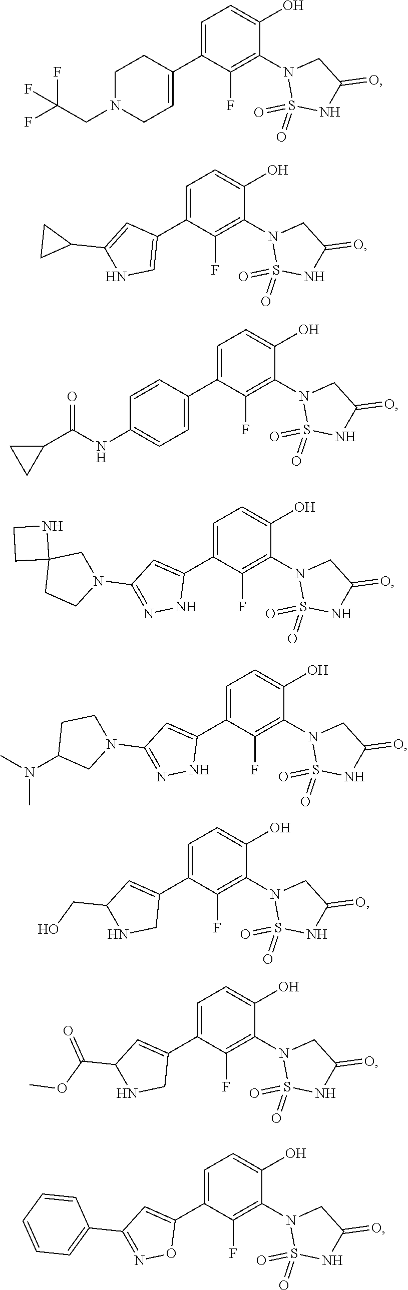 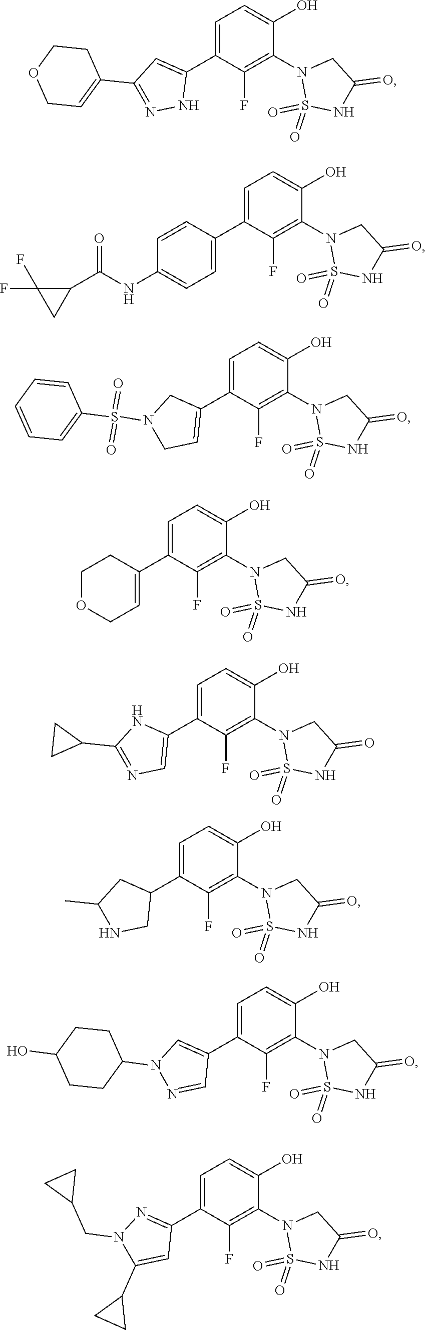 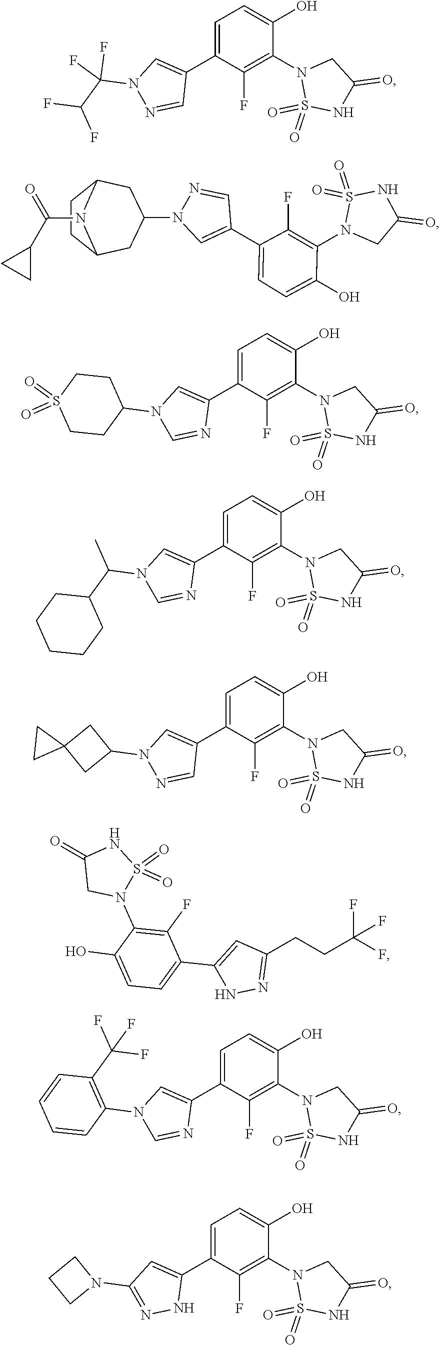 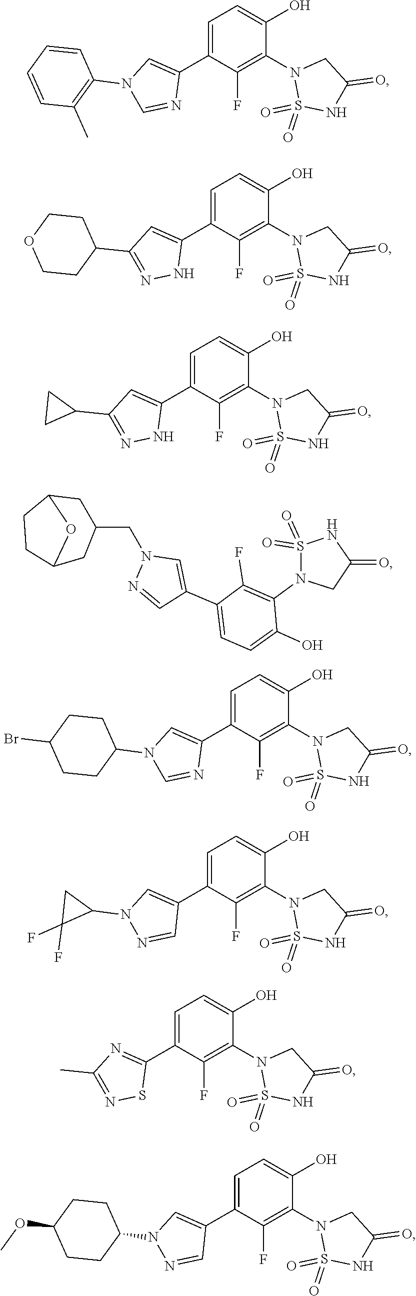 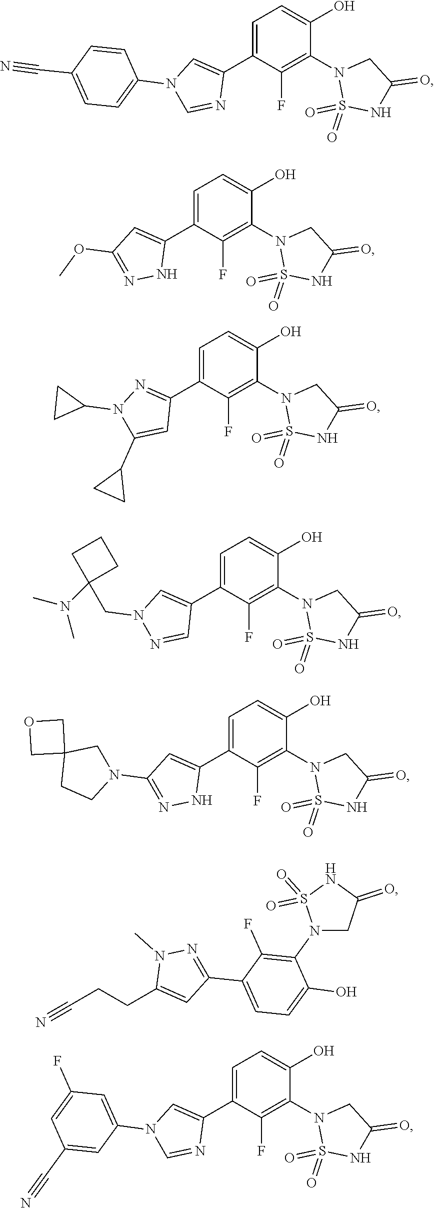 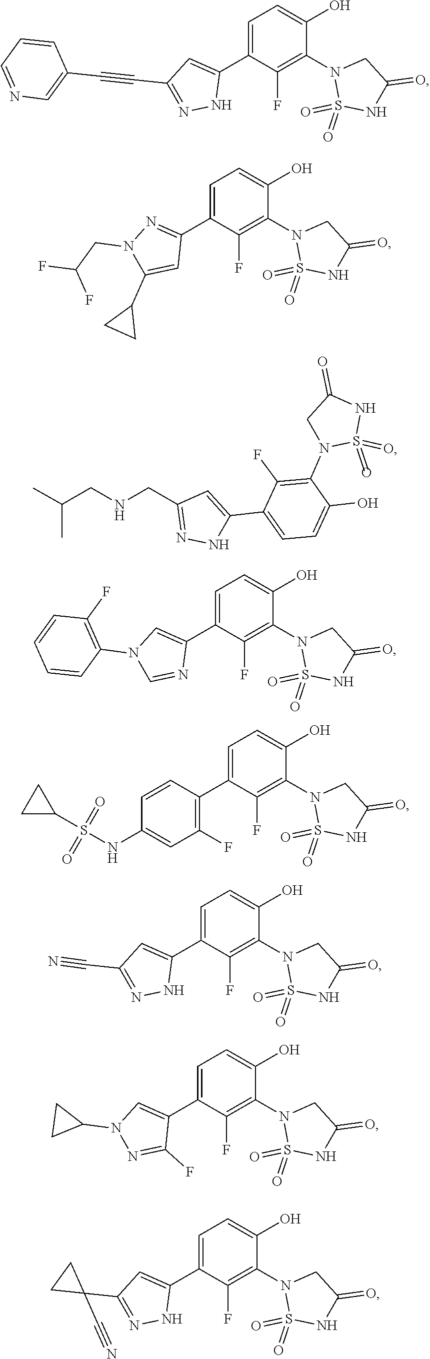 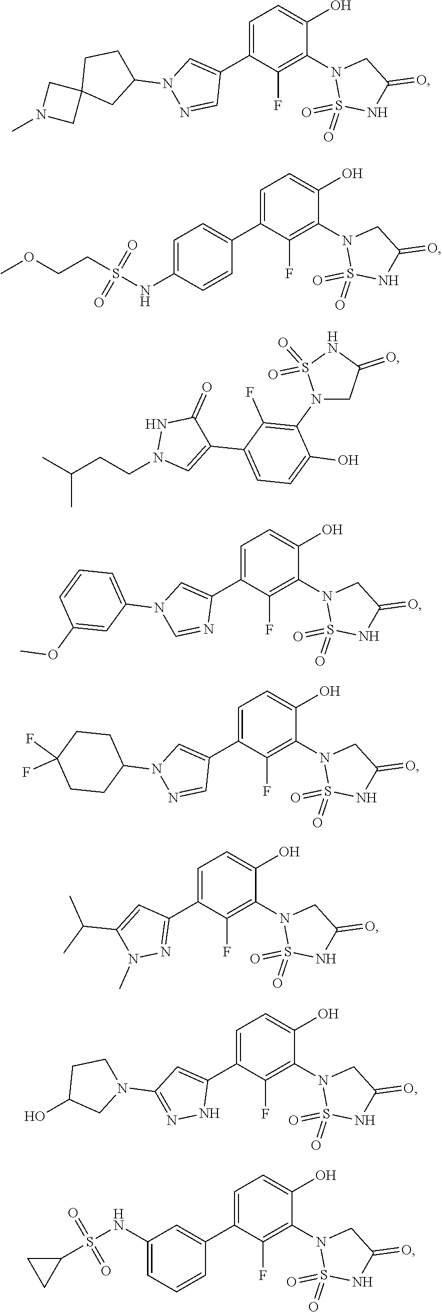 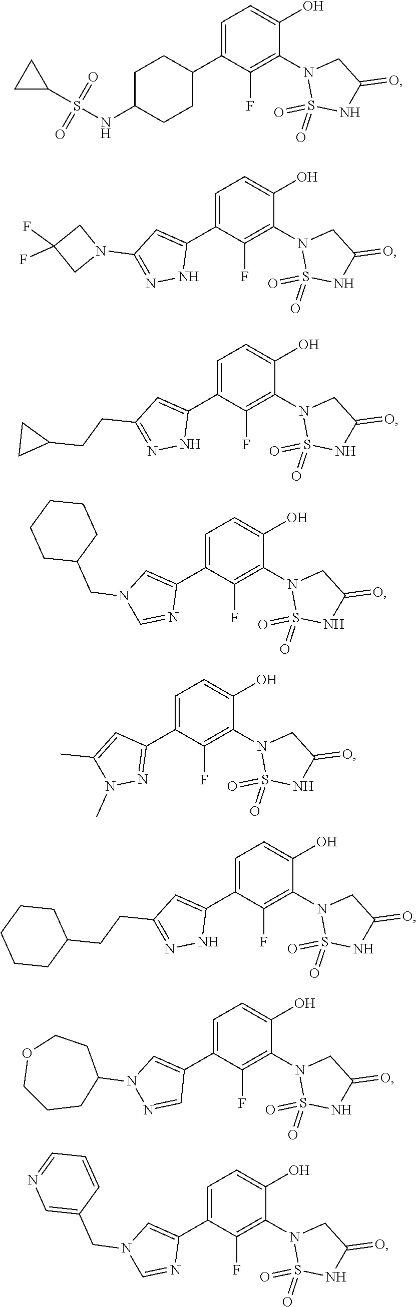 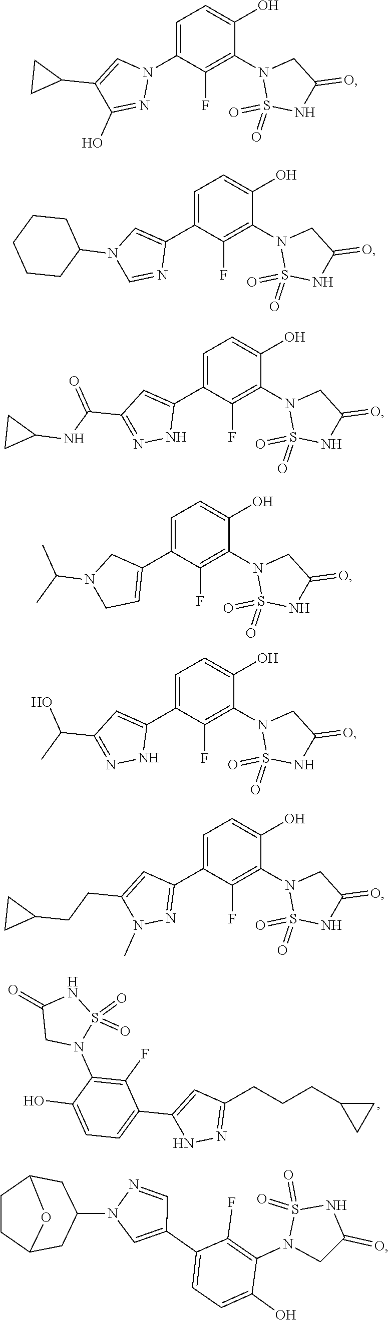 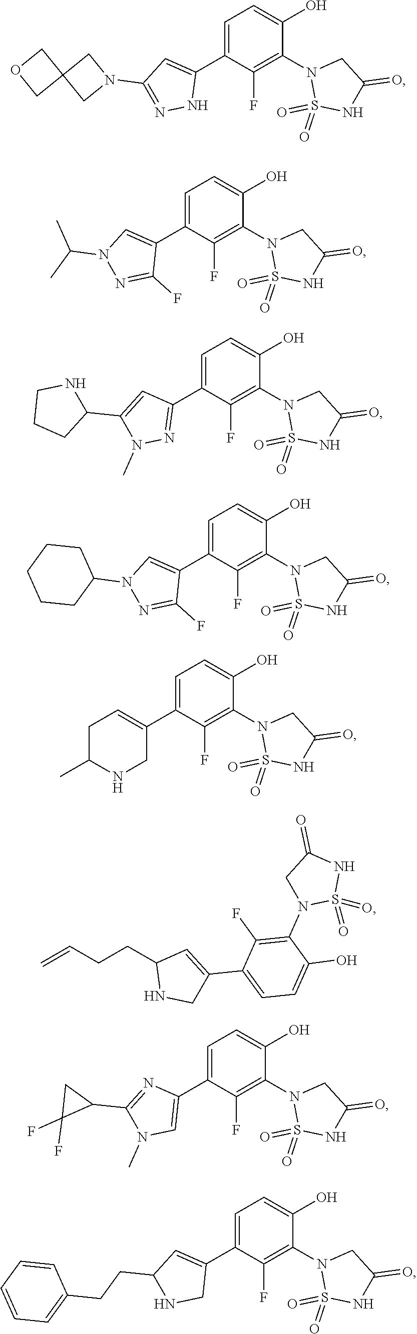 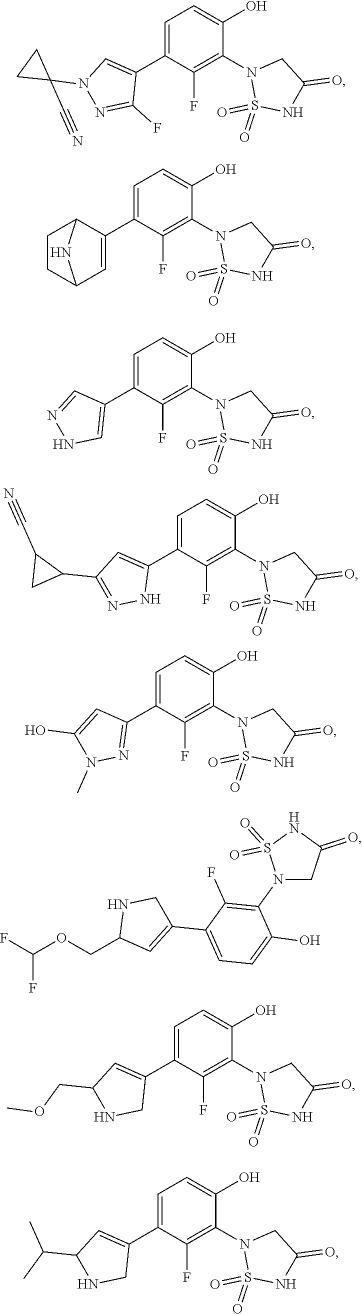 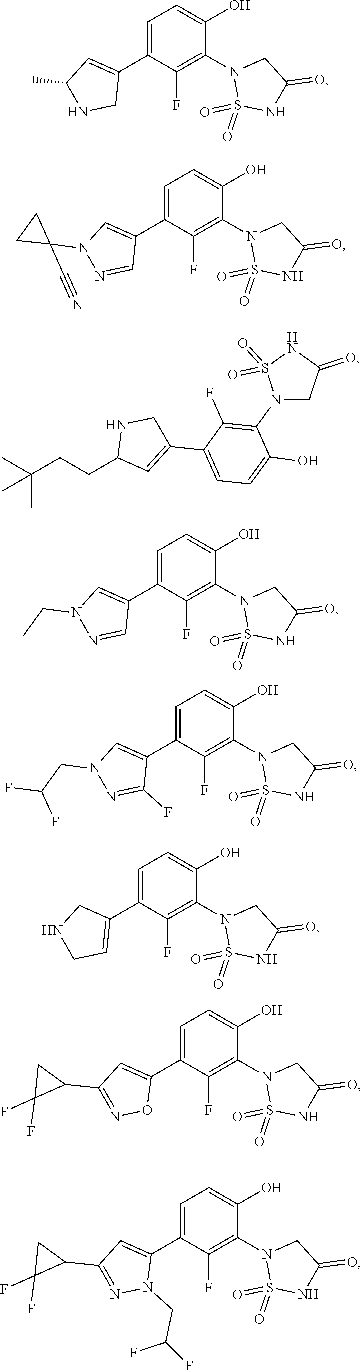 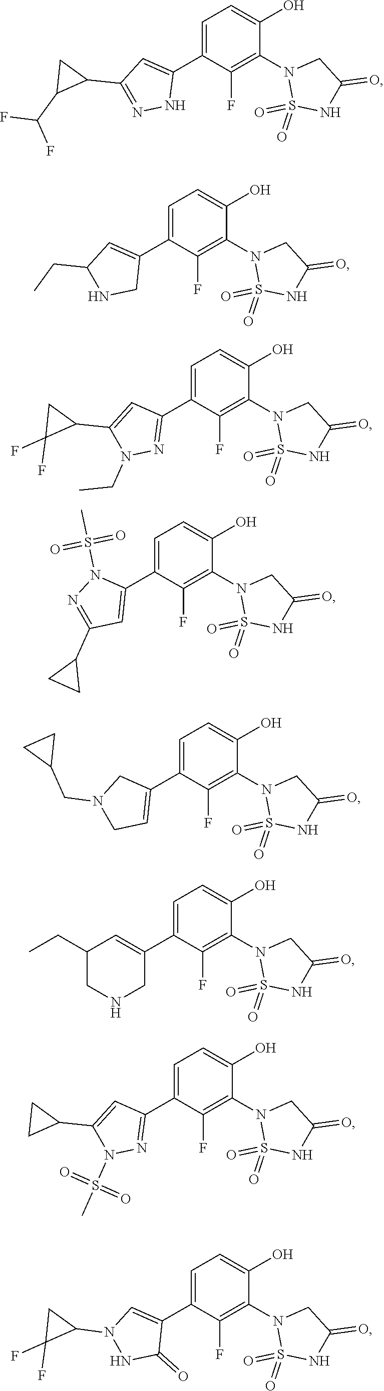 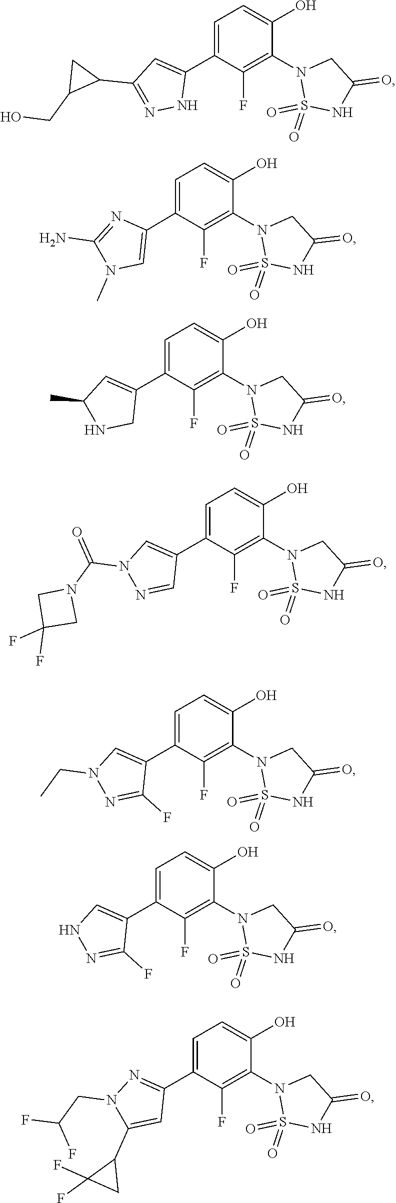 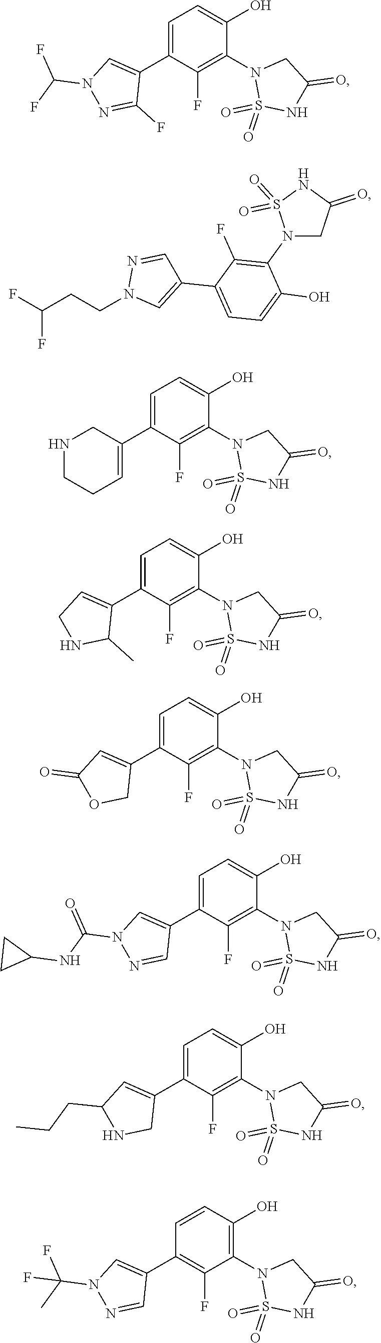 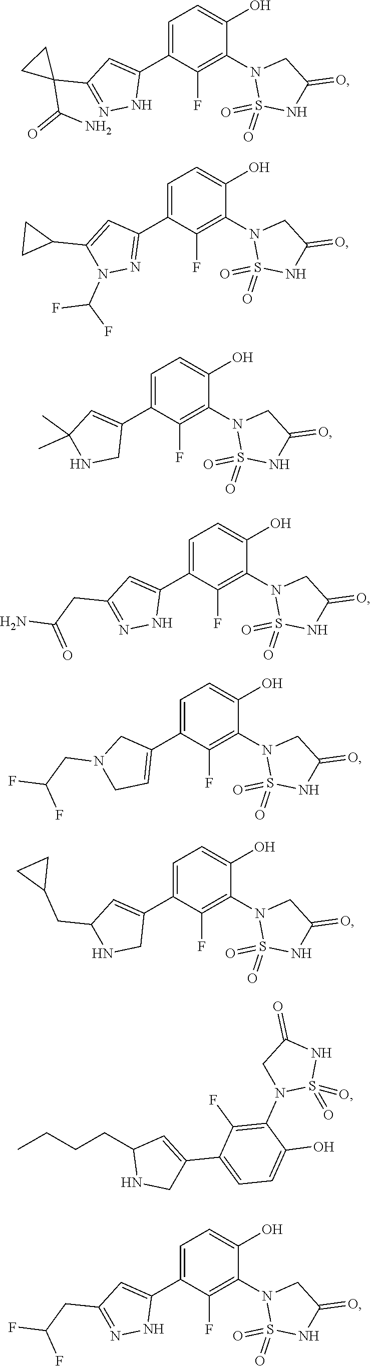 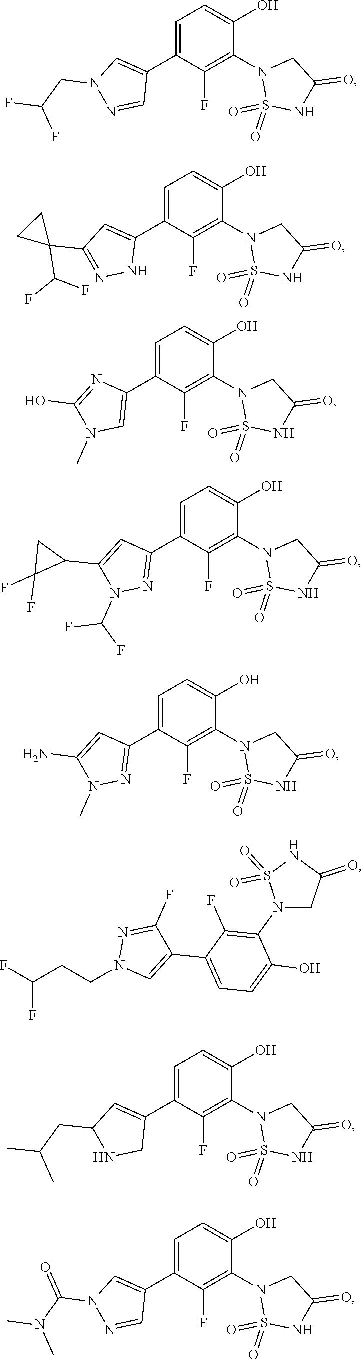 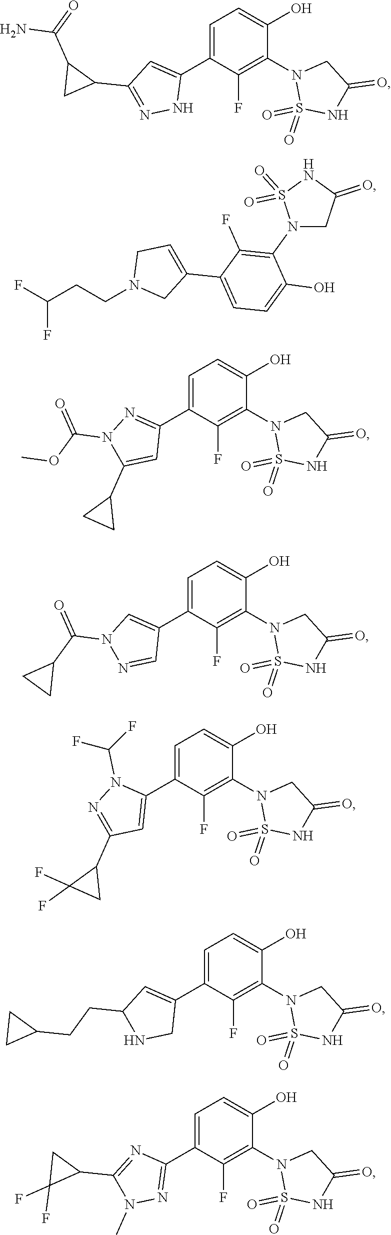 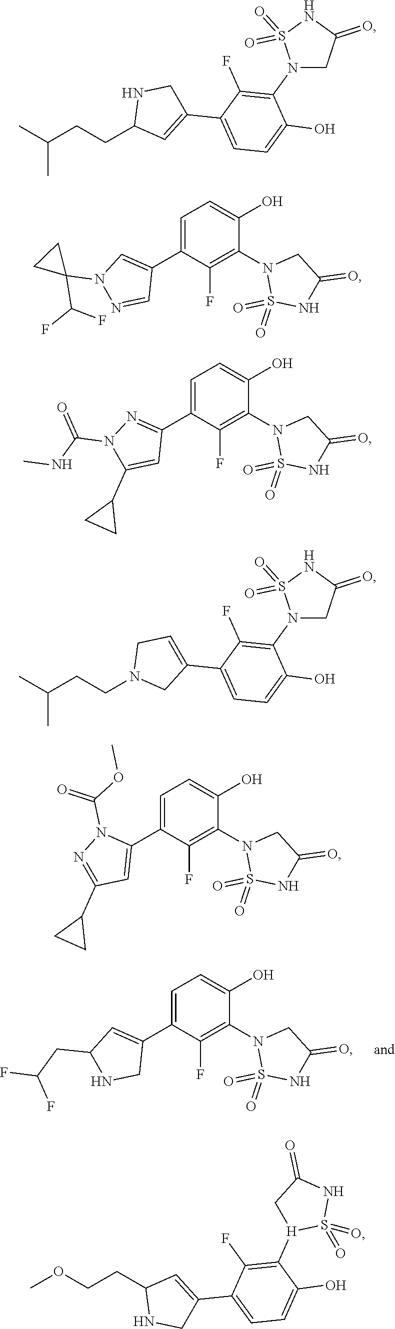
or a pharmaceutically acceptable salt or solvate thereof.

69. (canceled)

70. A pharmaceutical composition comprising a compound of claim 24, or a pharmaceutically acceptable salt or solvate thereof, and a pharmaceutically acceptable excipient.

71-90. (canceled)

91. A method of potentiating immunity of a subject in need thereof, comprising:
(a) selecting the subject that exhibits expression or activity of PTPN2 and/or PTP 1B; and
(b) downregulating expression or activity of PTPN2 and/or PTP1B by a compound of claim 24 in a cell of the subject, thereby potentiating immunity of the subject.

92-152. (canceled)

153. The method of claim 91, wherein the downregulating the expression or activity of PTPN2 and/or PTP 1B in the cell of the subject is performed prior to concurrent with or subsequent to an administration of another agent or therapy to the subject.

154-194. (canceled)
